(19) United States
(12) Patent Application Publication
(10) Pub. No.: US 2024/0301377 A1
(43) Pub. Date: Sep. 12, 2024

(54) Polynucleotides, Compositions, and Methods for Genome Editing

(71) Applicant: Intellia Therapeutics, Inc., Cambridge, MA (US)

(72) Inventors: Sabin Mulepati, Somerville, MA (US); Lindsey Jean Stretz, Cambridge, MA (US)

(73) Assignee: Intellia Therapeutics, Inc., Cambridge, MA (US)

(21) Appl. No.: 18/652,180

(22) Filed: May 1, 2024

Related U.S. Application Data
(63) Continuation of application No. PCT/US2022/079124, filed on Nov. 2, 2022.
(60) Provisional application No. 63/352,158, filed on Jun. 14, 2022.
(60) Provisional application No. 63/275,425, filed on Nov. 3, 2021.

(51) Int. Cl. C12N 9/22; A61K 38/00; A61K 48/00; C12N 9/78; C12N 15/11; C12N 15/63

(52) U.S. Cl. CPC C12N 9/22; A61K 48/0016; C12N 9/78; C12N 15/111; C12N 15/63; A61K 38/00; C07K 2319/09; C12N 2310/20; C12N 2830/50; C12Y 305/04005

(57) ABSTRACT

Compositions and methods for gene editing are provided. In some embodiments, provided is a polynucleotide encoding an RNA-guided DNA binding agent such as N. meningitidis Cas9 that can provide one or more of improved editing efficiency, reduced immunogenicity, or other benefits.

This application is a continuation of International Application No. PCT/US2022/079124 filed Nov. 2, 2022, which claims the benefit of priority to U.S. Provisional Application No. 63/275,425 filed on Nov. 3, 2021, and U.S. Provisional Application No. 63/352,158 filed on Jun. 14, 2022, the contents all of which are incorporated by reference in their entirety.

The instant application contains a Sequence Listing which has been submitted electronically in XML format and is hereby incorporated by reference in its entirety. Said XML copy, created on Nov. 1, 2022, is named 01155-0049-00PCT_ST26 and is 11,070,539 bytes in size.

The present disclosure relates to polynucleotides, compositions, and methods for genome editing involving RNA-guided DNA binding agents such as CRISPR-Cas systems and subunits thereof.

RNA-guided DNA binding agents such as CRISPR-Cas systems can be used for targeted genome editing, including in eukaryotic cells and in vivo. Such editing has been shown to be capable of inactivating certain deleterious alleles or correcting certain deleterious point mutations. For example, Neisseria meningitidis Cas9 (NmeCas9) has an advantageously low off-target cleavage rate. RNA-guided DNA binding agents can be produced in situ by cells contacted with polynucleotides, such as mRNAs or expression constructs. Existing approaches, e.g., in certain cell types or organisms such as mammals, may, however, provide less robust expression than desired or may be undesirably immunogenic, e.g., may provoke an undesirable elevation in cytokine levels.

Thus, there is a need for polynucleotides, compositions, and methods for expression of polypeptides, such as NmeCas9. The present disclosure aims to provide compositions and methods for polypeptide expression that provide one or more benefits such as at least one of improved expression levels, increased activity of the encoded polypeptide, or reduced immunogenicity (e.g., reduced elevation in cytokines upon administration), or at least to provide the public with a useful choice. In some embodiments, a polynucleotide encoding an RNA-guided DNA binding agent (e.g., NmeCas9) is provided, wherein one or more of its coding sequence, codon usage, non-coding sequence (e.g., a UTR), or heterologous domain (e.g., NLS) differs from existing polynucleotides in a manner disclosed herein. It has been found that such features can provide benefits such as those described above. In some embodiments, the improved editing efficiency occurs in or is specific to an organ or cell type of a mammal, such as the liver or hepatocytes.

The following embodiments are provided by this disclosure.

In some embodiments, a polynucleotide is provided, the polynucleotide comprising an open reading frame (ORF), the ORF comprising: a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, or 301-303, and 317-321, wherein the Nine Cas9 is an Nme2 Cas9, an Nme1 Cas9, or Nme3 Cas9; and a nucleotide sequence encoding a first nuclear localization signal (NLS).

In some embodiments, the ORF further comprises a nucleotide sequence encoding a second NLS. In some embodiments, the first and second NLS are independently selected from SEQ ID NO: 388 and 410-422. In some embodiments, the polynucleotide further comprises a poly-A sequence or a polyadenylation signal sequence.

In some embodiments, the ORF further comprises a nucleotide sequence encoding a linker sequence between the first NLS and the second NLS. In some embodiments, the ORF further comprises a nucleotide sequence encoding a linker spacer sequence between the Nine Cas9 coding sequence and the NLS proximal to the Nine Cas9 coding sequence. In some embodiments, the ORF Nine Cas9 has double stranded endonuclease activity. In some embodiments, the ORF Nine Cas9 has nickase activity. In some embodiments, the ORF the Nine Cas9 comprises a dCas9 DNA binding domain.

The following numbered embodiments provide additional support for and descriptions of the embodiments herein.

Embodiment 1 is a polynucleotide comprising an open reading frame (ORF), the ORF comprising: a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321, wherein the Nine Cas9 is an Nme2 Cas9, an Nme1 Cas9, or Nme3 Cas9; and a nucleotide sequence encoding a first nuclear localization signal (NLS).

Embodiment 2 is a polynucleotide of Embodiment 1, wherein the ORF further comprises a nucleotide sequence encoding a second NLS.

Embodiment 3 is a polynucleotide of Embodiment 1, wherein the first and second NLS are independently selected from SEQ ID NO: 388 and 410-422.

Embodiment 4 is a polynucleotide of any one of Embodiments 1-3, wherein the polynucleotide further comprises a poly-A sequence or a polyadenylation signal sequence.

Embodiment 5 is a polynucleotide of Embodiment 4, wherein the poly-A sequence comprises non-adenine nucleotides.

Embodiment 6 is a polynucleotide of any one of Embodiments 4-5, wherein the poly-A sequence comprises 100-400 nucleotides.

Embodiment 7 is a polynucleotide of any one of Embodiments 4-6, wherein the poly-A sequence comprises a sequence of SEQ ID NO: 409.

Embodiment 8 is a polynucleotide of any one of Embodiments 1-7, wherein the ORF further comprises a nucleotide sequence encoding a linker sequence between the first NLS and the second NLS.

Embodiment 9 is a polynucleotide of any one of Embodiments 1-8, wherein the ORF further comprises a nucleotide sequence encoding a linker spacer sequence between the Nine Cas9 coding sequence and the NLS proximal to the Nine Cas9 coding sequence.

Embodiment 10 is a polynucleotide of any one of Embodiments 8-9, wherein the linker comprises at least 1, at least 2, at least 3, at least 4, at least 5, at least 6, at least 7, at least 8, at least 9, or at least 10 amino acids.

Embodiment 11 is a polynucleotide of any one of Embodiments 8-10, wherein the linker sequence comprises GGG or GGGS, optionally wherein the GGG or GGGS sequence is at the N-terminus of the spacer sequence.

Embodiment 12 is a polynucleotide of any one of Embodiments 8-11, wherein the linker sequence comprises a sequence of any one of SEQ ID NOs: 61-122.

Embodiment 13 is a polynucleotide of any one of Embodiments 1-12, wherein the ORF further comprises one or more additional heterologous functional domains.

Embodiment 14 is a polynucleotide of any one of Embodiments 1-13, wherein the Nine Cas9 has double stranded endonuclease activity.

Embodiment 15 is a polynucleotide of any one of Embodiments 1-14, wherein the Nine Cas9 has nickase activity.

Embodiment 16 is a polynucleotide of any one of Embodiments 1-14, wherein the Nine Cas9 comprises a dCas9 DNA binding domain.

Embodiment 17 is a polynucleotide of any one of Embodiments 1-16, wherein the NmeCas9 comprises an amino acid sequence with at least 91%, 92%, 93%, 94%, 95%, 96%, 97%, 98%, or 99% to any one of SEQ ID NOs: 1 and 4-13, 220, 227, 234, 241, 248, 255, 262, 269, 276, 283, 290, 297, or 310-315.

Embodiment 18 is a polynucleotide of any one of Embodiments 1-17 wherein the NmeCas9 comprises an amino acid sequence of SEQ ID NO: 1 and 4-13, 220, 227, 234, 241, 248, 255, 262, 269, 276, 283, 290, 297, or 310-315.

Embodiment 19 is a polynucleotide of any one of Embodiments 1-18, wherein the sequence encoding the NmeCas9 comprises a nucleotide sequence having at least 91%, 92%, 93%, 94%, 95%, 96%, 97%, 98%, or 99% identity to the nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

Embodiment 20 is a polynucleotide of any one of Embodiments 1-19, wherein the sequence encoding the NmeCas9 comprises a nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

Embodiment 21 is a polynucleotide comprising an open reading frame (ORF) encoding a polypeptide comprising: a cytidine deaminase, which is optionally an APOBEC3A deaminase; a nucleotide sequence encoding a C-terminal N. meningitidis (Nine) Cas9 nickase polypeptide at least 90% identical to any one of SEQ ID NOs: 1 and 4-13, 220, 227, 234, 241, 248, 255, 262, 269, 276, 283, 290, or 297, wherein the Nine Cas9 nickase is an Nme2 Cas9 nickase, an Nme1 Cas9 nickase, or an Nme3 Cas9 nickase; and a nucleotide sequence encoding a first nuclear localization signal (NLS); wherein the polypeptide does not comprise a uracil glycosylase inhibitor (UGI).

Embodiment 22 is a polynucleotide of Embodiment 21, wherein the ORF further comprises a nucleotide sequence encoding a second NLS.

Embodiment 23 is a polynucleotide of any one of Embodiments 21-22, wherein the deaminase is located N-terminal to an NLS in the polypeptide.

Embodiment 24 is a polynucleotide of any one of Embodiments 21-23, wherein the cytidine deaminase is located N-terminal to the first NLS and the second NLS in the polypeptide.

Embodiment 25 is a polynucleotide of any one of Embodiments 21-22, wherein the cytidine deaminase is located C-terminal to an NLS in the polypeptide.

Embodiment 26 is a polynucleotide of any one of Embodiments 23-25, wherein the cytidine deaminase is located C-terminal to the first NLS and the second NLS in the polypeptide.

Embodiment 27 is a polynucleotide of any one of Embodiments 21-26, wherein the ORF does not comprise a coding sequence for an NLS C-terminal to the ORF encoding the Nine Cas9.

Embodiment 28 is a polynucleotide of any one of Embodiments 21-26, wherein the ORF does not comprise a coding sequence C-terminal to the ORF encoding the Nine Cas9.

Embodiment 29 is a polynucleotide of any one of Embodiments 1-28, wherein the cytidine deaminase comprises an amino acid sequence with at least 87% identity to SEQ ID NOs: 151.

Embodiment 30 is a polynucleotide of any one of Embodiments 1-28, wherein the cytidine deaminase comprises an amino acid sequence with at least 80% identity to SEQ ID NOs: 152-216.

Embodiment 31 is a polynucleotide of any one of Embodiments 1-28, wherein the cytidine deaminase comprises an amino acid sequence with at least 80% identity to SEQ ID NO: 14.

Embodiment 32 is a polynucleotide of any one of Embodiments 1-31, the ORF comprises a nucleotide sequence at least 80% identical to SEQ ID NO: 42.

Embodiment 33 is a polynucleotide of any one of Embodiments 1-32, wherein the polynucleotide comprises a 5′ UTR with at least 90% identity to any one of SEQ ID NOs: 391-398.

Embodiment 34 is a polynucleotide of any one of Embodiments 1-33, wherein the polynucleotide comprises a 5′ UTR comprising any one of SEQ ID NOs: 391-398.

Embodiment 35 is a polynucleotide of any one of Embodiments 1-34, wherein the polynucleotide comprises a 3′ UTR with at least 90% identity to any one of SEQ ID NOs: 399-406.

Embodiment 36 is a polynucleotide of any one of Embodiments 1-35, wherein the polynucleotide comprises a 3′ UTR comprising any one of SEQ ID NOs: 399-306.

Embodiment 37 is a polynucleotide of any one of Embodiments 1-36, wherein the polynucleotide comprises a 5′ UTR and a 3′ UTR from the same source.

Embodiment 38 is a polynucleotide of any one of Embodiments 1-37, wherein the polynucleotide comprises a 5′ cap, optionally wherein the 5′ cap is Cap0, Cap1, or Cap2.

Embodiment 39 is a polynucleotide of any one of Embodiments 1-38, wherein at least 75%, 80%, 85%, 90%, 95%, 98%, 99%, or 100% of the codons of the ORF are minimal adenine codons or minimal uridine codons.

Embodiment 40 is a polynucleotide of any one of Embodiments 1-39, wherein the ORF comprises or consists of codons that increase translation of the mRNA in a mammal.

Embodiment 41 is a polynucleotide of any one of Embodiments 1-40, wherein the ORF comprises or consists of codons that increase translation of the mRNA in a human.

Embodiment 42 is a polynucleotide of any one of Embodiments 1-41, wherein the polynucleotide is an mRNA.

Embodiment 43 is a polynucleotide of Embodiment 42, wherein the ORF comprises a sequence having at least 90%, 95%, 98% or 100% identity to any one of SEQ ID NO: 29, 32-41, 224-226, 231-233, 238-240, 245-247, 252-254, 259-261, 266-268, 273-275, 280-282, 287-289, 294-296, 301-303, or 316-321.

Embodiment 44 is a polynucleotide of any one of Embodiments 42-43, wherein at least 10% of the uridine in the mRNA is substituted with a modified uridine.

Embodiment 45 is a polynucleotide of any one of Embodiments 42-43, wherein less than 10% of the uridine in the mRNA is substituted with a modified uridine.

Embodiment 46 is a polynucleotide of Embodiment 45, wherein the modified uridine is one or more of N1-methyl-pseudouridine, pseudouridine, 5-methoxyuridine, or 5-iodouridine.

Embodiment 47 is a polynucleotide of Embodiment 45, wherein the modified uridine is one or both of N1-methyl-pseudouridine or 5-methoxyuridine.

Embodiment 48 is a polynucleotide of any one of Embodiments 45-47, wherein the modified uridine is N1-methyl-pseudouridine.

Embodiment 49 is a polynucleotide of any one of Embodiments 45-47, wherein the modified uridine is 5-methoxyuridine.

Embodiment 50 is a polynucleotide of any one of Embodiments 44, and 46-49, wherein 15% to 45% of the uridine is substituted with the modified uridine.

Embodiment 51 is a polynucleotide of Embodiment 50, wherein at least 20% or at least 30% of the uridine is substituted with the modified uridine.

Embodiment 52 is a polynucleotide of Embodiment 51, wherein at least 80% or at least 90% of the uridine is substituted with the modified uridine.

Embodiment 53 is a polynucleotide of Embodiment 52, wherein 100% of the uridine is substituted with the modified uridine.

Embodiment 54 is a polynucleotide of Embodiment 42, wherein less than 10% of the nucleotides in the mRNA is substituted with a modified nucleotide.

Embodiment 55 is a composition comprising the polynucleotide according to any one of Embodiments 1-54, and at least one guide RNA (gRNA).

Embodiment 56 is a composition comprising a first polynucleotide comprising a first open reading frame (ORF) encoding a polypeptide comprising a cytidine deaminase, optionally an APOBEC3A deaminase, and a NmeCas9 nickase, and a second polynucleotide comprising a second open reading frame encoding a uracil glycosylase inhibitor (UGI), wherein the second polynucleotide is different from the first polynucleotide, and optionally further comprising a guide RNA (gRNA).

Embodiment 57 is a composition of Embodiment 55 or 56, wherein the gRNA is a single guide RNA.

Embodiment 58 is a composition of Embodiment 55 or 56, wherein the gRNA is a dual guide RNA.

Embodiment 59 is a composition comprising the polynucleotide according to any one of Embodiments 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

Embodiment 60 is a composition comprising the polynucleotide according to any one of Embodiments 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.

Embodiment 61 is a polypeptide encoded by the polynucleotide of any one of Embodiments 1-60.

Embodiment 62 is a vector comprising the polynucleotide of any one of Embodiments 1-60.

Embodiment 63 is an expression construct comprising a promoter operably linked to a sequence encoding the polynucleotide of any one of Embodiments 1-60.

Embodiment 64 is an expression construct of Embodiment 63, wherein the promoter is an RNA polymerase promoter, optionally a bacterial RNA polymerase promoter.

Embodiment 65 is an expression construct of Embodiment 63 or 64, further comprising poly-A tail sequence or a polyadenylation signal sequence.

Embodiment 66 is an expression construct of Embodiment 65, wherein the poly-A tail sequence is an encoded poly-A tail sequence.

Embodiment 67 is a plasmid comprising the expression construct of any one of Embodiments 63-66.

Embodiment 68 is a host cell comprising the vector of Embodiment 62, the expression construct of any one of Embodiments 63-66, or the plasmid of Embodiment 67.

Embodiment 69 is a pharmaceutical composition comprising the polynucleotide, composition, or polypeptide of any of Embodiments 1-61 and a pharmaceutically acceptable carrier.

Embodiment 70 is a kit comprising the polynucleotide, composition, or polypeptide of any of Embodiments 1-61.

Embodiment 71 is use of the polynucleotide, composition, or polypeptide of any one of Embodiments 1-61 for modifying a target gene in a cell.

Embodiment 72 is use of the polynucleotide, composition, or polypeptide of any one of Embodiments 1-61 for the manufacture of a medicament for modifying a target gene in a cell.

Embodiment 73 is a polynucleotide or composition of any one of Embodiments 1-60, wherein the polynucleotide or composition is formulated as a lipid nucleic acid assembly composition, optionally a lipid nanoparticle.

Embodiment 74 is a method of modifying a target gene comprising delivering to a cell the polynucleotide, polypeptide, or composition of any one of Embodiments 1-61.

Embodiment 75 is a method of modifying a target gene, comprising delivering to the cell one or more lipid nucleic acid assembly compositions, optionally lipid nanoparticles, comprising the polynucleotide according to any one of Embodiments 1-60, and one or more guide RNAs.

Embodiment 76 is a method of any one of Embodiments 74-75, wherein at least one lipid nucleic acid assembly composition comprises lipid nanoparticle (LNPs), optionally wherein all lipid nucleic acid assembly compositions comprise LNPs.

Embodiment 77 is a method of any one of Embodiments 74-75, wherein at least one lipid nucleic acid assembly composition is a lipoplex composition.

Embodiment 78 is a composition or method of any one of Embodiments 75-77, wherein the lipid nucleic acid assembly composition comprises an ionizable lipid.

Embodiment 79 is a method of producing a polynucleotide of any one of Embodiments 1-54, comprising contacting the expression construct of Embodiments 63-66 with an RNA polymerase and NTPs that comprise at least one modified nucleotide.

Embodiment 80 is a method of Embodiment 79, wherein NTPs comprise one modified nucleotide.

Embodiment 81 is a method of Embodiment 79 or 80 wherein the modified nucleotide comprises a modified uridine.

Embodiment 82 is a method of Embodiment 81, wherein at least 80% or at least 90% or 100% of the uridine positions are modified uridines.

Embodiment 83 is a method of Embodiment 81 or 82, wherein the modified uridine comprises or is a substituted uridine, pseudouridine, or a substituted pseudouridine, optionally N1-methyl-psuedouridine.

Embodiment 84 is a method of any one of Embodiments 79-83, wherein the expression construct comprises an encoded poly-A tail sequence.

### BRIEF DESCRIPTION OF THE DRAWINGS

FIG. 1 shows mean percent editing at the TTR locus in PMH with increasing doses of Nme2Cas9 mRNA and chemically modified sgRNA.

FIG. 2A shows mean percent editing at the TTR locus in PMH using varying ratios of sgRNA and Nme2Cas9 mRNA.

FIG. 2B shows mean percent editing at the TTR locus in PMH using varying ratios pgRNA and Nme2Cas9 mRNA.

FIG. 3 shows mean percent editing at the TTR locus in PMH for pgRNAs with Nme2Cas9 mRNA.

FIG. 4 shows mean editing percentage in at the PCSK9 locus in PMH.

FIG. 5A shows mean editing results at the VEGFA locus in HEK cells treated with mRNA C

FIG. 5B shows mean editing results at the VEGFA locus in HEK cells treated with mRNA I

FIG. 5C shows mean editing results at the VEGFA locus in HEK cells treated with mRNA J

FIG. 5D shows mean editing results at the VEGFA locus in PHH cells treated with mRNA C

FIG. 5E shows mean editing results at the VEGFA locus in PHH cells treated with mRNA I

FIG. 5F shows mean editing results at the VEGFA locus in PHH cells treated with mRNA J

FIG. 6 shows mean percent editing at the mouse TTR locus in PMH cells treated with NmeCas9 constructs designed with 1 or 2 nuclear localization sequences.

FIG. 7 shows mean percent editing at the mouse TTR locus in PMH cells treated with pgRNA and various Nme2Cas9 mRNAs.

FIG. 8 shows fold change in Nme2Cas9 protein expression compared to SpyCas9 protein expression in PMH, PRH, PCH and PHH cells.

FIGS. 9A-9F show fold change in Nme2Cas9 protein expression compared to SpyCas9 protein expression in T cells from 2 donors assayed at 24 hours, 48 hours and 72 hours after treatment.

FIG. 10 shows mean percent editing at the TTR locus in mouse liver treated with sgRNA and Nme2Cas9.

FIG. 11A shows mean percent editing at the TTR locus in mouse liver following treatment with pgRNA and Nme2Cas9.

FIG. 11B shows mean serum TTR protein following treatment with pgRNA and Nme2Cas9.

FIG. 11C shows mean percent TTR knockdown following treatment with pgRNA and Nme2Cas9.

FIG. 11D shows mean percent editing at the TTR locus in mouse liver following treatment with pgRNA and various Nme2Cas9.

FIG. 11E shows serum TTR protein knockdown following treatment with pgRNA and various Nme2Cas9.

FIG. 12 shows mean percent editing in mouse liver following treatment with various Nme2Cas9 constructs.

FIG. 13 shows mean percent editing in mouse liver following treatment with pgRNA and various Nme2Cas9

FIG. 14 shows mean percent editing in mouse liver following treatment with various base editors.

FIG. 15 shows an exemplary schematic of Nme2 sgRNA in a possible secondary structure, including the repeat/anti-repeat region and the hairpin region which includes hairpin 1 and hairpin 2 regions and further indicates the guide region (or targeting region) (denoted with a gray fill with dashed outline), bases not amenable to single or pairwise deletion (denoted with a gray fill with solid outline), bases amenable to single or pairwise deletion (open circles).

FIG. 16 shows the mean percent CD3 negative T cells following TRAC editing with Nme1Cas9.

FIG. 17 shows the mean percent CD3 negative T cells following TRAC editing with Nme3Cas9.

FIG. 18 shows the expression of Nme-HiBiT constructs in T cells at 24 hours.

FIG. 19 shows the CD3-negative cell population as a function of NmeCas9 mRNA amount.

FIG. 20 shows the dose response curve for select gRNAs in PCH.

FIG. 21 shows the dose response curve for LNP dilution series in PCH.

FIG. 22 shows serum TTR levels in mice.

FIG. 23 shows percent editing at the TTR locus in mouse liver samples.

FIG. 24 shows the dose response curve for select gRNAs in PMH.

FIG. 25 shows the dose response curve for select gRNAs in PMH.

FIG. 26 shows mean percent editing at PCSK9 locus in PMH with modified sgRNAs.

FIG. 27 shows mean percent editing in PMH of several Nme2Cas9 mRNAs with a modified sgRNA.

FIG. 28 shows the percent editing at the TTR locus in primary mouse hepatocytes.

FIG. 29 shows serum TTR levels in mice.

FIG. 30 shows percent editing at the TTR locus in mouse liver samples.

FIG. 31 shows serum TTR measurements following treatment in mice.

FIG. 32 shows percent editing at the TTR locus in mouse liver samples.

FIG. 33 shows an exemplary sgRNA (G021536; SEQ ID NO: 139) in a possible secondary structure. The methylation is shown in bold; phosphorothioate linkages are indicated by ‘*’. Watson-Crick base pairing is indicated by a ‘−’ between nucleotides in duplex portions. Non-Watson-Crick base pairing is indicated by a ‘•’ between nucleotides in duplex portions.

FIG. 34 shows an exemplary sgRNA (G032572; SEQ ID NO: 528) in a possible secondary structure. The unmodified nucleotides are shown in bold and methylation is shown in light fonts; phosphorothioate linkages are indicated by ‘*’. Watson-Crick base pairing is indicated by a ‘−’ between nucleotides in duplex portions. Non-Watson-Crick base pairing is indicated by a ‘•’ between nucleotides in duplex portions.

FIG. 35 shows an exemplary sgRNA (G031771; SEQ ID NO: 529) in a possible secondary structure. The unmodified nucleotides are shown in bold and methylation is shown in light fonts; phosphorothioate linkages are indicated by ‘*’. Watson-Crick base pairing is indicated by a ‘−’ between nucleotides in duplex portions. Non-Watson-Crick base pairing is indicated by a ‘•’ between nucleotides in duplex portions.

BRIEF DESCRIPTION OF DISCLOSED SEQUENCES

| SEQ ID NO | Description |
|---|---|
| 1 | Exemplary Amino acid sequence for Nme2Cas9 |
| 2 | Exemplary Amino acid sequence for SpyCas9 base editor |
| 3 | Exemplary Amino acid sequence for UGI |
| 4-8 | Exemplary Amino acid sequence for Nme2Cas9 |
| 9 | Exemplary Amino acid sequence for Nme2Cas9 with HiBiT tag |
| 10 | Exemplary Amino acid sequence for Nme2Cas9 |
| 11 | Exemplary Amino acid sequence for Nme2Cas9 |
| 12 | Exemplary Amino acid sequence for Nme2Cas9 with HiBiT tag |
| 13 | Exemplary Amino acid sequence for Nme2Cas9 |
| 14 | Exemplary Amino acid sequence for Nme2Cas9 base editor |
| 15 | Exemplary mRNA encoding Nme2Cas9 |
| 16 | Exemplary mRNA encoding SpyCas9 base editor |
| 17 | Exemplary mRNA encoding UGI |
| 18-22 | Exemplary mRNA encoding Nme2Cas9 |
| 23 | Exemplary encoding Nme2Cas9 with HiBiT tag |
| 24 | Exemplary mRNA encoding Nme2Cas9 |
| 25 | Exemplary mRNA encoding Nme2Cas9 |
| 26 | Exemplary encoding Nme2Cas9 with HiBiT tag |
| 27 | Exemplary mRNA encoding Nme2Cas9 |
| 28 | Exemplary mRNA encoding Nme2Cas9 base editor |
| 29 | Open reading frame for Nme2Cas9 |
| 30 | Open reading frame for SpyCas9 base editor |
| 31 | Open reading frame for UGI |
| 32-36 | Open reading frame for Nme2Cas9 |
| 37 | Open reading frame for Nme2Cas9 with HiBiT tag |
| 38 | Open reading frame sequences for Nme2Cas9 |
| 39 | Open reading frame sequences for Nme2Cas9 |
| 40 | Open reading frame for Nme2Cas9 with HiBiT tag |
| 41 | Open reading frame sequences for Nme2Cas9 |
| 42 | Open reading frame for Nme2Cas9 base editor |
| 43-47 | ORF encoding Sp. Cas9 |
| 48 | amino acid sequence for Sp. Cas9 |
| 49 | Open reading frame for Cas9 with HiBiT tag |
| 50 | Amino acid sequence for Cas9 with HiBiT tag |
| 51-57 | Not Used |
| 58-122 | Exemplary amino acid sequences for peptide linker |
| 123-129 | Not Used |
| 130-150 | Exemplary guide RNA sequences |
| 151-216 | Exemplary amino acid sequences for cytidine deaminases |
| 217-219 | Not Used |
| 220 | Exemplary amino acid sequence of Nme1Cas9 cleavase |
| 221-223 | Exemplary coding sequences encoding Nme1Cas9 cleavase |
| 224-226 | Exemplary open reading frame for Nme1Cas9 cleavase |
| 227 | Exemplary amino acid sequence of Nme1Cas9 dCas9 |
| 228-230 | Exemplary coding sequence encoding Nme1Cas9 dCas9 |
| 231-233 | Exemplary open reading frame for Nme1Cas9 dCas9 |
| 234 | Exemplary amino acid sequence of Nme1Cas9 RuvC nickase |
| 235-237 | Exemplary coding sequence encoding Nme1Cas9 RuvC nickase |
| 238-240 | Exemplary open reading frame for Nme1Cas9 RuvC nickase |
| 241 | Exemplary amino acid sequence of Nme1Cas9 HNH nickase |
| 242-244 | Exemplary coding sequence encoding Nme1Cas9 HNH nickase |
| 245-247 | Exemplary open reading frame for Nme1Cas9 HNH nickase |
| 248 | Exemplary amino acid sequence of Nme2Cas9 cleavase |
| 249-251 | Exemplary coding sequence encoding Nme2Cas9 cleavase |
| 252-254 | Exemplary open reading frame for Nme2Cas9 cleavase |
| 255 | Exemplary amino acid sequence of Nme2Cas9 dCas9 |
| 256-258 | Exemplary coding sequence encoding Nme2Cas9 dCas9 |
| 259-261 | Exemplary open reading frame for Nme2Cas9 dCas9 |
| 262 | Exemplary amino acid sequence of Nme2Cas9 RuvC nickase |
| 263-265 | Exemplary coding sequence encoding Nme2Cas9 RuvC nickase |
| 266-268 | Exemplary open reading frame for Nme2Cas9 RuvC nickase |
| 269 | Exemplary amino acid sequence of Nme2Cas9 HNH nickase |
| 270-272 | Exemplary coding sequence encoding Nme2Cas9 HNH nickase |
| 273-275 | Exemplary open reading frame for Nme2Cas9 HNH nickase |
| 276 | Exemplary amino acid sequence of Nme3Cas9 cleavase |
| 277-279 | Exemplary coding sequence encoding Nme3Cas9 cleavase |
| 280-282 | Exemplary open reading frame for Nme3Cas9 cleavase |
| 283 | Exemplary amino acid sequence of Nme3Cas9 dCas9 |
| 284-286 | Exemplary coding sequence encoding Nme3Cas9 dCas9 |
| 287-289 | Exemplary open reading frame for Nme3Cas9 dCas9 |
| 290 | Exemplary amino acid sequence of Nme3Cas9 RuvC nickase |
| 291-293 | Exemplary coding sequence encoding Nme3Cas9 RuvC nickase |
| 294-296 | Exemplary open reading frame for Nme3Cas9 RuvC nickase |
| 297 | Exemplary amino acid sequence of Nme3Cas9 HNH nickase |
| 298-300 | Exemplary coding sequence encoding Nme3Cas9 HNH nickase |
| 301-303 | Exemplary open reading frame for Nme3Cas9 HNH nickase |
| 304 | Exemplary coding sequence encoding Nme2Cas9 (mRNA AA) |
| 305 | Exemplary coding sequence encoding Nme1Cas9 (mRNA AB) |
| 306 | Exemplary coding sequence encoding Nme2Cas9 with HiBiT tag (mRNA V) |
| 307 | Exemplary coding sequence encoding Nme1Cas9 with HiBiT tag (mRNA X) |
| 308 | Exemplary coding sequence encoding Nme1Cas9 with HiBiT tag (mRNA Y) |
| 309 | Exemplary coding sequence encoding Nme3Cas9 with HiBiT tag (mRNA Z) |
| 310 | Exemplary amino acid sequence for Nme2Cas9 (mRNA AA amino acid) |
| 311 | Exemplary amino acid sequence for Nme1Cas9 (mRNA AB amino acid) |
| 312 | Exemplary amino acid sequence for Nme2Cas9 with HiBiT tag (mRNA V amino |
| | acid) |
| 313 | Exemplary amino acid sequence for Nme1Cas9 with HiBiT tag (mRNA X amino |
| | acid) |
| 314 | Exemplary amino acid sequence for Nme1Cas9 with HiBiT tag (mRNA Y amino |
| | acid) |
| 315 | Exemplary amino acid sequence for Nme3Cas9 with HiBiT tag (mRNA Z amino |
| | acid) |
| 316 | Exemplary open reading frame for Nme2Cas9 (mRNA AA ORF) |
| 317 | Exemplary open reading frame for Nme1Cas9 (mRNA AB ORF) |
| 318 | Exemplary open reading frame for Nme2Cas9 with HiBiT tag (mRNA V ORF) |
| 319 | Exemplary open reading frame for Nme1Cas9 with HiBiT tag (mRNA X ORF) |
| 320 | Exemplary open reading frame for Nme1Cas9 with HiBiT tag (mRNA Y ORF) |
| 321 | Exemplary open reading frame for Nme3Cas9 with HiBiT tag (mRNA Z ORF) |
| 322-350 | Not used |
| 351-366 | Exemplary guide RNA sequences |
| 367-382 | Not used |
| 383 | SV40 NLS |
| 384 | SV40 NLS |
| 385 | bipartite NLS |
| 386 | c-myc like NLS |
| 387 | Nucleic acid sequence for SV40 NLS |
| 388 | Amino acid sequence for SV40 NLS |
| 389 | U6 promoter |
| 390 | CMV promoter |
| 391 | Exemplary 5′ UTR |
| 392 | Exemplary 5′ UTR |
| 393 | Exemplary 5′ UTR |
| 394 | Exemplary 5′ UTR |
| 395 | Exemplary 5′ UTR |
| 396 | Exemplary 5′ UTR |
| 397 | Exemplary 5′ UTR |
| 398 | Exemplary 5′ UTR |
| 399 | Exemplary 3′ UTR |
| 400 | Exemplary 3′ UTR |
| 401 | Exemplary 3′ UTR |
| 402 | Exemplary 3′ UTR |
| 403 | Exemplary 3′ UTR |
| 404 | Exemplary 3′ UTR |
| 405 | Exemplary 3′ UTR |
| 406 | Exemplary 3′ UTR |
| 407 | Exemplary Kozak sequence |
| 408 | Exemplary Kozak sequence |
| 409 | Exemplary poly-A sequence |
| 410 | Exemplary NLS 1 |
| 411 | Exemplary NLS 2 |
| 412 | Exemplary NLS 3 |
| 413 | Exemplary NLS 4 |
| 414 | Exemplary NLS 5 |
| 415 | Exemplary NLS 6 |
| 416 | Exemplary NLS 7 |
| 417 | Exemplary NLS 8 |
| 418 | Exemplary NLS 9 |
| 419 | Exemplary NLS 10 |
| 420 | Exemplary NLS 11 |
| 421 | Alternative SV40 NLS |
| 422 | Nucleoplasmin NLS |
| 423 | Exemplary coding sequence for SV40 NLS |
| 424 | Exemplary coding sequence for NLS1 |
| 425 | Exemplary coding sequence for NLS2 |
| 426 | Exemplary coding sequence for NLS3 |
| 427 | Exemplary coding sequence for NLS4 |
| 428 | Exemplary coding sequence for NLS5 |
| 429 | Exemplary coding sequence for NLS6 |
| 430 | Exemplary coding sequence for NLS7 |
| 431 | Exemplary coding sequence for NLS8 |
| 432 | Exemplary coding sequence for NLS9 |
| 433 | Exemplary coding sequence for NLS10 |
| 434 | Exemplary coding sequence for NLS11 |
| 435 | Exemplary coding sequence for alternate SV40 NLS |
| 436-458 | Exemplary NmeCas9 guide RNA sequences |
| 459-499 | Not Used |
| 500 | Wild-type NmeCas9 guide RNA |
| 501 | Shortened/unmodified NmeCas9 guide RNA |
| 502 | Shortened/unmodified NmeCas9 guide RNA |
| 503 | Shortened/unmodified NmeCas9 guide RNA |
| 504 | Mod-N77 conserved portion only |
| 505 | Mod-N78 conserved portion only |
| 506 | Shortened/unmodified NmeCas9 guide RNA comprising internal linkers |
| 507 | Shortened/modified NmeCas9 guide RNA comprising internal linkers |
| 508 | Shortened/modified NmeCas9 guide RNA |
| 509-535 | Exemplary sgRNAs |

Transcript sequences may generally include GGG as the first three nucleotides for use with ARCA or AGG as the first three nucleotides for use with CleanCap™. Accordingly, the first three nucleotides can be modified for use with other capping approaches, such as Vaccinia capping enzyme. Promoters and poly-A sequences are not included in the transcript sequences. A promoter such as a U6 promoter (SEQ ID NO: 389) or a CMV Promotor (SEQ ID NO: 390) and a poly-A sequence such as SEQ ID NO: 409 can be appended to the disclosed transcript sequences at the 5′ and 3′ ends, respectively. Most nucleotide sequences are provided as DNA but can be readily converted to RNA by changing Ts to Us.

### DETAILED DESCRIPTION OF CERTAIN EMBODIMENTS

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The term “about” is used herein to mean within the typical ranges of tolerances in the art. For example, “about” can be understood as about 2 standard deviations from the mean. In certain embodiments, about means +10%. In certain embodiments, about means +5%, +2%, or +1%. When about is present before a series of numbers or a range, it is understood that “about” can modify each of the numbers in the series or range. At the very least, and not as an attempt to limit the application of the doctrine of equivalents to the scope of the claims, each numerical parameter should at least be construed in light of the number of reported significant digits and by applying ordinary rounding techniques.

The use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has 2, 1, or 0 nucleotide base pairs. When “no more than” or “less than” is present before a series of numbers or a range, it is understood that each of the numbers in the series or range is modified.

As used herein, ranges include both the upper and lower limits.

As used herein, it is understood that when the maximum amount of a value is represented by 100% (e.g., 100% inhibition) that the value is interpreted in light of the method of detection. For example, 100% inhibition, and the like, is understood as inhibition to a level below the level of detection of the assay.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts the express content of this specification, including but not limited to a definition, the express content of this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABC, CBBA, BABB, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

“Or” is used in the inclusive sense, i.e., equivalent to “and/or,” unless the context requires otherwise.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941) and is included herein as SEQ ID NO: 151. In some embodiments, the APOBEC3A protein is a human APOBEC3A protein or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (Nme1Cas9, Nme2Cas9, and Nme3Cas9). The Nme2Cas9 ortholog functions efficiently in mammalian cells, recognizes an N4CC PAM, and can be used for in vivo editing (Ran et al., NATURE, vol. 520, 2015, pages 186-191; Kim et al., NAT. COMMUN., vol. 8, 2017, pages 14500). Nme2Cas9 has been shown to be naturally resistant to off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Further, NmeCas9 variants are known in the art, see, e.g., Huang et al., Nature Biotech. 2022, doi.org/10.1038/s41587-022-01410-2, which describes Cas9 variants targeting single-nucleotide-pyrimidine PAMs. Throughout, “NmeCas9” or “Nine Cas9” is generic and an encompasses any type of NmeCas9, including, Nme1Cas9, Nme2Cas9, and Nme3Cas9.

As used herein, the term “fusion protein” refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

As used herein, the term “uracil glycosylase inhibitor”, “uracil-DNA glycosylase inhibitor” or “UGI” refers to a protein that is capable of inhibiting a uracil-DNA glycosylase (UDG) base-excision repair enzyme (e.g., UniProt ID: P14739; SEQ ID NO: 3).

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein). Exemplary peptide linkers are disclosed elsewhere herein.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine, e.g., N1-methyl-psuedouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g., via fusion with a FokI domain. Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, preferably 90%, 95%, 96%, 97%, 98%, or 99% identity to any of the Cas9 nucleic acid sequences, amino acid sequences, or nucleic acid sequences encoding the amino acid sequences provided herein are also contemplated. Exemplary open reading frame for Cas9 are provided in Table 39A.

As used herein, the “minimal uridine codon(s)” for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating uridine content.

As used herein, the “uridine dinucleotide (UU) content” of an ORF can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating uridine dinucleotide content.

As used herein, the “minimal adenine codon(s)” for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating adenine content.

As used herein, the “adenine dinucleotide content” of an ORF can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating adenine dinucleotide content.

As used herein, the “minimum repeat content” of a given open reading frame (ORF) is the minimum possible sum of occurrences of AA, CC, GG, and TT (or TU, UT, or UU) dinucleotides in an ORF that encodes the same amino acid sequence as the given ORF. The repeat content can be expressed in absolute terms as the enumeration of AA, CC, GG, and TT (or TU, UT, or UU) dinucleotides in an ORF or on a rate basis as the enumeration of AA, CC, GG, and TT (or TU, UT, or UU) dinucleotides in an ORF divided by the length in nucleotides of the ORF (for example, UAAUA would have a repeat content of 20% because one repeat occurs in a sequence of 5 nucleotides). Modified adenine, guanine, cytosine, thymine, and uracil residues are considered equivalent to adenine, guanine, cytosine, thymine, and uracil residues for the purpose of evaluating minimum repeat content.

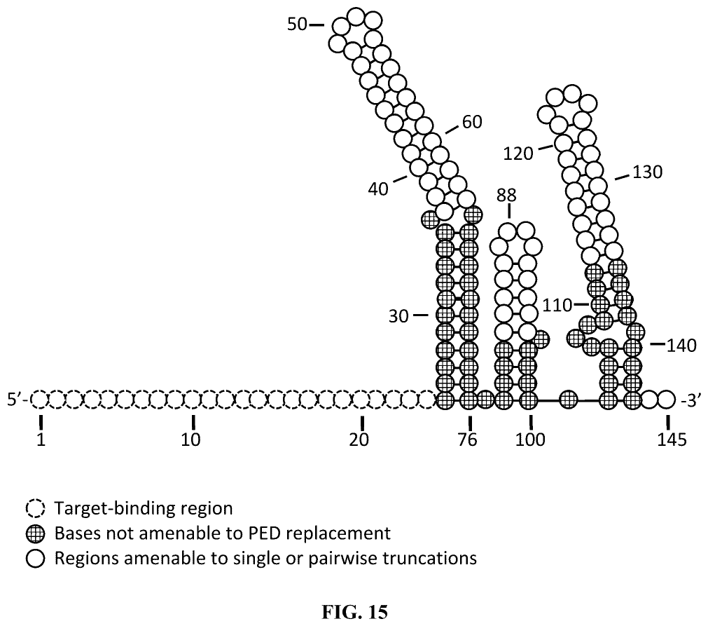

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally occurring sequences. Guide RNAs can include modified RNAs as described herein. Unless otherwise clear from the context, guide RNAs described herein are suitable for use with an Nine Cas9, e.g., an Nme1, Nme2, or Nme3 Cas9. For example, FIG. 15 shows an exemplary schematic of Nme2 sgRNA in a possible secondary structure.

As used herein, a “guide sequence” or “guide region” or “spacer” or “spacer sequence” and the like refers to a sequence within a guide RNA that is complementary to a target sequence and functions to direct a guide RNA to a target sequence for binding or modification (e.g., cleavage) by NmeCas9. A guide sequence can be 20-25 nucleotides in length, e.g., in the case of Nine Cas9 and related Cas9 homologs/orthologs. Shorter or longer sequences can also be used as guides, e.g., 20-, 21-, 22-, 23-, 24-, or 25-nucleotides in length. A guide sequence can be at least 22-, 23-, 24-, or 25-nucleotides in length in the case of Nine Cas9. A guide sequence can form a 22-, 23-, 24, or 25-continuous base pair duplex, e.g., a 24-continuous base pair duplex, with its target sequence in the case of Nine Cas9.

Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “indels” refer to insertion/deletion mutations consisting of a number of nucleotides that are either inserted or deleted at the site of double-stranded breaks (DSBs) in the nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” a target protein one or more cells (e.g., in a population of cells including in vivo populations such as those found in tissues). In some embodiments, a knockout is not the formation of mutant of the target protein, for example, created by indels, but rather the complete loss of expression of the target protein in a cell.

As used herein, “ribonucleoprotein” (RNP) or “RNP complex” refers to a guide RNA together with an RNA-guided DNA binding agent, such as a Cas cleavase, nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Nine Cas9 protein or Nine Cas9 ortholog (Edraki et al., 2019). In some embodiments, the PAM may comprise 2, 3, 4, 5, 6, 7, 8, 9, or 10 nucleotides in length. Non-limiting exemplary PAM sequences include NCC, N4GAYW, N4GYTT, N4GTCT, NNNNCC(a), NNNNCAAA (wherein N is defined as any nucleotide, W is defined as either A or T, and R is defined as either A or G; and (a) is a preferred, but not required, A after the second C)). In some embodiments, the PAM sequence may be NCC.

As used herein, “treatment” refers to any administration or application of a therapeutic for disease or disorder in a subject, and includes slowing or arresting disease development or progression, relieving one or more signs or symptoms of the disease, curing the disease, or preventing reoccurrence of one or more symptoms of the disease.

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local or topical delivery. See also, e.g., WO2017173054A1, the contents of which are hereby incorporated by reference in their entirety. Any LNP known to those of skill in the art to be capable of delivering nucleotides to subjects may be utilized with the guide RNAs and the nucleic acid encoding an RNA-guided DNA binding agent described herein.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be linked to the remaining parts of the molecule by covalent bonds, hydrogen bonds or ionic interactions.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

### II. Exemplary Polynucleotides and Compositions

In some embodiments, a polynucleotide is provided, the polynucleotide comprising an open reading frame (ORF), the ORF comprising:
a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321; and
a nucleotide sequence encoding a first nuclear localization signal (NLS); and In some embodiments, the Nine Cas9 is an Nme2 Cas9. In some embodiments, the Nine Cas9 is an Nme1 Cas9. In some embodiments, the Nine Cas9 is an Nme3 Cas9. In some embodiments, the ORF further comprises a nucleotide sequence encoding a second NLS. In some embodiments, the polynucleotide is an mRNA.

In some embodiments, the ORF comprises a sequence having at least 90%, 95%, 98% or 100% identity to the sequence of any one of SEQ ID NO: 29, 32-41, 224-226, 231-233, 238-240, 245-247, 252-254, 259-261, 266-268, 273-275, 280-282, 287-289, 294-296, 301-303, or 316-321. In some embodiments, the ORF comprises a sequence having at least 90%, 95%, 98% or 100% identity to the sequence of any one of SEQ ID NO: 29 or 32-41. In some embodiments, the ORF comprises a sequence having at least 90%, 95%, 98% or 100% identity to the sequence of SEQ ID NO: 32. In some embodiments, the ORF comprises a sequence having at least 90%, 95%, 98% or 100% identity to the sequence of SEQ ID NO: 33. In some embodiments, the ORF comprises a sequence having at least 90%, 95%, 98% or 100% identity to the sequence of SEQ ID NO: 34. In some embodiments, the ORF comprises a sequence having at least 90%, 95%, 98% or 100% identity to the sequence of SEQ ID NO: 35. In some embodiments, the ORF comprises a sequence having at least 90%, 95%, 98% or 100% identity to the sequence of SEQ ID NO: 36. In some embodiments, the ORF comprises a sequence having at least 90%, 95%, 98% or 100% identity to the sequence of SEQ ID NO: 38. In some embodiments, the ORF comprises a sequence having at least 90%, 95%, 98% or 100% identity to the sequence of SEQ ID NO: 39. In some embodiments, the ORF comprises a sequence having at least 90%, 95%, 98% or 100% identity to the sequence of SEQ ID NO: 41.

In some embodiments, the ORF comprises a sequence having at least 90%, 95%, 98% or 100% identity to the sequence of SEQ ID NO: 38 or 41.

In some embodiments, a polynucleotide is provided, the polynucleotide comprising the ORF disclosed herein. In some embodiments, a polynucleotide is provided, the polynucleotide encoding an Nine Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321, wherein the Nine Cas9 is an Nme2 Cas9, Nme3 Cas9, or an Nme1 Cas9, a first nuclear localization signal (NLS); and a second NLS, wherein the encoded first NLS and the second NLS are located to N-terminal to the NmeCas9 polypeptide.

In some embodiments, a polypeptide is provided, the polypeptide comprising an Nine Cas9 polypeptide at least 90% identical to any one of an amino acid sequence with at least 91%, 92%, 93%, 94%, 95%, 96%, 97%, 98%, or 99% to any one of SEQ ID NOs: 1 and 4-13, 220, 227, 234, 241, 248, 255, 262, 269, 276, 283, 290, or 297, or 310-315, wherein the Nine Cas9 is an Nme2 Cas9, Nme3 Cas9, or an Nme1 Cas9, a first nuclear localization signal (NLS); and a second NLS, wherein the encoded first NLS and the second NLS are located to N-terminal to the NmeCas9 polypeptide.

In some embodiments, methods of modifying a target gene are provided comprising administering the compositions described herein. In some embodiments, the method comprises delivering to a cell a polynucleotide comprising an open reading frame (ORF), the ORF comprising: a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321, wherein the Nine Cas9 is an Nme2 Cas9 or an Nme1 Cas9 or an Nine 3 Cas9; a nucleotide sequence encoding a first nuclear localization signal (NLS); and optionally a nucleotide sequence encoding a second NLS. In some embodiments, the polynucleotide is delivered to a cell in vitro. In some embodiments, the polynucleotide is delivered to a cell in vivo.

In some embodiments, the composition described herein further comprises at least one gRNA. In some embodiments, a composition is provided that comprises an mRNA described herein and at least one gRNA. In some embodiments, the gRNA is a single guide RNA (sgRNA). In some embodiments, the gRNA is a dual guide RNA (dgRNA).

In some embodiments, the composition is capable of effecting genome editing upon administration to a subject. In some embodiments, the subject is a human.

### A. RNA-Guided DNA Binding Agent; NmeCas9

RNA-guided DNA binding agents described herein encompass Neisseria meningitidis Cas9 (NmeCas9) and modified and variants thereof. In some embodiments, the NmeCas9 is Nme2 Cas9. In some embodiments, the NmeCas9 is Nme1 Cas9. In some embodiments, the NmeCas9 is Nme3 Cas9.

Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas, e.g., a nickase Cas9, that induces a nick rather than a double strand break in the target DNA.

In some embodiments, the NmeCas9 nuclease may be modified to contain only one functional nuclease domain. For example, the RNA-guided DNA binding agent may be modified such that one of the nuclease domains is mutated or fully or partially deleted to reduce its nucleic acid cleavage activity.

In some embodiments, a NmeCas9 nickase is used having a RuvC domain with reduced activity. In some embodiments, a NmeCas9 nickase is used having an inactive RuvC domain. In some embodiments, a NmeCas9 nickase is used having an HNH domain with reduced activity. In some embodiments, a NmeCas9 nickase is used having an inactive HNH domain.

In some embodiments, a conserved amino acid within a NmeCas9 nuclease domain is substituted to reduce or alter nuclease activity. Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain or more than one HNH domain. In some embodiments, the Cas9 nuclease is a wild type Cas9. In some embodiments, the Cas9 is capable of inducing a double strand break in target DNA. In certain embodiments, the Cas nuclease may cleave dsDNA, it may cleave one strand of dsDNA, or it may not have DNA cleavase or nickase activity. In some embodiments, a NmeCas9 may comprise an amino acid substitution in the RuvC or RuvC-like nuclease domain. Exemplary amino acid substitutions in the RuvC or RuvC-like nuclease domain include H588A (based on the N. meningitidis Cas9 protein). In some embodiments, the Cas protein may comprise an amino acid substitution in the HNH or HNH-like nuclease domain. Exemplary amino acid substitutions in the HNH or HNH-like nuclease domain include D16A (based on the NmeCas9 protein).

In some embodiments, chimeric Cas proteins are used, where one domain or region of the protein is replaced by a portion of a different protein. In some embodiments, a NmeCas9 nuclease domain may be replaced with a domain from a different nuclease such as Fok1. In some embodiments, a NmeCas9 protein may be a modified NmeCas9 nuclease.

In some embodiments, the nuclease may be modified to induce a point mutation or base change, e.g., a deamination.

In some embodiments, the Cas protein comprises a fusion protein comprising a Cas nuclease (e.g., NmeCas9), which is a nickase or is catalytically inactive, linked to a heterologous functional domain. In some embodiments, the Cas protein comprises a fusion protein comprising a catalytically inactive Cas nuclease (e.g., NmeCas9) linked to a heterologous functional domain (see, e.g., WO2014152432). In some embodiments, the catalytically inactive Cas9 is from the N. meningitidis Cas9. In some embodiments, the catalytically inactive Cas comprises mutations that inactivate the Cas.

In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase. A heterologous functional domain such as a deaminase may be part of a fusion protein with a Cas nuclease having nickase activity or a Cas nuclease that is catalytically inactive discussed further below.

In some embodiments, the Nine Cas9 has double stranded endonuclease activity.

In some embodiments, the Nine Cas9 has nickase activity.

In some embodiments, the Nine Cas9 comprises a dCas9 DNA binding domain.

In some embodiments, the Nine Cas9 comprises an amino acid sequence at least 91%, 92%, 93%, 94%, 95%, 96%, 97%, 98%, or 99% identical to any one of SEQ ID NOs: 1 and 4-13, 220, 227, 234, 241, 248, 255, 262, 269, 276, 283, 290, or 297, or 310-315 (as shown in Table 39A). In some embodiments, the Nine Cas9 comprises an amino acid sequence of any one of SEQ ID NOs: 1 and 4-13, 220, 227, 234, 241, 248, 255, 262, 269, 276, 283, 290, 297, or 310-315 (as shown in Table 39A).

In some embodiments, the sequence encoding the NmeCas9 comprises a nucleotide sequence at least 91%, 92%, 93%, 94%, 95%, 96%, 97%, 98%, or 99% identical to any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321 (as shown in Table 39A). In some embodiments, the sequence encoding the NmeCas9 comprises a nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321 (as shown in Table 39A).

In some embodiments, any of the foregoing levels of identity is at least 95%, at least 98%, at least 99%, or 100%.

### B. Exemplary Coding Sequences

In any of the embodiments set forth herein, the polynucleotide is a mRNA comprising an ORF encoding an RNA-guided DNA binding agent disclosed above. In any of the embodiments set forth herein, the polynucleotide is a mRNA comprising an ORF encoding an NmeCas9. In any of the embodiments set forth herein, the polynucleotide may be an expression construct comprising a promoter operably linked to an ORF encoding an RNA-guided DNA binding agent (e.g., NmeCas9).

Certain ORFs are translated in vivo more efficiently than others in terms of polypeptide molecules produced per mRNA molecule. The codon pair usage of such efficiently translated ORFs may contribute to translation efficiency. Further description of improvement of ORF coding sequence, codon pair usage, codon repeat contents are disclosed in WO 2019/0067910 and WO 2020/198641, the contents of each of which are hereby incorporated by reference in their entirety.

For example, in some embodiments, at least 75%, 80%, 85%, 90%, 95%, 98%, 99%, or 100% of the codons of the ORF are minimal adenine codons or minimal uridine codons. In some embodiments, the ORF comprises or consists of codons that increase translation of the mRNA in a mammal. In some embodiments, the ORF comprises or consists of codons that increase translation of the mRNA in a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level.

In some embodiments, the GC content of the ORF is greater than or equal to 56%. In some embodiments, the GC content of the ORF is greater than or equal to 56.5%. In some embodiments, the GC content of the ORF is greater than or equal to 57%. In some embodiments, the GC content of the ORF is greater than or equal to 57.5%. In some embodiments, the GC content of the ORF is greater than or equal to 58%. In some embodiments, the GC content of the ORF is greater than or equal to 58.5%. In some embodiments, the GC content of the ORF is greater than or equal to 59%. In some embodiments, the GC content of the ORF is less than or equal to 63%. In some embodiments, the GC content of the ORF is less than or equal to 62.6%. In some embodiments, the GC content of the ORF is less than or equal to 62.1%. In some embodiments, the GC content of the ORF is less than or equal to 61.6%. In some embodiments, the GC content of the ORF is less than or equal to 61.1%. In some embodiments, the GC content of the ORF is less than or equal to 60.6%. In some embodiments, the GC content of the ORF is less than or equal to 60.1%.

In some embodiments, the ORF consists of a set of codons of which at least about 75%, 80%, 85%, 90%, 95%, 98%, 99%, or 100% of the codons are codons listed in Table 1.

TABLE 1

| Amino Acid | Low A/U |
|---|---|
| Gly | GGC |
| Glu | GAG |
| Asp | GAC |
| Val | GTG |
| Ala | GCC |
| Arg | CGG |
| Ser | AGC |
| Lys | AAG |
| Asn | AAC |
| Met | ATG |
| Ile | ATC |
| Thr | ACC |
| Trp | TGG |
| Cys | TGC |
| Tyr | TAC |
| Leu | CTG |
| Phe | TTC |
| Gln | CAG |
| His | CAC |

1. ORFs with Low Uridine Content

In some embodiments, the ORF encoding a polypeptide has a uridine content ranging from its minimum uridine content to about 150% of its minimum uridine content. In some embodiments, the uridine content of the ORF is less than or equal to about 145%, 140%, 135%, 130%, 125%, 120%, 115%, 110%, 105%, 104%, 103%, 102%, or 101% of its minimum uridine content. In some embodiments, the ORF has a uridine content equal to its minimum uridine content. In some embodiments, the ORF has having a uridine content less than or equal to about 150% of its minimum uridine content. In some embodiments, the ORF has a uridine content less than or equal to about 145% of its minimum uridine content. In some embodiments, the ORF has a uridine content less than or equal to about 140% of its minimum uridine content. In some embodiments, the ORF has a uridine content less than or equal to about 135% of its minimum uridine content. In some embodiments, the ORF has a uridine content less than or equal to about 130% of its minimum uridine content. In some embodiments, the ORF has a uridine content less than or equal to about 125% of its minimum uridine content. In some embodiments, the ORF has a uridine content less than or equal to about 120% of its minimum uridine content. In some embodiments, the ORF has a uridine content less than or equal to about 115% of its minimum uridine content. In some embodiments, the ORF has a uridine content less than or equal to about 110% of its minimum uridine content. In some embodiments, the ORF has a uridine content less than or equal to about 105% of its minimum uridine content. In some embodiments, the ORF has a uridine content less than or equal to about 104% of its minimum uridine content. In some embodiments, the ORF has a uridine content less than or equal to about 103% of its minimum uridine content. In some embodiments, the ORF has a uridine content less than or equal to about 102% of its minimum uridine content. In some embodiments, the ORF has a uridine content less than or equal to about 101% of its minimum uridine content.

In some embodiments, the ORF has a uridine dinucleotide content ranging from its minimum uridine dinucleotide content to 200% of its minimum uridine dinucleotide content. In some embodiments, the uridine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum uridine dinucleotide content. In some embodiments, the ORF has a uridine dinucleotide content equal to its minimum uridine dinucleotide content. In some embodiments, the ORF has a uridine dinucleotide content less than or equal to about 200% of its minimum uridine dinucleotide content. In some embodiments, the ORF has a uridine dinucleotide content less than or equal to about 195% of its minimum uridine dinucleotide content. In some embodiments, the ORF has a uridine dinucleotide content less than or equal to about 190% of its minimum uridine dinucleotide content. In some embodiments, the ORF has a uridine dinucleotide content less than or equal to about 185% of its minimum uridine dinucleotide content. In some embodiments, the ORF has a uridine dinucleotide content less than or equal to about 180% of its minimum uridine dinucleotide content. In some embodiments, the ORF has a uridine dinucleotide content less than or equal to about 175% of its minimum uridine dinucleotide content. In some embodiments, the ORF has a uridine dinucleotide content less than or equal to about 170% of its minimum uridine dinucleotide content. In some embodiments, the ORF has a uridine dinucleotide content less than or equal to about 165% of its minimum uridine dinucleotide content. In some embodiments, the ORF has a uridine dinucleotide content less than or equal to about 160% of its minimum uridine dinucleotide content. In some embodiments, the ORF has a uridine dinucleotide content less than or equal to about 155% of its minimum uridine dinucleotide content. In some embodiments, the ORF has a uridine dinucleotide content equal to its minimum uridine dinucleotide content. In some embodiments, the ORF has a uridine dinucleotide content less than or equal to about 150% of its minimum uridine dinucleotide content. In some embodiments, the ORF has a uridine dinucleotide content less than or equal to about 145% of its minimum uridine dinucleotide content. In some embodiments, the ORF has a uridine dinucleotide content less than or equal to about 140% of its minimum uridine dinucleotide content. In some embodiments, the ORF has a uridine dinucleotide content less than or equal to about 135% of its minimum uridine dinucleotide content. In some embodiments, the ORF has a uridine dinucleotide content less than or equal to about 130% of its minimum uridine dinucleotide content. In some embodiments, the ORF has a uridine dinucleotide content less than or equal to about 125% of its minimum uridine dinucleotide content. In some embodiments, the ORF has a uridine dinucleotide content less than or equal to about 120% of its minimum uridine dinucleotide content. In some embodiments, the ORF has a uridine dinucleotide content less than or equal to about 115% of its minimum uridine dinucleotide content. In some embodiments, the ORF has a uridine dinucleotide content less than or equal to about 110% of its minimum uridine dinucleotide content. In some embodiments, the ORF has a uridine dinucleotide content less than or equal to about 105% of its minimum uridine dinucleotide content. In some embodiments, the ORF has a uridine dinucleotide content less than or equal to about 104% of its minimum uridine dinucleotide content. In some embodiments, the ORF has a uridine dinucleotide content less than or equal to about 103% of its minimum uridine dinucleotide content. In some embodiments, the ORF has a uridine dinucleotide content less than or equal to about 102% of its minimum uridine dinucleotide content. In some embodiments, the ORF has a uridine dinucleotide content less than or equal to about 101% of its minimum uridine dinucleotide content.

In some embodiments, the ORF has a uridine dinucleotide content ranging from its minimum uridine dinucleotide content to the uridine dinucleotide content that is 90% or lower of the maximum uridine dinucleotide content of a reference sequence that encodes the same protein as the mRNA in question. In some embodiments, the uridine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine dinucleotide content of a reference sequence that encodes the same protein as the mRNA in question.

In some embodiments, the ORF has a uridine trinucleotide content ranging from 0 uridine trinucleotides to 1, 2, 3, 4, 5, 6, 7, 8, 9, 10, 20, 30, 40, or 50 uridine trinucleotides (where a longer run of uridines counts as the number of unique three-uridine segments within it, e.g., a uridine tetranucleotide contains two uridine trinucleotides, a uridine pentanucleotide contains three uridine trinucleotides, etc.). In some embodiments, the ORF has a uridine trinucleotide content ranging from 0% uridine trinucleotides to 0.1%, 0.2%, 0.3%, 0.4%, 0.5%, 0.6%, 0.7%, 0.8%, 0.9%, 1%, 1.5%, or 2% uridine trinucleotides, where the percentage content of uridine trinucleotides is calculated as the percentage of positions in a sequence that are occupied by uridines that form part of a uridine trinucleotide (or longer run of uridines), such that the sequences UUUAAA and UUUUAAAA would each have a uridine trinucleotide content of 50%. For example, in some embodiments, the ORF has a uridine trinucleotide content less than or equal to 2%. For example, in some embodiments, the ORF has a uridine trinucleotide content less than or equal to 1.5%. In some embodiments, the ORF has a uridine trinucleotide content less than or equal to 1%. In some embodiments, the ORF has a uridine trinucleotide content less than or equal to 0.9%. In some embodiments, the ORF has a uridine trinucleotide content less than or equal to 0.8%. In some embodiments, the ORF has a uridine trinucleotide content less than or equal to 0.7%. In some embodiments, the ORF has a uridine trinucleotide content less than or equal to 0.6%. In some embodiments, the ORF has a uridine trinucleotide content less than or equal to 0.5%. In some embodiments, the ORF has a uridine trinucleotide content less than or equal to 0.4%. In some embodiments, the ORF has a uridine trinucleotide content less than or equal to 0.3%. In some embodiments, the ORF has a uridine trinucleotide content less than or equal to 0.2%. In some embodiments, the ORF has a uridine trinucleotide content less than or equal to 0.10%. In some embodiments, the ORF has no uridine trinucleotides.

In some embodiments, the ORF has a uridine trinucleotide content ranging from its minimum uridine trinucleotide content to the uridine trinucleotide content that is 90% or lower of the maximum uridine trinucleotide content of a reference sequence that encodes the same protein as the polynucleotide in question. In some embodiments, the uridine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine trinucleotide content of a reference sequence that encodes the same protein as the polynucleotide in question.

In some embodiments, the ORF has minimal nucleotide homopolymers, e.g., repetitive strings of the same nucleotides. For example, in some embodiments, when selecting a minimal uridine codon from the codons listed in Table 2, a polynucleotide is constructed by selecting the minimal uridine codons that reduce the number and length of nucleotide homopolymers, e.g., selecting GCA instead of GCC for alanine or selecting GGA instead of GGG for glycine or selecting AAG instead of AAA for lysine.

A given ORF can be reduced in uridine content or uridine dinucleotide content or uridine trinucleotide content, for example, by using minimal uridine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal uridine codons shown below. In some embodiments, at least about 50%, 55%, 60%, 65%, 70%, 75%, 80%, 85%, 90%, 95%, 98%, 99%, or 100% of the codons in the ORF are codons listed in Table 2.

TABLE 2
Exemplary minimal uridine codons

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 2.

2. ORFs with Low Adenine Content

In some embodiments, the ORF has an adenine content ranging from its minimum adenine content to about 150% of its minimum adenine content. In some embodiments, the adenine content of the ORF is less than or equal to about 145%, 140%, 135%, 130%, 125%, 120%, 115%, 110%, 105%, 104%, 103%, 102%, or 101% of its minimum adenine content. In some embodiments, the ORF has an adenine content equal to its minimum adenine content. In some embodiments, the ORF has an adenine content less than or equal to about 150% of its minimum adenine content. In some embodiments, the ORF has an adenine content less than or equal to about 145% of its minimum adenine content. In some embodiments, the ORF has an adenine content less than or equal to about 140% of its minimum adenine content. In some embodiments, the ORF has an adenine content less than or equal to about 135% of its minimum adenine content. In some embodiments, the ORF has an adenine content less than or equal to about 130% of its minimum adenine content. In some embodiments, the ORF has an adenine content less than or equal to about 125% of its minimum adenine content. In some embodiments, the ORF has an adenine content less than or equal to about 120% of its minimum adenine content. In some embodiments, the ORF has an adenine content less than or equal to about 115% of its minimum adenine content. In some embodiments, the ORF has an adenine content less than or equal to about 110% of its minimum adenine content. In some embodiments the ORF has an adenine content less than or equal to about 105% of its minimum adenine content. In some embodiments, the ORF has an adenine content less than or equal to about 104% of its minimum adenine content. In some embodiments, the ORF has an adenine content less than or equal to about 103% of its minimum adenine content. In some embodiments, the ORF has an adenine content less than or equal to about 102% of its minimum adenine content. In some embodiments, the ORF has an adenine content less than or equal to about 101% of its minimum adenine content.

In some embodiments, the ORF has an adenine dinucleotide content ranging from its minimum adenine dinucleotide content to 200% of its minimum adenine dinucleotide content. In some embodiments, the adenine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum adenine dinucleotide content. In some embodiments, the ORF has an adenine dinucleotide content equal to its minimum adenine dinucleotide content. In some embodiments, the ORF has an adenine dinucleotide content less than or equal to about 200% of its minimum adenine dinucleotide content. In some embodiments, the ORF has an adenine dinucleotide content less than or equal to about 195% of its minimum adenine dinucleotide content. In some embodiments, the ORF has an adenine dinucleotide content less than or equal to about 190% of its minimum adenine dinucleotide content. In some embodiments, the ORF has an adenine dinucleotide content less than or equal to about 185% of its minimum adenine dinucleotide content. In some embodiments, the ORF has an adenine dinucleotide content less than or equal to about 180% of its minimum adenine dinucleotide content. In some embodiments, the ORF has an adenine dinucleotide content less than or equal to about 175% of its minimum adenine dinucleotide content. In some embodiments, the ORF has an adenine dinucleotide content less than or equal to about 170% of its minimum adenine dinucleotide content. In some embodiments, the ORF has an adenine dinucleotide content less than or equal to about 165% of its minimum adenine dinucleotide content. In some embodiments, the ORF has an adenine dinucleotide content less than or equal to about 160% of its minimum adenine dinucleotide content. In some embodiments, the ORF has an adenine dinucleotide content less than or equal to about 155% of its minimum adenine dinucleotide content. In some embodiments, the ORF has an adenine dinucleotide content equal to its minimum adenine dinucleotide content. In some embodiments, the ORF has an adenine dinucleotide content less than or equal to about 150% of its minimum adenine dinucleotide content. In some embodiments, the ORF has an adenine dinucleotide content less than or equal to about 145% of its minimum adenine dinucleotide content. In some embodiments, the ORF has an adenine dinucleotide content less than or equal to about 140% of its minimum adenine dinucleotide content. In some embodiments, the ORF has an adenine dinucleotide content less than or equal to about 135% of its minimum adenine dinucleotide content. In some embodiments, the ORF has an adenine dinucleotide content less than or equal to about 130% of its minimum adenine dinucleotide content. In some embodiments, the ORF has an adenine dinucleotide content less than or equal to about 125% of its minimum adenine dinucleotide content. In some embodiments, the ORF has an adenine dinucleotide content less than or equal to about 120% of its minimum adenine dinucleotide content. In some embodiments, the ORF has an adenine dinucleotide content less than or equal to about 115% of its minimum adenine dinucleotide content. In some embodiments, the ORF has an adenine dinucleotide content less than or equal to about 110% of its minimum adenine dinucleotide content. In some embodiments, the ORF has an adenine dinucleotide content less than or equal to about 105% of its minimum adenine dinucleotide content. In some embodiments, the ORF has an adenine dinucleotide content less than or equal to about 104% of its minimum adenine dinucleotide content. In some embodiments, the ORF has an adenine dinucleotide content less than or equal to about 103% of its minimum adenine dinucleotide content. In some embodiments, the ORF has an adenine dinucleotide content less than or equal to about 102% of its minimum adenine dinucleotide content. In some embodiments, the ORF has an adenine dinucleotide content less than or equal to about 101% of its minimum adenine dinucleotide content.

In some embodiments, the ORF has an adenine dinucleotide content ranging from its minimum adenine dinucleotide content to the adenine dinucleotide content that is 90% or lower of the maximum adenine dinucleotide content of a reference sequence that encodes the same protein as the polynucleotide in question. In some embodiments, the adenine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine dinucleotide content of a reference sequence that encodes the same protein as the polynucleotide in question.

In some embodiments, the ORF has an adenine trinucleotide content ranging from 0 adenine trinucleotides to 1, 2, 3, 4, 5, 6, 7, 8, 9, 10, 20, 30, 40, or 50 adenine trinucleotides (where a longer run of adenines counts as the number of unique three-adenine segments within it, e.g., an adenine tetranucleotide contains two adenine trinucleotides, an adenine pentanucleotide contains three adenine trinucleotides, etc.). In some embodiments, the ORF has an adenine trinucleotide content ranging from 0% adenine trinucleotides to 0.1%, 0.2%, 0.3%, 0.4%, 0.5%, 0.6%, 0.7%, 0.8%, 0.9%, 1%, 1.5%, or 2% adenine trinucleotides, where the percentage content of adenine trinucleotides is calculated as the percentage of positions in a sequence that are occupied by adenines that form part of an adenine trinucleotide (or longer run of adenines), such that the sequences UUUAAA and UUUUAAAA would each have an adenine trinucleotide content of 50%. For example, in some embodiments, the ORF has an adenine trinucleotide content less than or equal to 2%. For example, in some embodiments, the ORF has an adenine trinucleotide content less than or equal to 1.5%. In some embodiments, the ORF has an adenine trinucleotide content less than or equal to 1%. In some embodiments, the ORF has an adenine trinucleotide content less than or equal to 0.9%. In some embodiments, the ORF has an adenine trinucleotide content less than or equal to 0.8%. In some embodiments, the ORF has an adenine trinucleotide content less than or equal to 0.7%. In some embodiments, the ORF has an adenine trinucleotide content less than or equal to 0.6%. In some embodiments, the ORF has an adenine trinucleotide content less than or equal to 0.5%. In some embodiments, the ORF has an adenine trinucleotide content less than or equal to 0.4%. In some embodiments, the ORF has an adenine trinucleotide content less than or equal to 0.3%. In some embodiments, the ORF has an adenine trinucleotide content less than or equal to 0.2%. In some embodiments, the ORF has an adenine trinucleotide content less than or equal to 0.1%. In some embodiments, the ORF has no adenine trinucleotides.

In some embodiments, the ORF has an adenine trinucleotide content ranging from its minimum adenine trinucleotide content to the adenine trinucleotide content that is 90% or lower of the maximum adenine trinucleotide content of a reference sequence that encodes the same protein as the polynucleotide in question. In some embodiments, the adenine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine trinucleotide content of a reference sequence that encodes the same protein as the polynucleotide in question. In some embodiments, the ORF has minimal nucleotide homopolymers, e.g., repetitive strings of the same nucleotides. For example, in some embodiments, when selecting a minimal adenine codon from the codons listed in Table 3, a polynucleotide is constructed by selecting the minimal adenine codons that reduce the number and length of nucleotide homopolymers, e.g., selecting GCA instead of GCC for alanine or selecting GGA instead of GGG for glycine or selecting AAG instead of AAA for lysine. A given ORF can be reduced in adenine content or adenine dinucleotide content or adenine trinucleotide content, for example, by using minimal adenine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 3.

TABLE 3
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 3.

3. ORFs with Low Adenine and Low Uridine Content

To the extent feasible, any of the features described above with respect to low adenine content can be combined with any of the features described above with respect to low uridine content. For example, the ORF has a uridine content ranging from its minimum uridine content to about 150% of its minimum uridine content (e.g., a uridine content of the ORF is less than or equal to about 145%, 140%, 135%, 130%, 125%, 120%, 115%, 110%, 105%, 104%, 103%, 102%, or 101% of its minimum uridine content) and an adenine content ranging from its minimum adenine content to about 150% of its minimum adenine content (e.g., less than or equal to about 145%, 140%, 135%, 130%, 125%, 120%, 115%, 110%, 105%, 104%, 103%, 102%, or 101% of its minimum adenine content). So too for uridine and adenine dinucleotides. Similarly, the content of uridine nucleotides and adenine dinucleotides in the ORF may be as set forth above. Similarly, the content of uridine dinucleotides and adenine nucleotides in the ORF may be as set forth above.

A given ORF can be reduced in uridine and adenine nucleotide or dinucleotide content, for example, by using minimal uridine and adenine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal uridine and adenine codons shown below. In some embodiments, at least about 50%, 55%, 60%, 65%, 70%, 75%, 80%, 85%, 90%, 95%, 98%, 99%, or 100% of the codons in the ORF are codons listed in Table 4.

TABLE 4
Exemplary minimal uridine and adenine codons

| | Amino Acid | Minimal uridine codon |
|---|---|---|
| A | Alanine | GCC or GCG |
| G | Glycine | GGC or GGG |
| V | Valine | GUC or GUG |
| D | Aspartic acid | GAC |
| E | Glutamic acid | GAG |
| I | Isoleucine | AUC |
| T | Threonine | ACC or ACG |
| N | Asparagine | AAC |
| K | Lysine | AAG |
| S | Serine | AGC or UCC or UCG |
| R | Arginine | CGC or CGG |
| L | Leucine | CUG or CUC |
| P | Proline | CCG or CCC |
| H | Histidine | CAC |
| Q | Glutamine | CAG |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 4. As can be seen in Table 4, each of the three listed serine codons contains either one A or one U. In some embodiments, uridine minimization is prioritized by using AGC codons for serine. In some embodiments, adenine minimization is prioritized by using UCC or UCG codons for serine.

4. Codons that Increase Translation or that Correspond to Highly Expressed tRNAs; Exemplary Codon Sets

In some embodiments, the ORF has codons that increase translation in a mammal, such as a human. In further embodiments, the ORF has codons that increase translation in an organ, such as the liver, of the mammal, e.g., a human. In further embodiments, the ORF has codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level.

In some embodiments, the polypeptide encoded by the ORF is a Cas9 nuclease derived from prokaryotes described below, and an increase in translation in a mammal, cell type, organ of a mammal, human, organ of a human, etc., can be determined relative to the extent of translation wild-type sequence of the ORF, or relative to an ORF of interest, such as an ORF encoding a human protein or transgene for expression in a human cell. For example, the ORF may be an ORF having a codon distribution matching the codon distribution of the organism from which the ORF was derived or the organism that contains the most similar ORF at the amino acid level, such as N. meningitidis, or relative to translation of the Cas9 ORF contained in SEQ ID NO: 29, 32-41, 224-226, 231-233, 238-240, 245-247, 252-254, 259-261, 266-268, 273-275, 280-282, 287-289, 294-296, 301-303, or 316-321 with all else equal, including any applicable point mutations, heterologous domains, and the like. Codons useful for increasing expression in a human, including the human liver and human hepatocytes, can be codons corresponding to highly expressed tRNAs in the human liver/hepatocytes, which are discussed in Dittmar K A, PLOS Genetics 2(12): e221 (2006). In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian liver, such as a human liver. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian hepatocyte, such as a human hepatocyte.

Alternatively, codons corresponding to highly expressed tRNAs in an organism (e.g., human) in general may be used.

Any of the foregoing approaches to codon selection can be combined with selecting codon that contribute to lower repeat content; or using a codon set of Table 1, as shown above; using the minimal uridine or adenine codons shown above, e.g., Table 2, 3, or 4, and then where more than one option is available, using the codon that corresponds to a more highly-expressed tRNA, either in the organism (e.g., human) in general, or in an organ or cell type of interest, such as the liver or hepatocytes (e.g., human liver or human hepatocytes).

### C. Nuclear Localization Signals (NLS)

The nuclear localization signal (NLS) disclosed herein may facilitate transport of the RNA-guided DNA-binding agent into the nucleus of a cell. The first NLS and, when present, the second NLS disclosed herein may be linked at the N-terminus to the RNA-guided DNA-binding agent sequence, i.e., the RNA-guided DNA binding agent is the C-terminal domain in the encoded polypeptide. The first NLS and, when present, the second NLS disclosed herein may be linked at the N-terminus to the NmeCas9 coding sequence. Additional NLS may be linked at the N-terminus of the NmeCas9 coding sequence. In some embodiments, the encoded polypeptide comprises three NLSs at the N-terminus to the NmeCas9 coding sequence. In some embodiments, at least one NLS is provided at the C-terminus of the RNA-guided DNA-binding agent sequence (e.g., with or without an intervening spacer between the NLS and the preceding domain). In some embodiments, a first NLS and a second NLS are provided at the C-terminus of the RNA-guided DNA-binding agent sequence (e.g., with or without an intervening spacer between the NLS and the preceding domain).

Accordingly, in some embodiments, the ORF encoding the polypeptide disclosed herein comprises a coding sequence for the first NLS and a coding sequence for the second NLS such that the encoded first NLS and second NLS are located to N-terminal to the NmeCas9 polypeptide. In some embodiments, the ORF further comprises a coding sequence for a third NLS C-terminal to the ORF encoding the Nine Cas9.

In some embodiments, the NLS may be a monopartite sequence, such as, e.g., the SV40 NLS, PKKKRKV (SEQ ID NO: 388) or PKKKRRV (SEQ ID NO: 421). In some embodiments, the NLS may be a bipartite sequence, such as the NLS of nucleoplasmin, KRPAATKKAGQAKKKK (SEQ ID NO: 422). In some embodiments, the NLS sequence may comprise LAAKRSRTT (SEQ ID NO: 410), QAAKRSRTT (SEQ ID NO: 411), PAPAKRERTT (SEQ ID NO: 412), QAAKRPRTT (SEQ ID NO: 413), RAAKRPRTT (SEQ ID NO: 414), AAAKRSWSMAA (SEQ ID NO: 415), AAAKRVWSMAF (SEQ ID NO: 416), AAAKRSWSMAF (SEQ ID NO: 417), AAAKRKYFAA (SEQ ID NO: 418), RAAKRKAFAA (SEQ ID NO: 419), or RAAKRKYFAV (SEQ ID NO: 420). The NLS may be a snurportin-1 importin-β (IBB domain, e.g., an SPN1-impβ sequence. See Huber et al., 2002, J. Cell Bio., 156, 467-479. In a specific embodiment, a single PKKKRKV (SEQ ID NO: 388). In some embodiments, the first and second NLS are independently selected from an SV40 NLS, a nucleoplasmin NLS, a bipartite NLS, a c-myc like NLS, and an NLS comprising the sequence KTRAD. In certain embodiments, the first and second NLSs may be the same (e.g., two SV40 NLSs). In certain embodiments, the first and second NLSs may be different.

In some embodiments, the first NLS is a SV40NLS and the second NLS is a nucleoplasmin NLS.

In some embodiments, the SV40 NLS comprises a sequence of PKKKRKVE (SEQ ID NO: 383) or KKKRKVE (SEQ ID NO: 384). In some embodiments, the nucleoplasmin NLS comprises a sequence of KRPAATKKAGQAKKKK (SEQ ID NO: 422). In some embodiments, the bipartite NLS comprises a sequence of KRTADGSEFESPKKKRKVE (SEQ ID NO: 385). In some embodiments, the c-myc like NLS comprises a sequence of PAAKKKKLD (SEQ ID NO: 386).

In some embodiments, one or more NLS(s) according to any of the foregoing embodiments are present in the RNA-guided DNA-binding agent in combination with one or more additional heterologous functional domains, such as any of the heterologous functional domains described below.

### D. Other Heterologous Functional Domains

In some embodiments, the polypeptide (e.g., RNA-guided DNA-binding agent) encoded by the ORF described herein comprises one or more additional heterologous functional domains (e.g., is or comprises a fusion polypeptide). In some embodiments, the ORF further comprises a nucleotide sequence encoding one or more additional heterologous functional domains.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, calmodulin, and HiBiT. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In certain embodiments, the DNA modification domain is a methylation domain, such as a demethylation or methyltransferase domain. In certain embodiments, the effector domain is a DNA modification domain, such as a base-editing domain. In particular embodiments, the DNA modification domain is a nucleic acid editing domain that introduces a specific modification into the DNA, such as a deaminase domain, which are further discussed below.

Linkers In some embodiments, the ORF further comprises a nucleotide sequence encoding a linker sequence between the first NLS and the second NLS.

In some embodiments, the ORF further comprises a nucleotide sequence encoding a linker sequence between the Nine Cas9 coding sequence and the NLS proximal to the Nine Cas9 coding sequence.

In some embodiments, the spacer comprises at least 1, 2, 3, 4, 5, 6, 7, 8, 9, 10, 15, 16, 17, 18, 19, 20, 25, 30, 40, 50, or more amino acids. In some embodiments, the spacer comprises at least 1, 2, 3, 4, 5, 6, 7, 8, 9, 10, 11, 12, 13, 14, 15, 16, 17, 18, 19, or 20 amino acids.

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 58), SGSETPGTSESA (SEQ ID NO: 59), or SGSETPGTSESATPEGGSGGS (SEQ ID NO: 60).

In some embodiments, the peptide linker comprises a (GGGGS)ₙ (SEQ ID NO: 62), a (G)ₙ, an (EAAAK)ₙ(SEQ ID NO: 63), a (GGS)ₙ, (SEQ ID NO: 61), or an SGSETPGTSESATPES (SEQ ID NO: 58) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 61-122.

### E. UTRs; Kozak Sequences

In some embodiments, the polynucleotide comprises at least one UTR from Hydroxysteroid 17-Beta Dehydrogenase 4 (HSD17B4 or HSD), e.g., a 5′ UTR from HSD. In some embodiments, the polynucleotide comprises at least one UTR from a globin mRNA, for example, human alpha globin (HBA) mRNA, human beta globin (HBB) mRNA, or Xenopus laevis beta globin (XBG) mRNA. In some embodiments, the polynucleotide comprises a 5′ UTR, 3′ UTR, or 5′ and 3′ UTRs from a globin mRNA, such as HBA, HBB, or XBG. In some embodiments, the polynucleotide comprises a 5′ UTR from bovine growth hormone, cytomegalovirus (CMV), mouse Hba-a1, HSD, an albumin gene, HBA, HBB, or XBG. In some embodiments, the polynucleotide comprises a 3′ UTR from bovine growth hormone, cytomegalovirus, mouse Hba-a1, HSD, an albumin gene, HBA, HBB, or XBG. In some embodiments, the polynucleotide comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the polynucleotide comprises 5′ and 3′ UTRs that are from the same source, e.g., a constitutively expressed mRNA such as actin, albumin, or a globin such as HBA, HBB, or XBG.

In some embodiments, the polynucleotide disclosed herein comprises a 5′ UTR with at least 90% identity to any one of SEQ ID NOs: 391-398. In some embodiments, the polynucleotide disclosed herein comprises a 3′ UTR with at least 90% identity to any one of SEQ ID NOs: 399-406. In some embodiments, any of the foregoing levels of identity is at least 95%, at least 98%, at least 99%, or 100%. In some embodiments, an mRNA disclosed herein comprises a 5′ UTR having the sequence of any one of SEQ ID NOs: 391-398. In some embodiments, the polynucleotide disclosed herein comprises a 3′ UTR having the sequence of any one of SEQ ID NOs: 399-406.

In some embodiments, the polynucleotide does not comprise a 5′ UTR, e.g., there are no additional nucleotides between the 5′ cap and the start codon. In some embodiments, the mRNA comprises a Kozak sequence (described below) between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the mRNA comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase. In some embodiments, the Kozak sequence is rccAUGg with zero mismatches or with up to one or two mismatches to positions in lowercase. In some embodiments, the Kozak sequence is gccRccAUGG (nucleotides 4-13 of SEQ ID NO: 408; SEQ ID NO: 407) with zero mismatches or with up to one, two, or three mismatches to positions in lowercase. In some embodiments, the Kozak sequence is gccAccAUG with zero mismatches or with up to one, two, three, or four mismatches to positions in lowercase. In some embodiments, the Kozak sequence is GCCACCAUG. In some embodiments, the Kozak sequence is gccgccRccAUGG (SEQ ID NO: 408) with zero mismatches or with up to one, two, three, or four mismatches to positions in lowercase.

### 5′ Cap

In some embodiments, the polynucleotide (e.g., mRNA) disclosed herein comprises a 5′ cap, such as a Cap0, Cap1, or Cap2.

A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

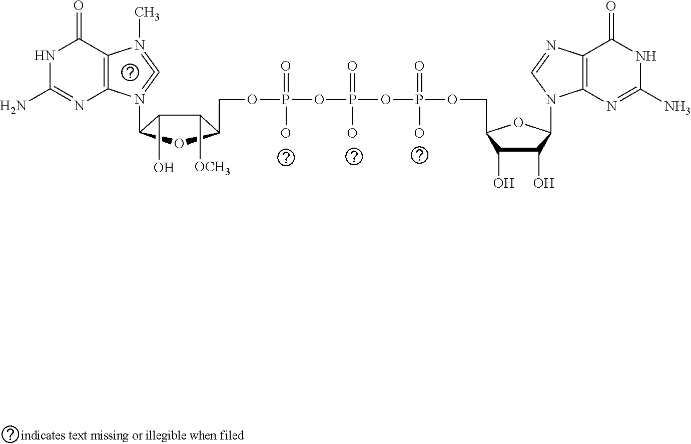

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

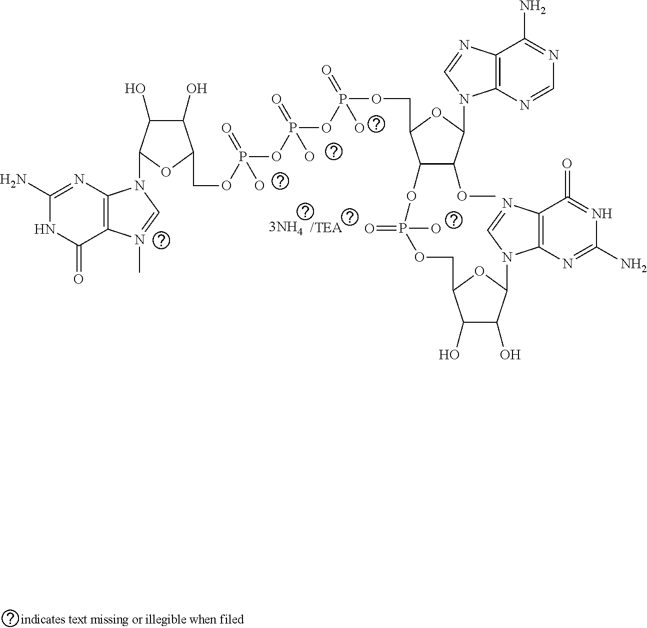

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### F. Poly-A Tail

In some embodiments, the polynucleotide is a mRNA that encodes a polypeptide disclosed herein comprising an ORF, and the mRNA further comprises a poly-adenylated (poly-A) tail.

In some embodiments, the polynucleotide disclosed herein further comprises a poly-A tail sequence or a polyadenylation signal sequence. In some embodiments, the poly-A tail sequence comprises 100-400 nucleotides.

In some embodiments, the poly-A sequence comprises non-adenine nucleotides. In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding a polypeptide disclosed herein. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after at least 8-50 consecutive adenine nucleotides. In some embodiments, the one or more non-adenine nucleotide is located after at least 8-100 consecutive adenine nucleotides. In some embodiments, the non-adenine nucleotide is after one, two, three, four, five, six, or seven adenine nucleotides and is followed by at least 8 consecutive adenine nucleotides.

The poly-A tail of the present disclosure may comprise one sequence of consecutive adenine nucleotides followed by one or more non-adenine nucleotides, optionally followed by additional adenine nucleotides.

In some embodiments, the poly-A tail comprises or contains one non-adenine nucleotide or one consecutive stretch of 2-10 non-adenine nucleotides. In some embodiments, the non-adenine nucleotide(s) is located after at least 8, 9, 10, 11, or 12 consecutive adenine nucleotides. In some instances, the one or more non-adenine nucleotides are located after at least 8-50 consecutive adenine nucleotides. In some embodiments, the one or more non-adenine nucleotides are located after at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive adenine nucleotides.

In some embodiments, the non-adenine nucleotide is guanine, cytosine, or thymine. In some instances, the non-adenine nucleotide is a guanine nucleotide. In some embodiments, the non-adenine nucleotide is a cytosine nucleotide. In some embodiments, the non-adenine nucleotide is a thymine nucleotide. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides. An exemplary poly-A tail comprising non-adenine nucleotides is provided as SEQ ID NO: 409.

In some embodiments, the poly-A tail sequence comprises a sequence of SEQ ID NO: 409.

### G. Modified Nucleotides

In some embodiments, a nucleic acid comprising an ORF encoding a polypeptide disclosed herein comprises a modified uridine at some or all uridine positions.

In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof. In some embodiments the modified uridine is 5-methoxyuridine. In some embodiments the modified uridine is 5-iodouridine. In some embodiments the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in a polynucleotide according to the disclosure are modified uridines. In some embodiments, at least 10% of the uridine of the uridine positions in a polynucleotide according to the disclosure is substituted with a modified uridine. In some embodiments, at least 20% of the uridine of the uridine positions in a polynucleotide according to the disclosure is substituted with a modified uridine. In some embodiments, at least 30% of the uridine of the uridine positions in a polynucleotide according to the disclosure is substituted with a modified uridine. In some embodiments, at least 80% of the uridine of the uridine positions in a polynucleotide according to the disclosure is substituted with a modified uridine. In some embodiments, at least 90% of the uridine of the uridine positions in a polynucleotide according to the disclosure is substituted with a modified uridine.

In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are modified uridine. In some embodiments, 15% to 45% of the uridine of the uridine positions in a polynucleotide according to the disclosure is substituted with a modified uridine.

In some embodiments, 100% of the uridine of the uridine positions in a polynucleotide according to the disclosure is substituted with a modified uridine.

In some embodiments, the modified uridine is one or more of N1-methyl-pseudouridine, pseudouridine, 5-methoxyuridine, or 5-iodouridine, or a combination thereof. In some embodiments, the modified uridine is one or both of N1-methyl-pseudouridine or 5-methoxyuridine. In some embodiments, the modified uridine is N1-methyl-pseudouridine. In some embodiments, the modified uridine is 5-methoxyuridine.

In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-methoxyuridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-iodouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-methoxyuridine, and the remainder are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-iodouridine, and the remainder are N1-methyl pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with the modified uridine, optionally wherein the modified uridine is N1-methyl-pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with N1-methyl-pseudouridine. In some embodiments, 85%, 90%, 95%, or 100% of the uridine positions in a polynucleotide according to the disclosure is substituted with N1-methyl-pseudouridine. In some embodiments, 100% of the uridine is substituted with N1-methyl-pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with the modified uridine, optionally wherein the modified uridine is pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with pseudouridine. In some embodiments, 85%, 90%, 95%, or 100% of the uridine positions in a polynucleotide according to the disclosure is substituted with pseudouridine. In some embodiments, 100% of the uridine is substituted with pseudouridine.

### II. Exemplary Polynucleotides and Compositions Comprising a Deaminase and an RNA-Guided Nickase

The RNA-guided DNA binding agent disclosed herein may further comprise a base-editing domain that introduces a specific modification into a target nucleic acid, such as a deaminase domain.

In some embodiments, a nucleic acid is provided, the nucleic acid comprising an open reading frame encoding a polypeptide comprising a cytidine deaminase (e.g., A3A) and a C-terminal NmeCas9 nickase, and a first nuclear localization signal (NLS), wherein the polypeptide does not comprise a uracil glycosylase inhibitor (UGI).

In some embodiments, a second NLS is N-terminal to the Nine Cas9 nickase. In some embodiments, the deaminase is N-terminal to an NLS (i.e., the first NLS or the second NLS). In some embodiments, the deaminase is N-terminal to all NLS in the polypeptide. In some embodiments, and wherein the polypeptide does not comprise a uracil glycosylase inhibitor (UGI).

In some embodiments, the polynucleotide is DNA or RNA. In some embodiments, the polynucleotide is mRNA. In some embodiments, a polypeptide encoded by the mRNA is provided.

In some embodiments, the polypeptide comprises, from N to C terminus, an optional NLS, a cytidine deaminase (e.g., APOBEC3A), an optional linker, a D16A NmeCas9 nickase. In some embodiments, the polypeptide comprises, from N to C terminus, an optional NLS, a cytidine deaminase (e.g., APOBEC3A), an optional linker, a D16A Nme2Cas9 nickase. In some embodiments, the polypeptide comprises, from N to C terminus, first and second NLSs, a cytidine deaminase (e.g., APOBEC3A), an optional linker, a D16A NmeCas9 nickase. In some embodiments, the polypeptide comprises, from N to C terminus, first and second NLSs, a cytidine deaminase (e.g., APOBEC3A), an optional linker, a D16A Nme2Cas9 nickase. In some embodiments, the polypeptide comprises, from N to C terminus, A first NLS, a cytidine deaminase (e.g., APOBEC3A), a second NLS, an optional linker, a D16A NmeCas9 nickase. In some embodiments, the polypeptide comprises, from N to C terminus, A first NLS, a cytidine deaminase (e.g., APOBEC3A), a second NLS, an optional linker, a D16A Nme2Cas9 nickase.

In some embodiments, the polypeptide comprising A3A and an RNA-guided nickase does not comprise a uracil glycosylase inhibitor (UGI).

In some embodiments, a composition is provided comprising a first polypeptide, or an mRNA encoding a first polypeptide, comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI); and a second polypeptide, or an mRNA encoding a second polypeptide, comprising a uracil glycosylase inhibitor (UGI), wherein the second polypeptide is different from the first polypeptide.

In some embodiments, methods of modifying a target gene are provided comprising administering the compositions described herein. In some embodiments, the method comprises delivering to a cell a first nucleic acid comprising a first open reading frame encoding a first polypeptide comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI), and a second nucleic acid comprising a second open reading frame encoding a uracil glycosylase inhibitor (UGI), wherein the second nucleic acid is different from the first nucleic acid.

In some embodiments, the methods comprise delivering to a cell a polypeptide comprising a deaminase, which is optionally an APOBEC3A deaminase (A3A); a C-terminal NmeCas9 nickase; a first nuclear localization signal (NLS); and a second NLS; wherein the first NLS and the second NLS are located to N-terminal to the sequence encoding the NmeCas9 nickase, wherein the first polypeptide does not comprise a uracil glycosylase inhibitor (UGI), or a nucleic acid encoding the polypeptide, and delivering to the cell a uracil glycosylase inhibitor (UGI), or a nucleic acid encoding the UGI.

In some embodiments, a molar ratio of the mRNA encoding UGI to the mRNA encoding the APOBEC3A deaminase (A3A) and an RNA-guided nickase is from about 1:35 to from about 30:1. In some embodiments, the molar ratio is from about 1:25 to about 25:1. In some embodiments, the molar ratio is from about 1:20 to about 25:1. In some embodiments, the molar ratio is from about 1:10 to about 22:1. In some embodiments, the molar ratio is from about 1:5 to about 25:1. In some embodiments, the molar ratio is from about 1:1 to about 30:1. In some embodiments, the molar ratio is from about 2:1 to about 10:1. In some embodiments, the molar ratio is from about 5:1 to about 20:1. In some embodiments, the molar ratio is from about 1:1 to about 25:1. In some embodiments, the molar ratio may be about 1:35, 1:34, 1:33, 1:32, 1:31, 1:30, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1. In some embodiments, the molar ratio is equal to or larger than about 1:1. In some embodiments the molar ratio is about 1:1. In some embodiments the molar ratio is about 2:1. In some embodiments the molar ratio is about 3:1. In some embodiments the molar ratio is about 4:1. In some embodiments the molar ratio is about 5:1. In some embodiments the molar ratio is about 6:1. In some embodiments the molar ratio is about 7:1. In some embodiments the molar ratio is about 8:1. In some embodiments the molar ratio is about 9:1. In some embodiments the molar ratio is about 10:1. In some embodiments the molar ratio is about 11:1. In some embodiments the molar ratio is about 12:1. In some embodiments the molar ratio is about 13:1. In some embodiments the molar ratio is about 14:1. In some embodiments the molar ratio is about 15:1. In some embodiments the molar ratio is about 16:1. In some embodiments the molar ratio is about 17:1. In some embodiments the molar ratio is about 18:1. In some embodiments the molar ratio is about 19:1. In some embodiments the molar ratio is about 20:1. In some embodiments the molar ratio is about 21:1. In some embodiments the molar ratio is about 22:1. In some embodiments the molar ratio is about 23:1. In some embodiments the molar ratio is about 24:1. In some embodiments the molar ratio is about 25:1.

Similarly, in some embodiments, the molar ratio discussed above for the mRNA encoding the UGI protein to the mRNA encoding the APOBEC3A deaminase (A3A) and an RNA-guided nickase are similar if delivering protein.

In some embodiments, the composition described herein further comprises at least one gRNA. In some embodiments, a composition is provided that comprises an mRNA described herein and at least one gRNA. In some embodiments, the gRNA is a single guide RNA (sgRNA). In some embodiments, the gRNA is a dual guide RNA (dgRNA).

In some embodiments, the composition is capable of effecting genome editing upon administration to the subject.

### A. Cytidine Deaminase; APOBEC3A Deaminase

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); and Carrington et al., Cells 9:1690 (2020)).

In some embodiments, the cytidine deaminase disclosed herein is an enzyme of APOBEC family. In some embodiments, the cytidine deaminase disclosed herein is an enzyme of APOBEC1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A). In some embodiments, the deaminase comprises an APOBEC3A deaminase.

In some embodiments, an APOBEC3A deaminase (A3A) disclosed herein is a human A3A. In some embodiments, an APOBEC3A deaminase (A3A) disclosed herein is a human A3A. In some embodiments, the A3A is a wild-type A3A.

In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, at least about 99% identity, at least about 99.5% identity, or at least about 99.9% identity to a wild type A3A. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A. In some embodiments, the A3A variant comprises a fragment of an A3A, such that the fragment has at least about 80% identity, at least about 90% identity, at least about 95% identity, at least about 96% identity, at least about 97% identity, at least about 98% identity, at least about 99% identity, at least about 99.5% identity, or at least about 99.9% identity to the corresponding fragment of a wild-type A3A.

In some embodiments, an A3A variant is a protein having a sequence that differs from a wild-type A3A protein by one or several mutations, such as substitutions, deletions, insertions, one or several single point substitutions. In some embodiments, a shortened A3A sequence could be used, e.g., by deleting N-terminal, C-terminal, or internal amino acids. In some embodiments, a shortened A3A sequence is used where one to four amino acids at the C-terminus of the sequence is deleted. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

In some embodiments, the wild-type A3A is a human A3A (UniProt accession ID: p319411, SEQ ID NO: 151).

In some embodiments, the A3A disclosed herein comprises an amino acid sequence having at least 80% identity to SEQ ID NO: 151. In some embodiments, the level of identity is at least 85%, at least 87%, at least 90%, at least 95%, at least 98%, at least 99%, or 100%. In some embodiments, the A3A comprises an amino acid sequence having at least 87% identity to SEQ ID NO: 151. In some embodiments, the A3A comprises an amino acid sequence with at least 90% identity to SEQ ID NO: 151. In some embodiments, the A3A comprises an amino acid sequence with at least 95% identity to SEQ ID NO: 151. In some embodiments, the A3A comprises an amino acid sequence with at least 98% identity to SEQ ID NO: 151. In some embodiments, the A3A comprises an amino acid sequence with at least 99% identity to A3A ID NO: 151. In some embodiments, the A3A comprises the amino acid sequence of SEQ ID NO: 151.

In some embodiments, the cytidine deaminase disclosed herein comprises an amino acid sequence having at least 80% identity to any one of SEQ ID NO: 151-216. In some embodiments, the level of identity is at least 85%, at least 87%, at least 90%, at least 95%, at least 98%, at least 99%, or 100%. In some embodiments, the cytidine deaminase comprises the amino acid sequence of any one of SEQ ID NOs: 151-216.

### B. UGI

Without being bound by any theory, providing a UGI together with a polypeptide comprising a deaminase may be helpful in the methods described herein by inhibiting cellular DNA repair machinery (e.g., UDG and downstream repair effectors) that recognize a uracil in DNA as a form of DNA damage or otherwise would excise or modify the uracil or surrounding nucleotides. It should be understood that the use of a UGI may increase the editing efficiency of an enzyme that is capable of deaminating C residues.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264: 1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each are incorporated herein by reference. It should be appreciated that any proteins that are capable of inhibiting a uracil-DNA glycosylase base-excision repair enzyme are within the scope of the present disclosure. Additionally, any proteins that block or inhibit base-excision repair as also within the scope of this disclosure. In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a single-stranded binding protein. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. In some embodiments, a uracil glycosylase inhibitor is a catalytically inactive UDG.

In some embodiments, a uracil glycosylase inhibitor (UGI) disclosed herein comprises an amino acid sequence with at least 80% to SEQ ID NO: 3. In some embodiments, any of the foregoing levels of identity is at least 90%, at least 95%, at least 98%, at least 99%, or 100%. In some embodiments, the UGI comprises an amino acid sequence with at least 90% identity to SEQ ID NO: 3. In some embodiments, the UGI comprises an amino acid sequence with at least 95% identity to SEQ ID NO: 3. In some embodiments, the UGI comprises an amino acid sequence with at least 98% identity to SEQ ID NO: 3. In some embodiments, the UGI comprises an amino acid sequence with at least 99% identity to SEQ ID NO: 3. In some embodiments, the UGI comprises the amino acid sequence of SEQ ID NO: 3.

### C. Linkers

In some embodiments, the polypeptide comprising the deaminase and the RNA-guided nickase described herein further comprises a linker that connects the deaminase and the RNA-guided nickase. In some embodiments, the linker is a peptide linker. In some embodiments, the nucleic acid encoding the polypeptide comprising the deaminase and the RNA-guided nickase further comprises a sequence encoding the peptide linker. In some embodiments, mRNAs encoding the deaminase-linker-RNA-guided nickase fusion protein are provided.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 58), SGSETPGTSESA (SEQ ID NO: 59), or SGSETPGTSESATPEGGSGGS (SEQ ID NO: 60).

In some embodiments, the peptide linker comprises a (GGGGS)ₙ (SEQ ID NO: 62), a (G)ₙ, an (EAAAK)ₙ(SEQ ID NO: 63), a (GGS)ₙ (SEQ ID NO: 61), or an SGSETPGTSESATPES (SEQ ID NO: 58) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 58-122. In some embodiments, the peptide linker comprises one or more sequences selected from SEQ ID NO: 58, SEQ ID NO: 59, SEQ ID NO: 60, SEQ ID NO: 118, SEQ ID NO: 119, SEQ ID NO: 120. SEQ ID NO: 121, and SEQ ID NO: 122.

### D. Compositions Comprising an APOBEC3A Deaminase and an RNA-Guided Nickase

In some embodiments, an mRNA encoding a polypeptide comprising a cytidine deaminase (e.g., A3A) and an RNA-guided nickase is provided. In some embodiments, the polypeptide comprises a human deaminase (e.g., A3A) and a C-terminal RNA-guided nickase; and a nucleotide sequence encoding a first NLS and optionally a second NLS. In certain embodiments, the deaminase is N-terminal to an NLS. In certain embodiments, the deaminase is N-terminal to all NLS.

In some embodiments, the polypeptide comprises a wild-type deaminase (e.g., A3A) and a C-terminal RNA-guided nickase. In some embodiments, the polypeptide comprises an A3A variant and an RNA-guided nickase. In some embodiments, the polypeptide comprises a deaminase (e.g., A3A) and a Cas9 nickase. In some embodiments, the polypeptide comprises a deaminase (e.g., A3A) and a D16A NmeCas9 nickase. In some embodiments, the polypeptide comprises a human deaminase (e.g., A3A) and a D16A NmeCas9 nickase. In some embodiments, the polypeptide comprises an A3A variant and a D16A NmeCas9 nickase. In some embodiments, the polypeptide lacks a UGI. In some embodiments, the deaminase (e.g., A3A) and the RNA-guided nickase are linked via a linker. In some embodiments, the polypeptide further comprises one or more additional heterologous functional domains. In some embodiments, the polypeptide further comprises a nuclear localization sequence (NLS) (described herein).

In some embodiments, the polypeptide comprises a human deaminase (e.g., A3A) and a C-terminal D16A NmeCas9 nickase, wherein the human deaminase (e.g., A3A) and the D16A NmeCas9 are fused via a linker. In some embodiments, the polypeptide comprises a human A3A and a C-terminal D16A NmeCas9 nickase, and a NLS at the N-terminus of the fused polypeptide. In some embodiments, the polypeptide comprises a human A3A and a C-terminal D16A NmeCas9 nickase, wherein the human A3A and the D16A NmeCas9 are fused via a linker, and a NLS fused to the N-terminus of the human A3A, optionally via a linker.

The polypeptide may be organized in any number of ways to form a single chain. The first NLS and, when present, the second NLS are located to N-terminal to the sequence encoding the Cas9 nickase. Additional NLS can be N-terminal to the Cas9 nickase. The A3A can be N- or C-terminal as compared an NLS. In some embodiments, the polypeptide comprises, from N to C terminus, a first NLS, an optional second NLS, a deaminase, an optional linker, an RNA-guided nickase, and an optional NLS. In some embodiments, linkers are independently present between the first and second NLS, and an NLS and a deaminase. In some embodiments, the polypeptide comprises, from N to C terminus, a deaminase, a first NLS, an optional second NLS, a C-terminal RNA-guided nickase. In some embodiments, linkers are independently present between a deaminase and a first NLS, between a first NLS and a second NLS, and between an NLS and a C-terminal nickase.

In any of the foregoing embodiments, the polypeptide may comprise an amino acid sequence having at least 80% identity to SEQ ID NOs: 14. In some embodiments, any of the foregoing levels of identity is at least 85%, 90%, 95%, 98%, or 99%, or 100% identical. In some embodiments, the polypeptide disclosed herein may comprise an amino acid sequence with at least 90% identity to SEQ ID NOs: 14. In some embodiments, the polypeptide disclosed herein may comprise an amino acid sequence with at least 95% identity to SEQ ID NOs: 3 or 6. In some embodiments, the polypeptide disclosed herein may comprise an amino acid sequence with at least 98% identity to SEQ ID NOs: 14. In some embodiments, the polypeptide disclosed herein may comprise an amino acid sequence with at least 99% identity to SEQ ID NOs: 14. In some embodiments, the polypeptide disclosed herein may comprise an amino acid sequence of SEQ ID NOs: 14.

In any of the foregoing embodiments, a nucleic acid sequence comprising an open reading frame encoding the polypeptide disclosed herein may comprise a nucleic acid sequence having at least 80% identity to SEQ ID NOs: 42. In some embodiments, any of the foregoing levels of identity is at least 85%, 90%, 95%, 98%, or 99%, or 100% identical.

In any of the foregoing embodiments, an mRNA sequence encoding the polypeptide disclosed herein may comprise a nucleic acid sequence having at least 80% identity to SEQ ID NOs: 28. In some embodiments, any of the foregoing levels of identity is at least 85%, 90%, 95%, 98%, or 99%, or 100% identical.

In any of the foregoing embodiments, the A3A may comprise an amino acid sequence having at least 80% identity to SEQ ID NO: 151. In some embodiments, the level of identity is at least 85%, 87%, 90%, 95%, 98%, or 99%, or 100% identical. In some embodiments, the A3A comprises an amino acid sequence of SEQ ID NO: 151.

In any of the foregoing embodiments, the NmeCas9 nickase may comprise an amino acid sequence having at least 80%, 90%, 95%, 98%, or 99% identity to any one of SEQ ID NOs: 220, 248, or 276. In some embodiments, the level of identity is at least 85%, 87%, 90%, 95%, 98%, or 99%, or 100% identical. In some embodiments, the RNA-guided nickase comprises the amino acid sequence of SEQ ID NO: 220, 248, or 276. In some embodiments, the RNA-guided nickase comprises the amino acid sequence of SEQ ID NO: 220, 248, or 276. In some embodiments, the RNA-guided nickase comprises the amino acid sequence of SEQ ID NO: 220, 248, or 276.

### III. Guide RNA

In some embodiments, at least one guide RNA is provided in combination with a polynucleotide disclosed herein, such as a polynucleotide encoding an RNA-guided DNA-binding agent. In some embodiments, a guide RNA is provided as a separate molecule from the polynucleotide. In some embodiments, a guide RNA is provided as a part, such as a part of a UTR, of a polynucleotide disclosed herein.

In some embodiments, a composition comprising the polynucleotide disclosed herein further comprises at least one guide RNA (or “gRNA”).

In some embodiments, the gRNA is a single guide RNA (or “sgRNA”).

In some embodiments, the gRNA is a dual guide RNA.

In some embodiments, a guide RNA comprises a modified sgRNA. A sgRNA may be modified to improve its in vivo stability.

In some embodiments, a gRNA described herein is an N. meningitidis Cas9 (NmeCas9) gRNA comprising a conserved portion comprising a repeat/anti-repeat region, a hairpin 1 region, and a hairpin 2 region, wherein one or more of the repeat/anti-repeat region, the hairpin 1 region, and the hairpin 2 region are shortened. Exemplary wild-type NmeCas9 guide RNA comprises a sequence of (N)₂₀₋₂₅ GUUGUAGCUCCCUUUCUCAUUUCGGAAACGAAAUGAGAACCGUUGCUACAAU AAGGCCGUCUGAAAAGAUGUGCCGCAACGCUCUGCCCCUUAAAGCUUCUGCUU UAAGGGGCAUCGUUUA (SEQ ID NO: 500). (N)₂₀₋₂₅ as used herein represent 20-25, i.e., 20, 21, 22, 23, 24, or 25 consecutive N. A, C, G, and U represent nucleotides having adenine, cytosine, guanine, and uracil bases, respectively. In some embodiments, (N)₂₀₋₂₅ has 24 nucleotides in length. N is any natural or non-natural nucleotide, and where the totality of the N's comprises a guide sequence.

In some embodiments, the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500; and wherein at least 10 nucleotides are modified nucleotides.

In some embodiments, the shortened repeat/anti-repeat region of the gRNA lacks 18 nucleotides. In some embodiments, the shortened repeat/anti-repeat region of the gRNA lacks 22 nucleotides.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotide 36 is linked to nucleotide 65 by 7 nucleotides. In some embodiments, in the shortened repeat/anti-repeat region of the gRNA, nucleotide 36 is linked to nucleotide 65 by 8 nucleotides. In some embodiments, in the shortened repeat/anti-repeat region of the gRNA, nucleotide 36 is linked to nucleotide 65 by 9 nucleotides. In some embodiments, in the shortened repeat/anti-repeat region of the gRNA, nucleotide 36 is linked to nucleotide 65 by 10 nucleotides.

In some embodiments, in the shortened repeat/anti-repeat region of the gRNA, nucleotides 38-48 and 53-63 are deleted relative to SEQ ID NO: 500. In some embodiments, in the shortened repeat/anti-repeat region of the gRNA, nucleotides 38, 41-48, 53-60, and 63 are deleted relative to SEQ ID NO: 500.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotides 38-48 and 53-63 are deleted relative to SEQ ID NO: 500, and nucleotide 36 is linked to nucleotide 65 by nucleotides 37, 49-52, and 64.

In some embodiments, in the shortened repeat/anti-repeat region of the gRNA, nucleotide 36 is linked to nucleotide 65 by 10 nucleotides. In some embodiments, in the shortened repeat/anti-repeat region of the gRNA, nucleotides 38, 41-48, 53-60, and 63 are deleted relative to SEQ ID NO: 500, and nucleotide 36 is linked to nucleotide 65 by nucleotides 37, 39, 40, 49-52, 61, 62, and 64.

In some embodiments, all of nucleotides 38-48 and nucleotides 53-63 of the upper stem of the shortened repeat/anti-repeat region are deleted relative to SEQ ID NO: 500.

In some embodiments, all of nucleotides 39-48 and nucleotides 53-62 of the upper stem of the shortened repeat/anti-repeat region are deleted relative to SEQ ID NO: 500, and nucleotides 38 and 63 is substituted.

In some embodiments, the shortened repeat/anti-repeat region has 14 modified nucleotides. In some embodiments, the shortened repeat/anti-repeat region has 15 modified nucleotides. In some embodiments, the shortened repeat/anti-repeat region has 16 modified nucleotides. In some embodiments, the shortened repeat/anti-repeat region has 17 modified nucleotides. In some embodiments, the shortened repeat/anti-repeat region has 18 modified nucleotides. In some embodiments, the shortened repeat/anti-repeat region has 19 modified nucleotides. In some embodiments, the shortened repeat/anti-repeat region has 20 modified nucleotides.

In some embodiments, the shortened hairpin 1 region lacks 2 nucleotides. In some embodiments, the shortened hairpin 1 region lacks 21 nucleotides. In some embodiments, the shortened hairpin 1 region lacks 2 nucleotides, and nucleotides 86 and 91 are deleted relative to SEQ ID NO: 500. In some embodiments, the shortened hairpin 1 region lacks 2 nucleotides, and nucleotides 85 and 92 are deleted relative to SEQ ID NO: 500. In some embodiments, in the shortened hairpin 1 region, nucleotide 81 is linked to nucleotide 96 by 12 nucleotides. In some embodiments, in the shortened hairpin 1 region, nucleotide 81 is linked to nucleotide 96 by 12 nucleotides. In some embodiments, in the shortened hairpin 1 region, nucleotides 86 and 91 are deleted relative to SEQ ID NO: 500, and nucleotide 81 is linked to nucleotide 96 by nucleotides 82-85, 87-90, and 92-95. In some embodiments, in the shortened hairpin 1 region, nucleotides 85 and 92 are deleted relative to SEQ ID NO: 500, and nucleotide 81 is linked to nucleotide 96 by nucleotides 82-84, 86-91, and 93-95.

In some embodiments, the shortened hairpin 1 region has a duplex portion of 7 base paired nucleotides in length. In some embodiments, the shortened hairpin 1 region has a duplex portion of 8 base paired nucleotides in length.

In the stem of the shortened hairpin 1 region is seven base paired nucleotides in length. In some embodiments, nucleotides 85-86 and nucleotides 91-92 of the shortened hairpin 1 region are deleted.

In some embodiments, the shortened hairpin 1 region has 13 modified nucleotides.

In some embodiments, the shortened hairpin 2 lacks 18 nucleotides. In some embodiments, the shortened hairpin 2 has 24 nucleotides. In some embodiments, in the shortened hairpin 2 nucleotides 113-121 and 126-134 are deleted relative to SEQ ID NO: 500. In some embodiments, the shortened hairpin 2 lacks 18 nucleotides, and nucleotides 113-121 and 126-134 are deleted relative to SEQ ID NO: 500. In some embodiments, in the shortened hairpin 2 region, nucleotide 112 is linked to nucleotide 135 by 4 nucleotides. In some embodiments, in the shortened hairpin 2 region, nucleotides 113-121 and 126-134 are deleted relative to SEQ ID NO: 500 and nucleotide 112 is linked to nucleotide 135 by nucleotides 122-125.

In some embodiments, the shortened repeat/anti-repeat region has a length of 28 nucleotides. In some embodiments, the shortened repeat/anti-repeat region has a length of 32 nucleotides.

In some embodiments, the upper stem of the shortened repeat/anti-repeat region comprises no more than one base pair. In some embodiments, the upper stem of the shortened repeat/anti-repeat region comprises no more than three base pairs.

In some embodiments, the shortened hairpin 2 region has 8 modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 500, wherein
   (i) nucleotides 38-48 and 53-63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500; and
(c) a shortened hairpin 2 region, wherein the shortened hairpin 2 lacks 18 nucleotides, wherein nucleotides 113-121 and 126-134 are deleted relative to SEQ ID NO: 500; and wherein nucleotides 144-145 are deleted relative to SEQ ID NO: 500; wherein at least 10 nucleotides are modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 500, wherein
   (i) nucleotides 38, 41-48, 53-60, and 63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500;
(c) a shortened hairpin 2 region, wherein the shortened hairpin 2 lacks 18 nucleotides, wherein nucleotides 113-121 and 126-134 are deleted relative to SEQ ID NO: 500; and
wherein nucleotides 144-145 are deleted relative to SEQ ID NO: 500;
wherein at least 10 nucleotides are modified nucleotides.

In some embodiments, a guide RNA (gRNA) is provided, the gRNA comprising a guide region and a conserved region, the conserved region comprising one or more of:
(a) a shortened repeat/anti-repeat region, wherein the shortened repeat/anti-repeat region lacks 18-22 nucleotides relative to SEQ ID NO: 500, wherein
   (i) nucleotides 37-48 and 53-64 are deleted; and
   (ii) nucleotide 36 is linked to nucleotide 65 by 6-10 nucleotides; or
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 500; or
(c) a shortened hairpin 2 region, wherein the shortened hairpin 2 lacks 18 nucleotides, wherein nucleotides 113-121 and 126-134 are deleted relative to SEQ ID NO: 500; and
wherein nucleotides 144-145 are deleted relative to SEQ ID NO: 500;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 500. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC.

In some embodiments, the guide RNA (gRNA) of the previous embodiment comprising a guide region and a conserved region, the conserved region comprising:
(a) a shortened repeat/anti-repeat region, wherein the shortened repeat/anti-repeat region lacks 18-22 nucleotides, wherein
   (i) nucleotides 37-48 and 53-64 are deleted relative to SEQ ID NO: 500; and
   (ii) nucleotide 36 is linked to nucleotide 65 by 6-10 nucleotides;
(b) a shortened hairpin 1 region, wherein the shortened hairpin 1 lacks 2 nucleotides relative to SEQ ID NO: 500, wherein nucleotides 86 and 91 are deleted or nucleotides 85 and 92 are deleted;
(c) a shortened hairpin 2 region, wherein the shortened hairpin 2 lacks 18 nucleotides, wherein nucleotides 113-121 and 126-134 are deleted relative to SEQ ID NO: 500; and
(d) wherein nucleotides 144-145 are deleted relative to SEQ ID NO: 500;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 500. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC.

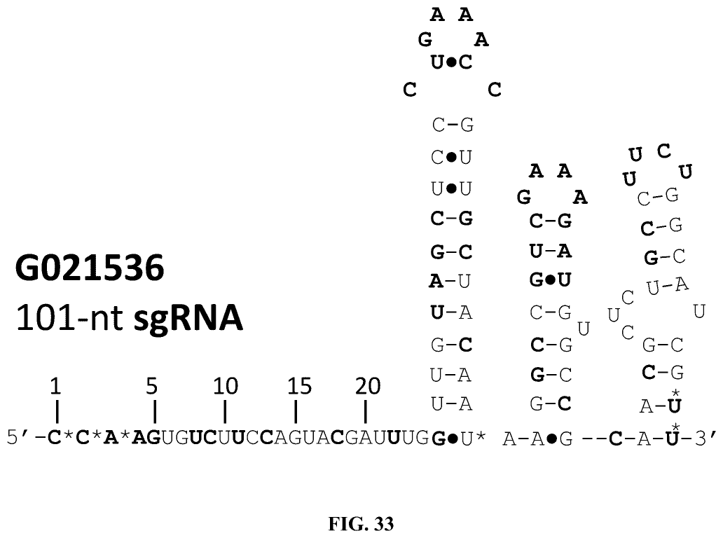

FIGS. 33-35 show exemplary sgRNAs in possible secondary structures.

In some embodiments, the NmeCas9 short-sgRNA comprises one of the following sequences in 5′ to 3′ orientation:

(SEQ ID NO: 501)
(N)₂₀₋₂₅
GUUGUAGCUCCCUGAAACCGUUGCUACAAUAAGGCCGUCGAAAGAUGU
GCCGCAACGCUCUGCCUUCUGGCAUCGUU;
(SEQ ID NO: 502)
(N)₂₀₋₂₅
GUUGUAGCUCCCUGAAACCGUUGCUACAAUAAGGCCGUCGAAAGAUGU
GCCGCAACGCUCUGCCUUCUGGCAUCGUUUAUU;
(SEQ ID NO: 503)
(N)₂₀₋₂₅
GUUGUAGCUCCCUGGAAACCCGUUGCUACAAUAAGGCCGUCGAAAGA
UGUGCCGCAACGCUCUGCCUUCUGGCAUCGUUUAUU;
or
(SEQ ID NO: 514)
(N)₂₀₋₂₅
GUUGUAGCUCCCUUCGAAAGACCGUUGCUACAAUAAGGCCGUCGAAAGAU
GUGCCGCAACGCUCUGCCUUCUGGCAUCGUU,

where N are nucleotides encoding a guide sequence. In some embodiments, N equals 24. In some embodiments, N equals 25. N represents a nucleotide having any base, e.g., A, C, G, or U. (N)₂₀₋₂₅ represent 20-25, i.e., 20, 21, 22, 23, 24, or 25 consecutive N.

In some embodiments, at least 10 nucleotides of the conserved portion of the NmeCas9 short-sgRNA are modified nucleotides.

In some embodiments, the NmeCas9 short-sgRNA comprises a conserved region comprising one of the following sequences in 5′ to 3′ orientation: mGUUGmUmAmGmCUCCCmUmGmAmAmAmCmCGUUmGmCUAmCAAU*AAGmG mCCmGmUmCmGmAmAmAmGmAmUGUGCmCGCmAmAmCmGCUCUmGmCCmUm UmCmUGmGCmAmUC*mG*mU*mU (SEQ ID NO: 504); mGUUGmUmAmGmCUCCCmUmGmAmAmAmCmCGUUmGmCUAmCAAU*AAGmG mCCmGmUmCmGmAmAmAmGmAmUGUGCmCGmCAAmCGCUCUmGmCCmUmUm CmUGGCAUCG*mU*mU (SEQ ID NO: 505); or mGUUGmUmAmGmCUCCCmUmUmCmGmAmAmAmGmAmCmCGUUmGmCUAmCA AU*AAGmGmCCmGmUmCmGmAmAmAmGmAmUGUGCmCGmCAAmCGCUCUmG mCCmUmUmCmUGGCAUCG*mU*mU (SEQ ID NO: 515). Additional examples of the NmeCas9 short-gRNA (e.g., SEQ ID NOs: 512-530) are provided in Table 39B.

In some embodiments, the NmeCas9 short-gRNA comprises one of the following sequences in 5′ to 3′ orientation:

(SEQ ID NO: 512)
mN*mN*mN*mNmNNNmNmNNmNNmNNNNNmNNNNmNNNmGUUGmUmAmGmC
UCCCmUmGmAmAmAmCmCGUUmGmCUAmCAAU*AAGmGmCCmGmUmCmGmA
mAmAmGmAmUGUGCmCGmCAAmCGCUCUmGmCCmUmUmCmUGGCAUCG*mU
*mU;
(SEQ ID NO: 525)
mN*mN*mN*mNmNNNmNmNNmNNmNNNNNmNNNNmNNNmGUUGmUmAmGmC
UCCCmUmGmAmAmAmCmCGUUmGmCUAmCAAUAAGmGmCCmGmUmCmGmAm
AmAmGmAmUGUGCmCGmCAAmCGCUCUmGmCCmUmUmCmUGGCAUCG*mU*
mU;
(SEQ ID NO: 526)
mN*mN*mN*mNmNNNmNmNNmNNmNNNNNmNNNNmNNNmGUUGmUmAmGmC
UCCCmUmGmAmAmAmCmCGUUmGmCUAmCAAU*AAGmGmCCmGmUmCmGmA
mAmAmGmAmUGUGCmCGmCAAmCGmCmUmCmUmGmCCmUmUmCmUGGCAUC
G*mU*mU;
(SEQ ID NO: 527)
mN*mN*mN*mNmNNNmNmNNmNNmNNNNNmNNNNmNNNmGUUGmUmAmGmC
UCCCmUmGmAmAmAmCmCGUUmGmCUAmCAAUAAGmGmCCmGmUmCmGmAm
AmAmGmAmUGUGCmCGmCAAmCGmCmUmCmUmGmCCmUmUmCmUGGCAUCG
*mU*mU;
(SEQ ID NO: 516)
mN*mN*mN*mNmNNNmNmNNmNNmNNNNNmNNNNmNNNmGUUGmUmAmGmC
UCCCmUmUmCmGmAmAmAmGmAmCmCGUUmGmCUAmCAAU*AAGmGmCCmG
mUmCmGmAmAmAmGmAmUGUGCmCGmCAAmCGCUCUmGmCCmUmUmCmUGG
CAUCG*mU*mU;
(SEQ ID NO: 520)
mN*mN*mN*mNmNNNmNmNNmNNmNNNNNmNNNNmNNNmGUUGmUmAmGmC
UCCCmUmUmCmGmAmAmAmGmAmCmCGUUmGmCUAmCAAUAAGmGmCCmGm
UmCmGmAmAmAmGmAmUGUGCmCGmCAAmCGCUCUmGmCCmUmUmCmUGGC
AUCG*mU*mU;
(SEQ ID NO: 521)
mN*mN*mN*mNmNNNmNmNNmNNmNNNNNmNNNNmNNNmGUUGmUmAmGmC
UCCCmUmUmCmGmAmAmAmGmAmCmCGUUmGmCUAmCAAU*AAGmGmCCmG
mUmCmGmAmAmAmGmAmUGUGCmCGmCAAmCGmCmUmCmUmGmCCmUmUmC
mUGGCAUCG*mU*mU;
or
(SEQ ID NO: 522)
mN*mN*mN*mNmNNNmNmNNmNNmNNNNNmNNNNmNNNmGUUGmUmAmGmC
UCCCmUmUmCmGmAmAmAmGmAmCmCGUUmGmCUAmCAAUAAGmGmCCmGm
UmCmGmAmAmAmGmAmUGUGCmCGmCAAmCGmCmUmCmUmGmCCmUmUmCm
UGGCAUCG*mU*mU;
(SEQ ID NO: 523)
(N)₂₀₋₂₅
mGUUGmUmAmGmCUCCCmUmGmAmAmAmCmCGUUmGmCUAmCAAUAAGmGm
CCmGmUmCmGmAmAmAmGmAmUGUGCmCGCmAmAmCmGmCmUmCmUmGmCC
mUmUmCmUGmGCmAmUC*mG*mU*mU;
or
(SEQ ID NO: 524)
(N)₂₀₋₂₅
mGUUGmUmAmGmCUCCCmUmUmCmGmAmAmAmGmAmCmCGUUmGmCUAmCA
AUAAGmGmCCmGmUmCmGmAmAmAmGmAmUGUGCmCGmCAAmCGmCmUmCm
UmGmCCmUmUmCmUGGCAUCG*mU*mU,

wherein N represents a nucleotide having any base, e.g., A, C, G, or U. (mN*)₃ represents three consecutive nucleotides each having any base, a 2′-OMe, and a 3′ PS linkage to the next nucleotide, respectively. Nucleotide modifications are indicated as m is 2′-OMe modification and * is a PS linkage. In the context of a modified nucleotide sequence, in certain embodiments, N, A, C, G, and U are unmodified RNA nucleotides, i.e., 2′-OH and phosphodiesterase linkage to the 3′ nucleotide.

The shortened NmeCas9 gRNA may comprise internal linkers disclosed herein.

“Internal linker” as used herein describes a non-nucleotide segment joining two nucleotides within a guide RNA. If the gRNA contains a spacer region, the internal linker is located outside of the spacer region (e.g., in the scaffold or conserved region of the gRNA). For Type V guides, it is understood that the last hairpin is the only hairpin in the structure, i.e., the repeat-anti-repeat region. In some embodiments, the internal linker comprises a PEG-linker disclosed herein. In some embodiments, the internal linker comprises a PEG-linker disclosed herein.

In some embodiments, the single guide RNA comprises a guide region and a conserved region, wherein the conserved region comprises one or more of:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.

Exemplary locations of the linkers are as shown in the following: (N)₂₀₋₂₅GUUGUAGCUCCCUUC(L1)GACCGUUGCUACAAUAAGGCCGUC(L1)GAUGU GCCGCAACGCUCUGCC(L1)GGCAUCGUU (SEQ ID NO: 506). As used herein, (L1) refers to an internal linker having a bridging length of about 15-21 atoms.

In some embodiments, the shortened NmeCas9 guide RNA comprising internal linkers may be chemically modified. Exemplary modifications include a modification pattern of the following sequence:

(SEQ ID NO: 507)
mN*mN*mN*mNmNNNmNmNNmNNmNNNNNmNNNNmNNNmGUUGmUmAmGmC
UCCCmUmUmC(L1)mGmAmCmCGUUmGmCUAmCAAU*AAGmGmCCmGmUmC
(L1)mGmAmUGUGCmCGmCAAmCGCUCUmGmCC(L1)GGCAUCG*mU*mU.

In some embodiments, the sgRNA comprises the modification pattern shown in SEQ ID NOs: 141 and 143-150 (Nine PEG guides), where N is any natural or non-natural nucleotide, and where the totality of the N's comprises a guide sequence.

### IV. Delivery

In some embodiments, a polynucleotide or a composition disclosed herein is formulated in or administered via a lipid nanoparticle; see, e.g., WO2017173054, the contents of which are hereby incorporated by reference in their entirety.

### Lipids

Disclosed herein are various embodiments using lipid nucleic acid assembly compositions comprising nucleic acids(s), or composition(s) described herein. In some embodiments, the lipid nucleic acid assembly composition comprises a nucleic acid (e.g., mRNA) comprising an open reading frame encoding polynucleotide comprising an open reading frame (ORF), the ORF comprising a nucleotide sequence encoding a C-terminal N. meningitidis (Nine) Cas9 polypeptide disclosed herein and a nucleotide sequence encoding a first nuclear localization signal (NLS). In some embodiments, the NmeCas9 is an Nme2Cas9, an Nme1Cas9, or Nme3Cas9.

As used herein, a “lipid nucleic acid assembly composition” refers to lipid-based delivery compositions, including lipid nanoparticles (LNPs) and lipoplexes. LNP refers to lipid nanoparticles<100 nM. LNPs are formed by precise mixing a lipid component (e.g. in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethyl ether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo therapy. In some embodiments, the aqueous solution comprises an RNA, such as an mRNA or a gRNA. In some embodiments, the aqueous solution comprises an mRNA encoding an RNA-guided DNA binding agent, such as Cas9.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides to subjects may be utilized with the guide RNAs and the nucleic acid encoding a NmeCas9 and an NLS described herein.

In some embodiments, the aqueous solution comprises a nucleic acid encoding a polypeptide comprising an A3A and an RNA-guided nickase. A pharmaceutical formulation comprising the lipid nucleic acid assembly composition may optionally comprise a pharmaceutically acceptable buffer.

In some embodiments, the lipid nucleic acid assembly compositions include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### 1. Amine Lipids

In some embodiments, lipid nucleic acid assembly compositions comprise an “amine lipid”, which is, for example an ionizable lipid such as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

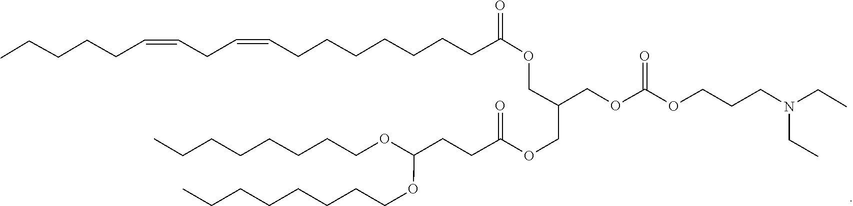 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is an equivalent to Lipid A.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g., an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876, WO/2020/118041, WO/2020/072605, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week-old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., to tumors. See, e.g., WO2014/136086.

### 2. Additional Lipids

“Neutral lipids” suitable for use in a lipid nucleic acid assembly composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine, e.g., 1,2-distearoyl-sn-glycero-3-phosphocholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid nucleic acid assembly composition of the disclosure include, but are not limited to, stealth lipids having a hydrophilic head group linked to a lipid moiety. Stealth lipids suitable for use in a lipid nucleic acid assembly composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (I), wherein n is 45, meaning that the number averaged degree of polymerization comprises about 45 subunits

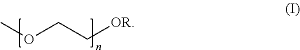

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (e.g., 1,2-dimyristoyl-rac-glycero-3-methylpolyoxyethylene glycol 2000 (PEG2k-DMG) or PEG-DMG (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be 1,2-dimyristoyl-rac-glycero-3-methylpolyoxyethylene glycol 2000 (PEG2k-DMG). In one embodiment, the PEG lipid may be PEG2k-DMG. In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In preferred embodiments, the PEG lipid includes a glycerol group. In preferred embodiments, the PEG lipid includes a dimyristoylglycerol (DMG) group. In preferred embodiments, the PEG lipid comprises PEG-2k. In preferred embodiments, the PEG lipid is a PEG-DMG. In preferred embodiments, the PEG lipid is a PEG-2k-DMG. In preferred embodiments, the PEG lipid is 1,2-dimyristoyl-rac-glycero-3-methoxypolyethylene glycol2000. In preferred embodiments, the PEG-2k-DMG is 1,2-dimyristoyl-rac-glycero-3-methoxypolyethylene glycol-2000.

### LNP

Lipid nanoparticles (LNPs) are a well-known means for delivery of nucleotide and protein cargo, and may be used for delivery of the polynucleotide, compositions, or pharmaceutical formulations disclosed herein. In some embodiments, the LNPs deliver nucleic acid, protein, or nucleic acid together with protein.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical and, in more particular embodiments, can comprise an aqueous core, e.g., comprising a substantial portion of RNA molecules), a dispersed phase in an emulsion, micelles, or an internal phase in a suspension (see, e.g., WO2017173054, the contents of which are hereby incorporated by reference in their entirety). Any LNP known to those of skill in the art to be capable of delivering nucleotides to subjects may be utilized.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), referred to herein as Lipid A. In some embodiments, the LNPs comprise molar ratios of a cationic lipid amine to RNA phosphate (N:P) of about 4.5. In some embodiments, the LNPs comprise is nonyl 8-((7,7-bis(octyloxy)heptyl)(2-hydroxyethyl)amino)octanoate. In some embodiments, the LNPs comprise molar ratios of a cationic lipid amine to RNA phosphate (N:P) of about 4.5-6.5. In some embodiments, the LNPs comprise molar ratios of a cationic lipid amine to RNA phosphate (N:P) of about 4.5. In some embodiments, the LNPs comprise molar ratios of a cationic lipid amine to RNA phosphate (N:P) of about 6.0.

In some embodiments, the present disclosure comprises a method for delivering a polynucleotide or a composition disclosed herein to a subject, wherein the polynucleotide is associated with an LNP. In some embodiments, the present disclosure comprises a method for delivering a first polynucleotide and a second polynucleotide, or a composition for delivering a first polynucleotide and a second polynucleotide to a subject, wherein the first polynucleotide and the second polynucleotide are associated with the same LNP, e.g., co-formulated with the same LNP. In In some embodiments, the present disclosure comprises a method for delivering a first polynucleotide and a second polynucleotide, or a composition for delivering a first polynucleotide and a second polynucleotide to a subject, wherein the first polynucleotide and the second polynucleotide are each associated with a separate LNP, e.g., each polynucleotide is associated with a separate LNP for administration to a subject or use together, e.g., for co-administration. In some embodiments, the first polynucleotide and the second polynucleotide encode an NmeCas9 nickase and a UGI In some embodiments, the composition further comprises one or more guide RNA. In some embodiments, the method further comprises delivering one or more guide RNA.

In some embodiments, provided herein is a method for delivering any of the polynucleotide or composition described herein to a cell or a population of cells or a subject, including to a cell or population of cells in a subject in vivo, wherein any one or more of the components is associated with an LNP. In some embodiments, the composition further comprises one or more guide RNAs. In some embodiments, the method further comprises delivering one or more guide RNAs.

In some embodiments, provided herein is a composition comprising any of the polynucleotide or composition described herein or donor construct disclosed herein, alone or in combination, with an LNP. In some embodiments, the composition further comprises one or more guide RNAs. In some embodiments, the method further comprises delivering one or more guide RNAs.

In some embodiments, LNPs associated with the polynucleotide or composition disclosed herein are for use in preparing a medicament for treating a disease or disorder.

In some embodiments, a method of modifying a target gene is provided, the method comprising delivering to the cell one or more lipid nucleic acid assembly compositions, optionally lipid nanoparticles, comprising the polynucleotide disclosed herein, and one or more guide RNAs.

In some embodiments, at least one lipid nucleic acid assembly composition comprises lipid nanoparticle (LNPs), optionally wherein all lipid nucleic acid assembly compositions comprise LNPs. In some embodiments, at least one lipid nucleic acid assembly composition is a lipoplex composition. In some embodiments, the lipid nucleic acid assembly composition comprises an ionizable lipid.

Electroporation is a well-known means for delivery of cargo, and any electroporation methodology may be used for delivery of a polynucleotide or composition disclosed herein. In some embodiments, electroporation may be used to deliver any one of the a polynucleotide or a composition disclosed herein.

In some embodiments, the present disclosure comprises a method for delivering a polynucleotide, polypeptide, or a composition disclosed herein to an ex vivo cell, wherein the polynucleotide or composition is associated with an LNP or not associated with an LNP. In some embodiments, the LNP is also associated with one or more guide RNAs. See, e.g., PCT/US2021/029446, incorporated herein by reference

In some embodiments, a kit comprising a polynucleotide, a polypeptide, or a composition disclosed herein is provided.

In some embodiments, a pharmaceutical formulation comprising a polynucleotide, polypeptide, or a composition disclosed herein is provided. A pharmaceutical formulation can further comprise a pharmaceutically acceptable carrier, e.g., water or a buffer. A pharmaceutical formulation can further comprise one or more pharmaceutically acceptable excipients, such as a stabilizer, preservative, bulking agent, or the like. A pharmaceutical formulation can further comprise one or more pharmaceutically acceptable salts, such as sodium chloride. In some embodiments, the pharmaceutical formulation is formulated for intravenous administration. In some embodiments, pharmaceutical formulations are non-pyrogenic. In some embodiments, pharmaceutical formulations are sterile, especially for pharmaceutical formulations that are for injection or infusion.

### V. Exemplary Uses, Methods, And Treatments

In some embodiments, a polynucleotide, expression construct, composition, lipid nanoparticle (LNP), or pharmaceutical composition disclosed herein is for use in gene therapy, e.g., of a target gene.

In some embodiments, use of the polynucleotide, composition, or polypeptide of disclosed herein in modifying a target gene in a cell is provided.

In some embodiments, use of the polynucleotide, composition, or polypeptide of disclosed herein in the manufacture of a medicament for modifying a target gene in a cell is provided.

In some embodiments, the polynucleotide or composition is formulated as a lipid nucleic acid assembly composition, optionally a lipid nanoparticle.

In some embodiments, a method of modifying a target gene is provided the method comprising delivering to a cell the polynucleotide, polypeptide, or composition disclosed herein.

In some embodiments, a polynucleotide, expression construct, composition, lipid nanoparticle (LNP), or pharmaceutical composition is for use in genome editing, e.g., editing a target gene wherein the polynucleotide encodes an RNA-guided DNA binding agent (e.g., NmeCas9).

In some embodiments, a polynucleotide, expression construct, composition, lipid nanoparticle (LNP), or pharmaceutical composition disclosed herein encoding a polypeptide disclosed herein is for use in expressing the polypeptide in a heterologous cell, e.g., a human cell or a mouse cell.

In some embodiments, a polynucleotide, expression construct, composition, lipid nanoparticle (LNP), or pharmaceutical composition is for use in modifying a target gene, e.g., altering its sequence or epigenetic status wherein the polynucleotide encodes an RNA-guided DNA binding agent (e.g., NmeCas9).

In some embodiments, a polynucleotide, expression construct, composition, lipid nanoparticle (LNP), or pharmaceutical composition is for use in inducing a double-stranded break (DSB) within a target gene. In some embodiments, a polynucleotide, expression construct, composition, lipid nanoparticle (LNP), or pharmaceutical composition is for use in inducing an indel within a target gene. In some embodiments, the use of a polynucleotide, expression construct, composition, lipid nanoparticle (LNP), or pharmaceutical composition disclosed herein is provided for the preparation of a medicament for genome editing, e.g., editing a target gene wherein the polynucleotide encodes an RNA-guided DNA binding agent (e.g., NmeCas9). In some embodiments, the use of a polynucleotide, expression construct, composition, lipid nanoparticle (LNP), or pharmaceutical composition disclosed herein encoding a polypeptide disclosed herein is provided for the preparation of a medicament for expressing the polypeptide in a heterologous cell or increasing the expression of the polypeptide, e.g., a human cell or a mouse cell. In some embodiments, the use of a polynucleotide, expression construct, composition, lipid nanoparticle (LNP), or pharmaceutical composition disclosed herein is provided for the preparation of a medicament for modifying a target gene, e.g., altering its sequence or epigenetic status. In some embodiments, the use of a polynucleotide, expression construct, composition, lipid nanoparticle (LNP), or pharmaceutical composition disclosed herein is provided for the preparation of a medicament for inducing a double-stranded break (DSB) within a target gene. In some embodiments, the use of a polynucleotide, expression construct, composition, lipid nanoparticle (LNP), or pharmaceutical composition disclosed herein is provided for the preparation of a medicament for inducing an indel within a target gene.

In some embodiments, the target gene is a transgene. In some embodiments, the target gene is an endogenous gene. The target gene may be in a subject, such as a mammal, such as a human. In some embodiments, the target gene is in an organ, such as a liver, such as a mammalian liver, such as a human liver. In some embodiments, the target gene is in a liver cell, such as a mammalian liver cell, such as a human liver cell. In some embodiments, the target gene is in a hepatocyte, such as a mammalian hepatocyte, such as a human hepatocyte. In some embodiments, the liver cell or hepatocyte is in situ. In some embodiments, the liver cell or hepatocyte is isolated, e.g., in a culture, such as in a primary culture. In some embodiments, the target cell is a peripheral blood mononuclear cell (PBMC), such as a mammalian PBMC, such as a human PBMC. In some embodiments, the PBMC is an immune cell, e.g., a T cell, a B cell, an NK cell. In some embodiments, the cell is a pluripotent cell, such as a mammalian pluripotent cell, such as a human pluripotent cell. In some embodiments, the target cell is a stem cell, such as a mammalian stem cell, such as a human stem cell. In some embodiments, the stem cell is present in bone marrow. In some embodiments, the stem cell is an induced pluripotent stem cell (iPCS). In some embodiments, the cells are isolated, e.g., in culture ex vivo.

Also provided are methods corresponding to the uses disclosed herein, which comprise administering the polynucleotide, expression construct, composition, lipid nanoparticle (LNP), or pharmaceutical composition disclosed herein to a subject or contacting a cell such as those described above with the polynucleotide, LNP, or pharmaceutical composition disclosed herein, e.g., to express a polypeptide disclosed herein or increase the expression of a polypeptide disclosed herein, e.g., in a heterologous cell, such as a human cell or a mouse cell.

In any of the foregoing embodiments involving a subject, the subject can be a mammal. In any of the foregoing embodiments involving a subject, the subject can be human.

In some embodiments, a polynucleotide, expression construct, composition, lipid nanoparticle (LNP), or pharmaceutical composition disclosed herein is administered intravenously or for intravenous administration.

In some embodiments, a single administration of a polynucleotide, LNP, or pharmaceutical composition disclosed herein is sufficient to knock down expression of the target gene product. In some embodiments, a single administration of a polynucleotide, LNP, or pharmaceutical composition disclosed herein is sufficient to knock out expression of the target gene product. In other embodiments, more than one administration of a polynucleotide, LNP, or pharmaceutical composition disclosed herein may be beneficial to maximize editing, modification, indel formation, DSB formation, or the like via cumulative effects.

### VI. Exemplary DNA Molecules, Vectors, Expression Constructs, Host Cells, and Production Methods

In certain embodiments, the present disclosure provides a DNA molecule comprising an ORF sequence encoding a polypeptide disclosed herein. In some embodiments, in addition to the ORF sequence, the DNA molecule further comprises nucleic acids that do not encode the polypeptide disclosed herein. Nucleic acids that do not encode the polypeptide include, but are not limited to, promoters, enhancers, regulatory sequences, and nucleic acids encoding a guide RNA.

In some embodiments, the DNA molecule further comprises a nucleotide sequence encoding a crRNA, a trRNA, or a crRNA and trRNA. In some embodiments, the nucleotide sequence encoding the crRNA, trRNA, or crRNA and trRNA comprises or consists of a guide sequence flanked by all or a portion of a repeat sequence from a naturally occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA. In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the DNA molecule further comprises a promoter operably linked to the sequence encoding any of the ORF encoding a polypeptide disclosed herein. In some embodiments, the DNA molecule is an expression construct suitable for expression in a mammalian cell, e.g., a human cell or a mouse cell, such as a human hepatocyte or a rodent (e.g., mouse) hepatocyte. In some embodiments, the DNA molecule is an expression construct suitable for expression in a cell of a mammalian organ, e.g., a human liver or a rodent (e.g., mouse) liver. In some embodiments, the DNA molecule is a plasmid or an episome. In some embodiments, the DNA molecule is contained in a host cell, such as a bacterium or a cultured eukaryotic cell. Exemplary bacteria include proteobacteria such as E. coli. Exemplary cultured eukaryotic cells include primary hepatocytes, including hepatocytes of rodent (e.g., mouse) or human origin; hepatocyte cell lines, including hepatocytes of rodent (e.g., mouse) or human origin; human cell lines; rodent (e.g., mouse) cell lines; CHO cells; microbial fungi, such as fission or budding yeasts, e.g., Saccharomyces, such as S. cerevisiae; and insect cells.

In some embodiments, a method of producing an mRNA disclosed herein is provided. In some embodiments, such a method comprises contacting a DNA molecule described herein with an RNA polymerase under conditions permissive for transcription. In some embodiments, the contacting is performed in vitro, e.g., in a cell-free system. In some embodiments, the RNA polymerase is an RNA polymerase of bacteriophage origin, such as T7 RNA polymerase. In some embodiments, NTPs are provided that include at least one modified nucleotide as discussed above. In some embodiments, the NTPs include at least one modified nucleotide as discussed above and do not comprise UTP.

In some embodiments, a method of producing a polynucleotide disclosed herein is provided. In some embodiments, such a method comprises contacting an expression construct disclosed herein with an RNA polymerase and NTPs that comprise at least one one modified nucleotide. In some embodiments, the modified nucleotide comprises a modified uridine. In further embodiments, at least 80% of the uridine positions are modified uridines. In further embodiments, at least 90% of the uridine positions are modified uridines. In further embodiments, 100% of the uridine positions are modified uridines. In further embodiments, the modified uridine comprises or is a substituted uridine, pseudouridine, or a substituted pseudouridine. In further embodiments, the modified uridine comprises or is N1-methyl-psuedouridine. In some embodiments, the expression construct comprises an encoded poly-A tail sequence.

In some embodiments, a polynucleotide disclosed herein may be comprised within or delivered by a vector system of one or more vectors. In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences.

In some embodiments, the vector may be capable of driving expression of one or more coding sequences, such as the coding sequence of an mRNA disclosed herein, in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector system may comprise one copy of a nucleotide sequence comprising an ORF encoding a polypeptide disclosed herein. In other embodiments, the vector system may comprise more than one copy of a nucleotide sequence encoding a polypeptide disclosed herein. In some embodiments, the nucleotide sequence encoding the polypeptide disclosed herein may be operably linked to at least one transcriptional or translational control sequence. In some embodiments, the nucleotide sequence encoding the nuclease may be operably linked to at least one promoter.

In some embodiments, the promoter may be constitutive, inducible, or tissue specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding at least one guide RNA. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a ribonucleoprotein complex with the RNA-guided DNA-binding agent (e.g., NmeCas9). In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA. In other embodiments, the crRNA and the trRNA may be driven by their corresponding promoters on the same vector. In yet other embodiments, the crRNA and the trRNA may be encoded by different vectors.

In some embodiments, the compositions comprise a vector system, wherein the system comprises more than one vector. In some embodiments, the vector system may comprise one single vector. In other embodiments, the vector system may comprise two vectors. In additional embodiments, the vector system may comprise three vectors. When different polynucleotides are used for multiplexing, or when multiple copies of the polynucleotides are used, the vector system may comprise more than three vectors.

In some embodiments, a host cell is provided, the host cell comprising a vector, expression construct, or plasmid disclosed herein.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

### VI. Determination of Efficacy of ORFs

The efficacy of a polynucleotide comprising an ORF encoding a polypeptide disclosed herein may be determined when the polypeptide is expressed together with other components for a target function or system, e.g., using any of those recognized in the art to detect the presence, expression level, or activity of a particular polypeptide, e.g., by enzyme linked immunosorbent assay (ELISA), other immunological methods, western blots), liquid chromatography-mass spectrometry (LC-MS), FACS analysis, HiBiT peptide assay (Promega), or other assays described herein; or methods for determining enzymatic activity levels in biological samples (e.g., cells, cell lysates or extracts, conditioned medium, whole blood, serum, plasma, urine, or tissue), such as in vitro activity assays. Exemplary assays for activity of various encoded polypeptides described herein, e.g., RNA-guided DNA binding agents, include assays for indel formation, deamination, or mRNA or protein expression. In some embodiments, the efficacy of a polynucleotide comprising an ORF encoding a polypeptide disclosed herein is determined based on in vitro models.

### 1. Determination of Efficacy of ORFs Encoding an RNA-Guided DNA-Binding Agent

In some embodiments, the efficacy of an mRNA is determined when expressed together with other components of an RNP, e.g., at least one gRNA, such as a gRNA targeting TTR.

An RNA-guided DNA-binding agent (e.g., NmeCas9) with cleavase activity can lead to double-stranded breaks in the DNA. Nonhomologous end joining (NHEJ) is a process whereby double-stranded breaks (DSBs) in the DNA are repaired via re-ligation of the break ends, which can produce errors in the form of insertion/deletion (indel) mutations. The DNA ends of a DSB are frequently subjected to enzymatic processing, resulting in the addition or removal of nucleotides at one or both strands before the rejoining of the ends. These additions or removals prior to rejoining result in the presence of insertion or deletion (indel) mutations in the DNA sequence at the site of the NHEJ repair. Many mutations due to indels alter the reading frame or introduce premature stop codons and, therefore, produce a non-functional protein.

In some embodiments, the efficacy of an mRNA encoding a nuclease is determined based on in vitro models. In some embodiments, the in vitro model is HEK293 cells. In some embodiments, the in vitro model is HUH7 human hepatocarcinoma cells. In some embodiments, the in vitro model is primary hepatocytes, such as primary human or mouse hepatocytes.

In some embodiments, detecting gene editing events, such as the formation of insertion/deletion (“indel”) mutations utilize linear amplification with a tagged primer and isolating the tagged amplification products (herein after referred to as “LAM-PCR,” or “Linear Amplification (LA)” method, as described in WO2018/067447 or Schmidt et al., Nature Methods 4:1051-1057 (2007), or next-generation sequencing (“NGS”; e.g., using the Illumina NGS platform) as described below or other methods known in the art for detecting indel mutations.

For example, to quantitatively determine the efficiency of editing at the target location in the genome, in the NGS method, genomic DNA is isolated and deep sequencing is utilized to identify the presence of insertions and deletions introduced by gene editing. PCR primers are designed around the target site (e.g., TTR), and the genomic area of interest is amplified. Additional PCR is performed according to the manufacturer's protocols (Illumina) to add the necessary chemistry for sequencing. The amplicons are sequenced on an Illumina MiSeq instrument. The reads are aligned to the reference genome (e.g., mm10) after eliminating those having low quality scores. The resulting files containing the reads are mapped to the reference genome (BAM files), where reads that overlapped the target region of interest are selected and the number of wild type reads versus the number of reads which contain an insertion, substitution, or deletion is calculated. The editing percentage (e.g., the “editing efficiency” or “percent editing”) is defined as the total number of sequence reads with insertions or deletions over the total number of sequence reads, including wild type.

### EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated mRNA containing N1-methyl pseudo-U was generated by in vitro transcription using routine methods. For example, a plasmid DNA containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL Murine RNase inhibitor (NEB); 0.004 U/μL Inorganic E. coli pyrophosphatase (NEB); and 1×reaction buffer. TURBO DNase (Thermo Fisher) was added to a final concentration of 0.01 U/μL, and the reaction was incubated at 37° C. to remove the DNA template.

The mRNA was purified using a MegaClear Transcription Clean-up kit (Thermo Fisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 el42). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanalyzer (Agilent).

When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts. Guide RNA was chemically synthesized by commercial vendors or using standard in vitro synthesis techniques with modified nucleotides.

Streptococcus pyogenes (“Spy”) Cas9 mRNA was generated from plasmid DNA encoding an open reading frame according to SEQ ID Nos: 43-47 and 49 (see sequences in Table 39A).

### Hepatocyte Cell Preparation

Primary mouse hepatocytes (PMH), primary rat hepatocytes (PRH), primary human hepatocytes (PHH), and primary cynomolgus hepatocytes (PCH) were prepared as follows. PMH (Gibco, MCM837, unless otherwise specified), PRH (Gibco, Rs977, unless otherwise specified), PCH (In Vitro ADMET Laboratories, 10136011, unless otherwise specified), PHH (Gibco, Hu8284, unless otherwise specified) were thawed and resuspended in 50 mL Cryopreserved Hepatocyte Recovery Media (CHRM) (Invitrogen, CM7000) followed by centrifugation. Cells were resuspended in hepatocyte medium with plating supplements: Williams' E Medium Plating Supplements with FBS content (Gibco, Cat. A13450). Cells were pelleted by centrifugation, resuspended in media and plated at a density of 20,000 cells/well for PMH, and 30,000 for PHH on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation and were washed once and plated with 100 μL hepatocyte maintenance medium: Williams' E Medium (Gibco, Cat. A12176-01) plus supplement pack (Gibco, Cat. CM3000).

### HEK Cell Preparation

HEK-293 cells (ATCC, CRL-1573, unless otherwise specified) were thawed and resuspended in serum-free Dulbecco's Modified Eagle Medium (Corning #10-013-CV) with 10% FBS content (Gibco #A31605-02) and 1% Penicillin-Streptomycin (Gibco #15070063). Cells were counted and plated in Dulbecco's Modified Eagle Medium (Corning #10-013-CV) with 10% FBS content (Gibco #A31605-02) on 96-well tissue culture plate (Falcon, #353072). Plated cells were allowed to settle and adhere for 18 hours in a tissue culture incubator at 37° C. and 5% CO₂ atmosphere.

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, distearoylphosphatidylcholine (DSPC), and 1,2-dimyristoyl-rac-glycero-3-methylpolyoxyethylene glycol 2000 (PEG2k-DMG) in a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs used comprise a single RNA species such as Cas9 mRNA or a sgRNA. LNP are similarly prepared with a mixture of Cas9 mRNA and a guide RNA.

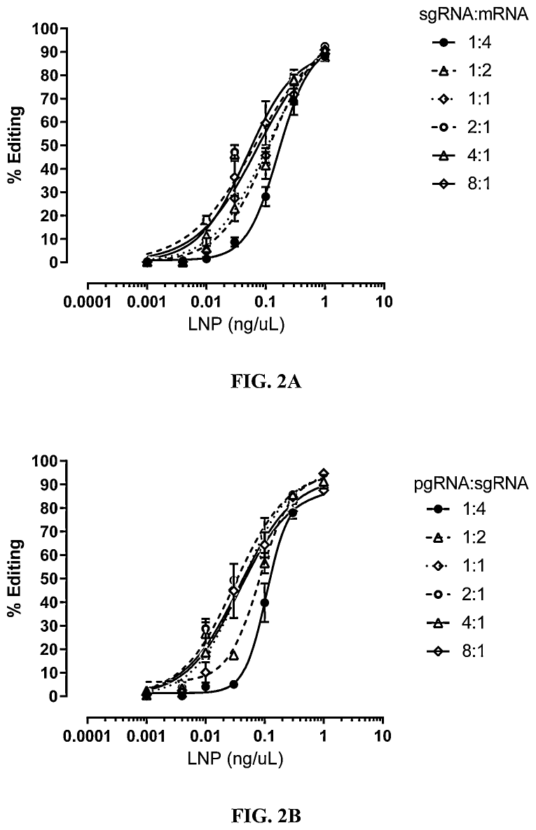

The LNPs were prepared using a cross-flow technique utilizing impinging jet mixing of the lipid in ethanol with two volumes of RNA solution and one volume of water. First, the lipid in ethanol was mixed through a mixing cross with the two volumes of RNA solution. Then, a fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNPs were characterized to determine the encapsulation efficiency, polydispersity index, and average particle size. The final LNP was stored at 4° C. or −80° C. until further use.

sgRNA and Cas9 mRNA Lipofection

Lipofection of Cas9 mRNA and gRNAs used pre-mixed lipid formulations. The lipofection reagent contained ionizable Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA (e.g., Cas9 mRNA and gRNA) at a lipid amine to RNA phosphate (N:P) molar ratio of about 6.0.

### Next-Generation Sequencing (“NGS”) and Analysis for Editing Efficiency

Genomic DNA was extracted using a commercial kit according to the manufacturer's protocol, for example QuickExtract™ DNA Extraction Solution (Lucigen, Cat. QE09050). To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations, or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 2. In Vitro Editing with Selected Guides in Primary Mouse Hepatocytes (PMH)

A modified sgRNA screen was conducted to evaluate the editing efficiency of 95 different sgRNAs targeting various sites within the mouse TTR gene. Based on that study, two sgRNAs (G021320 and G021256) were selected for evaluation in a dose response assay. These two test guides were compared to a mouse TTR SpyCas9 guide(G000502) with a 20 nucleotide guide sequence. The tested NmeCas9 sgRNAs targeting the mouse TTR gene include a 24 nucleotide guide sequence (as represented by N) and a guide scaffold as follows: mN*mNNNNNNNNmNNNmNNNNNNNNNNNNmGUUGmUmAmGmCUCCCmUmGm AmAmAmCmCGUUmGmCUAmCAAU*AAGmGmCCmGmUmCmGmAmAmAmGmAm UGUGCmCGCmAmAmCmGCUCUmGmCCmUmUmCmUGmGCmAmUC*mG*mU*mU (SEQ ID NO: 508), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guides are provided in Table 39B.

Guides and Cas9 mRNA were lipofected, as described below, into primary mouse hepatocytes (PMH). PMH (In Vitro ADMET Laboratories MCM114) were prepared as described in Example 1. Lipofections were performed as described in Example 1 with a dose response of sgRNA and mRNA. Briefly, cells were incubated at 37° C., 5% CO₂ for 24 hours prior to treatment with lipoplexes. Lipoplexes were incubated in maintenance media containing 10% fetal bovine serum (FBS) at 37° C. for 10 minutes. Post-incubation the lipoplexes were added to the mouse hepatocytes in an 8 point, 3-fold dose response assay starting at maximum dose of 300 ng Cas9 mRNA and 50 nM sgRNA. Messenger RNA doses scale along with gRNA dose in each condition, although only gRNA dose is listed in Table 5. The cells were lysed 72 hours post-treatment and NGS analysis was performed as described in Example 1.

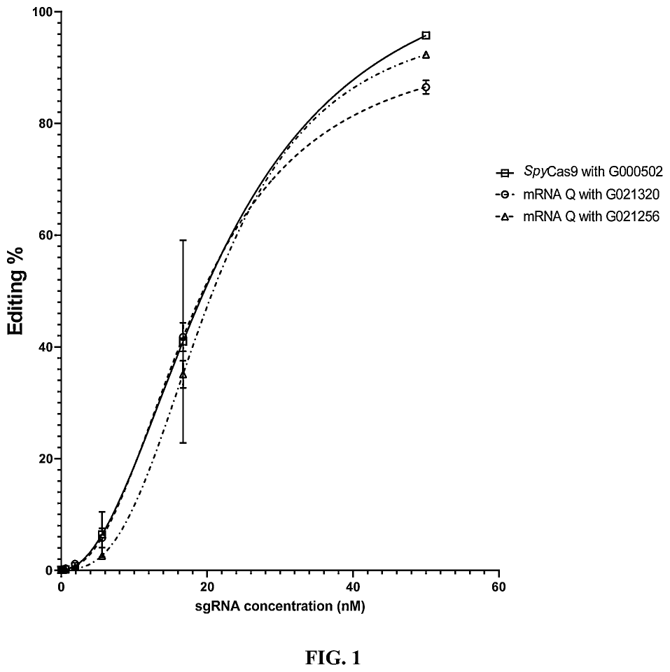

Dose response of editing efficiency to guide concentration was performed in triplicate samples. Table 5 shows mean percent editing and standard deviation (SD) at each guide concentration and a calculated EC₅₀ value. Mean and standard deviation (SD) is illustrated in FIG. 1.

TABLE 5
Mean percent editing in primary mouse hepatocytes

| | EC₅₀ | SgRNA | | |
|---|---|---|---|---|
| Sample | (nM) | (nM) | Mean % Edit | SD |
| SpyCas9 mRNA + | 22.0 | 50 | 95.7 | 0.3 |
| G000502 | | 16.7 | 40.9 | 14.8 |
| | | 5.6 | 6.4 | 3.3 |
| | | 1.9 | 0.8 | 0.3 |
| | | 0.6 | 0.2 | 0.08 |
| | | 0.2 | 0.1 | 0 |
| | | 0.1 | 0.1 | 0 |
| | | 0 | 0.1 | 0 |
| Nme2 Cas9 | 18.7 | 50 | 86.5 | 0.9 |
| mRNA Q | | 16.7 | 41.8 | 2.1 |
| SEQ ID NO: 27 + | | 5.6 | 5.8 | 1.4 |
| G021320 | | 1.9 | 1.2 | 0.3 |
| | | 0.6 | 0.4 | 0.2 |
| | | 0.2 | 0.1 | 0.1 |
| | | 0.1 | 0.1 | 0 |
| | | 0 | 0.1 | 0 |
| Nme2 Cas9 | 20.9 | 50 | 92.3 | 0.5 |
| mRNA Q | | 16.7 | 35.1 | 2 |
| SEQ ID NO: 27 + | | 5.6 | 2.6 | 0.5 |
| G021256 | | 1.9 | 0.6 | 0.4 |
| | | 0.6 | 0.1 | 0 |
| | | 0.2 | 0.1 | 0 |
| | | 0.1 | 0.1 | 0 |
| | | 0 | 0.1 | 0 |

### Example 3. sgRNA:mRNA Ratio Relative to sgRNA or pgRNA Using LNPs

Studies were conducted to evaluate the editing efficiency of sgRNA designs that contain PEG linkers (pgRNA). The study compared two gRNAs targeting TTR with the same guide sequence, one of which included three PEG linkers in the constant region of the guide (pgRNA, G021846) and one of which did not (G021845) as shown in Table 39B. The guides and mRNA were formulated in separate LNPs and mixed to the desired ratios for delivery to primary mouse hepatocytes (PMH) via lipid nanoparticles (LNPs).

PMH cells were prepared, treated, and analyzed as described in Example 1 unless otherwise noted. PMH cells from In Vitro ADMET Laboratories (Lot #MCM114) were plated at a density of 15,000 cells/well. Cells were treated with LNPs as described below. LNPs were generally prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6. LNPs encapsulated a single RNA species, either gRNA G021845, gRNA G021846 or mRNA (mRNA M) as described in Example 1.

PMH cells were treated with varying amounts of LNPs at ratios of gRNA to mRNA of 1:4, 1:2, 1:1, 2:1, 4:1, or 8:1 by weight of RNA cargo. Duplicate samples were included in each assay. Guides were assayed in an 8 point 3-fold dose response curve starting at 1 ng/μL total RNA concentration as shown in Table 6. Mean percent editing results are shown in Table 6. FIG. 2A shows mean percent editing for sgRNA G021845 and FIG. 2B shows mean percent editing for sgRNA G021846. “ND” in the table represents values that could not be detected due to experimental failure.

TABLE 6
Mean percent editing in PMH

| | | sgRNA | | pgRNA | |
|---|---|---|---|---|---|
| | | (G021845) | | (G021846) | |
| Cargo ratio | LNP dose | Mean % | | Mean % | |
| (gRNA:mRNA) | (ng/μL) | editing | SD | editing | SD |
| 1:4 | 1 | 88.1 | 1.7 | ND | ND |
| | 0.3 | 68.7 | 5.7 | 78 | 0.3 |
| | 0.1 | 28.1 | 4.1 | 39.8 | 8.2 |
| | 0.03 | 8.7 | 2 | 5.1 | 0 |
| | 0.01 | 1.5 | 0.4 | 4 | 1.2 |
| | 0.004 | 0.6 | 0.5 | 0.2 | 0 |
| | 0.001 | 0.3 | 0.2 | 0.6 | 0.3 |
| 1:2 | 1 | 90.6 | 0 | 91.2 | 2.9 |
| | 0.3 | 78 | 2.4 | 85.6 | 1.4 |
| | 0.1 | 41.5 | 5.8 | 56.6 | 4.4 |
| | 0.03 | 23 | 5.4 | 17.5 | 0 |
| | 0.01 | 6.1 | 4.3 | 18.6 | 0.5 |
| | 0.004 | 0.1 | 0.1 | 3.4 | 1.7 |
| | 0.001 | 0.1 | 0 | 2.4 | 0.7 |
| 1:1 | 1 | 90.9 | 1.4 | 94.7 | 0.6 |
| | 0.3 | 71.8 | 4.2 | 84.7 | 0.9 |
| | 0.1 | 45.7 | 3.2 | 64.3 | 5.3 |
| | 0.03 | 27.4 | 1 | 44.8 | 11.5 |
| | 0.01 | 4.7 | 2.5 | 10.2 | 4.3 |
| | 0.004 | 0.2 | 0 | 1.7 | 0.7 |
| | 0.001 | 0.1 | 0 | 0.7 | 0.5 |
| 2:1 | 1 | 92.4 | 1.6 | 94.5 | 0.8 |
| | 0.3 | 80 | 1.3 | 85.7 | 0.2 |
| | 0.1 | 45.4 | 0 | 68 | 7.9 |
| | 0.03 | 47.2 | 3 | 49.3 | 0 |
| | 0.01 | 18.1 | 1.8 | 28.8 | 4.1 |
| | 0.004 | 0.8 | 0.7 | 3.8 | 2.4 |
| | 0.001 | 0.2 | 0.1 | 0.8 | 0.3 |
| 4:1 | 1 | 87.9 | 1.9 | 90.1 | 0 |
| | 0.3 | 80.2 | 2.2 | 84 | 0.1 |
| | 0.1 | 43.4 | 0 | 60.4 | 0.1 |
| | 0.03 | 46.2 | 0.5 | 46.1 | 0 |
| | 0.01 | 11.3 | 2.3 | 26.7 | 4.9 |
| | 0.004 | 0.4 | 0.2 | 1.5 | 0.4 |
| | 0.001 | 0.4 | 0.1 | 0.5 | 0.3 |
| 8:1 | 1 | 89.2 | 0 | 87.5 | 0 |
| | 0.3 | 76.7 | 3.9 | 78.6 | 3.1 |
| | 0.1 | 59.5 | 9.4 | 59.4 | 1.1 |
| | 0.03 | 36.4 | 7 | 45.3 | 0.5 |
| | 0.01 | 8.2 | 1.2 | 18.7 | 2.9 |
| | 0.004 | 0.6 | 0.6 | 2.6 | 0.3 |
| | 0.001 | 0.1 | 0 | 0.6 | 0.2 |

### Example 3.1. In Vitro Editing of Modified Pegylated Guides (pgRNAs) in PMH Using LNPs

Modified pgRNA having the same targeting site in the mouse TTR gene were assayed to evaluate the editing efficiency in PMH cells.

PMH cells were prepared, treated, and analyzed as described in Example 1 unless otherwise noted. PMH cells from In Vitro ADMET Laboratories (Lot #MC148) were used and plated at a density of 15,000 cells/well. LNP formulations were prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6 and a gRNA indicated in Table 7 or mRNA

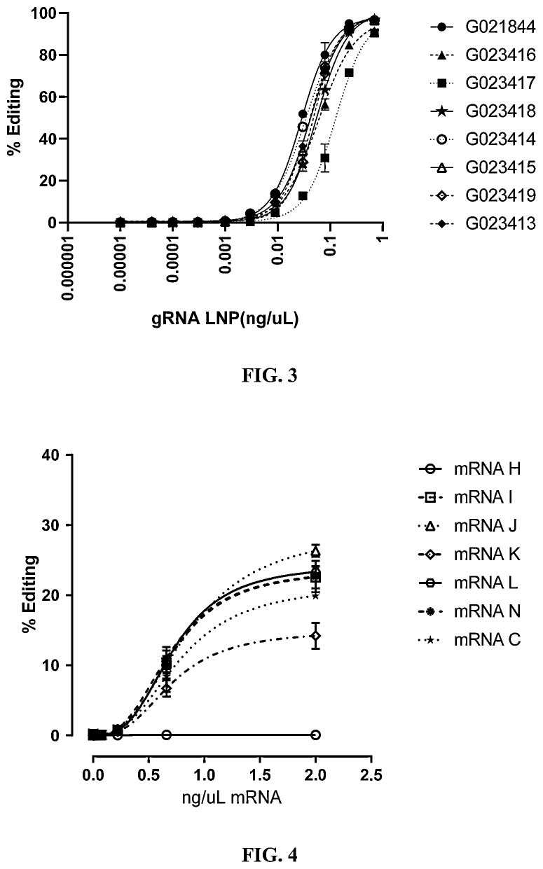

PMH in 100 μl media were treated with LNP for 30 ng total mRNA (mRNA P). by weight and LNP for gRNA in the amounts indicated in Table 7. Samples were run in duplicate. Mean editing results for PMH are shown in Table 7. and in FIG. 3.

TABLE 7
Mean percent editing in PMH

| | LNP | | |
|---|---|---|---|
| | sgRNA | Mean % | |
| Guide ID | (ng/μL) | editing | SD |
| G021844 | 0.7 | 96.6 | 0.5 |
| | 0.23 | 95.0 | 0.5 |
| | 0.08 | 80.0 | 5.9 |
| | 0.03 | 51.9 | 1.9 |
| | 0.009 | 13.9 | 0.4 |
| | 0.003 | 4.6 | 0.9 |
| | 0.001 | 0.8 | 0.1 |
| | 0.0003 | 0.2 | 0.1 |
| | 0.0001 | 0.1 | 0.0 |
| | 0.00004 | 0.2 | 0.1 |
| | 0.00001 | 0.1 | 0.0 |
| | 0 | 0.1 | 0.0 |
| G023413 | 0.7 | 96.4 | 0.4 |
| | 0.23 | 92.5 | 0.7 |
| | 0.08 | 73.9 | 0.9 |
| | 0.03 | 36.4 | 2.6 |
| | 0.009 | 10.3 | 1.5 |
| | 0.003 | 2.4 | 0.7 |
| | 0.001 | 0.6 | 0.1 |
| | 0.0003 | 0.3 | 0.0 |
| | 0.0001 | 0.1 | 0.0 |
| | 0.00004 | 0.1 | 0.0 |
| | 0.00001 | 0.1 | 0.0 |
| | 0 | 0.1 | 0.0 |
| G023414 | 0.7 | 96.5 | 0.2 |
| | 0.23 | 92.7 | 0.4 |
| | 0.08 | 74.1 | 2.7 |
| | 0.03 | 45.7 | 1.5 |
| | 0.009 | 13.7 | 0.7 |
| | 0.003 | 4.3 | 1.3 |
| | 0.001 | 0.7 | 0.1 |
| | 0.0003 | 0.2 | 0.0 |
| | 0.0001 | 0.2 | 0.1 |
| | 0.00004 | 0.2 | 0.1 |
| | 0.00001 | 0.1 | 0.0 |
| | 0 | 0.1 | 0.0 |
| G023415 | 0.7 | 96.5 | 0.5 |
| | 0.23 | 92.6 | 0.7 |
| | 0.08 | 73.1 | 0.2 |
| | 0.03 | 34.4 | 0.8 |
| | 0.009 | 14.2 | 0.2 |
| | 0.003 | 3.9 | 0.4 |
| | 0.001 | 0.5 | 0.2 |
| | 0.0003 | 0.2 | 0.0 |
| | 0.0001 | 0.2 | 0.0 |
| | 0.00004 | 0.1 | 0.0 |
| | 0.00001 | 0.1 | 0.0 |
| | 0 | 0.1 | 0.0 |
| G023416 | 0.7 | 91.5 | 1.8 |
| | 0.23 | 84.8 | 0.1 |
| | 0.08 | 56.4 | 2.7 |
| | 0.03 | 28.2 | 3.6 |
| | 0.009 | 10.0 | 1.7 |
| | 0.003 | 3.2 | 0.0 |
| | 0.001 | 0.8 | 0.3 |
| | 0.0003 | 0.2 | 0.0 |
| | 0.0001 | 0.1 | 0.0 |
| | 0.00004 | 0.1 | 0.0 |
| | 0.00001 | 0.1 | 0.0 |
| | 0 | 0.1 | 0.0 |
| G023417 | 0.7 | 90.5 | 1.8 |
| | 0.23 | 71.6 | 0.2 |
| | 0.08 | 30.9 | 6.7 |
| | 0.03 | 12.8 | 1.3 |
| | 0.009 | 4.8 | 1.5 |
| | 0.003 | 0.4 | 0.4 |
| | 0.001 | 0.2 | 0.1 |
| | 0.0003 | 0.1 | 0.0 |
| | 0.0001 | 0.1 | 0.1 |
| | 0.00004 | 0.1 | 0.0 |
| | 0.00001 | 0.1 | 0.0 |
| | 0 | 0.1 | 0.0 |
| G023418 | 0.7 | 96.8 | 0.3 |
| | 0.23 | 90.8 | 1.7 |
| | 0.08 | 63.3 | 1.8 |
| | 0.03 | 27.7 | 2.4 |
| | 0.009 | 8.8 | 0.5 |
| | 0.003 | 1.9 | 0.6 |
| | 0.001 | 0.7 | 0.2 |
| | 0.0003 | 0.2 | 0.1 |
| | 0.0001 | 0.1 | 0.0 |
| | 0.00004 | 0.1 | 0.0 |
| | 0.00001 | 0.1 | 0.0 |
| | 0 | 0.2 | 0.1 |
| G023419 | 0.7 | 96.6 | 0.6 |
| | 0.23 | 93.4 | 1.3 |
| | 0.08 | 71.1 | 3.3 |
| | 0.03 | 29.0 | 4.6 |
| | 0.009 | 9.7 | 4.1 |
| | 0.003 | 2.3 | 0.5 |
| | 0.001 | 0.4 | 0.0 |
| | 0.0003 | 0.1 | 0.0 |
| | 0.0001 | 0.2 | 0.0 |
| | 0.00004 | 0.2 | 0.0 |
| | 0.00001 | 0.1 | 0.0 |
| | 0 | 0.1 | 0.0 |

### Example 4. Nme2-mRNA Studies

### Example 4.1—In Vitro Editing in Primary Mouse Hepatocytes

Messenger mRNAs encoding Nme2Cas9 ORFs with different NLS placements were assayed for editing efficiency in primary mouse hepatocytes (PMH).

PMH were prepared as described in Example 1. Lipofection was performed using Lipofectamine MessengerMAX Transfection Reagent (Invitrogen LMRNA001) according to the manufacturer's protocol to transform cells with 100 nM sgRNA G020361 targeting mouse PCSK9 and with mRNA at the concentrations listed in Table 8. Triplicate samples were included in the assay. After 72 hours incubation at 37° C. in Maintenance Media, cells were harvested and NGS analysis was performed as described in Example 1. Mean editing results with standard deviation (SD) are shown in Table 8 and FIG. 4.

TABLE 8
Mean editing percentage in at the PCSK9 locus in PMH

| | mRNA Concentration | Mean % | |
|---|---|---|---|
| Construct | (ng/uL) | editing | SD |
| mRNA H | 2.00 | 0.07 | 0.05 |
| | 0.66 | 0.07 | 0.05 |
| | 0.22 | 0.03 | 0.05 |
| | 0.07 | 0.03 | 0.05 |
| | 0.03 | 0.03 | 0.05 |
| | 0.008 | 0.00 | 0.00 |
| | 0.003 | 0.00 | 0.00 |
| | 0.00 | 0.05 | 0.05 |
| mRNA I | 2.00 | 22.53 | 1.59 |
| | 0.66 | 10.37 | 2.25 |
| | 0.22 | 0.80 | 0.22 |
| | 0.07 | 0.07 | 0.05 |
| | 0.03 | 0.07 | 0.05 |
| | 0.008 | 0.03 | 0.05 |
| | 0.003 | 0.03 | 0.05 |
| | 0.00 | 0.20 | 0.28 |
| mRNA J | 2.00 | 26.30 | 0.86 |
| | 0.66 | 10.07 | 1.27 |
| | 0.22 | 0.93 | 0.33 |
| | 0.07 | 0.03 | 0.05 |
| | 0.03 | 0.03 | 0.05 |
| | 0.008 | 0.03 | 0.05 |
| | 0.003 | 0.03 | 0.05 |
| | 0.00 | 0.05 | 0.05 |
| mRNA K | 2.00 | 14.20 | 1.84 |
| | 0.66 | 6.70 | 1.16 |
| | 0.22 | 0.53 | 0.17 |
| | 0.07 | 0.07 | 0.09 |
| | 0.03 | 0.03 | 0.05 |
| | 0.008 | 0.00 | 0.00 |
| | 0.003 | 0.00 | 0.00 |
| | 0.00 | 0.05 | 0.05 |
| mRNA L | 2.00 | 23.30 | 0.80 |
| | 0.66 | 10.57 | 1.54 |
| | 0.22 | 0.70 | 0.42 |
| | 0.7 | 0.07 | 0.05 |
| | 0.03 | 0.07 | 0.05 |
| | 0.008 | 0.07 | 0.05 |
| | 0.003 | 0.03 | 0.05 |
| | 0.00 | 0.05 | 0.05 |
| mRNA N | 2.00 | 22.63 | 2.25 |
| | 0.66 | 11.00 | 0.00 |
| | 0.22 | 0.97 | 0.19 |
| | 0.07 | 0.17 | 0.09 |
| | 0.03 | 0.03 | 0.05 |
| | 0.008 | 0.03 | 0.05 |
| | 0.003 | 0.00 | 0.00 |
| | 0.00 | 0.05 | 0.05 |
| mRNA C | 2.00 | 19.90 | 0.16 |
| | 0.66 | 8.40 | 2.20 |
| | 0.22 | 0.70 | 0.22 |
| | 0.07 | 0.03 | 0.05 |
| | 0.03 | 0.00 | 0.00 |
| | 0.008 | 0.03 | 0.05 |
| | 0.003 | 0.00 | 0.00 |
| | 0.00 | 0.00 | 0.00 |

### Example 4.2—Dose Response of Nme2 ORF Variants and Guides with Chemical Modification Variations

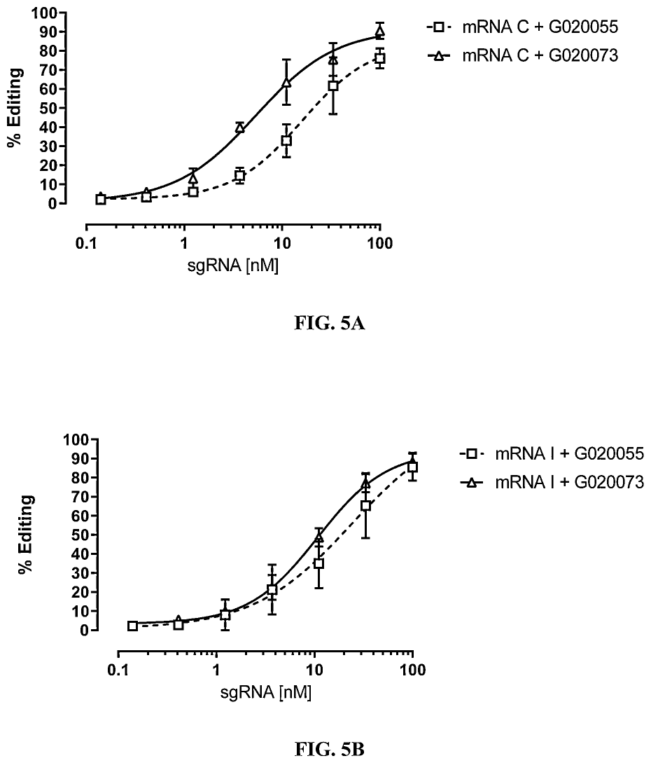

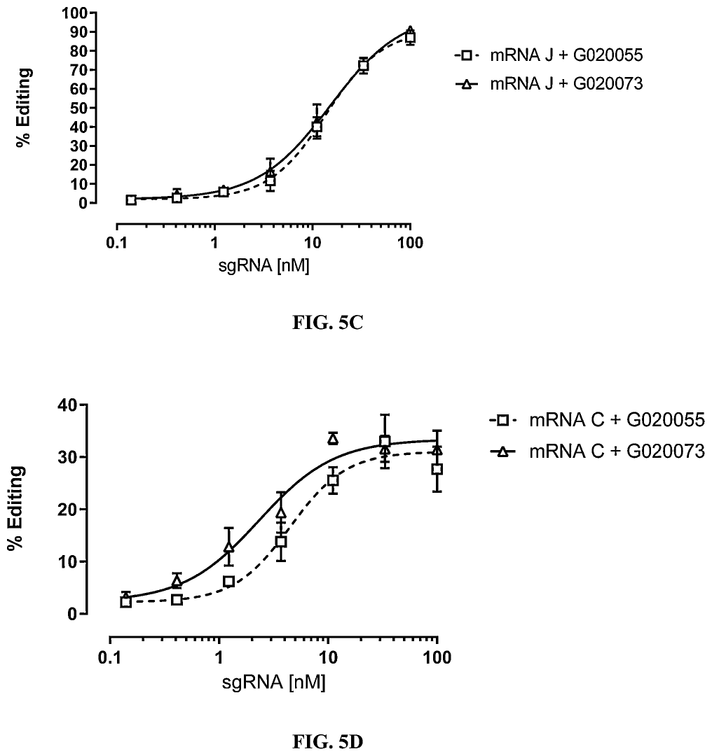

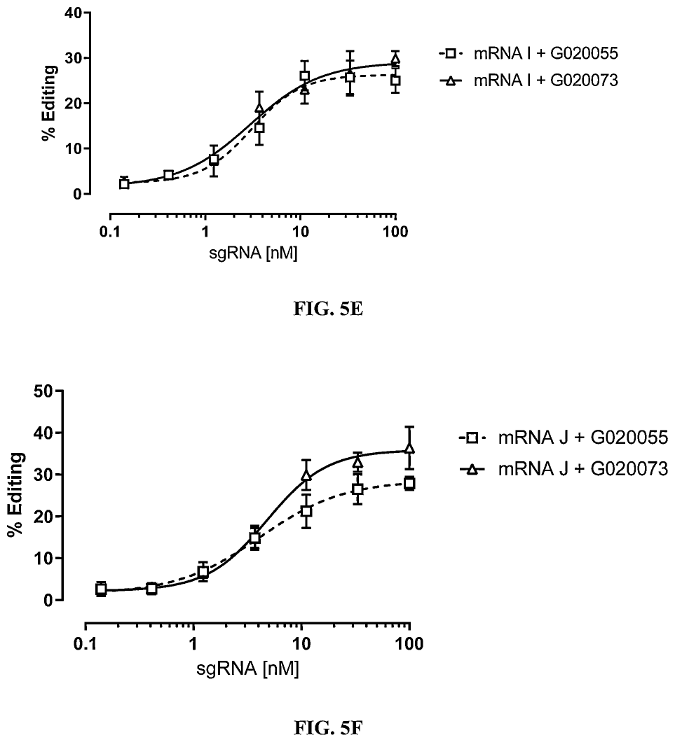

Messenger mRNAs encoding Nme2Cas9 ORFs with different NLS configurations were assayed for editing efficiency in primary human hepatocytes (PHH) and HEK-293 cells. Assays were performed using gRNAs with identical guide sequences targeting VEGFA locus TS47 and gRNAs had various lengths and chemical modification patterns. PHH cells prepared as described in Example 1. HEK293 cells were thawed and plated at a density of 30,000 cells/well in 96 well plates in DMEM (Corning, 10-013-CV) with 10% FBS and incubated for 24 hours. Lipofection was performed using Lipofectamine MessengerMAX Transfection Reagent (Invitrogen LMRNA001) according to the manufacturer's protocol. A dose response 1:3 dilution series starting at atop dose of 100 nM gRNA and 1 ng/μL mRNA, was used to transform cells with gRNA at the concentrations listed in Tables 9-10. Replicate samples were included in the assay. After 72 hours incubation at 37° C., cells were harvested and NGS analysis was performed as described in Example 1. Mean editing results with standard deviation (SD) are shown in Table 9 and FIG. 5A-5C for HEK cells and Table 10 and FIG. 5D-5F for PHH.

TABLE 9
Mean percent editing in HEK cells

| | gRNA | G020055 | | | G020073 | | |
|---|---|---|---|---|---|---|---|
| mRNA | [nM] | Mean | SD | N | Mean | SD | N |
| mRNA C | 100 | 76.05 | 5.18 | 4 | 90.50 | 4.15 | 4 |
| SEQ ID NO: | 33.33 | 61.68 | 14.86 | 4 | 75.55 | 8.65 | 4 |
| 15 | 11.11 | 32.93 | 8.59 | 4 | 63.53 | 11.92 | 4 |
| | 3.70 | 14.63 | 4.08 | 4 | 39.88 | 2.40 | 4 |
| | 1.23 | 5.95 | 1.42 | 4 | 13.00 | 5.36 | 4 |
| | 0.41 | 3.35 | 0.60 | 4 | 6.03 | 1.05 | 4 |
| | 0.14 | 2.05 | 0.50 | 4 | 3.65 | 0.47 | 4 |
| | 0.00 | 1.50 | 0.22 | 4 | 1.30 | 0.22 | 4 |
| mRNA I | 100 | 85.55 | 7.06 | 4 | 88.08 | 4.90 | 4 |
| SEQ ID | 33.33 | 65.33 | 17.06 | 4 | 77.13 | 4.78 | 4 |
| NO: 19 | 11.11 | 34.98 | 12.93 | 4 | 48.63 | 4.83 | 4 |
| | 3.70 | 21.25 | 13.09 | 4 | 22.43 | 6.49 | 4 |
| | 1.23 | 8.03 | 8.06 | 4 | 9.80 | 2.12 | 4 |
| | 0.41 | 2.83 | 1.94 | 4 | 5.55 | 0.94 | 4 |
| | 0.14 | 2.15 | 0.83 | 4 | 2.45 | 0.60 | 4 |
| mRNA J | 100 | 87.03 | 3.79 | 4 | 90.93 | 1.14 | 4 |
| SEQ ID NO: | 33.33 | 72.25 | 4.18 | 4 | 72.08 | 3.88 | 4 |
| 20 | 11.11 | 40.05 | 5.01 | 4 | 42.83 | 9.02 | 4 |
| | 3.70 | 11.65 | 5.34 | 4 | 16.63 | 6.64 | 4 |
| | 1.23 | 5.78 | 0.86 | 4 | 7.00 | 1.47 | 4 |
| | 0.41 | 2.60 | 0.42 | 4 | 4.50 | 2.76 | 4 |
| | 0.14 | 1.53 | 0.50 | 4 | 1.78 | 0.24 | 4 |
| | 0.00 | 1.33 | 0.13 | 4 | 1.53 | 0.25 | 4 |

TABLE 10
Mean percent editing in PHH cells

| | | G020055 | | | G020073 | | |
|---|---|---|---|---|---|---|---|
| mRNA | gRNA [nM] | Mean | SD | N | Mean | SD | N |
| mRNA C | 100 | 27.70 | 4.29 | 3 | 31.43 | 3.63 | 3 |
| | 33.33 | 32.98 | 5.10 | 4 | 31.58 | 2.49 | 4 |
| | 11.11 | 25.55 | 2.53 | 4 | 33.58 | 1.06 | 4 |
| | 3.70 | 13.80 | 3.68 | 4 | 19.38 | 3.86 | 4 |
| | 1.23 | 6.20 | 0.68 | 4 | 12.83 | 3.60 | 4 |
| | 0.41 | 2.70 | 0.81 | 4 | 6.35 | 1.41 | 4 |
| | 0.14 | 2.25 | 0.66 | 4 | 3.18 | 1.05 | 4 |
| | 0.00 | 1.65 | 0.24 | 4 | 1.50 | 0.18 | 4 |
| mRNA I | 100 | 25.00 | 2.73 | 4 | 29.88 | 1.67 | 4 |
| | 33.33 | 25.73 | 3.69 | 4 | 26.60 | 4.95 | 4 |
| | 11.11 | 26.08 | 3.23 | 4 | 22.98 | 3.09 | 4 |
| | 3.70 | 14.55 | 3.74 | 4 | 19.03 | 3.55 | 4 |
| | 1.23 | 7.65 | 0.70 | 4 | 7.28 | 3.41 | 4 |
| | 0.41 | 4.18 | 0.97 | 4 | 4.15 | 0.62 | 4 |
| | 0.14 | 2.18 | 0.15 | 4 | 2.83 | 0.93 | 4 |
| | 0.00 | 1.40 | 0.12 | 4 | 1.35 | 0.17 | 4 |
| mRNA J | 100 | 27.90 | 1.57 | 4 | 36.38 | 5.06 | 4 |
| | 33.33 | 26.50 | 3.59 | 4 | 32.95 | 2.27 | 4 |
| | 11.11 | 21.23 | 3.98 | 4 | 29.88 | 3.59 | 4 |
| | 3.70 | 14.85 | 2.37 | 4 | 14.88 | 2.81 | 4 |
| | 1.23 | 6.78 | 2.29 | 4 | 6.43 | 0.74 | 4 |
| | 0.41 | 2.73 | 1.35 | 4 | 3.13 | 0.49 | 4 |
| | 0.14 | 2.63 | 1.69 | 4 | 2.45 | 0.73 | 4 |
| | 0.00 | 1.40 | 0.12 | 4 | 1.50 | 0.16 | 4 |

### Example 4.3—Dose Response of Nme2 NLS Variants Using LNPs in PMH

Messenger mRNAs encoding Nme2Cas9 ORFs with different NLS placements were assayed for editing efficiency in primary mouse hepatocytes (PMH). The assay tested guides targeting the mouse TTR locus and included both sgRNA and pgRNA designs.

PMH were prepared as in Example 1. LNPs were generally prepared as described in Example 1 with a single RNA species as cargo, as indicated in Table 11. LNPs were prepared with the lipid composition of 50/9/38/3, expressed as the molar ratio of ionizable Lipid A/cholesterol/DSPC/PEG, respectively. The LNPs were formulated with a lipid amine to RNA phosphate (N:P) molar ratio of about 6.

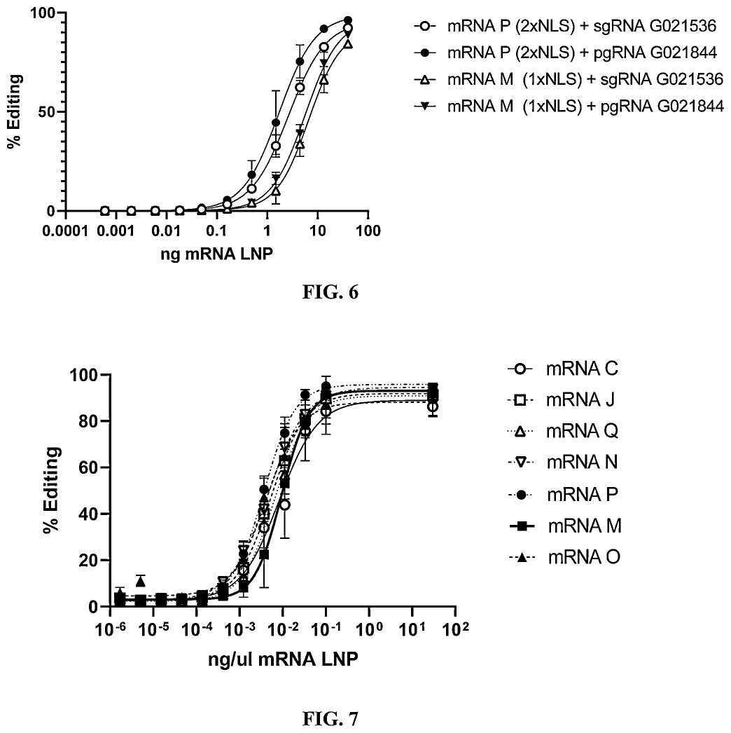

Cells were treated with 60 ng/100 μl LNP containing gRNA by RNA weight and with LNP containing mRNA as indicated in Table 11. Cells were incubated for 72 hours at 37° C. in Williams' E Medium (Gibco, A1217601) with maintenance supplements and 10% fetal bovine serum. After 72 hours incubation at 37° C., cells were harvested and editing was assessed by NGS as described in Example 1. Mean percent editing data is shown in Table 11 and FIG. 6.

TABLE 11
Mean percent editing at the mouse TTR
locus in primary mouse hepatocytes.

| | mRNA LNP | Mean % | | |
|---|---|---|---|---|
| Sample | (ng RNA) | Editing | SD | N |
| mRNA P (2 × NLS) | 40.000 | 92.30 | 0.85 | 3 |
| G021536 | 13.330 | 82.67 | 1.61 | 3 |
| | 4.440 | 62.27 | 2.96 | 3 |
| | 1.480 | 32.80 | 4.54 | 3 |
| | 0.490 | 11.23 | 1.37 | 3 |
| | 0.160 | 3.40 | 0.71 | 3 |
| | 0.050 | 0.80 | 0.22 | 3 |
| | 0.018 | 0.30 | 0.08 | 3 |
| | 0.006 | 0.20 | 0.08 | 3 |
| | 0.002 | 0.13 | 0.05 | 3 |
| | 0.001 | 0.13 | 0.05 | 3 |
| | 0.000 | 0.10 | 0.00 | 3 |
| mRNA P (2 × NLS) | 40.000 | 96.17 | 0.12 | 3 |
| G021844 (pgRNA) | 13.330 | 91.83 | 0.34 | 3 |
| | 4.440 | 75.37 | 6.80 | 3 |
| | 1.480 | 44.53 | 13.11 | 3 |
| | 0.490 | 18.30 | 5.77 | 3 |
| | 0.160 | 5.50 | 1.43 | 3 |
| | 0.050 | 1.63 | 0.71 | 3 |
| | 0.018 | 0.33 | 0.05 | 3 |
| | 0.006 | 0.17 | 0.05 | 3 |
| | 0.002 | 0.07 | 0.05 | 3 |
| | 0.001 | 0.10 | 0.00 | 3 |
| | 0.000 | 0.07 | 0.05 | 3 |
| mRNA M (1 × NLS) | 40.000 | 84.27 | 1.23 | 3 |
| G021536 | 13.330 | 66.23 | 5.39 | 3 |
| | 4.440 | 33.80 | 5.14 | 3 |
| | 1.480 | 10.17 | 5.51 | 3 |
| | 0.490 | 4.20 | 0.92 | 3 |
| | 0.160 | 1.10 | 0.45 | 3 |
| | 0.050 | 0.33 | 0.17 | 3 |
| | 0.018 | 0.23 | 0.09 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.07 | 0.05 | 3 |
| mRNA M (1 × NLS) | 40.000 | 88.83 | 0.37 | 3 |
| G021844 (pgRNA) | 13.330 | 74.37 | 4.63 | 3 |
| | 4.440 | 39.00 | 3.72 | 3 |
| | 1.480 | 16.40 | 2.52 | 3 |
| | 0.490 | 4.03 | 0.77 | 3 |
| | 0.160 | 1.27 | 0.05 | 3 |
| | 0.050 | 0.23 | 0.05 | 3 |
| | 0.018 | 0.20 | 0.08 | 3 |
| | 0.006 | 0.10 | 0.00 | 3 |
| | 0.002 | 0.10 | 0.00 | 3 |
| | 0.001 | 0.10 | 0.00 | 3 |
| | 0.000 | 0.10 | 0.00 | 3 |

### Example 4.4—Dose Response of Nme2 NLS Variants Using LNPs in PMH

Messenger mRNAs encoding Nme2Cas9 ORFs with different NLS placements were assayed for editing efficiency in primary mouse hepatocytes (PMH).

PMH (Gibco, MC148) were prepared as described in Example 1. LNPs were generally prepared as described in Example 1 with a single RNA species as cargo. LNPs were prepared with the lipid composition of 50/9/38/3, expressed as the molar ratio of ionizable Lipid A/cholesterol/DSPC/PEG, respectively. The LNPs were formulated with a lipid amine to RNA phosphate (N:P) molar ratio of about 6.

Cells were treated with 30 ng by RNA weight/100 μl of LNP containing gRNA G021844 and with LNP containing mRNA as indicated in Table LS4. Cells were incubated for 24 hours in Williams' E Medium (Gibco, A1217601) with maintenance supplements and 10% fetal bovine serum. After 72 hours incubation, cells were harvested and editing was assessed by NGS as described in Example 1. Mean percent editing data is shown in Table 12 and FIG. 7.

TABLE 12
Mean editing percentage in PMH treated with LNPs.

| | mRNA LNP | | | | EC₅₀ |
|---|---|---|---|---|---|
| mRNA | (ng/uL) | Mean | SD | N | (ng/uL) |
| mRNA C | 0.30 | 86.30 | 4.46 | 3 | 0.0082 |
| SEQ ID NO: | 0.10 | 84.17 | 5.52 | | |
| 15 | 0.03 | 75.80 | 1.91 | | |
| | 0.01 | 43.90 | 14.36 | | |
| | 0.004 | 34.03 | 8.64 | | |
| | 0.001 | 15.63 | 4.35 | | |
| | 0.0004 | 6.17 | 2.41 | | |
| | 0.0001 | 3.47 | 0.62 | | |
| | 0.00005 | 2.37 | 0.34 | | |
| | 0.00002 | 3.00 | 0.64 | | |
| | 0.00001 | 2.60 | 0.57 | | |
| | 0.00 | 2.70 | 0.16 | | |
| mRNA J | 0.30 | 91.30 | 2.92 | 3 | 0.0053 |
| SEQ ID NO: | 0.10 | 89.60 | 4.23 | | |
| 20 | 0.03 | 80.93 | 8.17 | | |
| | 0.01 | 62.85 | 14.35 | | |
| | 0.004 | 39.95 | 5.15 | | |
| | 0.001 | 16.70 | 3.79 | | |
| | 0.0004 | 7.73 | 2.98 | | |
| | 0.0001 | 4.23 | 0.95 | | |
| | 0.00005 | 2.80 | 0.70 | | |
| | 0.00002 | 3.23 | 0.54 | | |
| | 0.00001 | 2.67 | 0.48 | | |
| | 0.00 | 3.60 | 0.57 | | |
| mRNA Q | 0.30 | 90.67 | 4.40 | 3 | 0.0065 |
| SEQ ID NO: | 0.10 | 86.77 | 5.43 | | |
| 27 | 0.03 | 80.27 | 6.65 | | |
| | 0.01 | 56.90 | 5.48 | | |
| | 0.004 | 35.45 | 1.35 | | |
| | 0.001 | 12.63 | 3.16 | | |
| | 0.0004 | 5.17 | 0.56 | | |
| | 0.0001 | 2.73 | 0.17 | | |
| | 0.00005 | 2.97 | 0.41 | | |
| | 0.00002 | 2.73 | 0.21 | | |
| | 0.00001 | 2.87 | 0.56 | | |
| | 0.00 | 2.43 | 0.82 | | |
| mRNA N | 0.30 | 93.93 | 2.20 | 3 | 00045 |
| SEQ ID NO: | 0.10 | 90.97 | 1.77 | | |
| 24 | 0.03 | 82.80 | 8.24 | | |
| | 0.01 | 68.67 | 10.18 | | |
| | 0.004 | 42.07 | 2.25 | | |
| | 0.001 | 24.13 | 4.21 | | |
| | 0.0004 | 10.60 | 0.94 | | |
| | 0.0001 | 4.67 | 0.66 | | |
| | 0.00005 | 3.30 | 1.84 | | |
| | 0.00002 | 3.37 | 0.69 | | |
| | 0.00001 | 2.53 | 0.90 | | |
| | 0.00 | 2.33 | 1.48 | | |
| mRNA P | 0.30 | 94.47 | 1.04 | 3 | 0.0036 |
| SEQ ID NO: | 0.10 | 95.03 | 0.96 | | |
| 26 | 0.03 | 91.27 | 2.36 | | |
| | 0.01 | 74.77 | 6.91 | | |
| | 0.004 | 50.57 | 4.89 | | |
| | 0.001 | 22.67 | 0.25 | | |
| | 0.0004 | 8.27 | 0.74 | | |
| | 0.0001 | 4.93 | 0.70 | | |
| | 0.00005 | 3.37 | 0.74 | | |
| | 0.00002 | 2.93 | 0.68 | | |
| | 0.00001 | 2.87 | 0.05 | | |
| | 0.00 | 2.87 | 0.45 | | |
| mRNA M | 0.30 | 92.00 | 0.80 | 3 | 0.0093 |
| SEQ ID NO: | 0.10 | 91.40 | 1.90 | | |
| 23 | 0.03 | 79.70 | 0.70 | | |
| | 0.01 | 53.10 | 6.80 | | |
| | 0.004 | 22.47 | 14.28 | | |
| | 0.001 | 8.20 | 4.20 | | |
| | 0.0004 | 4.57 | 1.57 | | |
| | 0.0001 | 2.73 | 0.31 | | |
| | 0.00005 | 3.07 | 0.21 | | |
| | 0.00002 | 2.93 | 0.09 | | |
| | 0.00001 | 2.77 | 0.66 | | |
| | 0.00 | 3.47 | 1.09 | | |
| mRNA O | 0.30 | 89.40 | 7.00 | 3 | 0.0042 |
| SEQ ID NO: | 0.10 | 86.83 | 12.52 | | |
| 25 | 0.03 | 78.17 | 15.41 | | |
| | 0.01 | 64.83 | 12.48 | | |
| | 0.004 | 47.33 | 9.03 | | |
| | 0.001 | 20.67 | 7.12 | | |
| | 0.0004 | 8.60 | 2.95 | | |
| | 0.0001 | 2.47 | 1.33 | | |
| | 0.00005 | 4.13 | 0.37 | | |
| | 0.00002 | 2.80 | 0.62 | | |
| | 0.00001 | 11.13 | 231. | | |
| | 0.00 | 6.13 | 2.16 | | |

### Example 5—NmeCas9 Protein Expression

### Example 5.1 Protein Expression in Primary Human Hepatocytes

To quantify expression of each mRNA construct, mRNA and protein expression levels were measured following LNP delivery of mRNAs encoding either SpyCas9 or NmeCas9 to primary human hepatocytes.

PHH cells were prepared as described in Example 1. LNPs were generally prepared as described in Example 1 with a single RNA species as cargo. The LNPs were made with a molar ratio of 50% Lipid A, 38% cholesterol, 9% DSPC, and 3% PEG2k-DMG. The LNPs were formulated with a lipid amine to RNA phosphate (N:P) molar ratio of about 6.

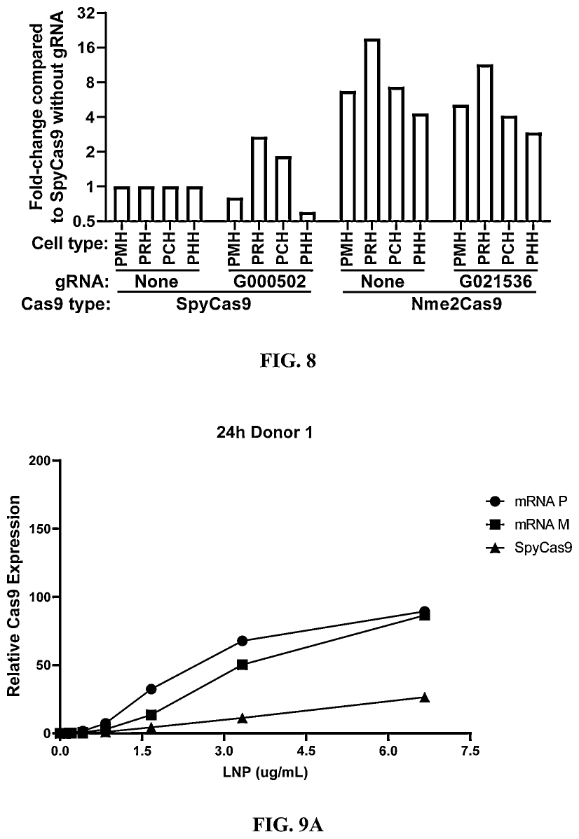

Cells were dosed with one LNP containing mRNA (mRNA only), or two LNPs containing either mRNA or gRNA. Each LNP was applied to cells at 16.7 ng total RNA cargo/100 μl. Upon treatment with LNPs, cells were incubated for 24 hours in Williams' E Medium (Gibco, A1217601) with maintenance supplements and 10% fetal bovine serum. After 24 hours incubation at 37° C., cells were harvested and expression was quantified via Nano-Glo HiBiT lytic detection system (Promega, N3030) following manufacturer's instructions. Raw luminescence was normalized to a standard curve using HiBiT Control Protein (Promega, N3010). Protein expression of different Cas9 variants, shown in Table 13 and FIG. 8, was normalized to the expression of SpyCas9 measured in corresponding hepatocytes delivered with only the SpyCas9 mRNA. Consistent with the data shown in Table 13, protein expression from these same constructs was higher for the NmeCas9 construct than for the SpyCas9 construct when detected by western blot with an anti-HiBiT antibody from PHH cell extracts or as measured by HiBiT detection in PMH, PCH, PHH, and PRH cells.

TABLE 13
Mean fold-expression of Cas9 variants as compared to SpyCas9
expression in corresponding hepatocytes delivered with
only the SpyCas9 mRNA, as measured by the HiBiT assay

Fold-expression

| mRNA | gRNA | Cell Type | Mean | N |
|---|---|---|---|---|
| Spy Cas9 | None | PMH | 1 | 2 |
| mRNA | | PRH | 1 | 3 |
| | | PCH | 1 | 3 |
| | | PHH | 1 | 3 |
| | G000502 | PMH | 0.8 | 2 |
| | | PRH | 2.7 | 3 |
| | | PCH | 1.8 | 3 |
| | | PHH | 0.6 | 3 |
| Nme2 Cas9 | None | PMH | 6.8 | 2 |
| mRNA M | | PRH | 19.2 | 3 |
| | | PCH | 7.3 | 3 |
| | | PHH | 4.3 | 3 |
| | G021536 | PMH | 5.1 | 2 |
| | | PRH | 11.5 | 3 |
| | | PCH | 4.1 | 3 |
| | | PHH | 3.0 | 3 |

### Example 5.2: Protein Expression in T Cells

To quantify expression of each mRNA construct, protein expression levels were measured following LNP delivery of mRNAs encoding either SpyCas9 or Nme2Cas9 to T Cells.

Healthy human donor apheresis was obtained commercially (Hemacare). T cells from two donors (W106 and W864) were isolated by negative selection using the EasySep Human T cell Isolation Kit (Stem Cell Technology, Cat. 17951) on the MultiMACS Cell24 Separator Plus instrument according to manufacturer instruction. Isolated T cells were cryopreserved in CS10 freezing media (Cryostor, Cat., 07930) for future use.

Upon thaw, T cells were cultured in complete T cell growth media composed of CTS OpTmizer Base Media (CTS OpTmizer Media (Gibco, A1048501) with 1×GlutaMAX, 10 mM HEPES buffer, 1% Penicillin/Streptomycin)) supplemented with cytokines (200 IU/ml IL2, 5 ng/ml IL7 and 5 ng/ml IL15) and 2.5% human serum (Gemini, 100-512). After overnight rest at 37° C., T cells at a density of 1e6/mL were activated with T cell TransAct Reagent (1:100 dilution, Miltenyi) and incubated in a tissue culture incubator for 48 hours.

The activated T cells were treated with LNPs delivering mRNAs encoding Nme2-mRNA or Spy mRNA with HiBiT tags. LNPs were generally prepared as in Example 1. LNPs were formulated with a lipid amine to RNA phosphate (N:P) molar ratio of about 6. The LNPs encapsulating Nme2Cas9 mRNAs used Lipid A, cholesterol, DSPC, and PEG2k-DMG in a molar ratio of 50% Lipid A, 38% cholesterol, 9% DSPC, and 3% PEG2k-DMG. The LNP encapsulating SpyCas9 mRNA used Lipid A, cholesterol, DSPC, and PEG2k-DMG in a molar ratio of 50% Lipid A, 38.5% cholesterol, 10% DSPC, and 1.5% PEG2k-DMG.

Immediately prior to LNP treatment of T cells, LNPs were preincubated at 37° C. for 5 minutes at an LNP concentration of 13.33 ug/ml total RNA with 10 ug/mL ApoE3 (Peprotech, Cat #350-02) in complete T cell media supplemented with cytokines (200 IU/ml IL2 (Peprotech, Cat. 200-02), 5 ng/ml IL7 (Peprotech, Cat. 200-07), and 5 ng/ml IL15 (Peprotech, Cat. 200-15) and 2.5% human serum (Gemini, 100-512). After incubation, LNPs were then mixed 1:1 by volume with T cells in the complete T cell media with cytokines used for ApoE incubation. T cells were harvested for protein expression analysis at 24 h, 48 h, and 72 h post LNP treatment. T cells were lysed by Nano-Glo® HiBiT Lytic Assay (Promega) and Cas9 protein levels quantified via Nano-Glo® Nano-Glo HiBiT Extracellular Detection System (Promega, Cat. N2420) following the manufacturer's instructions. Luminescence was measured using the Biotek Neo2 plate reader. Linear regression was plotted on GraphPad using the protein number and luminescence readouts from the standard controls, forcing the line to go through X=0, Y=0. Used the Y=ax+0 equation to calculate number of proteins per lysate.

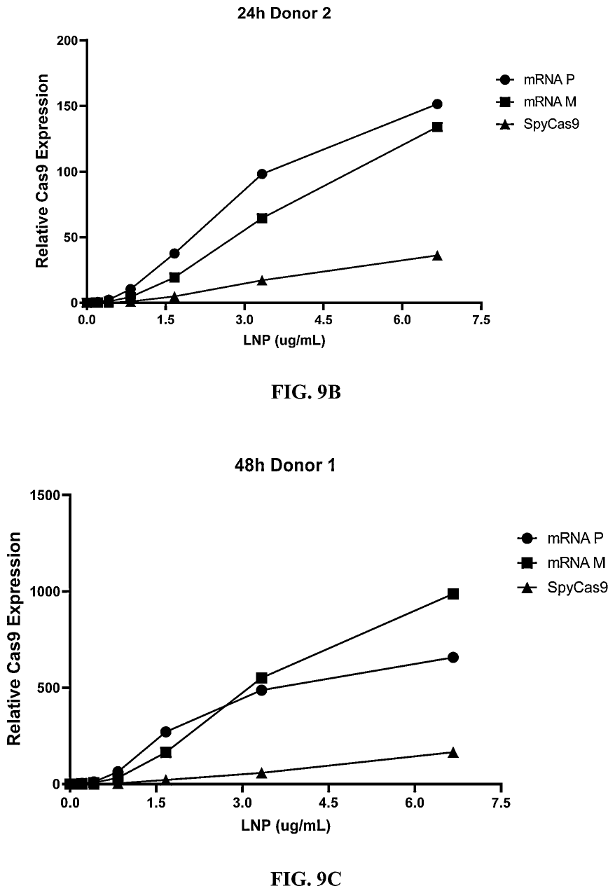

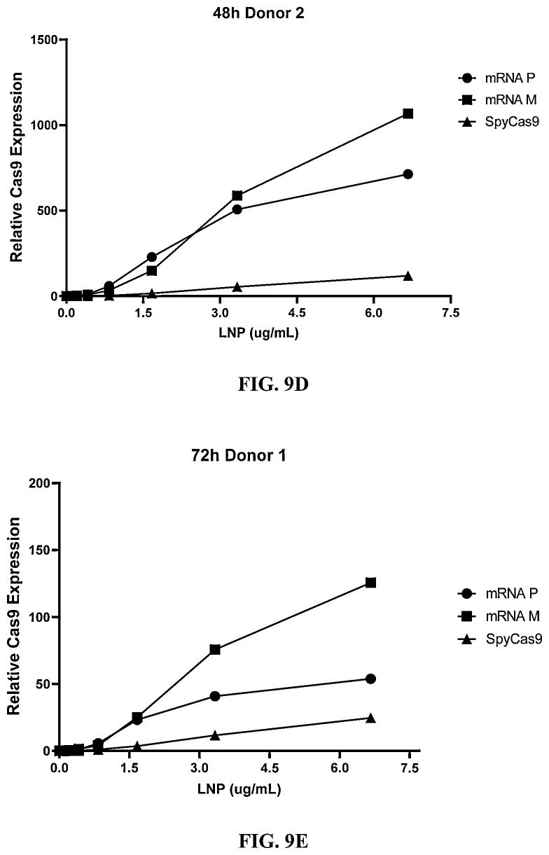

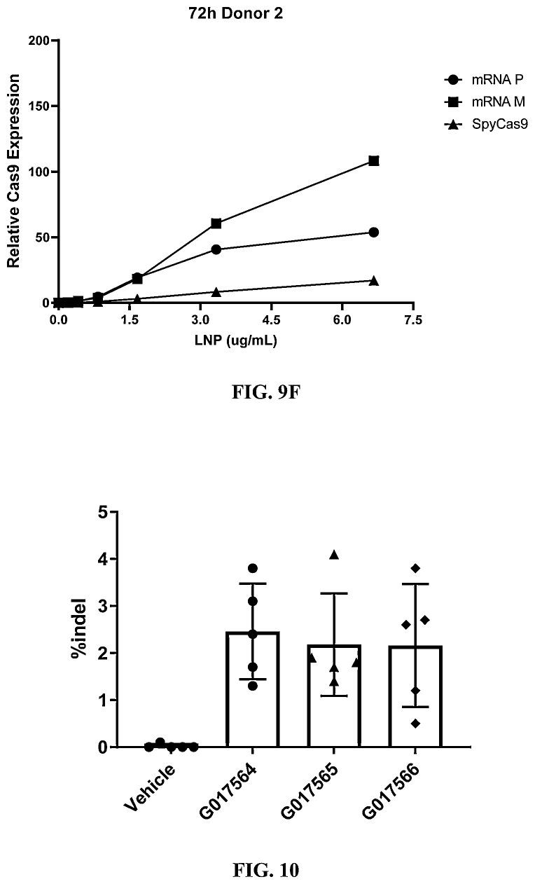

Samples were normalized to the mean of SpyCas9 at 0.83 ug/ml LNP dose. Tables 14A-14B, and FIG. 9A-9F shows the relative Cas9 protein expression in activated cells when mRNA at 24, 48, and 72 hours post LNP treatment in Donor 1 or Donor 2. Cas9 was expressed in a dose dependent manner in activated T cells. Protein expression was higher from Nme2Cas9 samples in comparison to the SpyCas9 sample in activated T cells.

TABLE 14A
Protein expression normalized to the mean
SpyCas9 0.83 ug/ml sample for donor 1

| | | T cell Donor 1 | | | | | |
|---|---|---|---|---|---|---|---|
| | | mRNA P | | mRNA M | | | |
| Timepoint | LNP | SEQ ID NO: 26 | | SEQ ID NO: 23 | | Spy Cas9 | |
| (hours) | (ug/mL) | Mean | N | Mean | N | Mean | N |
| 24 h | 6.67 | 89.3 | 2 | 86.5 | 2 | 26.5 | 2 |
| | 3.33 | 67.7 | 2 | 50.3 | 2 | 11.3 | 2 |
| | 1.67 | 32.4 | 2 | 13.5 | 2 | 4.4 | 2 |
| | 0.83 | 7.3 | 2 | 2.9 | 2 | 1.0 | 2 |
| | 0.42 | 1.6 | 2 | 0.8 | 2 | 0.2 | 2 |
| | 0.21 | 0.4 | 2 | 0.3 | 2 | 0.1 | 2 |
| | 0.10 | 0.2 | 2 | 0.1 | 2 | 0.0 | 2 |
| | 0.00 | 0.0 | 2 | 0.0 | 2 | 0.0 | 2 |
| 48 h | 6.67 | 657.2 | 2 | 987.0 | 2 | 165.9 | 2 |
| | 3.33 | 487.4 | 2 | 551.0 | 2 | 58.5 | 2 |
| | 1.67 | 271.0 | 2 | 165.1 | 2 | 21.4 | 2 |
| | 0.83 | 64.5 | 2 | 32.3 | 2 | 4.3 | 2 |
| | 0.42 | 11.8 | 2 | 7.8 | 2 | 1.0 | 2 |
| | 0.21 | 3.2 | 2 | 2.6 | 2 | 0.1 | 2 |
| | 0.10 | 1.1 | 2 | 0.7 | 2 | 0.1 | 2 |
| | 0.00 | 0.0 | 2 | 0.0 | 2 | 0.0 | 2 |
| 72 h | 6.67 | 53.8 | 2 | 125.6 | 2 | 24.6 | 2 |
| | 3.33 | 40.8 | 2 | 75.6 | 2 | 11.6 | 2 |
| | 1.67 | 23.1 | 2 | 25.0 | 2 | 3.5 | 2 |
| | 0.83 | 5.6 | 2 | 4.0 | 2 | 1.0 | 2 |
| | 0.42 | 1.0 | 2 | 1.3 | 2 | 0.2 | 2 |
| | 0.21 | 0.2 | 2 | 0.4 | 2 | 0.0 | 2 |
| | 0.10 | 0.2 | 2 | 0.0 | 2 | 0.1 | 2 |
| | 0.00 | 0.0 | 2 | 0.0 | 2 | 0.0 | 2 |

TABLE 14B
Protein expression normalized to the mean
SpyCas9 0.83 ug/ml sample for donor 2

| | | T cell Donor 2 | | | | | |
|---|---|---|---|---|---|---|---|
| | LNP | mRNA P | | mRNA M | | SpyCas9 | |
| Timepoint (hours) | (ug/mL) | Mean | SD | Mean | SD | Mean | SD |
| 24 h | 6.67 | 151.5 | 2 | 134.0 | 2 | 36.2 | 2 |
| | 3.33 | 98.2 | 2 | 64.5 | 2 | 17.2 | 2 |
| | 1.67 | 37.6 | 2 | 19.4 | 2 | 4.9 | 2 |
| | 0.83 | 10.4 | 2 | 4.5 | 2 | 1.0 | 2 |
| | 0.42 | 2.4 | 2 | 1.0 | 2 | 0.2 | 2 |
| | 0.21 | 0.7 | 2 | 0.4 | 2 | 0.1 | 2 |
| | 0.10 | 0.3 | 2 | 0.2 | 2 | 0.0 | 2 |
| | 0.00 | 0.0 | 2 | 0.0 | 2 | 0.0 | 2 |
| 48 h | 6.67 | 713.6 | 2 | 1067.3 | 2 | 119.0 | 2 |
| | 3.33 | 507.5 | 2 | 587.1 | 2 | 54.9 | 2 |
| | 1.67 | 229.0 | 2 | 149.2 | 2 | 15.9 | 2 |
| | 0.83 | 59.1 | 2 | 33.8 | 2 | 3.6 | 2 |
| | 0.42 | 10.5 | 2 | 8.3 | 2 | 1.0 | 2 |
| | 0.21 | 3.1 | 2 | 2.8 | 2 | 0.3 | 2 |
| | 0.10 | 1.0 | 2 | 1.9 | 2 | 0.2 | 2 |
| | 0.00 | 0.0 | 2 | 0.2 | 2 | 0.0 | 2 |
| 72 h | 6.67 | 53.8 | 2 | 108.2 | 2 | 17.1 | 2 |
| | 3.33 | 40.7 | 2 | 60.5 | 2 | 8.3 | 2 |
| | 1.67 | 19.3 | 2 | 18.2 | 2 | 3.1 | 2 |
| | 0.83 | 4.7 | 2 | 3.8 | 2 | 1.0 | 2 |
| | 0.42 | 1.3 | 2 | 1.4 | 2 | 0.3 | 2 |
| | 0.21 | 0.4 | 2 | 0.4 | 2 | 0.1 | 2 |
| | 0.10 | 0.3 | 2 | 0.0 | 2 | 0.4 | 2 |
| | 0.00 | 0.0 | 2 | 0.1 | 2 | 0.0 | 2 |

### Example 6. In Vivo Editing in Mouse Liver Using Lipid Nanoparticles (LNPs)

The LNPs used in all in vivo studies were formulated as described in Example 1. Deviations from the protocol are noted in the respective Example. Transport and storage solution (TSS) used in LNP preparation was dosed in the experiment as a vehicle-only negative control.

### In Vivo Editing in the Mouse Model

Selected guide designs were tested for editing efficiency in vivo. CD-1 female mice, ranging 6-10 weeks of age were used in each study involving mice. Animals were weighed pre-dose. LNPs were formulated generally as described in Example 1. LNPs contained a molar ratio of 50% ionizable Lipid A, 38% cholesterol, 9% DSPC, and 3% PEG2k-DMG. The lipid nucleic acid assemblies were formulated with a lipid amine to RNA phosphate (N:P) molar ratio of about 6.

LNPs were dosed via the lateral tail vein at a volume of 0.2 mL per animal (approximately 10 mL per kilogram body weight). Body weight was measured at twenty-four hours post-administration. About 6-7 days after LNP delivery, animals were euthanized by exsanguination under isoflurane anesthesia post-dose. Blood was collected via cardiac puncture into serum separator tubes. For studies involving in vivo editing, liver tissue was collected from the left medial lobe from each animal for DNA extraction and analysis.

For the in vivo studies, genomic DNA was extracted from tissue using a bead-based extraction kit, e.g., the Zymo Quick-DNA 96 kit (Zymo Research, Cat. #D3010) according to the manufacturer's protocol. NGS analysis was performed as described in Example 1.

### Transthyretin (TTR) ELISA Analysis Used in Animal Studies

Blood was collected, and the serum was isolated as described above. The total TTR serum levels were determined using a Mouse Prealbumin (Transthyretin) ELISA Kit (Aviva Systems Biology, Cat. OKIA00111). Kit reagents and standards were prepared according to the manufacturer's protocol. Mouse serum was diluted to a final dilution of 10,000-fold with 1×assay diluent. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to controls, which generally were animals sham-treated with vehicle (TSS) unless otherwise indicated. Percent TSS was calculated by division of each sample TTR value by the average value of the TSS group then adjusted to a percentage value.

### Example 6.1. In Vivo Editing Using Co-Formulated LNPs

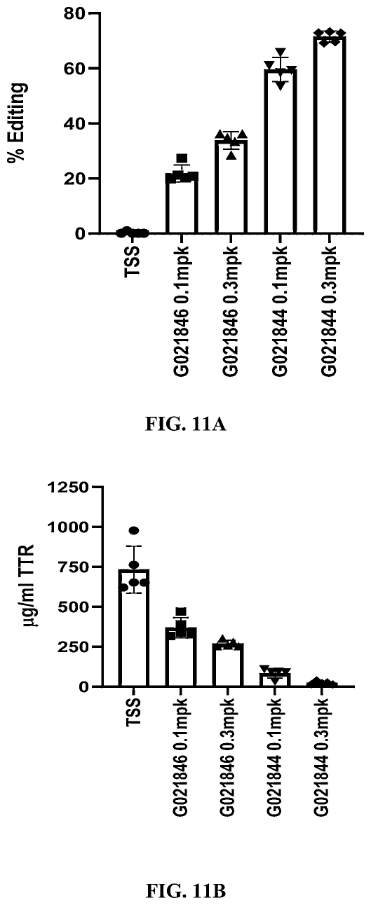

The editing efficiency of the modified sgRNAs tested in Example 4.2 were further evaluated in a mouse model. Guide RNA designs with identical guide sequences targeting mouse PCSK9 but with conserved regions differing lengths were tested LNPs were prepared as described in Example 1. The LNPs were prepared using ionizable lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6. A gRNA targeting the PCSK9 gene, as indicated in Table 15, and mRNA C were co-formulated at 1:2 gRNA to mRNA by weight in LNPs. LNPs were administered to female CD-1 mice (n=5) at a dose of 1 mg/kg of total RNA as described above. Mice were euthanized at 7 days post dosing. The editing efficiency for LNPs containing the indicated sgRNAs are shown in Table 15 and illustrated in FIG. 10.

TABLE 15
Mean percent editing in mouse liver.

| | Dose | Mean | |
|---|---|---|---|
| Guide | (mg/kg) | % Edit | SD |
| Vehicle | — | 0.0 | 0.0 |
| G017564 | 1 | 2.5 | 0.9 |
| G017565 | 1 | 2.2 | 1.0 |
| G017566 | 1 | 2.2 | 1.2 |

### Example 6.2. In Vivo Editing Using pgRNA and mRNA LNPs

The editing efficiency of modified pgRNAs were evaluated in vivo. Four nucleotides in each of the loops of the repeat/anti-repeat region, hairpin 1, and hairpin 2 were substituted with Spacer-18 PEG linkers, in addition to the guide modifications specified in the previous study in Example 6.1.

LNPs were generally prepared as described in Example 1 with a single RNA species as cargo. The LNPs contained lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6.

LNPs containing gRNAs targeting TTR gene indicated in Table 16 were administered to female CD-1 mice (n=5) at a dose of 0.1 mg/kg or 0.3 mg/kg of total RNA as described above. LNP containing mRNA (mRNA M SEQ ID NO: 23) and LNP containing a pgRNA (G021846 or G021844) were delivered simultaneously at a ratio of 1:2 by RNA weight, respectively. Mice were euthanized at 7 days post dose.

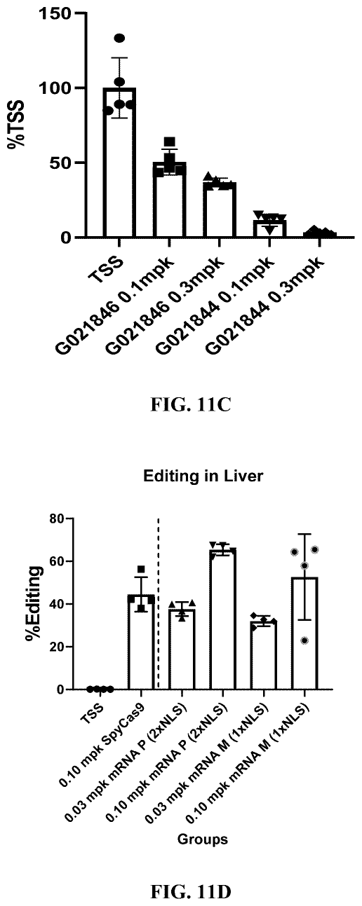

The editing efficiency, serum TTR knockdown, and percent TSS for the LNPs containing the indicated pgRNAs are shown in Table 16 and illustrated in FIG. 11A-11C respectively.

TABLE 16
Liver Editing, Serum TTR protein, and TTR protein knockdown

| | | | | Mean | | | |
|---|---|---|---|---|---|---|---|
| | | | | serum | | | |
| | Dose | Mean | | TTR | | Mean | |
| Guide | (mg/kg) | % Edit | SD | (ug/ml) | SD | % TSS | SD |
| TSS | NA | 0.1 | 0 | 733.1 | 131.2 | 100 | 17.9 |
| G021846 | 0.1 | 21.9 | 2.8 | 369.5 | 56.2 | 50.4 | 7.7 |
| | 0.3 | 33.8 | 2.9 | 269.8 | 21.3 | 36.8 | 2.6 |
| G021844 | 0.1 | 59.6 | 3.9 | 84.1 | 26.6 | 11.5 | 3.6 |
| | 0.3 | 71.6 | 1.8 | 24.4 | 9.2 | 3.3 | 1.2 |

A pgRNA (G021844) from the study described above was evaluated in mice with alternative mRNAs at varied dose levels. LNPs were generally prepared as described in Example 1 with a single RNA species as cargo. LNPs containing pgRNA (G21844) or mRNA (mRNA P or mRNA M) were formulated as described in Example 1. The LNPs used in were prepared with ionizable lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6. Both G000502 and G021844 target exon 3 of the mouse TTR gene. LNP containing pgRNA and LNP containing mRNA were dosed simultaneously based on combined RNA weight at a ratio of 2:1 guide:mRNA by RNA weight, respectively. An additional LNP was co-formulated with G000502 and SpyCas9 mRNA at a ratio of 1:2 by weight, respectively, a preferred SpyCas9 guide:mRNA ratio.

LNPs indicated in Table 17 were administered to female CD-1 mice (n=4) at a dose of 0.1 mg/kg or 0.03 mg/kg of total RNA. The editing efficiency for LNPs containing the indicated gRNAs are shown in Table 17 and illustrated in FIGS. 11D and 11E.

TABLE 17
Liver Editing and Serum TTR protein knockdown

| | | Dose | Mean % | | Mean serum | |
|---|---|---|---|---|---|---|
| Guide | mRNA | (mg/kg) | Edit | SD | TTR (ug/ml) | SD |
| TSS | TSS | NA | 0.12 | 0.04 | 937.4 | 100.5 |
| G000502 | SpyCas9 | 0.1 | 44.50 | 6.9 | 370.7 | 80.1 |
| G021844 | mRNA P | 0.03 | 37.70 | 2.9 | 398.7 | 41.9 |
| | SEQ ID | 0.1 | 65.40 | 2.2 | 92.8 | 27.5 |
| | NO: 26 | | | | | |
| | mRNA M | 0.03 | 32.02 | 2.1 | 527.4 | 93.6 |
| | SEQ ID | | | | | |
| | NO: 23 | 0.1 | 62.50 | 17.4 | 268.6 | 236.8 |

### Example 6.3. In Vivo Editing Using sgRNA and mRNA LNPs

LNPs were generally prepared as described in Example 1 with a single RNA species as cargo. The LNPs used in were prepared with Lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6. The sgRNAs were designed to target the pcsk9 gene (G020361) or the Rosa26 gene (G020848).

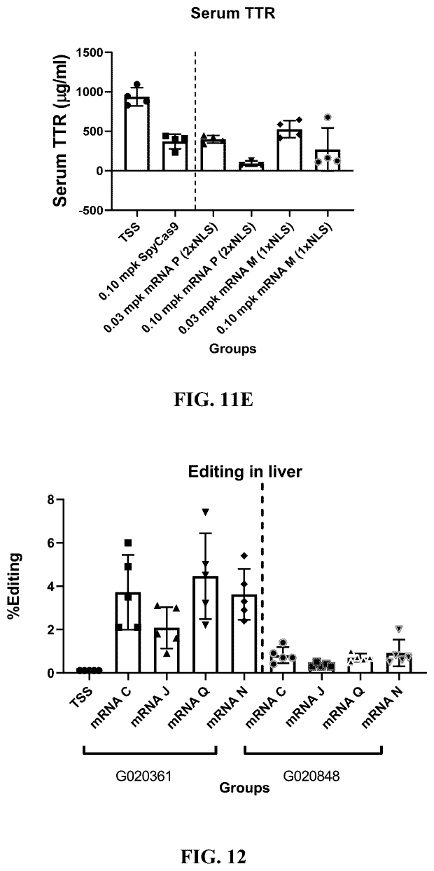

LNPs containing sgRNA or mRNA were administered to female CD-1 mice (n=5) at a dose of 1 mg/kg of total RNA. The mRNAs tested (mRNA C, mRNA J, mRNA Q, mRNA N) were designed with varying numbers and arrangements of NLS. LNPs were dosed simultaneously based on the combined weight of RNA cargo at a 1:1 ratio of gRNA:mRNA by RNA weight. Mean percent editing is shown in Table 18 and illustrated in FIG. 12.

TABLE 18
Mean percent editing in mouse liver.

| | | Dose | Mean | |
|---|---|---|---|---|
| Guide | mRNA | (mg/kg) | % indel | SD |
| TSS | TSS | NA | 0.1 | 0.0 |
| G020361 | mRNA C | 1 | 3.7 | 1.5 |
| | mRNA J | 1 | 2.1 | 0.8 |
| | mRNA Q | 1 | 4.5 | 1.8 |
| | mRNA N | 1 | 3.6 | 1.0 |
| G020848 | mRNA C | 1 | 0.8 | 0.3 |
| | mRNA J | 1 | 0.4 | 0.1 |
| | mRNA Q | 1 | 0.7 | 0.2 |
| | mRNA N | 1 | 0.9 | 0.5 |

### Example 7. In Vivo Editing with NmeCas9 and Either sgRNA or pgRNA

The editing efficiency of the modified pgRNAs tested with Nme2Cas9 was tested in a mouse model. All Nine sgRNAs tested comprised the same 24nt guide sequence targeting mTTR.

LNPs were generally prepared as described in Example 1 with a single RNA species as cargo. The LNPs used in were prepared with Lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs were mixed at a ratio of 2:1 by weight of gRNA to mRNA cargo. Dose is calculated based on the combined RNA mass of gRNA and mRNA. Transport and storage solution (TSS) used in LNP preparation was dosed in the experiment as a vehicle-only negative control.

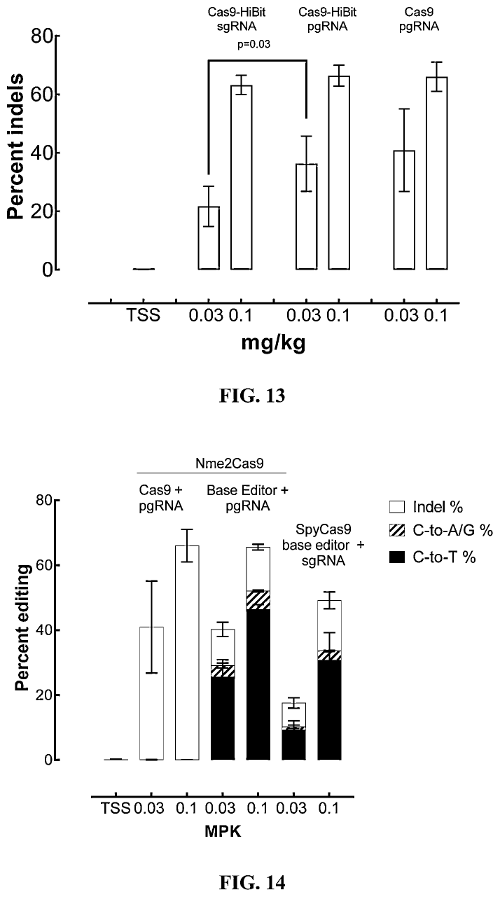

CD-1 female mice, ranging 6-10 weeks of age were used in each study involving mice (n=5 per group, except TSS control n=4). Formulations were administered intravenously via tail vein injection according to the doses listed in Table 19. Animals were periodically observed for adverse effects for at least 24 hours post-dose. Six days after treatment, animals were euthanized by cardiac puncture under isoflurane anesthesia; liver tissue was collected for downstream analysis. Liver punches weighing between 5 and 15 mg were collected for isolation of genomic DNA and total RNA. Genomic DNA samples were analyzed with NGS sequencing as described in Example 1. The editing efficiency for LNPs containing the indicated mRNAs and gRNAs are shown in Table 19 and illustrated in FIG. 13.

TABLE 19
Mean percent editing in mouse liver

| | | Dose | Mean % | | |
|---|---|---|---|---|---|
| mRNA | gRNA | (mg/kg) | Edit | SD | N |
| TSS | TSS | — | 0.08 | 0.05 | 4 |
| mRNA P | G021536 | 0.03 | 21.68 | 6.87 | 5 |
| (2 × N term | (101-nt Nme sgRNA) | 0.1 | 63.22 | 3.28 | 5 |
| NLS, HiBiT) | | | | | |
| mRNA P | G021844 | 0.03 | 36.28 | 9.45 | 5 |
| (2 × N term | (93-nt Nme pgRNA) | 0.1 | 66.44 | 3.55 | 5 |
| NLS, HiBiT) | | | | | |
| mRNA O | G021844 | 0.03 | 40.88 | 14.16 | 5 |
| (2 × N-term | (93-nt Nme pgRNA) | 0.1 | 66.02 | 5.01 | 5 |
| NLS) | | | | | |

### Example 8. In Vivo Base Editing with Nme2Cas9 gRNA

The editing efficiency of the modified gRNAs with different mRNAs were tested with Nine base editor construct in the mouse model. This experiment was performed in parallel to Example 7 and used the same control samples. LNPs were generally prepared as described in Example 1 with a single RNA species as cargo. The LNPs used were prepared with Lipid A, cholesterol, DSPC, and PEG2k-DMG in a molar ratio of 50% Lipid A, 38% cholesterol, 9% DSPC, and 3% PEG2k-DMG. The LNPs were formulated with a lipid amine to RNA phosphate (N:P) molar ratio of about 6. The LNPs used were formulated as described in Example 1, except that each component, guide RNA, or mRNA was formulated individually into an LNP, and the LNP were mixed prior to administration as described in Table 20. For Nme2Cas9 and Nme2Cas9 base editor samples, LNPs were mixed at a ratio of 2:1 by weight of gRNA to editor mRNA cargo. For SpyCas9 base editor samples, LNPs were mixed at a ratio of 1:2 by weight of gRNA to editor mRNA cargo. Dose, as indicated in Table 20 and FIG. 14, is calculated based on the combined RNA weight of gRNA and editor mRNA. Base editor samples were treated with an additional 0.03 mpk of UGI mRNA. Transport and storage solution (TSS) used in LNP preparation was dosed in the experiment as a vehicle-only negative control.

CD-1 female mice, ranging 6-10 weeks of age were used in each study involving mice (n=5 per group, except TSS control n=4). Formulations were administered intravenously via tail vein injection according to the doses listed in Table 20. Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA and total RNA. Genomic DNA was extracted using a DNA isolation kit (ZymoResearch, D3010) and samples were analyzed with NGS sequencing as described in Example 1. The editing efficiency for LNPs containing the indicated gRNAs are shown in Table 20 and illustrated in FIG. 14.

TABLE 20
Mean percent editing in mouse liver.

| | Dose | C-to-T % | | | C-to-A/G % | | | Indel % | | |
|---|---|---|---|---|---|---|---|---|---|---|
| Sample | (mg/kg) | Mean | SD | n | Mean | SD | n | Mean | SD | n |
| TSS | 0 | 0.00 | 0.00 | 4 | 0.10 | 0.00 | 4 | 0.08 | 0.05 | 4 |
| mRNA O + G021844 | 0.03 | 0.00 | 0.00 | 5 | 0.08 | 0.04 | 5 | 40.88 | 14.16 | 5 |
| (Nme2Cas9 + pgRNA) | 0.1 | 0.00 | 0.00 | 5 | 0.02 | 0.04 | 5 | 66.02 | 5.01 | 5 |
| mRNA S + mRNA G + | 0.03 | 25.60 | 5.28 | 5 | 3.50 | 0.76 | 5 | 11.14 | 2.18 | 5 |
| G021844 | 0.1 | 46.34 | 1.53 | 5 | 5.74 | 0.33 | 5 | 13.52 | 0.90 | 5 |
| (Nme2 base editor + UGI + | | | | | | | | | | |
| pgRNA) | | | | | | | | | | |
| mRNA E + mRNA G + | 0.03 | 9.28 | 2.82 | 5 | 0.94 | 0.54 | 5 | 7.34 | 1.61 | 5 |
| G000502 | 0.1 | 30.72 | 8.51 | 5 | 2.86 | 0.23 | 5 | 15.60 | 2.58 | 5 |
| (SpyBC22n + UGI + sgRNA) | | | | | | | | | | |

### Example 9. Guide Screen with Nme1Cas9 and Nme3Cas9 mRNAs in T Cells

The editing efficiency of one modified gRNA scaffold was tested in T cells with Nme1Cas9 or Nme3Cas9 mRNA using guides with 9 distinct target sequences in the TRAC locus.

Healthy human donor apheresis was obtained commercially (Hemacare, Donor 3786), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for use in Cryostor® CS10 (StemCell Technologies Cat. 07930). Upon thawing, T cells were plated at a density of 1.0×10\hat{ }⁶ cells/mL in T cell growth media (TCGM) composed of CTS OpTmizer T Cell Expansion SFM and T Cell Expansion Supplement (Thermo Fisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15). T cells were rested in this media for 24 hours, at which time they were activated with T Cell TransAct™, human reagent (Miltenyi, Cat. 130-111-160) added at a 1:100 ratio by volume.

For Nme1Cas9 guide screening, solutions containing mRNA encoding Nme1Cas9 (mRNA AB) were prepared in P3 buffer. Guide RNAs targeting various sites in the TRAC locus were denatured for 2 minutes at 95° C. and incubated at room temperature for 5 minutes. Forty-eight hours post activation, T cells were harvested, centrifuged, and resuspended at a concentration of 12.5×10\hat{ }⁶ cells/mL in P3 electroporation buffer (Lonza). For each well to be electroporated, 1×10\hat{ }⁵ cells were mixed with 600 ng of Nme1Cas9 mRNA and 5 μM of gRNAs in a final volume of 20 μL of P3 electroporation buffer. This mix was transferred in duplicate to 96-well Nucleofector™ plates and electroporated using the manufacturer's pulse code. Electroporated T cells were immediately rested in CTS OpTmizer T cell growth media without cytokines for 15 minutes before being transferred to new flat-bottom 96-well plates containing an additional CTS OpTmizer T cell growth media supplemented with cytokines. The resulting plates were incubated at 37° C. for 3 days. On day 3 post-electroporation, cells were split 1:2 in 2 U-bottom plates.

On day 7 post-electroporation, the plated T cells were assayed by flow cytometry to determine surface expression of the T cell receptor. Briefly, T cells were incubated with antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628). Cells were subsequently washed, resuspended in cell staining buffer and processed on a Cytoflex flow cytometer (Beckman Coulter). Flow cytometry data was analyzed using the FlowJo software package. T cells were gated based on size, shape, viability, and the expression of CD8 and CD3. Samples were run in duplicate.

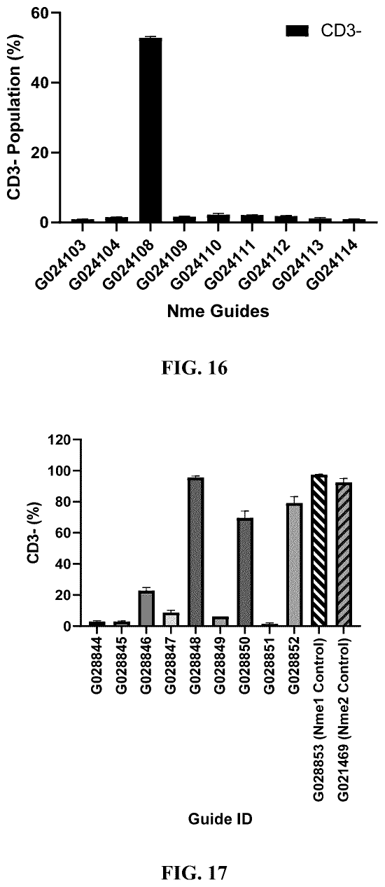

The CD3 is a cell-surface component of the T cell receptor complex and its presence at the cell surface is used as a surrogate marker for TRAC protein expression. CD3 negative cell population, and corresponding standard deviation (SD) for each of the indicated gRNAs are shown in Table 21 and illustrated in FIG. 16.

TABLE 21
Mean percent CD3 negative T cells following
TRAC editing with Nme1Cas9

| Guide ID | Mean | SD |
|---|---|---|
| G024103 | 0.95 | 0.02 |
| G024104 | 1.52 | 0.04 |
| G024108 | 52.82 | 0.40 |
| G024109 | 1.69 | 0.14 |
| G024110 | 2.24 | 0.39 |
| G024111 | 2.10 | 0.06 |
| G024112 | 1.81 | 0.14 |
| G024113 | 1.19 | 0.26 |
| G024114 | 0.97 | 0.05 |

For screening of guides with Nme3Cas9 mRNA, T cells were prepared as described in this example. Solutions containing mRNA encoding Nme3Cas9 (mRNA Z) were prepared in P3 buffer, as well as controls of Nme1Cas9 (mRNA AB) and Nme2Cas9 (mRNA O). Electroporation of an NmeCas9 (e.g., Nme1Cas9, Nme2Cas9, or Nme3Cas9) gRNA and mRNA was performed as described above. Samples were electroporated in triplicate. On day 3 post electroporation, cells were assayed via flow cytometry as described above.

The CD3-negative cell population and corresponding standard deviation (SD) for each of the indicated gRNAs are shown in Table 22 and illustrated in FIG. 17.

TABLE 22
Mean percent CD3 negative T cells following
TRAC editing with Nme3Cas9.

| Guide ID | Mean | SD |
|---|---|---|
| G028844 | 2.99 | 0.49 |
| G028845 | 2.97 | 0.30 |
| G028846 | 22.83 | 1.65 |
| G028847 | 8.71 | 1.16 |
| G028848 | 95.6 | 0.74 |
| G028849 | 6.24 | 0.02 |
| G028850 | 69.63 | 3.57 |
| G028851 | 1.49 | 0.53 |
| G028852 | 79.13 | 3.34 |
| G028853 (Nme1 Control) | 97.43 | 0.20 |
| G021469 (Nme2 Control) | 92.46 | 2.00 |

### Example 10. Expression of Codon Optimized NmeCas9 mRNAs

To quantify expression of each mRNA construct, protein expression levels were measured following electroporation of mRNAs encoding Nme1 Cas9, Nme2Cas9, or Nme3Cas9 into T cells. All of the NmeCas9 mRNA constructs have the same general structure with sequential SV40 and nucleoplasmin nuclear localization signal coding sequences N-terminal to the NmeCas9 open reading frame. Constructs include a coding sequence for a HiBiT tag C-terminal to the NmeCas9 open reading frame. The components are joined by linkers and the specific sequences are provided herein.

Healthy human donor apheresis was obtained commercially (Hemacare, Donor 3786), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930). Upon thawing, T cells were plated at a density of 1.0×10\hat{ }⁶ cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15). T cells were rested in this media for 24 hours, at which time they were activated with T Cell TransAct™, human reagent (Miltenyi, Cat. 130-111-160) added at a 1:100 ratio by volume.

Solutions containing mRNA encoding NmeCas9 were prepared in P3 buffer. Guide RNAs targeting the TRAC locus were removed from the storage and denatured for 2 minutes at 95° C. and incubated at room temperature for 5 minutes. Forty-eight hours post activation, T cells were harvested, centrifuged, and resuspended at a concentration of 12.5×10\hat{ }⁶ cells/mL in P3 electroporation buffer (Lonza). Each well to be electroporated contained 1×10\hat{ }⁵ cells, NmeCas9 mRNA as specified in Table 23, and 1 μM gRNAs (G028853 for Nme1Cas9; G021469 for Nme2Cas9; G028848 for Nme3Cas9) as specified in Table 23 in a final volume of 20 μL of P3 electroporation buffer. NmeCas9 mRNA was tested using a three-fold, five point serial dilution starting at 600 ng mRNA. The appropriate gRNA & mRNA mix was transferred in triplicate to 96-well Nucleofector™ plates and electroporated using the manufacturer's pulse code. Electroporated T cells were immediately rested in CTS OpTmizer T cell growth media without cytokines for 15 minutes before being transferred to new flat-bottom 96-well plates containing an additional CTS OpTmizer T cell growth media supplemented with cytokines. The resulting plates were incubated at 37° C. for 24 hours prior to HiBiT luminescence assay or 96 hours prior to flow cytometry.

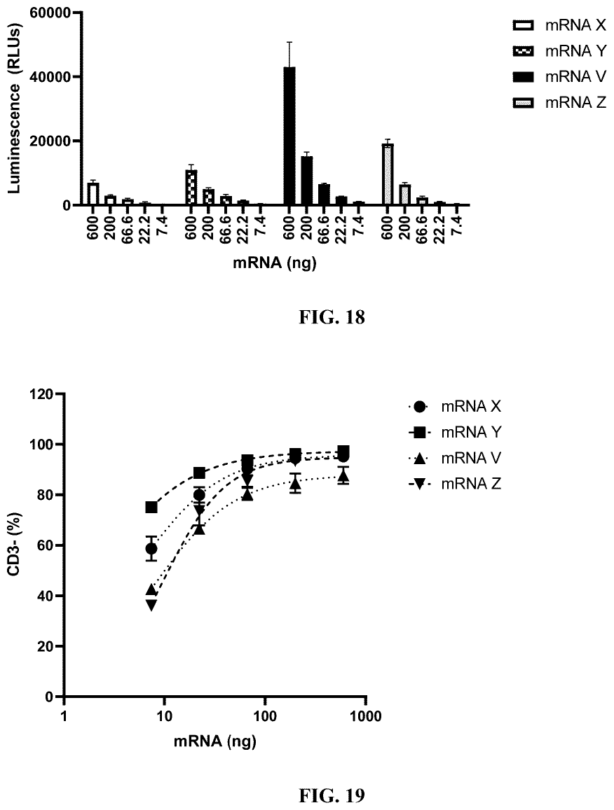

T cells were harvested for protein expression analysis at 24 h post-electroporation. T cells were lysed by Nano-Glo® HiBiT Lytic Assay (Promega). Luminescence was measured using the Biotek Neo2 plate reader. Table 23 and FIG. 18 show the Cas9 protein expression and corresponding standard deviation (SD) in activated cells as relative luminescence units (RLU).

TABLE 23
Mean luminescence (RLU) as a relative measure of
Cas9 protein expression in T cells at 24 hours.

| | mRNA | | | | | |
|---|---|---|---|---|---|---|
| | (ng) | 600 | 200 | 66.6 | 22.2 | 7.4 |
| mRNA X | Mean | 6955.7 | 2941.0 | 1893.7 | 758.3 | 288.7 |
| (Nme1Cas9) | (RLUs) | | | | | |
| G028853 | SD | 800.5 | 232.0 | 268.3 | 256.0 | 21.4 |
| mRNA Y | Mean | 10999.7 | 4967.6 | 2888.7 | 1423.0 | 479.3 |
| (Nme1Cas9) | (RLUs) | | | | | |
| G028853 | SD | 1621.8 | 444.6 | 451.5 | 213.3 | 42.0 |
| mRNA V | Mean | 43026.7 | 15244.0 | 6522.3 | 2658.3 | 1067.3 |
| (Nme2Cas9) | (RLUs) | | | | | |
| G021469 | SD | 7729.3 | 1288.1 | 229.0 | 174.3 | 127.1 |
| mRNA Z | Mean | 19217.3 | 6488.0 | 2386.0 | 1033.3 | 414.7 |
| (Nme3Cas9) | (RLUs) | | | | | |
| G028848 | SD | 1311.8 | 521.0 | 394.3 | 93.6 | 50.2 |

On day 4 post-editing, T cells were assayed by flow cytometry to determine surface protein expression. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100. Cells were subsequently washed, resuspended in 100 μL of cell staining buffer and processed on a Cytoflex flow cytometer (Beckman Coulter). Flow cytometry data were analyzed using the FlowJo software package. T cells were gated based on size, shape, viability, CD8, and CD3. Samples were run in triplicate. The CD3-negative cell population and corresponding standard deviation (SD) for each of the indicated gRNAs are shown in Table 24 and illustrated in FIG. 19.

TABLE 24
Percent CD3-negative cells of T cells following TRAC editing.

| | mRNA (ng) | 600 | 200 | 66.7 | 22.2 | 7.4 |
|---|---|---|---|---|---|---|
| mRNA X | Mean | 95.2 | 94.6 | 90.3 | 79.9 | 58.7 |
| (Nme1Cas9) | SD | 0.8 | 1.1 | 0.6 | 3.1 | 4.8 |
| G028853 | | | | | | |
| mRNA Y | Mean | 97.2 | 96.2 | 93.7 | 88.7 | 75.0 |
| (Nme1Cas9) | SD | 0.8 | 1.1 | 0.9 | 1.4 | 1.3 |
| G028853 | | | | | | |
| mRNA V | Mean | 87.7 | 84.6 | 80.3 | 66.6 | 42.7 |
| (Nme2Cas9) | SD | 3.3 | 3.9 | 2.4 | 1.6 | 0.1 |
| G021469 | | | | | | |
| mRNA Z | Mean | 96.6 | 93.4 | 85.5 | 73.6 | 36.0 |
| (Nme3Cas9) | SD | 0.1 | 1.1 | 2.3 | 5.8 | 2.0 |
| G028848 | | | | | | |

### Example 11. In Vitro Editing with Selected Guides in Primary Cynomolgus Monkey Hepatocytes (PCH)

Three NmeCas9 sgRNAs (G024739, G024741, and G024743) were selected for evaluation in a dose response assay. The tested NmeCas9 sgRNAs targeting the cynomolgus TTR gene include a 24-nucleotide guide sequence.

Unmodified and modified versions of the guides are provided in Table 25.

TABLE 25
Unmodified and modified versions of select gRNAs

| Guide ID | Unmodified sequence | Modified sequence |
|---|---|---|
| G024739 | AGGACCAGCCUCAGACACA | mA*mG*mG*mAmCCAmGmCCmUCmA |
| | AAUACGUUGUAGCUCCCUG | GACAmCAAAmUACmGUUGmUmAmG |
| | AAACCGUUGCUACAAUAAG | mCUCCCmUmGmAmAmAmCmCGUUm |
| | GCCGUCGAAAGAUGUGCCG | GmCUAmCAAU*AAGmGmCCmGmUmC |
| | CAACGCUCUGCCUUCUGGC | mGmAmAmAmGmAmUGUGCmCGmCA |
| | AUCGUU (SEQ ID NO: 509) | AmCGCUCUmGmCCmUmUmCmUGGCA |
| | | UCG*mU*mU (SEQ ID NO: 456) |
| G024741 | CUGCCUCGGACGGCAUCUA | mC*mU*mG*mCmCUCmGmGAmCGmG |
| | GAACUGUUGUAGCUCCCUG | CAUCmUAGAmACUmGUUGmUmAmG |
| | AAACCGUUGCUACAAUAAG | mCUCCCmUmGmAmAmAmCmCGUUm |
| | GCCGUCGAAAGAUGUGCCG | GmCUAmCAAU*AAGmGmCCmGmUmC |
| | CAACGCUCUGCCUUCUGGC | mGmAmAmAmGmAmUGUGCmCGmCA |
| | AUCGUU (SEQ ID NO: 510) | AmCGCUCUmGmCCmUmUmCmUGGCA |
| | | UCG*mU*mU (SEQ ID NO: 457) |
| G024743 | AGGCAGAGGAGGAGCAGA | mA*mG*mG*mCmAGAmGmGAmGGmA |
| | CGAUGAGUUGUAGCUCCCU | GCAGmACGAmUGAmGUUGmUmAmG |
| | GAAACCGUUGCUACAAUAA | mCUCCCmUmGmAmAmAmCmCGUUm |
| | GGCCGUCGAAAGAUGUGCC | GmCUAmCAAU*AAGmGmCCmGmUmC |
| | GCAACGCUCUGCCUUCUGG | mGmAmAmAmGmAmUGUGCmCGmCA |
| | CAUCGUU (SEQ ID NO: 511) | AmCGCUCUmGmCCmUmUmCmUGGCA |
| | | UCG*mU*mU (SEQ ID NO: 458) |

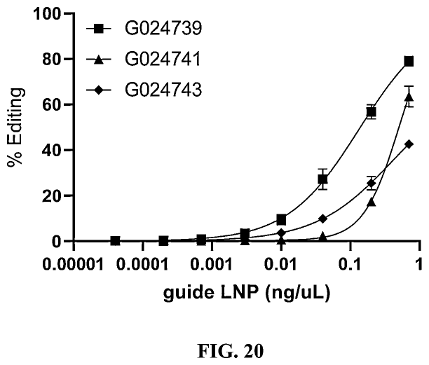

gRNAs and Cas9 mRNA were lipofected, as described below, into primary cynomolgus hepatocytes (PCH). PCH (In Vitro ADMET Laboratories 10136011) were prepared as described in Example 1. PCH were plated at a density of 40,000 cells/well. LNP formulations were prepared as described in Example 1. LNPs were prepared with the lipid composition at a molar ratio of 50% lipid A, 38% cholesterol, 9% DSPC, and 3% PEG2k-DMG. The LNPs were formulated with a lipid amine to RNA phosphate (N:P) molar ratio of about 6 and a gRNA as indicated in Table 25. PCH in 100 μL media were treated with an 8-point, 4-fold dilution series of LNP containing sgRNA, starting at 70 ng, and a fixed 30 ng dose of LNP encapsulating mRNA O by mRNA weight. The sgRNA concentration in each well is indicated in Table 26. The cells were lysed 72 hours post-treatment and NGS analysis was performed as described in Example 1. Dose response of editing efficiency to guide concentration was measured in triplicate samples. Table 26 and FIG. 20 shows mean percent editing and standard deviation (SD) at each guide concentration.

TABLE 26
Mean percent indels at TTR following editing in PCH.

| | | | |
|---|---|---|---|
| Guide LNP | G024739 | G024741 | G024743 |

| (ng/μL) | Mean | SD | Mean | SD | Mean | SD |
|---|---|---|---|---|---|---|
| 0.7 | 79.0 | 1.7 | 63.5 | 3.7 | 42.6 | 1.1 |
| 0.2 | 56.8 | 2.4 | 17.4 | 1.2 | 25.4 | 2.3 |
| 0.04 | 27.2 | 3.6 | 2.3 | 0.5 | 9.8 | 0.5 |
| 0.01 | 9.5 | 1.8 | 0.6 | 0.1 | 3.6 | 0.5 |
| 0.003 | 3.5 | 0.9 | 0.3 | 0.1 | 0.7 | 0.3 |
| 0.0007 | 0.9 | 0.3 | 0.1 | 1.3 | 0.3 | 0.1 |
| 0.0002 | 0.4 | 0.1 | 0.1 | 0.0 | 0.0 | 0.0 |
| 0.00004 | 0.2 | 0.0 | 0.1 | 1.3 | 0.0 | 0.0 |

### Example 12. In Vitro Editing of LNPs Using mRNA Dilution Series in PCH

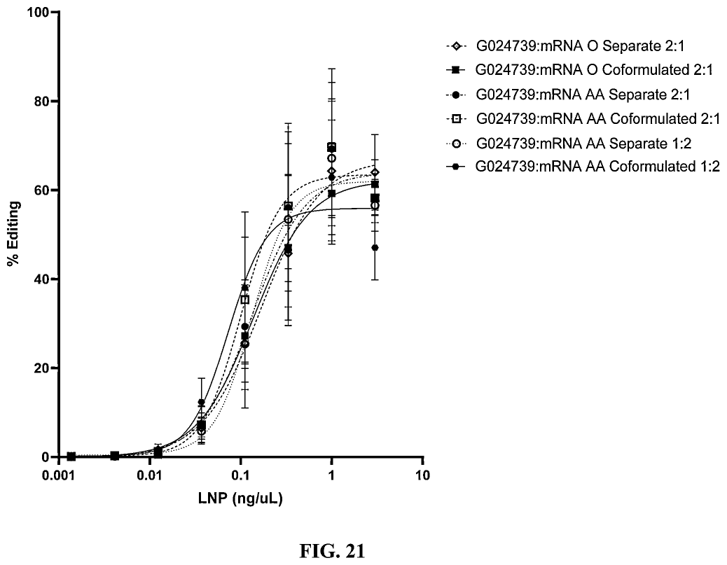

Modified sgRNAs having the same targeting site in the cynomolgus TTR gene were assayed to evaluate the editing efficiency in PCH of different mRNAs (mRNA O, mRNA AA) and formulation ratios. PCH (In Vitro ADMET Laboratories, 10136011) were prepared, treated, and analyzed as described in this example as in Example 1 unless otherwise noted. PCH were used and plated at a density of 50,000 cells/well. LNP formulations were prepared as described in Example 1. LNPs were prepared with a lipid composition having a molar ratio of 50% lipid A, 38% cholesterol, 9% DSPC, and 3% PEG2k-DMG. The LNPs were formulated with a lipid amine to RNA phosphate (N:P) molar ratio of about 6 and a gRNA as indicated in Table 27. PCH in 100 μL media were treated with an 8-point, 3-fold serial dilution of mixed (separately formulated) or co-formulated LNP with various ratios of gRNA:mRNA. The top dose was 3 ng/μL total RNA by weight and gRNA:mRNA ratios for the dilution series were as indicated in Table 27. Samples were run in triplicate. Mean percent editing, standard deviation (SD), and a calculated EC50 are shown in Table 27 and in FIG. 21.

TABLE 27
Mean percent indels at the TTR locus following editing in PCH.

| | | LNP (ng/μL) | | | | | | | | EC50 |
|---|---|---|---|---|---|---|---|---|---|---|
| | | 3 | 1 | 0.33 | 0.11 | 0.04 | 0.01 | 0.004 | 0.001 | (ng/μL) |
| G024739:mRNA | Mean | 64 | 64.3 | 45.7 | 25.6 | 6.5 | 1.6 | 0.2 | 0.1 | 0.17 |
| O LNPs Mixed | % | | | | | | | | | |
| 2:1 | editing | | | | | | | | | |
| | SD | 6.9 | 12.8 | 5.1 | 8.5 | 2.0 | 0.9 | 0.0 | 1.3 | |
| G024739:mRNA | Mean | 58.2 | 69.2 | 46.5 | 29.3 | 7.4 | 0.6 | 0.1 | 0.0 | 0.13 |
| AA LNPs Mixed | % | | | | | | | | | |
| 2:1 | editing | | | | | | | | | |
| | SD | 4.5 | 12.2 | 13.8 | 7.6 | 3.3 | 0.3 | 0.0 | 0.0 | |
| G024739:mRNA | Mean | 56.5 | 67.1 | 53.4 | 25.4 | 5.8 | 0.7 | 0.1 | 0.0 | 0.13 |
| AA LNPs Mixed | % | | | | | | | | | |
| 1:2 | editing | | | | | | | | | |
| | SD | 4.7 | 10.9 | 16.0 | 11.7 | 2.4 | 0.5 | 0.0 | 0.0 | |
| G024739:mRNA | Mean | 61.3 | 59.2 | 47.0 | 27.2 | 7.2 | 0.4 | 0.1 | 0.1 | 0.14 |
| O Coformulated | % | | | | | | | | | |
| 2:1 | editing | | | | | | | | | |
| | SD | 4.4 | 9.3 | 13.2 | 8.4 | 2.2 | 0.2 | 0.0 | 0.0 | |
| G024739:mRNA | Mean | 58.2 | 69.6 | 56.4 | 35.3 | 7.2 | 1.2 | 0.2 | 0.1 | 0.10 |
| AA | % | | | | | | | | | |
| Coformulated | editing | | | | | | | | | |
| 2:1 | SD | 3.0 | 14.4 | 11.4 | 11.4 | 3.3 | 0.4 | 0.1 | 1.3 | |
| G024739:mRNA | Mean | 47.0 | 62.8 | 56.1 | 38 | 12.3 | 1.3 | 0.1 | 0.1 | 0.07 |
| AA | % | | | | | | | | | |
| Coformulated | editing | | | | | | | | | |
| 1:2 | SD | 5.8 | 10.5 | 15.3 | 13.9 | 4.3 | 0.1 | 0.0 | 1.3 | |

### Example 13. In Vivo Editing with NmeCas9 gRNA

The editing efficiency of the modified gRNAs was tested with Nme2Cas9 construct in mice. All Nine sgRNAs tested comprised the same 24 nt guide sequence targeting the mouse TTR gene (mTTR).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6-8 weeks old, were used in each study involving mice. Animals were fed regular chow with standard upkeep. Animals were weighed before dose administration. TSS and LNP formulations were administered intravenously via tail vein injection with a dosage of 0.03 mpk. Animals were periodically observed for adverse effects for at least 24 hours post-dose. Fourteen days after treatment, animals were euthanized by cardiac exsanguination under isoflurane anesthesia; blood for serum preparation and liver tissue were collected for downstream analysis.

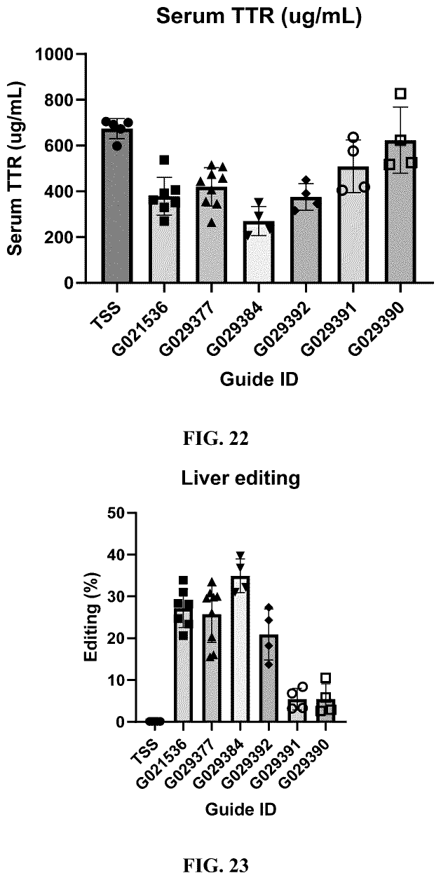

Serum TTR levels shown in Table 28 and FIG. 22 were produced using Serum TTR ELISA—Prealbumin ELISA (Aviva Systems; cat #OKIA00111) according to the manufacturer's protocol for all experimental groups and compared to the negative control (TSS).

TABLE 28
Serum TTR levels (ug/ml).

| | Serum TTR | | | |
|---|---|---|---|---|
| Guide ID | (ug/ml) | SD | % TSS | N |
| TSS | 673.7 | 44.13 | 100 | 5 |
| G021536 | 378.2 | 83.0 | 56.1 | 7 |
| G029377 | 419.3 | 83.5 | 62.2 | 9 |
| G029384 | 270.1 | 63.90 | 40.1 | 4 |
| G029392 | 375.4 | 58.23 | 55.7 | 4 |
| G029391 | 509.1 | 115.3 | 75.6 | 4 |
| G029390 | 623.2 | 144.3 | 92.5 | 4 |

Liver biopsy punches weighing between 5 and 15 mg were collected for isolation of genomic DNA. Genomic DNA was extracted using a DNA isolation kit (ZymoResearch, D3012) and samples were analyzed with NGS sequencing as described in Example 1. The editing efficiency for LNPs containing the indicated gRNAs are shown in Table 29 and illustrated in FIG. 23.

TABLE 29
Mean percent indels at the TTR locus in mouse liver samples

| Guide | Mean % editing | SD | N |
|---|---|---|---|
| TSS | 0.1 | 0 | 5 |
| G021536 | 27.2 | 4.58 | 7 |
| G029377 | 25.7 | 6.77 | 9 |
| G029384 | 34.9 | 4.05 | 4 |
| G029392 | 20.9 | 6.14 | 4 |
| G029391 | 5.4 | 2.60 | 4 |
| G029390 | 5.5 | 3.66 | 4 |

### Example 14. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium followed by centrifugation. The supernatant was discarded and the pelleted cells were resuspended in hepatocyte plating medium (William's E Medium (Gibco, Cat. A12176-01)) with plating supplements dexamethasone+cocktail supplement (Gibco, Cat. A15563, Lot 2459010) and FBS content (Gibco, Cat. A13450, Lot 2486425). Cells were counted and plated on Bio-coat collagen I coated 96-well plates (Corning, Ref 356407, Lot 08722018) at a concentration of 15,000 cells/well. Plated cells were allowed to settle and adhere for 4-6 hours in a tissue culture incubator at 37° C. and 5% CO2 atmosphere. After incubation cells were checked for monolayer formation and were washed once with hepatocyte maintenance medium (William's E Medium) with plating medium supplement (Gibco, Cat. A15564, Lot 2459014).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of 6. Each LNP was applied to cells using an 8 point 3-fold serial dilution starting at 450 ng of total cargo per 100 μl well at the top dose (300 ng mRNA 0 and 46.5 nM gRNA (about 150 ng gRNA))) as shown in Table 30. Upon treatment with LNPs, cells were incubated for 24 hours at 37° C. in William's E Medium with plating medium supplement (Gibco, Cat. A15564, Lot 2459014) and 3% fetal bovine serum. After 72 hours, cells were harvested and analyzed by NGS as described in Example 1.

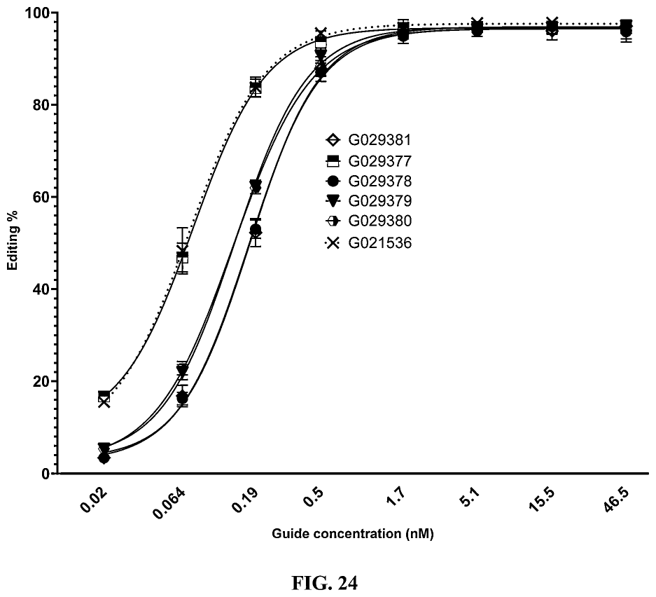

The editing efficiency for LNPs containing the indicated gRNAs, and the corresponding EC50 for each, are shown in Table 30 and illustrated in FIG. 24.

TABLE 30
Mean percent indels at the TTR locus in primary mouse hepatocytes.

| | | | | | | | | | | EC50 |
|---|---|---|---|---|---|---|---|---|---|---|
| | % | nM gRNA | | | | | | | | (nM |
| Guide | indels | 46.5 | 15.5 | 5.1 | 1.7 | 0.5 | 0.19 | 0.064 | 0.02 | gRNA) |
| G021536 | Mean | 97.20 | 97.83 | 97.73 | 96.75 | 95.55 | 83.83 | 48.33 | 15.53 | 0.071 |
| | SD | 0.63 | 0.25 | 0.45 | 1.70 | 0.50 | 2.17 | 4.99 | 0.90 | |
| | N | 4.00 | 4.00 | 4.00 | 4.00 | 4.00 | 4.00 | 4.00 | 4.00 | |
| G029377 | Mean | 96.65 | 96.68 | 96.90 | 96.73 | 93.63 | 83.65 | 46.88 | 16.73 | 0.075 |
| | SD | 0.82 | 1.42 | 1.04 | 0.59 | 2.40 | 1.92 | 3.14 | 1.05 | |
| | N | 4.00 | 4.00 | 4.00 | 4.00 | 4.00 | 4.00 | 4.00 | 4.00 | |
| G029380 | Mean | 95.90 | 97.00 | 96.08 | 95.55 | 88.38 | 62.00 | 22.85 | 5.38 | 0.136 |
| | SD | 2.27 | 0.99 | 1.23 | 1.28 | 2.04 | 0.84 | 1.47 | 1.05 | |
| | N | 4.00 | 4.00 | 4.00 | 4.00 | 4.00 | 4.00 | 4.00 | 4.00 | |
| G029379 | Mean | 97.13 | 96.75 | 96.43 | 95.20 | 90.65 | 62.20 | 22.00 | 5.33 | 0.139 |
| | SD | 0.49 | 1.33 | 1.11 | 1.16 | 1.61 | 1.47 | 1.64 | 0.43 | |
| | N | 4.00 | 4.00 | 4.00 | 4.00 | 4.00 | 4.00 | 4.00 | 4.00 | |
| G029378 | Mean | 95.88 | 97.00 | 96.05 | 94.90 | 87.05 | 53.03 | 16.28 | 3.40 | 0.172 |
| | SD | 1.54 | 0.94 | 1.15 | 1.56 | 2.00 | 2.00 | 1.37 | 0.86 | |
| | N | 4.00 | 4.00 | 4.00 | 4.00 | 4.00 | 4.00 | 4.00 | 4.00 | |
| G029381 | Mean | 97.08 | 96.05 | 96.65 | 95.50 | 87.90 | 52.28 | 16.83 | 3.40 | 0.175 |
| | SD | 0.82 | 1.95 | 0.93 | 0.94 | 1.71 | 3.03 | 2.35 | 0.48 | |
| | N | 4.00 | 4.00 | 4.00 | 4.00 | 4.00 | 4.00 | 4.00 | 4.00 | |

### Example 15. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium followed by centrifugation. The supernatant was discarded and the pelleted cells were resuspended in hepatocyte plating medium (William's E Medium (Gibco, Cat. A12176-01)) with plating supplements dexamethasone+cocktail supplement (Gibco, Cat. A15563, Lot 2459010) and FBS content (Gibco, Cat. A13450, Lot 2486425). Cells were counted and plated on Bio-coat collagen I coated 96-well plates (Corning, Ref 356407, Lot 08722018) at a concentration of 15,000 cells/well. Plated cells were allowed to settle and adhere for 4-6 hours in a tissue culture incubator at 37° C. and 5% CO₂ atmosphere. After incubation cells were checked for monolayer formation and were washed once with hepatocyte maintenance medium (William's E Medium) with plating medium supplement (Gibco, Cat. A15564, Lot 2459014).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of 6. Each LNP was applied to cells using an 8 point 3-fold serial dilution starting at 450 ng of total cargo per 100 μl well at the top dose (300 ng mRNA O and 46.5 nM gRNA (i.e., 150 ng gRNA)) as shown in Table 31. Upon treatment with LNPs, cells were incubated for 24 hours at 37° C. in William's E Medium with plating medium supplement (Gibco, Cat. A15564, Lot 2459014) and 3% fetal bovine serum. Samples were run in triplicates After 72 hours, cells were harvested and analyzed by NGS as described in Example 1.

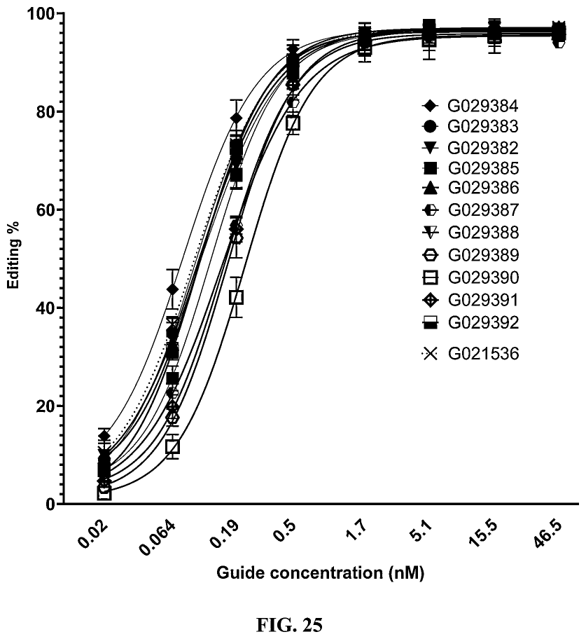

The editing efficiency for LNPs containing the indicated gRNAs, and the corresponding EC50 for each, as shown in Table 31 and illustrated in FIG. 25.

TABLE 31
Mean percent indels at the TTR locus in primary mouse hepatocytes.

| | | | | | | | | | | EC50 |
|---|---|---|---|---|---|---|---|---|---|---|
| | % | nM gRNA | | | | | | | | (nM |
| Guide | indels | 46.5 | 15.5 | 5.1 | 1.7 | 0.5 | 0.19 | 0.064 | 0.02 | gRNA) |
| G029384 | Mean | 96.70 | 97.30 | 96.57 | 95.37 | 92.70 | 78.67 | 43.77 | 13.87 | 0.077 |
| | SD | 0.85 | 0.52 | 1.71 | 1.01 | 1.95 | 3.73 | 4.00 | 1.50 | |
| | N | 3.00 | 3.00 | 3.00 | 3.00 | 3.00 | 3.00 | 3.00 | 3.00 | |
| G021536 | Mean | 97.13 | 96.91 | 96.58 | 95.77 | 90.42 | 73.29 | 36.63 | 10.49 | 0.092 |
| | SD | 0.00 | 0.23 | 0.03 | 0.76 | 1.26 | 1.96 | 1.53 | 0.10 | |
| | N | 18.00 | 18.00 | 18.00 | 18.00 | 18.00 | 18.00 | 18.00 | 18.00 | |
| G029383 | Mean | 96.93 | 96.83 | 96.13 | 94.37 | 90.50 | 73.20 | 34.77 | 9.07 | 0.094 |
| | SD | 0.90 | 1.08 | 2.21 | 2.75 | 3.05 | 2.92 | 2.23 | 0.91 | |
| | N | 3.00 | 3.00 | 3.00 | 3.00 | 3.00 | 3.00 | 3.00 | 3.00 | |
| G029388 | Mean | 96.60 | 97.03 | 96.67 | 95.23 | 89.63 | 71.13 | 34.07 | 9.90 | 0.100 |
| | SD | 0.95 | 0.98 | 2.05 | 2.80 | 0.76 | 2.75 | 3.74 | 3.12 | |
| | N | 3.00 | 3.00 | 3.00 | 3.00 | 3.00 | 3.00 | 3.00 | 3.00 | |
| G029392 | Mean | 96.57 | 95.87 | 96.60 | 95.23 | 90.30 | 72.50 | 31.13 | 6.87 | 0.101 |
| | SD | 1.47 | 2.03 | 1.47 | 2.72 | 3.15 | 1.56 | 1.74 | 0.81 | |
| | N | 3.00 | 3.00 | 3.00 | 3.00 | 3.00 | 3.00 | 3.00 | 3.00 | |
| G029382 | Mean | 96.83 | 97.37 | 96.50 | 95.87 | 89.43 | 69.10 | 33.80 | 9.87 | 0.104 |
| | SD | 0.67 | 0.47 | 1.30 | 1.12 | 3.41 | 4.92 | 4.06 | 0.61 | |
| | N | 3.00 | 3.00 | 3.00 | 3.00 | 3.00 | 3.00 | 3.00 | 3.00 | |
| G029386 | Mean | 97.17 | 97.20 | 96.23 | 95.80 | 91.70 | 71.53 | 32.57 | 9.43 | 0.105 |
| | SD | 0.55 | 1.13 | 0.38 | 1.32 | 0.95 | 0.61 | 1.62 | 0.93 | |
| | N | 3.00 | 3.00 | 3.00 | 3.00 | 3.00 | 3.00 | 3.00 | 3.00 | |
| G029385 | Mean | 96.80 | 95.90 | 96.67 | 94.87 | 87.83 | 66.95 | 25.60 | 7.20 | 0.123 |
| | SD | 0.80 | 0.14 | 1.23 | 1.53 | 3.11 | 2.47 | 2.52 | 0.87 | |
| | N | 3.00 | 3.00 | 3.00 | 3.00 | 3.00 | 3.00 | 3.00 | 3.00 | |
| G029387 | Mean | 94.07 | 95.65 | 95.23 | 94.00 | 81.90 | 56.83 | 22.67 | 5.97 | 0.149 |
| | SD | 0.15 | 0.35 | 0.71 | 0.56 | 2.69 | 1.80 | 0.47 | 0.49 | |
| | N | 3.00 | 3.00 | 3.00 | 3.00 | 3.00 | 3.00 | 3.00 | 3.00 | |
| G029391 | Mean | 95.70 | 95.77 | 95.43 | 94.30 | 85.40 | 56.03 | 19.83 | 4.70 | 0.156 |
| | SD | 1.40 | 2.49 | 2.98 | 2.96 | 3.03 | 2.62 | 2.40 | 0.69 | |
| | N | 3.00 | 3.00 | 3.00 | 3.00 | 3.00 | 3.00 | 3.00 | 3.00 | |
| G029389 | Mean | 96.80 | 96.40 | 95.87 | 94.60 | 85.53 | 54.23 | 17.63 | 3.40 | 0.164 |
| | SD | 1.14 | 0.99 | 2.32 | 1.93 | 2.16 | 4.05 | 1.75 | 0.62 | |
| | N | 3.00 | 3.00 | 3.00 | 3.00 | 3.00 | 3.00 | 3.00 | 3.00 | |
| G029390 | Mean | 96.00 | 95.40 | 94.60 | 93.07 | 77.60 | 42.13 | 11.70 | 2.20 | 0.219 |
| | SD | 1.49 | 3.49 | 3.97 | 2.97 | 2.29 | 4.10 | 2.49 | 0.70 | |
| | N | 3.00 | 3.00 | 3.00 | 3.00 | 3.00 | 3.00 | 3.00 | 3.00 | |

### Example 16. In Vitro Editing in Primary Mouse Hepatocytes (PMH) with Dilution Curve

### A. Example 16.1. Modified sgRNA Evaluation Using Dilution Series

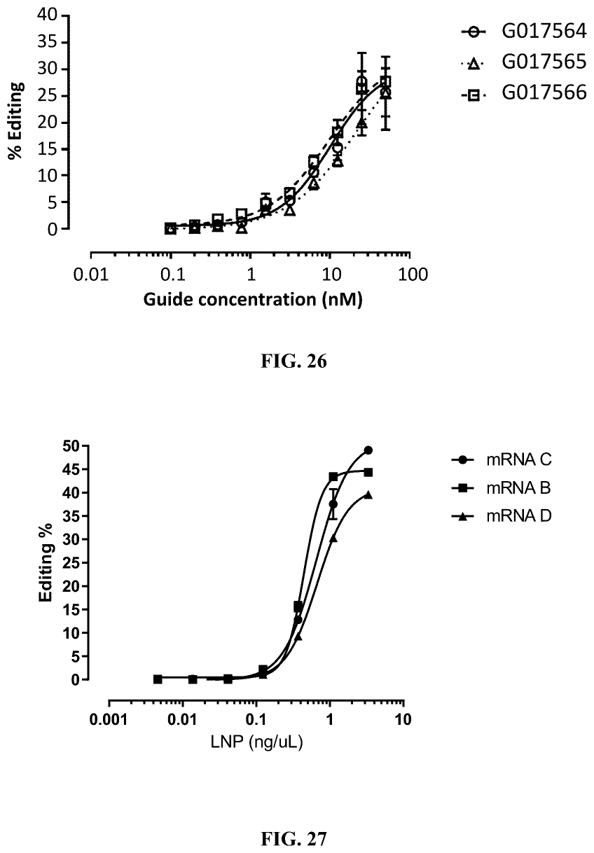

Modified sgRNAs with various scaffold structures, all targeting a previously published site in the mouse pcsk9 gene (see WO2019094791) were designed as shown in Tables 1-2 and tested for editing efficiency using in primary mouse hepatocytes (PMH). Cells were prepared as described in Example 1 using PMH cells (In Vitro ADMET Laboratories) and plated at a density of 20,000 cells/well. Cells were transfected using MessengerMax (Invitrogen) according to the manufacturer's protocols with 1 ng/ul Nme2 Cas9 mRNA (mRNA U) and sgRNA at concentrations as indicated in Table 32. Duplicate samples were included in the assay. Cells were harvested 72 hours following transfection and analyzed by NGS as described in Example 1. Mean percent editing with standard deviation are shown in Table 32 and FIG. 26.

TABLE 32
Mean percent editing in PMH

| Guide | G017564 | | G017565 | | G017566 | |
|---|---|---|---|---|---|---|
| concentration | Mean % | | Mean % | | Mean % | |
| (nM) | editing | SD | editing | SD | editing | SD |
| 50.0 | 25.7 | 3.2 | 25.6 | 4.9 | 27.7 | 0.4 |
| 25.0 | 27.8 | 1.4 | 20.0 | 1.7 | 26.4 | 4.7 |
| 12.5 | 15.3 | 1.7 | 12.9 | 0.8 | 18.2 | 1.7 |
| 6.3 | 10.6 | 0.6 | 8.7 | 0.9 | 12.6 | 0.9 |
| 3.1 | 5.3 | 0.2 | 3.6 | 0.3 | 6.7 | 0.7 |
| 1.6 | 5.0 | 1.1 | 3.6 | 0.6 | 4.8 | 0.2 |
| 0.8 | 1.3 | 0.5 | 0.2 | 0.1 | 2.8 | 0.3 |
| 0.4 | 0.8 | 0.3 | 0.5 | 0.1 | 1.8 | 0.1 |
| 0.2 | 0.3 | 0.1 | 0.2 | 0.1 | 0.7 | 0.1 |
| 0.1 | 0.1 | 0.0 | 0.1 | 0.1 | 0.2 | 0.1 |
| 0.0 | 0.1 | 0.0 | 0.0 | 0.0 | 0.3 | 0.1 |

### B. Example 16.2. Evaluation of mRNA Poly-A Tail Modifications and Cargo Ratios

An sgRNA targeting the mouse psck9 gene was selected from Table 32 to evaluate guide editing efficiency resulting from particular combinations of poly-A tail modifications and sgRNA:mRNA ratios. PMH cells used were prepared, treated, and analyzed as described in Example 1 unless otherwise noted. PMH (Gibco) were plated at a density of 15,000 cells/well.

LNPs were generally prepared as described in Example 1. LNPs were prepared with the lipid composition of 50/9/38/3, expressed as the molar ratio of ionizable lipid A/cholesterol/DSPC/PEG, respectively. The LNPs were formulated with a lipid amine to RNA phosphate (N:P) molar ratio of about 6. LNPs encapsulated gRNA G017566 or one of three mRNAs encoding the same Nme2Cas9 open reading frame (ORF) but with different encoded poly-A tails, as indicated in Table 33. A preliminary experiment holding the sgRNA application constant and varying the amount of mRNA applied showed that 1:1 sgRNA:mRNA ratio by weight resulted in the highest percent editing. In the current Example, increasing doses mRNA LNP and gRNA LNP were applied to cells in 100 ul media as described in Table 33, maintaining a 1:1 sgRNA:mRNA ratio by weight. Table 33 and FIG. 27 show mean percent editing and standard deviation (SD).

TABLE 33
Mean percent editing in PMH

| mRNA C | mRNA B | mRNA D |
|---|---|---|
| SEQ ID NO: | SEQ ID | SEQ ID NO: |
| 622 | NO: 621 | 623 |

| Total | Mean | | Mean | | Mean | |
|---|---|---|---|---|---|---|
| RNA | % | | % | | % | |
| (ng) | editing | SD | editing | SD | editing | N |
| 333. | 49.1 | 0.9 | 44.3 | 0.0 | 39.6 | 1 |
| 111. | 37.5 | 3.2 | 43.4 | 0.2 | 30.3 | 1 |
| 37. | 12.8 | 0.2 | 15.6 | 1.0 | 9.3 | 1 |
| 12.3 | 1.3 | 0.2 | 2.2 | 0.0 | 1.1 | 1 |
| 4.1 | 0.1 | 0.0 | 0.2 | 0.0 | 0.0 | 1 |
| 1.4 | 0.1 | 0.0 | 0.0 | 0.0 | 0.1 | 1 |
| 0.5 | 0.1 | 0.0 | 0.1 | 0.1 | 0.0 | 1 |

### Example 17. Dose Response Curve for NmeCas9 gRNA in PMH with Nme2Cas9

The editing efficiency of the modified gRNAs was tested with Nme2Cas9 construct in primary mouse hepatocytes (PMH). All Nine sgRNAs tested comprised the same 24nt guide sequence targeting the mouse TTR gene (mTTR).

PMH (Gibco, Lot MC931) were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells resuspended in hepatocyte plating medium plus supplement pack (Invitrogen, Cat. A1217601 and Gibco, Cat. CM3000). Cells were counted and plated on Bio-coat collagen I coated 96-well plates (Thermo Fisher, Cat. 877272) at a concentration of 15,000 cells/well. Plated cells were allowed to settle and adhere for 4-6 hours in a tissue culture incubator at 37° C. and 5% C02 atmosphere. After incubation cells were checked for monolayer formation and were washed once with hepatocyte maintenance medium (Invitrogen, Cat. A1217601 and Gibco, Cat. CM4000).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of 6. Each LNP was applied to cells using an 8 point 4-fold serial dilution starting at 300 ng of total RNA per 100 ul well (about 32.25 nM gRNA concentration per well) as shown in Table 32. Upon treatment with LNPs, cells were incubated for 24 hours at 37° C. in Williams' E Medium (Gibco, A1217601) with maintenance supplements and 3% fetal bovine serum. Samples were run in triplicate. After 72 hours, cells were harvested and analyzed by NGS as described in Example 1.

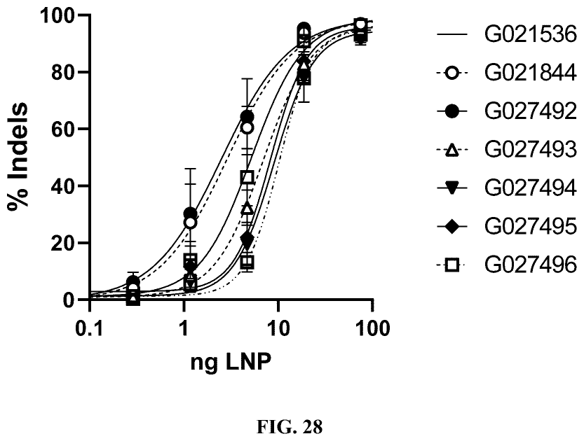

The editing efficiency for LNPs containing the indicated gRNAs, and the corresponding EC50 for each, are shown in Table 34 and illustrated in FIG. 28.

TABLE 34
Mean percent indels at the TTR locus in primary mouse hepatocytes.

| | | | | | | | | | | EC50 |
|---|---|---|---|---|---|---|---|---|---|---|
| | % | ng RNA | | | | | | | | (ng |
| Guide | indels | 300 | 75 | 18.75 | 4.68 | 1.17 | 0.29 | 0.07 | 0 | RNA) |
| G021536 | Mean | 97.7 | 96.4 | 90.9 | 43.1 | 13.9 | 1.3 | 0.3 | 0.0 | 5.23 |
| | SD | 0.5 | 0.3 | 5.0 | 10.1 | 6.5 | 0.7 | 0.1 | 0.0 | |
| G021844 | Mean | 96.7 | 96.9 | 93.8 | 60.6 | 27.2 | 4.0 | 0.5 | 0.3 | 2.86 |
| | SD | 0.8 | 0.3 | 1.9 | 7.5 | 13.4 | 2.7 | 0.3 | 0.1 | |
| G027492 | Mean | 97.1 | 96.5 | 95.2 | 64.3 | 30.2 | 6.2 | 0.5 | 0.0 | 2.49 |
| | SD | 1.4 | 0.4 | 1.6 | 13.4 | 15.9 | 3.5 | 0.4 | 0.1 | |
| G027493 | Mean | 96.5 | 94.9 | 82.7 | 32.4 | 8.6 | 0.9 | 0.0 | 0.0 | 6.95 |
| | SD | 0.1 | 0.6 | 6.4 | 6.2 | 5.5 | 0.8 | 0.1 | 0.0 | |
| G027494 | Mean | 96.0 | 91.7 | 78.3 | 19.6 | 6.7 | 0.7 | 0.0 | 0.0 | 9.06 |
| | SD | 1.0 | 2.2 | 8.9 | 7.6 | 4.0 | 0.4 | 0.1 | 0.0 | |
| G027495 | Mean | 96.0 | 94.6 | 83.8 | 22.0 | 11.6 | 1.8 | 0.2 | 0.1 | 8.31 |
| | SD | 0.5 | 1.8 | 6.8 | 9.5 | 7.3 | 1.8 | 0.1 | 0.1 | |
| G027496 | Mean | 96.2 | 93.2 | 77.8 | 13.2 | 5.4 | 0.4 | 0.1 | 0.0 | 10.22 |
| | SD | 0.7 | 2.9 | 8.4 | 3.4 | 2.7 | 0.4 | 0.1 | 0.0 | |

### Example 18. In Vivo Editing with NmeCas9 gRNA

The editing efficiency of the modified gRNAs was tested with Nme2Cas9 construct in mice. All Nine sgRNAs tested comprised the same 24 nt guide sequence targeting the mouse TTR gene (mTTR).

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6-8 weeks old, were used in each study involving mice (n=5 for all groups). Animals were fed regular chow with standard upkeep. Animals were weighed before dose administration. TSS and LNP formulations were administered intravenously via tail vein injection with a dosage of 0.03 mpk. Animals were periodically observed for adverse effects for at least 24 hours post-dose. Seven days after treatment, animals were euthanized by cardiac exsanguination under isoflurane anesthesia; blood for serum preparation and liver tissue were collected for downstream analysis.

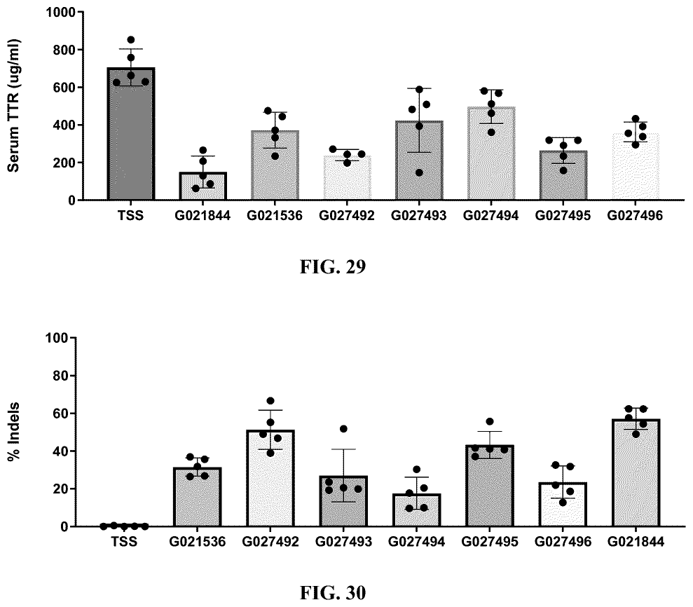

Serum TTR levels shown in Table 35 and FIG. 29 were produced using Serum TTR ELISA—Prealbumin ELISA (Aviva Systems; cat #OKIA00111) according to the manufacturer's protocol. The level of serum TTR is significantly lower in all experimental groups compared to the negative control (TSS).

TABLE 35
Serum TTR levels (ug/ml).

| | Serum TTR | | |
|---|---|---|---|
| Guide ID | (ug/ml) | SD | % TSS |
| TSS | 704.9 | 98.3 | 100% |
| G021844 | 150.0 | 84.9 | 21% |
| G021536 | 371.1 | 95.6 | 53% |
| G027492 | 239.4 | 30.5 | 34% |
| G027493 | 423.4 | 170.0 | 60% |
| G027494 | 496.3 | 89.8 | 70% |
| G027495 | 263.6 | 68.9 | 37% |
| G027496 | 362.4 | 52.7 | 51% |

Liver biopsy punches weighing between 5 and 15 mg were collected for isolation of genomic DNA. Genomic DNA was extracted using a DNA isolation kit (ZymoResearch, D3012) and samples were analyzed with NGS sequencing (n=5 for all groups) as described in Example 1. The editing efficiency for LNPs containing the indicated gRNAs are shown in Table 36 and illustrated in FIG. 30.

TABLE 36
Mean percent indels at the TTR locus in mouse liver samples

| Guide | Mean | SD |
|---|---|---|
| TSS | 0.12 | 0.22 |
| G021844 | 57.1 | 5.7 |
| G021536 | 31.5 | 4.9 |
| G027492 | 51.3 | 10.4 |
| G027493 | 27.0 | 14.0 |
| G027494 | 17.6 | 8.6 |
| G027495 | 43.2 | 7.2 |
| G027496 | 23.5 | 8.6 |

### Example 19. In Vivo Editing with NmeCas9 gRNA

The editing efficiency of the modified gRNAs was tested with Nme2Cas9 mRNA in mice. All Nine sgRNAs tested comprised the same 24nt guide sequence targeting mTTR.

LNPs were generally prepared as described in Example 1 with a cargo of 1:2 by weight of gRNA to mRNA O. The LNPs used were prepared with a molar ratio of 50% Lipid A, 38% cholesterol, 9% DSPC, and 3% PEG2k-DMG. The LNPs were formulated with a lipid amine to RNA phosphate (N:P) molar ratio of about 6. Dose was calculated based on the combined RNA weight of gRNA and mRNA. Transport and storage solution (TSS) used in LNP preparation was dosed in the experiment as a vehicle-only negative control.

CD-1 female mice, about 6 weeks old, were used in each study involving mice (n=5 for all groups). Animals were weighed before dose administration for dose calculation, and 24 hours post-administration for monitoring. TSS and LNP formulations were administered intravenously via tail vein injection with a dosage of 0.01 mpk or 0.03 mpk. Animals were periodically observed for adverse effects for at least 24 hours post-dose. Seven days after treatment, animals were euthanized by cardiac exsanguination under isoflurane anesthesia. Blood was collected by cardiac puncture for Serum TTR ELISA, and liver tissue was collected for downstream analysis.

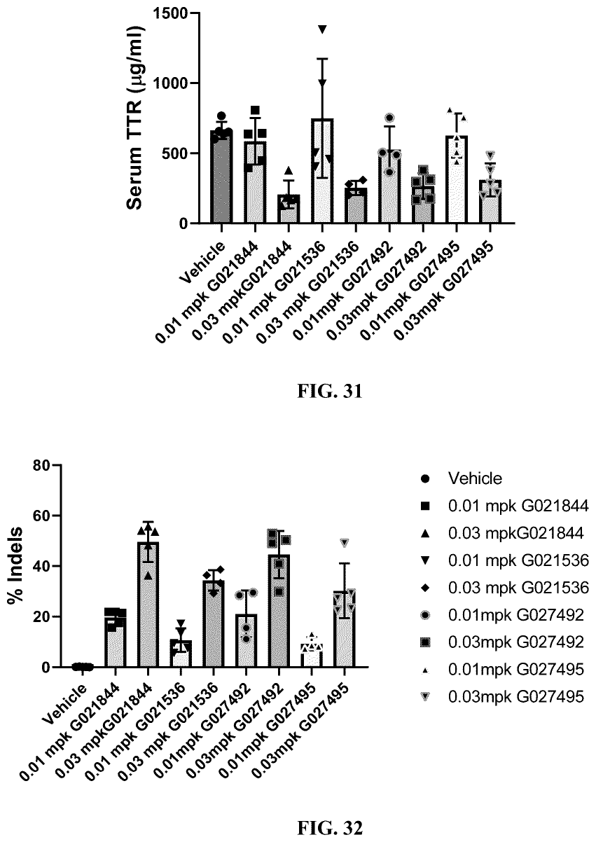

Serum TTR results prepared using Serum TTR ELISA—Prealbumin ELISA (Aviva Systems; cat #OKIA00111) according to the manufacturer's protocol are shown in FIG. 31 and Table 37.

TABLE 37
Serum TTR measurements following treatment.

| Guide ID | Dosage (mpk) | Serum TTR (ug/ml) | SD | N |
|---|---|---|---|---|
| Vehicle | | 663.5 | 61.5 | 5 |
| G021844 | 0.01 | 585.5 | 166.1 | 5 |
| | 0.03 | 205.8 | 99.2 | 5 |
| G021536 | 0.01 | 749.2 | 425.3 | 5 |
| | 0.03 | 252.6 | 50.2 | 4 |
| G027492 | 0.01 | 527.4 | 163.1 | 4 |
| | 0.03 | 266.0 | 92.4 | 5 |
| G027495 | 0.01 | 626.9 | 157.7 | 5 |
| | 0.03 | 310.0 | 118.1 | 5 |

Liver biopsy punches weighing about 5 mg-15 mg were collected for isolation of genomic DNA and total RNA. Genomic DNA was extracted using a DNA isolation kit (ZymoResearch, D3012) and samples were analyzed with NGS sequencing (n=5 for all groups) as described in Example 1. The editing efficiency for LNPs containing the indicated gRNAs are shown in Table 38 and illustrated in FIG. 32.

TABLE 38
Mean percent indels at the TTR locus in mouse liver samples.

| Guide ID | Dosage | Mean | SD | N |
|---|---|---|---|---|
| Vehicle | 0.00 | 0.1 | 0.07 | 5 |
| G021844 | 0.01 | 19.7 | 2.9 | 5 |
| | 0.03 | 49.6 | 7.9 | 5 |
| G021536 | 0.01 | 10.7 | 4.7 | 5 |
| | 0.03 | 34.4 | 4.1 | 4 |
| G027492 | 0.01 | 21.1 | 9.2 | 4 |
| | 0.03 | 44.6 | 9.4 | 5 |
| G027495 | 0.01 | 9.3 | 2.6 | 4 |
| | 0.03 | 30.2 | 10.9 | 5 |

### Example 20. Additional Embodiments

The following numbered items provide additional support for and descriptions of the embodiments herein.

Item 1 is a polynucleotide comprising an open reading frame (ORF), the ORF comprising: a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321, wherein the Nine Cas9 is an Nme2 Cas9, an Nme1 Cas9, or Nme3 Cas9; and a nucleotide sequence encoding a first nuclear localization signal (NLS).

Item 2 is the polynucleotide of Item 1, wherein the ORF further comprises a nucleotide sequence encoding a second NLS.

Item 3 is the polynucleotide of Item 1, wherein the first and second NLS are independently selected from SEQ ID NO: 388 and 410-422.

Item 4 is the polynucleotide of any one of the preceding Items, wherein the polynucleotide further comprises a polyA sequence or a polyadenylation signal sequence.

Item 5 is the polynucleotide of Item 4, wherein the polyA sequence comprises non-adenine nucleotides.

Item 6 is the polynucleotide of Item of any one of Items 4-5, wherein the polyA sequence comprises 100-400 nucleotides.

Item 7 is the polynucleotide of Item of any one of Items 4-6, wherein the polyA sequence comprises a sequence of SEQ ID NO: 409.

Item 8 is the polynucleotide of any one of the preceding Items, wherein the ORF further comprises a nucleotide sequence encoding a linker sequence between the first NLS and the second NLS.

Item 9 is the polynucleotide of any one of the preceding Items, wherein the ORF further comprises a nucleotide sequence encoding a linker spacer sequence between the Nine Cas9 coding sequence and the NLS proximal to the Nine Cas9 coding sequence.

Item 10 is the polynucleotide of Item of any one of Items 8-9, wherein the linker comprises at least 1, at least 2, at least 3, at least 4, at least 5, at least 6, at least 7, at least 8, at least 9, or at least 10 amino acids.

Item 11 is the polynucleotide of Item of any one of Items 8-10, wherein the linker sequence comprises GGG or GGGS, optionally wherein the GGG or GGGS sequence is at the N-terminus of the spacer sequence.

Item 12 is the polynucleotide of Item of any one of Items 8-11, wherein the linker sequence comprises a sequence of any one of SEQ ID NOs: 61-122.

Item 13 is the polynucleotide of any one of the preceding Items, wherein the ORF further comprises one or more additional heterologous functional domains.

Item 14 is the polynucleotide of any one of the preceding Items, wherein the Nine Cas9 has double stranded endonuclease activity.

Item 15 is the polynucleotide of any one of Items 1-14, wherein the Nine Cas9 has nickase activity.

Item 16 is the polynucleotide of any one of Items 1-14, wherein the Nine Cas9 comprises a dCas9 DNA binding domain.

Item 17 is the polynucleotide of any one of the preceding Items, wherein the NmeCas9 comprises an amino acid sequence with at least 91%, 92%, 93%, 94%, 95%, 96%, 97%, 98%, or 99% to any one of SEQ ID NOs: 1 and 4-13, 220, 227, 234, 241, 248, 255, 262, 269, 276, 283, 290, 297, or 310-315.

Item 18 is the polynucleotide of any one of the preceding Items wherein the NmeCas9 comprises an amino acid sequence of SEQ ID NO: 1 and 4-13, 220, 227, 234, 241, 248, 255, 262, 269, 276, 283, 290, 297, or 310-315.

Item 19 is the polynucleotide of any one of the preceding Items, wherein the sequence encoding the NmeCas9 comprises a nucleotide sequence having at least 91%, 92%, 93%, 94%, 95%, 96%, 97%, 98%, or 99% identity to the nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

Item 20 is the polynucleotide of any one of the preceding Items, wherein the sequence encoding the NmeCas9 comprises a nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

Item 21 is a polynucleotide comprising an open reading frame (ORF) encoding a polypeptide comprising: a cytidine deaminase, which is optionally an APOBEC3A deaminase; a nucleotide sequence encoding a C-terminal N. meningitidis (Nine) Cas9 nickase polypeptide at least 90% identical to any one of SEQ ID NOs: 1 and 4-13, 220, 227, 234, 241, 248, 255, 262, 269, 276, 283, 290, or 297, wherein the Nine Cas9 nickase is an Nme2 Cas9 nickase, an Nme1 Cas9 nickase, or an Nme3 Cas9 nickase; and a nucleotide sequence encoding a first nuclear localization signal (NLS); wherein the polypeptide does not comprise a uracil glycosylase inhibitor (UGI).

Item 22 is the polynucleotide of Item 21, wherein the ORF further comprises a nucleotide sequence encoding a second NLS.

Item 23 is the polynucleotide of any one of Items 21-22, wherein the deaminase is located N-terminal to an NLS in the polypeptide.

Item 24 is the polynucleotide of any one of Items 21-23, wherein the cytidine deaminase is located N-terminal to the first NLS and the second NLS in the polypeptide.

Item 25 is the polynucleotide of any one of Items 21-22, wherein the cytidine deaminase is located C-terminal to an NLS in the polypeptide.

Item 26 is the polynucleotide of any one of Items 23-25, wherein the cytidine deaminase is located C-terminal to the first NLS and the second NLS in the polypeptide.

Item 27 is the polynucleotide of any one of Items 21-26, wherein the ORF does not comprise a coding sequence for an NLS C-terminal to the ORF encoding the Nine Cas9.

Item 28 is the polynucleotide of any one of Items 21-26, wherein the ORF does not comprise a coding sequence C-terminal to the ORF encoding the Nine Cas9.

Item 29 is the polynucleotide of any one of the preceding Items, wherein the cytidine deaminase comprises an amino acid sequence with at least 87% identity to SEQ ID NOs: 151.

Item 30 is the polynucleotide of any one of the preceding Items, wherein the cytidine deaminase comprises an amino acid sequence with at least 80% identity to SEQ ID NOs: 152-216.

Item 31 is the polynucleotide of any one of the preceding Items, wherein the cytidine deaminase comprises an amino acid sequence with at least 80% identity to SEQ ID NOs: 14.

Item 32 is the polynucleotide of any one of the preceding Items, the ORF comprises a nucleotide sequence at least 80% identical to SEQ ID NO: 42.

Item 33 is the polynucleotide of any one of the preceding Items, wherein the polynucleotide comprises a 5′ UTR with at least 90% identity to any one of SEQ ID NOs: 391-398.

Item 34 is the polynucleotide of any one of the preceding Items, wherein the polynucleotide comprises a 5′ UTR comprising any one of SEQ ID NOs: 391-398.

Item 35 is the polynucleotide of any one of the preceding Items, wherein the polynucleotide comprises a 3′ UTR with at least 90% identity to any one of SEQ ID NOs: 399-406.

Item 36 is the polynucleotide of any one of the preceding Items, wherein the polynucleotide comprises a 3′ UTR comprising any one of SEQ ID NOs: 399-306.

Item 37 is the polynucleotide of any one of the preceding Items, wherein the polynucleotide comprises a 5′ UTR and a 3′ UTR from the same source.

Item 38 is the polynucleotide of any one of the preceding Items, wherein the polynucleotide comprises a 5′ cap, optionally wherein the 5′ cap is Cap0, Cap1, or Cap2.

Item 39 is the polynucleotide of any one of the preceding Items, wherein at least 75%, 80%, 85%, 90%, 95%, 98%, 99%, or 100% of the codons of the ORF are minimal adenine codons or minimal uridine codons.

Item 40 is the polynucleotide of any one of the preceding Items, wherein the ORF comprises or consists of codons that increase translation of the mRNA in a mammal.

Item 41 is the polynucleotide of any one of the preceding Items, wherein the ORF comprises or consists of codons that increase translation of the mRNA in a human.

Item 42 is the polynucleotide of any one of the preceding Items, wherein the polynucleotide is an mRNA.

Item 43 is the polynucleotide of Item 42, wherein the ORF comprises a sequence having at least 90%, 95%, 98% or 100% identity to any one of SEQ ID NO: 29, 32-41, 224-226, 231-233, 238-240, 245-247, 252-254, 259-261, 266-268, 273-275, 280-282, 287-289, 294-296, 301-303, or 316-321.

Item 44 is the polynucleotide of any one of Items 42-43, wherein at least 10% of the uridine in the mRNA is substituted with a modified uridine.

Item 45 is the polynucleotide of any one of Items 42-43, wherein less than 10% of the uridine in the mRNA is substituted with a modified uridine.

Item 46 is the polynucleotide of Item 45, wherein the modified uridine is one or more of N1-methyl-pseudouridine, pseudouridine, 5-methoxyuridine, or 5-iodouridine.

Item 47 is the polynucleotide of Item 45, wherein the modified uridine is one or both of N1-methyl-pseudouridine or 5-methoxyuridine.

Item 48 is the polynucleotide of any one of Items 45-47, wherein the modified uridine is N1-methyl-pseudouridine.

Item 49 is the polynucleotide of any one of Items 45-47, wherein the modified uridine is 5-methoxyuridine.

Item 50 is the polynucleotide of any one of Items 44, and 36-49, wherein 15% to 45% of the uridine is substituted with the modified uridine.

Item 51 is the polynucleotide of Item 50, wherein at least 20% or at least 30% of the uridine is substituted with the modified uridine.

Item 52 is the polynucleotide of Item 51, wherein at least 80% or at least 90% of the uridine is substituted with the modified uridine.

Item 53 is the polynucleotide of Item 52, wherein 100% of the uridine is substituted with the modified uridine.

Item 54 is the polynucleotide of Item 42, wherein less than 10% of the nucleotides in the mRNA is substituted with a modified nucleotide.

Item 55 is a composition comprising the polynucleotide according to any one of the preceding Items, and at least one guide RNA (gRNA).

Item 56 is a composition comprising a first polynucleotide comprising a first open reading frame (ORF) encoding a polypeptide comprising a cytidine deaminase, optionally an APOBEC3A deaminase, and a NmeCas9 nickase, and a second polynucleotide comprising a second open reading frame encoding a uracil glycosylase inhibitor (UGI), wherein the second polynucleotide is different from the first polynucleotide, and optionally further comprising a guide RNA (gRNA).

Item 57 is the composition of Item 55 or 56, wherein the gRNA is a single guide RNA.

Item 58 is the composition of Item 55 or 56, wherein the gRNA is a dual guide RNA.

Item 59 is a composition comprising the polynucleotide according to any one of Items 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 500; and
   (ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 500; and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 500; and
   (ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

Item 60 is a composition comprising the polynucleotide according to any one of Items 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
   (i) one or more of nucleotides 37-64 is deleted and optionally substituted relative to SEQ ID NO: 500; and
   (ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
   (i) one or more of nucleotides 82-95 is deleted and optionally substituted relative to SEQ ID NO: 500; and
   (ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
   (i) one or more of nucleotides 113-134 is deleted and optionally substituted relative to SEQ ID NO: 500; and
   (ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.

Item 61 is a polypeptide encoded by the polynucleotide of any one of Items 1-60.

Item 62 is a vector comprising the polynucleotide of any one of Items 1-60.

Item 63 is an expression construct comprising a promoter operably linked to a sequence encoding the polynucleotide of any one of Items 1-60.

Item 64 is a plasmid comprising the expression construct of Item 63.

Item 65 is a host cell comprising the vector of Item 62, the expression construct of Item 63, or the plasmid of Item 64.

Item 66 is a pharmaceutical composition comprising the polynucleotide, composition, or polypeptide of any of the preceding Items and a pharmaceutically acceptable carrier.

Item 67 is a kit comprising the polynucleotide, composition, or polypeptide of any of the preceding Items.

Item 68 is use of the polynucleotide, composition, or polypeptide of any one of the preceding Items for modifying a target gene in a cell.

Item 69 is use of the polynucleotide, composition, or polypeptide of any one of the preceding Items for the manufacture of a medicament for modifying a target gene in a cell.

Item 70 is the polynucleotide or composition of any one of the preceding Items, wherein the polynucleotide or composition is formulated as a lipid nucleic acid assembly composition, optionally a lipid nanoparticle.

Item 71 is a method of modifying a target gene comprising delivering to a cell the polynucleotide, polypeptide, or composition of any one of the preceding Items.

Item 72 is a method of modifying a target gene, comprising delivering to the cell one or more lipid nucleic acid assembly compositions, optionally lipid nanoparticles, comprising the polynucleotide according to any one of Items 1-60, and one or more guide RNAs.

Item 73 is the method of any one of Items 71-72, wherein at least one lipid nucleic acid assembly composition comprises lipid nanoparticle (LNPs), optionally wherein all lipid nucleic acid assembly compositions comprise LNPs.

Item 74 is the method of any one of Items 71-72, wherein at least one lipid nucleic acid assembly composition is a lipoplex composition.

Item 75 is the composition or method of any one of Items 72-74, wherein the lipid nucleic acid assembly composition comprises an ionizable lipid.

TABLE 39A
Table of Sequences
The following sequence table provides a listing of sequences disclosed herein. It is understood that if a DNA sequence (comprising Ts)
is referenced with respect to an RNA, then Ts should be replaced with Us (which may be modified or unmodified depending on the context), and
vice versa. * = PS linkage; ′m′ = 2′-O-Me nucleotide. For ORF descriptions, BP = I-pair depleted; GP = E-pair enriched; BS = I-single
depleted; GS = E-single enriched; GCU = subjected to steps of minimizing uridines, minimizing repeats, and maximizing GC content. E-pairs,
I-pairs, E-singles, and I-singles refer, respectively, to the codon pairs or codons of Tables 1-4.

| Description | SEQ ID NO | sequence |
|---|---|---|
| Amino | 1 | MTGAAFKPNPINYILGLDIGIASVGWAMVEIDEEENPIRLIDLGVRVFERAEVPKTGDSLAMARRLARSVRRLTRRRAHRLLR |
| acid | | ARRLLKREGVLQAADFDENGLIKSLPNTPWQLRAAALDRKLTPLEWSAVLLHLIKHRGYLSQRKNEGETADKELGALLKGVAN |
| sequence | | NAHALQTGDFRTPAELALNKFEKESGHIRNQRGDYSHTFSRKDLQAELILLFEKQKEFGNPHVSGGLKEGIETLLMTQRPALS |
| for | | GDAVQKMLGHCTFEPAEPKAAKNTYTAERFIWLTKLNNLRILEQGSERPLTDTERATLMDEPYRKSKLTYAQARKLLGLEDTA |
| Nme2Cas9 | | FFKGLRYGKDNAEASTLMEMKAYHAISRALEKEGLKDKKSPLNLSSELQDEIGTAFSLEKTDEDITGRLKDRVQPEILEALLK |
| encoded | | HISFDKFVQISLKALRRIVPLMEQGKRYDEACAEIYGDHYGKKNTEEKIYLPPIPADEIRNPVVLRALSQARKVINGVVRRYG |
| by mRNA C | | SPARIHIETAREVGKSFKDRKEIEKRQEENRKDREKAAAKFREYFPNFVGEPKSKDILKLRLYEQQHGKCLYSGKEINLVRLN |
| | | EKGYVEIDHALPFSRTWDDSFNNKVLVLGSFNQNKGNQTPYEYENGKDNSREWQEFKARVETSREPRSKKQRILLQKFDEDGE |
| | | KECNLNDTRYVNRFLCQFVADHILLTGKGKRRVFASNGQITNLLRGFWGLRKVRAENDRHHALDAVVVACSTVAMQQKITRFV |
| | | RYKEMNAFDGKTIDKETGKVLHQKTHFPQPWEFFAQEVMIRVFGKPDGKPEFEEADTPEKLRTLLAEKLSSRPEAVHEYVTPL |
| | | FVSRAPNRKMSGAHKDTLRSAKRFVKHNEKISVKRVWLTEIKLADLENMVNYKNGREIELYEALKARLEAYGGNAKQAFDPKD |
| | | NPFYKKGGQLVKAVRVEKTQESGVLLNKKNAYTIADNGDMVRVDVFCKVDKKGKNQYFIVPIYAWQVAENILPDIDCKGYRID |
| | | DSYTFCFSLHKYDLIAFQKDEKSKVEFAYYINCDSSNGRFYLAWHDKGSKEQQFRISTQNLVLIQKYQVNELGKEIRPCRLKK |
| | | RPPVRSGKRTADGSEFESPKKKRKVE |
| Amino | 2 | MEASPASGPRHLMDPHIFTSNENNGIGRHKTYLCYEVERLDNGTSVKMDQHRGFLHNQAKNLLCGFYGRHAELRELDLVPSLQ |
| acid | | LDPAQIYRVTWFISWSPCFSWGCAGEVRAFLQENTHVRLRIFAARIYDYDPLYKEALQMLRDAGAQVSIMTYDEFKHCWDTEV |
| sequence | | DHQGCPFQPWDGLDEHSQALSGRLRAILQNQGNSGSETPGTSESATPESDKKYSIGLAIGTNSVGWAVITDEYKVPSKKFKVL |
| for | | GNTDRHSIKKNLIGALLFDSGETAEATRLKRTARRRYTRRKNRICYLQEIFSNEMAKVDDSFFHRLEESELVEEDKKHERHPI |
| SpyCas9 | | FGNIVDEVAYHEKYPTIYHLRKKLVDSTDKADLRLIYLALAHMIKERGHFLIEGDLNPDNSDVDKLFIQLVQTYNQLFEENPI |
| base | | NASGVDAKAILSARLSKSRRLENLIAQLPGEKKNGLFGNLIALSLGLTPNEKSNEDLAEDAKLQLSKDTYDDDLDNLLAQIGD |
| editor | | QYADLFLAAKNLSDAILLSDILRVNTEITKAPLSASMIKRYDEHHQDLTLLKALVRQQLPEKYKEIFFDQSKNGYAGYIDGGA |
| encoded | | SQEEFYKFIKPILEKMDGTEELLVKLNREDLLRKQRTEDNGSIPHQIHLGELHAILRRQEDFYPFLKDNREKIEKILTFRIPY |
| by mRNA E | | YVGPLARGNSRFAWMTRKSEETITPWNFEEVVDKGASAQSFIERMTNEDKNLPNEKVLPKHSLLYEYFTVYNELTKVKYVTEG |
| | | MRKPAFLSGEQKKAIVDLLFKTNRKVTVKQLKEDYFKKIECFDSVEISGVEDRENASLGTYHDLLKIIKDKDELDNEENEDIL |
| | | EDIVLTLTLFEDREMIEERLKTYAHLFDDKVMKQLKRRRYTGWGRLSRKLINGIRDKQSGKTILDELKSDGFANRNEMQLIHD |
| | | DSLTFKEDIQKAQVSGQGDSLHEHIANLAGSPAIKKGILQTVKVVDELVKVMGRHKPENIVIEMARENQTTQKGQKNSRERMK |
| | | RIEEGIKELGSQILKEHPVENTQLQNEKLYLYYLQNGRDMYVDQELDINRLSDYDVDHIVPQSFLKDDSIDNKVLTRSDKNRG |
| | | KSDNVPSEEVVKKMKNYWRQLLNAKLITQRKEDNLTKAERGGLSELDKAGFIKRQLVETRQITKHVAQILDSRMNTKYDENDK |
| | | LIREVKVITLKSKLVSDERKDFQFYKVREINNYHHAHDAYLNAVVGTALIKKYPKLESEFVYGDYKVYDVRKMIAKSEQEIGK |
| | | ATAKYFFYSNIMNFFKTEITLANGEIRKRPLIETNGETGEIVWDKGRDFATVRKVLSMPQVNIVKKTEVQTGGFSKESILPKR |
| | | NSDKLIARKKDWDPKKYGGFDSPTVAYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYEDTTIDRKRYTSTKEVLDATLIHQSITGLYETRIDLS |
| | | QLGGDGGGSPKKKRKV |
| Amino | 3 | MTNLSDIIEKETGKQLVIQESILMLPEEVEEVIGNKPESDILVHTAYDESTDENVMLLTSDAPEYKPWALVIQDSNGENKIKM |
| acid | | LSGGSKRTADGSEFESPKKKRKVE |
| sequence | | |
| for UGI | | |
| encoded | | |
| by mRNA G | | |
| Amino | 4 | MAAFKPNPINYILGLDIGIASVGWAMVEIDEEENPIRLIDLGVRVFERAEVPKTGDSLAMARRLARSVRRLTRRRAHRLLRAR |
| acid | | RLLKREGVLQAADFDENGLIKSLPNTPWQLRAAALDRKLTPLEWSAVLLHLIKHRGYLSQRKNEGETADKELGALLKGVANNA |
| sequence | | HALQTGDFRTPAELALNKFEKESGHIRNQRGDYSHTFSRKDLQAELILLFEKQKEFGNPHVSGGLKEGIETLLMTQRPALSGD |
| for | | AVQKMLGHCTFEPAEPKAAKNTYTAERFIWLTKLNNLRILEQGSERPLTDTERATLMDEPYRKSKLTYAQARKLLGLEDTAFF |
| Nme2Cas9 | | KGLRYGKDNAEASTLMEMKAYHAISRALEKEGLKDKKSPLNLSSELQDEIGTAFSLEKTDEDITGRLKDRVQPEILEALLKHI |
| encoded | | SFDKFVQISLKALRRIVPLMEQGKRYDEACAEIYGDHYGKKNTEEKIYLPPIPADEIRNPVVLRALSQARKVINGVVRRYGSP |
| by mRNA H | | ARIHIETAREVGKSFKDRKEIEKRQEENRKDREKAAAKFREYFPNFVGEPKSKDILKLRLYEQQHGKCLYSGKEINLVRLNEK |
| | | GYVEIDHALPFSRTWDDSFNNKVLVLGSFNQNKGNQTPYEYENGKDNSREWQEFKARVETSRFPRSKKQRILLQKFDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKSGGGSPKKKRKVSGGSGKNQYFIVPIYAWQVAEN |
| | | ILPDIDCKGYRIDDSYTFCFSLHKYDLIAFQKDEKSKVEFAYYINCDSSNGRFYLAWHDKGSKEQQFRISTQNLVLIQKYQVN |
| | | ELGKEIRPCRLKKRPPVR |
| Amino | 5 | MVPKKKRKVAAFKPNPINYILGLDIGIASVGWAMVEIDEEENPIRLIDLGVRVFERAEVPKTGDSLAMARRLARSVRRLTRRR |
| acid | | AHRLLRARRLLKREGVLQAADFDENGLIKSLPNTPWQLRAAALDRKLTPLEWSAVLLHLIKHRGYLSQRKNEGETADKELGAL |
| sequence | | LKGVANNAHALQTGDFRTPAELALNKFEKESGHIRNQRGDYSHTFSRKDLQAELILLFEKQKEFGNPHVSGGLKEGIETLLMT |
| for | | QRPALSGDAVQKMLGHCTFEPAEPKAAKNTYTAERFIWLTKLNNLRILEQGSERPLTDTERATLMDEPYRKSKLTYAQARKLL |
| Nme2Cas9 | | GLEDTAFFKGLRYGKDNAEASTLMEMKAYHAISRALEKEGLKDKKSPLNLSSELQDEIGTAFSLFKTDEDITGRLKDRVQPEI |
| encoded | | LEALLKHISFDKFVQISLKALRRIVPLMEQGKRYDEACAEIYGDHYGKKNTEEKIYLPPIPADEIRNPVVLRALSQARKVING |
| by mRNA I | | VVRRYGSPARIHIETAREVGKSFKDRKEIEKRQEENRKDREKAAAKFREYFPNFVGEPKSKDILKLRLYEQQHGKCLYSGKEI |
| | | NLVRLNEKGYVEIDHALPESRTWDDSFNNKVLVLGSFNQNKGNQTPYEYENGKDNSREWQEFKARVETSRFPRSKKQRILLQK |
| | | FDEDGEKECNLNDTRYVNRFLCQFVADHILLTGKGKRRVFASNGQITNLLRGFWGLRKVRAENDRHHALDAVVVACSTVAMQQ |
| | | KITRFVRYKEMNAFDGKTIDKETGKVLHQKTHFPQPWEFFAQEVMIRVFGKPDGKPEFEEADTPEKLRTLLAEKLSSRPEAVH |
| | | EYVTPLFVSRAPNRKMSGAHKDTLRSAKRFVKHNEKISVKRVWLTEIKLADLENMVNYKNGREIELYEALKARLEAYGGNAKQ |
| | | AFDPKDNPFYKKGGQLVKAVRVEKTQESGVLLNKKNAYTIADNGDMVRVDVFCKVDKKGKNQYFIVPIYAWQVAENILPDIDC |
| | | KGYRIDDSYTFCFSLHKYDLIAFQKDEKSKVEFAYYINCDSSNGRFYLAWHDKGSKEQQFRISTQNLVLIQKYQVNELGKEIR |
| | | PCRLKKRPPVRYPYDVPDYAAAPAAKKKKLD |
| Amino | 6 | MAAFKPNPINYILGLDIGIASVGWAMVEIDEEENPIRLIDLGVRVFERAEVPKTGDSLAMARRLARSVRRLTRRRAHRLLRAR |
| acid | | RLLKREGVLQAADEDENGLIKSLPNTPWQLRAAALDRKLTPLEWSAVLLHLIKHRGYLSQRKNEGETADKELGALLKGVANNA |
| sequence | | HALQTGDFRTPAELALNKFEKESGHIRNQRGDYSHTFSRKDLQAELILLFEKQKEFGNPHVSGGLKEGIETLLMTQRPALSGD |
| for | | AVQKMLGHCTFEPAEPKAAKNTYTAERFIWLTKLNNLRILEQGSERPLTDTERATLMDEPYRKSKLTYAQARKLLGLEDTAFF |
| Nme2Cas9 | | KGLRYGKDNAEASTLMEMKAYHAISRALEKEGLKDKKSPLNLSSELQDEIGTAFSLEKTDEDITGRLKDRVQPEILEALLKHI |
| encoded | | SFDKFVQISLKALRRIVPLMEQGKRYDEACAEIYGDHYGKKNTEEKIYLPPIPADEIRNPVVLRALSQARKVINGVVRRYGSP |
| by mRNA J | | ARIHIETAREVGKSFKDRKEIEKRQEENRKDREKAAAKFREYFPNFVGEPKSKDILKLRLYEQQHGKCLYSGKEINLVRLNEK |
| | | GYVEIDHALPFSRTWDDSFNNKVLVLGSENQNKGNQTPYEYENGKDNSREWQEFKARVETSRFPRSKKQRILLQKEDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHEPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Amino | 7 | MAAFKPNPINYILGLDIGIASVGWAMVEIDEEENPIRLIDLGVRVFERAEVPKTGDSLAMARRLARSVRRLTRRRAHRLLRAR |
| acid | | RLLKREGVLQAADFDENGLIKSLPNTPWQLRAAALDRKLTPLEWSAVLLHLIKHRGYLSQRKNEGETADKELGALLKGVANNA |
| sequence | | HALQTGDFRTPAELALNKFEKESGHIRNQRGDYSHTFSRKDLQAELILLFEKQKEFGNPHVSGGLKEGIETLLMTQRPALSGD |
| for | | AVQKMLGHCTFEPAEPKAAKNTYTAERFIWLTKLNNLRILEQGSERPLTDTERATLMDEPYRKSKLTYAQARKLLGLEDTAFF |
| Nme2Cas9 | | KGLRYGKDNAEASTLMEMKAYHAISRALEKEGLKDKKSPLNLSSELQDEIGTAFSLEKTDEDITGRLKDRVQPEILEALLKHI |
| encoded | | SFDKFVQISLKALRRIVPLMEQGKRYDEACAEIYGDHYGKKNTEEKIYLPPIPADEIRNPVVLRALSQARKVINGVVRRYGSP |
| by mRNA K | | ARIHIETAREVGKSFKDRKEIEKRQEENRKDREKAAAKFREYFPNFVGEPKSKDILKLRLYEQQHGKCLYSGKEINLVRLNEK |
| | | GYVEIDHALPFSRTWDDSFNNKVLVLGSENQNKGNQTPYEYENGKDNSREWQEFKARVETSRFPRSKKQRILLQKFDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Amino | 8 | MDGSGGGSPKKKRKVGGSGGGAAFKPNPINYILGLDIGIASVGWAMVEIDEEENPIRLIDLGVRVFERAEVPKTGDSLAMARR |
| acid | | LARSVRRLTRRRAHRLLRARRLLKREGVLQAADFDENGLIKSLPNTPWQLRAAALDRKLTPLEWSAVLLHLIKHRGYLSQRKN |
| sequence | | EGETADKELGALLKGVANNAHALQTGDFRTPAELALNKFEKESGHIRNQRGDYSHTFSRKDLQAELILLFEKQKEFGNPHVSG |
| for | | GLKEGIETLLMTQRPALSGDAVQKMLGHCTFEPAEPKAAKNTYTAERFIWLTKLNNLRILEQGSERPLTDTERATLMDEPYRK |
| Nme2Cas9 | | SKLTYAQARKLLGLEDTAFFKGLRYGKDNAEASTLMEMKAYHAISRALEKEGLKDKKSPLNLSSELQDEIGTAFSLEKTDEDI |
| encoded | | TGRLKDRVQPEILEALLKHISFDKFVQISLKALRRIVPLMEQGKRYDEACAEIYGDHYGKKNTEEKIYLPPIPADEIRNPVVL |
| by mRNA L | | RALSQARKVINGVVRRYGSPARIHIETAREVGKSFKDRKEIEKRQEENRKDREKAAAKFREYFPNFVGEPKSKDILKLRLYEQ |
| | | QHGKCLYSGKEINLVRLNEKGYVEIDHALPFSRTWDDSFNNKVLVLGSENQNKGNQTPYEYENGKDNSREWQEFKARVETSRE |
| | | PRSKKQRILLQKFDEDGFKECNLNDTRYVNRFLCQFVADHILLTGKGKRRVFASNGQITNLLRGFWGLRKVRAENDRHHALDA |
| | | VVVACSTVAMQQKITRFVRYKEMNAFDGKTIDKETGKVLHQKTHFPQPWEFFAQEVMIRVEGKPDGKPEFEEADTPEKLRTLL |
| | | AEKLSSRPEAVHEYVTPLFVSRAPNRKMSGAHKDTLRSAKRFVKHNEKISVKRVWLTEIKLADLENMVNYKNGREIELYEALK |
| | | ARLEAYGGNAKQAFDPKDNPFYKKGGQLVKAVRVEKTQESGVLLNKKNAYTIADNGDMVRVDVFCKVDKKGKNQYFIVPIYAW |
| | | QVAENILPDIDCKGYRIDDSYTFCFSLHKYDLIAFQKDEKSKVEFAYYINCDSSNGRFYLAWHDKGSKEQQFRISTQNLVLIQ |
| | | KYQVNELGKEIRPCRLKKRPPVR |
| Amino | 9 | MDGSGGGSPKKKRKVGGSGGGAAFKPNPINYILGLDIGIASVGWAMVEIDEEENPIRLIDLGVRVFERAEVPKTGDSLAMARR |
| acid | | LARSVRRLTRRRAHRLLRARRLLKREGVLQAADEDENGLIKSLPNTPWQLRAAALDRKLTPLEWSAVLLHLIKHRGYLSQRKN |
| sequence | | EGETADKELGALLKGVANNAHALQTGDERTPAELALNKFEKESGHIRNQRGDYSHTFSRKDLQAELILLFEKQKEFGNPHVSG |
| for | | GLKEGIETLLMTQRPALSGDAVQKMLGHCTFEPAEPKAAKNTYTAERFIWLTKLNNLRILEQGSERPLTDTERATLMDEPYRK |
| Nme2Cas9 | | SKLTYAQARKLLGLEDTAFFKGLRYGKDNAEASTLMEMKAYHAISRALEKEGLKDKKSPLNLSSELQDEIGTAFSLEKTDEDI |
| with | | TGRLKDRVQPEILEALLKHISFDKFVQISLKALRRIVPLMEQGKRYDEACAEIYGDHYGKKNTEEKIYLPPIPADEIRNPVVL |
| HiBiT tag | | RALSQARKVINGVVRRYGSPARIHIETAREVGKSFKDRKEIEKRQEENRKDREKAAAKFREYEPNFVGEPKSKDILKLRLYEQ |
| encoded | | QHGKCLYSGKEINLVRLNEKGYVEIDHALPFSRTWDDSFNNKVLVLGSENQNKGNQTPYEYENGKDNSREWQEFKARVETSRF |
| by mRNA M | | PRSKKQRILLQKFDEDGFKECNLNDTRYVNRFLCQFVADHILLTGKGKRRVFASNGQITNLLRGEWGLRKVRAENDRHHALDA |
| | | VVVACSTVAMQQKITRFVRYKEMNAFDGKTIDKETGKVLHQKTHFPQPWEFFAQEVMIRVFGKPDGKPEFEEADTPEKLRTLL |
| | | AEKLSSRPEAVHEYVTPLFVSRAPNRKMSGAHKDTLRSAKRFVKHNEKISVKRVWLTEIKLADLENMVNYKNGREIELYEALK |
| | | ARLEAYGGNAKQAFDPKDNPFYKKGGQLVKAVRVEKTQESGVLLNKKNAYTIADNGDMVRVDVFCKVDKKGKNQYFIVPIYAW |
| | | QVAENILPDIDCKGYRIDDSYTFCFSLHKYDLIAFQKDEKSKVEFAYYINCDSSNGRFYLAWHDKGSKEQQFRISTQNLVLIQ |
| | | KYQVNELGKEIRPCRLKKRPPVRSESATPESVSGWRLEKKIS |
| Amino | 10 | MDGSGGGSPKKKRKVGGSGGGAAFKPNPINYILGLDIGIASVGWAMVEIDEEENPIRLIDLGVRVFERAEVPKTGDSLAMARR |
| acid | | LARSVRRLTRRRAHRLLRARRLLKREGVLQAADFDENGLIKSLPNTPWQLRAAALDRKLTPLEWSAVLLHLIKHRGYLSQRKN |
| sequence | | EGETADKELGALLKGVANNAHALQTGDFRTPAELALNKFEKESGHIRNQRGDYSHTFSRKDLQAELILLFEKQKEFGNPHVSG |
| for | | GLKEGIETLLMTQRPALSGDAVQKMLGHCTFEPAEPKAAKNTYTAERFIWLTKLNNLRILEQGSERPLTDTERATLMDEPYRK |
| Nme2Cas9 | | SKLTYAQARKLLGLEDTAFFKGLRYGKDNAEASTLMEMKAYHAISRALEKEGLKDKKSPLNLSSELQDEIGTAFSLEKTDEDI |
| encoded | | TGRLKDRVQPEILEALLKHISFDKFVQISLKALRRIVPLMEQGKRYDEACAEIYGDHYGKKNTEEKIYLPPIPADEIRNPVVL |
| by mRNA N | | RALSQARKVINGVVRRYGSPARIHIETAREVGKSFKDRKEIEKRQEENRKDREKAAAKFREYFPNFVGEPKSKDILKLRLYEQ |
| | | QHGKCLYSGKEINLVRLNEKGYVEIDHALPFSRTWDDSFNNKVLVLGSENQNKGNQTPYEYENGKDNSREWQEFKARVETSRE |
| | | PRSKKQRILLQKFDEDGEKECNLNDTRYVNRFLCQFVADHILLTGKGKRRVFASNGQITNLLRGFWGLRKVRAENDRHHALDA |
| | | VVVACSTVAMQQKITRFVRYKEMNAFDGKTIDKETGKVLHQKTHFPQPWEFFAQEVMIRVFGKPDGKPEFEEADTPEKLRTLL |
| | | AEKLSSRPEAVHEYVTPLFVSRAPNRKMSGAHKDTLRSAKRFVKHNEKISVKRVWLTEIKLADLENMVNYKNGREIELYEALK |
| | | ARLEAYGGNAKQAFDPKDNPFYKKGGQLVKAVRVEKTQESGVLLNKKNAYTIADNGDMVRVDVFCKVDKKGKNQYFIVPIYAW |
| | | QVAENILPDIDCKGYRIDDSYTFCFSLHKYDLIAFQKDEKSKVEFAYYINCDSSNGRFYLAWHDKGSKEQQFRISTQNLVLIQ |
| | | KYQVNELGKEIRPCRLKKRPPVRSGKRTADGSGGGSPAAKKKKLD |
| Amino | 11 | MDGSGGGSPKKKRKVEDKRPAATKKAGQAKKKKGGSGGGAAFKPNPINYILGLDIGIASVGWAMVEIDEEENPIRLIDLGVRV |
| acid | | FERAEVPKTGDSLAMARRLARSVRRLTRRRAHRLLRARRLLKREGVLQAADEDENGLIKSLPNTPWQLRAAALDRKLTPLEWS |
| sequence | | AVLLHLIKHRGYLSQRKNEGETADKELGALLKGVANNAHALQTGDFRTPAELALNKFEKESGHIRNQRGDYSHTFSRKDLQAE |
| for | | LILLFEKQKEFGNPHVSGGLKEGIETLLMTQRPALSGDAVQKMLGHCTFEPAEPKAAKNTYTAERFIWLTKLNNLRILEQGSE |
| Nme2Cas9 | | RPLTDTERATLMDEPYRKSKLTYAQARKLLGLEDTAFFKGLRYGKDNAEASTLMEMKAYHAISRALEKEGLKDKKSPLNLSSE |
| encoded | | LQDEIGTAFSLEKTDEDITGRLKDRVQPEILEALLKHISFDKFVQISLKALRRIVPLMEQGKRYDEACAEIYGDHYGKKNTEE |
| by mRNA O | | KIYLPPIPADEIRNPVVLRALSQARKVINGVVRRYGSPARIHIETAREVGKSFKDRKEIEKRQEENRKDREKAAAKFREYFPN |
| | | FVGEPKSKDILKLRLYEQQHGKCLYSGKEINLVRLNEKGYVEIDHALPFSRTWDDSFNNKVLVLGSENQNKGNQTPYEYENGK |
| | | DNSREWQEFKARVETSRFPRSKKQRILLQKFDEDGFKECNLNDTRYVNRFLCQFVADHILLTGKGKRRVFASNGQITNLLRGF |
| | | WGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLE |
| | | NMVNYKNGREIELYEALKARLEAYGGNAKQAFDPKDNPFYKKGGQLVKAVRVEKTQESGVLLNKKNAYTIADNGDMVRVDVEC |
| | | KVDKKGKNQYFIVPIYAWQVAENILPDIDCKGYRIDDSYTFCFSLHKYDLIAFQKDEKSKVEFAYYINCDSSNGRFYLAWHDK |
| | | GSKEQQFRISTQNLVLIQKYQVNELGKEIRPCRLKKRPPVR |
| Amino | 12 | MDGSGGGSPKKKRKVEDKRPAATKKAGQAKKKKGGSGGGAAFKPNPINYILGLDIGIASVGWAMVEIDEEENPIRLIDLGVRV |
| acid | | FERAEVPKTGDSLAMARRLARSVRRLTRRRAHRLLRARRLLKREGVLQAADFDENGLIKSLPNTPWQLRAAALDRKLTPLEWS |
| sequence | | AVLLHLIKHRGYLSQRKNEGETADKELGALLKGVANNAHALQTGDFRTPAELALNKFEKESGHIRNQRGDYSHTFSRKDLQAE |
| for | | LILLFEKQKEFGNPHVSGGLKEGIETLLMTQRPALSGDAVQKMLGHCTFEPAEPKAAKNTYTAERFIWLTKLNNLRILEQGSE |
| Nme2Cas9 | | RPLTDTERATLMDEPYRKSKLTYAQARKLLGLEDTAFFKGLRYGKDNAEASTLMEMKAYHAISRALEKEGLKDKKSPLNLSSE |
| with | | LQDEIGTAFSLEKTDEDITGRLKDRVQPEILEALLKHISFDKFVQISLKALRRIVPLMEQGKRYDEACAEIYGDHYGKKNTEE |
| HiBiT tag | | KIYLPPIPADEIRNPVVLRALSQARKVINGVVRRYGSPARIHIETAREVGKSFKDRKEIEKRQEENRKDREKAAAKFREYFPN |
| encoded | | FVGEPKSKDILKLRLYEQQHGKCLYSGKEINLVRLNEKGYVEIDHALPFSRTWDDSFNNKVLVLGSENQNKGNQTPYEYENGK |
| by mRNA P | | DNSREWQEFKARVETSRFPRSKKQRILLQKFDEDGFKECNLNDTRYVNRFLCQFVADHILLTGKGKRRVFASNGQITNLLRGF |
| | | WGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLE |
| | | NMVNYKNGREIELYEALKARLEAYGGNAKQAFDPKDNPFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKYDLIAFQKDEKSKVEFAYYINCDSSNGRFYLAWHDK |
| | | GSKEQQFRISTQNLVLIQKYQVNELGKEIRPCRLKKRPPVRSESATPESVSGWRLFKKIS |
| Amino | 13 | MDGSGGGSEDKRPAATKKAGQAKKKKGGSGGGAAFKPNPINYILGLDIGIASVGWAMVEIDEEENPIRLIDLGVRVFERAEVP |
| acid | | KTGDSLAMARRLARSVRRLTRRRAHRLLRARRLLKREGVLQAADFDENGLIKSLPNTPWQLRAAALDRKLTPLEWSAVLLHLI |
| sequence | | KHRGYLSQRKNEGETADKELGALLKGVANNAHALQTGDERTPAELALNKFEKESGHIRNQRGDYSHTFSRKDLQAELILLFEK |
| for | | QKEFGNPHVSGGLKEGIETLLMTQRPALSGDAVQKMLGHCTFEPAEPKAAKNTYTAERFIWLTKLNNLRILEQGSERPLTDTE |
| Nme2Cas9 | | RATLMDEPYRKSKLTYAQARKLLGLEDTAFFKGLRYGKDNAEASTLMEMKAYHAISRALEKEGLKDKKSPLNLSSELQDEIGT |
| encoded | | AFSLFKTDEDITGRLKDRVQPEILEALLKHISFDKFVQISLKALRRIVPLMEQGKRYDEACAEIYGDHYGKKNTEEKIYLPPI |
| by mRNA Q | | PADEIRNPVVLRALSQARKVINGVVRRYGSPARIHIETAREVGKSFKDRKEIEKRQEENRKDREKAAAKFREYFPNFVGEPKS |
| | | KDILKLRLYEQQHGKCLYSGKEINLVRLNEKGYVEIDHALPFSRTWDDSFNNKVLVLGSENQNKGNQTPYEYENGKDNSREWQ |
| | | EFKARVETSRFPRSKKQRILLQKFDEDGEKECNLNDTRYVNRFLCQFVADHILLTGKGKRRVFASNGQITNLLRGFWGLRKVR |
| | | AENDRHHALDAVVVACSTVAMQQKITRFVRYKEMNAFDGKTIDKETGKVLHQKTHFPQPWEFFAQEVMIRVFGKPDGKPEFEE |
| | | ADTPEKLRTLLAEKLSSRPEAVHEYVTPLFVSRAPNRKMSGAHKDTLRSAKRFVKHNEKISVKRVWLTEIKLADLENMVNYKN |
| | | GREIELYEALKARLEAYGGNAKQAFDPKDNPFYKKGGQLVKAVRVEKTQESGVLLNKKNAYTIADNGDMVRVDVFCKVDKKGK |
| | | NQYFIVPIYAWQVAENILPDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVR |
| Amino | 14 | MDGSGGGSPKKKRKVEDKRPAATKKAGQAKKKKGGSGGGEASPASGPRHLMDPHIFTSNENNGIGRHKTYLCYEVERLDNGTS |
| acid | | VKMDQHRGFLHNQAKNLLCGFYGRHAELRFLDLVPSLQLDPAQIYRVTWFISWSPCFSWGCAGEVRAFLQENTHVRLRIFAAR |
| sequence | | IYDYDPLYKEALQMLRDAGAQVSIMTYDEFKHCWDTFVDHQGCPFQPWDGLDEHSQALSGRLRAILQNQGNSGSETPGTSESA |
| for | | TPESAAFKPNPINYILGLAIGIASVGWAMVEIDEEENPIRLIDLGVRVFERAEVPKTGDSLAMARRLARSVRRLTRRRAHRLL |
| Nme2Cas9 | | RARRLLKREGVLQAADFDENGLIKSLPNTPWQLRAAALDRKLTPLEWSAVLLHLIKHRGYLSQRKNEGETADKELGALLKGVA |
| base | | NNAHALQTGDFRTPAELALNKFEKESGHIRNQRGDYSHTFSRKDLQAELILLFEKQKEFGNPHVSGGLKEGIETLLMTQRPAL |
| editor | | SGDAVQKMLGHCTFEPAEPKAAKNTYTAERFIWLTKLNNLRILEQGSERPLTDTERATLMDEPYRKSKLTYAQARKLLGLEDT |
| encoded | | AFFKGLRYGKDNAEASTLMEMKAYHAISRALEKEGLKDKKSPLNLSSELQDEIGTAFSLEKTDEDITGRLKDRVQPEILEALL |
| by mRNA R | | KHISFDKFVQISLKALRRIVPLMEQGKRYDEACAEIYGDHYGKKNTEEKIYLPPIPADEIRNPVVLRALSQARKVINGVVRRY |
| | | GSPARIHIETAREVGKSFKDRKEIEKRQEENRKDREKAAAKFREYFPNFVGEPKSKDILKLRLYEQQHGKCLYSGKEINLVRL |
| | | NEKGYVEIDHALPFSRTWDDSFNNKVLVLGSENQNKGNQTPYEYENGKDNSREWQEFKARVETSRFPRSKKQRILLQKFDEDG |
| | | FKECNLNDTRYVNRFLCQFVADHILLTGKGKRRVFASNGQITNLLRGFWGLRKVRAENDRHHALDAVVVACSTVAMQQKITRF |
| | | VRYKEMNAFDGKTIDKETGKVLHQKTHFPQPWEFFAQEVMIRVFGKPDGKPEFEEADTPEKLRTLLAEKLSSRPEAVHEYVTP |
| | | LFVSRAPNRKMSGAHKDTLRSAKRFVKHNEKISVKRVWLTEIKLADLENMVNYKNGREIELYEALKARLEAYGGNAKQAFDPK |
| | | DNPFYKKGGQLVKAVRVEKTQESGVLLNKKNAYTIADNGDMVRVDVFCKVDKKGKNQYFIVPIYAWQVAENILPDIDCKGYRI |
| | | DDSYTFCFSLHKYDLIAFQKDEKSKVEFAYYINCDSSNGRFYLAWHDKGSKEQQFRISTQNLVLIQKYQVNELGKEIRPCRLK |
| | | KRPPVR |
| mRNA C | 15 | GGGAAGCUCAGAAUAAACGCUCAACUUUGGCCGGAUCUGCCACCAUGACCGGUGCCGCCUUCAAGCCCAACCCCAUCAACUAC |
| encoding | | AUCCUGGGCCUGGACAUCGGCAUCGCCUCCGUGGGCUGGGCCAUGGUGGAGAUCGACGAGGAGGAGAACCCCAUCCGGCUGAU |
| Nme2Cas9 | | CGACCUGGGCGUGCGGGUGUUCGAGCGGGCCGAGGUGCCCAAGACCGGCGACUCCCUGGCCAUGGCCCGGCGGCUGGCCCGGU |
| | | CCGUGCGGCGGCUGACCCGGCGGCGGGCCCACCGGCUGCUGCGGGCCCGGCGGCUGCUGAAGCGGGAGGGCGUGCUGCAGGCC |
| | | GCCGACUUCGACGAGAACGGCCUGAUCAAGUCCCUGCCCAACACCCCCUGGCAGCUGCGGGCCGCCGCCCUGGACCGGAAGCU |
| | | GACCCCCCUGGAGUGGUCCGCCGUGCUGCUGCACCUGAUCAAGCACCGGGGCUACCUGUCCCAGCGGAAGAACGAGGGCGAGA |
| | | CCGCCGACAAGGAGCUGGGCGCCCUGCUGAAGGGCGUGGCCAACAACGCCCACGCCCUGCAGACCGGCGACUUCCGGACCCCC |
| | | GCCGAGCUGGCCCUGAACAAGUUCGAGAAGGAGUCCGGCCACAUCCGGAACCAGCGGGGCGACUACUCCCACACCUUCUCCCG |
| | | GAAGGACCUGCAGGCCGAGCUGAUCCUGCUGUUCGAGAAGCAGAAGGAGUUCGGCAACCCCCACGUGUCCGGCGGCCUGAAGG |
| | | AGGGCAUCGAGACCCUGCUGAUGACCCAGCGGCCCGCCCUGUCCGGCGACGCCGUGCAGAAGAUGCUGGGCCACUGCACCUUC |
| | | GAGCCCGCCGAGCCCAAGGCCGCCAAGAACACCUACACCGCCGAGCGGUUCAUCUGGCUGACCAAGCUGAACAACCUGCGGAU |
| | | CCUGGAGCAGGGCUCCGAGCGGCCCCUGACCGACACCGAGCGGGCCACCCUGAUGGACGAGCCCUACCGGAAGUCCAAGCUGA |
| | | CCUACGCCCAGGCCCGGAAGCUGCUGGGCCUGGAGGACACCGCCUUCUUCAAGGGCCUGCGGUACGGCAAGGACAACGCCGAG |
| | | GCCUCCACCCUGAUGGAGAUGAAGGCCUACCACGCCAUCUCCCGGGCCCUGGAGAAGGAGGGCCUGAAGGACAAGAAGUCCCC |
| | | CCUGAACCUGUCCUCCGAGCUGCAGGACGAGAUCGGCACCGCCUUCUCCCUGUUCAAGACCGACGAGGACAUCACCGGCCGGC |
| | | UGAAGGACCGGGUGCAGCCCGAGAUCCUGGAGGCCCUGCUGAAGCACAUCUCCUUCGACAAGUUCGUGCAGAUCUCCCUGAAG |
| | | GCCCUGCGGCGGAUCGUGCCCCUGAUGGAGCAGGGCAAGCGGUACGACGAGGCCUGCGCCGAGAUCUACGGCGACCACUACGG |
| | | CAAGAAGAACACCGAGGAGAAGAUCUACCUGCCCCCCAUCCCCGCCGACGAGAUCCGGAACCCCGUGGUGCUGCGGGCCCUGU |
| | | CCCAGGCCCGGAAGGUGAUCAACGGCGUGGUGCGGCGGUACGGCUCCCCCGCCCGGAUCCACAUCGAGACCGCCCGGGAGGUG |
| | | GGCAAGUCCUUCAAGGACCGGAAGGAGAUCGAGAAGCGGCAGGAGGAGAACCGGAAGGACCGGGAGAAGGCCGCCGCCAAGUU |
| | | CCGGGAGUACUUCCCCAACUUCGUGGGCGAGCCCAAGUCCAAGGACAUCCUGAAGCUGCGGCUGUACGAGCAGCAGCACGGCA |
| | | AGUGCCUGUACUCCGGCAAGGAGAUCAACCUGGUGCGGCUGAACGAGAAGGGCUACGUGGAGAUCGACCACGCCCUGCCCUUC |
| | | UCCCGGACCUGGGACGACUCCUUCAACAACAAGGUGCUGGUGCUGGGCUCCGAGAACCAGAACAAGGGCAACCAGACCCCCUA |
| | | CGAGUACUUCAACGGCAAGGACAACUCCCGGGAGUGGCAGGAGUUCAAGGCCCGGGUGGAGACCUCCCGGUUCCCCCGGUCCA |
| | | AGAAGCAGCGGAUCCUGCUGCAGAAGUUCGACGAGGACGGCUUCAAGGAGUGCAACCUGAACGACACCCGGUACGUGAACCGC |
| | | UUCCUGUGCCAGUUCGUGGCCGACCACAUCCUGCUGACCGGCAAGGGCAAGCGGCGGGUGUUCGCCUCCAACGGCCAGAUCAC |
| | | CAACCUGCUGCGGGGCUUCUGGGGCCUGCGGAAGGUGCGGGCCGAGAACGACCGGCACCACGCCCUGGACGCCGUGGUGGUGG |
| | | CCUGCUCCACCGUGGCCAUGCAGCAGAAGAUCACCCGGUUCGUGCGGUACAAGGAGAUGAACGCCUUCGACGGCAAGACCAUC |
| | | GACAAGGAGACCGGCAAGGUGCUGCACCAGAAGACCCACUUCCCCCAGCCCUGGGAGUUCUUCGCCCAGGAGGUGAUGAUCCG |
| | | GGUGUUCGGCAAGCCCGACGGCAAGCCCGAGUUCGAGGAGGCCGACACCCCCGAGAAGCUGCGGACCCUGCUGGCCGAGAAGC |
| | | UGUCCUCCCGGCCCGAGGCCGUGCACGAGUACGUGACCCCCCUGUUCGUGUCCCGGGCCCCCAACCGGAAGAUGUCCGGCGCC |
| | | CACAAGGACACCCUGCGGUCCGCCAAGCGGUUCGUGAAGCACAACGAGAAGAUCUCCGUGAAGCGGGUGUGGCUGACCGAGAU |
| | | CAAGCUGGCCGACCUGGAGAACAUGGUGAACUACAAGAACGGCCGGGAGAUCGAGCUGUACGAGGCCCUGAAGGCCCGGCUGG |
| | | AGGCCUACGGCGGCAACGCCAAGCAGGCCUUCGACCCCAAGGACAACCCCUUCUACAAGAAGGGCGGCCAGCUGGUGAAGGCC |
| | | GUGCGGGUGGAGAAGACCCAGGAGUCCGGCGUGCUGCUGAACAAGAAGAACGCCUACACCAUCGCCGACAACGGCGACAUGGU |
| | | GCGGGUGGACGUGUUCUGCAAGGUGGACAAGAAGGGCAAGAACCAGUACUUCAUCGUGCCCAUCUACGCCUGGCAGGUGGCCG |
| | | AGAACAUCCUGCCCGACAUCGACUGCAAGGGCUACCGGAUCGACGACUCCUACACCUUCUGCUUCUCCCUGCACAAGUACGAC |
| | | CUGAUCGCCUUCCAGAAGGACGAGAAGUCCAAGGUGGAGUUCGCCUACUACAUCAACUGCGACUCCUCCAACGGCCGGUUCUA |
| | | CCUGGCCUGGCACGACAAGGGCUCCAAGGAGCAGCAGUUCCGGAUCUCCACCCAGAACCUGGUGCUGAUCCAGAAGUACCAGG |
| | | UGAACGAGCUGGGCAAGGAGAUCCGGCCCUGCCGGCUGAAGAAGCGGCCCCCCGUGCGGUCCGGAAAGCGGACCGCCGACGGC |
| | | UCCGAGUUCGAGUCCCCCAAGAAGAAGCGGAAGGUGGAGUAGCUAGCACCAGCCUCAAGAACACCCGAAUGGAGUCUCUAAGC |
| | | UACAUAAUACCAACUUACACUUUACAAAAUGUUGUCCCCCAAAAUGUAGCCAUUCGUAUCUGCUCCUAAUAAAAAGAAAGUUU |
| | | CUUCACAUUCUCUCGAGAAAAAAAAAAAAUGGAAAAAAAAAAAACGGAAAAAAAAAAAAGGUAAAAAAAAAAAAUAUAAAAAA |
| | | AAAAAACAUAAAAAAAAAAAACGAAAAAAAAAAAACGUAAAAAAAAAAAACUCAAAAAAAAAAAAGAUAAAAAAAAAAAACCU |
| | | AAAAAAAAAAAAUGUAAAAAAAAAAAAGGGAAAAAAAAAAAACGCAAAAAAAAAAAACACAAAAAAAAAAAAUGCAAAAAAAA |
| | | AAAAUCGAAAAAAAAAAAAUCUAAAAAAAAAAAACGAAAAAAAAAAAACCCAAAAAAAAAAAAGACAAAAAAAAAAAAUAGAA |
| | | AAAAAAAAAAGUUAAAAAA |
| mRNA E | 16 | GGGAAGCUCAGAAUAAACGCUCAACUUUGGCCGGAUCUGCCACCAUGGAGGCCUCCCCCGCCUCCGGCCCCCGGCACCUGAUG |
| encoding | | GACCCCCACAUCUUCACCUCCAACUUCAACAACGGCAUCGGCCGGCACAAGACCUACCUGUGCUACGAGGUGGAGCGGCUGGA |
| SpyCas9 | | CAACGGCACCUCCGUGAAGAUGGACCAGCACCGGGGCUUCCUGCACAACCAGGCCAAGAACCUGCUGUGCGGCUUCUACGGCC |
| base | | GGCACGCCGAGCUGCGGUUCCUGGACCUGGUGCCCUCCCUGCAGCUGGACCCCGCCCAGAUCUACCGGGUGACCUGGUUCAUC |
| editor | | UCCUGGUCCCCCUGCUUCUCCUGGGGCUGCGCCGGCGAGGUGCGGGCCUUCCUGCAGGAGAACACCCACGUGCGGCUGCGGAU |
| | | CUUCGCCGCCCGGAUCUACGACUACGACCCCCUGUACAAGGAGGCCCUGCAGAUGCUGCGGGACGCCGGCGCCCAGGUGUCCA |
| | | UCAUGACCUACGACGAGUUCAAGCACUGCUGGGACACCUUCGUGGACCACCAGGGCUGCCCCUUCCAGCCCUGGGACGGCCUG |
| | | GACGAGCACUCCCAGGCCCUGUCCGGCCGGCUGCGGGCCAUCCUGCAGAACCAGGGCAACUCCGGCUCCGAGACCCCCGGCAC |
| | | CUCCGAGUCCGCCACCCCCGAGUCCGACAAGAAGUACUCCAUCGGCCUGGCCAUCGGCACCAACUCCGUGGGCUGGGCCGUGA |
| | | UCACCGACGAGUACAAGGUGCCCUCCAAGAAGUUCAAGGUGCUGGGCAACACCGACCGGCACUCCAUCAAGAAGAACCUGAUC |
| | | GGCGCCCUGCUGUUCGACUCCGGCGAGACCGCCGAGGCCACCCGGCUGAAGCGGACCGCCCGGCGGCGGUACACCCGGCGGAA |
| | | GAACCGGAUCUGCUACCUGCAGGAGAUCUUCUCCAACGAGAUGGCCAAGGUGGACGACUCCUUCUUCCACCGGCUGGAGGAGU |
| | | CCUUCCUGGUGGAGGAGGACAAGAAGCACGAGCGGCACCCCAUCUUCGGCAACAUCGUGGACGAGGUGGCCUACCACGAGAAG |
| | | UACCCCACCAUCUACCACCUGCGGAAGAAGCUGGUGGACUCCACCGACAAGGCCGACCUGCGGCUGAUCUACCUGGCCCUGGC |
| | | CCACAUGAUCAAGUUCCGGGGCCACUUCCUGAUCGAGGGCGACCUGAACCCCGACAACUCCGACGUGGACAAGCUGUUCAUCC |
| | | AGCUGGUGCAGACCUACAACCAGCUGUUCGAGGAGAACCCCAUCAACGCCUCCGGCGUGGACGCCAAGGCCAUCCUGUCCGCC |
| | | CGGCUGUCCAAGUCCCGGCGGCUGGAGAACCUGAUCGCCCAGCUGCCCGGCGAGAAGAAGAACGGCCUGUUCGGCAACCUGAU |
| | | CGCCCUGUCCCUGGGCCUGACCCCCAACUUCAAGUCCAACUUCGACCUGGCCGAGGACGCCAAGCUGCAGCUGUCCAAGGACA |
| | | CCUACGACGACGACCUGGACAACCUGCUGGCCCAGAUCGGCGACCAGUACGCCGACCUGUUCCUGGCCGCCAAGAACCUGUCC |
| | | GACGCCAUCCUGCUGUCCGACAUCCUGCGGGUGAACACCGAGAUCACCAAGGCCCCCCUGUCCGCCUCCAUGAUCAAGCGGUA |
| | | CGACGAGCACCACCAGGACCUGACCCUGCUGAAGGCCCUGGUGCGGCAGCAGCUGCCCGAGAAGUACAAGGAGAUCUUCUUCG |
| | | ACCAGUCCAAGAACGGCUACGCCGGCUACAUCGACGGCGGCGCCUCCCAGGAGGAGUUCUACAAGUUCAUCAAGCCCAUCCUG |
| | | GAGAAGAUGGACGGCACCGAGGAGCUGCUGGUGAAGCUGAACCGGGAGGACCUGCUGCGGAAGCAGCGGACCUUCGACAACGG |
| | | CUCCAUCCCCCACCAGAUCCACCUGGGCGAGCUGCACGCCAUCCUGCGGCGGCAGGAGGACUUCUACCCCUUCCUGAAGGACA |
| | | ACCGGGAGAAGAUCGAGAAGAUCCUGACCUUCCGGAUCCCCUACUACGUGGGCCCCCUGGCCCGGGGCAACUCCCGGUUCGCC |
| | | UGGAUGACCCGGAAGUCCGAGGAGACCAUCACCCCCUGGAACUUCGAGGAGGUGGUGGACAAGGGCGCCUCCGCCCAGUCCUU |
| | | CAUCGAGCGGAUGACCAACUUCGACAAGAACCUGCCCAACGAGAAGGUGCUGCCCAAGCACUCCCUGCUGUACGAGUACUUCA |
| | | CCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUG |
| | | CUUCGACUCCGUGGAGAUCUCCGGCGUGGAGGACCGGUUCAACGCCUCCCUGGGCACCUACCACGACCUGCUGAAGAUCAUCA |
| | | AGGACAAGGACUUCCUGGACAACGAGGAGAACGAGGACAUCCUGGAGGACAUCGUGCUGACCCUGACCCUGUUCGAGGACCGG |
| | | GAGAUGAUCGAGGAGCGGCUGAAGACCUACGCCCACCUGUUCGACGACAAGGUGAUGAAGCAGCUGAAGCGGCGGCGGUACAC |
| | | CGGCUGGGGCCGGCUGUCCCGGAAGCUGAUCAACGGCAUCCGGGACAAGCAGUCCGGCAAGACCAUCCUGGACUUCCUGAAGU |
| | | CCGACGGCUUCGCCAACCGGAACUUCAUGCAGCUGAUCCACGACGACUCCCUGACCUUCAAGGAGGACAUCCAGAAGGCCCAG |
| | | GUGUCCGGCCAGGGCGACUCCCUGCACGAGCACAUCGCCAACCUGGCCGGCUCCCCCGCCAUCAAGAAGGGCAUCCUGCAGAC |
| | | CGUGAAGGUGGUGGACGAGCUGGUGAAGGUGAUGGGCCGGCACAAGCCCGAGAACAUCGUGAUCGAGAUGGCCCGGGAGAACC |
| | | AGACCACCCAGAAGGGCCAGAAGAACUCCCGGGAGCGGAUGAAGCGGAUCGAGGAGGGCAUCAAGGAGCUGGGCUCCCAGAUC |
| | | CUGAAGGAGCACCCCGUGGAGAACACCCAGCUGCAGAACGAGAAGCUGUACCUGUACUACCUGCAGAACGGCCGGGACAUGUA |
| | | CGUGGACCAGGAGCUGGACAUCAACCGGCUGUCCGACUACGACGUGGACCACAUCGUGCCCCAGUCCUUCCUGAAGGACGACU |
| | | CCAUCGACAACAAGGUGCUGACCCGGUCCGACAAGAACCGGGGCAAGUCCGACAACGUGCCCUCCGAGGAGGUGGUGAAGAAG |
| | | AUGAAGAACUACUGGCGGCAGCUGCUGAACGCCAAGCUGAUCACCCAGCGGAAGUUCGACAACCUGACCAAGGCCGAGCGGGG |
| | | CGGCCUGUCCGAGCUGGACAAGGCCGGCUUCAUCAAGCGGCAGCUGGUGGAGACCCGGCAGAUCACCAAGCACGUGGCCCAGA |
| | | UCCUGGACUCCCGGAUGAACACCAAGUACGACGAGAACGACAAGCUGAUCCGGGAGGUGAAGGUGAUCACCCUGAAGUCCAAG |
| | | CUGGUGUCCGACUUCCGGAAGGACUUCCAGUUCUACAAGGUGCGGGAGAUCAACAACUACCACCACGCCCACGACGCCUACCU |
| | | GAACGCCGUGGUGGGCACCGCCCUGAUCAAGAAGUACCCCAAGCUGGAGUCCGAGUUCGUGUACGGCGACUACAAGGUGUACG |
| | | ACGUGCGGAAGAUGAUCGCCAAGUCCGAGCAGGAGAUCGGCAAGGCCACCGCCAAGUACUUCUUCUACUCCAACAUCAUGAAC |
| | | UUCUUCAAGACCGAGAUCACCCUGGCCAACGGCGAGAUCCGGAAGCGGCCCCUGAUCGAGACCAACGGCGAGACCGGCGAGAU |
| | | CGUGUGGGACAAGGGCCGGGACUUCGCCACCGUGCGGAAGGUGCUGUCCAUGCCCCAGGUGAACAUCGUGAAGAAGACCGAGG |
| | | UGCAGACCGGCGGCUUCUCCAAGGAGUCCAUCCUGCCCAAGCGGAACUCCGACAAGCUGAUCGCCCGGAAGAAGGACUGGGAC |
| | | CCCAAGAAGUACGGCGGCUUCGACUCCCCCACCGUGGCCUACUCCGUGCUGGUGGUGGCCAAGGUGGAGAAGGGCAAGUCCAA |
| | | GAAGCUGAAGUCCGUGAAGGAGCUGCUGGGCAUCACCAUCAUGGAGCGGUCCUCCUUCGAGAAGAACCCCAUCGACUUCCUGG |
| | | AGGCCAAGGGCUACAAGGAGGUGAAGAAGGACCUGAUCAUCAAGCUGCCCAAGUACUCCCUGUUCGAGCUGGAGAACGGCCGG |
| | | AAGCGGAUGCUGGCCUCCGCCGGCGAGCUGCAGAAGGGCAACGAGCUGGCCCUGCCCUCCAAGUACGUGAACUUCCUGUACCU |
| | | GGCCUCCCACUACGAGAAGCUGAAGGGCUCCCCCGAGGACAACGAGCAGAAGCAGCUGUUCGUGGAGCAGCACAAGCACUACC |
| | | UGGACGAGAUCAUCGAGCAGAUCUCCGAGUUCUCCAAGCGGGUGAUCCUGGCCGACGCCAACCUGGACAAGGUGCUGUCCGCC |
| | | UACAACAAGCACCGGGACAAGCCCAUCCGGGAGCAGGCCGAGAACAUCAUCCACCUGUUCACCCUGACCAACCUGGGCGCCCC |
| | | CGCCGCCUUCAAGUACUUCGACACCACCAUCGACCGGAAGCGGUACACCUCCACCAAGGAGGUGCUGGACGCCACCCUGAUCC |
| | | ACCAGUCCAUCACCGGCCUGUACGAGACCCGGAUCGACCUGUCCCAGCUGGGCGGCGACGGCGGCGGCUCCCCCAAGAAGAAG |
| | | CGGAAGGUGUGACUAGCACCAGCCUCAAGAACACCCGAAUGGAGUCUCUAAGCUACAUAAUACCAACUUACACUUUACAAAAU |
| | | GUUGUCCCCCAAAAUGUAGCCAUUCGUAUCUGCUCCUAAUAAAAAGAAAGUUUCUUCACAUUCUCUCGAGAAAAAAAAAAAAU |
| | | GGAAAAAAAAAAAACGGAAAAAAAAAAAAGGUAAAAAAAAAAAAUAUAAAAAAAAAAAACAUAAAAAAAAAAAACGAAAAAAA |
| | | AAAAACGUAAAAAAAAAAAACUCAAAAAAAAAAAAGAUAAAAAAAAAAAACCUAAAAAAAAAAAAUGUAAAAAAAAAAAAGGG |
| | | AAAAAAAAAAAACGCAAAAAAAAAAAACACAAAAAAAAAAAAUGCAAAAAAAAAAAAUCGAAAAAAAAAAAAUCUAAAAAAAA |
| | | AAAACGAAAAAAAAAAAACCCAAAAAAAAAAAAGACAAAAAAAAAAAAUAGAAAAAAAAAAAAGUUAAAAAAAAAAAACUGAA |
| | | AAAAAAAAAAUUUAAAAAAAAAAAAUCUAG |
| mRNA G | 17 | GGGAAGCUCAGAAUAAACGCUCAACUUUGGCCGGAUCUGCCACCAUGACCAACCUGUCCGACAUCAUCGAGAAGGAGACCGGC |
| encoding | | AAGCAGCUGGUGAUCCAGGAGUCCAUCCUGAUGCUGCCCGAGGAGGUGGAGGAGGUGAUCGGCAACAAGCCCGAGUCCGACAU |
| UGI | | CCUGGUGCACACCGCCUACGACGAGUCCACCGACGAGAACGUGAUGCUGCUGACCUCCGACGCCCCCGAGUACAAGCCCUGGG |
| | | CCCUGGUGAUCCAGGACUCCAACGGCGAGAACAAGAUCAAGAUGCUGUCCGGCGGCUCCAAGCGGACCGCCGACGGCUCCGAG |
| | | UUCGAGUCCCCCAAGAAGAAGCGGAAGGUGGAGUGAUAGCUAGCACCAGCCUCAAGAACACCCGAAUGGAGUCUCUAAGCUAC |
| | | AUAAUACCAACUUACACUUUACAAAAUGUUGUCCCCCAAAAUGUAGCCAUUCGUAUCUGCUCCUAAUAAAAAGAAAGUUUCUU |
| | | CACAUUCUCUCGAGAAAAAAAAAAAAUGGAAAAAAAAAAAACGGAAAAAAAAAAAAGGUAAAAAAAAAAAAUAUAAAAAAAAA |
| | | AAACAUAAAAAAAAAAAACGAAAAAAAAAAAACGUAAAAAAAAAAAACUCAAAAAAAAAAAAGAUAAAAAAAAAAAACCUAAA |
| | | AAAAAAAAAUGUAAAAAAAAAAAAGGGAAAAAAAAAAAACGCAAAAAAAAAAAACACAAAAAAAAAAAAUGCAAAAAAAAAAA |
| | | AUCGAAAAAAAAAAAAUCUAAAAAAAAAAAACGAAAAAAAAAAAACCCAAAAAAAAAAAAGACAAAAAAAAAAAAUAGAAAAA |
| | | AAAAAAAGUUAAAAAAAAAAAACUGAAAAAAAAAAAAUUUAAAAAAAAAAAAUCUAG |
| mRNA H | 18 | GGGaagctcagaataaacgctcaactttggccggatctgccacCATGGCCGCCTTCAAGCCCAACCCCATCAACTACATCCTG |
| encoding | | GGCCTGGACATCGGCATCGCCTCCGTGGGCTGGGCCATGGTGGAGATCGACGAGGAGGAGAACCCCATCCGGCTGATCGACCT |
| Nme2Cas9 | | GGGCGTGCGGGTGTTCGAGCGGGCCGAGGTGCCCAAGACCGGCGACTCCCTGGCCATGGCCCGGCGGCTGGCCCGGTCCGTGC |
| | | GGCGGCTGACCCGGCGGCGGGCCCACCGGCTGCTGCGGGCCCGGCGGCTGCTGAAGCGGGAGGGCGTGCTGCAGGCCGCCGAC |
| | | TTCGACGAGAACGGCCTGATCAAGTCCCTGCCCAACACCCCCTGGCAGCTGCGGGCCGCCGCCCTGGACCGGAAGCTGACCCC |
| | | CCTGGAGTGGTCCGCCGTGCTGCTGCACCTGATCAAGCACCGGGGCTACCTGTCCCAGCGGAAGAACGAGGGCGAGACCGCCG |
| | | ACAAGGAGCTGGGCGCCCTGCTGAAGGGCGTGGCCAACAACGCCCACGCCCTGCAGACCGGCGACTTCCGGACCCCCGCCGAG |
| | | CTGGCCCTGAACAAGTTCGAGAAGGAGTCCGGCCACATCCGGAACCAGCGGGGCGACTACTCCCACACCTTCTCCCGGAAGGA |
| | | CCTGCAGGCCGAGCTGATCCTGCTGTTCGAGAAGCAGAAGGAGTTCGGCAACCCCCACGTGTCCGGCGGCCTGAAGGAGGGCA |
| | | TCGAGACCCTGCTGATGACCCAGCGGCCCGCCCTGTCCGGCGACGCCGTGCAGAAGATGCTGGGCCACTGCACCTTCGAGCCC |
| | | GCCGAGCCCAAGGCCGCCAAGAACACCTACACCGCCGAGCGGTTCATCTGGCTGACCAAGCTGAACAACCTGCGGATCCTGGA |
| | | GCAGGGCTCCGAGCGGCCCCTGACCGACACCGAGCGGGCCACCCTGATGGACGAGCCCTACCGGAAGTCCAAGCTGACCTACG |
| | | CCCAGGCCCGGAAGCTGCTGGGCCTGGAGGACACCGCCTTCTTCAAGGGCCTGCGGTACGGCAAGGACAACGCCGAGGCCTCC |
| | | ACCCTGATGGAGATGAAGGCCTACCACGCCATCTCCCGGGCCCTGGAGAAGGAGGGCCTGAAGGACAAGAAGTCCCCCCTGAA |
| | | CCTGTCCTCCGAGCTGCAGGACGAGATCGGCACCGCCTTCTCCCTGTTCAAGACCGACGAGGACATCACCGGCCGGCTGAAGG |
| | | ACCGGGTGCAGCCCGAGATCCTGGAGGCCCTGCTGAAGCACATCTCCTTCGACAAGTTCGTGCAGATCTCCCTGAAGGCCCTG |
| | | CGGCGGATCGTGCCCCTGATGGAGCAGGGCAAGCGGTACGACGAGGCCTGCGCCGAGATCTACGGCGACCACTACGGCAAGAA |
| | | GAACACCGAGGAGAAGATCTACCTGCCCCCCATCCCCGCCGACGAGATCCGGAACCCCGTGGTGCTGCGGGCCCTGTCCCAGG |
| | | CCCGGAAGGTGATCAACGGCGTGGTGCGGCGGTACGGCTCCCCCGCCCGGATCCACATCGAGACCGCCCGGGAGGTGGGCAAG |
| | | TCCTTCAAGGACCGGAAGGAGATCGAGAAGCGGCAGGAGGAGAACCGGAAGGACCGGGAGAAGGCCGCCGCCAAGTTCCGGGA |
| | | GTACTTCCCCAACTTCGTGGGCGAGCCCAAGTCCAAGGACATCCTGAAGCTGCGGCTGTACGAGCAGCAGCACGGCAAGTGCC |
| | | TGTACTCCGGCAAGGAGATCAACCTGGTGCGGCTGAACGAGAAGGGCTACGTGGAGATCGACCACGCCCTGCCCTTCTCCCGG |
| | | ACCTGGGACGACTCCTTCAACAACAAGGTGCTGGTGCTGGGCTCCGAGAACCAGAACAAGGGCAACCAGACCCCCTACGAGTA |
| | | CTTCAACGGCAAGGACAACTCCCGGGAGTGGCAGGAGTTCAAGGCCCGGGTGGAGACCTCCCGGTTCCCCCGGTCCAAGAAGC |
| | | AGCGGATCCTGCTGCAGAAGTTCGACGAGGACGGCTTCAAGGAGTGCAACCTGAACGACACCCGGTACGTGAACCGGTTCCTG |
| | | TGCCAGTTCGTGGCCGACCACATCCTGCTGACCGGCAAGGGCAAGCGGCGGGTGTTCGCCTCCAACGGCCAGATCACCAACCT |
| | | GCTGCGGGGCTTCTGGGGCCTGCGGAAGGTGCGGGCCGAGAACGACCGGCACCACGCCCTGGACGCCGTGGTGGTGGCCTGCT |
| | | CCACCGTGGCCATGCAGCAGAAGATCACCCGGTTCGTGCGGTACAAGGAGATGAACGCCTTCGACGGCAAGACCATCGACAAG |
| | | GAGACCGGCAAGGTGCTGCACCAGAAGACCCACTTCCCCCAGCCCTGGGAGTTCTTCGCCCAGGAGGTGATGATCCGGGTGTT |
| | | CGGCAAGCCCGACGGCAAGCCCGAGTTCGAGGAGGCCGACACCCCCGAGAAGCTGCGGACCCTGCTGGCCGAGAAGCTGTCCT |
| | | CCCGGCCCGAGGCCGTGCACGAGTACGTGACCCCCCTGTTCGTGTCCCGGGCCCCCAACCGGAAGATGTCCGGCGCCCACAAG |
| | | GACACCCTGCGGTCCGCCAAGCGGTTCGTGAAGCACAACGAGAAGATCTCCGTGAAGCGGGTGTGGCTGACCGAGATCAAGCT |
| | | GGCCGACCTGGAGAACATGGTGAACTACAAGAACGGCCGGGAGATCGAGCTGTACGAGGCCCTGAAGGCCCGGCTGGAGGCCT |
| | | ACGGCGGCAACGCCAAGCAGGCCTTCGACCCCAAGGACAACCCCTTCTACAAGAAGGGCGGCCAGCTGGTGAAGGCCGTGCGG |
| | | GTGGAGAAGACCCAGGAGTCCGGCGTGCTGCTGAACAAGAAGAACGCCTACACCATCGCCGACAACGGCGACATGGTGCGGGT |
| | | GGACGTGTTCTGCAAGGTGGACAAGTCCGGCGGCGGCTCCCCCAAGAAGAAGCGGAAGGTGTCCGGCGGCTCCGGCAAGAACC |
| | | AGTACTTCATCGTGCCCATCTACGCCTGGCAGGTGGCCGAGAACATCCTGCCCGACATCGACTGCAAGGGCTACCGGATCGAC |
| | | GACTCCTACACCTTCTGCTTCTCCCTGCACAAGTACGACCTGATCGCCTTCCAGAAGGACGAGAAGTCCAAGGTGGAGTTCGC |
| | | CTACTACATCAACTGCGACTCCTCCAACGGCCGGTTCTACCTGGCCTGGCACGACAAGGGCTCCAAGGAGCAGCAGTTCCGGA |
| | | TCTCCACCCAGAACCTGGTGCTGATCCAGAAGTACCAGGTGAACGAGCTGGGCAAGGAGATCCGGCCCTGCCGGCTGAAGAAG |
| | | CGGCCCCCCGTGCGGTAGCTAGCaccagcctcaagaacacccgaatggagtctctaagctacataataccaacttacacttta |
| | | caaaatgttgtcccccaaaatgtagccattcgtatctgctcctaataaaaagaaagtttcttcacattctCTCGAGAAAAAAA |
| | | AAAAATGGAAAAAAAAAAAACGGAAAAAAAAAAAAGGTAAAAAAAAAAAATATAAAAAAAAAAAACATAAAAAAAAAAAACGA |
| | | AAAAAAAAAAACGTAAAAAAAAAAAACTCAAAAAAAAAAAAGATAAAAAAAAAAAACCTAAAAAAAAAAAATGTAAAAAAAAA |
| | | AAAGGGAAAAAAAAAAAACGCAAAAAAAAAAAACACAAAAAAAAAAAATGCAAAAAAAAAAAATCGAAAAAAAAAAAATCTAA |
| | | AAAAAAAAAACGAAAAAAAAAAAACCCAAAAAAAAAAAAGACAAAAAAAAAAAATAGAAAAAAAAAAAAGTTAAAAAAAAAAA |
| | | ACTGAAAAAAAAAAAATTTAAAAAAAAAAAAT |
| mRNA I | 19 | GGGaagctcagaataaacgctcaactttggccggatctgccacCATGGTGCCCAAGAAGAAGCGGAAGGTGGCCGCCTTCAAG |
| encoding | | CCCAACCCCATCAACTACATCCTGGGCCTGGACATCGGCATCGCCTCCGTGGGCTGGGCCATGGTGGAGATCGACGAGGAGGA |
| Nme2Cas9 | | GAACCCCATCCGGCTGATCGACCTGGGCGTGCGGGTGTTCGAGCGGGCCGAGGTGCCCAAGACCGGCGACTCCCTGGCCATGG |
| | | CCCGGCGGCTGGCCCGGTCCGTGCGGCGGCTGACCCGGCGGCGGGCCCACCGGCTGCTGCGGGCCCGGCGGCTGCTGAAGCGG |
| | | GAGGGCGTGCTGCAGGCCGCCGACTTCGACGAGAACGGCCTGATCAAGTCCCTGCCCAACACCCCCTGGCAGCTGCGGGCCGC |
| | | CGCCCTGGACCGGAAGCTGACCCCCCTGGAGTGGTCCGCCGTGCTGCTGCACCTGATCAAGCACCGGGGCTACCTGTCCCAGC |
| | | GGAAGAACGAGGGCGAGACCGCCGACAAGGAGCTGGGCGCCCTGCTGAAGGGCGTGGCCAACAACGCCCACGCCCTGCAGACC |
| | | GGCGACTTCCGGACCCCCGCCGAGCTGGCCCTGAACAAGTTCGAGAAGGAGTCCGGCCACATCCGGAACCAGCGGGGCGACTA |
| | | CTCCCACACCTTCTCCCGGAAGGACCTGCAGGCCGAGCTGATCCTGCTGTTCGAGAAGCAGAAGGAGTTCGGCAACCCCCACG |
| | | TGTCCGGCGGCCTGAAGGAGGGCATCGAGACCCTGCTGATGACCCAGCGGCCCGCCCTGTCCGGCGACGCCGTGCAGAAGATG |
| | | CTGGGCCACTGCACCTTCGAGCCCGCCGAGCCCAAGGCCGCCAAGAACACCTACACCGCCGAGCGGTTCATCTGGCTGACCAA |
| | | GCTGAACAACCTGCGGATCCTGGAGCAGGGCTCCGAGCGGCCCCTGACCGACACCGAGCGGGCCACCCTGATGGACGAGCCCT |
| | | ACCGGAAGTCCAAGCTGACCTACGCCCAGGCCCGGAAGCTGCTGGGCCTGGAGGACACCGCCTTCTTCAAGGGCCTGCGGTAC |
| | | GGCAAGGACAACGCCGAGGCCTCCACCCTGATGGAGATGAAGGCCTACCACGCCATCTCCCGGGCCCTGGAGAAGGAGGGCCT |
| | | GAAGGACAAGAAGTCCCCCCTGAACCTGTCCTCCGAGCTGCAGGACGAGATCGGCACCGCCTTCTCCCTGTTCAAGACCGACG |
| | | AGGACATCACCGGCCGGCTGAAGGACCGGGTGCAGCCCGAGATCCTGGAGGCCCTGCTGAAGCACATCTCCTTCGACAAGTTC |
| | | GTGCAGATCTCCCTGAAGGCCCTGCGGCGGATCGTGCCCCTGATGGAGCAGGGCAAGCGGTACGACGAGGCCTGCGCCGAGAT |
| | | CTACGGCGACCACTACGGCAAGAAGAACACCGAGGAGAAGATCTACCTGCCCCCCATCCCCGCCGACGAGATCCGGAACCCCG |
| | | TGGTGCTGCGGGCCCTGTCCCAGGCCCGGAAGGTGATCAACGGCGTGGTGCGGCGGTACGGCTCCCCCGCCCGGATCCACATC |
| | | GAGACCGCCCGGGAGGTGGGCAAGTCCTTCAAGGACCGGAAGGAGATCGAGAAGCGGCAGGAGGAGAACCGGAAGGACCGGGA |
| | | GAAGGCCGCCGCCAAGTTCCGGGAGTACTTCCCCAACTTCGTGGGCGAGCCCAAGTCCAAGGACATCCTGAAGCTGCGGCTGT |
| | | ACGAGCAGCAGCACGGCAAGTGCCTGTACTCCGGCAAGGAGATCAACCTGGTGCGGCTGAACGAGAAGGGCTACGTGGAGATC |
| | | GACCACGCCCTGCCCTTCTCCCGGACCTGGGACGACTCCTTCAACAACAAGGTGCTGGTGCTGGGCTCCGAGAACCAGAACAA |
| | | GGGCAACCAGACCCCCTACGAGTACTTCAACGGCAAGGACAACTCCCGGGAGTGGCAGGAGTTCAAGGCCCGGGTGGAGACCT |
| | | CCCGGTTCCCCCGGTCCAAGAAGCAGCGGATCCTGCTGCAGAAGTTCGACGAGGACGGCTTCAAGGAGTGCAACCTGAACGAC |
| | | ACCCGGTACGTGAACCGGTTCCTGTGCCAGTTCGTGGCCGACCACATCCTGCTGACCGGCAAGGGCAAGCGGCGGGTGTTCGC |
| | | CTCCAACGGCCAGATCACCAACCTGCTGCGGGGCTTCTGGGGCCTGCGGAAGGTGCGGGCCGAGAACGACCGGCACCACGCCC |
| | | TGGACGCCGTGGTGGTGGCCTGCTCCACCGTGGCCATGCAGCAGAAGATCACCCGGTTCGTGCGGTACAAGGAGATGAACGCC |
| | | TTCGACGGCAAGACCATCGACAAGGAGACCGGCAAGGTGCTGCACCAGAAGACCCACTTCCCCCAGCCCTGGGAGTTCTTCGC |
| | | CCAGGAGGTGATGATCCGGGTGTTCGGCAAGCCCGACGGCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGAGTACGTGACCCCCCTGTTCGTGTCCCGGGCCCCCAAC |
| | | CGGAAGATGTCCGGCGCCCACAAGGACACCCTGCGGTCCGCCAAGCGGTTCGTGAAGCACAACGAGAAGATCTCCGTGAAGCG |
| | | GGTGTGGCTGACCGAGATCAAGCTGGCCGACCTGGAGAACATGGTGAACTACAAGAACGGCCGGGAGATCGAGCTGTACGAGG |
| | | CCCTGAAGGCCCGGCTGGAGGCCTACGGCGGCAACGCCAAGCAGGCCTTCGACCCCAAGGACAACCCCTTCTACAAGAAGGGC |
| | | GGCCAGCTGGTGAAGGCCGTGCGGGTGGAGAAGACCCAGGAGTCCGGCGTGCTGCTGAACAAGAAGAACGCCTACACCATCGC |
| | | CGACAACGGCGACATGGTGCGGGTGGACGTGTTCTGCAAGGTGGACAAGAAGGGCAAGAACCAGTACTTCATCGTGCCCATCT |
| | | ACGCCTGGCAGGTGGCCGAGAACATCCTGCCCGACATCGACTGCAAGGGCTACCGGATCGACGACTCCTACACCTTCTGCTTC |
| | | TCCCTGCACAAGTACGACCTGATCGCCTTCCAGAAGGACGAGAAGTCCAAGGTGGAGTTCGCCTACTACATCAACTGCGACTC |
| | | CTCCAACGGCCGGTTCTACCTGGCCTGGCACGACAAGGGCTCCAAGGAGCAGCAGTTCCGGATCTCCACCCAGAACCTGGTGC |
| | | TGATCCAGAAGTACCAGGTGAACGAGCTGGGCAAGGAGATCCGGCCCTGCCGGCTGAAGAAGCGGCCCCCCGTGCGGTACCCC |
| | | TACGACGTGCCCGACTACGCCGCCGCCCCCGCCGCCAAGAAGAAGAAGCTGGACTAGCTAGCaccagcctcaagaacacccga |
| | | atggagtctctaagctacataataccaacttacactttacaaaatgttgtcccccaaaatgtagccattcgtatctgctccta |
| | | ataaaaagaaagtttcttcacattctCTCGAGAAAAAAAAAAAATGGAAAAAAAAAAAACGGAAAAAAAAAAAAGGTAAAAAA |
| | | AAAAAATATAAAAAAAAAAAACATAAAAAAAAAAAACGAAAAAAAAAAAACGTAAAAAAAAAAAACTCAAAAAAAAAAAAGAT |
| | | AAAAAAAAAAAACCTAAAAAAAAAAAATGTAAAAAAAAAAAAGGGAAAAAAAAAAAACGCAAAAAAAAAAAACACAAAAAAAA |
| | | AAAATGCAAAAAAAAAAAATCGAAAAAAAAAAAATCTAAAAAAAAAAAACGAAAAAAAAAAAACCCAAAAAAAAAAAAGACAA |
| | | AAAAAAAAAATAGAAAAAAAAAAAAGTTAAAAAAAAAAAACTGAAAAAAAAAAAATTTAAAAAAAAAAAAT |
| mRNA J | 20 | GGGAAGCUCAGAAUAAACGCUCAACUUUGGCCGGAUCUGCCACCAUGGUGCCCAAGAAGAAGCGGAAGGUGGAGGACAAGCGG |
| encoding | | CCCGCCGCCACCAAGAAGGCCGGCCAGGCCAAGAAGAAGAAGAUGGCCGCCUUCAAGCCCAACCCCAUCAACUACAUCCUGGG |
| Nme2Cas9 | | CCUGGACAUCGGCAUCGCCUCCGUGGGCUGGGCCAUGGUGGAGAUCGACGAGGAGGAGAACCCCAUCCGGCUGAUCGACCUGG |
| | | GCGUGCGGGUGUUCGAGCGGGCCGAGGUGCCCAAGACCGGCGACUCCCUGGCCAUGGCCCGGCGGCUGGCCCGGUCCGUGCGG |
| | | CGGCUGACCCGGCGGCGGGCCCACCGGCUGCUGCGGGCCCGGCGGCUGCUGAAGCGGGAGGGCGUGCUGCAGGCCGCCGACUU |
| | | CGACGAGAACGGCCUGAUCAAGUCCCUGCCCAACACCCCCUGGCAGCUGCGGGCCGCCGCCCUGGACCGGAAGCUGACCCCCC |
| | | UGGAGUGGUCCGCCGUGCUGCUGCACCUGAUCAAGCACCGGGGCUACCUGUCCCAGCGGAAGAACGAGGGCGAGACCGCCGAC |
| | | AAGGAGCUGGGCGCCCUGCUGAAGGGCGUGGCCAACAACGCCCACGCCCUGCAGACCGGCGACUUCCGGACCCCCGCCGAGCU |
| | | GGCCCUGAACAAGUUCGAGAAGGAGUCCGGCCACAUCCGGAACCAGCGGGGCGACUACUCCCACACCUUCUCCCGGAAGGACC |
| | | UGCAGGCCGAGCUGAUCCUGCUGUUCGAGAAGCAGAAGGAGUUCGGCAACCCCCACGUGUCCGGCGGCCUGAAGGAGGGCAUC |
| | | GAGACCCUGCUGAUGACCCAGCGGCCCGCCCUGUCCGGCGACGCCGUGCAGAAGAUGCUGGGCCACUGCACCUUCGAGCCCGC |
| | | CGAGCCCAAGGCCGCCAAGAACACCUACACCGCCGAGCGGUUCAUCUGGCUGACCAAGCUGAACAACCUGCGGAUCCUGGAGC |
| | | AGGGCUCCGAGCGGCCCCUGACCGACACCGAGCGGGCCACCCUGAUGGACGAGCCCUACCGGAAGUCCAAGCUGACCUACGCC |
| | | CAGGCCCGGAAGCUGCUGGGCCUGGAGGACACCGCCUUCUUCAAGGGCCUGCGGUACGGCAAGGACAACGCCGAGGCCUCCAC |
| | | CCUGAUGGAGAUGAAGGCCUACCACGCCAUCUCCCGGGCCCUGGAGAAGGAGGGCCUGAAGGACAAGAAGUCCCCCCUGAACC |
| | | UGUCCUCCGAGCUGCAGGACGAGAUCGGCACCGCCUUCUCCCUGUUCAAGACCGACGAGGACAUCACCGGCCGGCUGAAGGAC |
| | | CGGGUGCAGCCCGAGAUCCUGGAGGCCCUGCUGAAGCACAUCUCCUUCGACAAGUUCGUGCAGAUCUCCCUGAAGGCCCUGCG |
| | | GCGGAUCGUGCCCCUGAUGGAGCAGGGCAAGCGGUACGACGAGGCCUGCGCCGAGAUCUACGGCGACCACUACGGCAAGAAGA |
| | | ACACCGAGGAGAAGAUCUACCUGCCCCCCAUCCCCGCCGACGAGAUCCGGAACCCCGUGGUGCUGCGGGCCCUGUCCCAGGCC |
| | | CGGAAGGUGAUCAACGGCGUGGUGCGGCGGUACGGCUCCCCCGCCCGGAUCCACAUCGAGACCGCCCGGGAGGUGGGCAAGUC |
| | | CUUCAAGGACCGGAAGGAGAUCGAGAAGCGGCAGGAGGAGAACCGGAAGGACCGGGAGAAGGCCGCCGCCAAGUUCCGGGAGU |
| | | ACUUCCCCAACUUCGUGGGCGAGCCCAAGUCCAAGGACAUCCUGAAGCUGCGGCUGUACGAGCAGCAGCACGGCAAGUGCCUG |
| | | UACUCCGGCAAGGAGAUCAACCUGGUGCGGCUGAACGAGAAGGGCUACGUGGAGAUCGACCACGCCCUGCCCUUCUCCCGGAC |
| | | CUGGGACGACUCCUUCAACAACAAGGUGCUGGUGCUGGGCUCCGAGAACCAGAACAAGGGCAACCAGACCCCCUACGAGUACU |
| | | UCAACGGCAAGGACAACUCCCGGGAGUGGCAGGAGUUCAAGGCCCGGGUGGAGACCUCCCGGUUCCCCCGGUCCAAGAAGCAG |
| | | CGGAUCCUGCUGCAGAAGUUCGACGAGGACGGCUUCAAGGAGUGCAACCUGAACGACACCCGGUACGUGAACCGGUUCCUGUG |
| | | CCAGUUCGUGGCCGACCACAUCCUGCUGACCGGCAAGGGCAAGCGGCGGGUGUUCGCCUCCAACGGCCAGAUCACCAACCUGC |
| | | UGCGGGGCUUCUGGGGCCUGCGGAAGGUGCGGGCCGAGAACGACCGGCACCACGCCCUGGACGCCGUGGUGGUGGCCUGCUCC |
| | | ACCGUGGCCAUGCAGCAGAAGAUCACCCGGUUCGUGCGGUACAAGGAGAUGAACGCCUUCGACGGCAAGACCAUCGACAAGGA |
| | | GACCGGCAAGGUGCUGCACCAGAAGACCCACUUCCCCCAGCCCUGGGAGUUCUUCGCCCAGGAGGUGAUGAUCCGGGUGUUCG |
| | | GCAAGCCCGACGGCAAGCCCGAGUUCGAGGAGGCCGACACCCCCGAGAAGCUGCGGACCCUGCUGGCCGAGAAGCUGUCCUCC |
| | | CGGCCCGAGGCCGUGCACGAGUACGUGACCCCCCUGUUCGUGUCCCGGGCCCCCAACCGGAAGAUGUCCGGCGCCCACAAGGA |
| | | CACCCUGCGGUCCGCCAAGCGGUUCGUGAAGCACAACGAGAAGAUCUCCGUGAAGCGGGUGUGGCUGACCGAGAUCAAGCUGG |
| | | CCGACCUGGAGAACAUGGUGAACUACAAGAACGGCCGGGAGAUCGAGCUGUACGAGGCCCUGAAGGCCCGGCUGGAGGCCUAC |
| | | GGCGGCAACGCCAAGCAGGCCUUCGACCCCAAGGACAACCCCUUCUACAAGAAGGGCGGCCAGCUGGUGAAGGCCGUGCGGGU |
| | | GGAGAAGACCCAGGAGUCCGGCGUGCUGCUGAACAAGAAGAACGCCUACACCAUCGCCGACAACGGCGACAUGGUGCGGGUGG |
| | | ACGUGUUCUGCAAGGUGGACAAGAAGGGCAAGAACCAGUACUUCAUCGUGCCCAUCUACGCCUGGCAGGUGGCCGAGAACAUC |
| | | CUGCCCGACAUCGACUGCAAGGGCUACCGGAUCGACGACUCCUACACCUUCUGCUUCUCCCUGCACAAGUACGACCUGAUCGC |
| | | CUUCCAGAAGGACGAGAAGUCCAAGGUGGAGUUCGCCUACUACAUCAACUGCGACUCCUCCAACGGCCGGUUCUACCUGGCCU |
| | | GGCACGACAAGGGCUCCAAGGAGCAGCAGUUCCGGAUCUCCACCCAGAACCUGGUGCUGAUCCAGAAGUACCAGGUGAACGAG |
| | | CUGGGCAAGGAGAUCCGGCCCUGCCGGCUGAAGAAGCGGCCCCCCGUGCGGGAGGACAAGCGGCCCGCCGCCACCAAGAAGGC |
| | | CGGCCAGGCCAAGAAGAAGAAGUACCCCUACGACGUGCCCGACUACGCCGGCUACCCCUACGACGUGCCCGACUACGCCGGCU |
| | | CCUACCCCUACGACGUGCCCGACUACGCCGCCGCCCCCGCCGCCAAGAAGAAGAAGCUGGACUAGCUAGCACCAGCCUCAAGA |
| | | ACACCCGAAUGGAGUCUCUAAGCUACAUAAUACCAACUUACACUUUACAAAAUGUUGUCCCCCAAAAUGUAGCCAUUCGUAUC |
| | | UGCUCCUAAUAAAAAGAAAGUUUCUUCACAUUCUCUCGAGAAAAAAAAAAAAUGGAAAAAAAAAAAACGGAAAAAAAAAAAAG |
| | | GUAAAAAAAAAAAAUAUAAAAAAAAAAAACAUAAAAAAAAAAAACGAAAAAAAAAAAACGUAAAAAAAAAAAACUCAAAAAAA |
| | | AAAAAGAUAAAAAAAAAAAACCUAAAAAAAAAAAAUGUAAAAAAAAAAAAGGGAAAAAAAAAAAACGCAAAAAAAAAAAACAC |
| | | AAAAAAAAAAAAUGCAAAAAAAAAAAAUCGAAAAAAAAAAAAUCUAAAAAAAAAAAACGAAAAAAAAAAAACCCAAAAAAAAA |
| | | AAAGACAAAAAAAAAAAAUAGAAAAAAAAAAAAGUUAAAAAAAAAAAACUGAAAAAAAAAAAAUUUAAAAAAAAAAAAUCUAG |
| mRNA K | 21 | GGGaagctcagaataaacgctcaactttggccggatctgccacCatggccgccttcaagcccaaccccatcaactacatcctg |
| encoding | | ggcctggacatcggcatcgcctccgtgggctgggccatggtggagatcgacgaggaggagaaccccatccggctgatcgacct |
| Nme2Cas9 | | gggcgtgcgggtgttcgagcgggccgaggtgcccaagaccggcgactccctggccatggcccggcggctggcccggtccgtgc |
| | | ggcggctgacccggcggcgggcccaccggctgctgcgggcccggcggctgctgaagcgggagggcgtgctgcaggccgccgac |
| | | ttcgacgagaacggcctgatcaagtccctgcccaacaccccctggcagctgcgggccgccgccctggaccggaagctgacccc |
| | | cctggagtggtccgccgtgctgctgcacctgatcaagcaccggggctacctgtcccagcggaagaacgagggcgagaccgccg |
| | | acaaggagctgggcgccctgctgaagggcgtggccaacaacgcccacgccctgcagaccggcgacttccggacccccgccgag |
| | | ctggccctgaacaagttcgagaaggagtccggccacatccggaaccagcggggcgactactcccacaccttctcccggaagga |
| | | cctgcaggccgagctgatcctgctgttcgagaagcagaaggagttcggcaacccccacgtgtccggcggcctgaaggagggca |
| | | tcgagaccctgctgatgacccagcggcccgccctgtccggcgacgccgtgcagaagatgctgggccactgcaccttcgagccc |
| | | gccgagcccaaggccgccaagaacacctacaccgccgagcggttcatctggctgaccaagctgaacaacctgcggatcctgga |
| | | gcagggctccgagcggcccctgaccgacaccgagcgggccaccctgatggacgagccctaccggaagtccaagctgacctacg |
| | | cccaggcccggaagctgctgggcctggaggacaccgccttcttcaagggcctgcggtacggcaaggacaacgccgaggcctcc |
| | | accctgatggagatgaaggcctaccacgccatctcccgggccctggagaaggagggcctgaaggacaagaagtcccccctgaa |
| | | cctgtcctccgagctgcaggacgagatcggcaccgccttctccctgttcaagaccgacgaggacatcaccggccggctgaagg |
| | | accgggtgcagcccgagatcctggaggccctgctgaagcacatctccttcgacaagttcgtgcagatctccctgaaggccctg |
| | | cggcggatcgtgcccctgatggagcagggcaagcggtacgacgaggcctgcgccgagatctacggcgaccactacggcaagaa |
| | | gaacaccgaggagaagatctacctgccccccatccccgccgacgagatccggaaccccgtggtgctgcgggccctgtcccagg |
| | | cccggaaggtgatcaacggcgtggtgcggcggtacggctcccccgcccggatccacatcgagaccgcccgggaggtgggcaag |
| | | tccttcaaggaccggaaggagatcgagaagcggcaggaggagaaccggaaggaccgggagaaggccgccgccaagttccggga |
| | | gtacttccccaacttcgtgggcgagcccaagtccaaggacatcctgaagctgcggctgtacgagcagcagcacggcaagtgcc |
| | | tgtactccggcaaggagatcaacctggtgcggctgaacgagaagggctacgtggagatcgaccacgccctgcccttctcccgg |
| | | acctgggacgactccttcaacaacaaggtgctggtgctgggctccgagaaccagaacaagggcaaccagaccccctacgagta |
| | | cttcaacggcaaggacaactcccgggagtggcaggagttcaaggcccgggtggagacctcccggttcccccggtccaagaagc |
| | | agcggatcctgctgcagaagttcgacgaggacggcttcaaggagtgcaacctgaacgacacccggtacgtgaaccggttcctg |
| | | tgccagttcgtggccgaccacatcctgctgaccggcaagggcaagcggcgggtgttcgcctccaacggccagatcaccaacct |
| | | gctgcggggcttctggggcctgcggaaggtgcgggccgagaacgaccggcaccacgccctggacgccgtggtggtggcctgct |
| | | ccaccgtggccatgcagcagaagatcacccggttcgtgcggtacaaggagatgaacgccttcgacggcaagaccatcgacaag |
| | | gagaccggcaaggtgctgcaccagaagacccacttcccccagccctgggagttcttcgcccaggaggtgatgatccgggtgtt |
| | | cggcaagcccgacggcaagcccgagttcgaggaggccgacacccccgagaagctgcggaccctgctggccgagaagctgtcct |
| | | cccggcccgaggccgtgcacgagtacgtgacccccctgttcgtgtcccgggcccccaaccggaagatgtccggcgcccacaag |
| | | gacaccctgcggtccgccaagcggttcgtgaagcacaacgagaagatctccgtgaagcgggtgtggctgaccgagatcaagct |
| | | ggccgacctggagaacatggtgaactacaagaacggccgggagatcgagctgtacgaggccctgaaggcccggctggaggcct |
| | | acggcggcaacgccaagcaggccttcgaccccaaggacaaccccttctacaagaagggcggccagctggtgaaggccgtgcgg |
| | | gtggagaagacccaggagtccggcgtgctgctgaacaagaagaacgcctacaccatcgccgacaacggcgacatggtgcgggt |
| | | ggacgtgttctgcaaggtggacaagaagggcaagaaccagtacttcatcgtgcccatctacgcctggcaggtggccgagaaca |
| | | tcctgcccgacatcgactgcaagggctaccggatcgacgactcctacaccttctgcttctccctgcacaagtacgacctgatc |
| | | gccttccagaaggacgagaagtccaaggtggagttcgcctactacatcaactgcgactcctccaacggccggttctacctggc |
| | | ctggcacgacaagggctccaaggagcagcagttccggatctccacccagaacctggtgctgatccagaagtaccaggtgaacg |
| | | agctgggcaaggagatccggccctgccggctgaagaagcggccccccgtgcggTCCGGAAAGCGGACCGCCGACGGCTCCGGA |
| | | GGAGGAAGCCCCAAGAAGAAGCGGAAGGTGtagctagcaccagcctcaagaacacccgaatggagtctctaagctacataata |
| | | ccaacttacactttacaaaatgttgtcccccaaaatgtagccattcgtatctgctcctaataaaaagaaagtttcttcacatt |
| | | ctctcgagAAAAAAAAAAAATGGAAAAAAAAAAAACGGAAAAAAAAAAAAGGTAAAAAAAAAAAATATAAAAAAAAAAAACAT |
| | | AAAAAAAAAAAACGAAAAAAAAAAAACGTAAAAAAAAAAACTCAAAAAAAAAAAGATAAAAAAAAAAAACCTAAAAAAAAAAA |
| | | ATGTAAAAAAAAAAAAGGGAAAAAAAAAAAACGCAAAAAAAAAAAACACAAAAAAAAAAAATGCAAAAAAAAAAAATCGAAAA |
| | | AAAAAAAATCTAAAAAAAAAAAACGAAAAAAAAAAAACCCAAAAAAAAAAAAGACAAAAAAAAAAAATAGAAAAAAAAAAAGT |
| | | TAAAAAAAAAAAACTGAAAAAAAAAAAATTTAAAAAAAAAAAAT |
| mRNA L | 22 | GGGaagctcagaataaacgctcaactttggccggatctgccacCatgGACGGCTCCGGCGGCGGCTCCCCCAAGAAGAAGCGG |
| encoding | | AAGGTGGGCGGCTCCGGCGGCGGCgccgccttcaagcccaaccccatcaactacatcctgggcctggacatcggcatcgcctc |
| Nme2Cas9 | | cgtgggctgggccatggtggagatcgacgaggaggagaaccccatccggctgatcgacctgggcgtgcgggtgttcgagcggg |
| | | ccgaggtgcccaagaccggcgactccctggccatggcccggcggctggcccggtccgtgcggcggctgacccggcggcgggcc |
| | | caccggctgctgcgggcccggcggctgctgaagcgggagggcgtgctgcaggccgccgacttcgacgagaacggcctgatcaa |
| | | gtccctgcccaacaccccctggcagctgcgggccgccgccctggaccggaagctgacccccctggagtggtccgccgtgctgc |
| | | tgcacctgatcaagcaccggggctacctgtcccagcggaagaacgagggcgagaccgccgacaaggagctgggcgccctgctg |
| | | aagggcgtggccaacaacgcccacgccctgcagaccggcgacttccggacccccgccgagctggccctgaacaagttcgagaa |
| | | ggagtccggccacatccggaaccagcggggcgactactcccacaccttctcccggaaggacctgcaggccgagctgatcctgc |
| | | tgttcgagaagcagaaggagttcggcaacccccacgtgtccggcggcctgaaggagggcatcgagaccctgctgatgacccag |
| | | cggcccgccctgtccggcgacgccgtgcagaagatgctgggccactgcaccttcgagcccgccgagcccaaggccgccaagaa |
| | | cacctacaccgccgagcggttcatctggctgaccaagctgaacaacctgcggatcctggagcagggctccgagcggcccctga |
| | | ccgacaccgagcgggccaccctgatggacgagccctaccggaagtccaagctgacctacgcccaggcccggaagctgctgggc |
| | | ctggaggacaccgccttcttcaagggcctgcggtacggcaaggacaacgccgaggcctccaccctgatggagatgaaggccta |
| | | ccacgccatctcccgggccctggagaaggagggcctgaaggacaagaagtcccccctgaacctgtcctccgagctgcaggacg |
| | | agatcggcaccgccttctccctgttcaagaccgacgaggacatcaccggccggctgaaggaccgggtgcagcccgagatcctg |
| | | gaggccctgctgaagcacatctccttcgacaagttcgtgcagatctccctgaaggccctgcggcggatcgtgcccctgatgga |
| | | gcagggcaagcggtacgacgaggcctgcgccgagatctacggcgaccactacggcaagaagaacaccgaggagaagatctacc |
| | | tgccccccatccccgccgacgagatccggaaccccgtggtgctgcgggccctgtcccaggcccggaaggtgatcaacggcgtg |
| | | gtgcggcggtacggctcccccgcccggatccacatcgagaccgcccgggaggtgggcaagtccttcaaggaccggaaggagat |
| | | cgagaagcggcaggaggagaaccggaaggaccgggagaaggccgccgccaagttccgggagtacttccccaacttcgtgggcg |
| | | agcccaagtccaaggacatcctgaagctgcggctgtacgagcagcagcacggcaagtgcctgtactccggcaaggagatcaac |
| | | ctggtgcggctgaacgagaagggctacgtggagatcgaccacgccctgcccttctcccggacctgggacgactccttcaacaa |
| | | caaggtgctggtgctgggctccgagaaccagaacaagggcaaccagaccccctacgagtacttcaacggcaaggacaactccc |
| | | gggagtggcaggagttcaaggcccgggtggagacctcccggttcccccggtccaagaagcagcggatcctgctgcagaagttc |
| | | gacgaggacggcttcaaggagtgcaacctgaacgacacccggtacgtgaaccggttcctgtgccagttcgtggccgaccacat |
| | | cctgctgaccggcaagggcaagcggcgggtgttcgcctccaacggccagatcaccaacctgctgcggggcttctggggcctgc |
| | | ggaaggtgcgggccgagaacgaccggcaccacgccctggacgccgtggtggtggcctgctccaccgtggccatgcagcagaag |
| | | atcacccggttcgtgcggtacaaggagatgaacgccttcgacggcaagaccatcgacaaggagaccggcaaggtgctgcacca |
| | | gaagacccacttcccccagccctgggagttcttcgcccaggaggtgatgatccgggtgttcggcaagcccgacggcaagcccg |
| | | agttcgaggaggccgacacccccgagaagctgcggaccctgctggccgagaagctgtcctcccggcccgaggccgtgcacgag |
| | | tacgtgacccccctgttcgtgtcccgggcccccaaccggaagatgtccggcgcccacaaggacaccctgcggtccgccaagcg |
| | | gttcgtgaagcacaacgagaagatctccgtgaagcgggtgtggctgaccgagatcaagctggccgacctggagaacatggtga |
| | | actacaagaacggccgggagatcgagctgtacgaggccctgaaggcccggctggaggcctacggcggcaacgccaagcaggcc |
| | | ttcgaccccaaggacaaccccttctacaagaagggcggccagctggtgaaggccgtgcgggtggagaagacccaggagtccgg |
| | | cgtgctgctgaacaagaagaacgcctacaccatcgccgacaacggcgacatggtgcgggtggacgtgttctgcaaggtggaca |
| | | agaagggcaagaaccagtacttcatcgtgcccatctacgcctggcaggtggccgagaacatcctgcccgacatcgactgcaag |
| | | ggctaccggatcgacgactcctacaccttctgcttctccctgcacaagtacgacctgatcgccttccagaaggacgagaagtc |
| | | caaggtggagttcgcctactacatcaactgcgactcctccaacggccggttctacctggcctggcacgacaagggctccaagg |
| | | agcagcagttccggatctccacccagaacctggtgctgatccagaagtaccaggtgaacgagctgggcaaggagatccggccc |
| | | tgccggctgaagaagcggccccccgtgcggtagctagcaccagcctcaagaacacccgaatggagtctctaagctacataata |
| | | ccaacttacactttacaaaatgttgtcccccaaaatgtagccattcgtatctgctcctaataaaaagaaagtttcttcacatt |
| | | ctctcgagAAAAAAAAAAAATGGAAAAAAAAAAAACGGAAAAAAAAAAAAGGTAAAAAAAAAAAATATAAAAAAAAAAAACAT |
| | | AAAAAAAAAAAACGAAAAAAAAAAAACGTAAAAAAAAAAAACTCAAAAAAAAAAAGATAAAAAAAAAAAACCTAAAAAAAAAA |
| | | AATGTAAAAAAAAAAAAGGGAAAAAAAAAAAACGCAAAAAAAAAAAACACAAAAAAAAAAAATGCAAAAAAAAAAAATCGAAA |
| | | AAAAAAAAATCTAAAAAAAAAAAACGAAAAAAAAAAAACCCAAAAAAAAAAAAGACAAAAAAAAAAAATAGAAAAAAAAAAAG |
| | | TTAAAAAAAAAAAACTGAAAAAAAAAAAATTTAAAAAAAAAAAAT |
| mRNA M | 23 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGGCGGCUCCGGCGGCGGCGCCGCCUUCAAGCCCAACCCCAUCAACUACAUCCUGGGCCUGGACAUCGGCAUCGCCUC |
| Nme2Cas9 | | CGUGGGCUGGGCCAUGGUGGAGAUCGACGAGGAGGAGAACCCCAUCCGGCUGAUCGACCUGGGCGUGCGGGUGUUCGAGcGGG |
| with | | CCGAGGUGCCCAAGACCGGCGACUCCCUGGCCAUGGCCCGGCGGCUGGCCCGGUCCGUGCGGCGGCUGACCCGGCGGCGGGCC |
| HiBiT tag | | CACCGGCUGCUGCGGGCCCGGCGGCUGCUGAAGCGGGAGGGCGUGCUGCAGGCCGCCGACUUCGACGAGAACGGCCUGAUCAA |
| | | GUCCCUGCCCAACACCCCCUGGCAGCUGCGGGCCGCCGCCCUGGACCGGAAGCUGACCCCCCUGGAGUGGUCCGCCGUGCUGC |
| | | UGCACCUGAUCAAGCACCGGGGCUACCUGUCCCAGCGGAAGAACGAGGGCGAGACCGCCGACAAGGAGCUGGGCGCCCUGCUG |
| | | AAGGGCGUGGCCAACAACGCCCACGCCCUGCAGACCGGCGACUUCCGGACCCCCGCCGAGCUGGCCCUGAACAAGUUCGAGAA |
| | | GGAGUCCGGCCACAUCCGGAACCAGCGGGGCGACUACUCCCACACCUUCUCCCGGAAGGACCUGCAGGCCGAGCUGAUCCUGC |
| | | UGUUCGAGAAGCAGAAGGAGUUCGGCAACCCCCACGUGUCCGGCGGCCUGAAGGAGGGCAUCGAGACCCUGCUGAUGACCCAG |
| | | CGGCCCGCCCUGUCCGGCGACGCCGUGCAGAAGAUGCUGGGCCACUGCACCUUCGAGCCCGCCGAGCCCAAGGCCGCCAAGAA |
| | | CACCUACACCGCCGAGCGGUUCAUCUGGCUGACCAAGCUGAACAACCUGCGGAUCCUGGAGCAGGGCUCCGAGCGGCCCCUGA |
| | | CCGACACCGAGCGGGCCACCCUGAUGGACGAGCCCUACCGGAAGUCCAAGCUGACCUACGCCCAGGCCCGGAAGCUGCUGGGC |
| | | CUGGAGGACACCGCCUUCUUCAAGGGCCUGCGGUACGGCAAGGACAACGCCGAGGCCUCCACCCUGAUGGAGAUGAAGGCCUA |
| | | CCACGCCAUCUCCCGGGCCCUGGAGAAGGAGGGCCUGAAGGACAAGAAGUCCCCCCUGAACCUGUCCUCCGAGCUGCAGGACG |
| | | AGAUCGGCACCGCCUUCUCCCUGUUCAAGACCGACGAGGACAUCACCGGCCGGCUGAAGGACCGGGUGCAGCCCGAGAUCCUG |
| | | GAGGCCCUGCUGAAGCACAUCUCCUUCGACAAGUUCGUGCAGAUCUCCCUGAAGGCCCUGCGGCGGAUCGUGCCCCUGAUGGA |
| | | GCAGGGCAAGCGGUACGACGAGGCCUGCGCCGAGAUCUACGGCGACCACUACGGCAAGAAGAACACCGAGGAGAAGAUCUACC |
| | | UGCCCCCCAUCCCCGCCGACGAGAUCCGGAACCCCGUGGUGCUGCGGGCCCUGUCCCAGGCCCGGAAGGUGAUCAACGGCGUG |
| | | GUGCGGCGGUACGGCUCCCCCGCCCGGAUCCACAUCGAGACCGCCCGGGAGGUGGGCAAGUCCUUCAAGGACCGGAAGGAGAU |
| | | CGAGAAGCGGCAGGAGGAGAACCGGAAGGACCGGGAGAAGGCCGCCGCCAAGUUCCGGGAGUACUUCCCCAACUUCGUGGGCG |
| | | AGCCCAAGUCCAAGGACAUCCUGAAGCUGCGGCUGUACGAGCAGCAGCACGGCAAGUGCCUGUACUCCGGCAAGGAGAUCAAC |
| | | CUGGUGCGGCUGAACGAGAAGGGCUACGUGGAGAUCGACCACGCCCUGCCCUUCUCCCGGACCUGGGACGACUCCUUCAACAA |
| | | CAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUC |
| | | GACGAGGACGGCUUCAAGGAGUGCAACCUGAACGACACCCGGUACGUGAACCGGUUCCUGUGCCAGUUCGUGGCCGACCACAU |
| | | CCUGCUGACCGGCAAGGGCAAGCGGCGGGUGUUCGCCUCCAACGGCCAGAUCACCAACCUGCUGCGGGGCUUCUGGGGCCUGC |
| | | GGAAGGUGCGGGCCGAGAACGACCGGCACCACGCCCUGGACGCCGUGGUGGUGGCCUGCUCCACCGUGGCCAUGCAGCAGAAG |
| | | AUCACCCGGUUCGUGCGGUACAAGGAGAUGAACGCCUUCGACGGCAAGACCAUCGACAAGGAGACCGGCAAGGUGCUGCACCA |
| | | GAAGACCCACUUCCCCCAGCCCUGGGAGUUCUUCGCCCAGGAGGUGAUGAUCCGGGUGUUCGGCAAGCCCGACGGCAAGCCCG |
| | | AGUUCGAGGAGGCCGACACCCCCGAGAAGCUGCGGACCCUGCUGGCCGAGAAGCUGUCCUCCCGGCCCGAGGCCGUGCACGAG |
| | | UACGUGACCCCCCUGUUCGUGUCCCGGGCCCCCAACCGGAAGAUGUCCGGCGCCCACAAGGACACCCUGCGGUCCGCCAAGCG |
| | | GUUCGUGAAGCACAACGAGAAGAUCUCCGUGAAGCGGGUGUGGCUGACCGAGAUCAAGCUGGCCGACCUGGAGAACAUGGUGA |
| | | ACUACAAGAACGGCCGGGAGAUCGAGCUGUACGAGGCCCUGAAGGCCCGGCUGGAGGCCUACGGCGGCAACGCCAAGCAGGCC |
| | | UUCGACCCCAAGGACAACCCCUUCUACAAGAAGGGCGGCCAGCUGGUGAAGGCCGUGCGGGUGGAGAAGACCCAGGAGUCCGG |
| | | CGUGCUGCUGAACAAGAAGAACGCCUACACCAUCGCCGACAACGGCGACAUGGUGCGGGUGGACGUGUUCUGCAAGGUGGACA |
| | | AGAAGGGCAAGAACCAGUACUUCAUCGUGCCCAUCUACGCCUGGCAGGUGGCCGAGAACAUCCUGCCCGACAUCGACUGCAAG |
| | | GGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGG |
| | | AGCAGCAGUUCCGGAUCUCCACCCAGAACCUGGUGCUGAUCCAGAAGUACCAGGUGAACGAGCUGGGCAAGGAGAUCCGGCCC |
| | | UGCCGGCUGAAGAAGCGGCCCCCCGUGCGGUCCGAGUCCGCCACCCCCGAGUCCGUGUCCGGCUGGCGGCUGUUCAAGAAGAU |
| | | CUCCUAGCUAGCACCAGCCUCAAGAACACCCGAAUGGAGUCUCUAAGCUACAUAAUACCAACUUACACUUUACAAAAUGUUGU |
| | | CCCCCAAAAUGUAGCCAUUCGUAUCUGCUCCUAAUAAAAAGAAAGUUUCUUCACAUUCUCUCGAGAAAAAAAAAAAAUGGAAA |
| | | AAAAAAAAACGGAAAAAAAAAAAAGGUAAAAAAAAAAAAUAUAAAAAAAAAAAACAUAAAAAAAAAAAACGAAAAAAAAAAAA |
| | | CGUAAAAAAAAAAAACUCAAAAAAAAAAAAGAUAAAAAAAAAAAACCUAAAAAAAAAAAAUGUAAAAAAAAAAAAGGGAAAAA |
| | | AAAAAAACGCAAAAAAAAAAAACACAAAAAAAAAAAAUGCAAAAAAAAAAAAUCGAAAAAAAAAAAAUCUAAAAAAAAAAAAC |
| | | GAAAAAAAAAAAACCCAAAAAAAAAAAAGACAAAAAAAAAAAAUAGAAAAAAAAAAAAGUUAAAAAAAAAAAACUGAAAAAAA |
| | | AAAAAUUUAAAAAAAAAAAAUCUAG |
| mRNA N | 24 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGGCGGCUCCGGCGGCGGCGCCGCCUUCAAGCCCAACCCCAUCAACUACAUCCUGGGCCUGGACAUCGGCAUCGCCUC |
| Nme2Cas9 | | CGUGGGCUGGGCCAUGGUGGAGAUCGACGAGGAGGAGAACCCCAUCCGGCUGAUCGACCUGGGCGUGCGGGUGUUCGAGCGGG |
| | | CCGAGGUGCCCAAGACCGGCGACUCCCUGGCCAUGGCCCGGCGGCUGGCCCGGUCCGUGCGGCGGCUGACCCGGCGGCGGGCC |
| | | CACCGGCUGCUGCGGGCCCGGCGGCUGCUGAAGCGGGAGGGCGUGCUGCAGGCCGCCGACUUCGACGAGAACGGCCUGAUCAA |
| | | GUCCCUGCCCAACACCCCCUGGCAGCUGCGGGCCGCCGCCCUGGACCGGAAGCUGACCCCCCUGGAGUGGUCCGCCGUGCUGC |
| | | UGCACCUGAUCAAGCACCGGGGCUACCUGUCCCAGCGGAAGAACGAGGGCGAGACCGCCGACAAGGAGCUGGGCGCCCUGCUG |
| | | AAGGGCGUGGCCAACAACGCCCACGCCCUGCAGACCGGCGACUUCCGGACCCCCGCCGAGCUGGCCCUGAACAAGUUCGAGAA |
| | | GGAGUCCGGCCACAUCCGGAACCAGCGGGGCGACUACUCCCACACCUUCUCCCGGAAGGACCUGCAGGCCGAGCUGAUCCUGC |
| | | UGUUCGAGAAGCAGAAGGAGUUCGGCAACCCCCACGUGUCCGGCGGCCUGAAGGAGGGCAUCGAGACCCUGCUGAUGACCCAG |
| | | CGGCCCGCCCUGUCCGGCGACGCCGUGCAGAAGAUGCUGGGCCACUGCACCUUCGAGCCCGCCGAGCCCAAGGCCGCCAAGAA |
| | | CACCUACACCGCCGAGCGGUUCAUCUGGCUGACCAAGCUGAACAACCUGCGGAUCCUGGAGCAGGGCUCCGAGCGGCCCCUGA |
| | | CCGACACCGAGCGGGCCACCCUGAUGGACGAGCCCUACCGGAAGUCCAAGCUGACCUACGCCCAGGCCCGGAAGCUGCUGGGC |
| | | CUGGAGGACACCGCCUUCUUCAAGGGCCUGCGGUACGGCAAGGACAACGCCGAGGCCUCCACCCUGAUGGAGAUGAAGGCCUA |
| | | CCACGCCAUCUCCCGGGCCCUGGAGAAGGAGGGCCUGAAGGACAAGAAGUCCCCCCUGAACCUGUCCUCCGAGCUGCAGGACG |
| | | AGAUCGGCACCGCCUUCUCCCUGUUCAAGACCGACGAGGACAUCACCGGCCGGCUGAAGGACCGGGUGCAGCCCGAGAUCCUG |
| | | GAGGCCCUGCUGAAGCACAUCUCCUUCGACAAGUUCGUGCAGAUCUCCCUGAAGGCCCUGCGGCGGAUCGUGCCCCUGAUGGA |
| | | GCAGGGCAAGCGGUACGACGAGGCCUGCGCCGAGAUCUACGGCGACCACUACGGCAAGAAGAACACCGAGGAGAAGAUCUACC |
| | | UGCCCCCCAUCCCCGCCGACGAGAUCCGGAACCCCGUGGUGCUGCGGGCCCUGUCCCAGGCCCGGAAGGUGAUCAACGGCGUG |
| | | GUGCGGCGGUACGGCUCCCCCGCCCGGAUCCACAUCGAGACCGCCCGGGAGGUGGGCAAGUCCUUCAAGGACCGGAAGGAGAU |
| | | CGAGAAGCGGCAGGAGGAGAACCGGAAGGACCGGGAGAAGGCCGCCGCCAAGUUCCGGGAGUACUUCCCCAACUUCGUGGGCG |
| | | AGCCCAAGUCCAAGGACAUCCUGAAGCUGCGGCUGUACGAGCAGCAGCACGGCAAGUGCCUGUACUCCGGCAAGGAGAUCAAC |
| | | CUGGUGCGGCUGAACGAGAAGGGCUACGUGGAGAUCGACCACGCCCUGCCCUUCUCCCGGACCUGGGACGACUCCUUCAACAA |
| | | CAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUC |
| | | GACGAGGACGGCUUCAAGGAGUGCAACCUGAACGACACCCGGUACGUGAACCGGUUCCUGUGCCAGUUCGUGGCCGACCACAU |
| | | CCUGCUGACCGGCAAGGGCAAGCGGCGGGUGUUCGCCUCCAACGGCCAGAUCACCAACCUGCUGCGGGGCUUCUGGGGCCUGC |
| | | GGAAGGUGCGGGCCGAGAACGACCGGCACCACGCCCUGGACGCCGUGGUGGUGGCCUGCUCCACCGUGGCCAUGCAGCAGAAG |
| | | AUCACCCGGUUCGUGCGGUACAAGGAGAUGAACGCCUUCGACGGCAAGACCAUCGACAAGGAGACCGGCAAGGUGCUGCACCA |
| | | GAAGACCCACUUCCCCCAGCCCUGGGAGUUCUUCGCCCAGGAGGUGAUGAUCCGGGUGUUCGGCAAGCCCGACGGCAAGCCCG |
| | | AGUUCGAGGAGGCCGACACCCCCGAGAAGCUGCGGACCCUGCUGGCCGAGAAGCUGUCCUCCCGGCCCGAGGCCGUGCACGAG |
| | | UACGUGACCCCCCUGUUCGUGUCCCGGGCCCCCAACCGGAAGAUGUCCGGCGCCCACAAGGACACCCUGCGGUCCGCCAAGCG |
| | | GUUCGUGAAGCACAACGAGAAGAUCUCCGUGAAGCGGGUGUGGCUGACCGAGAUCAAGCUGGCCGACCUGGAGAACAUGGUGA |
| | | ACUACAAGAACGGCCGGGAGAUCGAGCUGUACGAGGCCCUGAAGGCCCGGCUGGAGGCCUACGGCGGCAACGCCAAGCAGGCC |
| | | UUCGACCCCAAGGACAACCCCUUCUACAAGAAGGGCGGCCAGCUGGUGAAGGCCGUGCGGGUGGAGAAGACCCAGGAGUCCGG |
| | | CGUGCUGCUGAACAAGAAGAACGCCUACACCAUCGCCGACAACGGCGACAUGGUGCGGGUGGACGUGUUCUGCAAGGUGGACA |
| | | AGAAGGGCAAGAACCAGUACUUCAUCGUGCCCAUCUACGCCUGGCAGGUGGCCGAGAACAUCCUGCCCGACAUCGACUGCAAG |
| | | GGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGG |
| | | AGCAGCAGUUCCGGAUCUCCACCCAGAACCUGGUGCUGAUCCAGAAGUACCAGGUGAACGAGCUGGGCAAGGAGAUCCGGCCC |
| | | UGCCGGCUGAAGAAGCGGCCCCCCGUGCGGUCCGGAAAGCGGACCGCCGACGGCUCCGGAGGAGGAAGCCCCGCCGCCAAGAA |
| | | GAAGAAGCUGGACUAGCUAGCACCAGCCUCAAGAACACCCGAAUGGAGUCUCUAAGCUACAUAAUACCAACUUACACUUUACA |
| | | AAAUGUUGUCCCCCAAAAUGUAGCCAUUCGUAUCUGCUCCUAAUAAAAAGAAAGUUUCUUCACAUUCUCUCGAGAAAAAAAAA |
| | | AAAUGGAAAAAAAAAAAACGGAAAAAAAAAAAAGGUAAAAAAAAAAAAUAUAAAAAAAAAAAACAUAAAAAAAAAAAACGAAA |
| | | AAAAAAAAACGUAAAAAAAAAAAACUCAAAAAAAAAAAGAUAAAAAAAAAAAACCUAAAAAAAAAAAAUGUAAAAAAAAAAAA |
| | | GGGAAAAAAAAAAAACGCAAAAAAAAAAAACACAAAAAAAAAAAAUGCAAAAAAAAAAAAUCGAAAAAAAAAAAAUCUAAAAA |
| | | AAAAAAACGAAAAAAAAAAAACCCAAAAAAAAAAAAGACAAAAAAAAAAAAUAGAAAAAAAAAAAGUUAAAAAAAAAAAACUG |
| | | AAAAAAAAAAAAUUUAAAAAAAAAAAAUCUAG |
| mRNA O | 25 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGAGGACAAGCGGCCCGCCGCCACCAAGAAGGCCGGCCAGGCCAAGAAGAAGAAGGGCGGCUCCGGCGGCGGCGCCGC |
| Nme2Cas9 | | CUUCAAGCCCAACCCCAUCAACUACAUCCUGGGCCUGGACAUCGGCAUCGCCUCCGUGGGCUGGGCCAUGGUGGAGAUCGACG |
| | | AGGAGGAGAACCCCAUCCGGCUGAUCGACCUGGGCGUGCGGGUGUUCGAGCGGGCCGAGGUGCCCAAGACCGGCGACUCCCUG |
| | | GCCAUGGCCCGGCGGCUGGCCCGGUCCGUGCGGCGGCUGACCCGGCGGCGGGCCCACCGGCUGCUGCGGGCCCGGCGGCUGCU |
| | | GAAGCGGGAGGGCGUGCUGCAGGCCGCCGACUUCGACGAGAACGGCCUGAUCAAGUCCCUGCCCAACACCCCCUGGCAGCUGC |
| | | GGGCCGCCGCCCUGGACCGGAAGCUGACCCCCCUGGAGUGGUCCGCCGUGCUGCUGCACCUGAUCAAGCACCGGGGCUACCUG |
| | | UCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGG |
| | | GCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGAC |
| | | GAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGG |
| | | AGGGCCUGAAGGACAAGAAGUCCCCCCUGAACCUGUCCUCCGAGCUGCAGGACGAGAUCGGCACCGCCUUCUCCCUGUUCAAG |
| | | ACCGACGAGGACAUCACCGGCCGGCUGAAGGACCGGGUGCAGCCCGAGAUCCUGGAGGCCCUGCUGAAGCACAUCUCCUUCGA |
| | | CAAGUUCGUGCAGAUCUCCCUGAAGGCCCUGCGGCGGAUCGUGCCCCUGAUGGAGCAGGGCAAGCGGUACGACGAGGCCUGCG |
| | | CCGAGAUCUACGGCGACCACUACGGCAAGAAGAACACCGAGGAGAAGAUCUACCUGCCCCCCAUCCCCGCCGACGAGAUCCGG |
| | | AACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGG |
| | | ACCGGGAGAAGGCCGCCGCCAAGUUCCGGGAGUACUUCCCCAACUUCGUGGGCGAGCCCAAGUCCAAGGACAUCCUGAAGCUG |
| | | CGGCUGUACGAGCAGCAGCACGGCAAGUGCCUGUACUCCGGCAAGGAGAUCAACCUGGUGCGGCUGAACGAGAAGGGCUACGU |
| | | GGAGAUCGACCACGCCCUGCCCUUCUCCCGGACCUGGGACGACUCCUUCAACAACAAGGUGCUGGUGCUGGGCUCCGAGAACC |
| | | AGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGUGCAACCU |
| | | GAACGACACCCGGUACGUGAACCGGUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAU |
| | | GAACGCCUUCGACGGCAAGACCAUCGACAAGGAGACCGGCAAGGUGCUGCACCAGAAGACCCACUUCCCCCAGCCCUGGGAGU |
| | | UCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCG |
| | | UGAAGCGGGUGUGGCUGACCGAGAUCAAGCUGGCCGACCUGGAGAACAUGGUGAACUACAAGAACGGCCGGGAGAUCGAGCUG |
| | | UACGAGGCCCUGAAGGCCCGGCUGGAGGCCUACGGCGGCAACGCCAAGCAGGCCUUCGACCCCAAGGACAACCCCUUCUACAA |
| | | GAAGGGCGGCCAGCUGGUGAAGGCCGUGCGGGUGGAGAAGACCCAGGAGUCCGGCGUGCUGCUGAACAAGAAGAACGCCUACA |
| | | CCAUCGCCGACAACGGCGACAUGGUGCGGGUGGACGUGUUCUGCAAGGUGGACAAGAAGGGCAAGAACCAGUACUUCAUCGUG |
| | | CCCAUCUACGCCUGGCAGGUGGCCGAGAACAUCCUGCCCGACAUCGACUGCAAGGGCUACCGGAUCGACGACUCCUACACCUU |
| | | CUGCUUCUCCCUGCACAAGUACGACCUGAUCGCCUUCCAGAAGGACGAGAAGUCCAAGGUGGAGUUCGCCUACUACAUCAACU |
| | | GCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCG |
| | | GUAGCUAGCACCAGCCUCAAGAACACCCGAAUGGAGUCUCUAAGCUACAUAAUACCAACUUACACUUUACAAAAUGUUGUCCC |
| | | CCAAAAUGUAGCCAUUCGUAUCUGCUCCUAAUAAAAAGAAAGUUUCUUCACAUUCUCUCGAGAAAAAAAAAAAAUGGAAAAAA |
| | | AAAAAACGGAAAAAAAAAAAAGGUAAAAAAAAAAAAUAUAAAAAAAAAAAACAUAAAAAAAAAAAACGAAAAAAAAAAAACGU |
| | | AAAAAAAAAAAACUCAAAAAAAAAAAGAUAAAAAAAAAAAACCUAAAAAAAAAAAAUGUAAAAAAAAAAAAGGGAAAAAAAAA |
| | | AAACGCAAAAAAAAAAAACACAAAAAAAAAAAAUGCAAAAAAAAAAAAUCGAAAAAAAAAAAAUCUAAAAAAAAAAAACGAAA |
| | | AAAAAAAAACCCAAAAAAAAAAAAGACAAAAAAAAAAAAUAGAAAAAAAAAAAGUUAAAAAAAAAAAACUGAAAAAAAAAAAA |
| | | UUUAAAAAAAAAAAAUCUAG |
| mRNA P | 26 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGAGGACAAGCGGCCCGCCGCCACCAAGAAGGCCGGCCAGGCCAAGAAGAAGAAGGGCGGCUCCGGCGGCGGCGCCGC |
| Nme2Cas9 | | CUUCAAGCCCAACCCCAUCAACUACAUCCUGGGCCUGGACAUCGGCAUCGCCUCCGUGGGCUGGGCCAUGGUGGAGAUCGACG |
| with | | AGGAGGAGAACCCCAUCCGGCUGAUCGACCUGGGCGUGCGGGUGUUCGAGCGGGCCGAGGUGCCCAAGACCGGCGACUCCCUG |
| HiBiT tag | | GCCAUGGCCCGGCGGCUGGCCCGGUCCGUGCGGCGGCUGACCCGGCGGCGGGCCCACCGGCUGCUGCGGGCCCGGCGGCUGCU |
| | | GAAGCGGGAGGGCGUGCUGCAGGCCGCCGACUUCGACGAGAACGGCCUGAUCAAGUCCCUGCCCAACACCCCCUGGCAGCUGC |
| | | GGGCCGCCGCCCUGGACCGGAAGCUGACCCCCCUGGAGUGGUCCGCCGUGCUGCUGCACCUGAUCAAGCACCGGGGCUACCUG |
| | | UCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGG |
| | | GCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGAC |
| | | GAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGG |
| | | AGGGCCUGAAGGACAAGAAGUCCCCCCUGAACCUGUCCUCCGAGCUGCAGGACGAGAUCGGCACCGCCUUCUCCCUGUUCAAG |
| | | ACCGACGAGGACAUCACCGGCCGGCUGAAGGACCGGGUGCAGCCCGAGAUCCUGGAGGCCCUGCUGAAGCACAUCUCCUUCGA |
| | | CAAGUUCGUGCAGAUCUCCCUGAAGGCCCUGCGGCGGAUCGUGCCCCUGAUGGAGCAGGGCAAGCGGUACGACGAGGCCUGCG |
| | | CCGAGAUCUACGGCGACCACUACGGCAAGAAGAACACCGAGGAGAAGAUCUACCUGCCCCCCAUCCCCGCCGACGAGAUCCGG |
| | | AACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGG |
| | | ACCGGGAGAAGGCCGCCGCCAAGUUCCGGGAGUACUUCCCCAACUUCGUGGGCGAGCCCAAGUCCAAGGACAUCCUGAAGCUG |
| | | CGGCUGUACGAGCAGCAGCACGGCAAGUGCCUGUACUCCGGCAAGGAGAUCAACCUGGUGCGGCUGAACGAGAAGGGCUACGU |
| | | GGAGAUCGACCACGCCCUGCCCUUCUCCCGGACCUGGGACGACUCCUUCAACAACAAGGUGCUGGUGCUGGGCUCCGAGAACC |
| | | AGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGUGCAACCU |
| | | GAACGACACCCGGUACGUGAACCGGUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAU |
| | | GAACGCCUUCGACGGCAAGACCAUCGACAAGGAGACCGGCAAGGUGCUGCACCAGAAGACCCACUUCCCCCAGCCCUGGGAGU |
| | | UCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCG |
| | | UGAAGCGGGUGUGGCUGACCGAGAUCAAGCUGGCCGACCUGGAGAACAUGGUGAACUACAAGAACGGCCGGGAGAUCGAGCUG |
| | | UACGAGGCCCUGAAGGCCCGGCUGGAGGCCUACGGCGGCAACGCCAAGCAGGCCUUCGACCCCAAGGACAACCCCUUCUACAA |
| | | GAAGGGCGGCCAGCUGGUGAAGGCCGUGCGGGUGGAGAAGACCCAGGAGUCCGGCGUGCUGCUGAACAAGAAGAACGCCUACA |
| | | CCAUCGCCGACAACGGCGACAUGGUGCGGGUGGACGUGUUCUGCAAGGUGGACAAGAAGGGCAAGAACCAGUACUUCAUCGUG |
| | | CCCAUCUACGCCUGGCAGGUGGCCGAGAACAUCCUGCCCGACAUCGACUGCAAGGGCUACCGGAUCGACGACUCCUACACCUU |
| | | CUGCUUCUCCCUGCACAAGUACGACCUGAUCGCCUUCCAGAAGGACGAGAAGUCCAAGGUGGAGUUCGCCUACUACAUCAACU |
| | | GCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCG |
| | | GUCCGAGUCCGCCACCCCCGAGUCCGUGUCCGGCUGGCGGCUGUUCAAGAAGAUCUCCUAGCUAGCACCAGCCUCAAGAACAC |
| | | CCGAAUGGAGUCUCUAAGCUACAUAAUACCAACUUACACUUUACAAAAUGUUGUCCCCCAAAAUGUAGCCAUUCGUAUCUGCU |
| | | CCUAAUAAAAAGAAAGUUUCUUCACAUUCUCUCGAGAAAAAAAAAAAAUGGAAAAAAAAAAAACGGAAAAAAAAAAAAGGUAA |
| | | AAAAAAAAAAUAUAAAAAAAAAAAACAUAAAAAAAAAAAACGAAAAAAAAAAAACGUAAAAAAAAAAAACUCAAAAAAAAAAA |
| | | AGAUAAAAAAAAAAAACCUAAAAAAAAAAAAUGUAAAAAAAAAAAAGGGAAAAAAAAAAAACGCAAAAAAAAAAAACACAAAA |
| | | AAAAAAAAUGCAAAAAAAAAAAAUCGAAAAAAAAAAAAUCUAAAAAAAAAAAACGAAAAAAAAAAAACCCAAAAAAAAAAAAG |
| | | ACAAAAAAAAAAAAUAGAAAAAAAAAAAAGUUAAAAAAAAAAAACUGAAAAAAAAAAAAUUUAAAAAAAAAAAAUCUAG |
| mRNA Q | 27 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGGCGGCUCCGGCGGCGGCGCCGCCUUCAAGCCCAACCCCAUCAACUACAUCCUGGGCCUGGACAUCGGCAUCGCCUC |
| Nme2Cas9 | | CGUGGGCUGGGCCAUGGUGGAGAUCGACGAGGAGGAGAACCCCAUCCGGCUGAUCGACCUGGGCGUGCGGGUGUUCGAGCGGG |
| | | CCGAGGUGCCCAAGACCGGCGACUCCCUGGCCAUGGCCCGGCGGCUGGCCCGGUCCGUGCGGCGGCUGACCCGGCGGCGGGCC |
| | | CACCGGCUGCUGCGGGCCCGGCGGCUGCUGAAGCGGGAGGGCGUGCUGCAGGCCGCCGACUUCGACGAGAACGGCCUGAUCAA |
| | | GUCCCUGCCCAACACCCCCUGGCAGCUGCGGGCCGCCGCCCUGGACCGGAAGCUGACCCCCCUGGAGUGGUCCGCCGUGCUGC |
| | | UGCACCUGAUCAAGCACCGGGGCUACCUGUCCCAGCGGAAGAACGAGGGCGAGACCGCCGACAAGGAGCUGGGCGCCCUGCUG |
| | | AAGGGCGUGGCCAACAACGCCCACGCCCUGCAGACCGGCGACUUCCGGACCCCCGCCGAGCUGGCCCUGAACAAGUUCGAGAA |
| | | GGAGUCCGGCCACAUCCGGAACCAGCGGGGCGACUACUCCCACACCUUCUCCCGGAAGGACCUGCAGGCCGAGCUGAUCCUGC |
| | | UGUUCGAGAAGCAGAAGGAGUUCGGCAACCCCCACGUGUCCGGCGGCCUGAAGGAGGGCAUCGAGACCCUGCUGAUGACCCAG |
| | | CGGCCCGCCCUGUCCGGCGACGCCGUGCAGAAGAUGCUGGGCCACUGCACCUUCGAGCCCGCCGAGCCCAAGGCCGCCAAGAA |
| | | CACCUACACCGCCGAGCGGUUCAUCUGGCUGACCAAGCUGAACAACCUGCGGAUCCUGGAGCAGGGCUCCGAGCGGCCCCUGA |
| | | CCGACACCGAGCGGGCCACCCUGAUGGACGAGCCCUACCGGAAGUCCAAGCUGACCUACGCCCAGGCCCGGAAGCUGCUGGGC |
| | | CUGGAGGACACCGCCUUCUUCAAGGGCCUGCGGUACGGCAAGGACAACGCCGAGGCCUCCACCCUGAUGGAGAUGAAGGCCUA |
| | | CCACGCCAUCUCCCGGGCCCUGGAGAAGGAGGGCCUGAAGGACAAGAAGUCCCCCCUGAACCUGUCCUCCGAGCUGCAGGACG |
| | | AGAUCGGCACCGCCUUCUCCCUGUUCAAGACCGACGAGGACAUCACCGGCCGGCUGAAGGACCGGGUGCAGCCCGAGAUCCUG |
| | | GAGGCCCUGCUGAAGCACAUCUCCUUCGACAAGUUCGUGCAGAUCUCCCUGAAGGCCCUGCGGCGGAUCGUGCCCCUGAUGGA |
| | | GCAGGGCAAGCGGUACGACGAGGCCUGCGCCGAGAUCUACGGCGACCACUACGGCAAGAAGAACACCGAGGAGAAGAUCUACC |
| | | UGCCCCCCAUCCCCGCCGACGAGAUCCGGAACCCCGUGGUGCUGCGGGCCCUGUCCCAGGCCCGGAAGGUGAUCAACGGCGUG |
| | | GUGCGGCGGUACGGCUCCCCCGCCCGGAUCCACAUCGAGACCGCCCGGGAGGUGGGCAAGUCCUUCAAGGACCGGAAGGAGAU |
| | | CGAGAAGCGGCAGGAGGAGAACCGGAAGGACCGGGAGAAGGCCGCCGCCAAGUUCCGGGAGUACUUCCCCAACUUCGUGGGCG |
| | | AGCCCAAGUCCAAGGACAUCCUGAAGCUGCGGCUGUACGAGCAGCAGCACGGCAAGUGCCUGUACUCCGGCAAGGAGAUCAAC |
| | | CUGGUGCGGCUGAACGAGAAGGGCUACGUGGAGAUCGACCACGCCCUGCCCUUCUCCCGGACCUGGGACGACUCCUUCAACAA |
| | | CAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUC |
| | | GACGAGGACGGCUUCAAGGAGUGCAACCUGAACGACACCCGGUACGUGAACCGGUUCCUGUGCCAGUUCGUGGCCGACCACAU |
| | | CCUGCUGACCGGCAAGGGCAAGCGGCGGGUGUUCGCCUCCAACGGCCAGAUCACCAACCUGCUGCGGGGCUUCUGGGGCCUGC |
| | | GGAAGGUGCGGGCCGAGAACGACCGGCACCACGCCCUGGACGCCGUGGUGGUGGCCUGCUCCACCGUGGCCAUGCAGCAGAAG |
| | | AUCACCCGGUUCGUGCGGUACAAGGAGAUGAACGCCUUCGACGGCAAGACCAUCGACAAGGAGACCGGCAAGGUGCUGCACCA |
| | | GAAGACCCACUUCCCCCAGCCCUGGGAGUUCUUCGCCCAGGAGGUGAUGAUCCGGGUGUUCGGCAAGCCCGACGGCAAGCCCG |
| | | AGUUCGAGGAGGCCGACACCCCCGAGAAGCUGCGGACCCUGCUGGCCGAGAAGCUGUCCUCCCGGCCCGAGGCCGUGCACGAG |
| | | UACGUGACCCCCCUGUUCGUGUCCCGGGCCCCCAACCGGAAGAUGUCCGGCGCCCACAAGGACACCCUGCGGUCCGCCAAGCG |
| | | GUUCGUGAAGCACAACGAGAAGAUCUCCGUGAAGCGGGUGUGGCUGACCGAGAUCAAGCUGGCCGACCUGGAGAACAUGGUGA |
| | | ACUACAAGAACGGCCGGGAGAUCGAGCUGUACGAGGCCCUGAAGGCCCGGCUGGAGGCCUACGGCGGCAACGCCAAGCAGGCC |
| | | UUCGACCCCAAGGACAACCCCUUCUACAAGAAGGGCGGCCAGCUGGUGAAGGCCGUGCGGGUGGAGAAGACCCAGGAGUCCGG |
| | | CGUGCUGCUGAACAAGAAGAACGCCUACACCAUCGCCGACAACGGCGACAUGGUGCGGGUGGACGUGUUCUGCAAGGUGGACA |
| | | AGAAGGGCAAGAACCAGUACUUCAUCGUGCCCAUCUACGCCUGGCAGGUGGCCGAGAACAUCCUGCCCGACAUCGACUGCAAG |
| | | GGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGG |
| | | AGCAGCAGUUCCGGAUCUCCACCCAGAACCUGGUGCUGAUCCAGAAGUACCAGGUGAACGAGCUGGGCAAGGAGAUCCGGCCC |
| | | UGCCGGCUGAAGAAGCGGCCCCCCGUGCGGUAGCUAGCACCAGCCUCAAGAACACCCGAAUGGAGUCUCUAAGCUACAUAAUA |
| | | CCAACUUACACUUUACAAAAUGUUGUCCCCCAAAAUGUAGCCAUUCGUAUCUGCUCCUAAUAAAAAGAAAGUUUCUUCACAUU |
| | | CUCUCGAGAAAAAAAAAAAAUGGAAAAAAAAAAAACGGAAAAAAAAAAAAGGUAAAAAAAAAAAAUAUAAAAAAAAAAAACAU |
| | | AAAAAAAAAAAACGAAAAAAAAAAAACGUAAAAAAAAAAAACUCAAAAAAAAAAAGAUAAAAAAAAAAAACCUAAAAAAAAAA |
| | | AAUGUAAAAAAAAAAAAGGGAAAAAAAAAAAACGCAAAAAAAAAAAACACAAAAAAAAAAAAUGCAAAAAAAAAAAAUCGAAA |
| | | AAAAAAAAAUCUAAAAAAAAAAAACGAAAAAAAAAAAACCCAAAAAAAAAAAAGACAAAAAAAAAAAAUAGAAAAAAAAAAAG |
| | | UUAAAAAAAAAAAACUGAAAAAAAAAAAAUUUAAAAAAAAAAAAUCUAG |
| mRNA S | 28 | GGGAAGCUCAGAAUAAACGCUCAACUUUGGCCGGAUCUGCCACCAUGGACGGCUCCGGCGGCGGCUCCCCCAAGAAGAAGCGG |
| encoding | | AAGGUGGAGGACAAGCGGCCCGCCGCCACCAAGAAGGCCGGCCAGGCCAAGAAGAAGAAGGGCGGCUCCGGCGGCGGCGAGGC |
| Nme2Cas9 | | CUCCCCCGCCUCCGGCCCCCGGCACCUGAUGGACCCCCACAUCUUCACCUCCAACUUCAACAACGGCAUCGGCCGGCACAAGA |
| base | | CCUACCUGUGCUACGAGGUGGAGCGGCUGGACAACGGCACCUCCGUGAAGAUGGACCAGCACCGGGGCUUCCUGCACAACCAG |
| editor | | GCCAAGAACCUGCUGUGCGGCUUCUACGGCCGGCACGCCGAGCUGCGGUUCCUGGACCUGGUGCCCUCCCUGCAGCUGGACCC |
| | | CGCCCAGAUCUACCGGGUGACCUGGUUCAUCUCCUGGUCCCCCUGCUUCUCCUGGGGCUGCGCCGGCGAGGUGCGGGCCUUCC |
| | | UGCAGGAGAACACCCACGUGCGGCUGCGGAUCUUCGCCGCCCGGAUCUACGACUACGACCCCCUGUACAAGGAGGCCCUGCAG |
| | | AUGCUGCGGGACGCCGGCGCCCAGGUGUCCAUCAUGACCUACGACGAGUUCAAGCACUGCUGGGACACCUUCGUGGACCACCA |
| | | GGGCUGCCCCUUCCAGCCCUGGGACGGCCUGGACGAGCACUCCCAGGCCCUGUCCGGCCGGCUGCGGGCCAUCCUGCAGAACC |
| | | AGGGCAACUCCGGCUCCGAGACCCCCGGCACCUCCGAGUCCGCCACCCCCGAGUCCGCAGCGUUCAAACCAAAUCCCAUCAAC |
| | | UACAUCCUGGGCCUGGCCAUCGGCAUCGCCUCCGUGGGCUGGGCCAUGGUGGAGAUCGACGAGGAGGAGAACCCCAUCCGGCU |
| | | GAUCGACCUGGGCGUGCGGGUGUUCGAGCGGGCCGAGGUGCCCAAGACCGGCGACUCCCUGGCCAUGGCCCGGCGGCUGGCCC |
| | | GGUCCGUGCGGCGGCUGACCCGGCGGCGGGCCCACCGGCUGCUGCGGGCCCGGCGGCUGCUGAAGCGGGAGGGCGUGCUGCAG |
| | | GCCGCCGACUUCGACGAGAACGGCCUGAUCAAGUCCCUGCCCAACACCCCCUGGCAGCUGCGGGCCGCCGCCCUGGACCGGAA |
| | | GCUGACCCCCCUGGAGUGGUCCGCCGUGCUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCACGCCCUGCAGACCGGCGACUUCCGGACC |
| | | CCCGCCGAGCUGGCCCUGAACAAGUUCGAGAAGGAGUCCGGCCACAUCCGGAACCAGCGGGGCGACUACUCCCACACCUUCUC |
| | | CCGGAAGGACCUGCAGGCCGAGCUGAUCCUGCUGUUCGAGAAGCAGAAGGAGUUCGGCAACCCCCACGUGUCCGGCGGCCUGA |
| | | AGGAGGGCAUCGAGACCCUGCUGAUGACCCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCAUCUGGCUGACCAAGCUGAACAACCUGCG |
| | | GAUCCUGGAGCAGGGCUCCGAGCGGCCCCUGACCGACACCGAGCGGGCCACCCUGAUGGACGAGCCCUACCGGAAGUCCAAGC |
| | | UGACCUACGCCCAGGCCCGGAAGCUGCUGGGCCUGGAGGACACCGCCUUCUUCAAGGGCCUGCGGUACGGCAAGGACAACGCC |
| | | GAGGCCUCCACCCUGAUGGAGAUGAAGGCCUACCACGCCAUCUCCCGGGCCCUGGAGAAGGAGGGCCUGAAGGACAAGAAGUC |
| | | CCCCCUGAACCUGUCCUCCGAGCUGCAGGACGAGAUCGGCACCGCCUUCUCCCUGUUCAAGACCGACGAGGACAUCACCGGCC |
| | | GGCUGAAGGACCGGGUGCAGCCCGAGAUCCUGGAGGCCCUGCUGAAGCACAUCUCCUUCGACAAGUUCGUGCAGAUCUCCCUG |
| | | AAGGCCCUGCGGCGGAUCGUGCCCCUGAUGGAGCAGGGCAAGCGGUACGACGAGGCCUGCGCCGAGAUCUACGGCGACCACUA |
| | | CGGCAAGAAGAACACCGAGGAGAAGAUCUACCUGCCCCCCAUCCCCGCCGACGAGAUCCGGAACCCCGUGGUGCUGCGGGCCC |
| | | UGUCCCAGGCCCGGAAGGUGAUCAACGGCGUGGUGCGGCGGUACGGCUCCCCCGCCCGGAUCCACAUCGAGACCGCCCGGGAG |
| | | GUGGGCAAGUCCUUCAAGGACCGGAAGGAGAUCGAGAAGCGGCAGGAGGAGAACCGGAAGGACCGGGAGAAGGCCGCCGCCAA |
| | | GUUCCGGGAGUACUUCCCCAACUUCGUGGGCGAGCCCAAGUCCAAGGACAUCCUGAAGCUGCGGCUGUACGAGCAGCAGCACG |
| | | GCAAGUGCCUGUACUCCGGCAAGGAGAUCAACCUGGUGCGGCUGAACGAGAAGGGCUACGUGGAGAUCGACCACGCCCUGCCC |
| | | UUCUCCCGGACCUGGGACGACUCCUUCAACAACAAGGUGCUGGUGCUGGGCUCCGAGAACCAGAACAAGGGCAACCAGACCCC |
| | | CUACGAGUACUUCAACGGCAAGGACAACUCCCGGGAGUGGCAGGAGUUCAAGGCCCGGGUGGAGACCUCCCGGUUCCCCCGGU |
| | | CCAAGAAGCAGCGGAUCCUGCUGCAGAAGUUCGACGAGGACGGCUUCAAGGAGUGCAACCUGAACGACACCCGGUACGUGAAC |
| | | CGCUUCCUGUGCCAGUUCGUGGCCGACCACAUCCUGCUGACCGGCAAGGGCAAGCGGCGGGUGUUCGCCUCCAACGGCCAGAU |
| | | CACCAACCUGCUGCGGGGCUUCUGGGGCCUGCGGAAGGUGCGGGCCGAGAACGACCGGCACCACGCCCUGGACGCCGUGGUGG |
| | | UGGCCUGCUCCACCGUGGCCAUGCAGCAGAAGAUCACCCGGUUCGUGCGGUACAAGGAGAUGAACGCCUUCGACGGCAAGACC |
| | | AUCGACAAGGAGACCGGCAAGGUGCUGCACCAGAAGACCCACUUCCCCCAGCCCUGGGAGUUCUUCGCCCAGGAGGUGAUGAU |
| | | CCGGGUGUUCGGCAAGCCCGACGGCAAGCCCGAGUUCGAGGAGGCCGACACCCCCGAGAAGCUGCGGACCCUGCUGGCCGAGA |
| | | AGCUGUCCUCCCGGCCCGAGGCCGUGCACGAGUACGUGACCCCCCUGUUCGUGUCCCGGGCCCCCAACCGGAAGAUGUCCGGC |
| | | GCCCACAAGGACACCCUGCGGUCCGCCAAGCGGUUCGUGAAGCACAACGAGAAGAUCUCCGUGAAGCGGGUGUGGCUGACCGA |
| | | GAUCAAGCUGGCCGACCUGGAGAACAUGGUGAACUACAAGAACGGCCGGGAGAUCGAGCUGUACGAGGCCCUGAAGGCCCGGC |
| | | UGGAGGCCUACGGCGGCAACGCCAAGCAGGCCUUCGACCCCAAGGACAACCCCUUCUACAAGAAGGGCGGCCAGCUGGUGAAG |
| | | GCCGUGCGGGUGGAGAAGACCCAGGAGUCCGGCGUGCUGCUGAACAAGAAGAACGCCUACACCAUCGCCGACAACGGCGACAU |
| | | GGUGCGGGUGGACGUGUUCUGCAAGGUGGACAAGAAGGGCAAGAACCAGUACUUCAUCGUGCCCAUCUACGCCUGGCAGGUGG |
| | | CCGAGAACAUCCUGCCCGACAUCGACUGCAAGGGCUACCGGAUCGACGACUCCUACACCUUCUGCUUCUCCCUGCACAAGUAC |
| | | GACCUGAUCGCCUUCCAGAAGGACGAGAAGUCCAAGGUGGAGUUCGCCUACUACAUCAACUGCGACUCCUCCAACGGCCGGUU |
| | | CUACCUGGCCUGGCACGACAAGGGCUCCAAGGAGCAGCAGUUCCGGAUCUCCACCCAGAACCUGGUGCUGAUCCAGAAGUACC |
| | | AGGUGAACGAGCUGGGCAAGGAGAUCCGGCCCUGCCGGCUGAAGAAGCGGCCCCCCGUGCGGUAGUGACUAGCACCAGCCUCA |
| | | AGAACACCCGAAUGGAGUCUCUAAGCUACAUAAUACCAACUUACACUUUACAAAAUGUUGUCCCCCAAAAUGUAGCCAUUCGU |
| | | AUCUGCUCCUAAUAAAAAGAAAGUUUCUUCACAUUCUCUCGAGAAAAAAAAAAAAUGGAAAAAAAAAAAACGGAAAAAAAAAA |
| | | AGGUAAAAAAAAAAAAUAUAAAAAAAAAAACAUAAAAAAAAAAAACGAAAAAAAAAAAACGUAAAAAAAAAAAACUCAAAAAA |
| | | AAAAAGAUAAAAAAAAAAAACCUAAAAAAAAAAAAUGUAAAAAAAAAAAAGGGAAAAAAAAAAACGCAAAAAAAAAAAACACA |
| | | AAAAAAAAAAAUGCAAAAAAAAAAAAUCGAAAAAAAAAAAAUCUAAAAAAAAAAAACGAAAAAAAAAAAACCCAAAAAAAAAA |
| | | AAGACAAAAAAAAAAAAUAGAAAAAAAAAAAGUUAAAAAAAAAAAACUGAAAAAAAAAAAAUUUAAAAAAAAAAAAUCUAG |
| Open | 29 | atgaccggtgccgccttcaagcccaaccccatcaactacatcctgggcctggacatcggcatcgcctccgtgggctgggccat |
| reading | | ggtggagatcgacgaggaggagaaccccatccggctgatcgacctgggcgtgcgggtgttcgagcgggccgaggtgcccaaga |
| frame for | | ccggcgactccctggccatggcccggcggctggcccggtccgtgcggcggctgacccggcggcgggcccaccggctgctgcgg |
| Nme2Cas9 | | gcccggcggctgctgaagcgggagggcgtgctgcaggccgccgacttcgacgagaacggcctgatcaagtccctgcccaacac |
| encoded | | cccctggcagctgcgggccgccgccctggaccggaagctgacccccctggagtggtccgccgtgctgctgcacctgatcaagc |
| by mRNA C | | accggggctacctgtcccagcggaagaacgagggcgagaccgccgacaaggagctgggcgccctgctgaagggcgtggccaac |
| | | aacgcccacgccctgcagaccggcgacttccggacccccgccgagctggccctgaacaagttcgagaaggagtccggccacat |
| | | ccggaaccagcggggcgactactcccacaccttctcccggaaggacctgcaggccgagctgatcctgctgttcgagaagcaga |
| | | aggagttcggcaacccccacgtgtccggcggcctgaaggagggcatcgagaccctgctgatgacccagcggcccgccctgtcc |
| | | ggcgacgccgtgcagaagatgctgggccactgcaccttcgagcccgccgagcccaaggccgccaagaacacctacaccgccga |
| | | gcggttcatctggctgaccaagctgaacaacctgcggatcctggagcagggctccgagcggcccctgaccgacaccgagcggg |
| | | ccaccctgatggacgagccctaccggaagtccaagctgacctacgcccaggcccggaagctgctgggcctggaggacaccgcc |
| | | ttcttcaagggcctgcggtacggcaaggacaacgccgaggcctccaccctgatggagatgaaggcctaccacgccatctcccg |
| | | ggccctggagaaggagggcctgaaggacaagaagtcccccctgaacctgtcctccgagctgcaggacgagatcggcaccgcct |
| | | tctccctgttcaagaccgacgaggacatcaccggccggctgaaggaccgggtgcagcccgagatcctggaggccctgctgaag |
| | | cacatctccttcgacaagttcgtgcagatctccctgaaggccctgcggcggatcgtgcccctgatggagcagggcaagcggta |
| | | cgacgaggcctgcgccgagatctacggcgaccactacggcaagaagaacaccgaggagaagatctacctgccccccatccccg |
| | | ccgacgagatccggaaccccgtggtgctgcgggccctgtcccaggcccggaaggtgatcaacggcgtggtgcggcggtacggc |
| | | tcccccgcccggatccacatcgagaccgcccgggaggtgggcaagtccttcaaggaccggaaggagatcgagaagcggcagga |
| | | ggagaaccggaaggaccgggagaaggccgccgccaagttccgggagtacttccccaacttcgtgggcgagcccaagtccaagg |
| | | acatcctgaagctgcggctgtacgagcagcagcacggcaagtgcctgtactccggcaaggagatcaacctggtgcggctgaac |
| | | gagaagggctacgtggagatcgaccacgccctgcccttctcccggacctgggacgactccttcaacaacaaggtgctggtgct |
| | | gggctccgagaaccagaacaagggcaaccagaccccctacgagtacttcaacggcaaggacaactcccgggagtggcaggagt |
| | | tcaaggcccgggtggagacctcccggttcccccggtccaagaagcagcggatcctgctgcagaagttcgacgaggacggcttc |
| | | aaggagtgcaacctgaacgacacccggtacgtgaaccgcttcctgtgccagttcgtggccgaccacatcctgctgaccggcaa |
| | | gggcaagcggcgggtgttcgcctccaacggccagatcaccaacctgctgcggggcttctggggcctgcggaaggtgcgggccg |
| | | agaacgaccggcaccacgccctggacgccgtggtggtggcctgctccaccgtggccatgcagcagaagatcacccggttcgtg |
| | | cggtacaaggagatgaacgccttcgacggcaagaccatcgacaaggagaccggcaaggtgctgcaccagaagacccacttccc |
| | | ccagccctgggagttcttcgcccaggaggtgatgatccgggtgttcggcaagcccgacggcaagcccgagttcgaggaggccg |
| | | acacccccgagaagctgcggaccctgctggccgagaagctgtcctcccggcccgaggccgtgcacgagtacgtgacccccctg |
| | | ttcgtgtcccgggcccccaaccggaagatgtccggcgcccacaaggacaccctgcggtccgccaagcggttcgtgaagcacaa |
| | | cgagaagatctccgtgaagcgggtgtggctgaccgagatcaagctggccgacctggagaacatggtgaactacaagaacggcc |
| | | gggagatcgagctgtacgaggccctgaaggcccggctggaggcctacggcggcaacgccaagcaggccttcgaccccaaggac |
| | | aaccccttctacaagaagggcggccagctggtgaaggccgtgcgggtggagaagacccaggagtccggcgtgctgctgaacaa |
| | | gaagaacgcctacaccatcgccgacaacggcgacatggtgcgggtggacgtgttctgcaaggtggacaagaagggcaagaacc |
| | | agtacttcatcgtgcccatctacgcctggcaggtggccgagaacatcctgcccgacatcgactgcaagggctaccggatcgac |
| | | gactcctacaccttctgcttctccctgcacaagtacgacctgatcgccttccagaaggacgagaagtccaaggtggagttcgc |
| | | ctactacatcaactgcgactcctccaacggccggttctacctggcctggcacgacaagggctccaaggagcagcagttccgga |
| | | tctccacccagaacctggtgctgatccagaagtaccaggtgaacgagctgggcaaggagatccggccctgccggctgaagaag |
| | | cggccccccgtgcggtccggaaagcggaccgccgacggctccgagttcgagtcccccaagaagaagcggaaggtggagtag |
| Open | 30 | ATGgaggcctcccccgcctccggcccccggcacctgatggacccccacatcttcacctccAACTTCAACAACggcATCggccg |
| reading | | gCACAAGaccTACCTGTGCTACgaggtggagcggCTGGACAACggcacctccgtgAAGATGGACCAGCACcggggcTTCCTGC |
| frame for | | ACAACCAGgccAAGAACCTGCTGTGCggcTTCTACggccggCACgccgagCTGcggTTCCTGGACCTGgtgccctccCTGCAG |
| SpyCas9 | | CTGGACcccgccCAGATCTACcgggtgaccTGGTTCATCtccTGGtcccccTGCTTCtccTGGggcTGCgccggcgaggtgcg |
| base | | ggccTTCCTGCAGgagAACaccCACgtgcggCTGcggATCTTCgccgcccggATCTACGACTACGACcccCTGTACAAGgagg |
| editor | | CCCTGCAGATGCTGcggGACgccggcgccCAGgtgtccATCATGaccTACGACgagTTCAAGCACTGCTGGGACaccTTCgtg |
| encoded | | GACCACCAGggcTGCcccTTCCAGcccTGGGACggcCTGGACgagCACtccCAGgccCTGtccggccggCTGcgggccATCCT |
| by mRNA E | | GCAGAACCAGggcAACtccggctccgagacccccggcacctccgagtccgccacccccgagtccgacaagaagtactccatcg |
| | | gcctggCcatcggcaccaactccgtgggctgggccgtgatcaccgacgagtacaaggtgccctccaagaagttcaaggtgctg |
| | | ggcaacaccgaccggcactccatcaagaagaacctgatcggcgccctgctgttcgactccggcgagaccgccgaggccacccg |
| | | gctgaagcggaccgcccggcggcggtacacccggcggaagaaccggatctgctacctgcaggagatcttctccaacgagatgg |
| | | ccaaggtggacgactccttcttccaccggctggaggagtccttcctggtggaggaggacaagaagcacgagcggcaccccatc |
| | | ttcggcaacatcgtggacgaggtggcctaccacgagaagtaccccaccatctaccacctgcggaagaagctggtggactccac |
| | | cgacaaggccgacctgcggctgatctacctggccctggcccacatgatcaagttccggggccacttcctgatcgagggcgacc |
| | | tgaaccccgacaactccgacgtggacaagctgttcatccagctggtgcagacctacaaccagctgttcgaggagaaccccatc |
| | | aacgcctccggcgtggacgccaaggccatcctgtccgcccggctgtccaagtcccggcggctggagaacctgatcgcccagct |
| | | gcccggcgagaagaagaacggcctgttcggcaacctgatcgccctgtccctgggcctgacccccaacttcaagtccaacttcg |
| | | acctggccgaggacgccaagctgcagctgtccaaggacacctacgacgacgacctggacaacctgctggcccagatcggcgac |
| | | cagtacgccgacctgttcctggccgccaagaacctgtccgacgccatcctgctgtccgacatcctgcgggtgaacaccgagat |
| | | caccaaggcccccctgtccgcctccatgatcaagcggtacgacgagcaccaccaggacctgaccctgctgaaggccctggtgc |
| | | ggcagcagctgcccgagaagtacaaggagatcttcttcgaccagtccaagaacggctacgccggctacatcgacggcggcgcc |
| | | tcccaggaggagttctacaagttcatcaagcccatcctggagaagatggacggcaccgaggagctgctggtgaagctgaaccg |
| | | ggaggacctgctgcggaagcagcggaccttcgacaacggctccatcccccaccagatccacctgggcgagctgcacgccatcc |
| | | tgcggcggcaggaggacttctaccccttcctgaaggacaaccgggagaagatcgagaagatcctgaccttccggatcccctac |
| | | tacgtgggccccctggcccggggcaactcccggttcgcctggatgacccggaagtccgaggagaccatcaccccctggaactt |
| | | cgaggaggtggtggacaagggcgcctccgcccagtccttcatcgagcggatgaccaacttcgacaagaacctgcccaacgaga |
| | | aggtgctgcccaagcactccctgctgtacgagtacttcaccgtgtacaacgagctgaccaaggtgaagtacgtgaccgagggc |
| | | atgcggaagcccgccttcctgtccggcgagcagaagaaggccatcgtggacctgctgttcaagaccaaccggaaggtgaccgt |
| | | gaagcagctgaaggaggactacttcaagaagatcgagtgcttcgactccgtggagatctccggcgtggaggaccggttcaacg |
| | | cctccctgggcacctaccacgacctgctgaagatcatcaaggacaaggacttcctggacaacgaggagaacgaggacatcctg |
| | | gaggacatcgtgctgaccctgaccctgttcgaggaccgggagatgatcgaggagcggctgaagacctacgcccacctgttcga |
| | | cgacaaggtgatgaagcagctgaagcggcggcggtacaccggctggggccggctgtcccggaagctgatcaacggcatccggg |
| | | acaagcagtccggcaagaccatcctggacttcctgaagtccgacggcttcgccaaccggaacttcatgcagctgatccacgac |
| | | gactccctgaccttcaaggaggacatccagaaggcccaggtgtccggccagggcgactccctgcacgagcacatcgccaacct |
| | | ggccggctcccccgccatcaagaagggcatcctgcagaccgtgaaggtggtggacgagctggtgaaggtgatgggccggcaca |
| | | agcccgagaacatcgtgatcgagatggcccgggagaaccagaccacccagaagggccagaagaactcccgggagcggatgaag |
| | | cggatcgaggagggcatcaaggagctgggctcccagatcctgaaggagcaccccgtggagaacacccagctgcagaacgagaa |
| | | gctgtacctgtactacctgcagaacggccgggacatgtacgtggaccaggagctggacatcaaccggctgtccgactacgacg |
| | | tggaccacatcgtgccccagtccttcctgaaggacgactccatcgacaacaaggtgctgacccggtccgacaagaaccggggc |
| | | aagtccgacaacgtgccctccgaggaggtggtgaagaagatgaagaactactggcggcagctgctgaacgccaagctgatcac |
| | | ccagcggaagttcgacaacctgaccaaggccgagcggggcggcctgtccgagctggacaaggccggcttcatcaagcggcagc |
| | | tggtggagacccggcagatcaccaagcacgtggcccagatcctggactcccggatgaacaccaagtacgacgagaacgacaag |
| | | ctgatccgggaggtgaaggtgatcaccctgaagtccaagctggtgtccgacttccggaaggacttccagttctacaaggtgcg |
| | | ggagatcaacaactaccaccacgcccacgacgcctacctgaacgccgtggtgggcaccgccctgatcaagaagtaccccaagc |
| | | tggagtccgagttcgtgtacggcgactacaaggtgtacgacgtgcggaagatgatcgccaagtccgagcaggagatcggcaag |
| | | gccaccgccaagtacttcttctactccaacatcatgaacttcttcaagaccgagatcaccctggccaacggcgagatccggaa |
| | | gcggcccctgatcgagaccaacggcgagaccggcgagatcgtgtgggacaagggccgggacttcgccaccgtgcggaaggtgc |
| | | tgtccatgccccaggtgaacatcgtgaagaagaccgaggtgcagaccggcggcttctccaaggagtccatcctgcccaagcgg |
| | | aactccgacaagctgatcgcccggaagaaggactgggaccccaagaagtacggcggcttcgactcccccaccgtggcctactc |
| | | cgtgctggtggtggccaaggtggagaagggcaagtccaagaagctgaagtccgtgaaggagctgctgggcatcaccatcatgg |
| | | agcggtcctccttcgagaagaaccccatcgacttcctggaggccaagggctacaaggaggtgaagaaggacctgatcatcaag |
| | | ctgcccaagtactccctgttcgagctggagaacggccggaagcggatgctggcctccgccggcgagctgcagaagggcaacga |
| | | gctggccctgccctccaagtacgtgaacttcctgtacctggcctcccactacgagaagctgaagggctcccccgaggacaacg |
| | | agcagaagcagctgttcgtggagcagcacaagcactacctggacgagatcatcgagcagatctccgagttctccaagcgggtg |
| | | atcctggccgacgccaacctggacaaggtgctgtccgcctacaacaagcaccgggacaagcccatccgggagcaggccgagaa |
| | | catcatccacctgttcaccctgaccaacctgggcgcccccgccgccttcaagtacttcgacaccaccatcgaccggaagcggt |
| | | acacctccaccaaggaggtgctggacgccaccctgatccaccagtccatcaccggcctgtacgagacccggatcgacctgtcc |
| | | cagctgggcggcgacggcggcggctcccccaagaagaagcggaaggtgTgA |
| Open | 31 | ATGACCAACCTGTCCGACATCATCGAGAAGGAGACCGGCAAGCAGCTGGTGATCCAGGAGTCCATCCTGATGCTGCCCGAGGA |
| reading | | GGTGGAGGAGGTGATCGGCAACAAGCCCGAGTCCGACATCCTGGTGCACACCGCCTACGACGAGTCCACCGACGAGAACGTGA |
| frame for | | TGCTGCTGACCTCCGACGCCCCCGAGTACAAGCCCTGGGCCCTGGTGATCCAGGACTCCAACGGCGAGAACAAGATCAAGATG |
| UGI | | CTGTCCGGCGGCTCCAAGCGGACCGCCGACGGCTCCGAGTTCGAGTCCCCCAAGAAGAAGCGGAAGGTGGAGTGATAG |
| encoded | | |
| by mRNA G | | |
| Open | 32 | ATGGCCGCCTTCAAGCCCAACCCCATCAACTACATCCTGGGCCTGGACATCGGCATCGCCTCCGTGGGCTGGGCCATGGTGGA |
| reading | | GATCGACGAGGAGGAGAACCCCATCCGGCTGATCGACCTGGGCGTGCGGGTGTTCGAGCGGGCCGAGGTGCCCAAGACCGGCG |
| frame for | | ACTCCCTGGCCATGGCCCGGCGGCTGGCCCGGTCCGTGCGGCGGCTGACCCGGCGGCGGGCCCACCGGCTGCTGCGGGCCCGG |
| Nme2Cas9 | | CGGCTGCTGAAGCGGGAGGGCGTGCTGCAGGCCGCCGACTTCGACGAGAACGGCCTGATCAAGTCCCTGCCCAACACCCCCTG |
| encoded | | GCAGCTGCGGGCCGCCGCCCTGGACCGGAAGCTGACCCCCCTGGAGTGGTCCGCCGTGCTGCTGCACCTGATCAAGCACCGGG |
| by mRNA H | | GCTACCTGTCCCAGCGGAAGAACGAGGGCGAGACCGCCGACAAGGAGCTGGGCGCCCTGCTGAAGGGCGTGGCCAACAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGTCCGGCGGCGGCTCCCCCA |
| | | AGAAGAAGCGGAAGGTGTCCGGCGGCTCCGGCAAGAACCAGTACTTCATCGTGCCCATCTACGCCTGGCAGGTGGCCGAGAAC |
| | | ATCCTGCCCGACATCGACTGCAAGGGCTACCGGATCGACGACTCCTACACCTTCTGCTTCTCCCTGCACAAGTACGACCTGAT |
| | | CGCCTTCCAGAAGGACGAGAAGTCCAAGGTGGAGTTCGCCTACTACATCAACTGCGACTCCTCCAACGGCCGGTTCTACCTGG |
| | | CCTGGCACGACAAGGGCTCCAAGGAGCAGCAGTTCCGGATCTCCACCCAGAACCTGGTGCTGATCCAGAAGTACCAGGTGAAC |
| | | GAGCTGGGCAAGGAGATCCGGCCCTGCCGGCTGAAGAAGCGGCCCCCCGTGCGGTAG |
| Open | 33 | ATGGTGCCCAAGAAGAAGCGGAAGGTGGCCGCCTTCAAGCCCAACCCCATCAACTACATCCTGGGCCTGGACATCGGCATCGC |
| reading | | CTCCGTGGGCTGGGCCATGGTGGAGATCGACGAGGAGGAGAACCCCATCCGGCTGATCGACCTGGGCGTGCGGGTGTTCGAGC |
| frame for | | GGGCCGAGGTGCCCAAGACCGGCGACTCCCTGGCCATGGCCCGGCGGCTGGCCCGGTCCGTGCGGCGGCTGACCCGGCGGCGG |
| Nme2Cas9 | | GCCCACCGGCTGCTGCGGGCCCGGCGGCTGCTGAAGCGGGAGGGCGTGCTGCAGGCCGCCGACTTCGACGAGAACGGCCTGAT |
| encoded | | CAAGTCCCTGCCCAACACCCCCTGGCAGCTGCGGGCCGCCGCCCTGGACCGGAAGCTGACCCCCCTGGAGTGGTCCGCCGTGC |
| by mRNA I | | TGCTGCACCTGATCAAGCACCGGGGCTACCTGTCCCAGCGGAAGAACGAGGGCGAGACCGCCGACAAGGAGCTGGGCGCCCTG |
| | | CTGAAGGGCGTGGCCAACAACGCCCACGCCCTGCAGACCGGCGACTTCCGGACCCCCGCCGAGCTGGCCCTGAACAAGTTCGA |
| | | GAAGGAGTCCGGCCACATCCGGAACCAGCGGGGCGACTACTCCCACACCTTCTCCCGGAAGGACCTGCAGGCCGAGCTGATCC |
| | | TGCTGTTCGAGAAGCAGAAGGAGTTCGGCAACCCCCACGTGTCCGGCGGCCTGAAGGAGGGCATCGAGACCCTGCTGATGACC |
| | | CAGCGGCCCGCCCTGTCCGGCGACGCCGTGCAGAAGATGCTGGGCCACTGCACCTTCGAGCCCGCCGAGCCCAAGGCCGCCAA |
| | | GAACACCTACACCGCCGAGCGGTTCATCTGGCTGACCAAGCTGAACAACCTGCGGATCCTGGAGCAGGGCTCCGAGCGGCCCC |
| | | TGACCGACACCGAGCGGGCCACCCTGATGGACGAGCCCTACCGGAAGTCCAAGCTGACCTACGCCCAGGCCCGGAAGCTGCTG |
| | | GGCCTGGAGGACACCGCCTTCTTCAAGGGCCTGCGGTACGGCAAGGACAACGCCGAGGCCTCCACCCTGATGGAGATGAAGGC |
| | | CTACCACGCCATCTCCCGGGCCCTGGAGAAGGAGGGCCTGAAGGACAAGAAGTCCCCCCTGAACCTGTCCTCCGAGCTGCAGG |
| | | ACGAGATCGGCACCGCCTTCTCCCTGTTCAAGACCGACGAGGACATCACCGGCCGGCTGAAGGACCGGGTGCAGCCCGAGATC |
| | | CTGGAGGCCCTGCTGAAGCACATCTCCTTCGACAAGTTCGTGCAGATCTCCCTGAAGGCCCTGCGGCGGATCGTGCCCCTGAT |
| | | GGAGCAGGGCAAGCGGTACGACGAGGCCTGCGCCGAGATCTACGGCGACCACTACGGCAAGAAGAACACCGAGGAGAAGATCT |
| | | ACCTGCCCCCCATCCCCGCCGACGAGATCCGGAACCCCGTGGTGCTGCGGGCCCTGTCCCAGGCCCGGAAGGTGATCAACGGC |
| | | GTGGTGCGGCGGTACGGCTCCCCCGCCCGGATCCACATCGAGACCGCCCGGGAGGTGGGCAAGTCCTTCAAGGACCGGAAGGA |
| | | GATCGAGAAGCGGCAGGAGGAGAACCGGAAGGACCGGGAGAAGGCCGCCGCCAAGTTCCGGGAGTACTTCCCCAACTTCGTGG |
| | | GCGAGCCCAAGTCCAAGGACATCCTGAAGCTGCGGCTGTACGAGCAGCAGCACGGCAAGTGCCTGTACTCCGGCAAGGAGATC |
| | | AACCTGGTGCGGCTGAACGAGAAGGGCTACGTGGAGATCGACCACGCCCTGCCCTTCTCCCGGACCTGGGACGACTCCTTCAA |
| | | CAACAAGGTGCTGGTGCTGGGCTCCGAGAACCAGAACAAGGGCAACCAGACCCCCTACGAGTACTTCAACGGCAAGGACAACT |
| | | CCCGGGAGTGGCAGGAGTTCAAGGCCCGGGTGGAGACCTCCCGGTTCCCCCGGTCCAAGAAGCAGCGGATCCTGCTGCAGAAG |
| | | TTCGACGAGGACGGCTTCAAGGAGTGCAACCTGAACGACACCCGGTACGTGAACCGGTTCCTGTGCCAGTTCGTGGCCGACCA |
| | | CATCCTGCTGACCGGCAAGGGCAAGCGGCGGGTGTTCGCCTCCAACGGCCAGATCACCAACCTGCTGCGGGGCTTCTGGGGCC |
| | | TGCGGAAGGTGCGGGCCGAGAACGACCGGCACCACGCCCTGGACGCCGTGGTGGTGGCCTGCTCCACCGTGGCCATGCAGCAG |
| | | AAGATCACCCGGTTCGTGCGGTACAAGGAGATGAACGCCTTCGACGGCAAGACCATCGACAAGGAGACCGGCAAGGTGCTGCA |
| | | CCAGAAGACCCACTTCCCCCAGCCCTGGGAGTTCTTCGCCCAGGAGGTGATGATCCGGGTGTTCGGCAAGCCCGACGGCAAGC |
| | | CCGAGTTCGAGGAGGCCGACACCCCCGAGAAGCTGCGGACCCTGCTGGCCGAGAAGCTGTCCTCCCGGCCCGAGGCCGTGCAC |
| | | GAGTACGTGACCCCCCTGTTCGTGTCCCGGGCCCCCAACCGGAAGATGTCCGGCGCCCACAAGGACACCCTGCGGTCCGCCAA |
| | | GCGGTTCGTGAAGCACAACGAGAAGATCTCCGTGAAGCGGGTGTGGCTGACCGAGATCAAGCTGGCCGACCTGGAGAACATGG |
| | | TGAACTACAAGAACGGCCGGGAGATCGAGCTGTACGAGGCCCTGAAGGCCCGGCTGGAGGCCTACGGCGGCAACGCCAAGCAG |
| | | GCCTTCGACCCCAAGGACAACCCCTTCTACAAGAAGGGCGGCCAGCTGGTGAAGGCCGTGCGGGTGGAGAAGACCCAGGAGTC |
| | | CGGCGTGCTGCTGAACAAGAAGAACGCCTACACCATCGCCGACAACGGCGACATGGTGCGGGTGGACGTGTTCTGCAAGGTGG |
| | | ACAAGAAGGGCAAGAACCAGTACTTCATCGTGCCCATCTACGCCTGGCAGGTGGCCGAGAACATCCTGCCCGACATCGACTGC |
| | | AAGGGCTACCGGATCGACGACTCCTACACCTTCTGCTTCTCCCTGCACAAGTACGACCTGATCGCCTTCCAGAAGGACGAGAA |
| | | GTCCAAGGTGGAGTTCGCCTACTACATCAACTGCGACTCCTCCAACGGCCGGTTCTACCTGGCCTGGCACGACAAGGGCTCCA |
| | | AGGAGCAGCAGTTCCGGATCTCCACCCAGAACCTGGTGCTGATCCAGAAGTACCAGGTGAACGAGCTGGGCAAGGAGATCCGG |
| | | CCCTGCCGGCTGAAGAAGCGGCCCCCCGTGCGGTACCCCTACGACGTGCCCGACTACGCCGCCGCCCCCGCCGCCAAGAAGAA |
| | | GAAGCTGGACTAG |
| Open | 34 | ATGGCCGCCTTCAAGCCCAACCCCATCAACTACATCCTGGGCCTGGACATCGGCATCGCCTCCGTGGGCTGGGCCATGGTGGA |
| reading | | GATCGACGAGGAGGAGAACCCCATCCGGCTGATCGACCTGGGCGTGCGGGTGTTCGAGCGGGCCGAGGTGCCCAAGACCGGCG |
| frame for | | ACTCCCTGGCCATGGCCCGGCGGCTGGCCCGGTCCGTGCGGCGGCTGACCCGGCGGCGGGCCCACCGGCTGCTGCGGGCCCGG |
| Nme2Cas9 | | CGGCTGCTGAAGCGGGAGGGCGTGCTGCAGGCCGCCGACTTCGACGAGAACGGCCTGATCAAGTCCCTGCCCAACACCCCCTG |
| encoded | | GCAGCTGCGGGCCGCCGCCCTGGACCGGAAGCTGACCCCCCTGGAGTGGTCCGCCGTGCTGCTGCACCTGATCAAGCACCGGG |
| by mRNA J | | GCTACCTGTCCCAGCGGAAGAACGAGGGCGAGACCGCCGACAAGGAGCTGGGCGCCCTGCTGAAGGGCGTGGCCAACAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGG |
| Open | 35 | atggccgccttcaagcccaaccccatcaactacatcctgggcctggacatcggcatcgcctccgtgggctgggccatggtgga |
| reading | | gatcgacgaggaggagaaccccatccggctgatcgacctgggcgtgcgggtgttcgagcgggccgaggtgcccaagaccggcg |
| frame for | | actccctggccatggcccggcggctggcccggtccgtgcggcggctgacccggcggcgggcccaccggctgctgcgggcccgg |
| Nme2Cas9 | | cggctgctgaagcgggagggcgtgctgcaggccgccgacttcgacgagaacggcctgatcaagtccctgcccaacaccccctg |
| encoded | | gcagctgcgggccgccgccctggaccggaagctgacccccctggagtggtccgccgtgctgctgcacctgatcaagcaccggg |
| by mRNA K | | gctacctgtcccagcggaagaacgagggcgagaccgccgacaaggagctgggcgccctgctgaagggcgtggccaacaacgcc |
| | | cacgccctgcagaccggcgacttccggacccccgccgagctggccctgaacaagttcgagaaggagtccggccacatccggaa |
| | | ccagcggggcgactactcccacaccttctcccggaaggacctgcaggccgagctgatcctgctgttcgagaagcagaaggagt |
| | | tcggcaacccccacgtgtccggcggcctgaaggagggcatcgagaccctgctgatgacccagcggcccgccctgtccggcgac |
| | | gccgtgcagaagatgctgggccactgcaccttcgagcccgccgagcccaaggccgccaagaacacctacaccgccgagcggtt |
| | | catctggctgaccaagctgaacaacctgcggatcctggagcagggctccgagcggcccctgaccgacaccgagcgggccaccc |
| | | tgatggacgagccctaccggaagtccaagctgacctacgcccaggcccggaagctgctgggcctggaggacaccgccttcttc |
| | | aagggcctgcggtacggcaaggacaacgccgaggcctccaccctgatggagatgaaggcctaccacgccatctcccgggccct |
| | | ggagaaggagggcctgaaggacaagaagtcccccctgaacctgtcctccgagctgcaggacgagatcggcaccgccttctccc |
| | | tgttcaagaccgacgaggacatcaccggccggctgaaggaccgggtgcagcccgagatcctggaggccctgctgaagcacatc |
| | | tccttcgacaagttcgtgcagatctccctgaaggccctgcggcggatcgtgcccctgatggagcagggcaagcggtacgacga |
| | | ggcctgcgccgagatctacggcgaccactacggcaagaagaacaccgaggagaagatctacctgccccccatccccgccgacg |
| | | agatccggaaccccgtggtgctgcgggccctgtcccaggcccggaaggtgatcaacggcgtggtgcggcggtacggctccccc |
| | | gcccggatccacatcgagaccgcccgggaggtgggcaagtccttcaaggaccggaaggagatcgagaagcggcaggaggagaa |
| | | ccggaaggaccgggagaaggccgccgccaagttccgggagtacttccccaacttcgtgggcgagcccaagtccaaggacatcc |
| | | tgaagctgcggctgtacgagcagcagcacggcaagtgcctgtactccggcaaggagatcaacctggtgcggctgaacgagaag |
| | | ggctacgtggagatcgaccacgccctgcccttctcccggacctgggacgactccttcaacaacaaggtgctggtgctgggctc |
| | | cgagaaccagaacaagggcaaccagaccccctacgagtacttcaacggcaaggacaactcccgggagtggcaggagttcaagg |
| | | cccgggtggagacctcccggttcccccggtccaagaagcagcggatcctgctgcagaagttcgacgaggacggcttcaaggag |
| | | tgcaacctgaacgacacccggtacgtgaaccggttcctgtgccagttcgtggccgaccacatcctgctgaccggcaagggcaa |
| | | gcggcgggtgttcgcctccaacggccagatcaccaacctgctgcggggcttctggggcctgcggaaggtgcgggccgagaacg |
| | | accggcaccacgccctggacgccgtggtggtggcctgctccaccgtggccatgcagcagaagatcacccggttcgtgcggtac |
| | | aaggagatgaacgccttcgacggcaagaccatcgacaaggagaccggcaaggtgctgcaccagaagacccacttcccccagcc |
| | | ctgggagttcttcgcccaggaggtgatgatccgggtgttcggcaagcccgacggcaagcccgagttcgaggaggccgacaccc |
| | | ccgagaagctgcggaccctgctggccgagaagctgtcctcccggcccgaggccgtgcacgagtacgtgacccccctgttcgtg |
| | | tcccgggcccccaaccggaagatgtccggcgcccacaaggacaccctgcggtccgccaagcggttcgtgaagcacaacgagaa |
| | | gatctccgtgaagcgggtgtggctgaccgagatcaagctggccgacctggagaacatggtgaactacaagaacggccgggaga |
| | | tcgagctgtacgaggccctgaaggcccggctggaggcctacggcggcaacgccaagcaggccttcgaccccaaggacaacccc |
| | | ttctacaagaagggcggccagctggtgaaggccgtgcgggtggagaagacccaggagtccggcgtgctgctgaacaagaagaa |
| | | cgcctacaccatcgccgacaacggcgacatggtgcgggtggacgtgttctgcaaggtggacaagaagggcaagaaccagtact |
| | | tcatcgtgcccatctacgcctggcaggtggccgagaacatcctgcccgacatcgactgcaagggctaccggatcgacgactcc |
| | | tacaccttctgcttctccctgcacaagtacgacctgatcgccttccagaaggacgagaagtccaaggtggagttcgcctacta |
| | | catcaactgcgactcctccaacggccggttctacctggcctggcacgacaagggctccaaggagcagcagttccggatctcca |
| | | cccagaacctggtgctgatccagaagtaccaggtgaacgagctgggcaaggagatccggccctgccggctgaagaagcggccc |
| | | cccgtgcgg |
| Open | 36 | atgGACGGCTCCGGCGGCGGCTCCCCCAAGAAGAAGCGGAAGGTGGGCGGCTCCGGCGGCGGCgccgccttcaagcccaaccc |
| reading | | catcaactacatcctgggcctggacatcggcatcgcctccgtgggctgggccatggtggagatcgacgaggaggagaacccca |
| frame for | | tccggctgatcgacctgggcgtgcgggtgttcgagcgggccgaggtgcccaagaccggcgactccctggccatggcccggcgg |
| Nme2Cas9 | | ctggcccggtccgtgcggcggctgacccggcggcgggcccaccggctgctgcgggcccggcggctgctgaagcgggagggcgt |
| encoded | | gctgcaggccgccgacttcgacgagaacggcctgatcaagtccctgcccaacaccccctggcagctgcgggccgccgccctgg |
| by mRNA L | | accggaagctgacccccctggagtggtccgccgtgctgctgcacctgatcaagcaccggggctacctgtcccagcggaagaac |
| | | gagggcgagaccgccgacaaggagctgggcgccctgctgaagggcgtggccaacaacgcccacgccctgcagaccggcgactt |
| | | ccggacccccgccgagctggccctgaacaagttcgagaaggagtccggccacatccggaaccagcggggcgactactcccaca |
| | | ccttctcccggaaggacctgcaggccgagctgatcctgctgttcgagaagcagaaggagttcggcaacccccacgtgtccggc |
| | | ggcctgaaggagggcatcgagaccctgctgatgacccagcggcccgccctgtccggcgacgccgtgcagaagatgctgggcca |
| | | ctgcaccttcgagcccgccgagcccaaggccgccaagaacacctacaccgccgagcggttcatctggctgaccaagctgaaca |
| | | acctgcggatcctggagcagggctccgagcggcccctgaccgacaccgagcgggccaccctgatggacgagccctaccggaag |
| | | tccaagctgacctacgcccaggcccggaagctgctgggcctggaggacaccgccttcttcaagggcctgcggtacggcaagga |
| | | caacgccgaggcctccaccctgatggagatgaaggcctaccacgccatctcccgggccctggagaaggagggcctgaaggaca |
| | | agaagtcccccctgaacctgtcctccgagctgcaggacgagatcggcaccgccttctccctgttcaagaccgacgaggacatc |
| | | accggccggctgaaggaccgggtgcagcccgagatcctggaggccctgctgaagcacatctccttcgacaagttcgtgcagat |
| | | ctccctgaaggccctgcggcggatcgtgcccctgatggagcagggcaagcggtacgacgaggcctgcgccgagatctacggcg |
| | | accactacggcaagaagaacaccgaggagaagatctacctgccccccatccccgccgacgagatccggaaccccgtggtgctg |
| | | cgggccctgtcccaggcccggaaggtgatcaacggcgtggtgcggcggtacggctcccccgcccggatccacatcgagaccgc |
| | | ccgggaggtgggcaagtccttcaaggaccggaaggagatcgagaagcggcaggaggagaaccggaaggaccgggagaaggccg |
| | | ccgccaagttccgggagtacttccccaacttcgtgggcgagcccaagtccaaggacatcctgaagctgcggctgtacgagcag |
| | | cagcacggcaagtgcctgtactccggcaaggagatcaacctggtgcggctgaacgagaagggctacgtggagatcgaccacgc |
| | | cctgcccttctcccggacctgggacgactccttcaacaacaaggtgctggtgctgggctccgagaaccagaacaagggcaacc |
| | | agaccccctacgagtacttcaacggcaaggacaactcccgggagtggcaggagttcaaggcccgggtggagacctcccggttc |
| | | ccccggtccaagaagcagcggatcctgctgcagaagttcgacgaggacggcttcaaggagtgcaacctgaacgacacccggta |
| | | cgtgaaccggttcctgtgccagttcgtggccgaccacatcctgctgaccggcaagggcaagcggcgggtgttcgcctccaacg |
| | | gccagatcaccaacctgctgcggggcttctggggcctgcggaaggtgcgggccgagaacgaccggcaccacgccctggacgcc |
| | | gtggtggtggcctgctccaccgtggccatgcagcagaagatcacccggttcgtgcggtacaaggagatgaacgccttcgacgg |
| | | caagaccatcgacaaggagaccggcaaggtgctgcaccagaagacccacttcccccagccctgggagttcttcgcccaggagg |
| | | tgatgatccgggtgttcggcaagcccgacggcaagcccgagttcgaggaggccgacacccccgagaagctgcggaccctgctg |
| | | gccgagaagctgtcctcccggcccgaggccgtgcacgagtacgtgacccccctgttcgtgtcccgggcccccaaccggaagat |
| | | gtccggcgcccacaaggacaccctgcggtccgccaagcggttcgtgaagcacaacgagaagatctccgtgaagcgggtgtggc |
| | | tgaccgagatcaagctggccgacctggagaacatggtgaactacaagaacggccgggagatcgagctgtacgaggccctgaag |
| | | gcccggctggaggcctacggcggcaacgccaagcaggccttcgaccccaaggacaaccccttctacaagaagggcggccagct |
| | | ggtgaaggccgtgcgggtggagaagacccaggagtccggcgtgctgctgaacaagaagaacgcctacaccatcgccgacaacg |
| | | gcgacatggtgcgggtggacgtgttctgcaaggtggacaagaagggcaagaaccagtacttcatcgtgcccatctacgcctgg |
| | | caggtggccgagaacatcctgcccgacatcgactgcaagggctaccggatcgacgactcctacaccttctgcttctccctgca |
| | | caagtacgacctgatcgccttccagaaggacgagaagtccaaggtggagttcgcctactacatcaactgcgactcctccaacg |
| | | gccggttctacctggcctggcacgacaagggctccaaggagcagcagttccggatctccacccagaacctggtgctgatccag |
| | | aagtaccaggtgaacgagctgggcaaggagatccggccctgccggctgaagaagcggccccccgtgcggtag |
| Open | 37 | ATGGACGGCTCCGGCGGCGGCTCCCCCAAGAAGAAGCGGAAGGTGGGCGGCTCCGGCGGCGGCGCCGCCTTCAAGCCCAACCC |
| reading | | CATCAACTACATCCTGGGCCTGGACATCGGCATCGCCTCCGTGGGCTGGGCCATGGTGGAGATCGACGAGGAGGAGAACCCCA |
| frame for | | TCCGGCTGATCGACCTGGGCGTGCGGGTGTTCGAGCGGGCCGAGGTGCCCAAGACCGGCGACTCCCTGGCCATGGCCCGGCGG |
| Nme2Cas9 | | CTGGCCCGGTCCGTGCGGCGGCTGACCCGGCGGCGGGCCCACCGGCTGCTGCGGGCCCGGCGGCTGCTGAAGCGGGAGGGCGT |
| with | | GCTGCAGGCCGCCGACTTCGACGAGAACGGCCTGATCAAGTCCCTGCCCAACACCCCCTGGCAGCTGCGGGCCGCCGCCCTGG |
| HiBiT tag | | ACCGGAAGCTGACCCCCCTGGAGTGGTCCGCCGTGCTGCTGCACCTGATCAAGCACCGGGGCTACCTGTCCCAGCGGAAGAAC |
| encoded | | GAGGGCGAGACCGCCGACAAGGAGCTGGGCGCCCTGCTGAAGGGCGTGGCCAACAACGCCCACGCCCTGCAGACCGGCGACTT |
| by mRNA M | | CCGGACCCCCGCCGAGCTGGCCCTGAACAAGTTCGAGAAGGAGTCCGGCCACATCCGGAACCAGCGGGGCGACTACTCCCACA |
| | | CCTTCTCCCGGAAGGACCTGCAGGCCGAGCTGATCCTGCTGTTCGAGAAGCAGAAGGAGTTCGGCAACCCCCACGTGTCCGGC |
| | | GGCCTGAAGGAGGGCATCGAGACCCTGCTGATGACCCAGCGGCCCGCCCTGTCCGGCGACGCCGTGCAGAAGATGCTGGGCCA |
| | | CTGCACCTTCGAGCCCGCCGAGCCCAAGGCCGCCAAGAACACCTACACCGCCGAGCGGTTCATCTGGCTGACCAAGCTGAACA |
| | | ACCTGCGGATCCTGGAGCAGGGCTCCGAGCGGCCCCTGACCGACACCGAGCGGGCCACCCTGATGGACGAGCCCTACCGGAAG |
| | | TCCAAGCTGACCTACGCCCAGGCCCGGAAGCTGCTGGGCCTGGAGGACACCGCCTTCTTCAAGGGCCTGCGGTACGGCAAGGA |
| | | CAACGCCGAGGCCTCCACCCTGATGGAGATGAAGGCCTACCACGCCATCTCCCGGGCCCTGGAGAAGGAGGGCCTGAAGGACA |
| | | AGAAGTCCCCCCTGAACCTGTCCTCCGAGCTGCAGGACGAGATCGGCACCGCCTTCTCCCTGTTCAAGACCGACGAGGACATC |
| | | ACCGGCCGGCTGAAGGACCGGGTGCAGCCCGAGATCCTGGAGGCCCTGCTGAAGCACATCTCCTTCGACAAGTTCGTGCAGAT |
| | | CTCCCTGAAGGCCCTGCGGCGGATCGTGCCCCTGATGGAGCAGGGCAAGCGGTACGACGAGGCCTGCGCCGAGATCTACGGCG |
| | | ACCACTACGGCAAGAAGAACACCGAGGAGAAGATCTACCTGCCCCCCATCCCCGCCGACGAGATCCGGAACCCCGTGGTGCTG |
| | | CGGGCCCTGTCCCAGGCCCGGAAGGTGATCAACGGCGTGGTGCGGCGGTACGGCTCCCCCGCCCGGATCCACATCGAGACCGC |
| | | CCGGGAGGTGGGCAAGTCCTTCAAGGACCGGAAGGAGATCGAGAAGCGGCAGGAGGAGAACCGGAAGGACCGGGAGAAGGCCG |
| | | CCGCCAAGTTCCGGGAGTACTTCCCCAACTTCGTGGGCGAGCCCAAGTCCAAGGACATCCTGAAGCTGCGGCTGTACGAGCAG |
| | | CAGCACGGCAAGTGCCTGTACTCCGGCAAGGAGATCAACCTGGTGCGGCTGAACGAGAAGGGCTACGTGGAGATCGACCACGC |
| | | CCTGCCCTTCTCCCGGACCTGGGACGACTCCTTCAACAACAAGGTGCTGGTGCTGGGCTCCGAGAACCAGAACAAGGGCAACC |
| | | AGACCCCCTACGAGTACTTCAACGGCAAGGACAACTCCCGGGAGTGGCAGGAGTTCAAGGCCCGGGTGGAGACCTCCCGGTTC |
| | | CCCCGGTCCAAGAAGCAGCGGATCCTGCTGCAGAAGTTCGACGAGGACGGCTTCAAGGAGTGCAACCTGAACGACACCCGGTA |
| | | CGTGAACCGGTTCCTGTGCCAGTTCGTGGCCGACCACATCCTGCTGACCGGCAAGGGCAAGCGGCGGGTGTTCGCCTCCAACG |
| | | GCCAGATCACCAACCTGCTGCGGGGCTTCTGGGGCCTGCGGAAGGTGCGGGCCGAGAACGACCGGCACCACGCCCTGGACGCC |
| | | GTGGTGGTGGCCTGCTCCACCGTGGCCATGCAGCAGAAGATCACCCGGTTCGTGCGGTACAAGGAGATGAACGCCTTCGACGG |
| | | CAAGACCATCGACAAGGAGACCGGCAAGGTGCTGCACCAGAAGACCCACTTCCCCCAGCCCTGGGAGTTCTTCGCCCAGGAGG |
| | | TGATGATCCGGGTGTTCGGCAAGCCCGACGGCAAGCCCGAGTTCGAGGAGGCCGACACCCCCGAGAAGCTGCGGACCCTGCTG |
| | | GCCGAGAAGCTGTCCTCCCGGCCCGAGGCCGTGCACGAGTACGTGACCCCCCTGTTCGTGTCCCGGGCCCCCAACCGGAAGAT |
| | | GTCCGGCGCCCACAAGGACACCCTGCGGTCCGCCAAGCGGTTCGTGAAGCACAACGAGAAGATCTCCGTGAAGCGGGTGTGGC |
| | | TGACCGAGATCAAGCTGGCCGACCTGGAGAACATGGTGAACTACAAGAACGGCCGGGAGATCGAGCTGTACGAGGCCCTGAAG |
| | | GCCCGGCTGGAGGCCTACGGCGGCAACGCCAAGCAGGCCTTCGACCCCAAGGACAACCCCTTCTACAAGAAGGGCGGCCAGCT |
| | | GGTGAAGGCCGTGCGGGTGGAGAAGACCCAGGAGTCCGGCGTGCTGCTGAACAAGAAGAACGCCTACACCATCGCCGACAACG |
| | | GCGACATGGTGCGGGTGGACGTGTTCTGCAAGGTGGACAAGAAGGGCAAGAACCAGTACTTCATCGTGCCCATCTACGCCTGG |
| | | CAGGTGGCCGAGAACATCCTGCCCGACATCGACTGCAAGGGCTACCGGATCGACGACTCCTACACCTTCTGCTTCTCCCTGCA |
| | | CAAGTACGACCTGATCGCCTTCCAGAAGGACGAGAAGTCCAAGGTGGAGTTCGCCTACTACATCAACTGCGACTCCTCCAACG |
| | | GCCGGTTCTACCTGGCCTGGCACGACAAGGGCTCCAAGGAGCAGCAGTTCCGGATCTCCACCCAGAACCTGGTGCTGATCCAG |
| | | AAGTACCAGGTGAACGAGCTGGGCAAGGAGATCCGGCCCTGCCGGCTGAAGAAGCGGCCCCCCGTGCGGTCCGAGTCCGCCAC |
| | | CCCCGAGTCCGTGTCCGGCTGGCGGCTGTTCAAGAAGATCTCCTAG |
| Open | 38 | atgGACGGCTCCGGCGGCGGCTCCCCCAAGAAGAAGCGGAAGGTGGGCGGCTCCGGCGGCGGCgccgccttcaagcccaaccc |
| reading | | catcaactacatcctgggcctggacatcggcatcgcctccgtgggctgggccatggtggagatcgacgaggaggagaacccca |
| frame for | | tccggctgatcgacctgggcgtgcgggtgttcgagcgggccgaggtgcccaagaccggcgactccctggccatggcccggcgg |
| Nme2Cas9 | | ctggcccggtccgtgcggcggctgacccggcggcgggcccaccggctgctgcgggcccggcggctgctgaagcgggagggcgt |
| encoded | | gctgcaggccgccgacttcgacgagaacggcctgatcaagtccctgcccaacaccccctggcagctgcgggccgccgccctgg |
| by mRNA N | | accggaagctgacccccctggagtggtccgccgtgctgctgcacctgatcaagcaccggggctacctgtcccagcggaagaac |
| | | gagggcgagaccgccgacaaggagctgggcgccctgctgaagggcgtggccaacaacgcccacgccctgcagaccggcgactt |
| | | ccggacccccgccgagctggccctgaacaagttcgagaaggagtccggccacatccggaaccagcggggcgactactcccaca |
| | | ccttctcccggaaggacctgcaggccgagctgatcctgctgttcgagaagcagaaggagttcggcaacccccacgtgtccggc |
| | | ggcctgaaggagggcatcgagaccctgctgatgacccagcggcccgccctgtccggcgacgccgtgcagaagatgctgggcca |
| | | ctgcaccttcgagcccgccgagcccaaggccgccaagaacacctacaccgccgagcggttcatctggctgaccaagctgaaca |
| | | acctgcggatcctggagcagggctccgagcggcccctgaccgacaccgagcgggccaccctgatggacgagccctaccggaag |
| | | tccaagctgacctacgcccaggcccggaagctgctgggcctggaggacaccgccttcttcaagggcctgcggtacggcaagga |
| | | caacgccgaggcctccaccctgatggagatgaaggcctaccacgccatctcccgggccctggagaaggagggcctgaaggaca |
| | | agaagtcccccctgaacctgtcctccgagctgcaggacgagatcggcaccgccttctccctgttcaagaccgacgaggacatc |
| | | accggccggctgaaggaccgggtgcagcccgagatcctggaggccctgctgaagcacatctccttcgacaagttcgtgcagat |
| | | ctccctgaaggccctgcggcggatcgtgcccctgatggagcagggcaagcggtacgacgaggcctgcgccgagatctacggcg |
| | | accactacggcaagaagaacaccgaggagaagatctacctgccccccatccccgccgacgagatccggaaccccgtggtgctg |
| | | cgggccctgtcccaggcccggaaggtgatcaacggcgtggtgcggcggtacggctcccccgcccggatccacatcgagaccgc |
| | | ccgggaggtgggcaagtccttcaaggaccggaaggagatcgagaagcggcaggaggagaaccggaaggaccgggagaaggccg |
| | | ccgccaagttccgggagtacttccccaacttcgtgggcgagcccaagtccaaggacatcctgaagctgcggctgtacgagcag |
| | | cagcacggcaagtgcctgtactccggcaaggagatcaacctggtgcggctgaacgagaagggctacgtggagatcgaccacgc |
| | | cctgcccttctcccggacctgggacgactccttcaacaacaaggtgctggtgctgggctccgagaaccagaacaagggcaacc |
| | | agaccccctacgagtacttcaacggcaaggacaactcccgggagtggcaggagttcaaggcccgggtggagacctcccggttc |
| | | ccccggtccaagaagcagcggatcctgctgcagaagttcgacgaggacggcttcaaggagtgcaacctgaacgacacccggta |
| | | cgtgaaccggttcctgtgccagttcgtggccgaccacatcctgctgaccggcaagggcaagcggcgggtgttcgcctccaacg |
| | | gccagatcaccaacctgctgcggggcttctggggcctgcggaaggtgcgggccgagaacgaccggcaccacgccctggacgcc |
| | | gtggtggtggcctgctccaccgtggccatgcagcagaagatcacccggttcgtgcggtacaaggagatgaacgccttcgacgg |
| | | caagaccatcgacaaggagaccggcaaggtgctgcaccagaagacccacttcccccagccctgggagttcttcgcccaggagg |
| | | tgatgatccgggtgttcggcaagcccgacggcaagcccgagttcgaggaggccgacacccccgagaagctgcggaccctgctg |
| | | gccgagaagctgtcctcccggcccgaggccgtgcacgagtacgtgacccccctgttcgtgtcccgggcccccaaccggaagat |
| | | gtccggcgcccacaaggacaccctgcggtccgccaagcggttcgtgaagcacaacgagaagatctccgtgaagcgggtgtggc |
| | | tgaccgagatcaagctggccgacctggagaacatggtgaactacaagaacggccgggagatcgagctgtacgaggccctgaag |
| | | gcccggctggaggcctacggcggcaacgccaagcaggccttcgaccccaaggacaaccccttctacaagaagggcggccagct |
| | | ggtgaaggccgtgcgggtggagaagacccaggagtccggcgtgctgctgaacaagaagaacgcctacaccatcgccgacaacg |
| | | gcgacatggtgcgggtggacgtgttctgcaaggtggacaagaagggcaagaaccagtacttcatcgtgcccatctacgcctgg |
| | | caggtggccgagaacatcctgcccgacatcgactgcaagggctaccggatcgacgactcctacaccttctgcttctccctgca |
| | | caagtacgacctgatcgccttccagaaggacgagaagtccaaggtggagttcgcctactacatcaactgcgactcctccaacg |
| | | gccggttctacctggcctggcacgacaagggctccaaggagcagcagttccggatctccacccagaacctggtgctgatccag |
| | | aagtaccaggtgaacgagctgggcaaggagatccggccctgccggctgaagaagcggccccccgtgcggTCCGGAAAGCGGAC |
| | | CGCCGACGGCTCCGGAGGAGGAAGCCCCGCCGCCAAGAAGAAGAAGCTGGACtag |
| Open | 39 | atgGACGGCTCCGGCGGCGGCTCCCCCAAGAAGAAGCGGAAGGTGGAGGACAAGCGGCCCGCCGCCACCAAGAAGGCCGGCCA |
| reading | | GGCCAAGAAGAAGAAGGGCGGCTCCGGCGGCGGCgccgccttcaagcccaaccccatcaactacatcctgggcctggacatcg |
| frame for | | gcatcgcctccgtgggctgggccatggtggagatcgacgaggaggagaaccccatccggctgatcgacctgggcgtgcgggtg |
| Nme2Cas9 | | ttcgagcgggccgaggtgcccaagaccggcgactccctggccatggcccggcggctggcccggtccgtgcggcggctgacccg |
| encoded | | gcggcgggcccaccggctgctgcgggcccggcggctgctgaagcgggagggcgtgctgcaggccgccgacttcgacgagaacg |
| by mRNA O | | gcctgatcaagtccctgcccaacaccccctggcagctgcgggccgccgccctggaccggaagctgacccccctggagtggtcc |
| | | gccgtgctgctgcacctgatcaagcaccggggctacctgtcccagcggaagaacgagggcgagaccgccgacaaggagctggg |
| | | cgccctgctgaagggcgtggccaacaacgcccacgccctgcagaccggcgacttccggacccccgccgagctggccctgaaca |
| | | agttcgagaaggagtccggccacatccggaaccagcggggcgactactcccacaccttctcccggaaggacctgcaggccgag |
| | | ctgatcctgctgttcgagaagcagaaggagttcggcaacccccacgtgtccggcggcctgaaggagggcatcgagaccctgct |
| | | gatgacccagcggcccgccctgtccggcgacgccgtgcagaagatgctgggccactgcaccttcgagcccgccgagcccaagg |
| | | ccgccaagaacacctacaccgccgagcggttcatctggctgaccaagctgaacaacctgcggatcctggagcagggctccgag |
| | | cggcccctgaccgacaccgagcgggccaccctgatggacgagccctaccggaagtccaagctgacctacgcccaggcccggaa |
| | | gctgctgggcctggaggacaccgccttcttcaagggcctgcggtacggcaaggacaacgccgaggcctccaccctgatggaga |
| | | tgaaggcctaccacgccatctcccgggccctggagaaggagggcctgaaggacaagaagtcccccctgaacctgtcctccgag |
| | | ctgcaggacgagatcggcaccgccttctccctgttcaagaccgacgaggacatcaccggccggctgaaggaccgggtgcagcc |
| | | cgagatcctggaggccctgctgaagcacatctccttcgacaagttcgtgcagatctccctgaaggccctgcggcggatcgtgc |
| | | ccctgatggagcagggcaagcggtacgacgaggcctgcgccgagatctacggcgaccactacggcaagaagaacaccgaggag |
| | | aagatctacctgccccccatccccgccgacgagatccggaaccccgtggtgctgcgggccctgtcccaggcccggaaggtgat |
| | | caacggcgtggtgcggcggtacggctcccccgcccggatccacatcgagaccgcccgggaggtgggcaagtccttcaaggacc |
| | | ggaaggagatcgagaagcggcaggaggagaaccggaaggaccgggagaaggccgccgccaagttccgggagtacttccccaac |
| | | ttcgtgggcgagcccaagtccaaggacatcctgaagctgcggctgtacgagcagcagcacggcaagtgcctgtactccggcaa |
| | | ggagatcaacctggtgcggctgaacgagaagggctacgtggagatcgaccacgccctgcccttctcccggacctgggacgact |
| | | ccttcaacaacaaggtgctggtgctgggctccgagaaccagaacaagggcaaccagaccccctacgagtacttcaacggcaag |
| | | gacaactcccgggagtggcaggagttcaaggcccgggtggagacctcccggttcccccggtccaagaagcagcggatcctgct |
| | | gcagaagttcgacgaggacggcttcaaggagtgcaacctgaacgacacccggtacgtgaaccggttcctgtgccagttcgtgg |
| | | ccgaccacatcctgctgaccggcaagggcaagcggcgggtgttcgcctccaacggccagatcaccaacctgctgcggggcttc |
| | | tggggcctgcggaaggtgcgggccgagaacgaccggcaccacgccctggacgccgtggtggtggcctgctccaccgtggccat |
| | | gcagcagaagatcacccggttcgtgcggtacaaggagatgaacgccttcgacggcaagaccatcgacaaggagaccggcaagg |
| | | tgctgcaccagaagacccacttcccccagccctgggagttcttcgcccaggaggtgatgatccgggtgttcggcaagcccgac |
| | | ggcaagcccgagttcgaggaggccgacacccccgagaagctgcggaccctgctggccgagaagctgtcctcccggcccgaggc |
| | | cgtgcacgagtacgtgacccccctgttcgtgtcccgggcccccaaccggaagatgtccggcgcccacaaggacaccctgcggt |
| | | ccgccaagcggttcgtgaagcacaacgagaagatctccgtgaagcgggtgtggctgaccgagatcaagctggccgacctggag |
| | | aacatggtgaactacaagaacggccgggagatcgagctgtacgaggccctgaaggcccggctggaggcctacggcggcaacgc |
| | | caagcaggccttcgaccccaaggacaaccccttctacaagaagggcggccagctggtgaaggccgtgcgggtggagaagaccc |
| | | aggagtccggcgtgctgctgaacaagaagaacgcctacaccatcgccgacaacggcgacatggtgcgggtggacgtgttctgc |
| | | aaggtggacaagaagggcaagaaccagtacttcatcgtgcccatctacgcctggcaggtggccgagaacatcctgcccgacat |
| | | cgactgcaagggctaccggatcgacgactcctacaccttctgcttctccctgcacaagtacgacctgatcgccttccagaagg |
| | | acgagaagtccaaggtggagttcgcctactacatcaactgcgactcctccaacggccggttctacctggcctggcacgacaag |
| | | ggctccaaggagcagcagttccggatctccacccagaacctggtgctgatccagaagtaccaggtgaacgagctgggcaagga |
| | | gatccggccctgccggctgaagaagcggccccccgtgcggtag |
| Open | 40 | atgGACGGCTCCGGCGGCGGCTCCCCCAAGAAGAAGCGGAAGGTGGAGGACAAGCGGCCCGCCGCCACCAAGAAGGCCGGCCA |
| reading | | GGCCAAGAAGAAGAAGGGCGGCTCCGGCGGCGGCGCCGCCTTCAAGCCCAACCCCATCAACTACATCCTGGGCCTGGACATCG |
| frame for | | GCATCGCCTCCGTGGGCTGGGCCATGGTGGAGATCGACGAGGAGGAGAACCCCATCCGGCTGATCGACCTGGGCGTGCGGGTG |
| Nme2Cas9 | | TTCGAGCGGGCCGAGGTGCCCAAGACCGGCGACTCCCTGGCCATGGCCCGGCGGCTGGCCCGGTCCGTGCGGCGGCTGACCCG |
| with | | GCGGCGGGCCCACCGGCTGCTGCGGGCCCGGCGGCTGCTGAAGCGGGAGGGCGTGCTGCAGGCCGCCGACTTCGACGAGAACG |
| HiBiT tag | | GCCTGATCAAGTCCCTGCCCAACACCCCCTGGCAGCTGCGGGCCGCCGCCCTGGACCGGAAGCTGACCCCCCTGGAGTGGTCC |
| encoded | | GCCGTGCTGCTGCACCTGATCAAGCACCGGGGCTACCTGTCCCAGCGGAAGAACGAGGGCGAGACCGCCGACAAGGAGCTGGG |
| by mRNA P | | CGCCCTGCTGAAGGGCGTGGCCAACAACGCCCACGCCCTGCAGACCGGCGACTTCCGGACCCCCGCCGAGCTGGCCCTGAACA |
| | | AGTTCGAGAAGGAGTCCGGCCACATCCGGAACCAGCGGGGCGACTACTCCCACACCTTCTCCCGGAAGGACCTGCAGGCCGAG |
| | | CTGATCCTGCTGTTCGAGAAGCAGAAGGAGTTCGGCAACCCCCACGTGTCCGGCGGCCTGAAGGAGGGCATCGAGACCCTGCT |
| | | GATGACCCAGCGGCCCGCCCTGTCCGGCGACGCCGTGCAGAAGATGCTGGGCCACTGCACCTTCGAGCCCGCCGAGCCCAAGG |
| | | CCGCCAAGAACACCTACACCGCCGAGCGGTTCATCTGGCTGACCAAGCTGAACAACCTGCGGATCCTGGAGCAGGGCTCCGAG |
| | | CGGCCCCTGACCGACACCGAGCGGGCCACCCTGATGGACGAGCCCTACCGGAAGTCCAAGCTGACCTACGCCCAGGCCCGGAA |
| | | GCTGCTGGGCCTGGAGGACACCGCCTTCTTCAAGGGCCTGCGGTACGGCAAGGACAACGCCGAGGCCTCCACCCTGATGGAGA |
| | | TGAAGGCCTACCACGCCATCTCCCGGGCCCTGGAGAAGGAGGGCCTGAAGGACAAGAAGTCCCCCCTGAACCTGTCCTCCGAG |
| | | CTGCAGGACGAGATCGGCACCGCCTTCTCCCTGTTCAAGACCGACGAGGACATCACCGGCCGGCTGAAGGACCGGGTGCAGCC |
| | | CGAGATCCTGGAGGCCCTGCTGAAGCACATCTCCTTCGACAAGTTCGTGCAGATCTCCCTGAAGGCCCTGCGGCGGATCGTGC |
| | | CCCTGATGGAGCAGGGCAAGCGGTACGACGAGGCCTGCGCCGAGATCTACGGCGACCACTACGGCAAGAAGAACACCGAGGAG |
| | | AAGATCTACCTGCCCCCCATCCCCGCCGACGAGATCCGGAACCCCGTGGTGCTGCGGGCCCTGTCCCAGGCCCGGAAGGTGAT |
| | | CAACGGCGTGGTGCGGCGGTACGGCTCCCCCGCCCGGATCCACATCGAGACCGCCCGGGAGGTGGGCAAGTCCTTCAAGGACC |
| | | GGAAGGAGATCGAGAAGCGGCAGGAGGAGAACCGGAAGGACCGGGAGAAGGCCGCCGCCAAGTTCCGGGAGTACTTCCCCAAC |
| | | TTCGTGGGCGAGCCCAAGTCCAAGGACATCCTGAAGCTGCGGCTGTACGAGCAGCAGCACGGCAAGTGCCTGTACTCCGGCAA |
| | | GGAGATCAACCTGGTGCGGCTGAACGAGAAGGGCTACGTGGAGATCGACCACGCCCTGCCCTTCTCCCGGACCTGGGACGACT |
| | | CCTTCAACAACAAGGTGCTGGTGCTGGGCTCCGAGAACCAGAACAAGGGCAACCAGACCCCCTACGAGTACTTCAACGGCAAG |
| | | GACAACTCCCGGGAGTGGCAGGAGTTCAAGGCCCGGGTGGAGACCTCCCGGTTCCCCCGGTCCAAGAAGCAGCGGATCCTGCT |
| | | GCAGAAGTTCGACGAGGACGGCTTCAAGGAGTGCAACCTGAACGACACCCGGTACGTGAACCGGTTCCTGTGCCAGTTCGTGG |
| | | CCGACCACATCCTGCTGACCGGCAAGGGCAAGCGGCGGGTGTTCGCCTCCAACGGCCAGATCACCAACCTGCTGCGGGGCTTC |
| | | TGGGGCCTGCGGAAGGTGCGGGCCGAGAACGACCGGCACCACGCCCTGGACGCCGTGGTGGTGGCCTGCTCCACCGTGGCCAT |
| | | GCAGCAGAAGATCACCCGGTTCGTGCGGTACAAGGAGATGAACGCCTTCGACGGCAAGACCATCGACAAGGAGACCGGCAAGG |
| | | TGCTGCACCAGAAGACCCACTTCCCCCAGCCCTGGGAGTTCTTCGCCCAGGAGGTGATGATCCGGGTGTTCGGCAAGCCCGAC |
| | | GGCAAGCCCGAGTTCGAGGAGGCCGACACCCCCGAGAAGCTGCGGACCCTGCTGGCCGAGAAGCTGTCCTCCCGGCCCGAGGC |
| | | CGTGCACGAGTACGTGACCCCCCTGTTCGTGTCCCGGGCCCCCAACCGGAAGATGTCCGGCGCCCACAAGGACACCCTGCGGT |
| | | CCGCCAAGCGGTTCGTGAAGCACAACGAGAAGATCTCCGTGAAGCGGGTGTGGCTGACCGAGATCAAGCTGGCCGACCTGGAG |
| | | AACATGGTGAACTACAAGAACGGCCGGGAGATCGAGCTGTACGAGGCCCTGAAGGCCCGGCTGGAGGCCTACGGCGGCAACGC |
| | | CAAGCAGGCCTTCGACCCCAAGGACAACCCCTTCTACAAGAAGGGCGGCCAGCTGGTGAAGGCCGTGCGGGTGGAGAAGACCC |
| | | AGGAGTCCGGCGTGCTGCTGAACAAGAAGAACGCCTACACCATCGCCGACAACGGCGACATGGTGCGGGTGGACGTGTTCTGC |
| | | AAGGTGGACAAGAAGGGCAAGAACCAGTACTTCATCGTGCCCATCTACGCCTGGCAGGTGGCCGAGAACATCCTGCCCGACAT |
| | | CGACTGCAAGGGCTACCGGATCGACGACTCCTACACCTTCTGCTTCTCCCTGCACAAGTACGACCTGATCGCCTTCCAGAAGG |
| | | ACGAGAAGTCCAAGGTGGAGTTCGCCTACTACATCAACTGCGACTCCTCCAACGGCCGGTTCTACCTGGCCTGGCACGACAAG |
| | | GGCTCCAAGGAGCAGCAGTTCCGGATCTCCACCCAGAACCTGGTGCTGATCCAGAAGTACCAGGTGAACGAGCTGGGCAAGGA |
| | | GATCCGGCCCTGCCGGCTGAAGAAGCGGCCCCCCGTGCGGTCCGAGTCCGCCACCCCCGAGTCCGTGTCCGGCTGGCGGCTGT |
| | | TCAAGAAGATCTCCTAG |
| Open | 41 | atgGACGGCTCCGGCGGCGGCTCCGAGGACAAGCGGCCCGCCGCCACCAAGAAGGCCGGCCAGGCCAAGAAGAAGAAGGGCGG |
| reading | | CTCCGGCGGCGGCgccgccttcaagcccaaccccatcaactacatcctgggcctggacatcggcatcgcctccgtgggctggg |
| frame for | | ccatggtggagatcgacgaggaggagaaccccatccggctgatcgacctgggcgtgcgggtgttcgagcgggccgaggtgccc |
| Nme2Cas9 | | aagaccggcgactccctggccatggcccggcggctggcccggtccgtgcggcggctgacccggcggcgggcccaccggctgct |
| encoded | | gcgggcccggcggctgctgaagcgggagggcgtgctgcaggccgccgacttcgacgagaacggcctgatcaagtccctgccca |
| by mRNA Q | | acaccccctggcagctgcgggccgccgccctggaccggaagctgacccccctggagtggtccgccgtgctgctgcacctgatc |
| | | aagcaccggggctacctgtcccagcggaagaacgagggcgagaccgccgacaaggagctgggcgccctgctgaagggcgtggc |
| | | caacaacgcccacgccctgcagaccggcgacttccggacccccgccgagctggccctgaacaagttcgagaaggagtccggcc |
| | | acatccggaaccagcggggcgactactcccacaccttctcccggaaggacctgcaggccgagctgatcctgctgttcgagaag |
| | | cagaaggagttcggcaacccccacgtgtccggcggcctgaaggagggcatcgagaccctgctgatgacccagcggcccgccct |
| | | gtccggcgacgccgtgcagaagatgctgggccactgcaccttcgagcccgccgagcccaaggccgccaagaacacctacaccg |
| | | ccgagcggttcatctggctgaccaagctgaacaacctgcggatcctggagcagggctccgagcggcccctgaccgacaccgag |
| | | cgggccaccctgatggacgagccctaccggaagtccaagctgacctacgcccaggcccggaagctgctgggcctggaggacac |
| | | cgccttcttcaagggcctgcggtacggcaaggacaacgccgaggcctccaccctgatggagatgaaggcctaccacgccatct |
| | | cccgggccctggagaaggagggcctgaaggacaagaagtcccccctgaacctgtcctccgagctgcaggacgagatcggcacc |
| | | gccttctccctgttcaagaccgacgaggacatcaccggccggctgaaggaccgggtgcagcccgagatcctggaggccctgct |
| | | gaagcacatctccttcgacaagttcgtgcagatctccctgaaggccctgcggcggatcgtgcccctgatggagcagggcaagc |
| | | ggtacgacgaggcctgcgccgagatctacggcgaccactacggcaagaagaacaccgaggagaagatctacctgccccccatc |
| | | cccgccgacgagatccggaaccccgtggtgctgcgggccctgtcccaggcccggaaggtgatcaacggcgtggtgcggcggta |
| | | cggctcccccgcccggatccacatcgagaccgcccgggaggtgggcaagtccttcaaggaccggaaggagatcgagaagcggc |
| | | aggaggagaaccggaaggaccgggagaaggccgccgccaagttccgggagtacttccccaacttcgtgggcgagcccaagtcc |
| | | aaggacatcctgaagctgcggctgtacgagcagcagcacggcaagtgcctgtactccggcaaggagatcaacctggtgcggct |
| | | gaacgagaagggctacgtggagatcgaccacgccctgcccttctcccggacctgggacgactccttcaacaacaaggtgctgg |
| | | tgctgggctccgagaaccagaacaagggcaaccagaccccctacgagtacttcaacggcaaggacaactcccgggagtggcag |
| | | gagttcaaggcccgggtggagacctcccggttcccccggtccaagaagcagcggatcctgctgcagaagttcgacgaggacgg |
| | | cttcaaggagtgcaacctgaacgacacccggtacgtgaaccggttcctgtgccagttcgtggccgaccacatcctgctgaccg |
| | | gcaagggcaagcggcgggtgttcgcctccaacggccagatcaccaacctgctgcggggcttctggggcctgcggaaggtgcgg |
| | | gccgagaacgaccggcaccacgccctggacgccgtggtggtggcctgctccaccgtggccatgcagcagaagatcacccggtt |
| | | cgtgcggtacaaggagatgaacgccttcgacggcaagaccatcgacaaggagaccggcaaggtgctgcaccagaagacccact |
| | | tcccccagccctgggagttcttcgcccaggaggtgatgatccgggtgttcggcaagcccgacggcaagcccgagttcgaggag |
| | | gccgacacccccgagaagctgcggaccctgctggccgagaagctgtcctcccggcccgaggccgtgcacgagtacgtgacccc |
| | | cctgttcgtgtcccgggcccccaaccggaagatgtccggcgcccacaaggacaccctgcggtccgccaagcggttcgtgaagc |
| | | acaacgagaagatctccgtgaagcgggtgtggctgaccgagatcaagctggccgacctggagaacatggtgaactacaagaac |
| | | ggccgggagatcgagctgtacgaggccctgaaggcccggctggaggcctacggcggcaacgccaagcaggccttcgaccccaa |
| | | ggacaaccccttctacaagaagggcggccagctggtgaaggccgtgcgggtggagaagacccaggagtccggcgtgctgctga |
| | | acaagaagaacgcctacaccatcgccgacaacggcgacatggtgcgggtggacgtgttctgcaaggtggacaagaagggcaag |
| | | aaccagtacttcatcgtgcccatctacgcctggcaggtggccgagaacatcctgcccgacatcgactgcaagggctaccggat |
| | | cgacgactcctacaccttctgcttctccctgcacaagtacgacctgatcgccttccagaaggacgagaagtccaaggtggagt |
| | | tcgcctactacatcaactgcgactcctccaacggccggttctacctggcctggcacgacaagggctccaaggagcagcagttc |
| | | cggatctccacccagaacctggtgctgatccagaagtaccaggtgaacgagctgggcaaggagatccggccctgccggctgaa |
| | | gaagcggccccccgtgcggtag |
| Open | 42 | ATGGACGGCTCCGGCGGCGGCTCCCCCAAGAAGAAGCGGAAGGTGGAGGACAAGCGGCCCGCCGCCACCAAGAAGGCCGGCCA |
| reading | | GGCCAAGAAGAAGAAGGGCGGCTCCGGCGGCGGCGAGGCCTCCCCCGCCTCCGGCCCCCGGCACCTGATGGACCCCCACATCT |
| frame for | | TCACCTCCAACTTCAACAACGGCATCGGCCGGCACAAGACCTACCTGTGCTACGAGGTGGAGCGGCTGGACAACGGCACCTCC |
| Nme2Cas9 | | GTGAAGATGGACCAGCACCGGGGCTTCCTGCACAACCAGGCCAAGAACCTGCTGTGCGGCTTCTACGGCCGGCACGCCGAGCT |
| base | | GCGGTTCCTGGACCTGGTGCCCTCCCTGCAGCTGGACCCCGCCCAGATCTACCGGGTGACCTGGTTCATCTCCTGGTCCCCCT |
| editor | | GCTTCTCCTGGGGCTGCGCCGGCGAGGTGCGGGCCTTCCTGCAGGAGAACACCCACGTGCGGCTGCGGATCTTCGCCGCCCGG |
| encoded | | ATCTACGACTACGACCCCCTGTACAAGGAGGCCCTGCAGATGCTGCGGGACGCCGGCGCCCAGGTGTCCATCATGACCTACGA |
| by mRNA R | | CGAGTTCAAGCACTGCTGGGACACCTTCGTGGACCACCAGGGCTGCCCCTTCCAGCCCTGGGACGGCCTGGACGAGCACTCCC |
| | | AGGCCCTGTCCGGCCGGCTGCGGGCCATCCTGCAGAACCAGGGCAACTCCGGCTCCGAGACCCCCGGCACCTCCGAGTCCGCC |
| | | ACCCCCGAGTCCGCAGCGTTCAAACCAAATcccatcaactacatcctgggcctggccatcggcatcgcctccgtgggctgggc |
| | | catggtggagatcgacgaggaggagaaccccatccggctgatcgacctgggcgtgcgggtgttcgagcgggccgaggtgccca |
| | | agaccggcgactccctggccatggcccggcggctggcccggtccgtgcggcggctgacccggcggcgggcccaccggctgctg |
| | | cgggcccggcggctgctgaagcgggagggcgtgctgcaggccgccgacttcgacgagaacggcctgatcaagtccctgcccaa |
| | | caccccctggcagctgcgggccgccgccctggaccggaagctgacccccctggagtggtccgccgtgctgctgcacctgatca |
| | | agcaccggggctacctgtcccagcggaagaacgagggcgagaccgccgacaaggagctgggcgccctgctgaagggcgtggcc |
| | | aacaacgcccacgccctgcagaccggcgacttccggacccccgccgagctggccctgaacaagttcgagaaggagtccggcca |
| | | catccggaaccagcggggcgactactcccacaccttctcccggaaggacctgcaggccgagctgatcctgctgttcgagaagc |
| | | agaaggagttcggcaacccccacgtgtccggcggcctgaaggagggcatcgagaccctgctgatgacccagcggcccgccctg |
| | | tccggcgacgccgtgcagaagatgctgggccactgcaccttcgagcccgccgagcccaaggccgccaagaacacctacaccgc |
| | | cgagcggttcatctggctgaccaagctgaacaacctgcggatcctggagcagggctccgagcggcccctgaccgacaccgagc |
| | | gggccaccctgatggacgagccctaccggaagtccaagctgacctacgcccaggcccggaagctgctgggcctggaggacacc |
| | | gccttcttcaagggcctgcggtacggcaaggacaacgccgaggcctccaccctgatggagatgaaggcctaccacgccatctc |
| | | ccgggccctggagaaggagggcctgaaggacaagaagtcccccctgaacctgtcctccgagctgcaggacgagatcggcaccg |
| | | ccttctccctgttcaagaccgacgaggacatcaccggccggctgaaggaccgggtgcagcccgagatcctggaggccctgctg |
| | | aagcacatctccttcgacaagttcgtgcagatctccctgaaggccctgcggcggatcgtgcccctgatggagcagggcaagcg |
| | | gtacgacgaggcctgcgccgagatctacggcgaccactacggcaagaagaacaccgaggagaagatctacctgccccccatcc |
| | | ccgccgacgagatccggaaccccgtggtgctgcgggccctgtcccaggcccggaaggtgatcaacggcgtggtgcggcggtac |
| | | ggctcccccgcccggatccacatcgagaccgcccgggaggtgggcaagtccttcaaggaccggaaggagatcgagaagcggca |
| | | ggaggagaaccggaaggaccgggagaaggccgccgccaagttccgggagtacttccccaacttcgtgggcgagcccaagtcca |
| | | aggacatcctgaagctgcggctgtacgagcagcagcacggcaagtgcctgtactccggcaaggagatcaacctggtgcggctg |
| | | aacgagaagggctacgtggagatcgaccacgccctgcccttctcccggacctgggacgactccttcaacaacaaggtgctggt |
| | | gctgggctccgagaaccagaacaagggcaaccagaccccctacgagtacttcaacggcaaggacaactcccgggagtggcagg |
| | | agttcaaggcccgggtggagacctcccggttcccccggtccaagaagcagcggatcctgctgcagaagttcgacgaggacggc |
| | | ttcaaggagtgcaacctgaacgacacccggtacgtgaaccgcttcctgtgccagttcgtggccgaccacatcctgctgaccgg |
| | | caagggcaagcggcgggtgttcgcctccaacggccagatcaccaacctgctgcggggcttctggggcctgcggaaggtgcggg |
| | | ccgagaacgaccggcaccacgccctggacgccgtggtggtggcctgctccaccgtggccatgcagcagaagatcacccggttc |
| | | gtgcggtacaaggagatgaacgccttcgacggcaagaccatcgacaaggagaccggcaaggtgctgcaccagaagacccactt |
| | | cccccagccctgggagttcttcgcccaggaggtgatgatccgggtgttcggcaagcccgacggcaagcccgagttcgaggagg |
| | | ccgacacccccgagaagctgcggaccctgctggccgagaagctgtcctcccggcccgaggccgtgcacgagtacgtgaccccc |
| | | ctgttcgtgtcccgggcccccaaccggaagatgtccggcgcccacaaggacaccctgcggtccgccaagcggttcgtgaagca |
| | | caacgagaagatctccgtgaagcgggtgtggctgaccgagatcaagctggccgacctggagaacatggtgaactacaagaacg |
| | | gccgggagatcgagctgtacgaggccctgaaggcccggctggaggcctacggcggcaacgccaagcaggccttcgaccccaag |
| | | gacaaccccttctacaagaagggcggccagctggtgaaggccgtgcgggtggagaagacccaggagtccggcgtgctgctgaa |
| | | caagaagaacgcctacaccatcgccgacaacggcgacatggtgcgggtggacgtgttctgcaaggtggacaagaagggcaaga |
| | | accagtacttcatcgtgcccatctacgcctggcaggtggccgagaacatcctgcccgacatcgactgcaagggctaccggatc |
| | | gacgactcctacaccttctgcttctccctgcacaagtacgacctgatcgccttccagaaggacgagaagtccaaggtggagtt |
| | | cgcctactacatcaactgcgactcctccaacggccggttctacctggcctggcacgacaagggctccaaggagcagcagttcc |
| | | ggatctccacccagaacctggtgctgatccagaagtaccaggtgaacgagctgggcaaggagatccggccctgccggctgaag |
| | | aagcggccccccgtgcggtag |
| ORF | 43 | ATGGACAAGAAGTACAGCATCGGACTGGACATCGGAACAAACAGCGTCGGATGGGCAGTCATCACAGACGAATACAAGGTCCC |
| encoding | | GAGCAAGAAGTTCAAGGTCCTGGGAAACACAGACAGACACAGCATCAAGAAGAACCTGATCGGAGCACTGCTGTTCGACAGCG |
| Sp. Cas9 | | GAGAAACAGCAGAAGCAACAAGACTGAAGAGAACAGCAAGAAGAAGATACACAAGAAGAAAGAACAGAATCTGCTACCTGCAG |
| | | GAAATCTTCAGCAACGAAATGGCAAAGGTCGACGACAGCTTCTTCCACAGACTGGAAGAAAGCTTCCTGGTCGAAGAAGACAA |
| | | GAAGCACGAAAGACACCCGATCTTCGGAAACATCGTCGACGAAGTCGCATACCACGAAAAGTACCCGACAATCTACCACCTGA |
| | | GAAAGAAGCTGGTCGACAGCACAGACAAGGCAGACCTGAGACTGATCTACCTGGCACTGGCACACATGATCAAGTTCAGAGGA |
| | | CACTTCCTGATCGAAGGAGACCTGAACCCGGACAACAGCGACGTCGACAAGCTGTTCATCCAGCTGGTCCAGACATACAACCA |
| | | GCTGTTCGAAGAAAACCCGATCAACGCAAGCGGAGTCGACGCAAAGGCAATCCTGAGCGCAAGACTGAGCAAGAGCAGAAGAC |
| | | TGGAAAACCTGATCGCACAGCTGCCGGGAGAAAAGAAGAACGGACTGTTCGGAAACCTGATCGCACTGAGCCTGGGACTGACA |
| | | CCGAACTTCAAGAGCAACTTCGACCTGGCAGAAGACGCAAAGCTGCAGCTGAGCAAGGACACATACGACGACGACCTGGACAA |
| | | CCTGCTGGCACAGATCGGAGACCAGTACGCAGACCTGTTCCTGGCAGCAAAGAACCTGAGCGACGCAATCCTGCTGAGCGACA |
| | | TCCTGAGAGTCAACACAGAAATCACAAAGGCACCGCTGAGCGCAAGCATGATCAAGAGATACGACGAACACCACCAGGACCTG |
| | | ACACTGCTGAAGGCACTGGTCAGACAGCAGCTGCCGGAAAAGTACAAGGAAATCTTCTTCGACCAGAGCAAGAACGGATACGC |
| | | AGGATACATCGACGGAGGAGCAAGCCAGGAAGAATTCTACAAGTTCATCAAGCCGATCCTGGAAAAGATGGACGGAACAGAAG |
| | | AACTGCTGGTCAAGCTGAACAGAGAAGACCTGCTGAGAAAGCAGAGAACATTCGACAACGGAAGCATCCCGCACCAGATCCAC |
| | | CTGGGAGAACTGCACGCAATCCTGAGAAGACAGGAAGACTTCTACCCGTTCCTGAAGGACAACAGAGAAAAGATCGAAAAGAT |
| | | CCTGACATTCAGAATCCCGTACTACGTCGGACCGCTGGCAAGAGGAAACAGCAGATTCGCATGGATGACAAGAAAGAGCGAAG |
| | | AAACAATCACACCGTGGAACTTCGAAGAAGTCGTCGACAAGGGAGCAAGCGCACAGAGCTTCATCGAAAGAATGACAAACTTC |
| | | GACAAGAACCTGCCGAACGAAAAGGTCCTGCCGAAGCACAGCCTGCTGTACGAATACTTCACAGTCTACAACGAACTGACAAA |
| | | GGTCAAGTACGTCACAGAAGGAATGAGAAAGCCGGCATTCCTGAGCGGAGAACAGAAGAAGGCAATCGTCGACCTGCTGTTCA |
| | | AGACAAACAGAAAGGTCACAGTCAAGCAGCTGAAGGAAGACTACTTCAAGAAGATCGAATGCTTCGACAGCGTCGAAATCAGC |
| | | GGAGTCGAAGACAGATTCAACGCAAGCCTGGGAACATACCACGACCTGCTGAAGATCATCAAGGACAAGGACTTCCTGGACAA |
| | | CGAAGAAAACGAAGACATCCTGGAAGACATCGTCCTGACACTGACACTGTTCGAAGACAGAGAAATGATCGAAGAAAGACTGA |
| | | AGACATACGCACACCTGTTCGACGACAAGGTCATGAAGCAGCTGAAGAGAAGAAGATACACAGGATGGGGAAGACTGAGCAGA |
| | | AAGCTGATCAACGGAATCAGAGACAAGCAGAGCGGAAAGACAATCCTGGACTTCCTGAAGAGCGACGGATTCGCAAACAGAAA |
| | | CTTCATGCAGCTGATCCACGACGACAGCCTGACATTCAAGGAAGACATCCAGAAGGCACAGGTCAGCGGACAGGGAGACAGCC |
| | | TGCACGAACACATCGCAAACCTGGCAGGAAGCCCGGCAATCAAGAAGGGAATCCTGCAGACAGTCAAGGTCGTCGACGAACTG |
| | | GTCAAGGTCATGGGAAGACACAAGCCGGAAAACATCGTCATCGAAATGGCAAGAGAAAACCAGACAACACAGAAGGGACAGAA |
| | | GAACAGCAGAGAAAGAATGAAGAGAATCGAAGAAGGAATCAAGGAACTGGGAAGCCAGATCCTGAAGGAACACCCGGTCGAAA |
| | | ACACACAGCTGCAGAACGAAAAGCTGTACCTGTACTACCTGCAGAACGGAAGAGACATGTACGTCGACCAGGAACTGGACATC |
| | | AACAGACTGAGCGACTACGACGTCGACCACATCGTCCCGCAGAGCTTCCTGAAGGACGACAGCATCGACAACAAGGTCCTGAC |
| | | AAGAAGCGACAAGAACAGAGGAAAGAGCGACAACGTCCCGAGCGAAGAAGTCGTCAAGAAGATGAAGAACTACTGGAGACAGC |
| | | TGCTGAACGCAAAGCTGATCACACAGAGAAAGTTCGACAACCTGACAAAGGCAGAGAGAGGAGGACTGAGCGAACTGGACAAG |
| | | GCAGGATTCATCAAGAGACAGCTGGTCGAAACAAGACAGATCACAAAGCACGTCGCACAGATCCTGGACAGCAGAATGAACAC |
| | | AAAGTACGACGAAAACGACAAGCTGATCAGAGAAGTCAAGGTCATCACACTGAAGAGCAAGCTGGTCAGCGACTTCAGAAAGG |
| | | ACTTCCAGTTCTACAAGGTCAGAGAAATCAACAACTACCACCACGCACACGACGCATACCTGAACGCAGTCGTCGGAACAGCA |
| | | CTGATCAAGAAGTACCCGAAGCTGGAAAGCGAATTCGTCTACGGAGACTACAAGGTCTACGACGTCAGAAAGATGATCGCAAA |
| | | GAGCGAACAGGAAATCGGAAAGGCAACAGCAAAGTACTTCTTCTACAGCAACATCATGAACTTCTTCAAGACAGAAATCACAC |
| | | TGGCAAACGGAGAAATCAGAAAGAGACCGCTGATCGAAACAAACGGAGAAACAGGAGAAATCGTCTGGGACAAGGGAAGAGAC |
| | | TTCGCAACAGTCAGAAAGGTCCTGAGCATGCCGCAGGTCAACATCGTCAAGAAGACAGAAGTCCAGACAGGAGGATTCAGCAA |
| | | GGAAAGCATCCTGCCGAAGAGAAACAGCGACAAGCTGATCGCAAGAAAGAAGGACTGGGACCCGAAGAAGTACGGAGGATTCG |
| | | ACAGCCCGACAGTCGCATACAGCGTCCTGGTCGTCGCAAAGGTCGAAAAGGGAAAGAGCAAGAAGCTGAAGAGCGTCAAGGAA |
| | | CTGCTGGGAATCACAATCATGGAAAGAAGCAGCTTCGAAAAGAACCCGATCGACTTCCTGGAAGCAAAGGGATACAAGGAAGT |
| | | CAAGAAGGACCTGATCATCAAGCTGCCGAAGTACAGCCTGTTCGAACTGGAAAACGGAAGAAAGAGAATGCTGGCAAGCGCAG |
| | | GAGAACTGCAGAAGGGAAACGAACTGGCACTGCCGAGCAAGTACGTCAACTTCCTGTACCTGGCAAGCCACTACGAAAAGCTG |
| | | AAGGGAAGCCCGGAAGACAACGAACAGAAGCAGCTGTTCGTCGAACAGCACAAGCACTACCTGGACGAAATCATCGAACAGAT |
| | | CAGCGAATTCAGCAAGAGAGTCATCCTGGCAGACGCAAACCTGGACAAGGTCCTGAGCGCATACAACAAGCACAGAGACAAGC |
| | | CGATCAGAGAACAGGCAGAAAACATCATCCACCTGTTCACACTGACAAACCTGGGAGCACCGGCAGCATTCAAGTACTTCGAC |
| | | ACAACAATCGACAGAAAGAGATACACAAGCACAAAGGAAGTCCTGGACGCAACACTGATCCACCAGAGCATCACAGGACTGTA |
| | | CGAAACAAGAATCGACCTGAGCCAGCTGGGAGGAGACGGAGGAGGAAGCCCGAAGAAGAAGAGAAAGGTCTAG |
| ORF | 44 | ATGGACAAGAAGTACTCCATCGGCCTGGACATCGGCACCAACTCCGTGGGCTGGGCCGTGATCACCGACGAGTACAAGGTGCC |
| encoding | | CTCCAAGAAGTTCAAGGTGCTGGGCAACACCGACCGGCACTCCATCAAGAAGAACCTGATCGGCGCCCTGCTGTTCGACTCCG |
| Sp. Cas9 | | GCGAGACCGCCGAGGCCACCCGGCTGAAGCGGACCGCCCGGCGGCGGTACACCCGGCGGAAGAACCGGATCTGCTACCTGCAG |
| | | GAGATCTTCTCCAACGAGATGGCCAAGGTGGACGACTCCTTCTTCCACCGGCTGGAGGAGTCCTTCCTGGTGGAGGAGGACAA |
| | | GAAGCACGAGCGGCACCCCATCTTCGGCAACATCGTGGACGAGGTGGCCTACCACGAGAAGTACCCCACCATCTACCACCTGC |
| | | GGAAGAAGCTGGTGGACTCCACCGACAAGGCCGACCTGCGGCTGATCTACCTGGCCCTGGCCCACATGATCAAGTTCCGGGGC |
| | | CACTTCCTGATCGAGGGCGACCTGAACCCCGACAACTCCGACGTGGACAAGCTGTTCATCCAGCTGGTGCAGACCTACAACCA |
| | | GCTGTTCGAGGAGAACCCCATCAACGCCTCCGGCGTGGACGCCAAGGCCATCCTGTCCGCCCGGCTGTCCAAGTCCCGGCGGC |
| | | TGGAGAACCTGATCGCCCAGCTGCCCGGCGAGAAGAAGAACGGCCTGTTCGGCAACCTGATCGCCCTGTCCCTGGGCCTGACC |
| | | CCCAACTTCAAGTCCAACTTCGACCTGGCCGAGGACGCCAAGCTGCAGCTGTCCAAGGACACCTACGACGACGACCTGGACAA |
| | | CCTGCTGGCCCAGATCGGCGACCAGTACGCCGACCTGTTCCTGGCCGCCAAGAACCTGTCCGACGCCATCCTGCTGTCCGACA |
| | | TCCTGCGGGTGAACACCGAGATCACCAAGGCCCCCCTGTCCGCCTCCATGATCAAGCGGTACGACGAGCACCACCAGGACCTG |
| | | ACCCTGCTGAAGGCCCTGGTGCGGCAGCAGCTGCCCGAGAAGTACAAGGAGATCTTCTTCGACCAGTCCAAGAACGGCTACGC |
| | | CGGCTACATCGACGGCGGCGCCTCCCAGGAGGAGTTCTACAAGTTCATCAAGCCCATCCTGGAGAAGATGGACGGCACCGAGG |
| | | AGCTGCTGGTGAAGCTGAACCGGGAGGACCTGCTGCGGAAGCAGCGGACCTTCGACAACGGCTCCATCCCCCACCAGATCCAC |
| | | CTGGGCGAGCTGCACGCCATCCTGCGGCGGCAGGAGGACTTCTACCCCTTCCTGAAGGACAACCGGGAGAAGATCGAGAAGAT |
| | | CCTGACCTTCCGGATCCCCTACTACGTGGGCCCCCTGGCCCGGGGCAACTCCCGGTTCGCCTGGATGACCCGGAAGTCCGAGG |
| | | AGACCATCACCCCCTGGAACTTCGAGGAGGTGGTGGACAAGGGCGCCTCCGCCCAGTCCTTCATCGAGCGGATGACCAACTTC |
| | | GACAAGAACCTGCCCAACGAGAAGGTGCTGCCCAAGCACTCCCTGCTGTACGAGTACTTCACCGTGTACAACGAGCTGACCAA |
| | | GGTGAAGTACGTGACCGAGGGCATGCGGAAGCCCGCCTTCCTGTCCGGCGAGCAGAAGAAGGCCATCGTGGACCTGCTGTTCA |
| | | AGACCAACCGGAAGGTGACCGTGAAGCAGCTGAAGGAGGACTACTTCAAGAAGATCGAGTGCTTCGACTCCGTGGAGATCTCC |
| | | GGCGTGGAGGACCGGTTCAACGCCTCCCTGGGCACCTACCACGACCTGCTGAAGATCATCAAGGACAAGGACTTCCTGGACAA |
| | | CGAGGAGAACGAGGACATCCTGGAGGACATCGTGCTGACCCTGACCCTGTTCGAGGACCGGGAGATGATCGAGGAGCGGCTGA |
| | | AGACCTACGCCCACCTGTTCGACGACAAGGTGATGAAGCAGCTGAAGCGGCGGCGGTACACCGGCTGGGGCCGGCTGTCCCGG |
| | | AAGCTGATCAACGGCATCCGGGACAAGCAGTCCGGCAAGACCATCCTGGACTTCCTGAAGTCCGACGGCTTCGCCAACCGGAA |
| | | CTTCATGCAGCTGATCCACGACGACTCCCTGACCTTCAAGGAGGACATCCAGAAGGCCCAGGTGTCCGGCCAGGGCGACTCCC |
| | | TGCACGAGCACATCGCCAACCTGGCCGGCTCCCCCGCCATCAAGAAGGGCATCCTGCAGACCGTGAAGGTGGTGGACGAGCTG |
| | | GTGAAGGTGATGGGCCGGCACAAGCCCGAGAACATCGTGATCGAGATGGCCCGGGAGAACCAGACCACCCAGAAGGGCCAGAA |
| | | GAACTCCCGGGAGCGGATGAAGCGGATCGAGGAGGGCATCAAGGAGCTGGGCTCCCAGATCCTGAAGGAGCACCCCGTGGAGA |
| | | ACACCCAGCTGCAGAACGAGAAGCTGTACCTGTACTACCTGCAGAACGGCCGGGACATGTACGTGGACCAGGAGCTGGACATC |
| | | AACCGGCTGTCCGACTACGACGTGGACCACATCGTGCCCCAGTCCTTCCTGAAGGACGACTCCATCGACAACAAGGTGCTGAC |
| | | CCGGTCCGACAAGAACCGGGGCAAGTCCGACAACGTGCCCTCCGAGGAGGTGGTGAAGAAGATGAAGAACTACTGGCGGCAGC |
| | | TGCTGAACGCCAAGCTGATCACCCAGCGGAAGTTCGACAACCTGACCAAGGCCGAGCGGGGCGGCCTGTCCGAGCTGGACAAG |
| | | GCCGGCTTCATCAAGCGGCAGCTGGTGGAGACCCGGCAGATCACCAAGCACGTGGCCCAGATCCTGGACTCCCGGATGAACAC |
| | | CAAGTACGACGAGAACGACAAGCTGATCCGGGAGGTGAAGGTGATCACCCTGAAGTCCAAGCTGGTGTCCGACTTCCGGAAGG |
| | | ACTTCCAGTTCTACAAGGTGCGGGAGATCAACAACTACCACCACGCCCACGACGCCTACCTGAACGCCGTGGTGGGCACCGCC |
| | | CTGATCAAGAAGTACCCCAAGCTGGAGTCCGAGTTCGTGTACGGCGACTACAAGGTGTACGACGTGCGGAAGATGATCGCCAA |
| | | GTCCGAGCAGGAGATCGGCAAGGCCACCGCCAAGTACTTCTTCTACTCCAACATCATGAACTTCTTCAAGACCGAGATCACCC |
| | | TGGCCAACGGCGAGATCCGGAAGCGGCCCCTGATCGAGACCAACGGCGAGACCGGCGAGATCGTGTGGGACAAGGGCCGGGAC |
| | | TTCGCCACCGTGCGGAAGGTGCTGTCCATGCCCCAGGTGAACATCGTGAAGAAGACCGAGGTGCAGACCGGCGGCTTCTCCAA |
| | | GGAGTCCATCCTGCCCAAGCGGAACTCCGACAAGCTGATCGCCCGGAAGAAGGACTGGGACCCCAAGAAGTACGGCGGCTTCG |
| | | ACTCCCCCACCGTGGCCTACTCCGTGCTGGTGGTGGCCAAGGTGGAGAAGGGCAAGTCCAAGAAGCTGAAGTCCGTGAAGGAG |
| | | CTGCTGGGCATCACCATCATGGAGCGGTCCTCCTTCGAGAAGAACCCCATCGACTTCCTGGAGGCCAAGGGCTACAAGGAGGT |
| | | GAAGAAGGACCTGATCATCAAGCTGCCCAAGTACTCCCTGTTCGAGCTGGAGAACGGCCGGAAGCGGATGCTGGCCTCCGCCG |
| | | GCGAGCTGCAGAAGGGCAACGAGCTGGCCCTGCCCTCCAAGTACGTGAACTTCCTGTACCTGGCCTCCCACTACGAGAAGCTG |
| | | AAGGGCTCCCCCGAGGACAACGAGCAGAAGCAGCTGTTCGTGGAGCAGCACAAGCACTACCTGGACGAGATCATCGAGCAGAT |
| | | CTCCGAGTTCTCCAAGCGGGTGATCCTGGCCGACGCCAACCTGGACAAGGTGCTGTCCGCCTACAACAAGCACCGGGACAAGC |
| | | CCATCCGGGAGCAGGCCGAGAACATCATCCACCTGTTCACCCTGACCAACCTGGGCGCCCCCGCCGCCTTCAAGTACTTCGAC |
| | | ACCACCATCGACCGGAAGCGGTACACCTCCACCAAGGAGGTGCTGGACGCCACCCTGATCCACCAGTCCATCACCGGCCTGTA |
| | | CGAGACCCGGATCGACCTGTCCCAGCTGGGCGGCGACGGCGGCGGCTCCCCCAAGAAGAAGCGGAAGGTGTGA |
| ORF | 45 | AUGGACAAGAAGUACAGCAUCGGCCUGGACAUCGGCACGAACAGCGUUGGCUGGGCUGUGAUCACGGACGAGUACAAGGUUCC |
| encoding | | CUCAAAGAAGUUCAAGGUGCUGGGCAACACGGACCGGCACAGCAUCAAGAAGAAUCUCAUCGGUGCACUGCUGUUCGACAGCG |
| Sp. Cas9 | | GUGAGACGGCCGAAGCCACGCGGCUGAAGCGGACGGCCCGCCGGCGGUACACGCGGCGGAAGAACCGGAUCUGCUACCUGCAG |
| | | GAGAUCUUCAGCAACGAGAUGGCCAAGGUGGACGACAGCUUCUUCCACCGGCUGGAGGAGAGCUUCCUGGUGGAGGAGGACAA |
| | | GAAGCACGAGCGGCACCCCAUCUUCGGCAACAUCGUGGACGAAGUCGCCUACCACGAGAAGUACCCCACCAUCUACCACCUGC |
| | | GGAAGAAGCUGGUGGACUCGACUGACAAGGCCGACCUGCGGCUGAUCUACCUGGCACUGGCCCACAUGAUAAAGUUCCGGGGC |
| | | CACUUCCUGAUCGAGGGCGACCUGAACCCUGACAACAGCGACGUGGACAAGCUGUUCAUCCAGCUGGUGCAGACCUACAACCA |
| | | GCUGUUCGAGGAGAACCCCAUCAACGCCAGCGGCGUGGACGCCAAGGCCAUCCUCAGCGCCCGCCUCAGCAAGAGCCGGCGGC |
| | | UGGAGAAUCUCAUCGCCCAGCUUCCAGGUGAGAAGAAGAAUGGGCUGUUCGGCAAUCUCAUCGCACUCAGCCUGGGCCUGACU |
| | | CCCAACUUCAAGAGCAACUUCGACCUGGCCGAGGACGCCAAGCUGCAGCUCAGCAAGGACACCUACGACGACGACCUGGACAA |
| | | UCUCCUGGCCCAGAUCGGCGACCAGUACGCCGACCUGUUCCUGGCUGCCAAGAAUCUCAGCGACGCCAUCCUGCUCAGCGACA |
| | | UCCUGCGGGUGAACACAGAGAUCACGAAGGCCCCCCUCAGCGCCAGCAUGAUAAAGCGGUACGACGAGCACCACCAGGACCUG |
| | | ACGCUGCUGAAGGCACUGGUGCGGCAGCAGCUUCCAGAGAAGUACAAGGAGAUCUUCUUCGACCAGAGCAAGAAUGGGUACGC |
| | | CGGGUACAUCGACGGUGGUGCCAGCCAGGAGGAGUUCUACAAGUUCAUCAAGCCCAUCCUGGAGAAGAUGGACGGCACAGAGG |
| | | AGCUGCUGGUGAAGCUGAACAGGGAGGACCUGCUGCGGAAGCAGCGGACGUUCGACAAUGGGAGCAUCCCCCACCAGAUCCAC |
| | | CUGGGUGAGCUGCACGCCAUCCUGCGGCGGCAGGAGGACUUCUACCCCUUCCUGAAGGACAACAGGGAGAAGAUCGAGAAGAU |
| | | CCUGACGUUCCGGAUCCCCUACUACGUUGGCCCCCUGGCCCGCGGCAACAGCCGGUUCGCCUGGAUGACGCGGAAGAGCGAGG |
| | | AGACGAUCACUCCCUGGAACUUCGAGGAAGUCGUGGACAAGGGUGCCAGCGCCCAGAGCUUCAUCGAGCGGAUGACGAACUUC |
| | | GACAAGAAUCUUCCAAACGAGAAGGUGCUUCCAAAGCACAGCCUGCUGUACGAGUACUUCACGGUGUACAACGAGCUGACGAA |
| | | GGUGAAGUACGUGACAGAGGGCAUGCGGAAGCCCGCCUUCCUCAGCGGUGAGCAGAAGAAGGCCAUCGUGGACCUGCUGUUCA |
| | | AGACGAACCGGAAGGUGACGGUGAAGCAGCUGAAGGAGGACUACUUCAAGAAGAUCGAGUGCUUCGACAGCGUGGAGAUCAGC |
| | | GGCGUGGAGGACCGGUUCAACGCCAGCCUGGGCACCUACCACGACCUGCUGAAGAUCAUCAAGGACAAGGACUUCCUGGACAA |
| | | CGAGGAGAACGAGGACAUCCUGGAGGACAUCGUGCUGACGCUGACGCUGUUCGAGGACAGGGAGAUGAUAGAGGAGCGGCUGA |
| | | AGACCUACGCCCACCUGUUCGACGACAAGGUGAUGAAGCAGCUGAAGCGGCGGCGGUACACGGGCUGGGGCCGGCUCAGCCGG |
| | | AAGCUGAUCAAUGGGAUCCGAGACAAGCAGAGCGGCAAGACGAUCCUGGACUUCCUGAAGAGCGACGGCUUCGCCAACCGGAA |
| | | CUUCAUGCAGCUGAUCCACGACGACAGCCUGACGUUCAAGGAGGACAUCCAGAAGGCCCAGGUCAGCGGCCAGGGCGACAGCC |
| | | UGCACGAGCACAUCGCCAAUCUCGCCGGGAGCCCCGCCAUCAAGAAGGGGAUCCUGCAGACGGUGAAGGUGGUGGACGAGCUG |
| | | GUGAAGGUGAUGGGCCGGCACAAGCCAGAGAACAUCGUGAUCGAGAUGGCCAGGGAGAACCAGACGACUCAAAAGGGGCAGAA |
| | | GAACAGCAGGGAGCGGAUGAAGCGGAUCGAGGAGGGCAUCAAGGAGCUGGGCAGCCAGAUCCUGAAGGAGCACCCCGUGGAGA |
| | | ACACUCAACUGCAGAACGAGAAGCUGUACCUGUACUACCUGCAGAAUGGGCGAGACAUGUACGUGGACCAGGAGCUGGACAUC |
| | | AACCGGCUCAGCGACUACGACGUGGACCACAUCGUUCCCCAGAGCUUCCUGAAGGACGACAGCAUCGACAACAAGGUGCUGAC |
| | | GCGGAGCGACAAGAACCGGGGCAAGAGCGACAACGUUCCCUCAGAGGAAGUCGUGAAGAAGAUGAAGAACUACUGGCGGCAGC |
| | | UGCUGAACGCCAAGCUGAUCACUCAACGGAAGUUCGACAAUCUCACGAAGGCCGAGCGGGGUGGCCUCAGCGAGCUGGACAAG |
| | | GCCGGGUUCAUCAAGCGGCAGCUGGUGGAGACGCGGCAGAUCACGAAGCACGUGGCCCAGAUCCUGGACAGCCGGAUGAACAC |
| | | GAAGUACGACGAGAACGACAAGCUGAUCAGGGAAGUCAAGGUGAUCACGCUGAAGAGCAAGCUGGUCAGCGACUUCCGGAAGG |
| | | ACUUCCAGUUCUACAAGGUGAGGGAGAUCAACAACUACCACCACGCCCACGACGCCUACCUGAACGCUGUGGUUGGCACGGCA |
| | | CUGAUCAAGAAGUACCCCAAGCUGGAGAGCGAGUUCGUGUACGGCGACUACAAGGUGUACGACGUGCGGAAGAUGAUAGCCAA |
| | | GAGCGAGCAGGAGAUCGGCAAGGCCACGGCCAAGUACUUCUUCUACAGCAACAUCAUGAACUUCUUCAAGACAGAGAUCACGC |
| | | UGGCCAAUGGUGAGAUCCGGAAGCGGCCCCUGAUCGAGACGAAUGGUGAGACGGGUGAGAUCGUGUGGGACAAGGGGCGAGAC |
| | | UUCGCCACGGUGCGGAAGGUGCUCAGCAUGCCCCAGGUGAACAUCGUGAAGAAGACAGAAGUCCAGACGGGUGGCUUCAGCAA |
| | | GGAGAGCAUCCUUCCAAAGCGGAACAGCGACAAGCUGAUCGCCCGCAAGAAGGACUGGGACCCCAAGAAGUACGGUGGCUUCG |
| | | ACAGCCCCACCGUGGCCUACAGCGUGCUGGUGGUGGCCAAGGUGGAGAAGGGGAAGAGCAAGAAGCUGAAGAGCGUGAAGGAG |
| | | CUGCUGGGCAUCACGAUCAUGGAGCGGAGCAGCUUCGAGAAGAACCCCAUCGACUUCCUGGAAGCCAAGGGGUACAAGGAAGU |
| | | CAAGAAGGACCUGAUCAUCAAGCUUCCAAAGUACAGCCUGUUCGAGCUGGAGAAUGGGCGGAAGCGGAUGCUGGCCAGCGCCG |
| | | GUGAGCUGCAGAAGGGGAACGAGCUGGCACUUCCCUCAAAGUACGUGAACUUCCUGUACCUGGCCAGCCACUACGAGAAGCUG |
| | | AAGGGGAGCCCAGAGGACAACGAGCAGAAGCAGCUGUUCGUGGAGCAGCACAAGCACUACCUGGACGAGAUCAUCGAGCAGAU |
| | | CAGCGAGUUCAGCAAGCGGGUGAUCCUGGCCGACGCCAAUCUCGACAAGGUGCUCAGCGCCUACAACAAGCACCGAGACAAGC |
| | | CCAUCAGGGAGCAGGCCGAGAACAUCAUCCACCUGUUCACGCUGACGAAUCUCGGUGCCCCCGCUGCCUUCAAGUACUUCGAC |
| | | ACGACGAUCGACCGGAAGCGGUACACGUCGACUAAGGAAGUCCUGGACGCCACGCUGAUCCACCAGAGCAUCACGGGCCUGUA |
| | | CGAGACGCGGAUCGACCUCAGCCAGCUGGGUGGCGACGGUGGUGGCAGCCCCAAGAAGAAGCGGAAGGUGUAG |
| ORF | 46 | AUGGACAAGAAGUACAGCAUCGGCCUCGACAUCGGCACCAACAGCGUCGGCUGGGCCGUCAUCACCGACGAGUACAAGGUCCC |
| encoding | | CAGCAAGAAGUUCAAGGUCCUCGGCAACACCGACCGCCACAGCAUCAAGAAGAACCUCAUCGGCGCCCUCCUCUUCGACAGCG |
| Sp. Cas9 | | GCGAGACCGCCGAGGCCACCCGCCUCAAGCGCACCGCCCGCCGCCGCUACACCCGCCGCAAGAACCGCAUCUGCUACCUCCAG |
| | | GAGAUCUUCAGCAACGAGAUGGCCAAGGUCGACGACAGCUUCUUCCACCGCCUCGAGGAGAGCUUCCUCGUCGAGGAGGACAA |
| | | GAAGCACGAGCGCCACCCCAUCUUCGGCAACAUCGUCGACGAGGUCGCCUACCACGAGAAGUACCCCACCAUCUACCACCUCC |
| | | GCAAGAAGCUCGUCGACAGCACCGACAAGGCCGACCUCCGCCUCAUCUACCUCGCCCUCGCCCACAUGAUCAAGUUCCGCGGC |
| | | CACUUCCUCAUCGAGGGCGACCUCAACCCCGACAACAGCGACGUCGACAAGCUCUUCAUCCAGCUCGUCCAGACCUACAACCA |
| | | GCUCUUCGAGGAGAACCCCAUCAACGCCAGCGGCGUCGACGCCAAGGCCAUCCUCAGCGCCCGCCUCAGCAAGAGCCGCCGCC |
| | | UCGAGAACCUCAUCGCCCAGCUCCCCGGCGAGAAGAAGAACGGCCUCUUCGGCAACCUCAUCGCCCUCAGCCUCGGCCUCACC |
| | | CCCAACUUCAAGAGCAACUUCGACCUCGCCGAGGACGCCAAGCUCCAGCUCAGCAAGGACACCUACGACGACGACCUCGACAA |
| | | CCUCCUCGCCCAGAUCGGCGACCAGUACGCCGACCUCUUCCUCGCCGCCAAGAACCUCAGCGACGCCAUCCUCCUCAGCGACA |
| | | UCCUCCGCGUCAACACCGAGAUCACCAAGGCCCCCCUCAGCGCCAGCAUGAUCAAGCGCUACGACGAGCACCACCAGGACCUC |
| | | ACCCUCCUCAAGGCCCUCGUCCGCCAGCAGCUCCCCGAGAAGUACAAGGAGAUCUUCUUCGACCAGAGCAAGAACGGCUACGC |
| | | CGGCUACAUCGACGGCGGCGCCAGCCAGGAGGAGUUCUACAAGUUCAUCAAGCCCAUCCUCGAGAAGAUGGACGGCACCGAGG |
| | | AGCUCCUCGUCAAGCUCAACCGCGAGGACCUCCUCCGCAAGCAGCGCACCUUCGACAACGGCAGCAUCCCCCACCAGAUCCAC |
| | | CUCGGCGAGCUCCACGCCAUCCUCCGCCGCCAGGAGGACUUCUACCCCUUCCUCAAGGACAACCGCGAGAAGAUCGAGAAGAU |
| | | CCUCACCUUCCGCAUCCCCUACUACGUCGGCCCCCUCGCCCGCGGCAACAGCCGCUUCGCCUGGAUGACCCGCAAGAGCGAGG |
| | | AGACCAUCACCCCCUGGAACUUCGAGGAGGUCGUCGACAAGGGCGCCAGCGCCCAGAGCUUCAUCGAGCGCAUGACCAACUUC |
| | | GACAAGAACCUCCCCAACGAGAAGGUCCUCCCCAAGCACAGCCUCCUCUACGAGUACUUCACCGUCUACAACGAGCUCACCAA |
| | | GGUCAAGUACGUCACCGAGGGCAUGCGCAAGCCCGCCUUCCUCAGCGGCGAGCAGAAGAAGGCCAUCGUCGACCUCCUCUUCA |
| | | AGACCAACCGCAAGGUCACCGUCAAGCAGCUCAAGGAGGACUACUUCAAGAAGAUCGAGUGCUUCGACAGCGUCGAGAUCAGC |
| | | GGCGUCGAGGACCGCUUCAACGCCAGCCUCGGCACCUACCACGACCUCCUCAAGAUCAUCAAGGACAAGGACUUCCUCGACAA |
| | | CGAGGAGAACGAGGACAUCCUCGAGGACAUCGUCCUCACCCUCACCCUCUUCGAGGACCGCGAGAUGAUCGAGGAGCGCCUCA |
| | | AGACCUACGCCCACCUCUUCGACGACAAGGUCAUGAAGCAGCUCAAGCGCCGCCGCUACACCGGCUGGGGCCGCCUCAGCCGC |
| | | AAGCUCAUCAACGGCAUCCGCGACAAGCAGAGCGGCAAGACCAUCCUCGACUUCCUCAAGAGCGACGGCUUCGCCAACCGCAA |
| | | CUUCAUGCAGCUCAUCCACGACGACAGCCUCACCUUCAAGGAGGACAUCCAGAAGGCCCAGGUCAGCGGCCAGGGCGACAGCC |
| | | UCCACGAGCACAUCGCCAACCUCGCCGGCAGCCCCGCCAUCAAGAAGGGCAUCCUCCAGACCGUCAAGGUCGUCGACGAGCUC |
| | | GUCAAGGUCAUGGGCCGCCACAAGCCCGAGAACAUCGUCAUCGAGAUGGCCCGCGAGAACCAGACCACCCAGAAGGGCCAGAA |
| | | GAACAGCCGCGAGCGCAUGAAGCGCAUCGAGGAGGGCAUCAAGGAGCUCGGCAGCCAGAUCCUCAAGGAGCACCCCGUCGAGA |
| | | ACACCCAGCUCCAGAACGAGAAGCUCUACCUCUACUACCUCCAGAACGGCCGCGACAUGUACGUCGACCAGGAGCUCGACAUC |
| | | AACCGCCUCAGCGACUACGACGUCGACCACAUCGUCCCCCAGAGCUUCCUCAAGGACGACAGCAUCGACAACAAGGUCCUCAC |
| | | CCGCAGCGACAAGAACCGCGGCAAGAGCGACAACGUCCCCAGCGAGGAGGUCGUCAAGAAGAUGAAGAACUACUGGCGCCAGC |
| | | UCCUCAACGCCAAGCUCAUCACCCAGCGCAAGUUCGACAACCUCACCAAGGCCGAGCGCGGCGGCCUCAGCGAGCUCGACAAG |
| | | GCCGGCUUCAUCAAGCGCCAGCUCGUCGAGACCCGCCAGAUCACCAAGCACGUCGCCCAGAUCCUCGACAGCCGCAUGAACAC |
| | | CAAGUACGACGAGAACGACAAGCUCAUCCGCGAGGUCAAGGUCAUCACCCUCAAGAGCAAGCUCGUCAGCGACUUCCGCAAGG |
| | | ACUUCCAGUUCUACAAGGUCCGCGAGAUCAACAACUACCACCACGCCCACGACGCCUACCUCAACGCCGUCGUCGGCACCGCC |
| | | CUCAUCAAGAAGUACCCCAAGCUCGAGAGCGAGUUCGUCUACGGCGACUACAAGGUCUACGACGUCCGCAAGAUGAUCGCCAA |
| | | GAGCGAGCAGGAGAUCGGCAAGGCCACCGCCAAGUACUUCUUCUACAGCAACAUCAUGAACUUCUUCAAGACCGAGAUCACCC |
| | | UCGCCAACGGCGAGAUCCGCAAGCGCCCCCUCAUCGAGACCAACGGCGAGACCGGCGAGAUCGUCUGGGACAAGGGCCGCGAC |
| | | UUCGCCACCGUCCGCAAGGUCCUCAGCAUGCCCCAGGUCAACAUCGUCAAGAAGACCGAGGUCCAGACCGGCGGCUUCAGCAA |
| | | GGAGAGCAUCCUCCCCAAGCGCAACAGCGACAAGCUCAUCGCCCGCAAGAAGGACUGGGACCCCAAGAAGUACGGCGGCUUCG |
| | | ACAGCCCCACCGUCGCCUACAGCGUCCUCGUCGUCGCCAAGGUCGAGAAGGGCAAGAGCAAGAAGCUCAAGAGCGUCAAGGAG |
| | | CUCCUCGGCAUCACCAUCAUGGAGCGCAGCAGCUUCGAGAAGAACCCCAUCGACUUCCUCGAGGCCAAGGGCUACAAGGAGGU |
| | | CAAGAAGGACCUCAUCAUCAAGCUCCCCAAGUACAGCCUCUUCGAGCUCGAGAACGGCCGCAAGCGCAUGCUCGCCAGCGCCG |
| | | GCGAGCUCCAGAAGGGCAACGAGCUCGCCCUCCCCAGCAAGUACGUCAACUUCCUCUACCUCGCCAGCCACUACGAGAAGCUC |
| | | AAGGGCAGCCCCGAGGACAACGAGCAGAAGCAGCUCUUCGUCGAGCAGCACAAGCACUACCUCGACGAGAUCAUCGAGCAGAU |
| | | CAGCGAGUUCAGCAAGCGCGUCAUCCUCGCCGACGCCAACCUCGACAAGGUCCUCAGCGCCUACAACAAGCACCGCGACAAGC |
| | | CCAUCCGCGAGCAGGCCGAGAACAUCAUCCACCUCUUCACCCUCACCAACCUCGGCGCCCCCGCCGCCUUCAAGUACUUCGAC |
| | | ACCACCAUCGACCGCAAGCGCUACACCAGCACCAAGGAGGUCCUCGACGCCACCCUCAUCCACCAGAGCAUCACCGGCCUCUA |
| | | CGAGACCCGCAUCGACCUCAGCCAGCUCGGCGGCGACGGCGGCGGCAGCCCCAAGAAGAAGCGCAAGGUCUAG |
| ORF | 47 | ATGGACAAGAAGTACAGCATCGGCCTGGACATCGGCACCAACAGCGTGGGCTGGGCCGTGATCACCGACGAGTACAAGGTGCC |
| encoding | | CAGCAAGAAGTTCAAGGTGCTGGGCAACACCGACCGGCACAGCATCAAGAAGAACCTGATCGGCGCCCTGCTGTTCGACAGCG |
| Sp. Cas9 | | GCGAGACCGCCGAGGCCACCCGGCTGAAGCGGACCGCCCGGCGGCGGTACACCCGGCGGAAGAACCGGATCTGCTACCTGCAG |
| | | GAGATCTTCAGCAACGAGATGGCCAAGGTGGACGACAGCTTCTTCCACCGGCTGGAGGAGAGCTTCCTGGTGGAGGAGGACAA |
| | | GAAGCACGAGCGGCACCCCATCTTCGGCAACATCGTGGACGAGGTGGCCTACCACGAGAAGTACCCCACCATCTACCACCTGC |
| | | GGAAGAAGCTGGTGGACAGCACCGACAAGGCCGACCTGCGGCTGATCTACCTGGCCCTGGCCCACATGATCAAGTTCCGGGGC |
| | | CACTTCCTGATCGAGGGCGACCTGAACCCCGACAACAGCGACGTGGACAAGCTGTTCATCCAGCTGGTGCAGACCTACAACCA |
| | | GCTGTTCGAGGAGAACCCCATCAACGCCAGCGGCGTGGACGCCAAGGCCATCCTGAGCGCCCGGCTGAGCAAGAGCCGGCGGC |
| | | TGGAGAACCTGATCGCCCAGCTGCCCGGCGAGAAGAAGAACGGCCTGTTCGGCAACCTGATCGCCCTGAGCCTGGGCCTGACC |
| | | CCCAACTTCAAGAGCAACTTCGACCTGGCCGAGGACGCCAAGCTGCAGCTGAGCAAGGACACCTACGACGACGACCTGGACAA |
| | | CCTGCTGGCCCAGATCGGCGACCAGTACGCCGACCTGTTCCTGGCCGCCAAGAACCTGAGCGACGCCATCCTGCTGAGCGACA |
| | | TCCTGCGGGTGAACACCGAGATCACCAAGGCCCCCCTGAGCGCCAGCATGATCAAGCGGTACGACGAGCACCACCAGGACCTG |
| | | ACCCTGCTGAAGGCCCTGGTGCGGCAGCAGCTGCCCGAGAAGTACAAGGAGATCTTCTTCGACCAGAGCAAGAACGGCTACGC |
| | | CGGCTACATCGACGGCGGCGCCAGCCAGGAGGAGTTCTACAAGTTCATCAAGCCCATCCTGGAGAAGATGGACGGCACCGAGG |
| | | AGCTGCTGGTGAAGCTGAACCGGGAGGACCTGCTGCGGAAGCAGCGGACCTTCGACAACGGCAGCATCCCCCACCAGATCCAC |
| | | CTGGGCGAGCTGCACGCCATCCTGCGGCGGCAGGAGGACTTCTACCCCTTCCTGAAGGACAACCGGGAGAAGATCGAGAAGAT |
| | | CCTGACCTTCCGGATCCCCTACTACGTGGGCCCCCTGGCCCGGGGCAACAGCCGGTTCGCCTGGATGACCCGGAAGAGCGAGG |
| | | AGACCATCACCCCCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAACGAGCTGACCAA |
| | | GGTGAAGTACGTGACCGAGGGCATGCGGAAGCCCGCCTTCCTGAGCGGCGAGCAGAAGAAGGCCATCGTGGACCTGCTGTTCA |
| | | AGACCAACCGGAAGGTGACCGTGAAGCAGCTGAAGGAGGACTACTTCAAGAAGATCGAGTGCTTCGACAGCGTGGAGATCAGC |
| | | GGCGTGGAGGACCGGTTCAACGCCAGCCTGGGCACCTACCACGACCTGCTGAAGATCATCAAGGACAAGGACTTCCTGGACAA |
| | | CGAGGAGAACGAGGACATCCTGGAGGACATCGTGCTGACCCTGACCCTGTTCGAGGACCGGGAGATGATCGAGGAGCGGCTGA |
| | | AGACCTACGCCCACCTGTTCGACGACAAGGTGATGAAGCAGCTGAAGCGGCGGCGGTACACCGGCTGGGGCCGGCTGAGCCGG |
| | | AAGCTGATCAACGGCATCCGGGACAAGCAGAGCGGCAAGACCATCCTGGACTTCCTGAAGAGCGACGGCTTCGCCAACCGGAA |
| | | CTTCATGCAGCTGATCCACGACGACAGCCTGACCTTCAAGGAGGACATCCAGAAGGCCCAGGTGAGCGGCCAGGGCGACAGCC |
| | | TGCACGAGCACATCGCCAACCTGGCCGGCAGCCCCGCCATCAAGAAGGGCATCCTGCAGACCGTGAAGGTGGTGGACGAGCTG |
| | | GTGAAGGTGATGGGCCGGCACAAGCCCGAGAACATCGTGATCGAGATGGCCCGGGAGAACCAGACCACCCAGAAGGGCCAGAA |
| | | GAACAGCCGGGAGCGGATGAAGCGGATCGAGGAGGGCATCAAGGAGCTGGGCAGCCAGATCCTGAAGGAGCACCCCGTGGAGA |
| | | ACACCCAGCTGCAGAACGAGAAGCTGTACCTGTACTACCTGCAGAACGGCCGGGACATGTACGTGGACCAGGAGCTGGACATC |
| | | AACCGGCTGAGCGACTACGACGTGGACCACATCGTGCCCCAGAGCTTCCTGAAGGACGACAGCATCGACAACAAGGTGCTGAC |
| | | CCGGAGCGACAAGAACCGGGGCAAGAGCGACAACGTGCCCAGCGAGGAGGTGGTGAAGAAGATGAAGAACTACTGGCGGCAGC |
| | | TGCTGAACGCCAAGCTGATCACCCAGCGGAAGTTCGACAACCTGACCAAGGCCGAGCGGGGCGGCCTGAGCGAGCTGGACAAG |
| | | GCCGGCTTCATCAAGCGGCAGCTGGTGGAGACCCGGCAGATCACCAAGCACGTGGCCCAGATCCTGGACAGCCGGATGAACAC |
| | | CAAGTACGACGAGAACGACAAGCTGATCCGGGAGGTGAAGGTGATCACCCTGAAGAGCAAGCTGGTGAGCGACTTCCGGAAGG |
| | | ACTTCCAGTTCTACAAGGTGCGGGAGATCAACAACTACCACCACGCCCACGACGCCTACCTGAACGCCGTGGTGGGCACCGCC |
| | | CTGATCAAGAAGTACCCCAAGCTGGAGAGCGAGTTCGTGTACGGCGACTACAAGGTGTACGACGTGCGGAAGATGATCGCCAA |
| | | GAGCGAGCAGGAGATCGGCAAGGCCACCGCCAAGTACTTCTTCTACAGCAACATCATGAACTTCTTCAAGACCGAGATCACCC |
| | | TGGCCAACGGCGAGATCCGGAAGCGGCCCCTGATCGAGACCAACGGCGAGACCGGCGAGATCGTGTGGGACAAGGGCCGGGAC |
| | | TTCGCCACCGTGCGGAAGGTGCTGAGCATGCCCCAGGTGAACATCGTGAAGAAGACCGAGGTGCAGACCGGCGGCTTCAGCAA |
| | | GGAGAGCATCCTGCCCAAGCGGAACAGCGACAAGCTGATCGCCCGGAAGAAGGACTGGGACCCCAAGAAGTACGGCGGCTTCG |
| | | ACAGCCCCACCGTGGCCTACAGCGTGCTGGTGGTGGCCAAGGTGGAGAAGGGCAAGAGCAAGAAGCTGAAGAGCGTGAAGGAG |
| | | CTGCTGGGCATCACCATCATGGAGCGGAGCAGCTTCGAGAAGAACCCCATCGACTTCCTGGAGGCCAAGGGCTACAAGGAGGT |
| | | GAAGAAGGACCTGATCATCAAGCTGCCCAAGTACAGCCTGTTCGAGCTGGAGAACGGCCGGAAGCGGATGCTGGCCAGCGCCG |
| | | GCGAGCTGCAGAAGGGCAACGAGCTGGCCCTGCCCAGCAAGTACGTGAACTTCCTGTACCTGGCCAGCCACTACGAGAAGCTG |
| | | AAGGGCAGCCCCGAGGACAACGAGCAGAAGCAGCTGTTCGTGGAGCAGCACAAGCACTACCTGGACGAGATCATCGAGCAGAT |
| | | CAGCGAGTTCAGCAAGCGGGTGATCCTGGCCGACGCCAACCTGGACAAGGTGCTGAGCGCCTACAACAAGCACCGGGACAAGC |
| | | CCATCCGGGAGCAGGCCGAGAACATCATCCACCTGTTCACCCTGACCAACCTGGGCGCCCCCGCCGCCTTCAAGTACTTCGAC |
| | | ACCACCATCGACCGGAAGCGGTACACCAGCACCAAGGAGGTGCTGGACGCCACCCTGATCCACCAGAGCATCACCGGCCTGTA |
| | | CGAGACCCGGATCGACCTGAGCCAGCTGGGCGGCGACGGCGGCGGCAGCCCCAAGAAGAAGCGGAAGGTGTGA |
| amino | 48 | MDKKYSIGLDIGTNSVGWAVITDEYKVPSKKFKVLGNTDRHSIKKNLIGALLEDSGETAEATRLKRTARRRYTRRKNRICYLQ |
| acid | | EIFSNEMAKVDDSFFHRLEESELVEEDKKHERHPIFGNIVDEVAYHEKYPTIYHLRKKLVDSTDKADLRLIYLALAHMIKERG |
| sequence | | HFLIEGDLNPDNSDVDKLFIQLVQTYNQLFEENPINASGVDAKAILSARLSKSRRLENLIAQLPGEKKNGLFGNLIALSLGLT |
| for Sp. | | PNFKSNEDLAEDAKLQLSKDTYDDDLDNLLAQIGDQYADLELAAKNLSDAILLSDILRVNTEITKAPLSASMIKRYDEHHQDL |
| Cas9 | | TLLKALVRQQLPEKYKEIFFDQSKNGYAGYIDGGASQEEFYKFIKPILEKMDGTEELLVKLNREDLLRKQRTEDNGSIPHQIH |
| | | LGELHAILRRQEDFYPELKDNREKIEKILTFRIPYYVGPLARGNSRFAWMTRKSEETITPWNFEEVVDKGASAQSFIERMTNE |
| | | DKNLPNEKVLPKHSLLYEYFTVYNELTKVKYVTEGMRKPAFLSGEQKKAIVDLLFKTNRKVTVKQLKEDYFKKIECFDSVEIS |
| | | GVEDRENASLGTYHDLLKIIKDKDELDNEENEDILEDIVLTLTLFEDREMIEERLKTYAHLEDDKVMKQLKRRRYTGWGRLSR |
| | | KLINGIRDKQSGKTILDFLKSDGFANRNEMQLIHDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQILKEHPVENTQLQNEKLYLYYLQNGRDMYVDQELDI |
| | | NRLSDYDVDHIVPQSFLKDDSIDNKVLTRSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDERKDFQFYKVREINNYHHAHDAYLNAVVGTA |
| | | LIKKYPKLESEFVYGDYKVYDVRKMIAKSEQEIGKATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKRMLASAGELQKGNELALPSKYVNFLYLASHYEKL |
| | | KGSPEDNEQKQLFVEQHKHYLDEIIEQISEFSKRVILADANLDKVLSAYNKHRDKPIREQAENIIHLETLTNLGAPAAFKYED |
| | | TTIDRKRYTSTKEVLDATLIHQSITGLYETRIDLSQLGGDGGGSPKKKRKV |
| Open | 49 | AUGGACAAGAAGUACUCCAUCGGCCUGGACAUCGGCACCAACUCCGUGGGCUGGGCCGUGAUCACCGACGAGUACAAGGUGCC |
| reading | | CUCCAAGAAGUUCAAGGUGCUGGGCAACACCGACCGGCACUCCAUCAAGAAGAACCUGAUCGGCGCCCUGCUGUUCGACUCCG |
| frame for | | GCGAGACCGCCGAGGCCACCCGGCUGAAGCGGACCGCCCGGCGGCGGUACACCCGGCGGAAGAACCGGAUCUGCUACCUGCAG |
| Cas9 with | | GAGAUCUUCUCCAACGAGAUGGCCAAGGUGGACGACUCCUUCUUCCACCGGCUGGAGGAGUCCUUCCUGGUGGAGGAGGACAA |
| HiBiT tag | | GAAGCACGAGCGGCACCCCAUCUUCGGCAACAUCGUGGACGAGGUGGCCUACCACGAGAAGUACCCCACCAUCUACCACCUGC |
| | | GGAAGAAGCUGGUGGACUCCACCGACAAGGCCGACCUGCGGCUGAUCUACCUGGCCCUGGCCCACAUGAUCAAGUUCCGGGGC |
| | | CACUUCCUGAUCGAGGGCGACCUGAACCCCGACAACUCCGACGUGGACAAGCUGUUCAUCCAGCUGGUGCAGACCUACAACCA |
| | | GCUGUUCGAGGAGAACCCCAUCAACGCCUCCGGCGUGGACGCCAAGGCCAUCCUGUCCGCCCGGCUGUCCAAGUCCCGGCGGC |
| | | UGGAGAACCUGAUCGCCCAGCUGCCCGGCGAGAAGAAGAACGGCCUGUUCGGCAACCUGAUCGCCCUGUCCCUGGGCCUGACC |
| | | CCCAACUUCAAGUCCAACUUCGACCUGGCCGAGGACGCCAAGCUGCAGCUGUCCAAGGACACCUACGACGACGACCUGGACAA |
| | | CCUGCUGGCCCAGAUCGGCGACCAGUACGCCGACCUGUUCCUGGCCGCCAAGAACCUGUCCGACGCCAUCCUGCUGUCCGACA |
| | | UCCUGCGGGUGAACACCGAGAUCACCAAGGCCCCCCUGUCCGCCUCCAUGAUCAAGCGGUACGACGAGCACCACCAGGACCUG |
| | | ACCCUGCUGAAGGCCCUGGUGCGGCAGCAGCUGCCCGAGAAGUACAAGGAGAUCUUCUUCGACCAGUCCAAGAACGGCUACGC |
| | | CGGCUACAUCGACGGCGGCGCCUCCCAGGAGGAGUUCUACAAGUUCAUCAAGCCCAUCCUGGAGAAGAUGGACGGCACCGAGG |
| | | AGCUGCUGGUGAAGCUGAACCGGGAGGACCUGCUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUCCUGAAGGACAACCGGGAGAAGAUCGAGAAGAU |
| | | CCUGACCUUCCGGAUCCCCUACUACGUGGGCCCCCUGGCCCGGGGCAACUCCCGGUUCGCCUGGAUGACCCGGAAGUCCGAGG |
| | | AGACCAUCACCCCCUGGAACUUCGAGGAGGUGGUGGACAAGGGCGCCUCCGCCCAGUCCUUCAUCGAGCGGAUGACCAACUUC |
| | | GACAAGAACCUGCCCAACGAGAAGGUGCUGCCCAAGCACUCCCUGCUGUACGAGUACUUCACCGUGUACAACGAGCUGACCAA |
| | | GGUGAAGUACGUGACCGAGGGCAUGCGGAAGCCCGCCUUCCUGUCCGGCGAGCAGAAGAAGGCCAUCGUGGACCUGCUGUUCA |
| | | AGACCAACCGGAAGGUGACCGUGAAGCAGCUGAAGGAGGACUACUUCAAGAAGAUCGAGUGCUUCGACUCCGUGGAGAUCUCC |
| | | GGCGUGGAGGACCGGUUCAACGCCUCCCUGGGCACCUACCACGACCUGCUGAAGAUCAUCAAGGACAAGGACUUCCUGGACAA |
| | | CGAGGAGAACGAGGACAUCCUGGAGGACAUCGUGCUGACCCUGACCCUGUUCGAGGACCGGGAGAUGAUCGAGGAGCGGCUGA |
| | | AGACCUACGCCCACCUGUUCGACGACAAGGUGAUGAAGCAGCUGAAGCGGCGGCGGUACACCGGCUGGGGCCGGCUGUCCCGG |
| | | AAGCUGAUCAACGGCAUCCGGGACAAGCAGUCCGGCAAGACCAUCCUGGACUUCCUGAAGUCCGACGGCUUCGCCAACCGGAA |
| | | CUUCAUGCAGCUGAUCCACGACGACUCCCUGACCUUCAAGGAGGACAUCCAGAAGGCCCAGGUGUCCGGCCAGGGCGACUCCC |
| | | UGCACGAGCACAUCGCCAACCUGGCCGGCUCCCCCGCCAUCAAGAAGGGCAUCCUGCAGACCGUGAAGGUGGUGGACGAGCUG |
| | | GUGAAGGUGAUGGGCCGGCACAAGCCCGAGAACAUCGUGAUCGAGAUGGCCCGGGAGAACCAGACCACCCAGAAGGGCCAGAA |
| | | GAACUCCCGGGAGCGGAUGAAGCGGAUCGAGGAGGGCAUCAAGGAGCUGGGCUCCCAGAUCCUGAAGGAGCACCCCGUGGAGA |
| | | ACACCCAGCUGCAGAACGAGAAGCUGUACCUGUACUACCUGCAGAACGGCCGGGACAUGUACGUGGACCAGGAGCUGGACAUC |
| | | AACCGGCUGUCCGACUACGACGUGGACCACAUCGUGCCCCAGUCCUUCCUGAAGGACGACUCCAUCGACAACAAGGUGCUGAC |
| | | CCGGUCCGACAAGAACCGGGGCAAGUCCGACAACGUGCCCUCCGAGGAGGUGGUGAAGAAGAUGAAGAACUACUGGCGGCAGC |
| | | UGCUGAACGCCAAGCUGAUCACCCAGCGGAAGUUCGACAACCUGACCAAGGCCGAGCGGGGCGGCCUGUCCGAGCUGGACAAG |
| | | GCCGGCUUCAUCAAGCGGCAGCUGGUGGAGACCCGGCAGAUCACCAAGCACGUGGCCCAGAUCCUGGACUCCCGGAUGAACAC |
| | | CAAGUACGACGAGAACGACAAGCUGAUCCGGGAGGUGAAGGUGAUCACCCUGAAGUCCAAGCUGGUGUCCGACUUCCGGAAGG |
| | | ACUUCCAGUUCUACAAGGUGCGGGAGAUCAACAACUACCACCACGCCCACGACGCCUACCUGAACGCCGUGGUGGGCACCGCC |
| | | CUGAUCAAGAAGUACCCCAAGCUGGAGUCCGAGUUCGUGUACGGCGACUACAAGGUGUACGACGUGCGGAAGAUGAUCGCCAA |
| | | GUCCGAGCAGGAGAUCGGCAAGGCCACCGCCAAGUACUUCUUCUACUCCAACAUCAUGAACUUCUUCAAGACCGAGAUCACCC |
| | | UGGCCAACGGCGAGAUCCGGAAGCGGCCCCUGAUCGAGACCAACGGCGAGACCGGCGAGAUCGUGUGGGACAAGGGCCGGGAC |
| | | UUCGCCACCGUGCGGAAGGUGCUGUCCAUGCCCCAGGUGAACAUCGUGAAGAAGACCGAGGUGCAGACCGGCGGCUUCUCCAA |
| | | GGAGUCCAUCCUGCCCAAGCGGAACUCCGACAAGCUGAUCGCCCGGAAGAAGGACUGGGACCCCAAGAAGUACGGCGGCUUCG |
| | | ACUCCCCCACCGUGGCCUACUCCGUGCUGGUGGUGGCCAAGGUGGAGAAGGGCAAGUCCAAGAAGCUGAAGUCCGUGAAGGAG |
| | | CUGCUGGGCAUCACCAUCAUGGAGCGGUCCUCCUUCGAGAAGAACCCCAUCGACUUCCUGGAGGCCAAGGGCUACAAGGAGGU |
| | | GAAGAAGGACCUGAUCAUCAAGCUGCCCAAGUACUCCCUGUUCGAGCUGGAGAACGGCCGGAAGCGGAUGCUGGCCUCCGCCG |
| | | GCGAGCUGCAGAAGGGCAACGAGCUGGCCCUGCCCUCCAAGUACGUGAACUUCCUGUACCUGGCCUCCCACUACGAGAAGCUG |
| | | AAGGGCUCCCCCGAGGACAACGAGCAGAAGCAGCUGUUCGUGGAGCAGCACAAGCACUACCUGGACGAGAUCAUCGAGCAGAU |
| | | CUCCGAGUUCUCCAAGCGGGUGAUCCUGGCCGACGCCAACCUGGACAAGGUGCUGUCCGCCUACAACAAGCACCGGGACAAGC |
| | | CCAUCCGGGAGCAGGCCGAGAACAUCAUCCACCUGUUCACCCUGACCAACCUGGGCGCCCCCGCCGCCUUCAAGUACUUCGAC |
| | | ACCACCAUCGACCGGAAGCGGUACACCUCCACCAAGGAGGUGCUGGACGCCACCCUGAUCCACCAGUCCAUCACCGGCCUGUA |
| | | CGAGACCCGGAUCGACCUGUCCCAGCUGGGCGGCGACGGCGGCGGCUCCCCCAAGAAGAAGCGGAAGGUGUCCGAGUCCGCCA |
| | | CCCCCGAGUCCGUGUCCGGCUGGCGGCUGUUCAAGAAGAUCUCCUGA |
| Amino | 50 | MDKKYSIGLDIGTNSVGWAVITDEYKVPSKKFKVLGNTDRHSIKKNLIGALLFDSGETAEATRLKRTARRRYTRRKNRICYLQ |
| acid | | EIFSNEMAKVDDSFFHRLEESELVEEDKKHERHPIFGNIVDEVAYHEKYPTIYHLRKKLVDSTDKADLRLIYLALAHMIKERG |
| sequence | | HFLIEGDLNPDNSDVDKLFIQLVQTYNQLFEENPINASGVDAKAILSARLSKSRRLENLIAQLPGEKKNGLFGNLIALSLGLT |
| for Cas9 | | PNFKSNFDLAEDAKLQLSKDTYDDDLDNLLAQIGDQYADLFLAAKNLSDAILLSDILRVNTEITKAPLSASMIKRYDEHHQDL |
| with | | TLLKALVRQQLPEKYKEIFFDQSKNGYAGYIDGGASQEEFYKFIKPILEKMDGTEELLVKLNREDLLRKQRTEDNGSIPHQIH |
| HiBiT tag | | LGELHAILRRQEDFYPFLKDNREKIEKILTFRIPYYVGPLARGNSRFAWMTRKSEETITPWNFEEVVDKGASAQSFIERMTNF |
| | | DKNLPNEKVLPKHSLLYEYFTVYNELTKVKYVTEGMRKPAFLSGEQKKAIVDLLFKTNRKVTVKQLKEDYFKKIECFDSVEIS |
| | | GVEDRFNASLGTYHDLLKIIKDKDELDNEENEDILEDIVLTLTLFEDREMIEERLKTYAHLEDDKVMKQLKRRRYTGWGRLSR |
| | | KLINGIRDKQSGKTILDFLKSDGFANRNFMQLIHDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQILKEHPVENTQLQNEKLYLYYLQNGRDMYVDQELDI |
| | | NRLSDYDVDHIVPQSFLKDDSIDNKVLTRSDKNRGKSDNVPSEEVVKKMKNYWRQLLNAKLITQRKEDNLTKAERGGLSELDK |
| | | AGFIKRQLVETRQITKHVAQILDSRMNTKYDENDKLIREVKVITLKSKLVSDERKDFQFYKVREINNYHHAHDAYLNAVVGTA |
| | | LIKKYPKLESEFVYGDYKVYDVRKMIAKSEQEIGKATAKYFFYSNIMNFFKTEITLANGEIRKRPLIETNGETGEIVWDKGRD |
| | | FATVRKVLSMPQVNIVKKTEVQTGGESKESILPKRNSDKLIARKKDWDPKKYGGEDSPTVAYSVLVVAKVEKGKSKKLKSVKE |
| | | LLGITIMERSSFEKNPIDFLEAKGYKEVKKDLIIKLPKYSLFELENGRKRMLASAGELQKGNELALPSKYVNFLYLASHYEKL |
| | | KGSPEDNEQKQLFVEQHKHYLDEIIEQISEFSKRVILADANLDKVLSAYNKHRDKPIREQAENIIHLFTLTNLGAPAAFKYED |
| | | TTIDRKRYTSTKEVLDATLIHQSITGLYETRIDLSQLGGDGGGSPKKKRKVSESATPESVSGWRLEKKIS |
| Amino | 151 | MEASPASGPRHLMDPHIFTSNENNGIGRHKTYLCYEVERLDNGTSVKMDQHRGFLHNQAKNLLCGFYGRHAELRELDLVPSLQ |
| acid | | LDPAQIYRVTWFISWSPCFSWGCAGEVRAFLQENTHVRLRIFAARIYDYDPLYKEALQMLRDAGAQVSIMTYDEFKHCWDTEV |
| sequence | | DHQGCPFQPWDGLDEHSQALSGRLRAILQNQGN |
| of H. | | |
| sapiens | | |
| APOBEC3A | | |
| deaminase | | |
| (A3A) | | |
| Amino | 152 | MSSETGPVAVDPTLRRRIEPHEFEVFFDPRELRKETCLLYEINWGGRHSIWRHTSQNTNKHVEVNFIEKFTTERYFCPNTRCS |
| acid | | ITWFLSWSPCGECSRAITEFLSRYPHVTLFIYIARLYHHADPRNRQGLRDLISSGVTIQIMTEQESGYCWRNFVNYSPSNEAH |
| sequence | | WPRYPHLWVRLYVLELYCIILGLPPCLNILRRKQPQLTFFTIALQSCHYQRLPPHILWATGLK |
| of R. | | |
| norvegicus | | |
| Apobec1 | | |
| Exemplary | 153 | MSSETGPVAVDPTLRRRIEPHEFEVFEDPRELRKETCLLYEINWGGRHSVWRHTSQNTSNHVEVNFLEKFTTERYFRPNTRCS |
| amino | | ITWFLSWSPCGECSRAITEFLSRHPYVTLF |
| acid | | |
| sequences | | |
| for | | |
| cytidine | | |
| deaminase | | |
| Exemplary | 154 | MSSETGPVAVDPTLRRRIEPHEFEVFFDPRELRKETCLLYEINWGGRHSVWRHTSQNTSNHVEVNFLEKFTTERYFRPNTRCS |
| amino | | ITWFLSWSPCGECSRAITEFLSRHPYVTLFIYIARLYH |
| acid | | |
| sequences | | |
| for | | |
| cytidine | | |
| deaminase | | |
| Exemplary | 155 | MNWNALRSKAIEVSRHAYAPYSGFPVGAAALVDDGRTVTGCNVENVSYGLGLCAECAVVCALHSGGGGRLVALSCVGPDGGVL |
| amino | | MPCGRCRQVLLEHGGPELLIDHAHGPRPLRELLPDAFGPDDLGRR |
| acid | | |
| sequences | | |
| for | | |
| cytidine | | |
| deaminase | | |
| Exemplary | 156 | MTHHALIEAAKAAREKAYAPYSNEKVGAALVTNDGKVFHGCNVENASYGLCNCAERTALESALAAGYRPGEFAAIAVVGETHG |
| amino | | PIAPCGACRQVMIELGKPTLEVVLTNMQGDVRVTSAGDLLPDAFYLA |
| acid | | |
| sequences | | |
| for | | |
| cytidine | | |
| deaminase | | |
| Exemplary | 157 | MPDIDWKQLRDKATQVAAGAYAPYSRFPVGAAALVDDGRVVTGCNVENVSYGLALCAECGVVCALHATGGGRLVALACVDGRG |
| amino | | APLMPCGRCRQLLFEHGGPELLVDHLAGPRRLGDLLPEPFHADLTGEPT |
| acid | | |
| sequences | | |
| for | | |
| cytidine | | |
| deaminase | | |
| Exemplary | 158 | MNSKQLIQEAIEARKQAYVPYSKFQVGAALLTQDGKVYRGCNVENASYGLCNCAERTALFKAVSEGDKEFVAIAIVADTKRPV |
| amino | | PPCGACRQVMVELCKQDTKVYLSNLHGDVQETTVGELLPGAFLAEDLHE |
| acid | | |
| sequences | | |
| for | | |
| cytidine | | |
| deaminase | | |
| Exemplary | 159 | MPDVDWNMLRGNATQAAAGAYVPYSRFAVGAAALVDDGRVVTGCNVENVSYGLTLCAECAVVCALHSTGGGRLLALACVDGHG |
| amino | | SVLMPCGRCRQVLLEHGGSELLIDHPVRPRRLGDLLPDAFGLDDLPRERR |
| acid | | |
| sequences | | |
| for | | |
| cytidine | | |
| deaminase | | |
| Exemplary | 160 | MGDVNWDTLQKAAVAARANSYAPYSNFPVGVAGFVNDGRLITGVNVENASYGLALCAECSMISALYATGGGRLVAVYCVDGNG |
| amino | | DSLMPCGRCRQLLYEHGGPELKIMTPKGVQTMAQLLPQAFNPQERIFGNDE |
| acid | | |
| sequences | | |
| for | | |
| cytidine | | |
| deaminase | | |
| Exemplary | 161 | MNRQELITEALKARDMAYAPYSKFQVGAALLTKDGKVYRGCNIENAAYSMCNCAERTALFKAVSEGDTEFQMLAVAADTPGPV |
| amino | | SPCGACRQVISELCTKDVIVVLTNLQGQIKEMTVEELLPGAFSSEDLHDERKL |
| acid | | |
| sequences | | |
| for | | |
| cytidine | | |
| deaminase | | |
| Exemplary | 162 | MKVGGIEDRQLEALKRAALKACELSYSPYSHFRVGCSILTNNDVIFTGANVENASYSNCICAERSAMIQVLMAGHRSGWKCMV |
| amino | | ICGDSEDQCVSPCGVCRQFINEFVVKDFPIVMLNSTGSRSKVMTMGELLPMAFGPSHLN |
| acid | | |
| sequences | | |
| for | | |
| cytidine | | |
| deaminase | | |
| Exemplary | 163 | MNIEQQLYDVVKQLIEQRYPNDWGGAAAIRVEDGTIYTSVAPDVINASTELCMETGAILEAHKFQKKVTHSICLARENEHSEL |
| amino | | KVLSPCGVCQERLFYWGPEVQCAITNAKQDIIFKPLKELQPYHWTEAYHDEMVKEWSTR |
| acid | | |
| sequences | | |
| for | | |
| cytidine | | |
| deaminase | | |
| Exemplary | 164 | MAQERPSCAVEPEHVQRLLLSSREAKKSAYCPYSRFPVGAALLTGDGRIFSGCNIENACYPLGVCAERTAIQKAISEGYKDER |
| amino | | AIAISSDLQEEFISPCGACRQVMREFGTDWAVYMTKPDGTFVVRTVQELLPASFGPEDLQKIQ |
| acid | | |
| sequences | | |
| for | | |
| cytidine | | |
| deaminase | | |
| Exemplary | 165 | MAQKRPACTLKPECVQQLLVCSQEAKKSAYCPYSHFPVGAALLTQEGRIFKGCNIENACYPLGICAERTAIQKAVSEGYKDER |
| amino | | AIAIASDMQDDFISPCGACRQVMREFGTNWPVYMTKPDGTYIVMTVQELLPSSFGPEDLQKTQ |
| acid | | |
| sequences | | |
| for | | |
| cytidine | | |
| deaminase | | |
| Exemplary | 166 | MVTGGMASKWDQKGMDIAYEEAALGYKEGGVPIGGCLINNKDGSVLGRGHNMRFQKGSATLHGEISTLENCGRLEGKVYKDTT |
| amino | | LYTTLSPCDMCTGAIIMYGIPRCVVGENVNFKSKGEKYLQTRGHEVVVVDDERCKKIMKQFIDERPQDWFEDIGE |
| acid | | |
| sequences | | |
| for | | |
| cytidine | | |
| deaminase | | |
| Exemplary | 167 | MTTTKANLTEFEQQLVDKAIGAMENAYCKYSNFKVGAALVCDDGEIIIGANHENASYGATICAERSAIVTALTKGHRKFKYIV |
| amino | | VATELEAPCSPCGVCRQVLIEFGDYKVILGSSTSDQIIETTTYELLPYAFTPKSLDDHEKETEERKHHNDHNNKE |
| acid | | |
| sequences | | |
| for | | |
| cytidine | | |
| deaminase | | |
| Exemplary | 168 | MAANSLPQDISDVELVHLARAAMKRAHCPYSKFPVGAALLTESGEIVQGCNVENASYGGTICAERSAIVSAVSQGYTKFRAIA |
| amino | | VVTELSEPASPCGLCRQFLVEFGDYKVVVGTASNNKILITSTRALLPFAFTPESLDTFEQEKASEAKGLKQDDATEHNVTVVS |
| acid | | |
| sequences | | |
| for | | |
| cytidine | | |
| deaminase | | |
| Exemplary | 169 | MNPQIRNPMKAMYPGTFYFQFKNLWEANDRNETWLCFTVEGIKRRSVVSWKTGVERNQVDSETHCHAERCFLSWFCDDILSPN |
| amino | | TKYQVTWYTSWSPCPDCAGEVAEFLARHSNVNLTIFTARLYYFQYPCYQEGLRSLSQEGVAVEIMDYEDEKYCWENEVYNDNE |
| acid | | PFKPWKGLKTNFRLLKRRLRESLQ |
| sequences | | |
| for | | |
| cytidine | | |
| deaminase | | |
| Exemplary | 170 | MNPQIRNPMEAMYPHIFYFHFKNLLKACGRNESWLCFTMEVTKHHSAVFRKRGVFRNQVDPETHCHAERCFLSWFCDDILSPN |
| amino | | TNYEVTWYTSWSPCPECAGEVAEFLARHSNVNLTIFTARLCYFWDTDYQEGLCSLSQEGASVKIMGYKDFVSCWKNEVYSDDE |
| acid | | PFKPWKGLQTNFRLLKRRLREILQ |
| sequences | | |
| for | | |
| cytidine | | |
| deaminase | | |
| Exemplary | 171 | MNDALHIGLPPFLVQANNEPRVLAAPEARMGYVLELVRANIAADGGPFAAAVFERDSGLLIAAGTNRVVPGRCSAAHAEILAL |
| amino | | SLAQAKLDTHDLSADGLPACELVTSAEPCVMCFGAVIWSGVRSLVCAARSDDVEAIGEDEGPRPENWMGGLEARGITVTTGLL |
| acid | | RDAACALLREYNACNGVIYNARCGVHK |
| sequences | | |
| for | | |
| cytidine | | |
| deaminase | | |
| Exemplary | 172 | MDSLLMNRRKFLYQFKNVRWAKGRRETYLCYVVKRRDSATSFSLDEGYLRNKNGCHVELLFLRYISDWDLDPGRCYRVTWETS |
| amino | | WSPCYDCARHVADFLRGNPNLSLRIFTARLYFCEDRKAEPEGLRRLHRAGVQIAIMTFKDYFYCWNTEVENHERTFKAWEGLH |
| acid | | ENSVRLSRQLRRILLPLYEVDDLRDAFRTLGL |
| sequences | | |
| for | | |
| cytidine | | |
| deaminase | | |
| Exemplary | 173 | MDSLLMKQKKFLYHFKNVRWAKGRHETYLCYVVKRRDSATSCSLDFGHLRNKSGCHVELLFLRYISDWDLDPGRCYRVTWFTS |
| amino | | WSPCYDCARHVAEFLRWNPNLSLRIFTARLYFCEDRKAEPEGLRRLHRAGVQIGIMTFKDYFYCWNTEVENRERTFKAWEGLH |
| acid | | ENSVRLTRQLRRILLPLYEVDDLRDAFRMLGF |
| sequences | | |
| for | | |
| cytidine | | |
| deaminase | | |
| Exemplary | 174 | MALLTAETFRLQFNNKRRLRRPYYPRKALLCYQLTPQNGSTPTRGYFENKKKCHAEICFINEIKSMGLDETQCYQVTCYLTWS |
| amino | | PCSSCAWELVDFIKAHDHLNLGIFASRLYYHWCKPQQKGLRLLCGSQVPVEVMGFPKFADCWENFVDHEKPLSFNPYKMLEEL |
| acid | | DKNSRAIKRRLERIKIPGVRAQGRYMDILCDAEV |
| sequences | | |
| for | | |
| cytidine | | |
| deaminase | | |
| Exemplary | 175 | MAQKEEAAVATEAASQNGEDLENLDDPEKLKELIELPPFEIVTGERLPANFFKFQFRNVEYSSGRNKTFLCYVVEAQGKGGQV |
| amino | | QASRGYLEDEHAAAHAEEAFENTILPAFDPALRYNVTWYVSSSPCAACADRIIKTLSKTKNLRLLILVGRLFMWEEPEIQAAL |
| acid | | KKLKEAGCKLRIMKPQDFEYVWQNFVEQEEGESKAFQPWEDIQENFLYYEEKLADILK |
| sequences | | |
| for | | |
| cytidine | | |
| deaminase | | |
| Exemplary | 176 | MAQKEEAAEAAAPASQNGDDLENLEDPEKLKELIDLPPFEIVTGVRLPVNFFKFQFRNVEYSSGRNKTFLCYVVEVQSKGGQA |
| amino | | QATQGYLEDEHAGAHAEEAFFNTILPAFDPALKYNVTWYVSSSPCAACADRILKTLSKTKNLRLLILVSRLEMWEEPEVQAAL |
| acid | | KKLKEAGCKLRIMKPQDFEYIWQNFVEQEEGESKAFEPWEDIQENFLYYEEKLADILK |
| sequences | | |
| for | | |
| cytidine | | |
| deaminase | | |
| Exemplary | 177 | MSSETGPVAVDPTLRRRIEPHEFEVFFDPRELRKETCLLYEINWGGRHSVWRHTSQNTSNHVEVNFLEKFTTERYFRPNTRCS |
| amino | | ITWFLSWSPCGECSRAITEFLSRHPYVTLFIYIARLYHHTDQRNRQGLRDLISSGVTIQIMTEQEYCYCWRNFVNYPPSNEAY |
| acid | | WPRYPHLWVKLYVLELYCIILGLPPCLKILRRKQPQLTFFTITLQTCHYQRIPPHLLWATGLK |
| sequences | | |
| for | | |
| cytidine | | |
| deaminase | | |
| Exemplary | 178 | MNSKTGPSVGDATLRRRIKPWEFVAFFNPQELRKETCLLYEIKWGNQNIWRHSNQNTSQHAEINFMEKFTAERHENSSVRCSI |
| amino | | TWFLSWSPCWECSKAIRKFLDHYPNVTLAIFISRLYWHMDQQHRQGLKELVHSGVTIQIMSYSEYHYCWRNFVDYPQGEEDYW |
| acid | | PKYPYLWIMLYVLELHCIILGLPPCLKISGSHSNQLALESLDLQDCHYQKIPYNVLVATGLVQPFVTWR |
| sequences | | |
| for | | |
| cytidine | | |
| deaminase | | |
| Exemplary | 179 | MTSEKGPSTGDPTLRRRIEPWEFDVFYDPRELRKEACLLYEIKWGMSRKIWRSSGKNTTNHVEVNFIKKFTSERDFHPSMSCS |
| amino | | ITWFLSWSPCWECSQAIREFLSRHPGVTLVIYVARLFWHMDQQNRQGLRDLVNSGVTIQIMRASEYYHCWRNFVNYPPGDEAH |
| acid | | WPQYPPLWMMLYALELHCIILSLPPCLKISRRWQNHLTFFRLHLQNCHYQTIPPHILLATGLIHPSVAWR |
| sequences | | |
| for | | |
| cytidine | | |
| deaminase | | |
| Exemplary | 180 | MASEKGPSNKDYTLRRRIEPWEFEVEFDPQELRKEACLLYEIKWGASSKTWRSSGKNTINHVEVNFLEKLTSEGRLGPSTCCS |
| amino | | ITWFLSWSPCWECSMAIREFLSQHPGVTLIIFVARLFQHMDRRNRQGLKDLVTSGVTVRVMSVSEYCYCWENFVNYPPGKAAQ |
| acid | | WPRYPPRWMLMYALELYCIILGLPPCLKISRRHQKQLTFFSLTPQYCHYKMIPPYILLATGLLQPSVPWR |
| sequences | | |
| for | | |
| cytidine | | |
| deaminase | | |
| Exemplary | 181 | MKVSLAGQTVDVKKILNEIPKRTVTAALLEGGEIVAVEEADDEHAERKLVRRHDVEGKVVFVTARPCLYCARELAEAGVAGVV |
| amino | | YLGRGRGLGPYYLARSGVEVVEVHPDEPLGYDPVDRLDVLLTFGGNPYLTEEDVAARVYCLLTGRGFDADIAPAPENLSGRVE |
| acid | | IMVTRGDPDEAVELLKEELPVERIRRFLISGEFDRDELRERILEDIEPRILDPFAVRARIARAGAFSSSREAEVFIGDVLTSV |
| sequences | | GREVNLNDPRTVVTVDVLGPRVSVGVEKR |
| for | | |
| cytidine | | |
| deaminase | | |
| Exemplary | 182 | MHPRFQTAFAQLADNLQSALEPILADKYFPALLTGEQVSSLKSATGLDEDALAFALLPLAAACARTPLSNENVGAIARGVSGT |
| amino | | WYFGANMEFIGATMQQTVHAEQSAISHAWLSGEKALAAITVNYTPCGHCRQEMNELNSGLDLRIHLPGREAHALRDYLPDAFG |
| acid | | PKDLEIKTLLMDEQDHGYALTGDALSQAAIAAANRSHMPYSKSPSGVALECKDGRIFSGSYAENAAFNPTLPPLQGALILLNL |
| sequences | | KGYDYPDIQRAVLAEKADAPLIQWDATSATLKALGCHSIDRVLLA |
| for | | |
| cytidine | | |
| deaminase | | |
| Exemplary | 183 | MRNRIEQALQQMPASFAPYLRELVLAKDEDATESAEQYQQLLTLSGLEDADLRVALLPIAAAYSYAPISEFYVGAIVRGISGR |
| amino | | LYLGANMEFTGAQLGQTVHAEQCAISHAWMKGEKGVADITINFSPCGHCRQFMNELTTASSLKIQLPKRAAKTLQEYLPESFG |
| acid | | PADLGIDSGLMSPVNHGKTSDDDEELIQQALRAMNISHSPYTQNFSGVALKMRSGAIYLGAYAENAAFNPSLPPLQVALAQAM |
| sequences | | MMGESFEDIEAAALVESATGKISHLADTQATLEVINPDIPLSYLSL |
| for | | |
| cytidine | | |
| deaminase | | |
| Exemplary | 184 | MSWSPCFECAEQIVRFLATHHNLSLDIFSSRLYNVQDPETQQNLCRLVQEGAQVAAMDLYEFKKCWKKFVDNGGRRFRPWKRL |
| amino | | LTNFRYQDSKLQEILRRMDPLSEEEFYSQFYNQRVKHLCYYHRMKPYLCYQLEQFNGQAPLKGCLLSEKGKQHAEILFLDKIR |
| acid | | SMELSQVTITCYLTWSPCPNCAWQLAAFKRDRPDLILHIYTSRLYFHWKRPFQKGLCSLWQSGILVDVMDLPQFTDCWTNFVN |
| sequences | | PKRPFWPWKGLEIISRRTQRRLRRIKESWGLQDLVNDFGNLQLGPPMS |
| for | | |
| cytidine | | |
| deaminase | | |
| Exemplary | 185 | MDKPSFVIQSKEAESAAKQLGVSVIQLLPSLVKPAQSYARTPISKFNVAVVGLGSSGRIFLGVNVEFPNLPLHHSIHAEQFLV |
| amino | | TNLTLNGERHLNFFAVSAAPCGHCRQFLQEIRDAPEIKILITDPNNSADSDSAADSDGFLRLGSFLPHRFGPDDLLGKDHPLL |
| acid | | LESHDNHLKISDLDSICNGNTDSSADLKQTALAAANRSYAPYSLCPSGVSLVDCDGKVYRGWYMESAAYNPSMGPVQAALVDY |
| sequences | | VANGGGGGYERIVGAVLVEKEDAVVRQEHTARLLLETISPKCEFKVFHCYEA |
| for | | |
| cytidine | | |
| deaminase | | |
| Exemplary | 186 | MVEPMDPRTFVSNENNRPILSGLNTVWLCCEVKTKDPSGPPLDAKIFQGKVYSKAKYHPEMRFLRWFHKWRQLHHDQEYKVTW |
| amino | | YVSWSPCTRCANSVATFLAKDPKVTLTIFVARLYYFWKPDYQQALRILCQKRGGPHATMKIMNYNEFQDCWNKFVDGRGKPFK |
| acid | | PRNNLPKHYTLLQATLGELLRHLMDPGTFTSNFNNKPWVSGQHETYLCYKVERLHNDTWVPLNQHRGFLRNQAPNIHGFPKGR |
| sequences | | HAELCFLDLIPFWKLDGQQYRVTCFTSWSPCFSCAQEMAKFISNNEHVSLCIFAARIYDDQGRYQEGLRALHRDGAKIAMMNY |
| for | | SEFEYCWDTFVDRQGRPFQPWDGLDEHSQALSGRLRAI |
| cytidine | | |
| deaminase | | |
| Exemplary | 187 | MKPHFRNTVERMYRDTFSYNFYNRPILSRRNTVWLCYEVKTKGPSRPRLDAKIFRGQVYSQPEHHAEMCFLSWFCGNQLPAYK |
| amino | | CFQITWFVSWTPCPDCVAKLAEFLAEHPNVTLTISAARLYYYWERDYRRALCRLSQAGARVKIMDDEEFAYCWENFVYSEGQP |
| acid | | FMPWYKFDDNYAFLHRTLKEILRNPMEAMYPHIFYFHFKNLRKAYGRNESWLCFTMEVVKHHSPVSWKRGVERNQVDPETHCH |
| sequences | | AERCFLSWFCDDILSPNTNYEVTWYTSWSPCPECAGEVAEFLARHSNVNLTIFTARLYYFWDTDYQEGLRSLSQEGASVEIMG |
| for | | YKDFKYCWENFVYNDDEPFKPWKGLKYNFLFLDSKLQEILE |
| cytidine | | |
| deaminase | | |
| Exemplary | 188 | MNPQIRNMVEQMEPDIFVYYFNNRPILSGRNTVWLCYEVKTKDPSGPPLDANIFQGKLYPEAKDHPEMKFLHWFRKWRQLHRD |
| amino | | QEYEVTWYVSWSPCTRCANSVATFLAEDPKVTLTIFVARLYYFWKPDYQQALRILCQERGGPHATMKIMNYNEFQHCWNEFVD |
| acid | | GQGKPFKPRKNLPKHYTLLHATLGELLRHVMDPGTFTSNENNKPWVSGQRETYLCYKVERSHNDTWVLLNQHRGELRNQAPDR |
| sequences | | HGFPKGRHAELCFLDLIPFWKLDDQQYRVTCFTSWSPCFSCAQKMAKFISNNKHVSLCIFAARIYDDQGRCQEGLRTLHRDGA |
| for | | KIAVMNYSEFEYCWDTFVDRQGRPFQPWDGLDEHSQALSGRLRAI |
| cytidine | | |
| deaminase | | |
| Exemplary | 189 | MNPQIRNPMERMYRDTFYDNFENEPILYGRSYTWLCYEVKIKRGRSNLLWDTGVERGQVYFKPQYHAEMCFLSWFCGNQLPAY |
| amino | | KCFQITWFVSWTPCPDCVAKLAEFLSEHPNVTLTISAARLYYYWERDYRRALCRLSQAGARVTIMDYEEFAYCWENFVYNEGQ |
| acid | | QFMPWYKEDENYAFLHRTLKEILRYLMDPDTFTENENNDPLVLRRRQTYLCYEVERLDNGTWVLMDQHMGFLCNEAKNLLCGE |
| sequences | | YGRHAELRFLDLVPSLQLDPAQIYRVTWFISWSPCFSWGCAGEVRAFLQENTHVRLRIFAARIYDYDPLYKEALQMLRDAGAQ |
| for | | VSIMTYDEFEYCWDTFVYRQGCPFQPWDGLEEHSQALSGRLRAILQNQGN |
| cytidine | | |
| deaminase | | |
| Exemplary | 190 | MKPQIRNMVEPMDPRTFVSNENNRPILSGLDTVWLCCEVKTKDPSGPPLDAKIFQGKVYPKAKYHPEMRFLRWFHKWRQLHHD |
| amino | | QEYKVTWYVSWSPCTRCANSVATFLAKDPKVTLTIFVARLYYFWKPDYQQALRILCQKRDGPHATMKIMNYNEFQDCWNKFVD |
| acid | | GRGKPFKPWNNLPKHYTLLQATLGELLRHLMDPGTFTSNENNKPWVSGQHETYLCYKVERLHNDTWVPLNQHRGELRNQAPNI |
| sequences | | HGFPKGRHAELCFLDLIPFWKLDGQQYRVTCFTSWSPCESCAQEMAKFISNNEHVSLCIFAARIYDDQGRYQEGLRTLHRDGA |
| for | | KIAMMNYSEFEYCWDTFVDRQGRPFQPWDGLDEHSQALSGRLRAILQNQGN |
| cytidine | | |
| deaminase | | |
| Exemplary | 191 | MKPHFRNTVERMYRDTFSYNFYNRPILSRRNTVWLCYEVKTKGPSRPPLDAKIFRGQVYSELKYHPEMRFFHWFSKWRKLHRD |
| amino | | QEYEVTWYISWSPCTKCTRDMATFLAEDPKVTLTIFVARLYYFWDPDYQEALRSLCQKRDGPRATMKIMNYDEFQHCWSKFVY |
| acid | | SQRELFEPWNNLPKYYILLHIMLGEILRHSMDPPTFTENENNEPWVRGRHETYLCYEVERMHNDTWVLLNQRRGFLCNQAPHK |
| sequences | | HGFLEGRHAELCFLDVIPFWKLDLDQDYRVTCFTSWSPCFSCAQEMAKFISKNKHVSLCIFTARIYDDQGRCQEGLRTLAEAG |
| for | | AKISIMTYSEFKHCWDTFVDHQGCPFQPWDGLDEHSQDLSGRLRAILQNQEN |
| cytidine | | |
| deaminase | | |
| Exemplary | 192 | MKPHERNPVERMYQDTFSDNFYNRPILSHRNTVWLCYEVKTKGPSRPPLDAKIFRGQVYSKLKYHPEMRFFHWFSKWRKLHRD |
| amino | | QEYEVTWYISWSPCTKCTRDVATFLAEDPKVTLTIFVARLYYFWDPDYQEALRSLCQKRDGPRATMKIMNYDEFQHCWSKFVY |
| acid | | SQRELFEPWNNLPKYYILLHIMLGEILRHSMDPPTFTSNENNELWVRGRHETYLCYEVERLHNDTWVLLNQRRGELCNQAPHK |
| sequences | | HGFLEGRHAELCFLDVIPFWKLDLHQDYRVTCFTSWSPCFSCAQEMAKFISNNKHVSLCIFAARIYDDQGRCQEGLRTLAKAG |
| for | | AKISIMTYSEFKHCWDTFVDHQGCPFQPWDGLEEHSQALSGRLRAILQNQGN |
| cytidine | | |
| deaminase | | |
| Exemplary | 193 | MNPQIRNPMERMYRDTFYDNFENEPILYGRSYTWLCYEVKIKRGRSNLLWDTGVFRGPVLPKRQSNHRQEVYFRFENHAEMCF |
| amino | | LSWFCGNRLPANRRFQITWFVSWNPCLPCVVKVTKFLAEHPNVTLTISAARLYYYRDRDWRWVLLRLHKAGARVKIMDYEDFA |
| acid | | YCWENFVCNEGQPFMPWYKFDDNYASLHRTLKEILRNPMEAMYPHIFYFHFKNLLKACGRNESWLCFTMEVTKHHSAVERKRG |
| sequences | | VFRNQVDPETHCHAERCFLSWFCDDILSPNTNYEVTWYTSWSPCPECAGEVAEFLARHSNVNLTIFTARLCYFWDTDYQEGLC |
| for | | SLSQEGASVKIMGYKDFVSCWKNFVYSDDEPFKPWKGLQTNERLLKRRLREILQ |
| cytidine | | |
| deaminase | | |
| Exemplary | 194 | MQPQRLGPRAGMGPFCLGCSHRKCYSPIRNLISQETFKFHFKNLGYAKGRKDTFLCYEVTRKDCDSPVSLHHGVFKNKDNIHA |
| amino | | EICFLYWFHDKVLKVLSPREEFKITWYMSWSPCFECAEQIVRFLATHHNLSLDIFSSRLYNVQDPETQQNLCRLVQEGAQVAA |
| acid | | MDLYEFKKCWKKFVDNGGRRFRPWKRLLTNFRYQDSKLQEILRRMDPLSEEEFYSQFYNQRVKHLCYYHRMKPYLCYQLEQFN |
| sequences | | GQAPLKGCLLSEKGKQHAEILFLDKIRSMELSQVTITCYLTWSPCPNCAWQLAAFKRDRPDLILHIYTSRLYFHWKRPFQKGL |
| for | | CSLWQSGILVDVMDLPQFTDCWTNFVNPKRPFWPWKGLEIISRRTQRRLRRIKESRSHAS |
| cytidine | | |
| deaminase | | |
| Exemplary | 195 | MQPQRLGPRAGMGPFCLGCSHRKCYSPIRNLISQETFKFHFKNLGYAKGRKDTFLCYEVTRKDCDSPVSLHHGVFKNKDNIHA |
| amino | | EICFLYWFHDKVLKVLSPREEFKITWYMSWSPCFECAEQIVRFLATHHNLSLDIFSSRLYNVQDPETQQNLCRLVQEGAQVAA |
| acid | | MDLYEFKKCWKKFVDNGGRRFRPWKRLLTNFRYQDSKLQEILRRMDPLSEEEFYSQFYNQRVKHLCYYHRMKPYLCYQLEQEN |
| sequences | | GQAPLKGCLLSEKGKQHAEILFLDKIRSMELSQVTITCYLTWSPCPNCAWQLAAFKRDRPDLILHIYTSRLYFHWKRPFQKGL |
| for | | CSLWQSGILVDVMDLPQFTDCWTNFVNPKRPFWPWKGLEIISRRTQRRLRRIKESWGLQDLVNDEGNLQLGPPMS |
| cytidine | | |
| deaminase | | |
| Exemplary | 196 | MGPFCLGCSHRKCYSPIRNLISQETFKFHFKNLGYAKGRKDTFLCYEVTRKDCDSPVSLHHGVFKNKDNIHAEICFLYWFHDK |
| amino | | VLKVLSPREEFKITWYMSWSPCFECAEQIVRFLATHHNLSLDIFSSRLYNVQDPETQQNLCRLVQEGAQVAAMDLYEFKKCWK |
| acid | | KFVDNGGRRFRPWKRLLTNFRYQDSKLQEILRPCYISVPSSSSSTLSNICLTKGLPETRFWVEGRRMDPLSEEEFYSQFYNQR |
| sequences | | VKHLCYYHRMKPYLCYQLEQFNGQAPLKGCLLSEKGKQHAEILFLDKIRSMELSQVTITCYLTWSPCPNCAWQLAAFKRDRPD |
| for | | LILHIYTSRLYFHWKRPFQKGLCSLWQSGILVDVMDLPQFTDCWTNFVNPKRPFWPWKGLEIISRRTQRRLRRIKESWGLQDL |
| cytidine | | VNDFGNLQLGPPMS |
| deaminase | | |
| Exemplary | 197 | MGPFCLGCSHRKCYSPIRNLISQETFKFHFKNLGYAKGRKDTFLCYEVTRKDCDSPVSLHHGVFKNKDNIHAEICFLYWFHDK |
| amino | | VLKVLSPREEFKITWYMSWSPCFECAEQIVRFLATHHNLSLDIFSSRLYNVQDPETQQNLCRLVQEGAQVAAMDLYEFKKCWK |
| acid | | KFVDNGGRRFRPWKRLLTNFRYQDSKLQEILRPCYIPVPSSSSSTLSNICLTKGLPETRFCVEGRRMDPLSEEEFYSQFYNQR |
| sequences | | VKHLCYYHRMKPYLCYQLEQFNGQAPLKGCLLSEKGKQHAEILFLDKIRSMELSQVTITCYLTWSPCPNCAWQLAAFKRDRPD |
| for | | LILHIYTSRLYFHWKRPFQKGLCSLWQSGILVDVMDLPQFTDCWTNFVNPKRPFWPWKGLEIISRRTQRRLRRIKESWGLQDL |
| cytidine | | VNDFGNLQLGPPMS |
| deaminase | | |
| Exemplary | 198 | MQPQRLGPRAGMGPFCLGCSHRKCYSPIRNLISQETFKFHFKNLGYAKGRKDTFLCYEVTRKDCDSPVSLHHGVEKNKDNIHA |
| amino | | EICFLYWFHDKVLKVLSPREEFKITWYMSWSPCFECAEQIVRFLATHHNLSLDIFSSRLYNVQDPETQQNLCRLVQEGAQVAA |
| acid | | MDLYEFKKCWKKFVDNGGRRFRPWKRLLTNFRYQDSKLQEILRPCYISVPSSSSSTLSNICLTKGLPETRFWVEGRRMDPLSE |
| sequences | | EEFYSQFYNQRVKHLCYYHRMKPYLCYQLEQFNGQAPLKGCLLSEKGKQHAEILFLDKIRSMELSQVTITCYLTWSPCPNCAW |
| for | | QLAAFKRDRPDLILHIYTSRLYFHWKRPFQKGLCSLWQSGILVDVMDLPQFTDCWTNFVNPKRPFWPWKGLEIISRRTQRRLR |
| cytidine | | RIKEVRTTLLQGPAS |
| deaminase | | |
| Exemplary | 199 | MQPQRLGPRAGMGPFCLGCSHRKCYSPIRNLISQETFKFHFKNLGYAKGRKDTFLCYEVTRKDCDSPVSLHHGVEKNKDNIHA |
| amino | | EICFLYWFHDKVLKVLSPREEFKITWYMSWSPCFECAEQIVRFLATHHNLSLDIFSSRLYNVQDPETQQNLCRLVQEGAQVAA |
| acid | | MDLYEFKKCWKKFVDNGGRRFRPWKRLLTNFRYQDSKLQEILRPCYISVPSSSSSTLSNICLTKGLPETRFWVEGRRMDPLSE |
| sequences | | EEFYSQFYNQRVKHLCYYHRMKPYLCYQLEQFNGQAPLKGCLLSEKGKQHAEILELDKIRSMELSQVTITCYLTWSPCPNCAW |
| for | | QLAAFKRDRPDLILHIYTSRLYFHWKRPFQKGLCSLWQSGILVDVMDLPQFTDCWTNFVNPKRPFWPWKGLEIISRRTQRRLR |
| cytidine | | RIKESWGLQDLVNDFGNLQLGPPMS |
| deaminase | | |
| Exemplary | 200 | MKETDQMQSLEGSGAERSVGTQTGSMTGQIPRLSKVNLFTLLSLWMELFPGVEAQGQKSQKTEEESRGPLGDNEELTRVSTEK |
| amino | | KQVKKTGLVVVKNMKIIGLHCSSEDLHTGQIALIKHGSRLKNCDLYESRKPCSACLKMIVNAGVNRISYWPSDPEISLLTEAS |
| acid | | SSEDAKLDAKAAERLKSNSRAHVCVLLQPLVCYMVQFVEETSYKCDFIQKTAKALPGADTDFYSECKQERIKEYEMLFLVSNE |
| sequences | | ERHKQILMTIGLESLCEDPYESNLRQNMKDLILLLATVASSVPNLKHFGFYCSSPEQINEIHNQSLPQEVARHCMVQARLLAY |
| for | | RTEDHKTGVGAVIWAEAKSRSCDGTGAMYFIGCGYNAFPVGSEYADEPHMDDKHKDREIRKFRYIIHAEQNALTFRCQDIKPE |
| cytidine | | ERSMIFVTKCPCDECVPLIKGAGIKQIYAGDVDVGKKKADISYMKFGELEGVRKFTWQLNPSEAYSLDPNEPERRENGVLRRR |
| deaminase | | SAKDEQRSSKRPRLETRSAGSATTACF |
| Exemplary | 201 | MSNNALQTIINARLPGEEGLWQIHLQDGKISAIDAQSGVMPITENSLDAEQGLVIPPFVEPHIHLDTTQTAGQPNWNQSGTLF |
| amino | | EGIERWAERKALLTHDDVKQRAWQTLKWQIANGIQHVRTHVDVSDATLTALKAMLEVKQEVAPWIDLQIVAFPQEGILSYPNG |
| acid | | EALLEEALRLGADVVGAIPHFEFTREYGVESLHKTFALAQKYDRLIDVHCDEIDDEQSREVETVAALAHHEGMGARVTASHTT |
| sequences | | AMHSYNGAYTSRLFRLLKMSGINFVANPLVNIHLQGREDTYPKRRGITRVKEMLESGINVCFGHDDVFDPWYPLGTANMLQVL |
| for | | HMGLHVCQLMGYGQINDGLNLITHHSARTLNLQDYGIAAGNSANLIILPAENGEDALRRQVPVRYSVRGGKVIASTQPAQTTV |
| cytidine | | YLEQPEAIDYKR |
| deaminase | | |
| Exemplary | 202 | MALLTAKTFSLQFNNKRRVNKPYYPRKALLCYQLTPQNGSTPTRGHLKNKKKDHAEIRFINKIKSMGLDETQCYQVTCYLTWS |
| amino | | PCPSCAGELVDFIKAHRHLNLRIFASRLYYHWRPNYQEGLLLLCGSQVPVEVMGLPEFTDCWENFVDHKEPPSFNPSEKLEEL |
| acid | | DKNSQAIKRRLERIKSRSVDVLENGLRSLQLGPVTPSSSIRNSR |
| sequences | | |
| for | | |
| cytidine | | |
| deaminase | | |
| Exemplary | 203 | MEKDINLKIFKGNLIFTKTSDKFTIMKDSYIVVIDGKIASVSSNLPDKYKGNPIIDERNNIIIPGMNDLHAHASQYKNLGIGM |
| amino | | DKELLPWLNNYTFPEEAKFLNVDYAKKTYGRLIKDLIKNGTTRVALFATLHKDSTIELFNMLIKSGIGAYVGKVNMDYNCPDY |
| acid | | LTENYITSLNDTEEIILKYKDKSNIVKPIITPRFVPSCSNELMDGLGKLSYKYRLPVQSHLSENLDEIAVVKSLHKKSNFYGE |
| sequences | | VYDKFGLFGNTPTLMAHCIHSSKEEINLIKRNNVTIVHCPTSNENLGSGMMPVRKYLNLGINVVLGSDISAGHTCSLEKVIAY |
| for | | AIQNSKIKWQESGKKDMFLSTSEAFYMATKKGGSFFGKVGSFEEGYDEDALVINDSNLYPEDYDLTERLERFIYLGDDRNIMK |
| cytidine | | RYVCGNEIFGPKF |
| deaminase | | |
| Exemplary | 204 | MKIINARLRRQEALFTLDLQDGIIHRITAQAAMQTADAGAIDAQGRLAIPPFVEPHIHLDATLTAGEPEWNRSGTLFEGITRW |
| amino | | SQRKASITPEDTRQRALKTIGMLRDFGVQHVRTHVDVTDPSLAALQALLAVKQEAADLIDLQIVAFPQEGIESYPNGRELMTR |
| acid | | AIEMGADVVGGIPHYENTRDKGVSSVMFLMDLAQRYGRLVDVHCDEIDDPQSRELEVLAEEARVRGMGAQVTASHTCAMGSYD |
| sequences | | NAYCSKLERLLKASGINFISCPTESIHLQGRFDSWPKRRGVTRVAELDRAGINVCFAQDSIQDPWYPLGNGNILRILDAGLHI |
| for | | CHMLGYDDLQRCLDFVTDNSARALCLGDNYGLAEGRPANLLILDAENDYEAVRRQARVLTSIRHGKVILQREVEHIRYPA |
| cytidine | | |
| deaminase | | |
| Exemplary | 205 | MGRKLDPTKEKRGPGRKARKQKGAETELVRELPAVSDENSKRLSSRARKRAAKRRLGSVEAPKTNKSPEAKPLPGKLPKGISA |
| amino | | GAVQTAGKKGPQSLFNAPRGKKRPAPGSDEEEEEEDSEEDGMVNHGDLWGSEDDADTVDDYGADSNSEDEEEGEALLPIERAA |
| acid | | RKQKAREAAAGIQWSEEETEDEEEEKEVTPESGPPKVEEADGGLQINVDEEPFVLPPAGEMEQDAQAPDLQRVHKRIQDIVGI |
| sequences | | LRDFGAQREEGRSRSEYLNRLKKDLAIYYSYGDFLLGKLMDLFPLSELVEFLEANEVPRPVTLRTNTLKTRRRDLAQALINRG |
| for | | VNLDPLGKWSKTGLVVYDSSVPIGATPEYLAGHYMLQGASSMLPVMALAPQEHERILDMCCAPGGKTSYMAQLMKNTGVILAN |
| cytidine | | DANAERLKSVVGNLHRLGVINTIISHYDGRQFPKVVGGFDRVLLDAPCSGTGVISKDPAVKTNKDEKDILRCAHLQKELLLSA |
| deaminase | | IDSVNATSKTGGYLVYCTCSITVEENEWVVDYALKKRNVRLVPTGLDFGQEGFTRFRERRFHPSLRSTRRFYPHTHNMDGFFI |
| | | AKFKKFSNSIPQSQTGNSETATPTNVDLPQVIPKSENSSQPAKKAKGAAKTKQQLQKQQHPKKASFQKLNGISKGADSELSTV |
| | | PSVTKTQASSSFQDSSQPAGKAEGIREPKVTGKLKQRSPKLQSSKKVAFLRQNAPPKGTDTQTPAVLSPSKTQATLKPKDHHQ |
| | | PLGRAKGVEKQQLPEQPFEKAAFQKQNDTPKGPQPPTVSPIRSSRPPPAKRKKSQSRGNSQLLLS |
| Exemplary | 206 | MGRRSRGRRLQQQQRPEDAEDGAEGGGKRGEAGWEGGYPEIVKENKLFEHYYQELKIVPEGEWGQEMDALREPLPATLRITGY |
| amino | | KSHAKEILHCLKNKYFKELEDLEVDGQKVEVPQPLSWYPEELAWHTNLSRKILRKSPHLEKFHQFLVSETESGNISRQEAVSM |
| acid | | IPPLLLNVRPHHKILDMCAAPGSKTTQLIEMLHADMNVPFPEGEVIANDVDNKRCYLLVHQAKRLSSPCIMVVNHDASSIPRL |
| sequences | | QIDVDGRKEILFYDRILCDVPCSGDGTMRKNIDVWKKWTTLNSLQLHGLQLRIATRGAEQLAEGGRMVYSTCSLNPIEDEAVI |
| for | | ASLLEKSEGALELADVSNELPGLKWMPGITQWKVMTKDGQWFTDWDAVPHSRHTQIRPTMFPPKDPEKLQAMHLERCLRILPH |
| cytidine | | HQNTGGFFVAVLVKKSSMPWNKRQPKLQGKSAETRESTQLSPADLTEGKPTDPSKLESPSFTGTGDTEIAHATEDLENNGSKK |
| deaminase | | DGVCGPPPSKKMKLFGFKEDPFVFIPEDDPLFPPIEKFYALDPSFPRMNLLTRTTEGKKRQLYMVSKELRNVLLNNSEKMKVI |
| | | NTGIKVWCRNNSGEEFDCAFRLAQEGIYTLYPFINSRIITVSMEDVKILLTQENPFFRKLSSETYSQAKDLAKGSIVLKYEPD |
| | | SANPDALQCPIVLCGWRGKASIRTFVPKNERLHYLRMMGLEVLGEKKKEGVILTNESAASTGQPDNDVTEGQRAGEPNSPDAE |
| | | EANSPDVTAGCDPAGVHPPR^{∧}p |
| Exemplary | 207 | MGPFCLGCSHRKCYSPIRNLISQETFKFHFKNLGYAKGRKDTFLCYEVTRKDCDSPVSLHHGVFKNKDNIHAEICFLYWFHDK |
| amino | | VLKVLSPREEFKITWYMSWSPCFECAEQIVRFLATHHNLSLDIFSSRLYNVQDPETQQNLCRLVQEGAQVAAMDLYEFKKCWK |
| acid | | KFVDNGGRRFRPWKRLLTNFRYQDSKLQEILRPCYIPVPSSSSSTLSNICLTKGLPETRFCVEGRRMDPLSEEEFYSQFYNQR |
| sequences | | VKHLCYYHRMKPYLCYQLEQFNGQAPLKGCLLSEKGKQHAEILELDKIRSMELSQVTITCYLTWSPCPNCAWQLAAFKRDRPD |
| for | | LILHIYTSRLYFHWKRPFQKGLCSLWQSGILVDVMDLPQFTDCWTNFVNPKRPFWPWKGLEIISRRTQRRLRRIKESWGLQDL |
| cytidine | | VNDFGNLQLGPPMS |
| deaminase | | |
| Exemplary | 208 | MGPFCLGCSHRKCYSPIRNLISQETFKFHFKNLRYAIDRKDTFLCYEVTRKDCDSPVSLHHGVFKNKDNIHAEICFLYWFHDK |
| amino | | VLKVLSPREEFKITWYMSWSPCFECAEQVLRFLATHHNLSLDIFSSRLYNIRDPENQQNLCRLVQEGAQVAAMDLYEFKKCWK |
| acid | | KFVDNGGRRFRPWKKLLTNFRYQDSKLQEILRPCYIPVPSSSSSTLSNICLTKGLPETRFCVERRRVHLLSEEEFYSQFYNQR |
| sequences | | VKHLCYYHGVKPYLCYQLEQFNGQAPLKGCLLSEKGKQHAEILFLDKIRSMELSQVIITCYLTWSPCPNCAWQLAAFKRDRPD |
| for | | LILHIYTSRLYFHWKRPFQKGLCSLWQSGILVDVMDLPQFTDCWTNFVNPKRPFWPWKGLEIISRRTQRRLHRIKESWGLQDL |
| cytidine | | VNDFGNLQLGPPMS |
| deaminase | | |
| Exemplary | 209 | MVEPMDPRTFVSNFNNRPILSGLNTVWLCCEVKTKDPSGPPLDAKIFQGKVYSKAKYHPEMRFLRWFHKWRQLHHDQEYKVTW |
| amino | | YVSWSPCTRCANSVATFLAKDPKVTLTIFVARLYYFWKPDYQQALRILCQKRGGPHATMKIMNYNEFQDCWNKFVDGRGKPEK |
| acid | | PRNNLPKHYTLLQATLGELLRHLMDPGTFTSNENNKPWVSGQHETYLCYKVERLHNDTWVPLNQHRGELRNQAPNIHGFPKGR |
| sequences | | HAELCFLDLIPFWKLDGQQYRVTCFTSWSPCFSCAQEMAKFISNNEHVSLCIFAARIYDDQGRYQEGLRALHRDGAKIAMMNY |
| for | | SEFEYCWDTFVDRQGRPFQPWDGLDEHSQALSGRLRAI |
| cytidine | | |
| deaminase | | |
| Exemplary | 210 | MKPHERNPVERMYQDTFSDNFYNRPILSHRNTVWLCYEVKTKGPSRPPLDAKIFRGQVYSKLKYHPEMRFFHWFSKWRKLHRD |
| amino | | QEYEVTWYISWSPCTKCTRDVATFLAEDPKVTLTIFVARLYYFWDPDYQEALRSLCQKRDGPRATMKIMNYDEFQHCWSKEVY |
| acid | | SQRELFEPWNNLPKYYILLHIMLGEILRHSMDPPTFTSNENNELWVRGRHETYLCYEVERLHNDTWVLLNQRRGFLCNQAPHK |
| sequences | | HGFLEGRHAELCFLDVIPFWKLDLHQDYRVTCFTSWSPCFSCAQEMAKFISNNKHVSLCIFAARIYDDQGRCQEGLRTLAKAG |
| for | | AKISIMTYSEFKHCWDTFVDHQGCPFQPWDGLEEHSQALSGRLRAILQNQGN |
| cytidine | | |
| deaminase | | |
| Exemplary | 211 | MNPQIRNMVEQMEPDIFVYYENNRPILSGRNTVWLCYEVKTKDPSGPPLDANIFQGKLYPEAKDHPEMKFLHWFRKWRQLHRD |
| amino | | QEYEVTWYVSWSPCTRCANSVATFLAEDPKVTLTIFVARLYYFWKPDYQQALRILCQERGGPHATMKIMNYNEFQHCWNEFVD |
| acid | | GQGKPFKPRKNLPKHYTLLHATLGELLRHVMDPGTFTSNENNKPWVSGQRETYLCYKVERSHNDTWVLLNQHRGFLRNQAPDR |
| sequences | | HGFPKGRHAELCFLDLIPFWKLDDQQYRVTCFTSWSPCFSCAQKMAKFISNNKHVSLCIFAARIYDDQGRCQEGLRTLHRDGA |
| for | | KIAVMNYSEFEYCWDTFVDRQGRPFQPWDGLDEHSQALSGRLRAI |
| cytidine | | |
| deaminase | | |
| Exemplary | 212 | MKPHFRNTVERMYRDTFSYNFYNRPILSRRNTVWLCYEVKTKGPSRPPLDAKIFRGQVYSELKYHPEMRFFHWFSKWRKLHRD |
| amino | | QEYEVTWYISWSPCTKCTRDMATFLAEDPKVTLTIFVARLYYFWDPDYQEALRSLCQKRDGPRATMKIMNYDEFQHCWSKFVY |
| acid | | SQRELFEPWNNLPKYYILLHIMLGEILRHSMDPPTFTENENNEPWVRGRHETYLCYEVERMHNDTWVLLNQRRGFLCNQAPHK |
| sequences | | HGFLEGRHAELCFLDVIPFWKLDLDQDYRVTCFTSWSPCFSCAQEMAKFISKNKHVSLCIFTARIYDDQGRCQEGLRTLAEAG |
| for | | AKISIMTYSEFKHCWDTFVDHQGCPFQPWDGLDEHSQDLSGRLRAILQNQEN |
| cytidine | | |
| deaminase | | |
| Exemplary | 213 | MKPHFRNTVERMYRDTFSYNFYNRPILSRRNTVWLCYEVKTKGPSRPRLDAKIFRGQVYSQPEHHAEMCFLSWFCGNQLPAYK |
| amino | | CFQITWFVSWTPCPDCVAKLAEFLAEHPNVTLTISAARLYYYWERDYRRALCRLSQAGARVKIMDDEEFAYCWENFVYSEGQP |
| acid | | FMPWYKFDDNYAFLHRTLKEILRNPMEAMYPHIFYFHFKNLRKAYGRNESWLCFTMEVVKHHSPVSWKRGVFRNQVDPETHCH |
| sequences | | AERCFLSWFCDDILSPNTNYEVTWYTSWSPCPECAGEVAEFLARHSNVNLTIFTARLYYFWDTDYQEGLRSLSQEGASVEIMG |
| for | | YKDFKYCWENFVYNDDEPFKPWKGLKYNFLFLDSKLQEILE |
| cytidine | | |
| deaminase | | |
| Exemplary | 214 | MNPQIRNPMERMYRDTFYDNFENEPILYGRSYTWLCYEVKIKRGRSNLLWDTGVFRGQVYFKPQYHAEMCFLSWFCGNQLPAY |
| amino | | KCFQITWFVSWTPCPDCVAKLAEFLSEHPNVTLTISAARLYYYWERDYRRALCRLSQAGARVTIMDYEEFAYCWENFVYNEGQ |
| acid | | QFMPWYKFDENYAFLHRTLKEILRYLMDPDTFTENENNDPLVLRRRQTYLCYEVERLDNGTWVLMDQHMGFLCNEAKNLLCGF |
| sequences | | YGRHAELRFLDLVPSLQLDPAQIYRVTWFISWSPCFSWGCAGEVRAFLQENTHVRLRIFAARIYDYDPLYKEALQMLRDAGAQ |
| for | | VSIMTYDEFEYCWDTFVYRQGCPFQPWDGLEEHSQALSGRLRAILQNQGN |
| cytidine | | |
| deaminase | | |
| Exemplary | 215 | MNPQIRNPMERMYRDTFYDNFENEPILYGRSYTWLCYEVKIKRGRSNLLWDTGVFRGPVLPKRQSNHRQEVYFRFENHAEMCF |
| amino | | LSWFCGNRLPANRRFQITWFVSWNPCLPCVVKVTKFLAEHPNVTLTISAARLYYYRDRDWRWVLLRLHKAGARVKIMDYEDFA |
| acid | | YCWENFVCNEGQPFMPWYKEDDNYASLHRTLKEILRNPMEAMYPHIFYFHFKNLLKACGRNESWLCFTMEVTKHHSAVERKRG |
| sequences | | VFRNQVDPETHCHAERCFLSWFCDDILSPNTNYEVTWYTSWSPCPECAGEVAEFLARHSNVNLTIFTARLCYFWDTDYQEGLC |
| for | | SLSQEGASVKIMGYKDFVSCWKNFVYSDDEPFKPWKGLQTNERLLKRRLREILQ |
| cytidine | | |
| deaminase | | |
| Exemplary | 216 | MSSETGPVAVDPTLRRRIEPHEFEVFFDPRELRKETCLLYEINWGGRHSIWRHTSQNTNKHVEVNFIEKFTTERYFCPNTRCS |
| amino | | ITWFLSWSPCGECSRAITEFLSRYPHVTLFIYIARLYHHADPRNRQGLRDLISSGVTIQIMTEQESGYCWRNEVNYSPSNEAH |
| acid | | WPRYPHLWVRLYVL ELYCIILGLPPCLNILRRKQPQLTFFTIALQSCHYQRLPPHILWATGLK |
| sequences | | |
| for | | |
| cytidine | | |
| deaminase | | |
| Exemplary | 220 | MAAFKPNSINYILGLDIGIASVGWAMVEIDEEENPIRLIDLGVRVFERAEVPKTGDSLAMARRLARSVRRLTRRRAHRLLRTR |
| amino | | RLLKREGVLQAANEDENGLIKSLPNTPWQLRAAALDRKLTPLEWSAVLLHLIKHRGYLSQRKNEGETADKELGALLKGVAGNA |
| acid | | HALQTGDERTPAELALNKFEKESGHIRNQRSDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSPELQDEIGTAFSLFKTDEDITGRLKDRIQPEILEALLKHI |
| Nme1Cas9 | | SFDKFVQISLKALRRIVPLMEQGKRYDEACAEIYGDHYGKKNTEEKIYLPPIPADEIRNPVVLRALSQARKVINGVVRRYGSP |
| cleavase | | ARIHIETAREVGKSFKDRKEIEKRQEENRKDREKAAAKFREYFPNFVGEPKSKDILKLRLYEQQHGKCLYSGKEINLGRLNEK |
| | | GYVEIDHALPFSRTWDDSFNNKVLVLGSENQNKGNQTPYEYENGKDNSREWQEFKARVETSRFPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVQGKDEEDWQLIDDSFN |
| | | FKFSLHPNDLVEVITKKARMFGYFASCHRGTGNINIRIHDLDHKIGKNGILEGIGVKTALSFQKYQIDELGKEIRPCRLKKRP |
| | | PVR |
| Exemplary | 221 | GCTGCTTTTAAGCCTAATTCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTACTCGTCGT |
| encoding | | CTTCTTAAGCGTGAGGGTGTTCTTCAGGCTGCTAATTTTGATGAGAATGGTCTTATTAAGTCTCTTCCTAATACTCCTTGGCA |
| Nme1Cas9 | | GCTTCGTGCTGCTGCTCTTGATCGTAAGCTTACTCCTCTTGAGTGGTCTGCTGTTCTTCTTCATCTTATTAAGCATCGTGGTT |
| cleavase | | ATCTTTCTCAGCGTAAGAATGAGGGTGAGACTGCTGATAAGGAGCTTGGTGCTCTTCTTAAGGGTGTTGCTGGTAATGCTCAT |
| | | GCTCTTCAGACTGGTGATTTTCGTACTCCTGCTGAGCTTGCTCTTAATAAGTTTGAGAAGGAGTCTGGTCATATTCGTAATCA |
| | | GCGTTCTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCTTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTCAGGGTAAGGATGAGGAGGATTGGCAGCTTATTGATGATTCTTTTAATTTT |
| | | AAGTTTTCTCTTCATCCTAATGATCTTGTTGAGGTTATTACTAAGAAGGCTCGTATGTTTGGTTATTTTGCTTCTTGTCATCG |
| | | TGGTACTGGTAATATTAATATTCGTATTCATGATCTTGATCATAAGATTGGTAAGAATGGTATTCTTGAGGGTATTGGTGTTA |
| | | AGACTGCTCTTTCTTTTCAGAAGTATCAGATTGATGAGCTTGGTAAGGAGATTCGTCCTTGTCGTCTTAAGAAGCGTCCTCCT |
| | | GTTCGT |
| Exemplary | 222 | GCCGCCTTCAAGCCCAACTCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGACCCGGCGG |
| encoding | | CTGCTGAAGCGGGAGGGCGTGCTGCAGGCCGCCAACTTCGACGAGAACGGCCTGATCAAGTCCCTGCCCAACACCCCCTGGCA |
| Nme1Cas 9 | | GCTGCGGGCCGCCGCCCTGGACCGGAAGCTGACCCCCCTGGAGTGGTCCGCCGTGCTGCTGCACCTGATCAAGCACCGGGGCT |
| cleavase | | ACCTGTCCCAGCGGAAGAACGAGGGCGAGACCGCCGACAAGGAGCTGGGCGCCCTGCTGAAGGGCGTGGCCGGCAACGCCCAC |
| | | GCCCTGCAGACCGGCGACTTCCGGACCCCCGCCGAGCTGGCCCTGAACAAGTTCGAGAAGGAGTCCGGCCACATCCGGAACCA |
| | | GCGGTCCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCTGG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGCAGGGCAAGGACGAGGAGGACTGGCAGCTGATCGACGACTCCTTCAACTTC |
| | | AAGTTCTCCCTGCACCCCAACGACCTGGTGGAGGTGATCACCAAGAAGGCCCGGATGTTCGGCTACTTCGCCTCCTGCCACCG |
| | | GGGCACCGGCAACATCAACATCCGGATCCACGACCTGGACCACAAGATCGGCAAGAACGGCATCCTGGAGGGCATCGGCGTGA |
| | | AGACCGCCCTGTCCTTCCAGAAGTACCAGATCGACGAGCTGGGCAAGGAGATCCGGCCCTGCCGGCTGAAGAAGCGGCCCCCC |
| | | GTGCGG |
| Exemplary | 223 | GCAGCATTCAAACCAAACTCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAACACGACGA |
| encoding | | CTACTAAAACGAGAAGGAGTACTACAAGCAGCAAACTTCGACGAAAACGGACTAATCAAATCACTACCAAACACACCATGACA |
| Nme1Cas9 | | ACTACGAGCAGCAGCACTAGACCGAAAACTAACACCACTAGAATGATCAGCAGTACTACTACACCTAATCAAACACCGAGGAT |
| cleavase | | ACCTATCACAACGAAAAAACGAAGGAGAAACAGCAGACAAAGAACTAGGAGCACTACTAAAAGGAGTAGCAGGAAACGCACAC |
| | | GCACTACAAACAGGAGACTTCCGAACACCAGCAGAACTAGCACTAAACAAATTCGAAAAAGAATCAGGACACATCCGAAACCA |
| | | ACGATCAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACCACGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACTAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTACAAGGAAAAGACGAAGAAGACTGACAACTAATCGACGACTCATTCAACTTC |
| | | AAATTCTCACTACACCCAAACGACCTAGTAGAAGTAATCACAAAAAAAGCACGAATGTTCGGATACTTCGCATCATGCCACCG |
| | | AGGAACAGGAAACATCAACATCCGAATCCACGACCTAGACCACAAAATCGGAAAAAACGGAATCCTAGAAGGAATCGGAGTAA |
| | | AAACAGCACTATCATTCCAAAAATACCAAATCGACGAACTAGGAAAAGAAATCCGACCATGCCGACTAAAAAAACGACCACCA |
| | | GTACGA |
| Exemplary | 224 | ATGGCTGCTTTTAAGCCTAATTCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTACTCGT |
| frame for | | CGTCTTCTTAAGCGTGAGGGTGTTCTTCAGGCTGCTAATTTTGATGAGAATGGTCTTATTAAGTCTCTTCCTAATACTCCTTG |
| Nme1Cas9 | | GCAGCTTCGTGCTGCTGCTCTTGATCGTAAGCTTACTCCTCTTGAGTGGTCTGCTGTTCTTCTTCATCTTATTAAGCATCGTG |
| cleavase | | GTTATCTTTCTCAGCGTAAGAATGAGGGTGAGACTGCTGATAAGGAGCTTGGTGCTCTTCTTAAGGGTGTTGCTGGTAATGCT |
| | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTTCTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | TTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTCAGGGTAAGGATGAGGAGGATTGGCAGCTTATTGATGATTCTTTTAAT |
| | | TTTAAGTTTTCTCTTCATCCTAATGATCTTGTTGAGGTTATTACTAAGAAGGCTCGTATGTTTGGTTATTTTGCTTCTTGTCA |
| | | TCGTGGTACTGGTAATATTAATATTCGTATTCATGATCTTGATCATAAGATTGGTAAGAATGGTATTCTTGAGGGTATTGGTG |
| | | TTAAGACTGCTCTTTCTTTTCAGAAGTATCAGATTGATGAGCTTGGTAAGGAGATTCGTCCTTGTCGTCTTAAGAAGCGTCCT |
| | | CCTGTTCGTUGA |
| Exemplary | 225 | ATGGCCGCCTTCAAGCCCAACTCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGACCCGG |
| frame for | | CGGCTGCTGAAGCGGGAGGGCGTGCTGCAGGCCGCCAACTTCGACGAGAACGGCCTGATCAAGTCCCTGCCCAACACCCCCTG |
| Nme1Cas9 | | GCAGCTGCGGGCCGCCGCCCTGGACCGGAAGCTGACCCCCCTGGAGTGGTCCGCCGTGCTGCTGCACCTGATCAAGCACCGGG |
| cleavase | | GCTACCTGTCCCAGCGGAAGAACGAGGGCGAGACCGCCGACAAGGAGCTGGGCGCCCTGCTGAAGGGCGTGGCCGGCAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGTCCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | TGGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGCAGGGCAAGGACGAGGAGGACTGGCAGCTGATCGACGACTCCTTCAAC |
| | | TTCAAGTTCTCCCTGCACCCCAACGACCTGGTGGAGGTGATCACCAAGAAGGCCCGGATGTTCGGCTACTTCGCCTCCTGCCA |
| | | CCGGGGCACCGGCAACATCAACATCCGGATCCACGACCTGGACCACAAGATCGGCAAGAACGGCATCCTGGAGGGCATCGGCG |
| | | TGAAGACCGCCCTGTCCTTCCAGAAGTACCAGATCGACGAGCTGGGCAAGGAGATCCGGCCCTGCCGGCTGAAGAAGCGGCCC |
| | | CCCGTGCGGUGA |
| Exemplary | 226 | ATGGCAGCATTCAAACCAAACTCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAACACGA |
| frame for | | CGACTACTAAAACGAGAAGGAGTACTACAAGCAGCAAACTTCGACGAAAACGGACTAATCAAATCACTACCAAACACACCATG |
| Nme1Cas9 | | ACAACTACGAGCAGCAGCACTAGACCGAAAACTAACACCACTAGAATGATCAGCAGTACTACTACACCTAATCAAACACCGAG |
| cleavase | | GATACCTATCACAACGAAAAAACGAAGGAGAAACAGCAGACAAAGAACTAGGAGCACTACTAAAAGGAGTAGCAGGAAACGCA |
| | | CACGCACTACAAACAGGAGACTTCCGAACACCAGCAGAACTAGCACTAAACAAATTCGAAAAAGAATCAGGACACATCCGAAA |
| | | CCAACGATCAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACCACGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | TAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTACAAGGAAAAGACGAAGAAGACTGACAACTAATCGACGACTCATTCAAC |
| | | TTCAAATTCTCACTACACCCAAACGACCTAGTAGAAGTAATCACAAAAAAAGCACGAATGTTCGGATACTTCGCATCATGCCA |
| | | CCGAGGAACAGGAAACATCAACATCCGAATCCACGACCTAGACCACAAAATCGGAAAAAACGGAATCCTAGAAGGAATCGGAG |
| | | TAAAAACAGCACTATCATTCCAAAAATACCAAATCGACGAACTAGGAAAAGAAATCCGACCATGCCGACTAAAAAAACGACCA |
| | | CCAGTACGAUAA |
| Exemplary | 227 | MAAFKPNSINYILGLAIGIASVGWAMVEIDEEENPIRLIDLGVRVFERAEVPKTGDSLAMARRLARSVRRLTRRRAHRLLRTR |
| amino | | RLLKREGVLQAANFDENGLIKSLPNTPWQLRAAALDRKLTPLEWSAVLLHLIKHRGYLSQRKNEGETADKELGALLKGVAGNA |
| acid | | HALQTGDFRTPAELALNKFEKESGHIRNQRSDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSPELQDEIGTAFSLFKTDEDITGRLKDRIQPEILEALLKHI |
| Nme1Cas9 | | SFDKFVQISLKALRRIVPLMEQGKRYDEACAEIYGDHYGKKNTEEKIYLPPIPADEIRNPVVLRALSQARKVINGVVRRYGSP |
| dCas9 | | ARIHIETAREVGKSFKDRKEIEKRQEENRKDREKAAAKFREYFPNFVGEPKSKDILKLRLYEQQHGKCLYSGKEINLGRLNEK |
| | | GYVEIDAALPFSRTWDDSFNNKVLVLGSENQNKGNQTPYEYENGKDNSREWQEFKARVETSRFPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVQGKDEEDWQLIDDSFN |
| | | FKFSLHPNDLVEVITKKARMFGYFASCHRGTGNINIRIHDLDHKIGKNGILEGIGVKTALSFQKYQIDELGKEIRPCRLKKRP |
| | | PVR |
| Exemplary | 228 | GCTGCTTTTAAGCCTAATTCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTACTCGTCGT |
| encoding | | CTTCTTAAGCGTGAGGGTGTTCTTCAGGCTGCTAATTTTGATGAGAATGGTCTTATTAAGTCTCTTCCTAATACTCCTTGGCA |
| Nme1Cas9 | | GCTTCGTGCTGCTGCTCTTGATCGTAAGCTTACTCCTCTTGAGTGGTCTGCTGTTCTTCTTCATCTTATTAAGCATCGTGGTT |
| dCas9 | | ATCTTTCTCAGCGTAAGAATGAGGGTGAGACTGCTGATAAGGAGCTTGGTGCTCTTCTTAAGGGTGTTGCTGGTAATGCTCAT |
| | | GCTCTTCAGACTGGTGATTTTCGTACTCCTGCTGAGCTTGCTCTTAATAAGTTTGAGAAGGAGTCTGGTCATATTCGTAATCA |
| | | GCGTTCTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCTTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTCAGGGTAAGGATGAGGAGGATTGGCAGCTTATTGATGATTCTTTTAATTTT |
| | | AAGTTTTCTCTTCATCCTAATGATCTTGTTGAGGTTATTACTAAGAAGGCTCGTATGTTTGGTTATTTTGCTTCTTGTCATCG |
| | | TGGTACTGGTAATATTAATATTCGTATTCATGATCTTGATCATAAGATTGGTAAGAATGGTATTCTTGAGGGTATTGGTGTTA |
| | | AGACTGCTCTTTCTTTTCAGAAGTATCAGATTGATGAGCTTGGTAAGGAGATTCGTCCTTGTCGTCTTAAGAAGCGTCCTCCT |
| | | GTTCGT |
| Exemplary | 229 | GCCGCCTTCAAGCCCAACTCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGACCCGGCGG |
| encoding | | CTGCTGAAGCGGGAGGGCGTGCTGCAGGCCGCCAACTTCGACGAGAACGGCCTGATCAAGTCCCTGCCCAACACCCCCTGGCA |
| Nme1Cas9 | | GCTGCGGGCCGCCGCCCTGGACCGGAAGCTGACCCCCCTGGAGTGGTCCGCCGTGCTGCTGCACCTGATCAAGCACCGGGGCT |
| dCas9 | | ACCTGTCCCAGCGGAAGAACGAGGGCGAGACCGCCGACAAGGAGCTGGGCGCCCTGCTGAAGGGCGTGGCCGGCAACGCCCAC |
| | | GCCCTGCAGACCGGCGACTTCCGGACCCCCGCCGAGCTGGCCCTGAACAAGTTCGAGAAGGAGTCCGGCCACATCCGGAACCA |
| | | GCGGTCCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCTGG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGCAGGGCAAGGACGAGGAGGACTGGCAGCTGATCGACGACTCCTTCAACTTC |
| | | AAGTTCTCCCTGCACCCCAACGACCTGGTGGAGGTGATCACCAAGAAGGCCCGGATGTTCGGCTACTTCGCCTCCTGCCACCG |
| | | GGGCACCGGCAACATCAACATCCGGATCCACGACCTGGACCACAAGATCGGCAAGAACGGCATCCTGGAGGGCATCGGCGTGA |
| | | AGACCGCCCTGTCCTTCCAGAAGTACCAGATCGACGAGCTGGGCAAGGAGATCCGGCCCTGCCGGCTGAAGAAGCGGCCCCCC |
| | | GTGCGG |
| Exemplary | 230 | GCAGCATTCAAACCAAACTCAATCAACTACATCCTAGGACTAGCAATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAACACGACGA |
| encoding | | CTACTAAAACGAGAAGGAGTACTACAAGCAGCAAACTTCGACGAAAACGGACTAATCAAATCACTACCAAACACACCATGACA |
| Nme1Cas9 | | ACTACGAGCAGCAGCACTAGACCGAAAACTAACACCACTAGAATGATCAGCAGTACTACTACACCTAATCAAACACCGAGGAT |
| dCas9 | | ACCTATCACAACGAAAAAACGAAGGAGAAACAGCAGACAAAGAACTAGGAGCACTACTAAAAGGAGTAGCAGGAAACGCACAC |
| | | GCACTACAAACAGGAGACTTCCGAACACCAGCAGAACTAGCACTAAACAAATTCGAAAAAGAATCAGGACACATCCGAAACCA |
| | | ACGATCAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACGCAGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACTAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTACAAGGAAAAGACGAAGAAGACTGACAACTAATCGACGACTCATTCAACTTC |
| | | AAATTCTCACTACACCCAAACGACCTAGTAGAAGTAATCACAAAAAAAGCACGAATGTTCGGATACTTCGCATCATGCCACCG |
| | | AGGAACAGGAAACATCAACATCCGAATCCACGACCTAGACCACAAAATCGGAAAAAACGGAATCCTAGAAGGAATCGGAGTAA |
| | | AAACAGCACTATCATTCCAAAAATACCAAATCGACGAACTAGGAAAAGAAATCCGACCATGCCGACTAAAAAAACGACCACCA |
| | | GTACGA |
| Exemplary | 231 | ATGGCTGCTTTTAAGCCTAATTCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTACTCGT |
| frame for | | CGTCTTCTTAAGCGTGAGGGTGTTCTTCAGGCTGCTAATTTTGATGAGAATGGTCTTATTAAGTCTCTTCCTAATACTCCTTG |
| Nme1Cas9 | | GCAGCTTCGTGCTGCTGCTCTTGATCGTAAGCTTACTCCTCTTGAGTGGTCTGCTGTTCTTCTTCATCTTATTAAGCATCGTG |
| dCas9 | | GTTATCTTTCTCAGCGTAAGAATGAGGGTGAGACTGCTGATAAGGAGCTTGGTGCTCTTCTTAAGGGTGTTGCTGGTAATGCT |
| | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTTCTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | TTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTCAGGGTAAGGATGAGGAGGATTGGCAGCTTATTGATGATTCTTTTAAT |
| | | TTTAAGTTTTCTCTTCATCCTAATGATCTTGTTGAGGTTATTACTAAGAAGGCTCGTATGTTTGGTTATTTTGCTTCTTGTCA |
| | | TCGTGGTACTGGTAATATTAATATTCGTATTCATGATCTTGATCATAAGATTGGTAAGAATGGTATTCTTGAGGGTATTGGTG |
| | | TTAAGACTGCTCTTTCTTTTCAGAAGTATCAGATTGATGAGCTTGGTAAGGAGATTCGTCCTTGTCGTCTTAAGAAGCGTCCT |
| | | CCTGTTCGTUGA |
| Exemplary | 232 | ATGGCCGCCTTCAAGCCCAACTCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGACCCGG |
| frame for | | CGGCTGCTGAAGCGGGAGGGCGTGCTGCAGGCCGCCAACTTCGACGAGAACGGCCTGATCAAGTCCCTGCCCAACACCCCCTG |
| Nme1Cas9 | | GCAGCTGCGGGCCGCCGCCCTGGACCGGAAGCTGACCCCCCTGGAGTGGTCCGCCGTGCTGCTGCACCTGATCAAGCACCGGG |
| dCas9 | | GCTACCTGTCCCAGCGGAAGAACGAGGGCGAGACCGCCGACAAGGAGCTGGGCGCCCTGCTGAAGGGCGTGGCCGGCAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGTCCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | TGGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGCAGGGCAAGGACGAGGAGGACTGGCAGCTGATCGACGACTCCTTCAAC |
| | | TTCAAGTTCTCCCTGCACCCCAACGACCTGGTGGAGGTGATCACCAAGAAGGCCCGGATGTTCGGCTACTTCGCCTCCTGCCA |
| | | CCGGGGCACCGGCAACATCAACATCCGGATCCACGACCTGGACCACAAGATCGGCAAGAACGGCATCCTGGAGGGCATCGGCG |
| | | TGAAGACCGCCCTGTCCTTCCAGAAGTACCAGATCGACGAGCTGGGCAAGGAGATCCGGCCCTGCCGGCTGAAGAAGCGGCCC |
| | | CCCGTGCGGUGA |
| Exemplary | 233 | ATGGCAGCATTCAAACCAAACTCAATCAACTACATCCTAGGACTAGCAATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAACACGA |
| frame for | | CGACTACTAAAACGAGAAGGAGTACTACAAGCAGCAAACTTCGACGAAAACGGACTAATCAAATCACTACCAAACACACCATG |
| Nme1Cas9 | | ACAACTACGAGCAGCAGCACTAGACCGAAAACTAACACCACTAGAATGATCAGCAGTACTACTACACCTAATCAAACACCGAG |
| dCas9 | | GATACCTATCACAACGAAAAAACGAAGGAGAAACAGCAGACAAAGAACTAGGAGCACTACTAAAAGGAGTAGCAGGAAACGCA |
| | | CACGCACTACAAACAGGAGACTTCCGAACACCAGCAGAACTAGCACTAAACAAATTCGAAAAAGAATCAGGACACATCCGAAA |
| | | CCAACGATCAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACGCAGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | TAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTACAAGGAAAAGACGAAGAAGACTGACAACTAATCGACGACTCATTCAAC |
| | | TTCAAATTCTCACTACACCCAAACGACCTAGTAGAAGTAATCACAAAAAAAGCACGAATGTTCGGATACTTCGCATCATGCCA |
| | | CCGAGGAACAGGAAACATCAACATCCGAATCCACGACCTAGACCACAAAATCGGAAAAAACGGAATCCTAGAAGGAATCGGAG |
| | | TAAAAACAGCACTATCATTCCAAAAATACCAAATCGACGAACTAGGAAAAGAAATCCGACCATGCCGACTAAAAAAACGACCA |
| | | CCAGTACGAUAA |
| Exemplary | 234 | MAAFKPNSINYILGLAIGIASVGWAMVEIDEEENPIRLIDLGVRVFERAEVPKTGDSLAMARRLARSVRRLTRRRAHRLLRTR |
| amino | | RLLKREGVLQAANFDENGLIKSLPNTPWQLRAAALDRKLTPLEWSAVLLHLIKHRGYLSQRKNEGETADKELGALLKGVAGNA |
| acid | | HALQTGDFRTPAELALNKFEKESGHIRNQRSDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSPELQDEIGTAFSLEKTDEDITGRLKDRIQPEILEALLKHI |
| Nme1Cas9 | | SFDKFVQISLKALRRIVPLMEQGKRYDEACAEIYGDHYGKKNTEEKIYLPPIPADEIRNPVVLRALSQARKVINGVVRRYGSP |
| RuvC | | ARIHIETAREVGKSFKDRKEIEKRQEENRKDREKAAAKFREYFPNFVGEPKSKDILKLRLYEQQHGKCLYSGKEINLGRLNEK |
| nickase | | GYVEIDHALPFSRTWDDSFNNKVLVLGSENQNKGNQTPYEYENGKDNSREWQEFKARVETSRFPRSKKQRILLQKEDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVQGKDEEDWQLIDDSFN |
| | | FKFSLHPNDLVEVITKKARMEGYFASCHRGTGNINIRIHDLDHKIGKNGILEGIGVKTALSFQKYQIDELGKEIRPCRLKKRP |
| | | PVR |
| Exemplary | 235 | GCTGCTTTTAAGCCTAATTCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTACTCGTCGT |
| encoding | | CTTCTTAAGCGTGAGGGTGTTCTTCAGGCTGCTAATTTTGATGAGAATGGTCTTATTAAGTCTCTTCCTAATACTCCTTGGCA |
| Nme1Cas 9 | | GCTTCGTGCTGCTGCTCTTGATCGTAAGCTTACTCCTCTTGAGTGGTCTGCTGTTCTTCTTCATCTTATTAAGCATCGTGGTT |
| RuvC | | ATCTTTCTCAGCGTAAGAATGAGGGTGAGACTGCTGATAAGGAGCTTGGTGCTCTTCTTAAGGGTGTTGCTGGTAATGCTCAT |
| nickase | | GCTCTTCAGACTGGTGATTTTCGTACTCCTGCTGAGCTTGCTCTTAATAAGTTTGAGAAGGAGTCTGGTCATATTCGTAATCA |
| | | GCGTTCTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCTTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTCAGGGTAAGGATGAGGAGGATTGGCAGCTTATTGATGATTCTTTTAATTTT |
| | | AAGTTTTCTCTTCATCCTAATGATCTTGTTGAGGTTATTACTAAGAAGGCTCGTATGTTTGGTTATTTTGCTTCTTGTCATCG |
| | | TGGTACTGGTAATATTAATATTCGTATTCATGATCTTGATCATAAGATTGGTAAGAATGGTATTCTTGAGGGTATTGGTGTTA |
| | | AGACTGCTCTTTCTTTTCAGAAGTATCAGATTGATGAGCTTGGTAAGGAGATTCGTCCTTGTCGTCTTAAGAAGCGTCCTCCT |
| | | GTTCGT |
| Exemplary | 236 | GCCGCCTTCAAGCCCAACTCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGACCCGGCGG |
| encoding | | CTGCTGAAGCGGGAGGGCGTGCTGCAGGCCGCCAACTTCGACGAGAACGGCCTGATCAAGTCCCTGCCCAACACCCCCTGGCA |
| Nme1Cas9 | | GCTGCGGGCCGCCGCCCTGGACCGGAAGCTGACCCCCCTGGAGTGGTCCGCCGTGCTGCTGCACCTGATCAAGCACCGGGGCT |
| Ruvc | | ACCTGTCCCAGCGGAAGAACGAGGGCGAGACCGCCGACAAGGAGCTGGGCGCCCTGCTGAAGGGCGTGGCCGGCAACGCCCAC |
| nickase | | GCCCTGCAGACCGGCGACTTCCGGACCCCCGCCGAGCTGGCCCTGAACAAGTTCGAGAAGGAGTCCGGCCACATCCGGAACCA |
| | | GCGGTCCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCTGG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGCAGGGCAAGGACGAGGAGGACTGGCAGCTGATCGACGACTCCTTCAACTTC |
| | | AAGTTCTCCCTGCACCCCAACGACCTGGTGGAGGTGATCACCAAGAAGGCCCGGATGTTCGGCTACTTCGCCTCCTGCCACCG |
| | | GGGCACCGGCAACATCAACATCCGGATCCACGACCTGGACCACAAGATCGGCAAGAACGGCATCCTGGAGGGCATCGGCGTGA |
| | | AGACCGCCCTGTCCTTCCAGAAGTACCAGATCGACGAGCTGGGCAAGGAGATCCGGCCCTGCCGGCTGAAGAAGCGGCCCCCC |
| | | GTGCGG |
| Exemplary | 237 | GCAGCATTCAAACCAAACTCAATCAACTACATCCTAGGACTAGCAATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAACACGACGA |
| encoding | | CTACTAAAACGAGAAGGAGTACTACAAGCAGCAAACTTCGACGAAAACGGACTAATCAAATCACTACCAAACACACCATGACA |
| Nme1Cas9 | | ACTACGAGCAGCAGCACTAGACCGAAAACTAACACCACTAGAATGATCAGCAGTACTACTACACCTAATCAAACACCGAGGAT |
| RuvC | | ACCTATCACAACGAAAAAACGAAGGAGAAACAGCAGACAAAGAACTAGGAGCACTACTAAAAGGAGTAGCAGGAAACGCACAC |
| nickase | | GCACTACAAACAGGAGACTTCCGAACACCAGCAGAACTAGCACTAAACAAATTCGAAAAAGAATCAGGACACATCCGAAACCA |
| | | ACGATCAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACCACGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACTAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTACAAGGAAAAGACGAAGAAGACTGACAACTAATCGACGACTCATTCAACTTC |
| | | AAATTCTCACTACACCCAAACGACCTAGTAGAAGTAATCACAAAAAAAGCACGAATGTTCGGATACTTCGCATCATGCCACCG |
| | | AGGAACAGGAAACATCAACATCCGAATCCACGACCTAGACCACAAAATCGGAAAAAACGGAATCCTAGAAGGAATCGGAGTAA |
| | | AAACAGCACTATCATTCCAAAAATACCAAATCGACGAACTAGGAAAAGAAATCCGACCATGCCGACTAAAAAAACGACCACCA |
| | | GTACGA |
| Exemplary | 238 | ATGGCTGCTTTTAAGCCTAATTCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTACTCGT |
| frame for | | CGTCTTCTTAAGCGTGAGGGTGTTCTTCAGGCTGCTAATTTTGATGAGAATGGTCTTATTAAGTCTCTTCCTAATACTCCTTG |
| Nme1Cas 9 | | GCAGCTTCGTGCTGCTGCTCTTGATCGTAAGCTTACTCCTCTTGAGTGGTCTGCTGTTCTTCTTCATCTTATTAAGCATCGTG |
| RuvC | | GTTATCTTTCTCAGCGTAAGAATGAGGGTGAGACTGCTGATAAGGAGCTTGGTGCTCTTCTTAAGGGTGTTGCTGGTAATGCT |
| nickase | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTTCTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | TTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTCAGGGTAAGGATGAGGAGGATTGGCAGCTTATTGATGATTCTTTTAAT |
| | | TTTAAGTTTTCTCTTCATCCTAATGATCTTGTTGAGGTTATTACTAAGAAGGCTCGTATGTTTGGTTATTTTGCTTCTTGTCA |
| | | TCGTGGTACTGGTAATATTAATATTCGTATTCATGATCTTGATCATAAGATTGGTAAGAATGGTATTCTTGAGGGTATTGGTG |
| | | TTAAGACTGCTCTTTCTTTTCAGAAGTATCAGATTGATGAGCTTGGTAAGGAGATTCGTCCTTGTCGTCTTAAGAAGCGTCCT |
| | | CCTGTTCGTUGA |
| Exemplary | 239 | ATGGCCGCCTTCAAGCCCAACTCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGACCCGG |
| frame for | | CGGCTGCTGAAGCGGGAGGGCGTGCTGCAGGCCGCCAACTTCGACGAGAACGGCCTGATCAAGTCCCTGCCCAACACCCCCTG |
| Nme1Cas 9 | | GCAGCTGCGGGCCGCCGCCCTGGACCGGAAGCTGACCCCCCTGGAGTGGTCCGCCGTGCTGCTGCACCTGATCAAGCACCGGG |
| Ruvc | | GCTACCTGTCCCAGCGGAAGAACGAGGGCGAGACCGCCGACAAGGAGCTGGGCGCCCTGCTGAAGGGCGTGGCCGGCAACGCC |
| nickase | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGTCCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | TGGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGCAGGGCAAGGACGAGGAGGACTGGCAGCTGATCGACGACTCCTTCAAC |
| | | TTCAAGTTCTCCCTGCACCCCAACGACCTGGTGGAGGTGATCACCAAGAAGGCCCGGATGTTCGGCTACTTCGCCTCCTGCCA |
| | | CCGGGGCACCGGCAACATCAACATCCGGATCCACGACCTGGACCACAAGATCGGCAAGAACGGCATCCTGGAGGGCATCGGCG |
| | | TGAAGACCGCCCTGTCCTTCCAGAAGTACCAGATCGACGAGCTGGGCAAGGAGATCCGGCCCTGCCGGCTGAAGAAGCGGCCC |
| | | CCCGTGCGGUGA |
| Exemplary | 240 | ATGGCAGCATTCAAACCAAACTCAATCAACTACATCCTAGGACTAGCAATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAACACGA |
| frame for | | CGACTACTAAAACGAGAAGGAGTACTACAAGCAGCAAACTTCGACGAAAACGGACTAATCAAATCACTACCAAACACACCATG |
| Nme1Cas9 | | ACAACTACGAGCAGCAGCACTAGACCGAAAACTAACACCACTAGAATGATCAGCAGTACTACTACACCTAATCAAACACCGAG |
| RuvC | | GATACCTATCACAACGAAAAAACGAAGGAGAAACAGCAGACAAAGAACTAGGAGCACTACTAAAAGGAGTAGCAGGAAACGCA |
| nickase | | CACGCACTACAAACAGGAGACTTCCGAACACCAGCAGAACTAGCACTAAACAAATTCGAAAAAGAATCAGGACACATCCGAAA |
| | | CCAACGATCAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACCACGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | TAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTACAAGGAAAAGACGAAGAAGACTGACAACTAATCGACGACTCATTCAAC |
| | | TTCAAATTCTCACTACACCCAAACGACCTAGTAGAAGTAATCACAAAAAAAGCACGAATGTTCGGATACTTCGCATCATGCCA |
| | | CCGAGGAACAGGAAACATCAACATCCGAATCCACGACCTAGACCACAAAATCGGAAAAAACGGAATCCTAGAAGGAATCGGAG |
| | | TAAAAACAGCACTATCATTCCAAAAATACCAAATCGACGAACTAGGAAAAGAAATCCGACCATGCCGACTAAAAAAACGACCA |
| | | CCAGTACGAUAA |
| Exemplary | 241 | MAAFKPNSINYILGLDIGIASVGWAMVEIDEEENPIRLIDLGVRVFERAEVPKTGDSLAMARRLARSVRRLTRRRAHRLLRTR |
| amino | | RLLKREGVLQAANEDENGLIKSLPNTPWQLRAAALDRKLTPLEWSAVLLHLIKHRGYLSQRKNEGETADKELGALLKGVAGNA |
| acid | | HALQTGDFRTPAELALNKFEKESGHIRNQRSDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSPELQDEIGTAFSLEKTDEDITGRLKDRIQPEILEALLKHI |
| Nme1Cas9 | | SFDKFVQISLKALRRIVPLMEQGKRYDEACAEIYGDHYGKKNTEEKIYLPPIPADEIRNPVVLRALSQARKVINGVVRRYGSP |
| HNH | | ARIHIETAREVGKSFKDRKEIEKRQEENRKDREKAAAKFREYFPNFVGEPKSKDILKLRLYEQQHGKCLYSGKEINLGRLNEK |
| nickase | | GYVEIDAALPFSRTWDDSFNNKVLVLGSENQNKGNQTPYEYENGKDNSREWQEFKARVETSRFPRSKKQRILLQKEDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVQGKDEEDWQLIDDSFN |
| | | FKFSLHPNDLVEVITKKARMFGYFASCHRGTGNINIRIHDLDHKIGKNGILEGIGVKTALSFQKYQIDELGKEIRPCRLKKRP |
| | | PVR |
| Exemplary | 242 | GCTGCTTTTAAGCCTAATTCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTACTCGTCGT |
| encoding | | CTTCTTAAGCGTGAGGGTGTTCTTCAGGCTGCTAATTTTGATGAGAATGGTCTTATTAAGTCTCTTCCTAATACTCCTTGGCA |
| Nme1Cas9 | | GCTTCGTGCTGCTGCTCTTGATCGTAAGCTTACTCCTCTTGAGTGGTCTGCTGTTCTTCTTCATCTTATTAAGCATCGTGGTT |
| HNH | | ATCTTTCTCAGCGTAAGAATGAGGGTGAGACTGCTGATAAGGAGCTTGGTGCTCTTCTTAAGGGTGTTGCTGGTAATGCTCAT |
| nickase | | GCTCTTCAGACTGGTGATTTTCGTACTCCTGCTGAGCTTGCTCTTAATAAGTTTGAGAAGGAGTCTGGTCATATTCGTAATCA |
| | | GCGTTCTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCTTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTCAGGGTAAGGATGAGGAGGATTGGCAGCTTATTGATGATTCTTTTAATTTT |
| | | AAGTTTTCTCTTCATCCTAATGATCTTGTTGAGGTTATTACTAAGAAGGCTCGTATGTTTGGTTATTTTGCTTCTTGTCATCG |
| | | TGGTACTGGTAATATTAATATTCGTATTCATGATCTTGATCATAAGATTGGTAAGAATGGTATTCTTGAGGGTATTGGTGTTA |
| | | AGACTGCTCTTTCTTTTCAGAAGTATCAGATTGATGAGCTTGGTAAGGAGATTCGTCCTTGTCGTCTTAAGAAGCGTCCTCCT |
| | | GTTCGT |
| Exemplary | 243 | GCCGCCTTCAAGCCCAACTCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGACCCGGCGG |
| encoding | | CTGCTGAAGCGGGAGGGCGTGCTGCAGGCCGCCAACTTCGACGAGAACGGCCTGATCAAGTCCCTGCCCAACACCCCCTGGCA |
| Nme1Cas9 | | GCTGCGGGCCGCCGCCCTGGACCGGAAGCTGACCCCCCTGGAGTGGTCCGCCGTGCTGCTGCACCTGATCAAGCACCGGGGCT |
| HNH | | ACCTGTCCCAGCGGAAGAACGAGGGCGAGACCGCCGACAAGGAGCTGGGCGCCCTGCTGAAGGGCGTGGCCGGCAACGCCCAC |
| nickase | | GCCCTGCAGACCGGCGACTTCCGGACCCCCGCCGAGCTGGCCCTGAACAAGTTCGAGAAGGAGTCCGGCCACATCCGGAACCA |
| | | GCGGTCCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCTGG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGCAGGGCAAGGACGAGGAGGACTGGCAGCTGATCGACGACTCCTTCAACTTC |
| | | AAGTTCTCCCTGCACCCCAACGACCTGGTGGAGGTGATCACCAAGAAGGCCCGGATGTTCGGCTACTTCGCCTCCTGCCACCG |
| | | GGGCACCGGCAACATCAACATCCGGATCCACGACCTGGACCACAAGATCGGCAAGAACGGCATCCTGGAGGGCATCGGCGTGA |
| | | AGACCGCCCTGTCCTTCCAGAAGTACCAGATCGACGAGCTGGGCAAGGAGATCCGGCCCTGCCGGCTGAAGAAGCGGCCCCCC |
| | | GTGCGG |
| Exemplary | 244 | GCAGCATTCAAACCAAACTCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAACACGACGA |
| encoding | | CTACTAAAACGAGAAGGAGTACTACAAGCAGCAAACTTCGACGAAAACGGACTAATCAAATCACTACCAAACACACCATGACA |
| Nme1Cas9 | | ACTACGAGCAGCAGCACTAGACCGAAAACTAACACCACTAGAATGATCAGCAGTACTACTACACCTAATCAAACACCGAGGAT |
| HNH | | ACCTATCACAACGAAAAAACGAAGGAGAAACAGCAGACAAAGAACTAGGAGCACTACTAAAAGGAGTAGCAGGAAACGCACAC |
| nickase | | GCACTACAAACAGGAGACTTCCGAACACCAGCAGAACTAGCACTAAACAAATTCGAAAAAGAATCAGGACACATCCGAAACCA |
| | | ACGATCAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACGCAGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACTAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTACAAGGAAAAGACGAAGAAGACTGACAACTAATCGACGACTCATTCAACTTC |
| | | AAATTCTCACTACACCCAAACGACCTAGTAGAAGTAATCACAAAAAAAGCACGAATGTTCGGATACTTCGCATCATGCCACCG |
| | | AGGAACAGGAAACATCAACATCCGAATCCACGACCTAGACCACAAAATCGGAAAAAACGGAATCCTAGAAGGAATCGGAGTAA |
| | | AAACAGCACTATCATTCCAAAAATACCAAATCGACGAACTAGGAAAAGAAATCCGACCATGCCGACTAAAAAAACGACCACCA |
| | | GTACGA |
| Exemplary | 245 | ATGGCTGCTTTTAAGCCTAATTCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTACTCGT |
| frame for | | CGTCTTCTTAAGCGTGAGGGTGTTCTTCAGGCTGCTAATTTTGATGAGAATGGTCTTATTAAGTCTCTTCCTAATACTCCTTG |
| Nme1Cas9 | | GCAGCTTCGTGCTGCTGCTCTTGATCGTAAGCTTACTCCTCTTGAGTGGTCTGCTGTTCTTCTTCATCTTATTAAGCATCGTG |
| HNH | | GTTATCTTTCTCAGCGTAAGAATGAGGGTGAGACTGCTGATAAGGAGCTTGGTGCTCTTCTTAAGGGTGTTGCTGGTAATGCT |
| nickase | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTTCTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | TTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTCAGGGTAAGGATGAGGAGGATTGGCAGCTTATTGATGATTCTTTTAAT |
| | | TTTAAGTTTTCTCTTCATCCTAATGATCTTGTTGAGGTTATTACTAAGAAGGCTCGTATGTTTGGTTATTTTGCTTCTTGTCA |
| | | TCGTGGTACTGGTAATATTAATATTCGTATTCATGATCTTGATCATAAGATTGGTAAGAATGGTATTCTTGAGGGTATTGGTG |
| | | TTAAGACTGCTCTTTCTTTTCAGAAGTATCAGATTGATGAGCTTGGTAAGGAGATTCGTCCTTGTCGTCTTAAGAAGCGTCCT |
| | | CCTGTTCGTUGA |
| Exemplary | 246 | ATGGCCGCCTTCAAGCCCAACTCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGACCCGG |
| frame for | | CGGCTGCTGAAGCGGGAGGGCGTGCTGCAGGCCGCCAACTTCGACGAGAACGGCCTGATCAAGTCCCTGCCCAACACCCCCTG |
| Nme1Cas9 | | GCAGCTGCGGGCCGCCGCCCTGGACCGGAAGCTGACCCCCCTGGAGTGGTCCGCCGTGCTGCTGCACCTGATCAAGCACCGGG |
| HNH | | GCTACCTGTCCCAGCGGAAGAACGAGGGCGAGACCGCCGACAAGGAGCTGGGCGCCCTGCTGAAGGGCGTGGCCGGCAACGCC |
| nickase | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGTCCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | TGGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGCAGGGCAAGGACGAGGAGGACTGGCAGCTGATCGACGACTCCTTCAAC |
| | | TTCAAGTTCTCCCTGCACCCCAACGACCTGGTGGAGGTGATCACCAAGAAGGCCCGGATGTTCGGCTACTTCGCCTCCTGCCA |
| | | CCGGGGCACCGGCAACATCAACATCCGGATCCACGACCTGGACCACAAGATCGGCAAGAACGGCATCCTGGAGGGCATCGGCG |
| | | TGAAGACCGCCCTGTCCTTCCAGAAGTACCAGATCGACGAGCTGGGCAAGGAGATCCGGCCCTGCCGGCTGAAGAAGCGGCCC |
| | | CCCGTGCGGUGA |
| Exemplary | 247 | ATGGCAGCATTCAAACCAAACTCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAACACGA |
| frame for | | CGACTACTAAAACGAGAAGGAGTACTACAAGCAGCAAACTTCGACGAAAACGGACTAATCAAATCACTACCAAACACACCATG |
| Nme1Cas9 | | ACAACTACGAGCAGCAGCACTAGACCGAAAACTAACACCACTAGAATGATCAGCAGTACTACTACACCTAATCAAACACCGAG |
| HNH | | GATACCTATCACAACGAAAAAACGAAGGAGAAACAGCAGACAAAGAACTAGGAGCACTACTAAAAGGAGTAGCAGGAAACGCA |
| nickase | | CACGCACTACAAACAGGAGACTTCCGAACACCAGCAGAACTAGCACTAAACAAATTCGAAAAAGAATCAGGACACATCCGAAA |
| | | CCAACGATCAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACGCAGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | TAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTACAAGGAAAAGACGAAGAAGACTGACAACTAATCGACGACTCATTCAAC |
| | | TTCAAATTCTCACTACACCCAAACGACCTAGTAGAAGTAATCACAAAAAAAGCACGAATGTTCGGATACTTCGCATCATGCCA |
| | | CCGAGGAACAGGAAACATCAACATCCGAATCCACGACCTAGACCACAAAATCGGAAAAAACGGAATCCTAGAAGGAATCGGAG |
| | | TAAAAACAGCACTATCATTCCAAAAATACCAAATCGACGAACTAGGAAAAGAAATCCGACCATGCCGACTAAAAAAACGACCA |
| | | CCAGTACGAUAA |
| Exemplary | 248 | MAAFKPNPINYILGLDIGIASVGWAMVEIDEEENPIRLIDLGVRVFERAEVPKTGDSLAMARRLARSVRRLTRRRAHRLLRAR |
| amino | | RLLKREGVLQAADFDENGLIKSLPNTPWQLRAAALDRKLTPLEWSAVLLHLIKHRGYLSQRKNEGETADKELGALLKGVANNA |
| acid | | HALQTGDFRTPAELALNKFEKESGHIRNQRGDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSSELQDEIGTAFSLEKTDEDITGRLKDRVQPEILEALLKHI |
| Nme2Cas9 | | SFDKFVQISLKALRRIVPLMEQGKRYDEACAEIYGDHYGKKNTEEKIYLPPIPADEIRNPVVLRALSQARKVINGVVRRYGSP |
| cleavase | | ARIHIETAREVGKSFKDRKEIEKRQEENRKDREKAAAKFREYFPNFVGEPKSKDILKLRLYEQQHGKCLYSGKEINLVRLNEK |
| | | GYVEIDHALPFSRTWDDSFNNKVLVLGSFNQNKGNQTPYEYENGKDNSREWQEFKARVETSRFPRSKKQRILLQKFDEDGEKE |
| | | CNLNDTRYVNRFLCQFVADHILLTGKGKRRVFASNGQITNLLRGFWGLRKVRAENDRHHALDAVVVACSTVAMQQKITREVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Exemplary | 249 | GCTGCTTTTAAGCCTAATCCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme2Cas9 | | GCTTCGTGCTGCTGCTCTTGATCGTAAGCTTACTCCTCTTGAGTGGTCTGCTGTTCTTCTTCATCTTATTAAGCATCGTGGTT |
| cleavase | | ATCTTTCTCAGCGTAAGAATGAGGGTGAGACTGCTGATAAGGAGCTTGGTGCTCTTCTTAAGGGTGTTGCTAATAATGCTCAT |
| | | GCTCTTCAGACTGGTGATTTTCGTACTCCTGCTGAGCTTGCTCTTAATAAGTTTGAGAAGGAGTCTGGTCATATTCGTAATCA |
| | | GCGTGGTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTTCTGAGCTTCAGGATGAGATTGGTACTGCTTTTTCTCTTT |
| | | TTAAGACTGATGAGGATATTACTGGTCGTCTTAAGGATCGTGTTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGTTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGTGT |
| | | AATCTTAATGATACTCGTTATGTTAATCGTTTTCTTTGTCAGTTTGTTGCTGATCATATTCTTCTTACTGGTAAGGGTAAGCG |
| | | TCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTAAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTGCTCATAAGGATACTCTTCGTTCTGCTAAGCGTTTTGTTAAGCATAATGAGAAGAT |
| | | TTCTGTTAAGCGTGTTTGGCTTACTGAGATTAAGCTTGCTGATCTTGAGAATATGGTTAATTATAAGAATGGTCGTGAGATTG |
| | | AGCTTTATGAGGCTCTTAAGGCTCGTCTTGAGGCTTATGGTGGTAATGCTAAGCAGGCTTTTGATCCTAAGGATAATCCTTTT |
| | | TATAAGAAGGGTGGTCAGCTTGTTAAGGCTGTTCGTGTTGAGAAGACTCAGGAGTCTGGTGTTCTTCTTAATAAGAAGAATGC |
| | | TTATACTATTGCTGATAATGGTGATATGGTTCGTGTTGATGTTTTTTGTAAGGTTGATAAGAAGGGTAAGAATCAGTATTTTA |
| | | TTGTTCCTATTTATGCTTGGCAGGTTGCTGAGAATATTCTTCCTGATATTGATTGTAAGGGTTATCGTATTGATGATTCTTAT |
| | | ACTTTTTGTTTTTCTCTTCATAAGTATGATCTTATTGCTTTTCAGAAGGATGAGAAGTCTAAGGTTGAGTTTGCTTATTATAT |
| | | TAATTGTGATTCTTCTAATGGTCGTTTTTATCTTGCTTGGCATGATAAGGGTTCTAAGGAGCAGCAGTTTCGTATTTCTACTC |
| | | AGAATCTTGTTCTTATTCAGAAGTATCAGGTTAATGAGCTTGGTAAGGAGATTCGTCCTTGTCGTCTTAAGAAGCGTCCTCCT |
| | | GTTCGT |
| Exemplary | 250 | GCCGCCTTCAAGCCCAACCCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| cleavase | | ACCTGTCCCAGCGGAAGAACGAGGGCGAGACCGCCGACAAGGAGCTGGGCGCCCTGCTGAAGGGCGTGGCCAACAACGCCCAC |
| | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 251 | GCAGCATTCAAACCAAACCCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme2Cas9 | | ACTACGAGCAGCAGCACTAGACCGAAAACTAACACCACTAGAATGATCAGCAGTACTACTACACCTAATCAAACACCGAGGAT |
| cleavase | | ACCTATCACAACGAAAAAACGAAGGAGAAACAGCAGACAAAGAACTAGGAGCACTACTAAAAGGAGTAGCAAACAACGCACAC |
| | | GCACTACAAACAGGAGACTTCCGAACACCAGCAGAACTAGCACTAAACAAATTCGAAAAAGAATCAGGACACATCCGAAACCA |
| | | ACGAGGAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCATCAGAACTACAAGACGAAATCGGAACAGCATTCTCACTAT |
| | | TCAAAACAGACGAAGACATCACAGGACGACTAAAAGACCGAGTACAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGTACGACTAAACGAAAAAGGA |
| | | TACGTAGAAATCGACCACGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAATGC |
| | | AACCTAAACGACACACGATACGTAAACCGATTCCTATGCCAATTCGTAGCAGACCACATCCTACTAACAGGAAAAGGAAAACG |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAAAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGAGCACACAAAGACACACTACGATCAGCAAAACGATTCGTAAAACACAACGAAAAAAT |
| | | CTCAGTAAAACGAGTATGACTAACAGAAATCAAACTAGCAGACCTAGAAAACATGGTAAACTACAAAAACGGACGAGAAATCG |
| | | AACTATACGAAGCACTAAAAGCACGACTAGAAGCATACGGAGGAAACGCAAAACAAGCATTCGACCCAAAAGACAACCCATTC |
| | | TACAAAAAAGGAGGACAACTAGTAAAAGCAGTACGAGTAGAAAAAACACAAGAATCAGGAGTACTACTAAACAAAAAAAACGC |
| | | ATACACAATCGCAGACAACGGAGACATGGTACGAGTAGACGTATTCTGCAAAGTAGACAAAAAAGGAAAAAACCAATACTTCA |
| | | TCGTACCAATCTACGCATGACAAGTAGCAGAAAACATCCTACCAGACATCGACTGCAAAGGATACCGAATCGACGACTCATAC |
| | | ACATTCTGCTTCTCACTACACAAATACGACCTAATCGCATTCCAAAAAGACGAAAAATCAAAAGTAGAATTCGCATACTACAT |
| | | CAACTGCGACTCATCAAACGGACGATTCTACCTAGCATGACACGACAAAGGATCAAAAGAACAACAATTCCGAATCTCAACAC |
| | | AAAACCTAGTACTAATCCAAAAATACCAAGTAAACGAACTAGGAAAAGAAATCCGACCATGCCGACTAAAAAAACGACCACCA |
| | | GTACGA |
| Exemplary | 252 | ATGGCTGCTTTTAAGCCTAATCCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme2Cas9 | | GCAGCTTCGTGCTGCTGCTCTTGATCGTAAGCTTACTCCTCTTGAGTGGTCTGCTGTTCTTCTTCATCTTATTAAGCATCGTG |
| cleavase | | GTTATCTTTCTCAGCGTAAGAATGAGGGTGAGACTGCTGATAAGGAGCTTGGTGCTCTTCTTAAGGGTGTTGCTAATAATGCT |
| | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTGGTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTTCTGAGCTTCAGGATGAGATTGGTACTGCTTTTTCTC |
| | | TTTTTAAGACTGATGAGGATATTACTGGTCGTCTTAAGGATCGTGTTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGTTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | TGTAATCTTAATGATACTCGTTATGTTAATCGTTTTCTTTGTCAGTTTGTTGCTGATCATATTCTTCTTACTGGTAAGGGTAA |
| | | GCGTCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTAAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTGCTCATAAGGATACTCTTCGTTCTGCTAAGCGTTTTGTTAAGCATAATGAGAA |
| | | GATTTCTGTTAAGCGTGTTTGGCTTACTGAGATTAAGCTTGCTGATCTTGAGAATATGGTTAATTATAAGAATGGTCGTGAGA |
| | | TTGAGCTTTATGAGGCTCTTAAGGCTCGTCTTGAGGCTTATGGTGGTAATGCTAAGCAGGCTTTTGATCCTAAGGATAATCCT |
| | | TTTTATAAGAAGGGTGGTCAGCTTGTTAAGGCTGTTCGTGTTGAGAAGACTCAGGAGTCTGGTGTTCTTCTTAATAAGAAGAA |
| | | TGCTTATACTATTGCTGATAATGGTGATATGGTTCGTGTTGATGTTTTTTGTAAGGTTGATAAGAAGGGTAAGAATCAGTATT |
| | | TTATTGTTCCTATTTATGCTTGGCAGGTTGCTGAGAATATTCTTCCTGATATTGATTGTAAGGGTTATCGTATTGATGATTCT |
| | | TATACTTTTTGTTTTTCTCTTCATAAGTATGATCTTATTGCTTTTCAGAAGGATGAGAAGTCTAAGGTTGAGTTTGCTTATTA |
| | | TATTAATTGTGATTCTTCTAATGGTCGTTTTTATCTTGCTTGGCATGATAAGGGTTCTAAGGAGCAGCAGTTTCGTATTTCTA |
| | | CTCAGAATCTTGTTCTTATTCAGAAGTATCAGGTTAATGAGCTTGGTAAGGAGATTCGTCCTTGTCGTCTTAAGAAGCGTCCT |
| | | CCTGTTCGTUGA |
| Exemplary | 253 | ATGGCCGCCTTCAAGCCCAACCCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| cleavase | | GCTACCTGTCCCAGCGGAAGAACGAGGGCGAGACCGCCGACAAGGAGCTGGGCGCCCTGCTGAAGGGCGTGGCCAACAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUGA |
| Exemplary | 254 | ATGGCAGCATTCAAACCAAACCCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme2Cas9 | | ACAACTACGAGCAGCAGCACTAGACCGAAAACTAACACCACTAGAATGATCAGCAGTACTACTACACCTAATCAAACACCGAG |
| cleavase | | GATACCTATCACAACGAAAAAACGAAGGAGAAACAGCAGACAAAGAACTAGGAGCACTACTAAAAGGAGTAGCAAACAACGCA |
| | | CACGCACTACAAACAGGAGACTTCCGAACACCAGCAGAACTAGCACTAAACAAATTCGAAAAAGAATCAGGACACATCCGAAA |
| | | CCAACGAGGAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCATCAGAACTACAAGACGAAATCGGAACAGCATTCTCAC |
| | | TATTCAAAACAGACGAAGACATCACAGGACGACTAAAAGACCGAGTACAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGTACGACTAAACGAAAAA |
| | | GGATACGTAGAAATCGACCACGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | TGCAACCTAAACGACACACGATACGTAAACCGATTCCTATGCCAATTCGTAGCAGACCACATCCTACTAACAGGAAAAGGAAA |
| | | ACGACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAAAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGAGCACACAAAGACACACTACGATCAGCAAAACGATTCGTAAAACACAACGAAAA |
| | | AATCTCAGTAAAACGAGTATGACTAACAGAAATCAAACTAGCAGACCTAGAAAACATGGTAAACTACAAAAACGGACGAGAAA |
| | | TCGAACTATACGAAGCACTAAAAGCACGACTAGAAGCATACGGAGGAAACGCAAAACAAGCATTCGACCCAAAAGACAACCCA |
| | | TTCTACAAAAAAGGAGGACAACTAGTAAAAGCAGTACGAGTAGAAAAAACACAAGAATCAGGAGTACTACTAAACAAAAAAAA |
| | | CGCATACACAATCGCAGACAACGGAGACATGGTACGAGTAGACGTATTCTGCAAAGTAGACAAAAAAGGAAAAAACCAATACT |
| | | TCATCGTACCAATCTACGCATGACAAGTAGCAGAAAACATCCTACCAGACATCGACTGCAAAGGATACCGAATCGACGACTCA |
| | | TACACATTCTGCTTCTCACTACACAAATACGACCTAATCGCATTCCAAAAAGACGAAAAATCAAAAGTAGAATTCGCATACTA |
| | | CATCAACTGCGACTCATCAAACGGACGATTCTACCTAGCATGACACGACAAAGGATCAAAAGAACAACAATTCCGAATCTCAA |
| | | CACAAAACCTAGTACTAATCCAAAAATACCAAGTAAACGAACTAGGAAAAGAAATCCGACCATGCCGACTAAAAAAACGACCA |
| | | CCAGTACGAUAA |
| Exemplary | 255 | MAAFKPNPINYILGLAIGIASVGWAMVEIDEEENPIRLIDLGVRVFERAEVPKTGDSLAMARRLARSVRRLTRRRAHRLLRAR |
| amino | | RLLKREGVLQAADEDENGLIKSLPNTPWQLRAAALDRKLTPLEWSAVLLHLIKHRGYLSQRKNEGETADKELGALLKGVANNA |
| acid | | HALQTGDFRTPAELALNKFEKESGHIRNQRGDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSSELQDEIGTAFSLEKTDEDITGRLKDRVQPEILEALLKHI |
| Nme2Cas9 | | SFDKFVQISLKALRRIVPLMEQGKRYDEACAEIYGDHYGKKNTEEKIYLPPIPADEIRNPVVLRALSQARKVINGVVRRYGSP |
| dCas9 | | ARIHIETAREVGKSFKDRKEIEKRQEENRKDREKAAAKFREYFPNFVGEPKSKDILKLRLYEQQHGKCLYSGKEINLVRLNEK |
| | | GYVEIDAALPESRTWDDSFNNKVLVLGSENQNKGNQTPYEYENGKDNSREWQEFKARVETSRFPRSKKQRILLQKFDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Exemplary | 256 | GCTGCTTTTAAGCCTAATCCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme2Cas9 | | GCTTCGTGCTGCTGCTCTTGATCGTAAGCTTACTCCTCTTGAGTGGTCTGCTGTTCTTCTTCATCTTATTAAGCATCGTGGTT |
| dCas9 | | ATCTTTCTCAGCGTAAGAATGAGGGTGAGACTGCTGATAAGGAGCTTGGTGCTCTTCTTAAGGGTGTTGCTAATAATGCTCAT |
| | | GCTCTTCAGACTGGTGATTTTCGTACTCCTGCTGAGCTTGCTCTTAATAAGTTTGAGAAGGAGTCTGGTCATATTCGTAATCA |
| | | GCGTGGTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTTCTGAGCTTCAGGATGAGATTGGTACTGCTTTTTCTCTTT |
| | | TTAAGACTGATGAGGATATTACTGGTCGTCTTAAGGATCGTGTTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGTTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGTGT |
| | | AATCTTAATGATACTCGTTATGTTAATCGTTTTCTTTGTCAGTTTGTTGCTGATCATATTCTTCTTACTGGTAAGGGTAAGCG |
| | | TCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTAAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTGCTCATAAGGATACTCTTCGTTCTGCTAAGCGTTTTGTTAAGCATAATGAGAAGAT |
| | | TTCTGTTAAGCGTGTTTGGCTTACTGAGATTAAGCTTGCTGATCTTGAGAATATGGTTAATTATAAGAATGGTCGTGAGATTG |
| | | AGCTTTATGAGGCTCTTAAGGCTCGTCTTGAGGCTTATGGTGGTAATGCTAAGCAGGCTTTTGATCCTAAGGATAATCCTTTT |
| | | TATAAGAAGGGTGGTCAGCTTGTTAAGGCTGTTCGTGTTGAGAAGACTCAGGAGTCTGGTGTTCTTCTTAATAAGAAGAATGC |
| | | TTATACTATTGCTGATAATGGTGATATGGTTCGTGTTGATGTTTTTTGTAAGGTTGATAAGAAGGGTAAGAATCAGTATTTTA |
| | | TTGTTCCTATTTATGCTTGGCAGGTTGCTGAGAATATTCTTCCTGATATTGATTGTAAGGGTTATCGTATTGATGATTCTTAT |
| | | ACTTTTTGTTTTTCTCTTCATAAGTATGATCTTATTGCTTTTCAGAAGGATGAGAAGTCTAAGGTTGAGTTTGCTTATTATAT |
| | | TAATTGTGATTCTTCTAATGGTCGTTTTTATCTTGCTTGGCATGATAAGGGTTCTAAGGAGCAGCAGTTTCGTATTTCTACTC |
| | | AGAATCTTGTTCTTATTCAGAAGTATCAGGTTAATGAGCTTGGTAAGGAGATTCGTCCTTGTCGTCTTAAGAAGCGTCCTCCT |
| | | GTTCGT |
| Exemplary | 257 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| dCas9 | | ACCTGTCCCAGCGGAAGAACGAGGGCGAGACCGCCGACAAGGAGCTGGGCGCCCTGCTGAAGGGCGTGGCCAACAACGCCCAC |
| | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 258 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| dCas9 | | ACCTGTCCCAGCGGAAGAACGAGGGCGAGACCGCCGACAAGGAGCTGGGCGCCCTGCTGAAGGGCGTGGCCAACAACGCCCAC |
| | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 259 | ATGGCTGCTTTTAAGCCTAATCCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme2Cas9 | | GCAGCTTCGTGCTGCTGCTCTTGATCGTAAGCTTACTCCTCTTGAGTGGTCTGCTGTTCTTCTTCATCTTATTAAGCATCGTG |
| dCas9 | | GTTATCTTTCTCAGCGTAAGAATGAGGGTGAGACTGCTGATAAGGAGCTTGGTGCTCTTCTTAAGGGTGTTGCTAATAATGCT |
| | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTGGTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTTCTGAGCTTCAGGATGAGATTGGTACTGCTTTTTCTC |
| | | TTTTTAAGACTGATGAGGATATTACTGGTCGTCTTAAGGATCGTGTTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGTTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | TGTAATCTTAATGATACTCGTTATGTTAATCGTTTTCTTTGTCAGTTTGTTGCTGATCATATTCTTCTTACTGGTAAGGGTAA |
| | | GCGTCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTAAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTGCTCATAAGGATACTCTTCGTTCTGCTAAGCGTTTTGTTAAGCATAATGAGAA |
| | | GATTTCTGTTAAGCGTGTTTGGCTTACTGAGATTAAGCTTGCTGATCTTGAGAATATGGTTAATTATAAGAATGGTCGTGAGA |
| | | TTGAGCTTTATGAGGCTCTTAAGGCTCGTCTTGAGGCTTATGGTGGTAATGCTAAGCAGGCTTTTGATCCTAAGGATAATCCT |
| | | TTTTATAAGAAGGGTGGTCAGCTTGTTAAGGCTGTTCGTGTTGAGAAGACTCAGGAGTCTGGTGTTCTTCTTAATAAGAAGAA |
| | | TGCTTATACTATTGCTGATAATGGTGATATGGTTCGTGTTGATGTTTTTTGTAAGGTTGATAAGAAGGGTAAGAATCAGTATT |
| | | TTATTGTTCCTATTTATGCTTGGCAGGTTGCTGAGAATATTCTTCCTGATATTGATTGTAAGGGTTATCGTATTGATGATTCT |
| | | TATACTTTTTGTTTTTCTCTTCATAAGTATGATCTTATTGCTTTTCAGAAGGATGAGAAGTCTAAGGTTGAGTTTGCTTATTA |
| | | TATTAATTGTGATTCTTCTAATGGTCGTTTTTATCTTGCTTGGCATGATAAGGGTTCTAAGGAGCAGCAGTTTCGTATTTCTA |
| | | CTCAGAATCTTGTTCTTATTCAGAAGTATCAGGTTAATGAGCTTGGTAAGGAGATTCGTCCTTGTCGTCTTAAGAAGCGTCCT |
| | | CCTGTTCGTUGA |
| Exemplary | 260 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| dCas9 | | GCTACCTGTCCCAGCGGAAGAACGAGGGCGAGACCGCCGACAAGGAGCTGGGCGCCCTGCTGAAGGGCGTGGCCAACAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUGA |
| Exemplary | 261 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| dCas9 | | GCTACCTGTCCCAGCGGAAGAACGAGGGCGAGACCGCCGACAAGGAGCTGGGCGCCCTGCTGAAGGGCGTGGCCAACAACGCC |
| | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUAA |
| Exemplary | 262 | MAAFKPNPINYILGLAIGIASVGWAMVEIDEEENPIRLIDLGVRVFERAEVPKTGDSLAMARRLARSVRRLTRRRAHRLLRAR |
| amino | | RLLKREGVLQAADFDENGLIKSLPNTPWQLRAAALDRKLTPLEWSAVLLHLIKHRGYLSQRKNEGETADKELGALLKGVANNA |
| acid | | HALQTGDFRTPAELALNKFEKESGHIRNQRGDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSSELQDEIGTAFSLFKTDEDITGRLKDRVQPEILEALLKHI |
| Nme2Cas9 | | SFDKFVQISLKALRRIVPLMEQGKRYDEACAEIYGDHYGKKNTEEKIYLPPIPADEIRNPVVLRALSQARKVINGVVRRYGSP |
| Ruvc | | ARIHIETAREVGKSFKDRKEIEKRQEENRKDREKAAAKFREYFPNFVGEPKSKDILKLRLYEQQHGKCLYSGKEINLVRLNEK |
| nickase | | GYVEIDHALPFSRTWDDSFNNKVLVLGSENQNKGNQTPYEYENGKDNSREWQEFKARVETSRFPRSKKQRILLQKEDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Exemplary | 263 | GCTGCTTTTAAGCCTAATCCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme2Cas9 | | GCTTCGTGCTGCTGCTCTTGATCGTAAGCTTACTCCTCTTGAGTGGTCTGCTGTTCTTCTTCATCTTATTAAGCATCGTGGTT |
| Ruvc | | ATCTTTCTCAGCGTAAGAATGAGGGTGAGACTGCTGATAAGGAGCTTGGTGCTCTTCTTAAGGGTGTTGCTAATAATGCTCAT |
| nickase | | GCTCTTCAGACTGGTGATTTTCGTACTCCTGCTGAGCTTGCTCTTAATAAGTTTGAGAAGGAGTCTGGTCATATTCGTAATCA |
| | | GCGTGGTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTTCTGAGCTTCAGGATGAGATTGGTACTGCTTTTTCTCTTT |
| | | TTAAGACTGATGAGGATATTACTGGTCGTCTTAAGGATCGTGTTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGTTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGTGT |
| | | AATCTTAATGATACTCGTTATGTTAATCGTTTTCTTTGTCAGTTTGTTGCTGATCATATTCTTCTTACTGGTAAGGGTAAGCG |
| | | TCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTAAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTGCTCATAAGGATACTCTTCGTTCTGCTAAGCGTTTTGTTAAGCATAATGAGAAGAT |
| | | TTCTGTTAAGCGTGTTTGGCTTACTGAGATTAAGCTTGCTGATCTTGAGAATATGGTTAATTATAAGAATGGTCGTGAGATTG |
| | | AGCTTTATGAGGCTCTTAAGGCTCGTCTTGAGGCTTATGGTGGTAATGCTAAGCAGGCTTTTGATCCTAAGGATAATCCTTTT |
| | | TATAAGAAGGGTGGTCAGCTTGTTAAGGCTGTTCGTGTTGAGAAGACTCAGGAGTCTGGTGTTCTTCTTAATAAGAAGAATGC |
| | | TTATACTATTGCTGATAATGGTGATATGGTTCGTGTTGATGTTTTTTGTAAGGTTGATAAGAAGGGTAAGAATCAGTATTTTA |
| | | TTGTTCCTATTTATGCTTGGCAGGTTGCTGAGAATATTCTTCCTGATATTGATTGTAAGGGTTATCGTATTGATGATTCTTAT |
| | | ACTTTTTGTTTTTCTCTTCATAAGTATGATCTTATTGCTTTTCAGAAGGATGAGAAGTCTAAGGTTGAGTTTGCTTATTATAT |
| | | TAATTGTGATTCTTCTAATGGTCGTTTTTATCTTGCTTGGCATGATAAGGGTTCTAAGGAGCAGCAGTTTCGTATTTCTACTC |
| | | AGAATCTTGTTCTTATTCAGAAGTATCAGGTTAATGAGCTTGGTAAGGAGATTCGTCCTTGTCGTCTTAAGAAGCGTCCTCCT |
| | | GTTCGT |
| Exemplary | 264 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| Ruvc | | ACCTGTCCCAGCGGAAGAACGAGGGCGAGACCGCCGACAAGGAGCTGGGCGCCCTGCTGAAGGGCGTGGCCAACAACGCCCAC |
| nickase | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 265 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| Ruvc | | ACCTGTCCCAGCGGAAGAACGAGGGCGAGACCGCCGACAAGGAGCTGGGCGCCCTGCTGAAGGGCGTGGCCAACAACGCCCAC |
| nickase | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 266 | ATGGCTGCTTTTAAGCCTAATCCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme2Cas9 | | GCAGCTTCGTGCTGCTGCTCTTGATCGTAAGCTTACTCCTCTTGAGTGGTCTGCTGTTCTTCTTCATCTTATTAAGCATCGTG |
| Ruvc | | GTTATCTTTCTCAGCGTAAGAATGAGGGTGAGACTGCTGATAAGGAGCTTGGTGCTCTTCTTAAGGGTGTTGCTAATAATGCT |
| nickase | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTGGTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTTCTGAGCTTCAGGATGAGATTGGTACTGCTTTTTCTC |
| | | TTTTTAAGACTGATGAGGATATTACTGGTCGTCTTAAGGATCGTGTTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGTTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | TGTAATCTTAATGATACTCGTTATGTTAATCGTTTTCTTTGTCAGTTTGTTGCTGATCATATTCTTCTTACTGGTAAGGGTAA |
| | | GCGTCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTAAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTGCTCATAAGGATACTCTTCGTTCTGCTAAGCGTTTTGTTAAGCATAATGAGAA |
| | | GATTTCTGTTAAGCGTGTTTGGCTTACTGAGATTAAGCTTGCTGATCTTGAGAATATGGTTAATTATAAGAATGGTCGTGAGA |
| | | TTGAGCTTTATGAGGCTCTTAAGGCTCGTCTTGAGGCTTATGGTGGTAATGCTAAGCAGGCTTTTGATCCTAAGGATAATCCT |
| | | TTTTATAAGAAGGGTGGTCAGCTTGTTAAGGCTGTTCGTGTTGAGAAGACTCAGGAGTCTGGTGTTCTTCTTAATAAGAAGAA |
| | | TGCTTATACTATTGCTGATAATGGTGATATGGTTCGTGTTGATGTTTTTTGTAAGGTTGATAAGAAGGGTAAGAATCAGTATT |
| | | TTATTGTTCCTATTTATGCTTGGCAGGTTGCTGAGAATATTCTTCCTGATATTGATTGTAAGGGTTATCGTATTGATGATTCT |
| | | TATACTTTTTGTTTTTCTCTTCATAAGTATGATCTTATTGCTTTTCAGAAGGATGAGAAGTCTAAGGTTGAGTTTGCTTATTA |
| | | TATTAATTGTGATTCTTCTAATGGTCGTTTTTATCTTGCTTGGCATGATAAGGGTTCTAAGGAGCAGCAGTTTCGTATTTCTA |
| | | CTCAGAATCTTGTTCTTATTCAGAAGTATCAGGTTAATGAGCTTGGTAAGGAGATTCGTCCTTGTCGTCTTAAGAAGCGTCCT |
| | | CCTGTTCGTUGA |
| Exemplary | 267 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| RuvC | | GCTACCTGTCCCAGCGGAAGAACGAGGGCGAGACCGCCGACAAGGAGCTGGGCGCCCTGCTGAAGGGCGTGGCCAACAACGCC |
| nickase | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUGA |
| Exemplary | 268 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| RuvC | | GCTACCTGTCCCAGCGGAAGAACGAGGGCGAGACCGCCGACAAGGAGCTGGGCGCCCTGCTGAAGGGCGTGGCCAACAACGCC |
| nickase | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUAA |
| Exemplary | 269 | MAAFKPNPINYILGLDIGIASVGWAMVEIDEEENPIRLIDLGVRVFERAEVPKTGDSLAMARRLARSVRRLTRRRAHRLLRAR |
| amino | | RLLKREGVLQAADFDENGLIKSLPNTPWQLRAAALDRKLTPLEWSAVLLHLIKHRGYLSQRKNEGETADKELGALLKGVANNA |
| acid | | HALQTGDFRTPAELALNKFEKESGHIRNQRGDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| of | | KGLRYGKDNAEASTLMEMKAYHAISRALEKEGLKDKKSPLNLSSELQDEIGTAFSLEKTDEDITGRLKDRVQPEILEALLKHI |
| Nme2Cas9 | | SFDKFVQISLKALRRIVPLMEQGKRYDEACAEIYGDHYGKKNTEEKIYLPPIPADEIRNPVVLRALSQARKVINGVVRRYGSP |
| HNH | | ARIHIETAREVGKSFKDRKEIEKRQEENRKDREKAAAKFREYFPNFVGEPKSKDILKLRLYEQQHGKCLYSGKEINLVRLNEK |
| nickase | | GYVEIDAALPESRTWDDSFNNKVLVLGSENQNKGNQTPYEYENGKDNSREWQEFKARVETSRFPRSKKQRILLQKFDEDGEKE |
| | | CNLNDTRYVNRFLCQFVADHILLTGKGKRRVFASNGQITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGKPEFEEADTPEKLRTLLAEKLSSRPEAVHEYVTPLFV |
| | | SRAPNRKMSGAHKDTLRSAKRFVKHNEKISVKRVWLTEIKLADLENMVNYKNGREIELYEALKARLEAYGGNAKQAFDPKDNP |
| | | FYKKGGQLVKAVRVEKTQESGVLLNKKNAYTIADNGDMVRVDVFCKVDKKGKNQYFIVPIYAWQVAENILPDIDCKGYRIDDS |
| | | YTFCFSLHKYDLIAFQKDEKSKVEFAYYINCDSSNGRFYLAWHDKGSKEQQFRISTQNLVLIQKYQVNELGKEIRPCRLKKRP |
| | | PVR |
| Exemplary | 270 | GCTGCTTTTAAGCCTAATCCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme2Cas9 | | GCTTCGTGCTGCTGCTCTTGATCGTAAGCTTACTCCTCTTGAGTGGTCTGCTGTTCTTCTTCATCTTATTAAGCATCGTGGTT |
| HNH | | ATCTTTCTCAGCGTAAGAATGAGGGTGAGACTGCTGATAAGGAGCTTGGTGCTCTTCTTAAGGGTGTTGCTAATAATGCTCAT |
| nickase | | GCTCTTCAGACTGGTGATTTTCGTACTCCTGCTGAGCTTGCTCTTAATAAGTTTGAGAAGGAGTCTGGTCATATTCGTAATCA |
| | | GCGTGGTGATTATTCTCATACTTTTTCTCGTAAGGATCTTCAGGCTGAGCTTATTCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTGGTCTTGAGGATACTGCTTTTTTTAAG |
| | | GGTCTTCGTTATGGTAAGGATAATGCTGAGGCTTCTACTCTTATGGAGATGAAGGCTTATCATGCTATTTCTCGTGCTCTTGA |
| | | GAAGGAGGGTCTTAAGGATAAGAAGTCTCCTCTTAATCTTTCTTCTGAGCTTCAGGATGAGATTGGTACTGCTTTTTCTCTTT |
| | | TTAAGACTGATGAGGATATTACTGGTCGTCTTAAGGATCGTGTTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGTTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGTGT |
| | | AATCTTAATGATACTCGTTATGTTAATCGTTTTCTTTGTCAGTTTGTTGCTGATCATATTCTTCTTACTGGTAAGGGTAAGCG |
| | | TCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTAAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTGCTCATAAGGATACTCTTCGTTCTGCTAAGCGTTTTGTTAAGCATAATGAGAAGAT |
| | | TTCTGTTAAGCGTGTTTGGCTTACTGAGATTAAGCTTGCTGATCTTGAGAATATGGTTAATTATAAGAATGGTCGTGAGATTG |
| | | AGCTTTATGAGGCTCTTAAGGCTCGTCTTGAGGCTTATGGTGGTAATGCTAAGCAGGCTTTTGATCCTAAGGATAATCCTTTT |
| | | TATAAGAAGGGTGGTCAGCTTGTTAAGGCTGTTCGTGTTGAGAAGACTCAGGAGTCTGGTGTTCTTCTTAATAAGAAGAATGC |
| | | TTATACTATTGCTGATAATGGTGATATGGTTCGTGTTGATGTTTTTTGTAAGGTTGATAAGAAGGGTAAGAATCAGTATTTTA |
| | | TTGTTCCTATTTATGCTTGGCAGGTTGCTGAGAATATTCTTCCTGATATTGATTGTAAGGGTTATCGTATTGATGATTCTTAT |
| | | ACTTTTTGTTTTTCTCTTCATAAGTATGATCTTATTGCTTTTCAGAAGGATGAGAAGTCTAAGGTTGAGTTTGCTTATTATAT |
| | | TAATTGTGATTCTTCTAATGGTCGTTTTTATCTTGCTTGGCATGATAAGGGTTCTAAGGAGCAGCAGTTTCGTATTTCTACTC |
| | | AGAATCTTGTTCTTATTCAGAAGTATCAGGTTAATGAGCTTGGTAAGGAGATTCGTCCTTGTCGTCTTAAGAAGCGTCCTCCT |
| | | GTTCGT |
| Exemplary | 271 | GCCGCCTTCAAGCCCAACCCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme2Cas9 | | GCTGCGGGCCGCCGCCCTGGACCGGAAGCTGACCCCCCTGGAGTGGTCCGCCGTGCTGCTGCACCTGATCAAGCACCGGGGCT |
| HNH | | ACCTGTCCCAGCGGAAGAACGAGGGCGAGACCGCCGACAAGGAGCTGGGCGCCCTGCTGAAGGGCGTGGCCAACAACGCCCAC |
| nickase | | GCCCTGCAGACCGGCGACTTCCGGACCCCCGCCGAGCTGGCCCTGAACAAGTTCGAGAAGGAGTCCGGCCACATCCGGAACCA |
| | | GCGGGGCGACTACTCCCACACCTTCTCCCGGAAGGACCTGCAGGCCGAGCTGATCCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGCCATCTCCCGGGCCCTGGA |
| | | GAAGGAGGGCCTGAAGGACAAGAAGTCCCCCCTGAACCTGTCCTCCGAGCTGCAGGACGAGATCGGCACCGCCTTCTCCCTGT |
| | | TCAAGACCGACGAGGACATCACCGGCCGGCTGAAGGACCGGGTGCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGTGCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGTGC |
| | | AACCTGAACGACACCCGGTACGTGAACCGGTTCCTGTGCCAGTTCGTGGCCGACCACATCCTGCTGACCGGCAAGGGCAAGCG |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCAAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCGCCCACAAGGACACCCTGCGGTCCGCCAAGCGGTTCGTGAAGCACAACGAGAAGAT |
| | | CTCCGTGAAGCGGGTGTGGCTGACCGAGATCAAGCTGGCCGACCTGGAGAACATGGTGAACTACAAGAACGGCCGGGAGATCG |
| | | AGCTGTACGAGGCCCTGAAGGCCCGGCTGGAGGCCTACGGCGGCAACGCCAAGCAGGCCTTCGACCCCAAGGACAACCCCTTC |
| | | TACAAGAAGGGCGGCCAGCTGGTGAAGGCCGTGCGGGTGGAGAAGACCCAGGAGTCCGGCGTGCTGCTGAACAAGAAGAACGC |
| | | CTACACCATCGCCGACAACGGCGACATGGTGCGGGTGGACGTGTTCTGCAAGGTGGACAAGAAGGGCAAGAACCAGTACTTCA |
| | | TCGTGCCCATCTACGCCTGGCAGGTGGCCGAGAACATCCTGCCCGACATCGACTGCAAGGGCTACCGGATCGACGACTCCTAC |
| | | ACCTTCTGCTTCTCCCTGCACAAGTACGACCTGATCGCCTTCCAGAAGGACGAGAAGTCCAAGGTGGAGTTCGCCTACTACAT |
| | | CAACTGCGACTCCTCCAACGGCCGGTTCTACCTGGCCTGGCACGACAAGGGCTCCAAGGAGCAGCAGTTCCGGATCTCCACCC |
| | | AGAACCTGGTGCTGATCCAGAAGTACCAGGTGAACGAGCTGGGCAAGGAGATCCGGCCCTGCCGGCTGAAGAAGCGGCCCCCC |
| | | GTGCGG |
| Exemplary | 272 | GCAGCATTCAAACCAAACCCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme2Cas9 | | ACTACGAGCAGCAGCACTAGACCGAAAACTAACACCACTAGAATGATCAGCAGTACTACTACACCTAATCAAACACCGAGGAT |
| HNH | | ACCTATCACAACGAAAAAACGAAGGAGAAACAGCAGACAAAGAACTAGGAGCACTACTAAAAGGAGTAGCAAACAACGCACAC |
| nickase | | GCACTACAAACAGGAGACTTCCGAACACCAGCAGAACTAGCACTAAACAAATTCGAAAAAGAATCAGGACACATCCGAAACCA |
| | | ACGAGGAGACTACTCACACACATTCTCACGAAAAGACCTACAAGCAGAACTAATCCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTAGGACTAGAAGACACAGCATTCTTCAAA |
| | | GGACTACGATACGGAAAAGACAACGCAGAAGCATCAACACTAATGGAAATGAAAGCATACCACGCAATCTCACGAGCACTAGA |
| | | AAAAGAAGGACTAAAAGACAAAAAATCACCACTAAACCTATCATCAGAACTACAAGACGAAATCGGAACAGCATTCTCACTAT |
| | | TCAAAACAGACGAAGACATCACAGGACGACTAAAAGACCGAGTACAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGTACGACTAAACGAAAAAGGA |
| | | TACGTAGAAATCGACGCAGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAATGC |
| | | AACCTAAACGACACACGATACGTAAACCGATTCCTATGCCAATTCGTAGCAGACCACATCCTACTAACAGGAAAAGGAAAACG |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAAAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGAGCACACAAAGACACACTACGATCAGCAAAACGATTCGTAAAACACAACGAAAAAAT |
| | | CTCAGTAAAACGAGTATGACTAACAGAAATCAAACTAGCAGACCTAGAAAACATGGTAAACTACAAAAACGGACGAGAAATCG |
| | | AACTATACGAAGCACTAAAAGCACGACTAGAAGCATACGGAGGAAACGCAAAACAAGCATTCGACCCAAAAGACAACCCATTC |
| | | TACAAAAAAGGAGGACAACTAGTAAAAGCAGTACGAGTAGAAAAAACACAAGAATCAGGAGTACTACTAAACAAAAAAAACGC |
| | | ATACACAATCGCAGACAACGGAGACATGGTACGAGTAGACGTATTCTGCAAAGTAGACAAAAAAGGAAAAAACCAATACTTCA |
| | | TCGTACCAATCTACGCATGACAAGTAGCAGAAAACATCCTACCAGACATCGACTGCAAAGGATACCGAATCGACGACTCATAC |
| | | ACATTCTGCTTCTCACTACACAAATACGACCTAATCGCATTCCAAAAAGACGAAAAATCAAAAGTAGAATTCGCATACTACAT |
| | | CAACTGCGACTCATCAAACGGACGATTCTACCTAGCATGACACGACAAAGGATCAAAAGAACAACAATTCCGAATCTCAACAC |
| | | AAAACCTAGTACTAATCCAAAAATACCAAGTAAACGAACTAGGAAAAGAAATCCGACCATGCCGACTAAAAAAACGACCACCA |
| | | GTACGA |
| Exemplary | 273 | ATGGCTGCTTTTAAGCCTAATCCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme2Cas9 | | GCAGCTTCGTGCTGCTGCTCTTGATCGTAAGCTTACTCCTCTTGAGTGGTCTGCTGTTCTTCTTCATCTTATTAAGCATCGTG |
| HNH | | GTTATCTTTCTCAGCGTAAGAATGAGGGTGAGACTGCTGATAAGGAGCTTGGTGCTCTTCTTAAGGGTGTTGCTAATAATGCT |
| nickase | | CATGCTCTTCAGACTGGTGATTTTCGTACTCCTGCTGAGCTTGCTCTTAATAAGTTTGAGAAGGAGTCTGGTCATATTCGTAA |
| | | TCAGCGTGGTGATTATTCTCATACTTTTTCTCGTAAGGATCTTCAGGCTGAGCTTATTCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTT |
| | | AAGGGTCTTCGTTATGGTAAGGATAATGCTGAGGCTTCTACTCTTATGGAGATGAAGGCTTATCATGCTATTTCTCGTGCTCT |
| | | TGAGAAGGAGGGTCTTAAGGATAAGAAGTCTCCTCTTAATCTTTCTTCTGAGCTTCAGGATGAGATTGGTACTGCTTTTTCTC |
| | | TTTTTAAGACTGATGAGGATATTACTGGTCGTCTTAAGGATCGTGTTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGTTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | TGTAATCTTAATGATACTCGTTATGTTAATCGTTTTCTTTGTCAGTTTGTTGCTGATCATATTCTTCTTACTGGTAAGGGTAA |
| | | GCGTCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTAAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTGCTCATAAGGATACTCTTCGTTCTGCTAAGCGTTTTGTTAAGCATAATGAGAA |
| | | GATTTCTGTTAAGCGTGTTTGGCTTACTGAGATTAAGCTTGCTGATCTTGAGAATATGGTTAATTATAAGAATGGTCGTGAGA |
| | | TTGAGCTTTATGAGGCTCTTAAGGCTCGTCTTGAGGCTTATGGTGGTAATGCTAAGCAGGCTTTTGATCCTAAGGATAATCCT |
| | | TTTTATAAGAAGGGTGGTCAGCTTGTTAAGGCTGTTCGTGTTGAGAAGACTCAGGAGTCTGGTGTTCTTCTTAATAAGAAGAA |
| | | TGCTTATACTATTGCTGATAATGGTGATATGGTTCGTGTTGATGTTTTTTGTAAGGTTGATAAGAAGGGTAAGAATCAGTATT |
| | | TTATTGTTCCTATTTATGCTTGGCAGGTTGCTGAGAATATTCTTCCTGATATTGATTGTAAGGGTTATCGTATTGATGATTCT |
| | | TATACTTTTTGTTTTTCTCTTCATAAGTATGATCTTATTGCTTTTCAGAAGGATGAGAAGTCTAAGGTTGAGTTTGCTTATTA |
| | | TATTAATTGTGATTCTTCTAATGGTCGTTTTTATCTTGCTTGGCATGATAAGGGTTCTAAGGAGCAGCAGTTTCGTATTTCTA |
| | | CTCAGAATCTTGTTCTTATTCAGAAGTATCAGGTTAATGAGCTTGGTAAGGAGATTCGTCCTTGTCGTCTTAAGAAGCGTCCT |
| | | CCTGTTCGTUGA |
| Exemplary | 274 | ATGGCCGCCTTCAAGCCCAACCCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme2Cas9 | | GCAGCTGCGGGCCGCCGCCCTGGACCGGAAGCTGACCCCCCTGGAGTGGTCCGCCGTGCTGCTGCACCTGATCAAGCACCGGG |
| HNH | | GCTACCTGTCCCAGCGGAAGAACGAGGGCGAGACCGCCGACAAGGAGCTGGGCGCCCTGCTGAAGGGCGTGGCCAACAACGCC |
| nickase | | CACGCCCTGCAGACCGGCGACTTCCGGACCCCCGCCGAGCTGGCCCTGAACAAGTTCGAGAAGGAGTCCGGCCACATCCGGAA |
| | | CCAGCGGGGCGACTACTCCCACACCTTCTCCCGGAAGGACCTGCAGGCCGAGCTGATCCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTC |
| | | AAGGGCCTGCGGTACGGCAAGGACAACGCCGAGGCCTCCACCCTGATGGAGATGAAGGCCTACCACGCCATCTCCCGGGCCCT |
| | | GGAGAAGGAGGGCCTGAAGGACAAGAAGTCCCCCCTGAACCTGTCCTCCGAGCTGCAGGACGAGATCGGCACCGCCTTCTCCC |
| | | TGTTCAAGACCGACGAGGACATCACCGGCCGGCTGAAGGACCGGGTGCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGTGCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | TGCAACCTGAACGACACCCGGTACGTGAACCGGTTCCTGTGCCAGTTCGTGGCCGACCACATCCTGCTGACCGGCAAGGGCAA |
| | | GCGGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCAAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCGCCCACAAGGACACCCTGCGGTCCGCCAAGCGGTTCGTGAAGCACAACGAGAA |
| | | GATCTCCGTGAAGCGGGTGTGGCTGACCGAGATCAAGCTGGCCGACCTGGAGAACATGGTGAACTACAAGAACGGCCGGGAGA |
| | | TCGAGCTGTACGAGGCCCTGAAGGCCCGGCTGGAGGCCTACGGCGGCAACGCCAAGCAGGCCTTCGACCCCAAGGACAACCCC |
| | | TTCTACAAGAAGGGCGGCCAGCTGGTGAAGGCCGTGCGGGTGGAGAAGACCCAGGAGTCCGGCGTGCTGCTGAACAAGAAGAA |
| | | CGCCTACACCATCGCCGACAACGGCGACATGGTGCGGGTGGACGTGTTCTGCAAGGTGGACAAGAAGGGCAAGAACCAGTACT |
| | | TCATCGTGCCCATCTACGCCTGGCAGGTGGCCGAGAACATCCTGCCCGACATCGACTGCAAGGGCTACCGGATCGACGACTCC |
| | | TACACCTTCTGCTTCTCCCTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGCAGCAGTTCCGGATCTCCA |
| | | CCCAGAACCTGGTGCTGATCCAGAAGTACCAGGTGAACGAGCTGGGCAAGGAGATCCGGCCCTGCCGGCTGAAGAAGCGGCCC |
| | | CCCGTGCGGUGA |
| Exemplary | 275 | ATGGCAGCATTCAAACCAAACCCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme2Cas9 | | ACAACTACGAGCAGCAGCACTAGACCGAAAACTAACACCACTAGAATGATCAGCAGTACTACTACACCTAATCAAACACCGAG |
| HNH | | GATACCTATCACAACGAAAAAACGAAGGAGAAACAGCAGACAAAGAACTAGGAGCACTACTAAAAGGAGTAGCAAACAACGCA |
| nickase | | CACGCACTACAAACAGGAGACTTCCGAACACCAGCAGAACTAGCACTAAACAAATTCGAAAAAGAATCAGGACACATCCGAAA |
| | | CCAACGAGGAGACTACTCACACACATTCTCACGAAAAGACCTACAAGCAGAACTAATCCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTC |
| | | AAAGGACTACGATACGGAAAAGACAACGCAGAAGCATCAACACTAATGGAAATGAAAGCATACCACGCAATCTCACGAGCACT |
| | | AGAAAAAGAAGGACTAAAAGACAAAAAATCACCACTAAACCTATCATCAGAACTACAAGACGAAATCGGAACAGCATTCTCAC |
| | | TATTCAAAACAGACGAAGACATCACAGGACGACTAAAAGACCGAGTACAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGTACGACTAAACGAAAAA |
| | | GGATACGTAGAAATCGACGCAGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | TGCAACCTAAACGACACACGATACGTAAACCGATTCCTATGCCAATTCGTAGCAGACCACATCCTACTAACAGGAAAAGGAAA |
| | | ACGACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAAAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGAGCACACAAAGACACACTACGATCAGCAAAACGATTCGTAAAACACAACGAAAA |
| | | AATCTCAGTAAAACGAGTATGACTAACAGAAATCAAACTAGCAGACCTAGAAAACATGGTAAACTACAAAAACGGACGAGAAA |
| | | TCGAACTATACGAAGCACTAAAAGCACGACTAGAAGCATACGGAGGAAACGCAAAACAAGCATTCGACCCAAAAGACAACCCA |
| | | TTCTACAAAAAAGGAGGACAACTAGTAAAAGCAGTACGAGTAGAAAAAACACAAGAATCAGGAGTACTACTAAACAAAAAAAA |
| | | CGCATACACAATCGCAGACAACGGAGACATGGTACGAGTAGACGTATTCTGCAAAGTAGACAAAAAAGGAAAAAACCAATACT |
| | | TCATCGTACCAATCTACGCATGACAAGTAGCAGAAAACATCCTACCAGACATCGACTGCAAAGGATACCGAATCGACGACTCA |
| | | TACACATTCTGCTTCTCACTACACAAATACGACCTAATCGCATTCCAAAAAGACGAAAAATCAAAAGTAGAATTCGCATACTA |
| | | CATCAACTGCGACTCATCAAACGGACGATTCTACCTAGCATGACACGACAAAGGATCAAAAGAACAACAATTCCGAATCTCAA |
| | | CACAAAACCTAGTACTAATCCAAAAATACCAAGTAAACGAACTAGGAAAAGAAATCCGACCATGCCGACTAAAAAAACGACCA |
| | | CCAGTACGAUAA |
| Exemplary | 276 | MAAFKPNPINYILGLDIGIASVGWAMVEIDEEENPIRLIDLGVRVFERAEVPKTGDSLAMARRLARSVRRLTRRRAHRLLRAR |
| amino | | RLLKREGVLQAADEDENGLIKSLPNTPWQLRAAALDRKLTPLEWSAVLLHLIKHRGYLSQRKNEGETADKELGALLKGVADNA |
| acid | | HALQTGDFRTPAELALNKFEKECGHIRNQRGDYSHTFSRKDLQAELNLLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLSLEDTAFF |
| of | | KGLRYGKDNAEASTLMEMKAYHTISRALEKEGLKDKKSPLNLSPELQDEIGTAFSLEKTDEDITGRLKDRIQPEILEALLKHI |
| Nme3Cas9 | | SFDKFVQISLKALRRIVPLMEQGKRYDEACAEIYGDHYGKKNTEEKIYLPPIPADEIRNPVVLRALSQARKVINGVVRRYGSP |
| cleavase | | ARIHIETAREVGKSFKDRKEIEKRQEENRKDREKAAAKFREYFPNFVGEPKSKDILKLRLYEQQHGKCLYSGKEINLGRLNEK |
| | | GYVEIDHALPFSRTWDDSFNNKVLVLGSENQNKGNQTPYEYENGKDNSREWQEFKARVETSRFPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITREVRY |
| | | KEMNAFDGKTIDKETGEVLHQKTHFPQPWEFFAQEVMIRVFGKPDGKPEFEEADTPEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVAYADEEDWTVIDESFR |
| | | FKFVLYSNDLIKVQLKKDSFLGYESGLDRATGAISLREHDLEKSKGKDGMHRIGVKTALSFQKYQIDEMGKEIRPCRLKKRPP |
| | | VR |
| Exemplary | 277 | GCTGCTTTTAAGCCTAATCCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme3Cas9 | | GCTTCGTGCTGCTGCTCTTGATCGTAAGCTTACTCCTCTTGAGTGGTCTGCTGTTCTTCTTCATCTTATTAAGCATCGTGGTT |
| cleavase | | ATCTTTCTCAGCGTAAGAATGAGGGTGAGACTGCTGATAAGGAGCTTGGTGCTCTTCTTAAGGGTGTTGCTGATAATGCTCAT |
| | | GCTCTTCAGACTGGTGATTTTCGTACTCCTGCTGAGCTTGCTCTTAATAAGTTTGAGAAGGAGTGTGGTCATATTCGTAATCA |
| | | GCGTGGTGATTATTCTCATACTTTTTCTCGTAAGGATCTTCAGGCTGAGCTTAATCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTTCTCTTGAGGATACTGCTTTTTTTAAG |
| | | GGTCTTCGTTATGGTAAGGATAATGCTGAGGCTTCTACTCTTATGGAGATGAAGGCTTATCATACTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTGCTTATGCTGATGAGGAGGATTGGACTGTTATTGATGAGTCTTTTCGTTTT |
| | | AAGTTTGTTCTTTATTCTAATGATCTTATTAAGGTTCAGCTTAAGAAGGATTCTTTTCTTGGTTATTTTTCTGGTCTTGATCG |
| | | TGCTACTGGTGCTATTTCTCTTCGTGAGCATGATCTTGAGAAGTCTAAGGGTAAGGATGGTATGCATCGTATTGGTGTTAAGA |
| | | CTGCTCTTTCTTTTCAGAAGTATCAGATTGATGAGATGGGTAAGGAGATTCGTCCTTGTCGTCTTAAGAAGCGTCCTCCTGTT |
| | | CGT |
| Exemplary | 278 | GCCGCCTTCAAGCCCAACCCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme3Cas9 | | GCTGCGGGCCGCCGCCCTGGACCGGAAGCTGACCCCCCTGGAGTGGTCCGCCGTGCTGCTGCACCTGATCAAGCACCGGGGCT |
| cleavase | | ACCTGTCCCAGCGGAAGAACGAGGGCGAGACCGCCGACAAGGAGCTGGGCGCCCTGCTGAAGGGCGTGGCCGACAACGCCCAC |
| | | GCCCTGCAGACCGGCGACTTCCGGACCCCCGCCGAGCTGGCCCTGAACAAGTTCGAGAAGGAGTGCGGCCACATCCGGAACCA |
| | | GCGGGGCGACTACTCCCACACCTTCTCCCGGAAGGACCTGCAGGCCGAGCTGAACCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGTCCCTGGAGGACACCGCCTTCTTCAAG |
| | | GGCCTGCGGTACGGCAAGGACAACGCCGAGGCCTCCACCCTGATGGAGATGAAGGCCTACCACACCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGGCCTACGCCGACGAGGAGGACTGGACCGTGATCGACGAGTCCTTCCGGTTC |
| | | AAGTTCGTGCTGTACTCCAACGACCTGATCAAGGTGCAGCTGAAGAAGGACTCCTTCCTGGGCTACTTCTCCGGCCTGGACCG |
| | | GGCCACCGGCGCCATCTCCCTGCGGGAGCACGACCTGGAGAAGTCCAAGGGCAAGGACGGCATGCACCGGATCGGCGTGAAGA |
| | | CCGCCCTGTCCTTCCAGAAGTACCAGATCGACGAGATGGGCAAGGAGATCCGGCCCTGCCGGCTGAAGAAGCGGCCCCCCGTG |
| | | CGG |
| Exemplary | 279 | GCAGCATTCAAACCAAACCCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme3Cas9 | | ACTACGAGCAGCAGCACTAGACCGAAAACTAACACCACTAGAATGATCAGCAGTACTACTACACCTAATCAAACACCGAGGAT |
| cleavase | | ACCTATCACAACGAAAAAACGAAGGAGAAACAGCAGACAAAGAACTAGGAGCACTACTAAAAGGAGTAGCAGACAACGCACAC |
| | | GCACTACAAACAGGAGACTTCCGAACACCAGCAGAACTAGCACTAAACAAATTCGAAAAAGAATGCGGACACATCCGAAACCA |
| | | ACGAGGAGACTACTCACACACATTCTCACGAAAAGACCTACAAGCAGAACTAAACCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTATCACTAGAAGACACAGCATTCTTCAAA |
| | | GGACTACGATACGGAAAAGACAACGCAGAAGCATCAACACTAATGGAAATGAAAGCATACCACACAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACCACGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTAGCATACGCAGACGAAGAAGACTGAACAGTAATCGACGAATCATTCCGATTC |
| | | AAATTCGTACTATACTCAAACGACCTAATCAAAGTACAACTAAAAAAAGACTCATTCCTAGGATACTTCTCAGGACTAGACCG |
| | | AGCAACAGGAGCAATCTCACTACGAGAACACGACCTAGAAAAATCAAAAGGAAAAGACGGAATGCACCGAATCGGAGTAAAAA |
| | | CAGCACTATCATTCCAAAAATACCAAATCGACGAAATGGGAAAAGAAATCCGACCATGCCGACTAAAAAAACGACCACCAGTA |
| | | CGA |
| Exemplary | 280 | ATGGCTGCTTTTAAGCCTAATCCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme3Cas9 | | GCAGCTTCGTGCTGCTGCTCTTGATCGTAAGCTTACTCCTCTTGAGTGGTCTGCTGTTCTTCTTCATCTTATTAAGCATCGTG |
| cleavase | | GTTATCTTTCTCAGCGTAAGAATGAGGGTGAGACTGCTGATAAGGAGCTTGGTGCTCTTCTTAAGGGTGTTGCTGATAATGCT |
| | | CATGCTCTTCAGACTGGTGATTTTCGTACTCCTGCTGAGCTTGCTCTTAATAAGTTTGAGAAGGAGTGTGGTCATATTCGTAA |
| | | TCAGCGTGGTGATTATTCTCATACTTTTTCTCGTAAGGATCTTCAGGCTGAGCTTAATCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTTCTCTTGAGGATACTGCTTTTTTT |
| | | AAGGGTCTTCGTTATGGTAAGGATAATGCTGAGGCTTCTACTCTTATGGAGATGAAGGCTTATCATACTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTGCTTATGCTGATGAGGAGGATTGGACTGTTATTGATGAGTCTTTTCGT |
| | | TTTAAGTTTGTTCTTTATTCTAATGATCTTATTAAGGTTCAGCTTAAGAAGGATTCTTTTCTTGGTTATTTTTCTGGTCTTGA |
| | | TCGTGCTACTGGTGCTATTTCTCTTCGTGAGCATGATCTTGAGAAGTCTAAGGGTAAGGATGGTATGCATCGTATTGGTGTTA |
| | | AGACTGCTCTTTCTTTTCAGAAGTATCAGATTGATGAGATGGGTAAGGAGATTCGTCCTTGTCGTCTTAAGAAGCGTCCTCCT |
| | | GTTCGTUGA |
| Exemplary | 281 | ATGGCCGCCTTCAAGCCCAACCCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme3Cas9 | | GCAGCTGCGGGCCGCCGCCCTGGACCGGAAGCTGACCCCCCTGGAGTGGTCCGCCGTGCTGCTGCACCTGATCAAGCACCGGG |
| cleavase | | GCTACCTGTCCCAGCGGAAGAACGAGGGCGAGACCGCCGACAAGGAGCTGGGCGCCCTGCTGAAGGGCGTGGCCGACAACGCC |
| | | CACGCCCTGCAGACCGGCGACTTCCGGACCCCCGCCGAGCTGGCCCTGAACAAGTTCGAGAAGGAGTGCGGCCACATCCGGAA |
| | | CCAGCGGGGCGACTACTCCCACACCTTCTCCCGGAAGGACCTGCAGGCCGAGCTGAACCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGTCCCTGGAGGACACCGCCTTCTTC |
| | | AAGGGCCTGCGGTACGGCAAGGACAACGCCGAGGCCTCCACCCTGATGGAGATGAAGGCCTACCACACCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGGCCTACGCCGACGAGGAGGACTGGACCGTGATCGACGAGTCCTTCCGG |
| | | TTCAAGTTCGTGCTGTACTCCAACGACCTGATCAAGGTGCAGCTGAAGAAGGACTCCTTCCTGGGCTACTTCTCCGGCCTGGA |
| | | CCGGGCCACCGGCGCCATCTCCCTGCGGGAGCACGACCTGGAGAAGTCCAAGGGCAAGGACGGCATGCACCGGATCGGCGTGA |
| | | AGACCGCCCTGTCCTTCCAGAAGTACCAGATCGACGAGATGGGCAAGGAGATCCGGCCCTGCCGGCTGAAGAAGCGGCCCCCC |
| | | GTGCGGUGA |
| Exemplary | 282 | ATGGCAGCATTCAAACCAAACCCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme3Cas9 | | ACAACTACGAGCAGCAGCACTAGACCGAAAACTAACACCACTAGAATGATCAGCAGTACTACTACACCTAATCAAACACCGAG |
| cleavase | | GATACCTATCACAACGAAAAAACGAAGGAGAAACAGCAGACAAAGAACTAGGAGCACTACTAAAAGGAGTAGCAGACAACGCA |
| | | CACGCACTACAAACAGGAGACTTCCGAACACCAGCAGAACTAGCACTAAACAAATTCGAAAAAGAATGCGGACACATCCGAAA |
| | | CCAACGAGGAGACTACTCACACACATTCTCACGAAAAGACCTACAAGCAGAACTAAACCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTATCACTAGAAGACACAGCATTCTTC |
| | | AAAGGACTACGATACGGAAAAGACAACGCAGAAGCATCAACACTAATGGAAATGAAAGCATACCACACAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACCACGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTAGCATACGCAGACGAAGAAGACTGAACAGTAATCGACGAATCATTCCGA |
| | | TTCAAATTCGTACTATACTCAAACGACCTAATCAAAGTACAACTAAAAAAAGACTCATTCCTAGGATACTTCTCAGGACTAGA |
| | | CCGAGCAACAGGAGCAATCTCACTACGAGAACACGACCTAGAAAAATCAAAAGGAAAAGACGGAATGCACCGAATCGGAGTAA |
| | | AAACAGCACTATCATTCCAAAAATACCAAATCGACGAAATGGGAAAAGAAATCCGACCATGCCGACTAAAAAAACGACCACCA |
| | | GTACGAUAA |
| Exemplary | 283 | MAAFKPNPINYILGLAIGIASVGWAMVEIDEEENPIRLIDLGVRVFERAEVPKTGDSLAMARRLARSVRRLTRRRAHRLLRAR |
| amino | | RLLKREGVLQAADFDENGLIKSLPNTPWQLRAAALDRKLTPLEWSAVLLHLIKHRGYLSQRKNEGETADKELGALLKGVADNA |
| acid | | HALQTGDFRTPAELALNKFEKECGHIRNQRGDYSHTFSRKDLQAELNLLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLSLEDTAFF |
| of | | KGLRYGKDNAEASTLMEMKAYHTISRALEKEGLKDKKSPLNLSPELQDEIGTAFSLEKTDEDITGRLKDRIQPEILEALLKHI |
| Nme3Cas9 | | SFDKFVQISLKALRRIVPLMEQGKRYDEACAEIYGDHYGKKNTEEKIYLPPIPADEIRNPVVLRALSQARKVINGVVRRYGSP |
| dCas9 | | ARIHIETAREVGKSFKDRKEIEKRQEENRKDREKAAAKFREYFPNFVGEPKSKDILKLRLYEQQHGKCLYSGKEINLGRLNEK |
| | | GYVEIDAALPESRTWDDSFNNKVLVLGSENQNKGNQTPYEYENGKDNSREWQEFKARVETSREPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHEPQPWEFFAQEVMIRVFGKPDGKPEFEEADTPEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVAYADEEDWTVIDESFR |
| | | FKFVLYSNDLIKVQLKKDSFLGYFSGLDRATGAISLREHDLEKSKGKDGMHRIGVKTALSFQKYQIDEMGKEIRPCRLKKRPP |
| | | VR |
| Exemplary | 284 | GCTGCTTTTAAGCCTAATCCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme3Cas9 | | GCTTCGTGCTGCTGCTCTTGATCGTAAGCTTACTCCTCTTGAGTGGTCTGCTGTTCTTCTTCATCTTATTAAGCATCGTGGTT |
| dCas9 | | ATCTTTCTCAGCGTAAGAATGAGGGTGAGACTGCTGATAAGGAGCTTGGTGCTCTTCTTAAGGGTGTTGCTGATAATGCTCAT |
| | | GCTCTTCAGACTGGTGATTTTCGTACTCCTGCTGAGCTTGCTCTTAATAAGTTTGAGAAGGAGTGTGGTCATATTCGTAATCA |
| | | GCGTGGTGATTATTCTCATACTTTTTCTCGTAAGGATCTTCAGGCTGAGCTTAATCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTTCTCTTGAGGATACTGCTTTTTTTAAG |
| | | GGTCTTCGTTATGGTAAGGATAATGCTGAGGCTTCTACTCTTATGGAGATGAAGGCTTATCATACTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTGCTTATGCTGATGAGGAGGATTGGACTGTTATTGATGAGTCTTTTCGTTTT |
| | | AAGTTTGTTCTTTATTCTAATGATCTTATTAAGGTTCAGCTTAAGAAGGATTCTTTTCTTGGTTATTTTTCTGGTCTTGATCG |
| | | TGCTACTGGTGCTATTTCTCTTCGTGAGCATGATCTTGAGAAGTCTAAGGGTAAGGATGGTATGCATCGTATTGGTGTTAAGA |
| | | CTGCTCTTTCTTTTCAGAAGTATCAGATTGATGAGATGGGTAAGGAGATTCGTCCTTGTCGTCTTAAGAAGCGTCCTCCTGTT |
| | | CGT |
| Exemplary | 285 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme3Cas9 | | GCTGCGGGCCGCCGCCCTGGACCGGAAGCTGACCCCCCTGGAGTGGTCCGCCGTGCTGCTGCACCTGATCAAGCACCGGGGCT |
| dCas9 | | ACCTGTCCCAGCGGAAGAACGAGGGCGAGACCGCCGACAAGGAGCTGGGCGCCCTGCTGAAGGGCGTGGCCGACAACGCCCAC |
| | | GCCCTGCAGACCGGCGACTTCCGGACCCCCGCCGAGCTGGCCCTGAACAAGTTCGAGAAGGAGTGCGGCCACATCCGGAACCA |
| | | GCGGGGCGACTACTCCCACACCTTCTCCCGGAAGGACCTGCAGGCCGAGCTGAACCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGTCCCTGGAGGACACCGCCTTCTTCAAG |
| | | GGCCTGCGGTACGGCAAGGACAACGCCGAGGCCTCCACCCTGATGGAGATGAAGGCCTACCACACCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGGCCTACGCCGACGAGGAGGACTGGACCGTGATCGACGAGTCCTTCCGGTTC |
| | | AAGTTCGTGCTGTACTCCAACGACCTGATCAAGGTGCAGCTGAAGAAGGACTCCTTCCTGGGCTACTTCTCCGGCCTGGACCG |
| | | GGCCACCGGCGCCATCTCCCTGCGGGAGCACGACCTGGAGAAGTCCAAGGGCAAGGACGGCATGCACCGGATCGGCGTGAAGA |
| | | CCGCCCTGTCCTTCCAGAAGTACCAGATCGACGAGATGGGCAAGGAGATCCGGCCCTGCCGGCTGAAGAAGCGGCCCCCCGTG |
| | | CGG |
| Exemplary | 286 | GCAGCATTCAAACCAAACCCAATCAACTACATCCTAGGACTAGCAATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme3Cas9 | | ACTACGAGCAGCAGCACTAGACCGAAAACTAACACCACTAGAATGATCAGCAGTACTACTACACCTAATCAAACACCGAGGAT |
| dCas9 | | ACCTATCACAACGAAAAAACGAAGGAGAAACAGCAGACAAAGAACTAGGAGCACTACTAAAAGGAGTAGCAGACAACGCACAC |
| | | GCACTACAAACAGGAGACTTCCGAACACCAGCAGAACTAGCACTAAACAAATTCGAAAAAGAATGCGGACACATCCGAAACCA |
| | | ACGAGGAGACTACTCACACACATTCTCACGAAAAGACCTACAAGCAGAACTAAACCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTATCACTAGAAGACACAGCATTCTTCAAA |
| | | GGACTACGATACGGAAAAGACAACGCAGAAGCATCAACACTAATGGAAATGAAAGCATACCACACAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACGCAGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTAGCATACGCAGACGAAGAAGACTGAACAGTAATCGACGAATCATTCCGATTC |
| | | AAATTCGTACTATACTCAAACGACCTAATCAAAGTACAACTAAAAAAAGACTCATTCCTAGGATACTTCTCAGGACTAGACCG |
| | | AGCAACAGGAGCAATCTCACTACGAGAACACGACCTAGAAAAATCAAAAGGAAAAGACGGAATGCACCGAATCGGAGTAAAAA |
| | | CAGCACTATCATTCCAAAAATACCAAATCGACGAAATGGGAAAAGAAATCCGACCATGCCGACTAAAAAAACGACCACCAGTA |
| | | CGA |
| Exemplary | 287 | ATGGCTGCTTTTAAGCCTAATCCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme3Cas9 | | GCAGCTTCGTGCTGCTGCTCTTGATCGTAAGCTTACTCCTCTTGAGTGGTCTGCTGTTCTTCTTCATCTTATTAAGCATCGTG |
| dCas9 | | GTTATCTTTCTCAGCGTAAGAATGAGGGTGAGACTGCTGATAAGGAGCTTGGTGCTCTTCTTAAGGGTGTTGCTGATAATGCT |
| | | CATGCTCTTCAGACTGGTGATTTTCGTACTCCTGCTGAGCTTGCTCTTAATAAGTTTGAGAAGGAGTGTGGTCATATTCGTAA |
| | | TCAGCGTGGTGATTATTCTCATACTTTTTCTCGTAAGGATCTTCAGGCTGAGCTTAATCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTTCTCTTGAGGATACTGCTTTTTTT |
| | | AAGGGTCTTCGTTATGGTAAGGATAATGCTGAGGCTTCTACTCTTATGGAGATGAAGGCTTATCATACTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTGCTTATGCTGATGAGGAGGATTGGACTGTTATTGATGAGTCTTTTCGT |
| | | TTTAAGTTTGTTCTTTATTCTAATGATCTTATTAAGGTTCAGCTTAAGAAGGATTCTTTTCTTGGTTATTTTTCTGGTCTTGA |
| | | TCGTGCTACTGGTGCTATTTCTCTTCGTGAGCATGATCTTGAGAAGTCTAAGGGTAAGGATGGTATGCATCGTATTGGTGTTA |
| | | AGACTGCTCTTTCTTTTCAGAAGTATCAGATTGATGAGATGGGTAAGGAGATTCGTCCTTGTCGTCTTAAGAAGCGTCCTCCT |
| | | GTTCGTUGA |
| Exemplary | 288 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme3Cas9 | | GCAGCTGCGGGCCGCCGCCCTGGACCGGAAGCTGACCCCCCTGGAGTGGTCCGCCGTGCTGCTGCACCTGATCAAGCACCGGG |
| dCas9 | | GCTACCTGTCCCAGCGGAAGAACGAGGGCGAGACCGCCGACAAGGAGCTGGGCGCCCTGCTGAAGGGCGTGGCCGACAACGCC |
| | | CACGCCCTGCAGACCGGCGACTTCCGGACCCCCGCCGAGCTGGCCCTGAACAAGTTCGAGAAGGAGTGCGGCCACATCCGGAA |
| | | CCAGCGGGGCGACTACTCCCACACCTTCTCCCGGAAGGACCTGCAGGCCGAGCTGAACCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGTCCCTGGAGGACACCGCCTTCTTC |
| | | AAGGGCCTGCGGTACGGCAAGGACAACGCCGAGGCCTCCACCCTGATGGAGATGAAGGCCTACCACACCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGGCCTACGCCGACGAGGAGGACTGGACCGTGATCGACGAGTCCTTCCGG |
| | | TTCAAGTTCGTGCTGTACTCCAACGACCTGATCAAGGTGCAGCTGAAGAAGGACTCCTTCCTGGGCTACTTCTCCGGCCTGGA |
| | | CCGGGCCACCGGCGCCATCTCCCTGCGGGAGCACGACCTGGAGAAGTCCAAGGGCAAGGACGGCATGCACCGGATCGGCGTGA |
| | | AGACCGCCCTGTCCTTCCAGAAGTACCAGATCGACGAGATGGGCAAGGAGATCCGGCCCTGCCGGCTGAAGAAGCGGCCCCCC |
| | | GTGCGGUGA |
| Exemplary | 289 | ATGGCAGCATTCAAACCAAACCCAATCAACTACATCCTAGGACTAGCAATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme3Cas9 | | ACAACTACGAGCAGCAGCACTAGACCGAAAACTAACACCACTAGAATGATCAGCAGTACTACTACACCTAATCAAACACCGAG |
| dCas9 | | GATACCTATCACAACGAAAAAACGAAGGAGAAACAGCAGACAAAGAACTAGGAGCACTACTAAAAGGAGTAGCAGACAACGCA |
| | | CACGCACTACAAACAGGAGACTTCCGAACACCAGCAGAACTAGCACTAAACAAATTCGAAAAAGAATGCGGACACATCCGAAA |
| | | CCAACGAGGAGACTACTCACACACATTCTCACGAAAAGACCTACAAGCAGAACTAAACCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTATCACTAGAAGACACAGCATTCTTC |
| | | AAAGGACTACGATACGGAAAAGACAACGCAGAAGCATCAACACTAATGGAAATGAAAGCATACCACACAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACGCAGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTAGCATACGCAGACGAAGAAGACTGAACAGTAATCGACGAATCATTCCGA |
| | | TTCAAATTCGTACTATACTCAAACGACCTAATCAAAGTACAACTAAAAAAAGACTCATTCCTAGGATACTTCTCAGGACTAGA |
| | | CCGAGCAACAGGAGCAATCTCACTACGAGAACACGACCTAGAAAAATCAAAAGGAAAAGACGGAATGCACCGAATCGGAGTAA |
| | | AAACAGCACTATCATTCCAAAAATACCAAATCGACGAAATGGGAAAAGAAATCCGACCATGCCGACTAAAAAAACGACCACCA |
| | | GTACGAUAA |
| Exemplary | 290 | MAAFKPNPINYILGLAIGIASVGWAMVEIDEEENPIRLIDLGVRVFERAEVPKTGDSLAMARRLARSVRRLTRRRAHRLLRAR |
| amino | | RLLKREGVLQAADFDENGLIKSLPNTPWQLRAAALDRKLTPLEWSAVLLHLIKHRGYLSQRKNEGETADKELGALLKGVADNA |
| acid | | HALQTGDFRTPAELALNKFEKECGHIRNQRGDYSHTFSRKDLQAELNLLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLSLEDTAFF |
| of | | KGLRYGKDNAEASTLMEMKAYHTISRALEKEGLKDKKSPLNLSPELQDEIGTAFSLEKTDEDITGRLKDRIQPEILEALLKHI |
| Nme3Cas9 | | SFDKFVQISLKALRRIVPLMEQGKRYDEACAEIYGDHYGKKNTEEKIYLPPIPADEIRNPVVLRALSQARKVINGVVRRYGSP |
| RuvC | | ARIHIETAREVGKSFKDRKEIEKRQEENRKDREKAAAKFREYFPNFVGEPKSKDILKLRLYEQQHGKCLYSGKEINLGRLNEK |
| nickase | | GYVEIDHALPFSRTWDDSFNNKVLVLGSENQNKGNQTPYEYENGKDNSREWQEFKARVETSRFPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHEPQPWEFFAQEVMIRVFGKPDGKPEFEEADTPEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVAYADEEDWTVIDESFR |
| | | FKFVLYSNDLIKVQLKKDSFLGYESGLDRATGAISLREHDLEKSKGKDGMHRIGVKTALSFQKYQIDEMGKEIRPCRLKKRPP |
| | | VR |
| Exemplary | 291 | GCTGCTTTTAAGCCTAATCCTATTAATTATATTCTTGGTCTTGCTATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme3Cas9 | | GCTTCGTGCTGCTGCTCTTGATCGTAAGCTTACTCCTCTTGAGTGGTCTGCTGTTCTTCTTCATCTTATTAAGCATCGTGGTT |
| RuvC | | ATCTTTCTCAGCGTAAGAATGAGGGTGAGACTGCTGATAAGGAGCTTGGTGCTCTTCTTAAGGGTGTTGCTGATAATGCTCAT |
| nickase | | GCTCTTCAGACTGGTGATTTTCGTACTCCTGCTGAGCTTGCTCTTAATAAGTTTGAGAAGGAGTGTGGTCATATTCGTAATCA |
| | | GCGTGGTGATTATTCTCATACTTTTTCTCGTAAGGATCTTCAGGCTGAGCTTAATCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTTCTCTTGAGGATACTGCTTTTTTTAAG |
| | | GGTCTTCGTTATGGTAAGGATAATGCTGAGGCTTCTACTCTTATGGAGATGAAGGCTTATCATACTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATCATGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTGCTTATGCTGATGAGGAGGATTGGACTGTTATTGATGAGTCTTTTCGTTTT |
| | | AAGTTTGTTCTTTATTCTAATGATCTTATTAAGGTTCAGCTTAAGAAGGATTCTTTTCTTGGTTATTTTTCTGGTCTTGATCG |
| | | TGCTACTGGTGCTATTTCTCTTCGTGAGCATGATCTTGAGAAGTCTAAGGGTAAGGATGGTATGCATCGTATTGGTGTTAAGA |
| | | CTGCTCTTTCTTTTCAGAAGTATCAGATTGATGAGATGGGTAAGGAGATTCGTCCTTGTCGTCTTAAGAAGCGTCCTCCTGTT |
| | | CGT |
| Exemplary | 292 | GCCGCCTTCAAGCCCAACCCCATCAACTACATCCTGGGCCTGGCCATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme3Cas9 | | GCTGCGGGCCGCCGCCCTGGACCGGAAGCTGACCCCCCTGGAGTGGTCCGCCGTGCTGCTGCACCTGATCAAGCACCGGGGCT |
| RuvC | | ACCTGTCCCAGCGGAAGAACGAGGGCGAGACCGCCGACAAGGAGCTGGGCGCCCTGCTGAAGGGCGTGGCCGACAACGCCCAC |
| nickase | | GCCCTGCAGACCGGCGACTTCCGGACCCCCGCCGAGCTGGCCCTGAACAAGTTCGAGAAGGAGTGCGGCCACATCCGGAACCA |
| | | GCGGGGCGACTACTCCCACACCTTCTCCCGGAAGGACCTGCAGGCCGAGCTGAACCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGTCCCTGGAGGACACCGCCTTCTTCAAG |
| | | GGCCTGCGGTACGGCAAGGACAACGCCGAGGCCTCCACCCTGATGGAGATGAAGGCCTACCACACCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACCACGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGGCCTACGCCGACGAGGAGGACTGGACCGTGATCGACGAGTCCTTCCGGTTC |
| | | AAGTTCGTGCTGTACTCCAACGACCTGATCAAGGTGCAGCTGAAGAAGGACTCCTTCCTGGGCTACTTCTCCGGCCTGGACCG |
| | | GGCCACCGGCGCCATCTCCCTGCGGGAGCACGACCTGGAGAAGTCCAAGGGCAAGGACGGCATGCACCGGATCGGCGTGAAGA |
| | | CCGCCCTGTCCTTCCAGAAGTACCAGATCGACGAGATGGGCAAGGAGATCCGGCCCTGCCGGCTGAAGAAGCGGCCCCCCGTG |
| | | CGG |
| Exemplary | 293 | GCAGCATTCAAACCAAACCCAATCAACTACATCCTAGGACTAGCAATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme3Cas9 | | ACTACGAGCAGCAGCACTAGACCGAAAACTAACACCACTAGAATGATCAGCAGTACTACTACACCTAATCAAACACCGAGGAT |
| Ruvc | | ACCTATCACAACGAAAAAACGAAGGAGAAACAGCAGACAAAGAACTAGGAGCACTACTAAAAGGAGTAGCAGACAACGCACAC |
| nickase | | GCACTACAAACAGGAGACTTCCGAACACCAGCAGAACTAGCACTAAACAAATTCGAAAAAGAATGCGGACACATCCGAAACCA |
| | | ACGAGGAGACTACTCACACACATTCTCACGAAAAGACCTACAAGCAGAACTAAACCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTATCACTAGAAGACACAGCATTCTTCAAA |
| | | GGACTACGATACGGAAAAGACAACGCAGAAGCATCAACACTAATGGAAATGAAAGCATACCACACAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACCACGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTAGCATACGCAGACGAAGAAGACTGAACAGTAATCGACGAATCATTCCGATTC |
| | | AAATTCGTACTATACTCAAACGACCTAATCAAAGTACAACTAAAAAAAGACTCATTCCTAGGATACTTCTCAGGACTAGACCG |
| | | AGCAACAGGAGCAATCTCACTACGAGAACACGACCTAGAAAAATCAAAAGGAAAAGACGGAATGCACCGAATCGGAGTAAAAA |
| | | CAGCACTATCATTCCAAAAATACCAAATCGACGAAATGGGAAAAGAAATCCGACCATGCCGACTAAAAAAACGACCACCAGTA |
| | | CGA |
| Exemplary | 294 | ATGGCTGCTTTTAAGCCTAATCCTATTAATTATATTCTTGGTCTTGCTATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme3Cas9 | | GCAGCTTCGTGCTGCTGCTCTTGATCGTAAGCTTACTCCTCTTGAGTGGTCTGCTGTTCTTCTTCATCTTATTAAGCATCGTG |
| RuvC | | GTTATCTTTCTCAGCGTAAGAATGAGGGTGAGACTGCTGATAAGGAGCTTGGTGCTCTTCTTAAGGGTGTTGCTGATAATGCT |
| nickase | | CATGCTCTTCAGACTGGTGATTTTCGTACTCCTGCTGAGCTTGCTCTTAATAAGTTTGAGAAGGAGTGTGGTCATATTCGTAA |
| | | TCAGCGTGGTGATTATTCTCATACTTTTTCTCGTAAGGATCTTCAGGCTGAGCTTAATCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTTCTCTTGAGGATACTGCTTTTTTT |
| | | AAGGGTCTTCGTTATGGTAAGGATAATGCTGAGGCTTCTACTCTTATGGAGATGAAGGCTTATCATACTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATCATGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTGCTTATGCTGATGAGGAGGATTGGACTGTTATTGATGAGTCTTTTCGT |
| | | TTTAAGTTTGTTCTTTATTCTAATGATCTTATTAAGGTTCAGCTTAAGAAGGATTCTTTTCTTGGTTATTTTTCTGGTCTTGA |
| | | TCGTGCTACTGGTGCTATTTCTCTTCGTGAGCATGATCTTGAGAAGTCTAAGGGTAAGGATGGTATGCATCGTATTGGTGTTA |
| | | AGACTGCTCTTTCTTTTCAGAAGTATCAGATTGATGAGATGGGTAAGGAGATTCGTCCTTGTCGTCTTAAGAAGCGTCCTCCT |
| | | GTTCGTUAA |
| Exemplary | 295 | ATGGCCGCCTTCAAGCCCAACCCCATCAACTACATCCTGGGCCTGGCCATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme3Cas9 | | GCAGCTGCGGGCCGCCGCCCTGGACCGGAAGCTGACCCCCCTGGAGTGGTCCGCCGTGCTGCTGCACCTGATCAAGCACCGGG |
| RuvC | | GCTACCTGTCCCAGCGGAAGAACGAGGGCGAGACCGCCGACAAGGAGCTGGGCGCCCTGCTGAAGGGCGTGGCCGACAACGCC |
| nickase | | CACGCCCTGCAGACCGGCGACTTCCGGACCCCCGCCGAGCTGGCCCTGAACAAGTTCGAGAAGGAGTGCGGCCACATCCGGAA |
| | | CCAGCGGGGCGACTACTCCCACACCTTCTCCCGGAAGGACCTGCAGGCCGAGCTGAACCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGTCCCTGGAGGACACCGCCTTCTTC |
| | | AAGGGCCTGCGGTACGGCAAGGACAACGCCGAGGCCTCCACCCTGATGGAGATGAAGGCCTACCACACCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACCACGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGGCCTACGCCGACGAGGAGGACTGGACCGTGATCGACGAGTCCTTCCGG |
| | | TTCAAGTTCGTGCTGTACTCCAACGACCTGATCAAGGTGCAGCTGAAGAAGGACTCCTTCCTGGGCTACTTCTCCGGCCTGGA |
| | | CCGGGCCACCGGCGCCATCTCCCTGCGGGAGCACGACCTGGAGAAGTCCAAGGGCAAGGACGGCATGCACCGGATCGGCGTGA |
| | | AGACCGCCCTGTCCTTCCAGAAGTACCAGATCGACGAGATGGGCAAGGAGATCCGGCCCTGCCGGCTGAAGAAGCGGCCCCCC |
| | | GTGCGGUGA |
| Exemplary | 296 | ATGGCAGCATTCAAACCAAACCCAATCAACTACATCCTAGGACTAGCAATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme3Cas9 | | ACAACTACGAGCAGCAGCACTAGACCGAAAACTAACACCACTAGAATGATCAGCAGTACTACTACACCTAATCAAACACCGAG |
| Ruvc | | GATACCTATCACAACGAAAAAACGAAGGAGAAACAGCAGACAAAGAACTAGGAGCACTACTAAAAGGAGTAGCAGACAACGCA |
| nickase | | CACGCACTACAAACAGGAGACTTCCGAACACCAGCAGAACTAGCACTAAACAAATTCGAAAAAGAATGCGGACACATCCGAAA |
| | | CCAACGAGGAGACTACTCACACACATTCTCACGAAAAGACCTACAAGCAGAACTAAACCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTATCACTAGAAGACACAGCATTCTTC |
| | | AAAGGACTACGATACGGAAAAGACAACGCAGAAGCATCAACACTAATGGAAATGAAAGCATACCACACAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACCACGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTAGCATACGCAGACGAAGAAGACTGAACAGTAATCGACGAATCATTCCGA |
| | | TTCAAATTCGTACTATACTCAAACGACCTAATCAAAGTACAACTAAAAAAAGACTCATTCCTAGGATACTTCTCAGGACTAGA |
| | | CCGAGCAACAGGAGCAATCTCACTACGAGAACACGACCTAGAAAAATCAAAAGGAAAAGACGGAATGCACCGAATCGGAGTAA |
| | | AAACAGCACTATCATTCCAAAAATACCAAATCGACGAAATGGGAAAAGAAATCCGACCATGCCGACTAAAAAAACGACCACCA |
| | | GTACGAUAA |
| Exemplary | 297 | MAAFKPNPINYILGLDIGIASVGWAMVEIDEEENPIRLIDLGVRVFERAEVPKTGDSLAMARRLARSVRRLTRRRAHRLLRAR |
| amino | | RLLKREGVLQAADEDENGLIKSLPNTPWQLRAAALDRKLTPLEWSAVLLHLIKHRGYLSQRKNEGETADKELGALLKGVADNA |
| acid | | HALQTGDFRTPAELALNKFEKECGHIRNQRGDYSHTFSRKDLQAELNLLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLSLEDTAFF |
| of | | KGLRYGKDNAEASTLMEMKAYHTISRALEKEGLKDKKSPLNLSPELQDEIGTAFSLFKTDEDITGRLKDRIQPEILEALLKHI |
| Nme3Cas9 | | SFDKFVQISLKALRRIVPLMEQGKRYDEACAEIYGDHYGKKNTEEKIYLPPIPADEIRNPVVLRALSQARKVINGVVRRYGSP |
| HNH | | ARIHIETAREVGKSFKDRKEIEKRQEENRKDREKAAAKFREYFPNFVGEPKSKDILKLRLYEQQHGKCLYSGKEINLGRLNEK |
| nickase | | GYVEIDAALPESRTWDDSFNNKVLVLGSENQNKGNQTPYEYENGKDNSREWQEFKARVETSREPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITREVRY |
| | | KEMNAFDGKTIDKETGEVLHQKTHEPQPWEFFAQEVMIRVFGKPDGKPEFEEADTPEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVAYADEEDWTVIDESFR |
| | | FKFVLYSNDLIKVQLKKDSFLGYFSGLDRATGAISLREHDLEKSKGKDGMHRIGVKTALSFQKYQIDEMGKEIRPCRLKKRPP |
| | | VR |
| Exemplary | 298 | GCTGCTTTTAAGCCTAATCCTATTAATTATATTCTTGGTCTTGATATTGGTATTGCTTCTGTTGGTTGGGCTATGGTTGAGAT |
| coding | | TGATGAGGAGGAGAATCCTATTCGTCTTATTGATCTTGGTGTTCGTGTTTTTGAGCGTGCTGAGGTTCCTAAGACTGGTGATT |
| sequence | | CTCTTGCTATGGCTCGTCGTCTTGCTCGTTCTGTTCGTCGTCTTACTCGTCGTCGTGCTCATCGTCTTCTTCGTGCTCGTCGT |
| encoding | | CTTCTTAAGCGTGAGGGTGTTCTTCAGGCTGCTGATTTTGATGAGAATGGTCTTATTAAGTCTCTTCCTAATACTCCTTGGCA |
| Nme3Cas9 | | GCTTCGTGCTGCTGCTCTTGATCGTAAGCTTACTCCTCTTGAGTGGTCTGCTGTTCTTCTTCATCTTATTAAGCATCGTGGTT |
| HNH | | ATCTTTCTCAGCGTAAGAATGAGGGTGAGACTGCTGATAAGGAGCTTGGTGCTCTTCTTAAGGGTGTTGCTGATAATGCTCAT |
| nickase | | GCTCTTCAGACTGGTGATTTTCGTACTCCTGCTGAGCTTGCTCTTAATAAGTTTGAGAAGGAGTGTGGTCATATTCGTAATCA |
| | | GCGTGGTGATTATTCTCATACTTTTTCTCGTAAGGATCTTCAGGCTGAGCTTAATCTTCTTTTTGAGAAGCAGAAGGAGTTTG |
| | | GTAATCCTCATGTTTCTGGTGGTCTTAAGGAGGGTATTGAGACTCTTCTTATGACTCAGCGTCCTGCTCTTTCTGGTGATGCT |
| | | GTTCAGAAGATGCTTGGTCATTGTACTTTTGAGCCTGCTGAGCCTAAGGCTGCTAAGAATACTTATACTGCTGAGCGTTTTAT |
| | | TTGGCTTACTAAGCTTAATAATCTTCGTATTCTTGAGCAGGGTTCTGAGCGTCCTCTTACTGATACTGAGCGTGCTACTCTTA |
| | | TGGATGAGCCTTATCGTAAGTCTAAGCTTACTTATGCTCAGGCTCGTAAGCTTCTTTCTCTTGAGGATACTGCTTTTTTTAAG |
| | | GGTCTTCGTTATGGTAAGGATAATGCTGAGGCTTCTACTCTTATGGAGATGAAGGCTTATCATACTATTTCTCGTGCTCTTGA |
| | | GAAGGAGGGTCTTAAGGATAAGAAGTCTCCTCTTAATCTTTCTCCTGAGCTTCAGGATGAGATTGGTACTGCTTTTTCTCTTT |
| | | TTAAGACTGATGAGGATATTACTGGTCGTCTTAAGGATCGTATTCAGCCTGAGATTCTTGAGGCTCTTCTTAAGCATATTTCT |
| | | TTTGATAAGTTTGTTCAGATTTCTCTTAAGGCTCTTCGTCGTATTGTTCCTCTTATGGAGCAGGGTAAGCGTTATGATGAGGC |
| | | TTGTGCTGAGATTTATGGTGATCATTATGGTAAGAAGAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTATTAATGGTGTTGTTCGTCGTTATGGTTCTCCTGCT |
| | | CGTATTCATATTGAGACTGCTCGTGAGGTTGGTAAGTCTTTTAAGGATCGTAAGGAGATTGAGAAGCGTCAGGAGGAGAATCG |
| | | TAAGGATCGTGAGAAGGCTGCTGCTAAGTTTCGTGAGTATTTTCCTAATTTTGTTGGTGAGCCTAAGTCTAAGGATATTCTTA |
| | | AGCTTCGTCTTTATGAGCAGCAGCATGGTAAGTGTCTTTATTCTGGTAAGGAGATTAATCTTGGTCGTCTTAATGAGAAGGGT |
| | | TATGTTGAGATTGATGCTGCTCTTCCTTTTTCTCGTACTTGGGATGATTCTTTTAATAATAAGGTTCTTGTTCTTGGTTCTGA |
| | | GAATCAGAATAAGGGTAATCAGACTCCTTATGAGTATTTTAATGGTAAGGATAATTCTCGTGAGTGGCAGGAGTTTAAGGCTC |
| | | GTGTTGAGACTTCTCGTTTTCCTCGTTCTAAGAAGCAGCGTATTCTTCTTCAGAAGTTTGATGAGGATGGTTTTAAGGAGCGT |
| | | AATCTTAATGATACTCGTTATGTTAATCGTTTTCTTTGTCAGTTTGTTGCTGATCGTATGCGTCTTACTGGTAAGGGTAAGAA |
| | | GCGTGTTTTTGCTTCTAATGGTCAGATTACTAATCTTCTTCGTGGTTTTTGGGGTCTTCGTAAGGTTCGTGCTGAGAATGATC |
| | | GTCATCATGCTCTTGATGCTGTTGTTGTTGCTTGTTCTACTGTTGCTATGCAGCAGAAGATTACTCGTTTTGTTCGTTATAAG |
| | | GAGATGAATGCTTTTGATGGTAAGACTATTGATAAGGAGACTGGTGAGGTTCTTCATCAGAAGACTCATTTTCCTCAGCCTTG |
| | | GGAGTTTTTTGCTCAGGAGGTTATGATTCGTGTTTTTGGTAAGCCTGATGGTAAGCCTGAGTTTGAGGAGGCTGATACTCCTG |
| | | AGAAGCTTCGTACTCTTCTTGCTGAGAAGCTTTCTTCTCGTCCTGAGGCTGTTCATGAGTATGTTACTCCTCTTTTTGTTTCT |
| | | CGTGCTCCTAATCGTAAGATGTCTGGTCAGGGTCATATGGAGACTGTTAAGTCTGCTAAGCGTCTTGATGAGGGTGTTTCTGT |
| | | TCTTCGTGTTCCTCTTACTCAGCTTAAGCTTAAGGATCTTGAGAAGATGGTTAATCGTGAGCGTGAGCCTAAGCTTTATGAGG |
| | | CTCTTAAGGCTCGTCTTGAGGCTCATAAGGATGATCCTGCTAAGGCTTTTGCTGAGCCTTTTTATAAGTATGATAAGGCTGGT |
| | | AATCGTACTCAGCAGGTTAAGGCTGTTCGTGTTGAGCAGGTTCAGAAGACTGGTGTTTGGGTTCGTAATCATAATGGTATTGC |
| | | TGATAATGCTACTATGGTTCGTGTTGATGTTTTTGAGAAGGGTGATAAGTATTATCTTGTTCCTATTTATTCTTGGCAGGTTG |
| | | CTAAGGGTATTCTTCCTGATCGTGCTGTTGTTGCTTATGCTGATGAGGAGGATTGGACTGTTATTGATGAGTCTTTTCGTTTT |
| | | AAGTTTGTTCTTTATTCTAATGATCTTATTAAGGTTCAGCTTAAGAAGGATTCTTTTCTTGGTTATTTTTCTGGTCTTGATCG |
| | | TGCTACTGGTGCTATTTCTCTTCGTGAGCATGATCTTGAGAAGTCTAAGGGTAAGGATGGTATGCATCGTATTGGTGTTAAGA |
| | | CTGCTCTTTCTTTTCAGAAGTATCAGATTGATGAGATGGGTAAGGAGATTCGTCCTTGTCGTCTTAAGAAGCGTCCTCCTGTT |
| | | CGT |
| Exemplary | 299 | GCCGCCTTCAAGCCCAACCCCATCAACTACATCCTGGGCCTGGACATCGGCATCGCCTCCGTGGGCTGGGCCATGGTGGAGAT |
| coding | | CGACGAGGAGGAGAACCCCATCCGGCTGATCGACCTGGGCGTGCGGGTGTTCGAGCGGGCCGAGGTGCCCAAGACCGGCGACT |
| sequence | | CCCTGGCCATGGCCCGGCGGCTGGCCCGGTCCGTGCGGCGGCTGACCCGGCGGCGGGCCCACCGGCTGCTGCGGGCCCGGCGG |
| encoding | | CTGCTGAAGCGGGAGGGCGTGCTGCAGGCCGCCGACTTCGACGAGAACGGCCTGATCAAGTCCCTGCCCAACACCCCCTGGCA |
| Nme3Cas9 | | GCTGCGGGCCGCCGCCCTGGACCGGAAGCTGACCCCCCTGGAGTGGTCCGCCGTGCTGCTGCACCTGATCAAGCACCGGGGCT |
| HNH | | ACCTGTCCCAGCGGAAGAACGAGGGCGAGACCGCCGACAAGGAGCTGGGCGCCCTGCTGAAGGGCGTGGCCGACAACGCCCAC |
| nickase | | GCCCTGCAGACCGGCGACTTCCGGACCCCCGCCGAGCTGGCCCTGAACAAGTTCGAGAAGGAGTGCGGCCACATCCGGAACCA |
| | | GCGGGGCGACTACTCCCACACCTTCTCCCGGAAGGACCTGCAGGCCGAGCTGAACCTGCTGTTCGAGAAGCAGAAGGAGTTCG |
| | | GCAACCCCCACGTGTCCGGCGGCCTGAAGGAGGGCATCGAGACCCTGCTGATGACCCAGCGGCCCGCCCTGTCCGGCGACGCC |
| | | GTGCAGAAGATGCTGGGCCACTGCACCTTCGAGCCCGCCGAGCCCAAGGCCGCCAAGAACACCTACACCGCCGAGCGGTTCAT |
| | | CTGGCTGACCAAGCTGAACAACCTGCGGATCCTGGAGCAGGGCTCCGAGCGGCCCCTGACCGACACCGAGCGGGCCACCCTGA |
| | | TGGACGAGCCCTACCGGAAGTCCAAGCTGACCTACGCCCAGGCCCGGAAGCTGCTGTCCCTGGAGGACACCGCCTTCTTCAAG |
| | | GGCCTGCGGTACGGCAAGGACAACGCCGAGGCCTCCACCCTGATGGAGATGAAGGCCTACCACACCATCTCCCGGGCCCTGGA |
| | | GAAGGAGGGCCTGAAGGACAAGAAGTCCCCCCTGAACCTGTCCCCCGAGCTGCAGGACGAGATCGGCACCGCCTTCTCCCTGT |
| | | TCAAGACCGACGAGGACATCACCGGCCGGCTGAAGGACCGGATCCAGCCCGAGATCCTGGAGGCCCTGCTGAAGCACATCTCC |
| | | TTCGACAAGTTCGTGCAGATCTCCCTGAAGGCCCTGCGGCGGATCGTGCCCCTGATGGAGCAGGGCAAGCGGTACGACGAGGC |
| | | CTGCGCCGAGATCTACGGCGACCACTACGGCAAGAAGAACACCGAGGAGAAGATCTACCTGCCCCCCATCCCCGCCGACGAGA |
| | | TCCGGAACCCCGTGGTGCTGCGGGCCCTGTCCCAGGCCCGGAAGGTGATCAACGGCGTGGTGCGGCGGTACGGCTCCCCCGCC |
| | | CGGATCCACATCGAGACCGCCCGGGAGGTGGGCAAGTCCTTCAAGGACCGGAAGGAGATCGAGAAGCGGCAGGAGGAGAACCG |
| | | GAAGGACCGGGAGAAGGCCGCCGCCAAGTTCCGGGAGTACTTCCCCAACTTCGTGGGCGAGCCCAAGTCCAAGGACATCCTGA |
| | | AGCTGCGGCTGTACGAGCAGCAGCACGGCAAGTGCCTGTACTCCGGCAAGGAGATCAACCTGGGCCGGCTGAACGAGAAGGGC |
| | | TACGTGGAGATCGACGCCGCCCTGCCCTTCTCCCGGACCTGGGACGACTCCTTCAACAACAAGGTGCTGGTGCTGGGCTCCGA |
| | | GAACCAGAACAAGGGCAACCAGACCCCCTACGAGTACTTCAACGGCAAGGACAACTCCCGGGAGTGGCAGGAGTTCAAGGCCC |
| | | GGGTGGAGACCTCCCGGTTCCCCCGGTCCAAGAAGCAGCGGATCCTGCTGCAGAAGTTCGACGAGGACGGCTTCAAGGAGCGG |
| | | AACCTGAACGACACCCGGTACGTGAACCGGTTCCTGTGCCAGTTCGTGGCCGACCGGATGCGGCTGACCGGCAAGGGCAAGAA |
| | | GCGGGTGTTCGCCTCCAACGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACCCGGTTCGTGCGGTACAAG |
| | | GAGATGAACGCCTTCGACGGCAAGACCATCGACAAGGAGACCGGCGAGGTGCTGCACCAGAAGACCCACTTCCCCCAGCCCTG |
| | | GGAGTTCTTCGCCCAGGAGGTGATGATCCGGGTGTTCGGCAAGCCCGACGGCAAGCCCGAGTTCGAGGAGGCCGACACCCCCG |
| | | AGAAGCTGCGGACCCTGCTGGCCGAGAAGCTGTCCTCCCGGCCCGAGGCCGTGCACGAGTACGTGACCCCCCTGTTCGTGTCC |
| | | CGGGCCCCCAACCGGAAGATGTCCGGCCAGGGCCACATGGAGACCGTGAAGTCCGCCAAGCGGCTGGACGAGGGCGTGTCCGT |
| | | GCTGCGGGTGCCCCTGACCCAGCTGAAGCTGAAGGACCTGGAGAAGATGGTGAACCGGGAGCGGGAGCCCAAGCTGTACGAGG |
| | | CCCTGAAGGCCCGGCTGGAGGCCCACAAGGACGACCCCGCCAAGGCCTTCGCCGAGCCCTTCTACAAGTACGACAAGGCCGGC |
| | | AACCGGACCCAGCAGGTGAAGGCCGTGCGGGTGGAGCAGGTGCAGAAGACCGGCGTGTGGGTGCGGAACCACAACGGCATCGC |
| | | CGACAACGCCACCATGGTGCGGGTGGACGTGTTCGAGAAGGGCGACAAGTACTACCTGGTGCCCATCTACTCCTGGCAGGTGG |
| | | CCAAGGGCATCCTGCCCGACCGGGCCGTGGTGGCCTACGCCGACGAGGAGGACTGGACCGTGATCGACGAGTCCTTCCGGTTC |
| | | AAGTTCGTGCTGTACTCCAACGACCTGATCAAGGTGCAGCTGAAGAAGGACTCCTTCCTGGGCTACTTCTCCGGCCTGGACCG |
| | | GGCCACCGGCGCCATCTCCCTGCGGGAGCACGACCTGGAGAAGTCCAAGGGCAAGGACGGCATGCACCGGATCGGCGTGAAGA |
| | | CCGCCCTGTCCTTCCAGAAGTACCAGATCGACGAGATGGGCAAGGAGATCCGGCCCTGCCGGCTGAAGAAGCGGCCCCCCGTG |
| | | CGG |
| Exemplary | 300 | GCAGCATTCAAACCAAACCCAATCAACTACATCCTAGGACTAGACATCGGAATCGCATCAGTAGGATGAGCAATGGTAGAAAT |
| coding | | CGACGAAGAAGAAAACCCAATCCGACTAATCGACCTAGGAGTACGAGTATTCGAACGAGCAGAAGTACCAAAAACAGGAGACT |
| sequence | | CACTAGCAATGGCACGACGACTAGCACGATCAGTACGACGACTAACACGACGACGAGCACACCGACTACTACGAGCACGACGA |
| encoding | | CTACTAAAACGAGAAGGAGTACTACAAGCAGCAGACTTCGACGAAAACGGACTAATCAAATCACTACCAAACACACCATGACA |
| Nme3Cas9 | | ACTACGAGCAGCAGCACTAGACCGAAAACTAACACCACTAGAATGATCAGCAGTACTACTACACCTAATCAAACACCGAGGAT |
| HNH | | ACCTATCACAACGAAAAAACGAAGGAGAAACAGCAGACAAAGAACTAGGAGCACTACTAAAAGGAGTAGCAGACAACGCACAC |
| nickase | | GCACTACAAACAGGAGACTTCCGAACACCAGCAGAACTAGCACTAAACAAATTCGAAAAAGAATGCGGACACATCCGAAACCA |
| | | ACGAGGAGACTACTCACACACATTCTCACGAAAAGACCTACAAGCAGAACTAAACCTACTATTCGAAAAACAAAAAGAATTCG |
| | | GAAACCCACACGTATCAGGAGGACTAAAAGAAGGAATCGAAACACTACTAATGACACAACGACCAGCACTATCAGGAGACGCA |
| | | GTACAAAAAATGCTAGGACACTGCACATTCGAACCAGCAGAACCAAAAGCAGCAAAAAACACATACACAGCAGAACGATTCAT |
| | | CTGACTAACAAAACTAAACAACCTACGAATCCTAGAACAAGGATCAGAACGACCACTAACAGACACAGAACGAGCAACACTAA |
| | | TGGACGAACCATACCGAAAATCAAAACTAACATACGCACAAGCACGAAAACTACTATCACTAGAAGACACAGCATTCTTCAAA |
| | | GGACTACGATACGGAAAAGACAACGCAGAAGCATCAACACTAATGGAAATGAAAGCATACCACACAATCTCACGAGCACTAGA |
| | | AAAAGAAGGACTAAAAGACAAAAAATCACCACTAAACCTATCACCAGAACTACAAGACGAAATCGGAACAGCATTCTCACTAT |
| | | TCAAAACAGACGAAGACATCACAGGACGACTAAAAGACCGAATCCAACCAGAAATCCTAGAAGCACTACTAAAACACATCTCA |
| | | TTCGACAAATTCGTACAAATCTCACTAAAAGCACTACGACGAATCGTACCACTAATGGAACAAGGAAAACGATACGACGAAGC |
| | | ATGCGCAGAAATCTACGGAGACCACTACGGAAAAAAAAACACAGAAGAAAAAATCTACCTACCACCAATCCCAGCAGACGAAA |
| | | TCCGAAACCCAGTAGTACTACGAGCACTATCACAAGCACGAAAAGTAATCAACGGAGTAGTACGACGATACGGATCACCAGCA |
| | | CGAATCCACATCGAAACAGCACGAGAAGTAGGAAAATCATTCAAAGACCGAAAAGAAATCGAAAAACGACAAGAAGAAAACCG |
| | | AAAAGACCGAGAAAAAGCAGCAGCAAAATTCCGAGAATACTTCCCAAACTTCGTAGGAGAACCAAAATCAAAAGACATCCTAA |
| | | AACTACGACTATACGAACAACAACACGGAAAATGCCTATACTCAGGAAAAGAAATCAACCTAGGACGACTAAACGAAAAAGGA |
| | | TACGTAGAAATCGACGCAGCACTACCATTCTCACGAACATGAGACGACTCATTCAACAACAAAGTACTAGTACTAGGATCAGA |
| | | AAACCAAAACAAAGGAAACCAAACACCATACGAATACTTCAACGGAAAAGACAACTCACGAGAATGACAAGAATTCAAAGCAC |
| | | GAGTAGAAACATCACGATTCCCACGATCAAAAAAACAACGAATCCTACTACAAAAATTCGACGAAGACGGATTCAAAGAACGA |
| | | AACCTAAACGACACACGATACGTAAACCGATTCCTATGCCAATTCGTAGCAGACCGAATGCGACTAACAGGAAAAGGAAAAAA |
| | | ACGAGTATTCGCATCAAACGGACAAATCACAAACCTACTACGAGGATTCTGAGGACTACGAAAAGTACGAGCAGAAAACGACC |
| | | GACACCACGCACTAGACGCAGTAGTAGTAGCATGCTCAACAGTAGCAATGCAACAAAAAATCACACGATTCGTACGATACAAA |
| | | GAAATGAACGCATTCGACGGAAAAACAATCGACAAAGAAACAGGAGAAGTACTACACCAAAAAACACACTTCCCACAACCATG |
| | | AGAATTCTTCGCACAAGAAGTAATGATCCGAGTATTCGGAAAACCAGACGGAAAACCAGAATTCGAAGAAGCAGACACACCAG |
| | | AAAAACTACGAACACTACTAGCAGAAAAACTATCATCACGACCAGAAGCAGTACACGAATACGTAACACCACTATTCGTATCA |
| | | CGAGCACCAAACCGAAAAATGTCAGGACAAGGACACATGGAAACAGTAAAATCAGCAAAACGACTAGACGAAGGAGTATCAGT |
| | | ACTACGAGTACCACTAACACAACTAAAACTAAAAGACCTAGAAAAAATGGTAAACCGAGAACGAGAACCAAAACTATACGAAG |
| | | CACTAAAAGCACGACTAGAAGCACACAAAGACGACCCAGCAAAAGCATTCGCAGAACCATTCTACAAATACGACAAAGCAGGA |
| | | AACCGAACACAACAAGTAAAAGCAGTACGAGTAGAACAAGTACAAAAAACAGGAGTATGAGTACGAAACCACAACGGAATCGC |
| | | AGACAACGCAACAATGGTACGAGTAGACGTATTCGAAAAAGGAGACAAATACTACCTAGTACCAATCTACTCATGACAAGTAG |
| | | CAAAAGGAATCCTACCAGACCGAGCAGTAGTAGCATACGCAGACGAAGAAGACTGAACAGTAATCGACGAATCATTCCGATTC |
| | | AAATTCGTACTATACTCAAACGACCTAATCAAAGTACAACTAAAAAAAGACTCATTCCTAGGATACTTCTCAGGACTAGACCG |
| | | AGCAACAGGAGCAATCTCACTACGAGAACACGACCTAGAAAAATCAAAAGGAAAAGACGGAATGCACCGAATCGGAGTAAAAA |
| | | CAGCACTATCATTCCAAAAATACCAAATCGACGAAATGGGAAAAGAAATCCGACCATGCCGACTAAAAAAACGACCACCAGTA |
| | | CGA |
| Exemplary | 301 | ATGGCTGCTTTTAAGCCTAATCCTATTAATTATATTCTTGGTCTTGATATTGGTATTGCTTCTGTTGGTTGGGCTATGGTTGA |
| open | | GATTGATGAGGAGGAGAATCCTATTCGTCTTATTGATCTTGGTGTTCGTGTTTTTGAGCGTGCTGAGGTTCCTAAGACTGGTG |
| reading | | ATTCTCTTGCTATGGCTCGTCGTCTTGCTCGTTCTGTTCGTCGTCTTACTCGTCGTCGTGCTCATCGTCTTCTTCGTGCTCGT |
| frame for | | CGTCTTCTTAAGCGTGAGGGTGTTCTTCAGGCTGCTGATTTTGATGAGAATGGTCTTATTAAGTCTCTTCCTAATACTCCTTG |
| Nme3Cas9 | | GCAGCTTCGTGCTGCTGCTCTTGATCGTAAGCTTACTCCTCTTGAGTGGTCTGCTGTTCTTCTTCATCTTATTAAGCATCGTG |
| HNH | | GTTATCTTTCTCAGCGTAAGAATGAGGGTGAGACTGCTGATAAGGAGCTTGGTGCTCTTCTTAAGGGTGTTGCTGATAATGCT |
| nickase | | CATGCTCTTCAGACTGGTGATTTTCGTACTCCTGCTGAGCTTGCTCTTAATAAGTTTGAGAAGGAGTGTGGTCATATTCGTAA |
| | | TCAGCGTGGTGATTATTCTCATACTTTTTCTCGTAAGGATCTTCAGGCTGAGCTTAATCTTCTTTTTGAGAAGCAGAAGGAGT |
| | | TTGGTAATCCTCATGTTTCTGGTGGTCTTAAGGAGGGTATTGAGACTCTTCTTATGACTCAGCGTCCTGCTCTTTCTGGTGAT |
| | | GCTGTTCAGAAGATGCTTGGTCATTGTACTTTTGAGCCTGCTGAGCCTAAGGCTGCTAAGAATACTTATACTGCTGAGCGTTT |
| | | TATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTTCTCTTGAGGATACTGCTTTTTTT |
| | | AAGGGTCTTCGTTATGGTAAGGATAATGCTGAGGCTTCTACTCTTATGGAGATGAAGGCTTATCATACTATTTCTCGTGCTCT |
| | | TGAGAAGGAGGGTCTTAAGGATAAGAAGTCTCCTCTTAATCTTTCTCCTGAGCTTCAGGATGAGATTGGTACTGCTTTTTCTC |
| | | TTTTTAAGACTGATGAGGATATTACTGGTCGTCTTAAGGATCGTATTCAGCCTGAGATTCTTGAGGCTCTTCTTAAGCATATT |
| | | TCTTTTGATAAGTTTGTTCAGATTTCTCTTAAGGCTCTTCGTCGTATTGTTCCTCTTATGGAGCAGGGTAAGCGTTATGATGA |
| | | GGCTTGTGCTGAGATTTATGGTGATCATTATGGTAAGAAGAATACTGAGGAGAAGATTTATCTTCCTCCTATTCCTGCTGATG |
| | | AGATTCGTAATCCTGTTGTTCTTCGTGCTCTTTCTCAGGCTCGTAAGGTTATTAATGGTGTTGTTCGTCGTTATGGTTCTCCT |
| | | GCTCGTATTCATATTGAGACTGCTCGTGAGGTTGGTAAGTCTTTTAAGGATCGTAAGGAGATTGAGAAGCGTCAGGAGGAGAA |
| | | TCGTAAGGATCGTGAGAAGGCTGCTGCTAAGTTTCGTGAGTATTTTCCTAATTTTGTTGGTGAGCCTAAGTCTAAGGATATTC |
| | | TTAAGCTTCGTCTTTATGAGCAGCAGCATGGTAAGTGTCTTTATTCTGGTAAGGAGATTAATCTTGGTCGTCTTAATGAGAAG |
| | | GGTTATGTTGAGATTGATGCTGCTCTTCCTTTTTCTCGTACTTGGGATGATTCTTTTAATAATAAGGTTCTTGTTCTTGGTTC |
| | | TGAGAATCAGAATAAGGGTAATCAGACTCCTTATGAGTATTTTAATGGTAAGGATAATTCTCGTGAGTGGCAGGAGTTTAAGG |
| | | CTCGTGTTGAGACTTCTCGTTTTCCTCGTTCTAAGAAGCAGCGTATTCTTCTTCAGAAGTTTGATGAGGATGGTTTTAAGGAG |
| | | CGTAATCTTAATGATACTCGTTATGTTAATCGTTTTCTTTGTCAGTTTGTTGCTGATCGTATGCGTCTTACTGGTAAGGGTAA |
| | | GAAGCGTGTTTTTGCTTCTAATGGTCAGATTACTAATCTTCTTCGTGGTTTTTGGGGTCTTCGTAAGGTTCGTGCTGAGAATG |
| | | ATCGTCATCATGCTCTTGATGCTGTTGTTGTTGCTTGTTCTACTGTTGCTATGCAGCAGAAGATTACTCGTTTTGTTCGTTAT |
| | | AAGGAGATGAATGCTTTTGATGGTAAGACTATTGATAAGGAGACTGGTGAGGTTCTTCATCAGAAGACTCATTTTCCTCAGCC |
| | | TTGGGAGTTTTTTGCTCAGGAGGTTATGATTCGTGTTTTTGGTAAGCCTGATGGTAAGCCTGAGTTTGAGGAGGCTGATACTC |
| | | CTGAGAAGCTTCGTACTCTTCTTGCTGAGAAGCTTTCTTCTCGTCCTGAGGCTGTTCATGAGTATGTTACTCCTCTTTTTGTT |
| | | TCTCGTGCTCCTAATCGTAAGATGTCTGGTCAGGGTCATATGGAGACTGTTAAGTCTGCTAAGCGTCTTGATGAGGGTGTTTC |
| | | TGTTCTTCGTGTTCCTCTTACTCAGCTTAAGCTTAAGGATCTTGAGAAGATGGTTAATCGTGAGCGTGAGCCTAAGCTTTATG |
| | | AGGCTCTTAAGGCTCGTCTTGAGGCTCATAAGGATGATCCTGCTAAGGCTTTTGCTGAGCCTTTTTATAAGTATGATAAGGCT |
| | | GGTAATCGTACTCAGCAGGTTAAGGCTGTTCGTGTTGAGCAGGTTCAGAAGACTGGTGTTTGGGTTCGTAATCATAATGGTAT |
| | | TGCTGATAATGCTACTATGGTTCGTGTTGATGTTTTTGAGAAGGGTGATAAGTATTATCTTGTTCCTATTTATTCTTGGCAGG |
| | | TTGCTAAGGGTATTCTTCCTGATCGTGCTGTTGTTGCTTATGCTGATGAGGAGGATTGGACTGTTATTGATGAGTCTTTTCGT |
| | | TTTAAGTTTGTTCTTTATTCTAATGATCTTATTAAGGTTCAGCTTAAGAAGGATTCTTTTCTTGGTTATTTTTCTGGTCTTGA |
| | | TCGTGCTACTGGTGCTATTTCTCTTCGTGAGCATGATCTTGAGAAGTCTAAGGGTAAGGATGGTATGCATCGTATTGGTGTTA |
| | | AGACTGCTCTTTCTTTTCAGAAGTATCAGATTGATGAGATGGGTAAGGAGATTCGTCCTTGTCGTCTTAAGAAGCGTCCTCCT |
| | | GTTCGTUGA |
| Exemplary | 302 | ATGGCCGCCTTCAAGCCCAACCCCATCAACTACATCCTGGGCCTGGACATCGGCATCGCCTCCGTGGGCTGGGCCATGGTGGA |
| open | | GATCGACGAGGAGGAGAACCCCATCCGGCTGATCGACCTGGGCGTGCGGGTGTTCGAGCGGGCCGAGGTGCCCAAGACCGGCG |
| reading | | ACTCCCTGGCCATGGCCCGGCGGCTGGCCCGGTCCGTGCGGCGGCTGACCCGGCGGCGGGCCCACCGGCTGCTGCGGGCCCGG |
| frame for | | CGGCTGCTGAAGCGGGAGGGCGTGCTGCAGGCCGCCGACTTCGACGAGAACGGCCTGATCAAGTCCCTGCCCAACACCCCCTG |
| Nme3Cas9 | | GCAGCTGCGGGCCGCCGCCCTGGACCGGAAGCTGACCCCCCTGGAGTGGTCCGCCGTGCTGCTGCACCTGATCAAGCACCGGG |
| HNH | | GCTACCTGTCCCAGCGGAAGAACGAGGGCGAGACCGCCGACAAGGAGCTGGGCGCCCTGCTGAAGGGCGTGGCCGACAACGCC |
| nickase | | CACGCCCTGCAGACCGGCGACTTCCGGACCCCCGCCGAGCTGGCCCTGAACAAGTTCGAGAAGGAGTGCGGCCACATCCGGAA |
| | | CCAGCGGGGCGACTACTCCCACACCTTCTCCCGGAAGGACCTGCAGGCCGAGCTGAACCTGCTGTTCGAGAAGCAGAAGGAGT |
| | | TCGGCAACCCCCACGTGTCCGGCGGCCTGAAGGAGGGCATCGAGACCCTGCTGATGACCCAGCGGCCCGCCCTGTCCGGCGAC |
| | | GCCGTGCAGAAGATGCTGGGCCACTGCACCTTCGAGCCCGCCGAGCCCAAGGCCGCCAAGAACACCTACACCGCCGAGCGGTT |
| | | CATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGTCCCTGGAGGACACCGCCTTCTTC |
| | | AAGGGCCTGCGGTACGGCAAGGACAACGCCGAGGCCTCCACCCTGATGGAGATGAAGGCCTACCACACCATCTCCCGGGCCCT |
| | | GGAGAAGGAGGGCCTGAAGGACAAGAAGTCCCCCCTGAACCTGTCCCCCGAGCTGCAGGACGAGATCGGCACCGCCTTCTCCC |
| | | TGTTCAAGACCGACGAGGACATCACCGGCCGGCTGAAGGACCGGATCCAGCCCGAGATCCTGGAGGCCCTGCTGAAGCACATC |
| | | TCCTTCGACAAGTTCGTGCAGATCTCCCTGAAGGCCCTGCGGCGGATCGTGCCCCTGATGGAGCAGGGCAAGCGGTACGACGA |
| | | GGCCTGCGCCGAGATCTACGGCGACCACTACGGCAAGAAGAACACCGAGGAGAAGATCTACCTGCCCCCCATCCCCGCCGACG |
| | | AGATCCGGAACCCCGTGGTGCTGCGGGCCCTGTCCCAGGCCCGGAAGGTGATCAACGGCGTGGTGCGGCGGTACGGCTCCCCC |
| | | GCCCGGATCCACATCGAGACCGCCCGGGAGGTGGGCAAGTCCTTCAAGGACCGGAAGGAGATCGAGAAGCGGCAGGAGGAGAA |
| | | CCGGAAGGACCGGGAGAAGGCCGCCGCCAAGTTCCGGGAGTACTTCCCCAACTTCGTGGGCGAGCCCAAGTCCAAGGACATCC |
| | | TGAAGCTGCGGCTGTACGAGCAGCAGCACGGCAAGTGCCTGTACTCCGGCAAGGAGATCAACCTGGGCCGGCTGAACGAGAAG |
| | | GGCTACGTGGAGATCGACGCCGCCCTGCCCTTCTCCCGGACCTGGGACGACTCCTTCAACAACAAGGTGCTGGTGCTGGGCTC |
| | | CGAGAACCAGAACAAGGGCAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGACGAGGACGGCTTCAAGGAG |
| | | CGGAACCTGAACGACACCCGGTACGTGAACCGGTTCCTGTGCCAGTTCGTGGCCGACCGGATGCGGCTGACCGGCAAGGGCAA |
| | | GAAGCGGGTGTTCGCCTCCAACGGCCAGATCACCAACCTGCTGCGGGGCTTCTGGGGCCTGCGGAAGGTGCGGGCCGAGAACG |
| | | ACCGGCACCACGCCCTGGACGCCGTGGTGGTGGCCTGCTCCACCGTGGCCATGCAGCAGAAGATCACCCGGTTCGTGCGGTAC |
| | | AAGGAGATGAACGCCTTCGACGGCAAGACCATCGACAAGGAGACCGGCGAGGTGCTGCACCAGAAGACCCACTTCCCCCAGCC |
| | | CTGGGAGTTCTTCGCCCAGGAGGTGATGATCCGGGTGTTCGGCAAGCCCGACGGCAAGCCCGAGTTCGAGGAGGCCGACACCC |
| | | CCGAGAAGCTGCGGACCCTGCTGGCCGAGAAGCTGTCCTCCCGGCCCGAGGCCGTGCACGAGTACGTGACCCCCCTGTTCGTG |
| | | TCCCGGGCCCCCAACCGGAAGATGTCCGGCCAGGGCCACATGGAGACCGTGAAGTCCGCCAAGCGGCTGGACGAGGGCGTGTC |
| | | CGTGCTGCGGGTGCCCCTGACCCAGCTGAAGCTGAAGGACCTGGAGAAGATGGTGAACCGGGAGCGGGAGCCCAAGCTGTACG |
| | | AGGCCCTGAAGGCCCGGCTGGAGGCCCACAAGGACGACCCCGCCAAGGCCTTCGCCGAGCCCTTCTACAAGTACGACAAGGCC |
| | | GGCAACCGGACCCAGCAGGTGAAGGCCGTGCGGGTGGAGCAGGTGCAGAAGACCGGCGTGTGGGTGCGGAACCACAACGGCAT |
| | | CGCCGACAACGCCACCATGGTGCGGGTGGACGTGTTCGAGAAGGGCGACAAGTACTACCTGGTGCCCATCTACTCCTGGCAGG |
| | | TGGCCAAGGGCATCCTGCCCGACCGGGCCGTGGTGGCCTACGCCGACGAGGAGGACTGGACCGTGATCGACGAGTCCTTCCGG |
| | | TTCAAGTTCGTGCTGTACTCCAACGACCTGATCAAGGTGCAGCTGAAGAAGGACTCCTTCCTGGGCTACTTCTCCGGCCTGGA |
| | | CCGGGCCACCGGCGCCATCTCCCTGCGGGAGCACGACCTGGAGAAGTCCAAGGGCAAGGACGGCATGCACCGGATCGGCGTGA |
| | | AGACCGCCCTGTCCTTCCAGAAGTACCAGATCGACGAGATGGGCAAGGAGATCCGGCCCTGCCGGCTGAAGAAGCGGCCCCCC |
| | | GTGCGGUGA |
| Exemplary | 303 | ATGGCAGCATTCAAACCAAACCCAATCAACTACATCCTAGGACTAGACATCGGAATCGCATCAGTAGGATGAGCAATGGTAGA |
| open | | AATCGACGAAGAAGAAAACCCAATCCGACTAATCGACCTAGGAGTACGAGTATTCGAACGAGCAGAAGTACCAAAAACAGGAG |
| reading | | ACTCACTAGCAATGGCACGACGACTAGCACGATCAGTACGACGACTAACACGACGACGAGCACACCGACTACTACGAGCACGA |
| frame for | | CGACTACTAAAACGAGAAGGAGTACTACAAGCAGCAGACTTCGACGAAAACGGACTAATCAAATCACTACCAAACACACCATG |
| Nme3Cas9 | | ACAACTACGAGCAGCAGCACTAGACCGAAAACTAACACCACTAGAATGATCAGCAGTACTACTACACCTAATCAAACACCGAG |
| HNH | | GATACCTATCACAACGAAAAAACGAAGGAGAAACAGCAGACAAAGAACTAGGAGCACTACTAAAAGGAGTAGCAGACAACGCA |
| nickase | | CACGCACTACAAACAGGAGACTTCCGAACACCAGCAGAACTAGCACTAAACAAATTCGAAAAAGAATGCGGACACATCCGAAA |
| | | CCAACGAGGAGACTACTCACACACATTCTCACGAAAAGACCTACAAGCAGAACTAAACCTACTATTCGAAAAACAAAAAGAAT |
| | | TCGGAAACCCACACGTATCAGGAGGACTAAAAGAAGGAATCGAAACACTACTAATGACACAACGACCAGCACTATCAGGAGAC |
| | | GCAGTACAAAAAATGCTAGGACACTGCACATTCGAACCAGCAGAACCAAAAGCAGCAAAAAACACATACACAGCAGAACGATT |
| | | CATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTATCACTAGAAGACACAGCATTCTTC |
| | | AAAGGACTACGATACGGAAAAGACAACGCAGAAGCATCAACACTAATGGAAATGAAAGCATACCACACAATCTCACGAGCACT |
| | | AGAAAAAGAAGGACTAAAAGACAAAAAATCACCACTAAACCTATCACCAGAACTACAAGACGAAATCGGAACAGCATTCTCAC |
| | | TATTCAAAACAGACGAAGACATCACAGGACGACTAAAAGACCGAATCCAACCAGAAATCCTAGAAGCACTACTAAAACACATC |
| | | TCATTCGACAAATTCGTACAAATCTCACTAAAAGCACTACGACGAATCGTACCACTAATGGAACAAGGAAAACGATACGACGA |
| | | AGCATGCGCAGAAATCTACGGAGACCACTACGGAAAAAAAAACACAGAAGAAAAAATCTACCTACCACCAATCCCAGCAGACG |
| | | AAATCCGAAACCCAGTAGTACTACGAGCACTATCACAAGCACGAAAAGTAATCAACGGAGTAGTACGACGATACGGATCACCA |
| | | GCACGAATCCACATCGAAACAGCACGAGAAGTAGGAAAATCATTCAAAGACCGAAAAGAAATCGAAAAACGACAAGAAGAAAA |
| | | CCGAAAAGACCGAGAAAAAGCAGCAGCAAAATTCCGAGAATACTTCCCAAACTTCGTAGGAGAACCAAAATCAAAAGACATCC |
| | | TAAAACTACGACTATACGAACAACAACACGGAAAATGCCTATACTCAGGAAAAGAAATCAACCTAGGACGACTAAACGAAAAA |
| | | GGATACGTAGAAATCGACGCAGCACTACCATTCTCACGAACATGAGACGACTCATTCAACAACAAAGTACTAGTACTAGGATC |
| | | AGAAAACCAAAACAAAGGAAACCAAACACCATACGAATACTTCAACGGAAAAGACAACTCACGAGAATGACAAGAATTCAAAG |
| | | CACGAGTAGAAACATCACGATTCCCACGATCAAAAAAACAACGAATCCTACTACAAAAATTCGACGAAGACGGATTCAAAGAA |
| | | CGAAACCTAAACGACACACGATACGTAAACCGATTCCTATGCCAATTCGTAGCAGACCGAATGCGACTAACAGGAAAAGGAAA |
| | | AAAACGAGTATTCGCATCAAACGGACAAATCACAAACCTACTACGAGGATTCTGAGGACTACGAAAAGTACGAGCAGAAAACG |
| | | ACCGACACCACGCACTAGACGCAGTAGTAGTAGCATGCTCAACAGTAGCAATGCAACAAAAAATCACACGATTCGTACGATAC |
| | | AAAGAAATGAACGCATTCGACGGAAAAACAATCGACAAAGAAACAGGAGAAGTACTACACCAAAAAACACACTTCCCACAACC |
| | | ATGAGAATTCTTCGCACAAGAAGTAATGATCCGAGTATTCGGAAAACCAGACGGAAAACCAGAATTCGAAGAAGCAGACACAC |
| | | CAGAAAAACTACGAACACTACTAGCAGAAAAACTATCATCACGACCAGAAGCAGTACACGAATACGTAACACCACTATTCGTA |
| | | TCACGAGCACCAAACCGAAAAATGTCAGGACAAGGACACATGGAAACAGTAAAATCAGCAAAACGACTAGACGAAGGAGTATC |
| | | AGTACTACGAGTACCACTAACACAACTAAAACTAAAAGACCTAGAAAAAATGGTAAACCGAGAACGAGAACCAAAACTATACG |
| | | AAGCACTAAAAGCACGACTAGAAGCACACAAAGACGACCCAGCAAAAGCATTCGCAGAACCATTCTACAAATACGACAAAGCA |
| | | GGAAACCGAACACAACAAGTAAAAGCAGTACGAGTAGAACAAGTACAAAAAACAGGAGTATGAGTACGAAACCACAACGGAAT |
| | | CGCAGACAACGCAACAATGGTACGAGTAGACGTATTCGAAAAAGGAGACAAATACTACCTAGTACCAATCTACTCATGACAAG |
| | | TAGCAAAAGGAATCCTACCAGACCGAGCAGTAGTAGCATACGCAGACGAAGAAGACTGAACAGTAATCGACGAATCATTCCGA |
| | | TTCAAATTCGTACTATACTCAAACGACCTAATCAAAGTACAACTAAAAAAAGACTCATTCCTAGGATACTTCTCAGGACTAGA |
| | | CCGAGCAACAGGAGCAATCTCACTACGAGAACACGACCTAGAAAAATCAAAAGGAAAAGACGGAATGCACCGAATCGGAGTAA |
| | | AAACAGCACTATCATTCCAAAAATACCAAATCGACGAAATGGGAAAAGAAATCCGACCATGCCGACTAAAAAAACGACCACCA |
| | | GTACGAUAA |
| Exemplary | 304 | GGGAAGCUCAGAAUAAACGCUCAACUUUGGCCGGAUCUGCCACCAUGGACGGCUCCGGCGGCGGCUCCCCCAAGAAGAAGCGG |
| coding | | AAGGUGGAGGACAAGCGGCCCGCCGCCACCAAGAAGGCCGGCCAGGCCAAGAAGAAGAAGGGCGGCUCCGGCGGCGGCGCCGC |
| sequence | | CUUCAAGCCCAACCCCAUCAACUACAUCCUGGGCCUGGACAUCGGCAUCGCCUCCGUGGGCUGGGCCAUGGUGGAGAUCGACG |
| encoding | | AGGAGGAGAACCCCAUCCGGCUGAUCGACCUGGGCGUGCGGGUGUUCGAGCGGGCCGAGGUGCCCAAGACCGGCGACUCCCUG |
| Nme2Cas9 | | GCCAUGGCCCGGCGGCUGGCCCGGUCCGUGCGGCGGCUGACCCGGCGGCGGGCCCACCGGCUGCUGCGGGCCCGGCGGCUGCU |
| | | GAAGCGGGAGGGCGUGCUGCAGGCCGCCGACUUCGACGAGAACGGCCUGAUCAAGUCCCUGCCCAACACCCCCUGGCAGCUGC |
| | | GGGCCGCCGCCCUGGACCGGAAGCUGACCCCCCUGGAGUGGUCCGCCGUGCUGCUGCACCUGAUCAAGCACCGGGGCUACCUG |
| | | UCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGG |
| | | GCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGAC |
| | | GAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGG |
| | | AGGGCCUGAAGGACAAGAAGUCCCCCCUGAACCUGUCCUCCGAGCUGCAGGACGAGAUCGGCACCGCCUUCUCCCUGUUCAAG |
| | | ACCGACGAGGACAUCACCGGCCGGCUGAAGGACCGGGUGCAGCCCGAGAUCCUGGAGGCCCUGCUGAAGCACAUCUCCUUCGA |
| | | CAAGUUCGUGCAGAUCUCCCUGAAGGCCCUGCGGCGGAUCGUGCCCCUGAUGGAGCAGGGCAAGCGGUACGACGAGGCCUGCG |
| | | CCGAGAUCUACGGCGACCACUACGGCAAGAAGAACACCGAGGAGAAGAUCUACCUGCCCCCCAUCCCCGCCGACGAGAUCCGG |
| | | AACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGG |
| | | ACCGGGAGAAGGCCGCCGCCAAGUUCCGGGAGUACUUCCCCAACUUCGUGGGCGAGCCCAAGUCCAAGGACAUCCUGAAGCUG |
| | | CGGCUGUACGAGCAGCAGCACGGCAAGUGCCUGUACUCCGGCAAGGAGAUCAACCUGGUGCGGCUGAACGAGAAGGGCUACGU |
| | | GGAGAUCGACCACGCCCUGCCCUUCUCCCGGACCUGGGACGACUCCUUCAACAACAAGGUGCUGGUGCUGGGCUCCGAGAACC |
| | | AGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGUGCAACCU |
| | | GAACGACACCCGGUACGUGAACCGGUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAU |
| | | GAACGCCUUCGACGGCAAGACCAUCGACAAGGAGACCGGCAAGGUGCUGCACCAGAAGACCCACUUCCCCCAGCCCUGGGAGU |
| | | UCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCG |
| | | UGAAGCGGGUGUGGCUGACCGAGAUCAAGCUGGCCGACCUGGAGAACAUGGUGAACUACAAGAACGGCCGGGAGAUCGAGCUG |
| | | UACGAGGCCCUGAAGGCCCGGCUGGAGGCCUACGGCGGCAACGCCAAGCAGGCCUUCGACCCCAAGGACAACCCCUUCUACAA |
| | | GAAGGGCGGCCAGCUGGUGAAGGCCGUGCGGGUGGAGAAGACCCAGGAGUCCGGCGUGCUGCUGAACAAGAAGAACGCCUACA |
| | | CCAUCGCCGACAACGGCGACAUGGUGCGGGUGGACGUGUUCUGCAAGGUGGACAAGAAGGGCAAGAACCAGUACUUCAUCGUG |
| | | CCCAUCUACGCCUGGCAGGUGGCCGAGAACAUCCUGCCCGACAUCGACUGCAAGGGCUACCGGAUCGACGACUCCUACACCUU |
| | | CUGCUUCUCCCUGCACAAGUACGACCUGAUCGCCUUCCAGAAGGACGAGAAGUCCAAGGUGGAGUUCGCCUACUACAUCAACU |
| | | GCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCG |
| | | GUAGCUAGCACCAGCCUCAAGAACACCCGAAUGGAGUCUCUAAGCUACAUAAUACCAACUUACACUUUACAAAAUGUUGUCCC |
| | | CCAAAAUGUAGCCAUUCGUAUCUGCUCCUAAUAAAAAGAAAGUUUCUUCACAUUCUCAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAA |
| Exemplary | 305 | GGGUCCCGCAGUCGGCGUCCAGCGGCUCUGCUUGUUCGUGUGUGUGUCGUUGCAGGCCUUAUUCGGAUCCGCCACCAUGGCAG |
| coding | | CAUUCAAGCCGAACUCGAUCAACUACAUCCUGGGACUGGACAUCGGAAUCGCAUCGGUCGGAUGGGCAAUGGUCGAAAUCGAC |
| sequence | | GAAGAAGAAAACCCGAUCAGACUGAUCGACCUGGGAGUCAGAGUCUUCGAAAGAGCAGAAGUCCCGAAGACAGGAGACUCGCU |
| encoding | | GGCAAUGGCAAGAAGACUGGCAAGAUCGGUCAGAAGACUGACAAGAAGAAGAGCACACAGACUGCUGAGAACAAGAAGACUGC |
| Nme1Cas9 | | UGAAGAGAGAAGGAGUCCUGCAGGCAGCAAACUUCGACGAAAACGGACUGAUCAAGUCGCUGCCGAACACACCGUGGCAGCUG |
| | | AGAGCAGCAGCACUGGACAGAAAGCUGACACCGCUGGAAUGGUCGGCAGUCCUGCUGCACCUGAUCAAGCACAGAGGAUACCU |
| | | GUCGCAGAGAAAGAACGAAGGAGAAACAGCAGACAAGGAACUGGGAGCACUGCUGAAGGGAGUCGCAGGAAACGCACACGCAC |
| | | UGCAGACAGGAGACUUCAGAACACCGGCAGAACUGGCACUGAACAAGUUCGAAAAGGAAUCGGGACACAUCAGAAACCAGAGA |
| | | UCGGACUACUCGCACACAUUCUCGAGAAAGGACCUGCAGGCAGAACUGAUCCUGCUGUUCGAAAAGCAGAAGGAAUUCGGAAA |
| | | CCCGCACGUCUCGGGAGGACUGAAGGAAGGAAUCGAAACACUGCUGAUGACACAGAGACCGGCACUGUCGGGAGACGCAGUCC |
| | | AGAAGAUGCUGGGACACUGCACAUUCGAACCGGCAGAACCGAAGGCAGCAAAGAACACAUACACAGCAGAAAGAUUCAUCUGG |
| | | CUGACAAAGCUGAACAACCUGAGAAUCCUGGAACAGGGAUCGGAAAGACCGCUGACAGACACAGAAAGAGCAACACUGAUGGA |
| | | CGAACCGUACAGAAAGUCGAAGCUGACAUACGCACAGGCAAGAAAGCUGCUGGGACUGGAAGACACAGCAUUCUUCAAGGGAC |
| | | UGAGAUACGGAAAGGACAACGCAGAAGCAUCGACACUGAUGGAAAUGAAGGCAUACCACGCAAUCUCGAGAGCACUGGAAAAG |
| | | GAAGGACUGAAGGACAAGAAGUCGCCGCUGAACCUGUCGCCGGAACUGCAGGACGAAAUCGGAACAGCAUUCUCGCUGUUCAA |
| | | GACAGACGAAGACAUCACAGGAAGACUGAAGGACAGAAUCCAGCCGGAAAUCCUGGAAGCACUGCUGAAGCACAUCUCGUUCG |
| | | ACAAGUUCGUCCAGAUCUCGCUGAAGGCACUGAGAAGAAUCGUCCCGCUGAUGGAACAGGGAAAGAGAUACGACGAAGCAUGC |
| | | GCAGAAAUCUACGGAGACCACUACGGAAAGAAGAACACAGAAGAAAAGAUCUACCUGCCGCCGAUCCCGGCAGACGAAAUCAG |
| | | AAACCCGGUCGUCCUGAGAGCACUGUCGCAGGCAAGAAAGGUCAUCAACGGAGUCGUCAGAAGAUACGGAUCGCCGGCAAGAA |
| | | UCCACAUCGAAACAGCAAGAGAAGUCGGAAAGUCGUUCAAGGACAGAAAGGAAAUCGAAAAGAGACAGGAAGAAAACAGAAAG |
| | | GACAGAGAAAAGGCAGCAGCAAAGUUCAGAGAAUACUUCCCGAACUUCGUCGGAGAACCGAAGUCGAAGGACAUCCUGAAGCU |
| | | GAGACUGUACGAACAGCAGCACGGAAAGUGCCUGUACUCGGGAAAGGAAAUCAACCUGGGAAGACUGAACGAAAAGGGAUACG |
| | | UCGAAAUCGACCACGCACUGCCGUUCUCGAGAACAUGGGACGACUCGUUCAACAACAAGGUCCUGGUCCUGGGAUCGGAAAAC |
| | | CAGAACAAGGGAAACCAGACACCGUACGAAUACUUCAACGGAAAGGACAACUCGAGAGAAUGGCAGGAAUUCAAGGCAAGAGU |
| | | CGAAACAUCGAGAUUCCCGAGAUCGAAGAAGCAGAGAAUCCUGCUGCAGAAGUUCGACGAAGACGGAUUCAAGGAAAGAAACC |
| | | UGAACGACACAAGAUACGUCAACAGAUUCCUGUGCCAGUUCGUCGCAGACAGAAUGAGACUGACAGGAAAGGGAAAGAAGAGA |
| | | GUCUUCGCAUCGAACGGACAGAUCACAAACCUGCUGAGAGGAUUCUGGGGACUGAGAAAGGUCAGAGCAGAAAACGACAGACA |
| | | CCACGCACUGGACGCAGUCGUCGUCGCAUGCUCGACAGUCGCAAUGCAGCAGAAGAUCACAAGAUUCGUCAGAUACAAGGAAA |
| | | UGAACGCAUUCGACGGAAAGACAAUCGACAAGGAAACAGGAGAAGUCCUGCACCAGAAGACACACUUCCCGCAGCCGUGGGAA |
| | | UUCUUCGCACAGGAAGUCAUGAUCAGAGUCUUCGGAAAGCCGGACGGAAAGCCGGAAUUCGAAGAAGCAGACACACUGGAAAA |
| | | GCUGAGAACACUGCUGGCAGAAAAGCUGUCGUCGAGACCGGAAGCAGUCCACGAAUACGUCACACCGCUGUUCGUCUCGAGAG |
| | | CACCGAACAGAAAGAUGUCGGGACAGGGACACAUGGAAACAGUCAAGUCGGCAAAGAGACUGGACGAAGGAGUCUCGGUCCUG |
| | | AGAGUCCCGCUGACACAGCUGAAGCUGAAGGACCUGGAAAAGAUGGUCAACAGAGAAAGAGAACCGAAGCUGUACGAAGCACU |
| | | GAAGGCAAGACUGGAAGCACACAAGGACGACCCGGCAAAGGCAUUCGCAGAACCGUUCUACAAGUACGACAAGGCAGGAAACA |
| | | GAACACAGCAGGUCAAGGCAGUCAGAGUCGAACAGGUCCAGAAGACAGGAGUCUGGGUCAGAAACCACAACGGAAUCGCAGAC |
| | | AACGCAACAAUGGUCAGAGUAGACGUCUUCGAAAAGGGAGACAAGUACUACCUGGUCCCGAUCUACUCGUGGCAGGUCGCAAA |
| | | GGGAAUCCUGCCGGACAGAGCAGUCGUCCAGGGAAAGGACGAAGAAGACUGGCAGCUGAUCGACGACUCGUUCAACUUCAAGU |
| | | UCUCGCUGCACCCGAACGACCUGGUCGAAGUCAUCACAAAGAAGGCAAGAAUGUUCGGAUACUUCGCAUCGUGCCACAGAGGA |
| | | ACAGGAAACAUCAACAUCAGAAUCCACGACCUGGACCACAAGAUCGGAAAGAACGGAAUCCUGGAAGGAAUCGGAGUCAAGAC |
| | | AGCACUGUCGUUCCAGAAGUACCAGAUCGACGAACUGGGAAAGGAAAUCAGACCGUGCAGACUGAAGAAGAGACCGCCGGUCA |
| | | GAUCCGGAAAGAGAACAGCAGACGGAUCGGAAUUCGAAUCGCCGAAGAAGAAGAGAAAGGUCGAAUGAUAGCUAGCCAUCACA |
| | | UUUAAAAGCAUCUCAGCCUACCAUGAGAAUAAGAGAAAGAAAAUGAAGAUCAAUAGCUUAUUCAUCUCUUUUUCUUUUUCGUU |
| | | GGUGUAAAGCCAACACCCUGUCUAAAAAACAUAAAUUUCUUUAAUCAUUUUGCCUCUUUUCUCUGUGCUUCAAUUAAUAAAAA |
| | | AUGGAAAGAACCUCGAGAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAUCUAG |
| Exemplary | 306 | GGGAAGCUCAGAAUAAACGCUCAACUUUGGCCGGAUCUGCCACCAUGGACGGCUCCGGCGGCGGCUCCCCCAAGAAGAAGCGG |
| coding | | AAGGUGGAGGACAAGCGGCCCGCCGCCACCAAGAAGGCCGGCCAGGCCAAGAAGAAGAAGGGCGGCUCCGGCGGCGGCGCCGC |
| sequence | | CUUCAAGCCCAACCCCAUCAACUACAUCCUGGGCCUGGACAUCGGCAUCGCCUCCGUGGGCUGGGCCAUGGUGGAGAUCGACG |
| encoding | | AGGAGGAGAACCCCAUCCGGCUGAUCGACCUGGGCGUGCGGGUGUUCGAGCGGGCCGAGGUGCCCAAGACCGGCGACUCCCUG |
| Nme2Cas9 | | GCCAUGGCCCGGCGGCUGGCCCGGUCCGUGCGGCGGCUGACCCGGCGGCGGGCCCACCGGCUGCUGCGGGCCCGGCGGCUGCU |
| with | | GAAGCGGGAGGGCGUGCUGCAGGCCGCCGACUUCGACGAGAACGGCCUGAUCAAGUCCCUGCCCAACACCCCCUGGCAGCUGC |
| HiBiT tag | | GGGCCGCCGCCCUGGACCGGAAGCUGACCCCCCUGGAGUGGUCCGCCGUGCUGCUGCACCUGAUCAAGCACCGGGGCUACCUG |
| | | UCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGG |
| | | GCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGAC |
| | | GAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGG |
| | | AGGGCCUGAAGGACAAGAAGUCCCCCCUGAACCUGUCCUCCGAGCUGCAGGACGAGAUCGGCACCGCCUUCUCCCUGUUCAAG |
| | | ACCGACGAGGACAUCACCGGCCGGCUGAAGGACCGGGUGCAGCCCGAGAUCCUGGAGGCCCUGCUGAAGCACAUCUCCUUCGA |
| | | CAAGUUCGUGCAGAUCUCCCUGAAGGCCCUGCGGCGGAUCGUGCCCCUGAUGGAGCAGGGCAAGCGGUACGACGAGGCCUGCG |
| | | CCGAGAUCUACGGCGACCACUACGGCAAGAAGAACACCGAGGAGAAGAUCUACCUGCCCCCCAUCCCCGCCGACGAGAUCCGG |
| | | AACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGG |
| | | ACCGGGAGAAGGCCGCCGCCAAGUUCCGGGAGUACUUCCCCAACUUCGUGGGCGAGCCCAAGUCCAAGGACAUCCUGAAGCUG |
| | | CGGCUGUACGAGCAGCAGCACGGCAAGUGCCUGUACUCCGGCAAGGAGAUCAACCUGGUGCGGCUGAACGAGAAGGGCUACGU |
| | | GGAGAUCGACCACGCCCUGCCCUUCUCCCGGACCUGGGACGACUCCUUCAACAACAAGGUGCUGGUGCUGGGCUCCGAGAACC |
| | | AGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGUGCAACCU |
| | | GAACGACACCCGGUACGUGAACCGGUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAU |
| | | GAACGCCUUCGACGGCAAGACCAUCGACAAGGAGACCGGCAAGGUGCUGCACCAGAAGACCCACUUCCCCCAGCCCUGGGAGU |
| | | UCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCG |
| | | UGAAGCGGGUGUGGCUGACCGAGAUCAAGCUGGCCGACCUGGAGAACAUGGUGAACUACAAGAACGGCCGGGAGAUCGAGCUG |
| | | UACGAGGCCCUGAAGGCCCGGCUGGAGGCCUACGGCGGCAACGCCAAGCAGGCCUUCGACCCCAAGGACAACCCCUUCUACAA |
| | | GAAGGGCGGCCAGCUGGUGAAGGCCGUGCGGGUGGAGAAGACCCAGGAGUCCGGCGUGCUGCUGAACAAGAAGAACGCCUACA |
| | | CCAUCGCCGACAACGGCGACAUGGUGCGGGUGGACGUGUUCUGCAAGGUGGACAAGAAGGGCAAGAACCAGUACUUCAUCGUG |
| | | CCCAUCUACGCCUGGCAGGUGGCCGAGAACAUCCUGCCCGACAUCGACUGCAAGGGCUACCGGAUCGACGACUCCUACACCUU |
| | | CUGCUUCUCCCUGCACAAGUACGACCUGAUCGCCUUCCAGAAGGACGAGAAGUCCAAGGUGGAGUUCGCCUACUACAUCAACU |
| | | GCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCG |
| | | GUCCGAGUCCGCCACCCCCGAGUCCGUGUCCGGCUGGCGGCUGUUCAAGAAGAUCUCCUAGCUAGCACCAGCCUCAAGAACAC |
| | | CCGAAUGGAGUCUCUAAGCUACAUAAUACCAACUUACACUUUACAAAAUGUUGUCCCCCAAAAUGUAGCCAUUCGUAUCUGCU |
| | | CCUAAUAAAAAGAAAGUUUCUUCACAUUCUAAAAAAAAAAAAUGGAAAAAAAAAAAACGGAAAAAAAAAAAAGGUAAAAAAAA |
| | | AAAAUAUAAAAAAAAAAAACAUAAAAAAAAAAAACGAAAAAAAAAAAACGUAAAAAAAAAAAACUCAAAAAAAAAAAAGAUAA |
| | | AAAAAAAAAACCUAAAAAAAAAAAAUGUAAAAAAAAAAAAGGGAAAAAAAAAAAACGCAAAAAAAAAAAACACAAAAAAAAAA |
| | | AAUGCAAAAAAAAAAAAUCGAAAAAAAAAAAAUCUAAAAAAAAAAAACGAAAAAAAAAAAACCCAAAAAAAAAAAAGACAAAA |
| | | AAAAAAAAUAGAAAAAAAAAAAAGUUAAAAAAAAAAAACUGAAAAAAAAAAAAUUUAAAAAAAAAAAAUCUCGA |
| Exemplary | 307 | GGGAAGCUCAGAAUAAACGCUCAACUUUGGCCGGAUCUGCCACCAUGGACGGCUCCGGCGGCGGCUCCCCCAAGAAGAAGCGG |
| coding | | AAGGUGGAGGACAAGCGGCCCGCCGCCACCAAGAAGGCCGGCCAGGCCAAGAAGAAGAAGGGCGGCUCCGGCGGCGGCGCAGC |
| sequence | | AUUCAAGCCAAACUCAAUCAAUUACAUCCUGGGACUGGACAUCGGCAUCGCAUCCGUCGGGUGGGCUAUGGUCGAAAUCGACG |
| encoding | | AGGAGGAGAACCCCAUCCGCCUGAUCGAUCUGGGCGUGCGCGUGUUUGAGAGGGCAGAGGUGCCUAAGACCGGCGACAGCCUG |
| Nme1Cas9 | | GCCAUGGCACGGAGACUGGCACGCUCCGUGAGGCGCCUGACCCGGAGAAGGGCCCACAGACUGCUGAGGACACGCCGGCUGCU |
| with | | GAAGAGGGAGGGCGUGCUGCAGGCCGCCAACUUCGAUGAGAAUGGCCUGAUCAAGUCCCUGCCCAAUACCCCUUGGCAGCUGA |
| HiBiT tag | | GGGCAGCCGCCCUGGACCGCAAGCUGACACCUCUGGAGUGGUCCGCCGUGCUGCUGCACCUGAUCAAGCACCGGGGCUACCUG |
| | | UCUCAGAGAAAGAACGAGGGCGAGACAGCCGAUAAGGAGCUGGGCGCCCUGCUGAAGGGAGUGGCAGGAAAUGCACACGCCCU |
| | | GCAGACCGGCGACUUUCGCACACCAGCCGAGCUGGCCCUGAACAAGUUCGAGAAGGAGAGCGGCCACAUCCGCAAUCAGCGGU |
| | | CUGACUAUAGCCACACCUUCUCCCGGAAGGAUCUGCAGGCCGAGCUGAUCCUGCUGUUUGAGAAGCAGAAGGAGUUCGGCAAC |
| | | CCACACGUGUCUGGCGGCCUGAAGGAGGGCAUCGAGACACUGCUGAUGACACAGCGGCCCGCCCUGAGCGGCGACGCAGUGCA |
| | | GAAGAUGCUGGGACACUGCACCUUUGAGCCAGCCGAGCCCAAGGCCGCCAAGAAUACCUACACAGCCGAGCGGUUCAUCUGGC |
| | | UGACAAAGCUGAACAAUCUGAGGAUCCUGGAGCAGGGAAGCGAGCGCCCACUGACCGACACAGAGAGGGCCACCCUGAUGGAU |
| | | GAGCCCUACCGCAAGUCCAAGCUGACAUAUGCACAGGCAAGGAAGCUGCUGGGCCUGGAGGACACCGCCUUCUUUAAGGGCCU |
| | | GAGAUACGGCAAGGAUAACGCCGAGGCCUCUACACUGAUGGAGAUGAAGGCCUAUCACGCCAUCAGCAGGGCCCUGGAGAAGG |
| | | AGGGCCUGAAGGACAAGAAGUCCCCACUGAAUCUGUCUCCCGAGCUGCAGGAUGAGAUCGGCACCGCCUUUAGCCUGUUCAAG |
| | | ACCGACGAGGAUAUCACAGGCAGACUGAAGGACAGGAUCCAGCCAGAGAUCCUGGAGGCCCUGCUGAAGCACAUCAGCUUUGA |
| | | UAAGUUCGUGCAGAUCAGCCUGAAGGCCCUGCGGAGGAUCGUGCCACUGAUGGAGCAGGGCAAGAGGUACGACGAGGCCUGCG |
| | | CCGAAAUCUACGGCGAUCACUAUGGCAAGAAGAACACAGAGGAGAAAAUCUACCUGCCCCCUAUCCCCGCCGAUGAGAUCAGG |
| | | AACCCUGUGGUGCUGCGCGCCCUGUCUCAGGCAAGAAAAGUGAUCAACGGAGUGGUGCGCCGGUACGGCAGCCCCGCCAGAAU |
| | | CCACAUCGAGACAGCCAGGGAAGUGGGCAAGUCCUUUAAGGACAGAAAGGAGAUCGAGAAGAGGCAGGAGGAGAACAGAAAGG |
| | | AUAGGGAGAAGGCCGCCGCCAAGUUCAGAGAGUACUUUCCUAAUUUCGUGGGCGAGCCAAAGUCCAAGGAUAUCCUGAAGCUG |
| | | AGGCUGUACGAGCAGCAGCACGGCAAGUGUCUGUAUUCUGGCAAGGAGAUCAACCUGGGCCGCCUGAAUGAGAAGGGCUAUGU |
| | | GGAGAUCGACCACGCCCUGCCUUUUUCUCGGACCUGGGACGAUAGCUUCAACAAUAAGGUGCUGGUGCUGGGCUCUGAGAACC |
| | | AGAAUAAGGGCAACCAGACACCCUACGAGUAUUUCAACGGCAAGGACAAUAGCCGCGAGUGGCAGGAGUUUAAGGCAAGGGUG |
| | | GAGACAAGCAGGUUCCCUCGGUCCAAGAAGCAGAGAAUCCUGCUGCAGAAGUUUGACGAGGAUGGCUUCAAGGAGAGGAACCU |
| | | GAAUGACACCCGCUACGUGAAUCGGUUUCUGUGCCAGUUCGUGGCCGAUAGAAUGAGGCUGACCGGCAAGGGCAAGAAGAGAG |
| | | UGUUUGCCUCCAACGGCCAGAUCACAAAUCUGCUGAGGGGCUUCUGGGGCCUGAGAAAGGUGAGGGCAGAGAACGACAGGCAC |
| | | CACGCACUGGAUGCAGUGGUGGUGGCAUGUUCUACCGUGGCCAUGCAGCAGAAGAUCACACGCUUUGUGCGGUAUAAGGAGAU |
| | | GAAUGCCUUCGACGGCAAGACCAUCGAUAAGGAGACAGGCGAGGUGCUGCACCAGAAGACACACUUUCCUCAGCCAUGGGAGU |
| | | UCUUUGCCCAGGAAGUGAUGAUCCGGGUGUUUGGCAAGCCUGACGGCAAGCCAGAGUUCGAGGAGGCCGAUACCCUGGAGAAG |
| | | CUGAGAACACUGCUGGCAGAGAAGCUGAGCUCCAGGCCCGAGGCAGUGCACGAGUACGUGACCCCACUGUUCGUGUCUAGAGC |
| | | CCCCAACAGGAAGAUGAGCGGCCAGGGCCACAUGGAGACAGUGAAGUCCGCCAAGAGACUGGACGAGGGCGUGUCUGUGCUGA |
| | | GGGUGCCUCUGACACAGCUGAAGCUGAAGGAUCUGGAGAAGAUGGUGAAUCGCGAGCGGGAGCCAAAGCUGUAUGAGGCCCUG |
| | | AAGGCAAGGCUGGAGGCACACAAGGACGAUCCUGCCAAGGCCUUUGCCGAGCCAUUCUACAAGUAUGAUAAGGCCGGCAACAG |
| | | AACCCAGCAGGUGAAGGCCGUGAGGGUGGAGCAGGUGCAGAAGACAGGCGUGUGGGUGCGCAACCACAAUGGCAUCGCCGACA |
| | | AUGCUACCAUGGUGCGGGUGGACGUGUUUGAGAAGGGCGAUAAGUACUAUCUGGUGCCCAUCUACAGCUGGCAGGUGGCCAAG |
| | | GGCAUCCUGCCUGAUAGAGCCGUGGUGCAGGGCAAGGACGAGGAGGAUUGGCAGCUGAUCGACGAUUCCUUCAACUUUAAGUU |
| | | CUCUCUGCACCCCAAUGACCUGGUGGAAGUGAUCACCAAGAAGGCCAGGAUGUUUGGCUACUUCGCCUCCUGCCACCGCGGCA |
| | | CAGGCAACAUCAAUAUCCGGAUCCACGACCUGGAUCACAAGAUCGGCAAGAACGGCAUCCUGGAGGGCAUCGGCGUGAAGACA |
| | | GCCCUGAGCUUCCAGAAGUAUCAGAUCGACGAGCUGGGCAAGGAGAUCAGACCUUGUAGGCUGAAGAAGCGCCCACCCGUGCG |
| | | GUCCGAGUCCGCCACCCCCGAGUCCGUGUCCGGCUGGCGGCUGUUCAAGAAGAUCUCCUAGCUAGCACCAGCCUCAAGAACAC |
| | | CCGAAUGGAGUCUCUAAGCUACAUAAUACCAACUUACACUUUACAAAAUGUUGUCCCCCAAAAUGUAGCCAUUCGUAUCUGCU |
| | | CCUAAUAAAAAGAAAGUUUCUUCACAUUCUAAAAAAAAAAAAUGGAAAAAAAAAAAACGGAAAAAAAAAAAAGGUAAAAAAAA |
| | | AAAAUAUAAAAAAAAAAAACAUAAAAAAAAAAAACGAAAAAAAAAAAACGUAAAAAAAAAAAACUCAAAAAAAAAAAAGAUAA |
| | | AAAAAAAAAACCUAAAAAAAAAAAAUGUAAAAAAAAAAAAGGGAAAAAAAAAAAACGCAAAAAAAAAAAACACAAAAAAAAAA |
| | | AAUGCAAAAAAAAAAAAUCGAAAAAAAAAAAAUCUAAAAAAAAAAAACGAAAAAAAAAAAACCCAAAAAAAAAAAAGACAAAA |
| | | AAAAAAAAUAGAAAAAAAAAAAAGUUAAAAAAAAAAAACUGAAAAAAAAAAAAUUUAAAAAAAAAAAAUCUCGA |
| Exemplary | 308 | GGGAAGCUCAGAAUAAACGCUCAACUUUGGCCGGAUCUGCCACCAUGGACGGCUCCGGCGGCGGCUCCCCCAAGAAGAAGCGG |
| coding | | AAGGUGGAGGACAAGCGGCCCGCCGCCACCAAGAAGGCCGGCCAGGCCAAGAAGAAGAAGGGCGGCUCCGGCGGCGGCGCCGC |
| sequence | | CUUCAAGCCCAACUCCAUCAACUACAUCCUGGGCCUGGACAUCGGCAUCGCCUCCGUGGGCUGGGCCAUGGUGGAGAUCGACG |
| encoding | | AGGAGGAGAACCCCAUCCGGCUGAUCGACCUGGGCGUGCGGGUGUUCGAGCGGGCCGAGGUGCCCAAGACCGGCGACUCCCUG |
| Nme1Cas9 | | GCCAUGGCCCGGCGGCUGGCCCGGUCCGUGCGGCGGCUGACCCGGCGGCGGGCCCACCGGCUGCUGCGGACCCGGCGGCUGCU |
| with | | GAAGCGGGAGGGCGUGCUGCAGGCCGCCAACUUCGACGAGAACGGCCUGAUCAAGUCCCUGCCCAACACCCCCUGGCAGCUGC |
| HiBiT tag | | GGGCCGCCGCCCUGGACCGGAAGCUGACCCCCCUGGAGUGGUCCGCCGUGCUGCUGCACCUGAUCAAGCACCGGGGCUACCUG |
| | | UCCCAGCGGAAGAACGAGGGCGAGACCGCCGACAAGGAGCUGGGCGCCCUGCUGAAGGGCGUGGCCGGCAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGU |
| | | CCGACUACUCCCACACCUUCUCCCGGAAGGACCUGCAGGCCGAGCUGAUCCUGCUGUUCGAGAAGCAGAAGGAGUUCGGCAAC |
| | | CCCCACGUGUCCGGCGGCCUGAAGGAGGGCAUCGAGACCCUGCUGAUGACCCAGCGGCCCGCCCUGUCCGGCGACGCCGUGCA |
| | | GAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGAC |
| | | GAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGG |
| | | AGGGCCUGAAGGACAAGAAGUCCCCCCUGAACCUGUCCCCCGAGCUGCAGGACGAGAUCGGCACCGCCUUCUCCCUGUUCAAG |
| | | ACCGACGAGGACAUCACCGGCCGGCUGAAGGACCGGAUCCAGCCCGAGAUCCUGGAGGCCCUGCUGAAGCACAUCUCCUUCGA |
| | | CAAGUUCGUGCAGAUCUCCCUGAAGGCCCUGCGGCGGAUCGUGCCCCUGAUGGAGCAGGGCAAGCGGUACGACGAGGCCUGCG |
| | | CCGAGAUCUACGGCGACCACUACGGCAAGAAGAACACCGAGGAGAAGAUCUACCUGCCCCCCAUCCCCGCCGACGAGAUCCGG |
| | | AACCCCGUGGUGCUGCGGGCCCUGUCCCAGGCCCGGAAGGUGAUCAACGGCGUGGUGCGGCGGUACGGCUCCCCCGCCCGGAU |
| | | CCACAUCGAGACCGCCCGGGAGGUGGGCAAGUCCUUCAAGGACCGGAAGGAGAUCGAGAAGCGGCAGGAGGAGAACCGGAAGG |
| | | ACCGGGAGAAGGCCGCCGCCAAGUUCCGGGAGUACUUCCCCAACUUCGUGGGCGAGCCCAAGUCCAAGGACAUCCUGAAGCUG |
| | | CGGCUGUACGAGCAGCAGCACGGCAAGUGCCUGUACUCCGGCAAGGAGAUCAACCUGGGCCGGCUGAACGAGAAGGGCUACGU |
| | | GGAGAUCGACCACGCCCUGCCCUUCUCCCGGACCUGGGACGACUCCUUCAACAACAAGGUGCUGGUGCUGGGCUCCGAGAACC |
| | | AGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGCGGAACCU |
| | | GAACGACACCCGGUACGUGAACCGGUUCCUGUGCCAGUUCGUGGCCGACCGGAUGCGGCUGACCGGCAAGGGCAAGAAGCGGG |
| | | UGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAU |
| | | GAACGCCUUCGACGGCAAGACCAUCGACAAGGAGACCGGCGAGGUGCUGCACCAGAAGACCCACUUCCCCCAGCCCUGGGAGU |
| | | UCUUCGCCCAGGAGGUGAUGAUCCGGGUGUUCGGCAAGCCCGACGGCAAGCCCGAGUUCGAGGAGGCCGACACCCUGGAGAAG |
| | | CUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCCAGGGCCACAUGGAGACCGUGAAGUCCGCCAAGCGGCUGGACGAGGGCGUGUCCGUGCUGC |
| | | GGGUGCCCCUGACCCAGCUGAAGCUGAAGGACCUGGAGAAGAUGGUGAACCGGGAGCGGGAGCCCAAGCUGUACGAGGCCCUG |
| | | AAGGCCCGGCUGGAGGCCCACAAGGACGACCCCGCCAAGGCCUUCGCCGAGCCCUUCUACAAGUACGACAAGGCCGGCAACCG |
| | | GACCCAGCAGGUGAAGGCCGUGCGGGUGGAGCAGGUGCAGAAGACCGGCGUGUGGGUGCGGAACCACAACGGCAUCGCCGACA |
| | | ACGCCACCAUGGUGCGGGUGGACGUGUUCGAGAAGGGCGACAAGUACUACCUGGUGCCCAUCUACUCCUGGCAGGUGGCCAAG |
| | | GGCAUCCUGCCCGACCGGGCCGUGGUGCAGGGCAAGGACGAGGAGGACUGGCAGCUGAUCGACGACUCCUUCAACUUCAAGUU |
| | | CUCCCUGCACCCCAACGACCUGGUGGAGGUGAUCACCAAGAAGGCCCGGAUGUUCGGCUACUUCGCCUCCUGCCACCGGGGCA |
| | | CCGGCAACAUCAACAUCCGGAUCCACGACCUGGACCACAAGAUCGGCAAGAACGGCAUCCUGGAGGGCAUCGGCGUGAAGACC |
| | | GCCCUGUCCUUCCAGAAGUACCAGAUCGACGAGCUGGGCAAGGAGAUCCGGCCCUGCCGGCUGAAGAAGCGGCCCCCCGUGCG |
| | | GUCCGAGUCCGCCACCCCCGAGUCCGUGUCCGGCUGGCGGCUGUUCAAGAAGAUCUCCUAGCUAGCACCAGCCUCAAGAACAC |
| | | CCGAAUGGAGUCUCUAAGCUACAUAAUACCAACUUACACUUUACAAAAUGUUGUCCCCCAAAAUGUAGCCAUUCGUAUCUGCU |
| | | CCUAAUAAAAAGAAAGUUUCUUCACAUUCUAAAAAAAAAAAAUGGAAAAAAAAAAAACGGAAAAAAAAAAAAGGUAAAAAAAA |
| | | AAAAUAUAAAAAAAAAAAACAUAAAAAAAAAAAACGAAAAAAAAAAAACGUAAAAAAAAAAAACUCAAAAAAAAAAAAGAUAA |
| | | AAAAAAAAAACCUAAAAAAAAAAAAUGUAAAAAAAAAAAAGGGAAAAAAAAAAAACGCAAAAAAAAAAAACACAAAAAAAAAA |
| | | AAUGCAAAAAAAAAAAAUCGAAAAAAAAAAAAUCUAAAAAAAAAAAACGAAAAAAAAAAAACCCAAAAAAAAAAAAGACAAAA |
| | | AAAAAAAAUAGAAAAAAAAAAAAGUUAAAAAAAAAAAACUGAAAAAAAAAAAAUUUAAAAAAAAAAAAUCUCGA |
| Exemplary | 309 | GGGAAGCUCAGAAUAAACGCUCAACUUUGGCCGGAUCUGCCACCAUGGACGGCUCCGGCGGCGGCUCCCCCAAGAAGAAGCGG |
| coding | | AAGGUGGAGGACAAGCGGCCCGCCGCCACCAAGAAGGCCGGCCAGGCCAAGAAGAAGAAGGGCGGCUCCGGCGGCGCCGCCUU |
| sequence | | CAAGCCCAACCCCAUCAACUACAUCCUGGGCCUGGACAUCGGCAUCGCCUCCGUGGGCUGGGCCAUGGUGGAGAUCGACGAGG |
| encoding | | AGGAGAACCCCAUCCGGCUGAUCGACCUGGGCGUGCGGGUGUUCGAGCGGGCCGAGGUGCCCAAGACCGGCGACUCCCUGGCC |
| Nme3Cas9 | | AUGGCCCGGCGGCUGGCCCGGUCCGUGCGGCGGCUGACCCGGCGGCGGGCCCACCGGCUGCUGCGGGCCCGGCGGCUGCUGAA |
| with | | GCGGGAGGGCGUGCUGCAGGCCGCCGACUUCGACGAGAACGGCCUGAUCAAGUCCCUGCCCAACACCCCCUGGCAGCUGCGGG |
| HiBiT tag | | CCGCCGCCCUGGACCGGAAGCUGACCCCCCUGGAGUGGUCCGCCGUGCUGCUGCACCUGAUCAAGCACCGGGGCUACCUGUCC |
| | | CAGCGGAAGAACGAGGGCGAGACCGCCGACAAGGAGCUGGGCGCCCUGCUGAAGGGCGUGGCCGACAACGCCCACGCCCUGCA |
| | | GACCGGCGACUUCCGGACCCCCGCCGAGCUGGCCCUGAACAAGUUCGAGAAGGAGUGCGGCCACAUCCGGAACCAGCGGGGCG |
| | | ACUACUCCCACACCUUCUCCCGGAAGGACCUGCAGGCCGAGCUGAACCUGCUGUUCGAGAAGCAGAAGGAGUUCGGCAACCCC |
| | | CACGUGUCCGGCGGCCUGAAGGAGGGCAUCGAGACCCUGCUGAUGACCCAGCGGCCCGCCCUGUCCGGCGACGCCGUGCAGAA |
| | | GAUGCUGGGCCACUGCACCUUCGAGCCCGCCGAGCCCAAGGCCGCCAAGAACACCUACACCGCCGAGCGGUUCAUCUGGCUGA |
| | | CCAAGCUGAACAACCUGCGGAUCCUGGAGCAGGGCUCCGAGCGGCCCCUGACCGACACCGAGCGGGCCACCCUGAUGGACGAG |
| | | CCCUACCGGAAGUCCAAGCUGACCUACGCCCAGGCCCGGAAGCUGCUGUCCCUGGAGGACACCGCCUUCUUCAAGGGCCUGCG |
| | | GUACGGCAAGGACAACGCCGAGGCCUCCACCCUGAUGGAGAUGAAGGCCUACCACACCAUCUCCCGGGCCCUGGAGAAGGAGG |
| | | GCCUGAAGGACAAGAAGUCCCCCCUGAACCUGUCCCCCGAGCUGCAGGACGAGAUCGGCACCGCCUUCUCCCUGUUCAAGACC |
| | | GACGAGGACAUCACCGGCCGGCUGAAGGACCGGAUCCAGCCCGAGAUCCUGGAGGCCCUGCUGAAGCACAUCUCCUUCGACAA |
| | | GUUCGUGCAGAUCUCCCUGAAGGCCCUGCGGCGGAUCGUGCCCCUGAUGGAGCAGGGCAAGCGGUACGACGAGGCCUGCGCCG |
| | | AGAUCUACGGCGACCACUACGGCAAGAAGAACACCGAGGAGAAGAUCUACCUGCCCCCCAUCCCCGCCGACGAGAUCCGGAAC |
| | | CCCGUGGUGCUGCGGGCCCUGUCCCAGGCCCGGAAGGUGAUCAACGGCGUGGUGCGGCGGUACGGCUCCCCCGCCCGGAUCCA |
| | | CAUCGAGACCGCCCGGGAGGUGGGCAAGUCCUUCAAGGACCGGAAGGAGAUCGAGAAGCGGCAGGAGGAGAACCGGAAGGACC |
| | | GGGAGAAGGCCGCCGCCAAGUUCCGGGAGUACUUCCCCAACUUCGUGGGCGAGCCCAAGUCCAAGGACAUCCUGAAGCUGCGG |
| | | CUGUACGAGCAGCAGCACGGCAAGUGCCUGUACUCCGGCAAGGAGAUCAACCUGGGCCGGCUGAACGAGAAGGGCUACGUGGA |
| | | GAUCGACCACGCCCUGCCCUUCUCCCGGACCUGGGACGACUCCUUCAACAACAAGGUGCUGGUGCUGGGCUCCGAGAACCAGA |
| | | ACAAGGGCAACCAGACCCCCUACGAGUACUUCAACGGCAAGGACAACUCCCGGGAGUGGCAGGAGUUCAAGGCCCGGGUGGAG |
| | | ACCUCCCGGUUCCCCCGGUCCAAGAAGCAGCGGAUCCUGCUGCAGAAGUUCGACGAGGACGGCUUCAAGGAGCGGAACCUGAA |
| | | CGACACCCGGUACGUGAACCGGUUCCUGUGCCAGUUCGUGGCCGACCGGAUGCGGCUGACCGGCAAGGGCAAGAAGCGGGUGU |
| | | UCGCCUCCAACGGCCAGAUCACCAACCUGCUGCGGGGCUUCUGGGGCCUGCGGAAGGUGCGGGCCGAGAACGACCGGCACCAC |
| | | GCCCUGGACGCCGUGGUGGUGGCCUGCUCCACCGUGGCCAUGCAGCAGAAGAUCACCCGGUUCGUGCGGUACAAGGAGAUGAA |
| | | CGCCUUCGACGGCAAGACCAUCGACAAGGAGACCGGCGAGGUGCUGCACCAGAAGACCCACUUCCCCCAGCCCUGGGAGUUCU |
| | | UCGCCCAGGAGGUGAUGAUCCGGGUGUUCGGCAAGCCCGACGGCAAGCCCGAGUUCGAGGAGGCCGACACCCCCGAGAAGCUG |
| | | CGGACCCUGCUGGCCGAGAAGCUGUCCUCCCGGCCCGAGGCCGUGCACGAGUACGUGACCCCCCUGUUCGUGUCCCGGGCCCC |
| | | CAACCGGAAGAUGUCCGGCCAGGGCCACAUGGAGACCGUGAAGUCCGCCAAGCGGCUGGACGAGGGCGUGUCCGUGCUGCGGG |
| | | UGCCCCUGACCCAGCUGAAGCUGAAGGACCUGGAGAAGAUGGUGAACCGGGAGCGGGAGCCCAAGCUGUACGAGGCCCUGAAG |
| | | GCCCGGCUGGAGGCCCACAAGGACGACCCCGCCAAGGCCUUCGCCGAGCCCUUCUACAAGUACGACAAGGCCGGCAACCGGAC |
| | | CCAGCAGGUGAAGGCCGUGCGGGUGGAGCAGGUGCAGAAGACCGGCGUGUGGGUGCGGAACCACAACGGCAUCGCCGACAACG |
| | | CCACCAUGGUGCGGGUGGACGUGUUCGAGAAGGGCGACAAGUACUACCUGGUGCCCAUCUACUCCUGGCAGGUGGCCAAGGGC |
| | | AUCCUGCCCGACCGGGCCGUGGUGGCCUACGCCGACGAGGAGGACUGGACCGUGAUCGACGAGUCCUUCCGGUUCAAGUUCGU |
| | | GCUGUACUCCAACGACCUGAUCAAGGUGCAGCUGAAGAAGGACUCCUUCCUGGGCUACUUCUCCGGCCUGGACCGGGCCACCG |
| | | GCGCCAUCUCCCUGCGGGAGCACGACCUGGAGAAGUCCAAGGGCAAGGACGGCAUGCACCGGAUCGGCGUGAAGACCGCCCUG |
| | | UCCUUCCAGAAGUACCAGAUCGACGAGAUGGGCAAGGAGAUCCGGCCCUGCCGGCUGAAGAAGCGGCCCCCCGUGCGGUCCGA |
| | | GUCCGCCACCCCCGAGUCCGUGUCCGGCUGGCGGCUGUUCAAGAAGAUCUCCUAGCUAGCACCAGCCUCAAGAACACCCGAAU |
| | | GGAGUCUCUAAGCUACAUAAUACCAACUUACACUUUACAAAAUGUUGUCCCCCAAAAUGUAGCCAUUCGUAUCUGCUCCUAAU |
| | | AAAAAGAAAGUUUCUUCACAUUCUCUCGAGAAAAAAAAAAAAUGGAAAAAAAAAAAACGGAAAAAAAAAAAAGGUAAAAAAAA |
| | | AAAAUAUAAAAAAAAAAAACAUAAAAAAAAAAAACGAAAAAAAAAAAACGUAAAAAAAAAAAACUCAAAAAAAAAAAAGAUAA |
| | | AAAAAAAAAACCUAAAAAAAAAAAAUGUAAAAAAAAAAAAGGGAAAAAAAAAAAACGCAAAAAAAAAAAACACAAAAAAAAAA |
| | | AAUGCAAAAAAAAAAAAUCGAAAAAAAAAAAAUCUAAAAAAAAAAAACGAAAAAAAAAAAACCCAAAAAAAAAAAAGACAAAA |
| | | AAAAAAAAUAGAAAAAAAAAAAAGUUAAAAAAAAAAAACUGAAAAAAAAAAAAUUUAAAAAAAAAAAAUCUAG |
| Exemplary | 310 | MDGSGGGSPKKKRKVEDKRPAATKKAGQAKKKKGGSGGGAAFKPNPINYILGLDIGIASVGWAMVEIDEEENPIRLIDLGVRV |
| amino | | FERAEVPKTGDSLAMARRLARSVRRLTRRRAHRLLRARRLLKREGVLQAADEDENGLIKSLPNTPWQLRAAALDRKLTPLEWS |
| acid | | AVLLHLIKHRGYLSQRKNEGETADKELGALLKGVANNAHALQTGDERTPAELALNKFEKESGHIRNQRGDYSHTFSRKDLQAE |
| sequence | | LILLFEKQKEFGNPHVSGGLKEGIETLLMTQRPALSGDAVQKMLGHCTFEPAEPKAAKNTYTAERFIWLTKLNNLRILEQGSE |
| for | | RPLTDTERATLMDEPYRKSKLTYAQARKLLGLEDTAFFKGLRYGKDNAEASTLMEMKAYHAISRALEKEGLKDKKSPLNLSSE |
| Nme2Cas9 | | LQDEIGTAFSLFKTDEDITGRLKDRVQPEILEALLKHISFDKFVQISLKALRRIVPLMEQGKRYDEACAEIYGDHYGKKNTEE |
| | | KIYLPPIPADEIRNPVVLRALSQARKVINGVVRRYGSPARIHIETAREVGKSFKDRKEIEKRQEENRKDREKAAAKFREYFPN |
| | | FVGEPKSKDILKLRLYEQQHGKCLYSGKEINLVRLNEKGYVEIDHALPFSRTWDDSFNNKVLVLGSENQNKGNQTPYEYENGK |
| | | DNSREWQEFKARVETSRFPRSKKQRILLQKFDEDGEKECNLNDTRYVNRFLCQFVADHILLTGKGKRRVFASNGQITNLLRGF |
| | | WGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLE |
| | | NMVNYKNGREIELYEALKARLEAYGGNAKQAFDPKDNPFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKYDLIAFQKDEKSKVEFAYYINCDSSNGREYLAWHDK |
| | | GSKEQQFRISTQNLVLIQKYQVNELGKEIRPCRLKKRPPVR* |
| Exemplary | 311 | MAAFKPNSINYILGLDIGIASVGWAMVEIDEEENPIRLIDLGVRVFERAEVPKTGDSLAMARRLARSVRRLTRRRAHRLLRTR |
| amino | | RLLKREGVLQAANFDENGLIKSLPNTPWQLRAAALDRKLTPLEWSAVLLHLIKHRGYLSQRKNEGETADKELGALLKGVAGNA |
| acid | | HALQTGDFRTPAELALNKFEKESGHIRNQRSDYSHTFSRKDLQAELILLFEKQKEFGNPHVSGGLKEGIETLLMTQRPALSGD |
| sequence | | AVQKMLGHCTFEPAEPKAAKNTYTAERFIWLTKLNNLRILEQGSERPLTDTERATLMDEPYRKSKLTYAQARKLLGLEDTAFF |
| for | | KGLRYGKDNAEASTLMEMKAYHAISRALEKEGLKDKKSPLNLSPELQDEIGTAFSLFKTDEDITGRLKDRIQPEILEALLKHI |
| Nme1Cas9 | | SFDKFVQISLKALRRIVPLMEQGKRYDEACAEIYGDHYGKKNTEEKIYLPPIPADEIRNPVVLRALSQARKVINGVVRRYGSP |
| | | ARIHIETAREVGKSFKDRKEIEKRQEENRKDREKAAAKFREYFPNFVGEPKSKDILKLRLYEQQHGKCLYSGKEINLGRLNEK |
| | | GYVEIDHALPFSRTWDDSFNNKVLVLGSENQNKGNQTPYEYENGKDNSREWQEFKARVETSRFPRSKKQRILLQKFDEDGEKE |
| | | RNLNDTRYVNRFLCQFVADRMRLTGKGKKRVFASNGQITNLLRGFWGLRKVRAENDRHHALDAVVVACSTVAMQQKITRFVRY |
| | | KEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYDKA |
| | | GNRTQQVKAVRVEQVQKTGVWVRNHNGIADNATMVRVDVFEKGDKYYLVPIYSWQVAKGILPDRAVVQGKDEEDWQLIDDSFN |
| | | FKFSLHPNDLVEVITKKARMFGYFASCHRGTGNINIRIHDLDHKIGKNGILEGIGVKTALSFQKYQIDELGKEIRPCRLKKRP |
| | | PVRSGKRTADGSEFESPKKKRKVE* |
| Exemplary | 312 | MDGSGGGSPKKKRKVEDKRPAATKKAGQAKKKKGGSGGGAAFKPNPINYILGLDIGIASVGWAMVEIDEEENPIRLIDLGVRV |
| amino | | FERAEVPKTGDSLAMARRLARSVRRLTRRRAHRLLRARRLLKREGVLQAADEDENGLIKSLPNTPWQLRAAALDRKLTPLEWS |
| acid | | AVLLHLIKHRGYLSQRKNEGETADKELGALLKGVANNAHALQTGDERTPAELALNKFEKESGHIRNQRGDYSHTFSRKDLQAE |
| sequence | | LILLFEKQKEFGNPHVSGGLKEGIETLLMTQRPALSGDAVQKMLGHCTFEPAEPKAAKNTYTAERFIWLTKLNNLRILEQGSE |
| for | | RPLTDTERATLMDEPYRKSKLTYAQARKLLGLEDTAFFKGLRYGKDNAEASTLMEMKAYHAISRALEKEGLKDKKSPLNLSSE |
| Nme2Cas9 | | LQDEIGTAFSLEKTDEDITGRLKDRVQPEILEALLKHISFDKFVQISLKALRRIVPLMEQGKRYDEACAEIYGDHYGKKNTEE |
| with | | KIYLPPIPADEIRNPVVLRALSQARKVINGVVRRYGSPARIHIETAREVGKSFKDRKEIEKRQEENRKDREKAAAKFREYFPN |
| HiBiT tag | | FVGEPKSKDILKLRLYEQQHGKCLYSGKEINLVRLNEKGYVEIDHALPFSRTWDDSFNNKVLVLGSENQNKGNQTPYEYENGK |
| | | DNSREWQEFKARVETSRFPRSKKQRILLQKFDEDGFKECNLNDTRYVNRFLCQFVADHILLTGKGKRRVFASNGQITNLLRGE |
| | | WGLRKVRAENDRHHALDAVVVACSTVAMQQKITRFVRYKEMNAFDGKTIDKETGKVLHQKTHFPQPWEFFAQEVMIRVFGKPD |
| | | GKPEFEEADTPEKLRTLLAEKLSSRPEAVHEYVTPLFVSRAPNRKMSGAHKDTLRSAKRFVKHNEKISVKRVWLTEIKLADLE |
| | | NMVNYKNGREIELYEALKARLEAYGGNAKQAFDPKDNPFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKYDLIAFQKDEKSKVEFAYYINCDSSNGRFYLAWHDK |
| | | GSKEQQFRISTQNLVLIQKYQVNELGKEIRPCRLKKRPPVRSESATPESVSGWRLFKKIS* |
| Exemplary | 313 | MDGSGGGSPKKKRKVEDKRPAATKKAGQAKKKKGGSGGGAAFKPNSINYILGLDIGIASVGWAMVEIDEEENPIRLIDLGVRV |
| amino | | FERAEVPKTGDSLAMARRLARSVRRLTRRRAHRLLRTRRLLKREGVLQAANEDENGLIKSLPNTPWQLRAAALDRKLTPLEWS |
| acid | | AVLLHLIKHRGYLSQRKNEGETADKELGALLKGVAGNAHALQTGDFRTPAELALNKFEKESGHIRNQRSDYSHTESRKDLQAE |
| sequence | | LILLFEKQKEFGNPHVSGGLKEGIETLLMTQRPALSGDAVQKMLGHCTFEPAEPKAAKNTYTAERFIWLTKLNNLRILEQGSE |
| for | | RPLTDTERATLMDEPYRKSKLTYAQARKLLGLEDTAFFKGLRYGKDNAEASTLMEMKAYHAISRALEKEGLKDKKSPLNLSPE |
| Nme1Cas9 | | LQDEIGTAFSLFKTDEDITGRLKDRIQPEILEALLKHISFDKFVQISLKALRRIVPLMEQGKRYDEACAEIYGDHYGKKNTEE |
| with | | KIYLPPIPADEIRNPVVLRALSQARKVINGVVRRYGSPARIHIETAREVGKSFKDRKEIEKRQEENRKDREKAAAKFREYFPN |
| HiBiT tag | | FVGEPKSKDILKLRLYEQQHGKCLYSGKEINLGRLNEKGYVEIDHALPFSRTWDDSFNNKVLVLGSENQNKGNQTPYEYENGK |
| | | DNSREWQEFKARVETSRFPRSKKQRILLQKFDEDGEKERNLNDTRYVNRFLCQFVADRMRLTGKGKKRVFASNGQITNLLRGE |
| | | WGLRKVRAENDRHHALDAVVVACSTVAMQQKITRFVRYKEMNAFDGKTIDKETGEVLHQKTHFPQPWEFFAQEVMIRVFGKPD |
| | | GKPEFEEADTLEKLRTLLAEKLSSRPEAVHEYVTPLFVSRAPNRKMSGQGHMETVKSAKRLDEGVSVLRVPLTQLKLKDLEKM |
| | | VNREREPKLYEALKARLEAHKDDPAKAFAEPFYKYDKAGNRTQQVKAVRVEQVQKTGVWVRNHNGIADNATMVRVDVFEKGDK |
| | | YYLVPIYSWQVAKGILPDRAVVQGKDEEDWQLIDDSFNFKESLHPNDLVEVITKKARMEGYFASCHRGTGNINIRIHDLDHKI |
| | | GKNGILEGIGVKTALSFQKYQIDELGKEIRPCRLKKRPPVRSESATPESVSGWRLFKKIS* |
| Exemplary | 314 | MDGSGGGSPKKKRKVEDKRPAATKKAGQAKKKKGGSGGGAAFKPNSINYILGLDIGIASVGWAMVEIDEEENPIRLIDLGVRV |
| amino | | FERAEVPKTGDSLAMARRLARSVRRLTRRRAHRLLRTRRLLKREGVLQAANEDENGLIKSLPNTPWQLRAAALDRKLTPLEWS |
| acid | | AVLLHLIKHRGYLSQRKNEGETADKELGALLKGVAGNAHALQTGDFRTPAELALNKFEKESGHIRNQRSDYSHTFSRKDLQAE |
| sequence | | LILLFEKQKEFGNPHVSGGLKEGIETLLMTQRPALSGDAVQKMLGHCTFEPAEPKAAKNTYTAERFIWLTKLNNLRILEQGSE |
| for | | RPLTDTERATLMDEPYRKSKLTYAQARKLLGLEDTAFFKGLRYGKDNAEASTLMEMKAYHAISRALEKEGLKDKKSPLNLSPE |
| Nme1Cas 9 | | LQDEIGTAFSLFKTDEDITGRLKDRIQPEILEALLKHISFDKFVQISLKALRRIVPLMEQGKRYDEACAEIYGDHYGKKNTEE |
| with | | KIYLPPIPADEIRNPVVLRALSQARKVINGVVRRYGSPARIHIETAREVGKSFKDRKEIEKRQEENRKDREKAAAKFREYFPN |
| HiBiT tag | | FVGEPKSKDILKLRLYEQQHGKCLYSGKEINLGRLNEKGYVEIDHALPFSRTWDDSFNNKVLVLGSENQNKGNQTPYEYENGK |
| | | DNSREWQEFKARVETSRFPRSKKQRILLQKEDEDGFKERNLNDTRYVNRFLCQFVADRMRLTGKGKKRVFASNGQITNLLRGF |
| | | WGLRKVRAENDRHHALDAVVVACSTVAMQQKITRFVRYKEMNAFDGKTIDKETGEVLHQKTHFPQPWEFFAQEVMIRVFGKPD |
| | | GKPEFEEADTLEKLRTLLAEKLSSRPEAVHEYVTPLFVSRAPNRKMSGQGHMETVKSAKRLDEGVSVLRVPLTQLKLKDLEKM |
| | | VNREREPKLYEALKARLEAHKDDPAKAFAEPFYKYDKAGNRTQQVKAVRVEQVQKTGVWVRNHNGIADNATMVRVDVFEKGDK |
| | | YYLVPIYSWQVAKGILPDRAVVQGKDEEDWQLIDDSFNFKFSLHPNDLVEVITKKARMFGYFASCHRGTGNINIRIHDLDHKI |
| | | GKNGILEGIGVKTALSFQKYQIDELGKEIRPCRLKKRPPVRSESATPESVSGWRLFKKIS* |
| Exemplary | 315 | MDGSGGGSPKKKRKVEDKRPAATKKAGQAKKKKGGSGGAAFKPNPINYILGLDIGIASVGWAMVEIDEEENPIRLIDLGVRVE |
| amino | | ERAEVPKTGDSLAMARRLARSVRRLTRRRAHRLLRARRLLKREGVLQAADFDENGLIKSLPNTPWQLRAAALDRKLTPLEWSA |
| acid | | VLLHLIKHRGYLSQRKNEGETADKELGALLKGVADNAHALQTGDERTPAELALNKFEKECGHIRNQRGDYSHTFSRKDLQAEL |
| sequence | | NLLFEKQKEFGNPHVSGGLKEGIETLLMTQRPALSGDAVQKMLGHCTFEPAEPKAAKNTYTAERFIWLTKLNNLRILEQGSER |
| for | | PLTDTERATLMDEPYRKSKLTYAQARKLLSLEDTAFFKGLRYGKDNAEASTLMEMKAYHTISRALEKEGLKDKKSPLNLSPEL |
| Nme3Cas9 | | QDEIGTAFSLEKTDEDITGRLKDRIQPEILEALLKHISFDKFVQISLKALRRIVPLMEQGKRYDEACAEIYGDHYGKKNTEEK |
| with | | IYLPPIPADEIRNPVVLRALSQARKVINGVVRRYGSPARIHIETAREVGKSFKDRKEIEKRQEENRKDREKAAAKFREYFPNF |
| HiBiT tag | | VGEPKSKDILKLRLYEQQHGKCLYSGKEINLGRLNEKGYVEIDHALPFSRTWDDSFNNKVLVLGSENQNKGNQTPYEYENGKD |
| | | NSREWQEFKARVETSRFPRSKKQRILLQKFDEDGFKERNLNDTRYVNRFLCQFVADRMRLTGKGKKRVFASNGQITNLLRGFW |
| | | GLRKVRAENDRHHALDAVVVACSTVAMQQKITRFVRYKEMNAFDGKTIDKETGEVLHQKTHFPQPWEFFAQEVMIRVFGKPDG |
| | | KPEFEEADTPEKLRTLLAEKLSSRPEAVHEYVTPLFVSRAPNRKMSGQGHMETVKSAKRLDEGVSVLRVPLTQLKLKDLEKMV |
| | | NREREPKLYEALKARLEAHKDDPAKAFAEPFYKYDKAGNRTQQVKAVRVEQVQKTGVWVRNHNGIADNATMVRVDVFEKGDKY |
| | | YLVPIYSWQVAKGILPDRAVVAYADEEDWTVIDESFRFKFVLYSNDLIKVQLKKDSFLGYESGLDRATGAISLREHDLEKSKG |
| | | KDGMHRIGVKTALSFQKYQIDEMGKEIRPCRLKKRPPVRSESATPESVSGWRLFKKIS* |
| Exemplary | 316 | augGACGGCUCCGGCGGCGGCUCCCCCAAGAAGAAGCGGAAGGUGGAGGACAAGCGGCCCGCCGCCACCAAGAAGGCCGGCCA |
| open | | GGCCAAGAAGAAGAAGGGCGGCUCCGGCGGCGGCgccgccuucaagcccaaccccaucaacuacauccugggccuggacaucg |
| reading | | gcaucgccuccgugggcugggccaugguggagaucgacgaggaggagaaccccauccggcugaucgaccugggcgugcgggug |
| frame for | | uucgagcgggccgaggugcccaagaccggcgacucccuggccauggcccggcggcuggcccgguccgugcggcggcugacccg |
| Nme2Cas9 | | gcggcgggcccaccggcugcugcgggcccggcggcugcugaagcgggagggcgugcugcaggccgccgacuucgacgagaacg |
| | | gccugaucaagucccugcccaacacccccuggcagcugcgggccgccgcccuggaccggaagcugaccccccuggaguggucc |
| | | gccgugcugcugcaccugaucaagcaccggggcuaccugucccagcggaagaacgagggcgagaccgccgacaaggagcuggg |
| | | cgcccugcugaagggcguggccaacaacgcccacgcccugcagaccggcgacuuccggacccccgccgagcuggcccugaaca |
| | | aguucgagaaggaguccggccacauccggaaccagcggggcgacuacucccacaccuucucccggaaggaccugcaggccgag |
| | | cugauccugcuguucgagaagcagaaggaguucggcaacccccacguguccggcggccugaaggagggcaucgagacccugcu |
| | | gaugacccagcggcccgcccuguccggcgacgccgugcagaagaugcugggccacugcaccuucgagcccgccgagcccaagg |
| | | ccgccaagaacaccuacaccgccgagcgguucaucuggcugaccaagcugaacaaccugcggauccuggagcagggcuccgag |
| | | cggccccugaccgacaccgagcgggccacccugauggacgagcccuaccggaaguccaagcugaccuacgcccaggcccggaa |
| | | gcugcugggccuggaggacaccgccuucuucaagggccugcgguacggcaaggacaacgccgaggccuccacccugauggaga |
| | | ugaaggccuaccacgccaucucccgggcccuggagaaggagggccugaaggacaagaaguccccccugaaccuguccuccgag |
| | | cugcaggacgagaucggcaccgccuucucccuguucaagaccgacgaggacaucaccggccggcugaaggaccgggugcagcc |
| | | cgagauccuggaggcccugcugaagcacaucuccuucgacaaguucgugcagaucucccugaaggcccugcggcggaucgugc |
| | | cccugauggagcagggcaagcgguacgacgaggccugcgccgagaucuacggcgaccacuacggcaagaagaacaccgaggag |
| | | aagaucuaccugccccccauccccgccgacgagauccggaaccccguggugcugcgggcccugucccaggcccggaaggugau |
| | | caacggcguggugcggcgguacggcucccccgcccggauccacaucgagaccgcccgggaggugggcaaguccuucaaggacc |
| | | ggaaggagaucgagaagcggcaggaggagaaccggaaggaccgggagaaggccgccgccaaguuccgggaguacuuccccaac |
| | | uucgugggcgagcccaaguccaaggacauccugaagcugcggcuguacgagcagcagcacggcaagugccuguacuccggcaa |
| | | ggagaucaaccuggugcggcugaacgagaagggcuacguggagaucgaccacgcccugcccuucucccggaccugggacgacu |
| | | ccuucaacaacaaggugcuggugcugggcuccgagaaccagaacaagggcaaccagacccccuacgaguacuucaacggcaag |
| | | gacaacucccgggaguggcaggaguucaaggcccggguggagaccucccgguucccccgguccaagaagcagcggauccugcu |
| | | gcagaaguucgacgaggacggcuucaaggagugcaaccugaacgacacccgguacgugaaccgguuccugugccaguucgugg |
| | | ccgaccacauccugcugaccggcaagggcaagcggcggguguucgccuccaacggccagaucaccaaccugcugcggggcuuc |
| | | uggggccugcggaaggugcgggccgagaacgaccggcaccacgcccuggacgccguggugguggccugcuccaccguggccau |
| | | gcagcagaagaucacccgguucgugcgguacaaggagaugaacgccuucgacggcaagaccaucgacaaggagaccggcaagg |
| | | ugcugcaccagaagacccacuucccccagcccugggaguucuucgcccaggaggugaugauccggguguucggcaagcccgac |
| | | ggcaagcccgaguucgaggaggccgacacccccgagaagcugcggacccugcuggccgagaagcuguccucccggcccgaggc |
| | | cgugcacgaguacgugaccccccuguucgugucccgggcccccaaccggaagauguccggcgcccacaaggacacccugcggu |
| | | ccgccaagcgguucgugaagcacaacgagaagaucuccgugaagcggguguggcugaccgagaucaagcuggccgaccuggag |
| | | aacauggugaacuacaagaacggccgggagaucgagcuguacgaggcccugaaggcccggcuggaggccuacggcggcaacgc |
| | | caagcaggccuucgaccccaaggacaaccccuucuacaagaagggcggccagcuggugaaggccgugcggguggagaagaccc |
| | | aggaguccggcgugcugcugaacaagaagaacgccuacaccaucgccgacaacggcgacauggugcggguggacguguucugc |
| | | aagguggacaagaagggcaagaaccaguacuucaucgugcccaucuacgccuggcagguggccgagaacauccugcccgacau |
| | | cgacugcaagggcuaccggaucgacgacuccuacaccuucugcuucucccugcacaaguacgaccugaucgccuuccagaagg |
| | | acgagaaguccaagguggaguucgccuacuacaucaacugcgacuccuccaacggccgguucuaccuggccuggcacgacaag |
| | | ggcuccaaggagcagcaguuccggaucuccacccagaaccuggugcugauccagaaguaccaggugaacgagcugggcaagga |
| | | gauccggcccugccggcugaagaagcggccccccgugcgguag |
| Exemplary | 317 | AUGGCAGCAUUCAAGCCGAACUCGAUCAACUACAUCCUGGGACUGGACAUCGGAAUCGCAUCGGUCGGAUGGGCAAUGGUCGA |
| open | | AAUCGACGAAGAAGAAAACCCGAUCAGACUGAUCGACCUGGGAGUCAGAGUCUUCGAAAGAGCAGAAGUCCCGAAGACAGGAG |
| reading | | ACUCGCUGGCAAUGGCAAGAAGACUGGCAAGAUCGGUCAGAAGACUGACAAGAAGAAGAGCACACAGACUGCUGAGAACAAGA |
| frame for | | AGACUGCUGAAGAGAGAAGGAGUCCUGCAGGCAGCAAACUUCGACGAAAACGGACUGAUCAAGUCGCUGCCGAACACACCGUG |
| Nme1Cas9 | | GCAGCUGAGAGCAGCAGCACUGGACAGAAAGCUGACACCGCUGGAAUGGUCGGCAGUCCUGCUGCACCUGAUCAAGCACAGAG |
| | | GAUACCUGUCGCAGAGAAAGAACGAAGGAGAAACAGCAGACAAGGAACUGGGAGCACUGCUGAAGGGAGUCGCAGGAAACGCA |
| | | CACGCACUGCAGACAGGAGACUUCAGAACACCGGCAGAACUGGCACUGAACAAGUUCGAAAAGGAAUCGGGACACAUCAGAAA |
| | | CCAGAGAUCGGACUACUCGCACACAUUCUCGAGAAAGGACCUGCAGGCAGAACUGAUCCUGCUGUUCGAAAAGCAGAAGGAAU |
| | | UCGGAAACCCGCACGUCUCGGGAGGACUGAAGGAAGGAAUCGAAACACUGCUGAUGACACAGAGACCGGCACUGUCGGGAGAC |
| | | GCAGUCCAGAAGAUGCUGGGACACUGCACAUUCGAACCGGCAGAACCGAAGGCAGCAAAGAACACAUACACAGCAGAAAGAUU |
| | | CAUCUGGCUGACAAAGCUGAACAACCUGAGAAUCCUGGAACAGGGAUCGGAAAGACCGCUGACAGACACAGAAAGAGCAACAC |
| | | UGAUGGACGAACCGUACAGAAAGUCGAAGCUGACAUACGCACAGGCAAGAAAGCUGCUGGGACUGGAAGACACAGCAUUCUUC |
| | | AAGGGACUGAGAUACGGAAAGGACAACGCAGAAGCAUCGACACUGAUGGAAAUGAAGGCAUACCACGCAAUCUCGAGAGCACU |
| | | GGAAAAGGAAGGACUGAAGGACAAGAAGUCGCCGCUGAACCUGUCGCCGGAACUGCAGGACGAAAUCGGAACAGCAUUCUCGC |
| | | UGUUCAAGACAGACGAAGACAUCACAGGAAGACUGAAGGACAGAAUCCAGCCGGAAAUCCUGGAAGCACUGCUGAAGCACAUC |
| | | UCGUUCGACAAGUUCGUCCAGAUCUCGCUGAAGGCACUGAGAAGAAUCGUCCCGCUGAUGGAACAGGGAAAGAGAUACGACGA |
| | | AGCAUGCGCAGAAAUCUACGGAGACCACUACGGAAAGAAGAACACAGAAGAAAAGAUCUACCUGCCGCCGAUCCCGGCAGACG |
| | | AAAUCAGAAACCCGGUCGUCCUGAGAGCACUGUCGCAGGCAAGAAAGGUCAUCAACGGAGUCGUCAGAAGAUACGGAUCGCCG |
| | | GCAAGAAUCCACAUCGAAACAGCAAGAGAAGUCGGAAAGUCGUUCAAGGACAGAAAGGAAAUCGAAAAGAGACAGGAAGAAAA |
| | | CAGAAAGGACAGAGAAAAGGCAGCAGCAAAGUUCAGAGAAUACUUCCCGAACUUCGUCGGAGAACCGAAGUCGAAGGACAUCC |
| | | UGAAGCUGAGACUGUACGAACAGCAGCACGGAAAGUGCCUGUACUCGGGAAAGGAAAUCAACCUGGGAAGACUGAACGAAAAG |
| | | GGAUACGUCGAAAUCGACCACGCACUGCCGUUCUCGAGAACAUGGGACGACUCGUUCAACAACAAGGUCCUGGUCCUGGGAUC |
| | | GGAAAACCAGAACAAGGGAAACCAGACACCGUACGAAUACUUCAACGGAAAGGACAACUCGAGAGAAUGGCAGGAAUUCAAGG |
| | | CAAGAGUCGAAACAUCGAGAUUCCCGAGAUCGAAGAAGCAGAGAAUCCUGCUGCAGAAGUUCGACGAAGACGGAUUCAAGGAA |
| | | AGAAACCUGAACGACACAAGAUACGUCAACAGAUUCCUGUGCCAGUUCGUCGCAGACAGAAUGAGACUGACAGGAAAGGGAAA |
| | | GAAGAGAGUCUUCGCAUCGAACGGACAGAUCACAAACCUGCUGAGAGGAUUCUGGGGACUGAGAAAGGUCAGAGCAGAAAACG |
| | | ACAGACACCACGCACUGGACGCAGUCGUCGUCGCAUGCUCGACAGUCGCAAUGCAGCAGAAGAUCACAAGAUUCGUCAGAUAC |
| | | AAGGAAAUGAACGCAUUCGACGGAAAGACAAUCGACAAGGAAACAGGAGAAGUCCUGCACCAGAAGACACACUUCCCGCAGCC |
| | | GUGGGAAUUCUUCGCACAGGAAGUCAUGAUCAGAGUCUUCGGAAAGCCGGACGGAAAGCCGGAAUUCGAAGAAGCAGACACAC |
| | | UGGAAAAGCUGAGAACACUGCUGGCAGAAAAGCUGUCGUCGAGACCGGAAGCAGUCCACGAAUACGUCACACCGCUGUUCGUC |
| | | UCGAGAGCACCGAACAGAAAGAUGUCGGGACAGGGACACAUGGAAACAGUCAAGUCGGCAAAGAGACUGGACGAAGGAGUCUC |
| | | GGUCCUGAGAGUCCCGCUGACACAGCUGAAGCUGAAGGACCUGGAAAAGAUGGUCAACAGAGAAAGAGAACCGAAGCUGUACG |
| | | AAGCACUGAAGGCAAGACUGGAAGCACACAAGGACGACCCGGCAAAGGCAUUCGCAGAACCGUUCUACAAGUACGACAAGGCA |
| | | GGAAACAGAACACAGCAGGUCAAGGCAGUCAGAGUCGAACAGGUCCAGAAGACAGGAGUCUGGGUCAGAAACCACAACGGAAU |
| | | CGCAGACAACGCAACAAUGGUCAGAGUAGACGUCUUCGAAAAGGGAGACAAGUACUACCUGGUCCCGAUCUACUCGUGGCAGG |
| | | UCGCAAAGGGAAUCCUGCCGGACAGAGCAGUCGUCCAGGGAAAGGACGAAGAAGACUGGCAGCUGAUCGACGACUCGUUCAAC |
| | | UUCAAGUUCUCGCUGCACCCGAACGACCUGGUCGAAGUCAUCACAAAGAAGGCAAGAAUGUUCGGAUACUUCGCAUCGUGCCA |
| | | CAGAGGAACAGGAAACAUCAACAUCAGAAUCCACGACCUGGACCACAAGAUCGGAAAGAACGGAAUCCUGGAAGGAAUCGGAG |
| | | UCAAGACAGCACUGUCGUUCCAGAAGUACCAGAUCGACGAACUGGGAAAGGAAAUCAGACCGUGCAGACUGAAGAAGAGACCG |
| | | CCGGUCAGAUCCGGAAAGAGAACAGCAGACGGAUCGGAAUUCGAAUCGCCGAAGAAGAAGAGAAAGGUCGAAUGA |
| Exemplary | 318 | augGACGGCUCCGGCGGCGGCUCCCCCAAGAAGAAGCGGAAGGUGGAGGACAAGCGGCCCGCCGCCACCAAGAAGGCCGGCCA |
| open | | GGCCAAGAAGAAGAAGGGCGGCUCCGGCGGCGGCGCCGCCUUCAAGCCCAACCCCAUCAACUACAUCCUGGGCCUGGACAUCG |
| reading | | GCAUCGCCUCCGUGGGCUGGGCCAUGGUGGAGAUCGACGAGGAGGAGAACCCCAUCCGGCUGAUCGACCUGGGCGUGCGGGUG |
| frame for | | UUCGAGCGGGCCGAGGUGCCCAAGACCGGCGACUCCCUGGCCAUGGCCCGGCGGCUGGCCCGGUCCGUGCGGCGGCUGACCCG |
| Nme2Cas9 | | GCGGCGGGCCCACCGGCUGCUGCGGGCCCGGCGGCUGCUGAAGCGGGAGGGCGUGCUGCAGGCCGCCGACUUCGACGAGAACG |
| with | | GCCUGAUCAAGUCCCUGCCCAACACCCCCUGGCAGCUGCGGGCCGCCGCCCUGGACCGGAAGCUGACCCCCCUGGAGUGGUCC |
| HiBiT tag | | GCCGUGCUGCUGCACCUGAUCAAGCACCGGGGCUACCUGUCCCAGCGGAAGAACGAGGGCGAGACCGCCGACAAGGAGCUGGG |
| | | CGCCCUGCUGAAGGGCGUGGCCAACAACGCCCACGCCCUGCAGACCGGCGACUUCCGGACCCCCGCCGAGCUGGCCCUGAACA |
| | | AGUUCGAGAAGGAGUCCGGCCACAUCCGGAACCAGCGGGGCGACUACUCCCACACCUUCUCCCGGAAGGACCUGCAGGCCGAG |
| | | CUGAUCCUGCUGUUCGAGAAGCAGAAGGAGUUCGGCAACCCCCACGUGUCCGGCGGCCUGAAGGAGGGCAUCGAGACCCUGCU |
| | | GAUGACCCAGCGGCCCGCCCUGUCCGGCGACGCCGUGCAGAAGAUGCUGGGCCACUGCACCUUCGAGCCCGCCGAGCCCAAGG |
| | | CCGCCAAGAACACCUACACCGCCGAGCGGUUCAUCUGGCUGACCAAGCUGAACAACCUGCGGAUCCUGGAGCAGGGCUCCGAG |
| | | CGGCCCCUGACCGACACCGAGCGGGCCACCCUGAUGGACGAGCCCUACCGGAAGUCCAAGCUGACCUACGCCCAGGCCCGGAA |
| | | GCUGCUGGGCCUGGAGGACACCGCCUUCUUCAAGGGCCUGCGGUACGGCAAGGACAACGCCGAGGCCUCCACCCUGAUGGAGA |
| | | UGAAGGCCUACCACGCCAUCUCCCGGGCCCUGGAGAAGGAGGGCCUGAAGGACAAGAAGUCCCCCCUGAACCUGUCCUCCGAG |
| | | CUGCAGGACGAGAUCGGCACCGCCUUCUCCCUGUUCAAGACCGACGAGGACAUCACCGGCCGGCUGAAGGACCGGGUGCAGCC |
| | | CGAGAUCCUGGAGGCCCUGCUGAAGCACAUCUCCUUCGACAAGUUCGUGCAGAUCUCCCUGAAGGCCCUGCGGCGGAUCGUGC |
| | | CCCUGAUGGAGCAGGGCAAGCGGUACGACGAGGCCUGCGCCGAGAUCUACGGCGACCACUACGGCAAGAAGAACACCGAGGAG |
| | | AAGAUCUACCUGCCCCCCAUCCCCGCCGACGAGAUCCGGAACCCCGUGGUGCUGCGGGCCCUGUCCCAGGCCCGGAAGGUGAU |
| | | CAACGGCGUGGUGCGGCGGUACGGCUCCCCCGCCCGGAUCCACAUCGAGACCGCCCGGGAGGUGGGCAAGUCCUUCAAGGACC |
| | | GGAAGGAGAUCGAGAAGCGGCAGGAGGAGAACCGGAAGGACCGGGAGAAGGCCGCCGCCAAGUUCCGGGAGUACUUCCCCAAC |
| | | UUCGUGGGCGAGCCCAAGUCCAAGGACAUCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCCCUGCCCUUCUCCCGGACCUGGGACGACU |
| | | CCUUCAACAACAAGGUGCUGGUGCUGGGCUCCGAGAACCAGAACAAGGGCAACCAGACCCCCUACGAGUACUUCAACGGCAAG |
| | | GACAACUCCCGGGAGUGGCAGGAGUUCAAGGCCCGGGUGGAGACCUCCCGGUUCCCCCGGUCCAAGAAGCAGCGGAUCCUGCU |
| | | GCAGAAGUUCGACGAGGACGGCUUCAAGGAGUGCAACCUGAACGACACCCGGUACGUGAACCGGUUCCUGUGCCAGUUCGUGG |
| | | CCGACCACAUCCUGCUGACCGGCAAGGGCAAGCGGCGGGUGUUCGCCUCCAACGGCCAGAUCACCAACCUGCUGCGGGGCUUC |
| | | UGGGGCCUGCGGAAGGUGCGGGCCGAGAACGACCGGCACCACGCCCUGGACGCCGUGGUGGUGGCCUGCUCCACCGUGGCCAU |
| | | GCAGCAGAAGAUCACCCGGUUCGUGCGGUACAAGGAGAUGAACGCCUUCGACGGCAAGACCAUCGACAAGGAGACCGGCAAGG |
| | | UGCUGCACCAGAAGACCCACUUCCCCCAGCCCUGGGAGUUCUUCGCCCAGGAGGUGAUGAUCCGGGUGUUCGGCAAGCCCGAC |
| | | GGCAAGCCCGAGUUCGAGGAGGCCGACACCCCCGAGAAGCUGCGGACCCUGCUGGCCGAGAAGCUGUCCUCCCGGCCCGAGGC |
| | | CGUGCACGAGUACGUGACCCCCCUGUUCGUGUCCCGGGCCCCCAACCGGAAGAUGUCCGGCGCCCACAAGGACACCCUGCGGU |
| | | CCGCCAAGCGGUUCGUGAAGCACAACGAGAAGAUCUCCGUGAAGCGGGUGUGGCUGACCGAGAUCAAGCUGGCCGACCUGGAG |
| | | AACAUGGUGAACUACAAGAACGGCCGGGAGAUCGAGCUGUACGAGGCCCUGAAGGCCCGGCUGGAGGCCUACGGCGGCAACGC |
| | | CAAGCAGGCCUUCGACCCCAAGGACAACCCCUUCUACAAGAAGGGCGGCCAGCUGGUGAAGGCCGUGCGGGUGGAGAAGACCC |
| | | AGGAGUCCGGCGUGCUGCUGAACAAGAAGAACGCCUACACCAUCGCCGACAACGGCGACAUGGUGCGGGUGGACGUGUUCUGC |
| | | AAGGUGGACAAGAAGGGCAAGAACCAGUACUUCAUCGUGCCCAUCUACGCCUGGCAGGUGGCCGAGAACAUCCUGCCCGACAU |
| | | CGACUGCAAGGGCUACCGGAUCGACGACUCCUACACCUUCUGCUUCUCCCUGCACAAGUACGACCUGAUCGCCUUCCAGAAGG |
| | | ACGAGAAGUCCAAGGUGGAGUUCGCCUACUACAUCAACUGCGACUCCUCCAACGGCCGGUUCUACCUGGCCUGGCACGACAAG |
| | | GGCUCCAAGGAGCAGCAGUUCCGGAUCUCCACCCAGAACCUGGUGCUGAUCCAGAAGUACCAGGUGAACGAGCUGGGCAAGGA |
| | | GAUCCGGCCCUGCCGGCUGAAGAAGCGGCCCCCCGUGCGGUCCGAGUCCGCCACCCCCGAGUCCGUGUCCGGCUGGCGGCUGU |
| | | UCAAGAAGAUCUCCUAG |
| Exemplary | 319 | augGACGGCUCCGGCGGCGGCUCCCCCAAGAAGAAGCGGAAGGUGGAGGACAAGCGGCCCGCCGCCACCAAGAAGGCCGGCCA |
| open | | GGCCAAGAAGAAGAAGGGCGGCUCCGGCGGCGGCGCAGCAUUCAAGCCAAACUCAAUCAAUUACAUCCUGGGACUGGACAUCG |
| reading | | GCAUCGCAUCCGUCGGGUGGGCUAUGGUCGAAAUCGACGAGGAGGAGAACCCCAUCCGCCUGAUCGAUCUGGGCGUGCGCGUG |
| frame for | | UUUGAGAGGGCAGAGGUGCCUAAGACCGGCGACAGCCUGGCCAUGGCACGGAGACUGGCACGCUCCGUGAGGCGCCUGACCCG |
| Nme1Cas9 | | GAGAAGGGCCCACAGACUGCUGAGGACACGCCGGCUGCUGAAGAGGGAGGGCGUGCUGCAGGCCGCCAACUUCGAUGAGAAUG |
| with | | GCCUGAUCAAGUCCCUGCCCAAUACCCCUUGGCAGCUGAGGGCAGCCGCCCUGGACCGCAAGCUGACACCUCUGGAGUGGUCC |
| HiBiT tag | | GCCGUGCUGCUGCACCUGAUCAAGCACCGGGGCUACCUGUCUCAGAGAAAGAACGAGGGCGAGACAGCCGAUAAGGAGCUGGG |
| | | CGCCCUGCUGAAGGGAGUGGCAGGAAAUGCACACGCCCUGCAGACCGGCGACUUUCGCACACCAGCCGAGCUGGCCCUGAACA |
| | | AGUUCGAGAAGGAGAGCGGCCACAUCCGCAAUCAGCGGUCUGACUAUAGCCACACCUUCUCCCGGAAGGAUCUGCAGGCCGAG |
| | | CUGAUCCUGCUGUUUGAGAAGCAGAAGGAGUUCGGCAACCCACACGUGUCUGGCGGCCUGAAGGAGGGCAUCGAGACACUGCU |
| | | GAUGACACAGCGGCCCGCCCUGAGCGGCGACGCAGUGCAGAAGAUGCUGGGACACUGCACCUUUGAGCCAGCCGAGCCCAAGG |
| | | CCGCCAAGAAUACCUACACAGCCGAGCGGUUCAUCUGGCUGACAAAGCUGAACAAUCUGAGGAUCCUGGAGCAGGGAAGCGAG |
| | | CGCCCACUGACCGACACAGAGAGGGCCACCCUGAUGGAUGAGCCCUACCGCAAGUCCAAGCUGACAUAUGCACAGGCAAGGAA |
| | | GCUGCUGGGCCUGGAGGACACCGCCUUCUUUAAGGGCCUGAGAUACGGCAAGGAUAACGCCGAGGCCUCUACACUGAUGGAGA |
| | | UGAAGGCCUAUCACGCCAUCAGCAGGGCCCUGGAGAAGGAGGGCCUGAAGGACAAGAAGUCCCCACUGAAUCUGUCUCCCGAG |
| | | CUGCAGGAUGAGAUCGGCACCGCCUUUAGCCUGUUCAAGACCGACGAGGAUAUCACAGGCAGACUGAAGGACAGGAUCCAGCC |
| | | AGAGAUCCUGGAGGCCCUGCUGAAGCACAUCAGCUUUGAUAAGUUCGUGCAGAUCAGCCUGAAGGCCCUGCGGAGGAUCGUGC |
| | | CACUGAUGGAGCAGGGCAAGAGGUACGACGAGGCCUGCGCCGAAAUCUACGGCGAUCACUAUGGCAAGAAGAACACAGAGGAG |
| | | AAAAUCUACCUGCCCCCUAUCCCCGCCGAUGAGAUCAGGAACCCUGUGGUGCUGCGCGCCCUGUCUCAGGCAAGAAAAGUGAU |
| | | CAACGGAGUGGUGCGCCGGUACGGCAGCCCCGCCAGAAUCCACAUCGAGACAGCCAGGGAAGUGGGCAAGUCCUUUAAGGACA |
| | | GAAAGGAGAUCGAGAAGAGGCAGGAGGAGAACAGAAAGGAUAGGGAGAAGGCCGCCGCCAAGUUCAGAGAGUACUUUCCUAAU |
| | | UUCGUGGGCGAGCCAAAGUCCAAGGAUAUCCUGAAGCUGAGGCUGUACGAGCAGCAGCACGGCAAGUGUCUGUAUUCUGGCAA |
| | | GGAGAUCAACCUGGGCCGCCUGAAUGAGAAGGGCUAUGUGGAGAUCGACCACGCCCUGCCUUUUUCUCGGACCUGGGACGAUA |
| | | GCUUCAACAAUAAGGUGCUGGUGCUGGGCUCUGAGAACCAGAAUAAGGGCAACCAGACACCCUACGAGUAUUUCAACGGCAAG |
| | | GACAAUAGCCGCGAGUGGCAGGAGUUUAAGGCAAGGGUGGAGACAAGCAGGUUCCCUCGGUCCAAGAAGCAGAGAAUCCUGCU |
| | | GCAGAAGUUUGACGAGGAUGGCUUCAAGGAGAGGAACCUGAAUGACACCCGCUACGUGAAUCGGUUUCUGUGCCAGUUCGUGG |
| | | CCGAUAGAAUGAGGCUGACCGGCAAGGGCAAGAAGAGAGUGUUUGCCUCCAACGGCCAGAUCACAAAUCUGCUGAGGGGCUUC |
| | | UGGGGCCUGAGAAAGGUGAGGGCAGAGAACGACAGGCACCACGCACUGGAUGCAGUGGUGGUGGCAUGUUCUACCGUGGCCAU |
| | | GCAGCAGAAGAUCACACGCUUUGUGCGGUAUAAGGAGAUGAAUGCCUUCGACGGCAAGACCAUCGAUAAGGAGACAGGCGAGG |
| | | UGCUGCACCAGAAGACACACUUUCCUCAGCCAUGGGAGUUCUUUGCCCAGGAAGUGAUGAUCCGGGUGUUUGGCAAGCCUGAC |
| | | GGCAAGCCAGAGUUCGAGGAGGCCGAUACCCUGGAGAAGCUGAGAACACUGCUGGCAGAGAAGCUGAGCUCCAGGCCCGAGGC |
| | | AGUGCACGAGUACGUGACCCCACUGUUCGUGUCUAGAGCCCCCAACAGGAAGAUGAGCGGCCAGGGCCACAUGGAGACAGUGA |
| | | AGUCCGCCAAGAGACUGGACGAGGGCGUGUCUGUGCUGAGGGUGCCUCUGACACAGCUGAAGCUGAAGGAUCUGGAGAAGAUG |
| | | GUGAAUCGCGAGCGGGAGCCAAAGCUGUAUGAGGCCCUGAAGGCAAGGCUGGAGGCACACAAGGACGAUCCUGCCAAGGCCUU |
| | | UGCCGAGCCAUUCUACAAGUAUGAUAAGGCCGGCAACAGAACCCAGCAGGUGAAGGCCGUGAGGGUGGAGCAGGUGCAGAAGA |
| | | CAGGCGUGUGGGUGCGCAACCACAAUGGCAUCGCCGACAAUGCUACCAUGGUGCGGGUGGACGUGUUUGAGAAGGGCGAUAAG |
| | | UACUAUCUGGUGCCCAUCUACAGCUGGCAGGUGGCCAAGGGCAUCCUGCCUGAUAGAGCCGUGGUGCAGGGCAAGGACGAGGA |
| | | GGAUUGGCAGCUGAUCGACGAUUCCUUCAACUUUAAGUUCUCUCUGCACCCCAAUGACCUGGUGGAAGUGAUCACCAAGAAGG |
| | | CCAGGAUGUUUGGCUACUUCGCCUCCUGCCACCGCGGCACAGGCAACAUCAAUAUCCGGAUCCACGACCUGGAUCACAAGAUC |
| | | GGCAAGAACGGCAUCCUGGAGGGCAUCGGCGUGAAGACAGCCCUGAGCUUCCAGAAGUAUCAGAUCGACGAGCUGGGCAAGGA |
| | | GAUCAGACCUUGUAGGCUGAAGAAGCGCCCACCCGUGCGGUCCGAGUCCGCCACCCCCGAGUCCGUGUCCGGCUGGCGGCUGU |
| | | UCAAGAAGAUCUCCUAG |
| Exemplary | 320 | augGACGGCUCCGGCGGCGGCUCCCCCAAGAAGAAGCGGAAGGUGGAGGACAAGCGGCCCGCCGCCACCAAGAAGGCCGGCCA |
| open | | GGCCAAGAAGAAGAAGGGCGGCUCCGGCGGCGGCGCCGCCUUCAAGCCCAACUCCAUCAACUACAUCCUGGGCCUGGACAUCG |
| reading | | GCAUCGCCUCCGUGGGCUGGGCCAUGGUGGAGAUCGACGAGGAGGAGAACCCCAUCCGGCUGAUCGACCUGGGCGUGCGGGUG |
| frame for | | UUCGAGCGGGCCGAGGUGCCCAAGACCGGCGACUCCCUGGCCAUGGCCCGGCGGCUGGCCCGGUCCGUGCGGCGGCUGACCCG |
| Nme1Cas9 | | GCGGCGGGCCCACCGGCUGCUGCGGACCCGGCGGCUGCUGAAGCGGGAGGGCGUGCUGCAGGCCGCCAACUUCGACGAGAACG |
| with | | GCCUGAUCAAGUCCCUGCCCAACACCCCCUGGCAGCUGCGGGCCGCCGCCCUGGACCGGAAGCUGACCCCCCUGGAGUGGUCC |
| HiBiT tag | | GCCGUGCUGCUGCACCUGAUCAAGCACCGGGGCUACCUGUCCCAGCGGAAGAACGAGGGCGAGACCGCCGACAAGGAGCUGGG |
| | | CGCCCUGCUGAAGGGCGUGGCCGGCAACGCCCACGCCCUGCAGACCGGCGACUUCCGGACCCCCGCCGAGCUGGCCCUGAACA |
| | | AGUUCGAGAAGGAGUCCGGCCACAUCCGGAACCAGCGGUCCGACUACUCCCACACCUUCUCCCGGAAGGACCUGCAGGCCGAG |
| | | CUGAUCCUGCUGUUCGAGAAGCAGAAGGAGUUCGGCAACCCCCACGUGUCCGGCGGCCUGAAGGAGGGCAUCGAGACCCUGCU |
| | | GAUGACCCAGCGGCCCGCCCUGUCCGGCGACGCCGUGCAGAAGAUGCUGGGCCACUGCACCUUCGAGCCCGCCGAGCCCAAGG |
| | | CCGCCAAGAACACCUACACCGCCGAGCGGUUCAUCUGGCUGACCAAGCUGAACAACCUGCGGAUCCUGGAGCAGGGCUCCGAG |
| | | CGGCCCCUGACCGACACCGAGCGGGCCACCCUGAUGGACGAGCCCUACCGGAAGUCCAAGCUGACCUACGCCCAGGCCCGGAA |
| | | GCUGCUGGGCCUGGAGGACACCGCCUUCUUCAAGGGCCUGCGGUACGGCAAGGACAACGCCGAGGCCUCCACCCUGAUGGAGA |
| | | UGAAGGCCUACCACGCCAUCUCCCGGGCCCUGGAGAAGGAGGGCCUGAAGGACAAGAAGUCCCCCCUGAACCUGUCCCCCGAG |
| | | CUGCAGGACGAGAUCGGCACCGCCUUCUCCCUGUUCAAGACCGACGAGGACAUCACCGGCCGGCUGAAGGACCGGAUCCAGCC |
| | | CGAGAUCCUGGAGGCCCUGCUGAAGCACAUCUCCUUCGACAAGUUCGUGCAGAUCUCCCUGAAGGCCCUGCGGCGGAUCGUGC |
| | | CCCUGAUGGAGCAGGGCAAGCGGUACGACGAGGCCUGCGCCGAGAUCUACGGCGACCACUACGGCAAGAAGAACACCGAGGAG |
| | | AAGAUCUACCUGCCCCCCAUCCCCGCCGACGAGAUCCGGAACCCCGUGGUGCUGCGGGCCCUGUCCCAGGCCCGGAAGGUGAU |
| | | CAACGGCGUGGUGCGGCGGUACGGCUCCCCCGCCCGGAUCCACAUCGAGACCGCCCGGGAGGUGGGCAAGUCCUUCAAGGACC |
| | | GGAAGGAGAUCGAGAAGCGGCAGGAGGAGAACCGGAAGGACCGGGAGAAGGCCGCCGCCAAGUUCCGGGAGUACUUCCCCAAC |
| | | UUCGUGGGCGAGCCCAAGUCCAAGGACAUCCUGAAGCUGCGGCUGUACGAGCAGCAGCACGGCAAGUGCCUGUACUCCGGCAA |
| | | GGAGAUCAACCUGGGCCGGCUGAACGAGAAGGGCUACGUGGAGAUCGACCACGCCCUGCCCUUCUCCCGGACCUGGGACGACU |
| | | CCUUCAACAACAAGGUGCUGGUGCUGGGCUCCGAGAACCAGAACAAGGGCAACCAGACCCCCUACGAGUACUUCAACGGCAAG |
| | | GACAACUCCCGGGAGUGGCAGGAGUUCAAGGCCCGGGUGGAGACCUCCCGGUUCCCCCGGUCCAAGAAGCAGCGGAUCCUGCU |
| | | GCAGAAGUUCGACGAGGACGGCUUCAAGGAGCGGAACCUGAACGACACCCGGUACGUGAACCGGUUCCUGUGCCAGUUCGUGG |
| | | CCGACCGGAUGCGGCUGACCGGCAAGGGCAAGAAGCGGGUGUUCGCCUCCAACGGCCAGAUCACCAACCUGCUGCGGGGCUUC |
| | | UGGGGCCUGCGGAAGGUGCGGGCCGAGAACGACCGGCACCACGCCCUGGACGCCGUGGUGGUGGCCUGCUCCACCGUGGCCAU |
| | | GCAGCAGAAGAUCACCCGGUUCGUGCGGUACAAGGAGAUGAACGCCUUCGACGGCAAGACCAUCGACAAGGAGACCGGCGAGG |
| | | UGCUGCACCAGAAGACCCACUUCCCCCAGCCCUGGGAGUUCUUCGCCCAGGAGGUGAUGAUCCGGGUGUUCGGCAAGCCCGAC |
| | | GGCAAGCCCGAGUUCGAGGAGGCCGACACCCUGGAGAAGCUGCGGACCCUGCUGGCCGAGAAGCUGUCCUCCCGGCCCGAGGC |
| | | CGUGCACGAGUACGUGACCCCCCUGUUCGUGUCCCGGGCCCCCAACCGGAAGAUGUCCGGCCAGGGCCACAUGGAGACCGUGA |
| | | AGUCCGCCAAGCGGCUGGACGAGGGCGUGUCCGUGCUGCGGGUGCCCCUGACCCAGCUGAAGCUGAAGGACCUGGAGAAGAUG |
| | | GUGAACCGGGAGCGGGAGCCCAAGCUGUACGAGGCCCUGAAGGCCCGGCUGGAGGCCCACAAGGACGACCCCGCCAAGGCCUU |
| | | CGCCGAGCCCUUCUACAAGUACGACAAGGCCGGCAACCGGACCCAGCAGGUGAAGGCCGUGCGGGUGGAGCAGGUGCAGAAGA |
| | | CCGGCGUGUGGGUGCGGAACCACAACGGCAUCGCCGACAACGCCACCAUGGUGCGGGUGGACGUGUUCGAGAAGGGCGACAAG |
| | | UACUACCUGGUGCCCAUCUACUCCUGGCAGGUGGCCAAGGGCAUCCUGCCCGACCGGGCCGUGGUGCAGGGCAAGGACGAGGA |
| | | GGACUGGCAGCUGAUCGACGACUCCUUCAACUUCAAGUUCUCCCUGCACCCCAACGACCUGGUGGAGGUGAUCACCAAGAAGG |
| | | CCCGGAUGUUCGGCUACUUCGCCUCCUGCCACCGGGGCACCGGCAACAUCAACAUCCGGAUCCACGACCUGGACCACAAGAUC |
| | | GGCAAGAACGGCAUCCUGGAGGGCAUCGGCGUGAAGACCGCCCUGUCCUUCCAGAAGUACCAGAUCGACGAGCUGGGCAAGGA |
| | | GAUCCGGCCCUGCCGGCUGAAGAAGCGGCCCCCCGUGCGGUCCGAGUCCGCCACCCCCGAGUCCGUGUCCGGCUGGCGGCUGU |
| | | UCAAGAAGAUCUCCUAG |
| Exemplary | 321 | augGACGGCUCCGGCGGCGGCUCCCCCAAGAAGAAGCGGAAGGUGGAGGACAAGCGGCCCGCCGCCACCAAGAAGGCCGGCCA |
| open | | GGCCAAGAAGAAGAAGGGCGGCUCCGGCGGCGCCGCCUUCAAGCCCAACCCCAUCAACUACAUCCUGGGCCUGGACAUCGGCA |
| reading | | UCGCCUCCGUGGGCUGGGCCAUGGUGGAGAUCGACGAGGAGGAGAACCCCAUCCGGCUGAUCGACCUGGGCGUGCGGGUGUUC |
| frame for | | GAGCGGGCCGAGGUGCCCAAGACCGGCGACUCCCUGGCCAUGGCCCGGCGGCUGGCCCGGUCCGUGCGGCGGCUGACCCGGCG |
| Nme3Cas9 | | GCGGGCCCACCGGCUGCUGCGGGCCCGGCGGCUGCUGAAGCGGGAGGGCGUGCUGCAGGCCGCCGACUUCGACGAGAACGGCC |
| with | | UGAUCAAGUCCCUGCCCAACACCCCCUGGCAGCUGCGGGCCGCCGCCCUGGACCGGAAGCUGACCCCCCUGGAGUGGUCCGCC |
| HiBiT tag | | GUGCUGCUGCACCUGAUCAAGCACCGGGGCUACCUGUCCCAGCGGAAGAACGAGGGCGAGACCGCCGACAAGGAGCUGGGCGC |
| | | CCUGCUGAAGGGCGUGGCCGACAACGCCCACGCCCUGCAGACCGGCGACUUCCGGACCCCCGCCGAGCUGGCCCUGAACAAGU |
| | | UCGAGAAGGAGUGCGGCCACAUCCGGAACCAGCGGGGCGACUACUCCCACACCUUCUCCCGGAAGGACCUGCAGGCCGAGCUG |
| | | AACCUGCUGUUCGAGAAGCAGAAGGAGUUCGGCAACCCCCACGUGUCCGGCGGCCUGAAGGAGGGCAUCGAGACCCUGCUGAU |
| | | GACCCAGCGGCCCGCCCUGUCCGGCGACGCCGUGCAGAAGAUGCUGGGCCACUGCACCUUCGAGCCCGCCGAGCCCAAGGCCG |
| | | CCAAGAACACCUACACCGCCGAGCGGUUCAUCUGGCUGACCAAGCUGAACAACCUGCGGAUCCUGGAGCAGGGCUCCGAGCGG |
| | | CCCCUGACCGACACCGAGCGGGCCACCCUGAUGGACGAGCCCUACCGGAAGUCCAAGCUGACCUACGCCCAGGCCCGGAAGCU |
| | | GCUGUCCCUGGAGGACACCGCCUUCUUCAAGGGCCUGCGGUACGGCAAGGACAACGCCGAGGCCUCCACCCUGAUGGAGAUGA |
| | | AGGCCUACCACACCAUCUCCCGGGCCCUGGAGAAGGAGGGCCUGAAGGACAAGAAGUCCCCCCUGAACCUGUCCCCCGAGCUG |
| | | CAGGACGAGAUCGGCACCGCCUUCUCCCUGUUCAAGACCGACGAGGACAUCACCGGCCGGCUGAAGGACCGGAUCCAGCCCGA |
| | | GAUCCUGGAGGCCCUGCUGAAGCACAUCUCCUUCGACAAGUUCGUGCAGAUCUCCCUGAAGGCCCUGCGGCGGAUCGUGCCCC |
| | | UGAUGGAGCAGGGCAAGCGGUACGACGAGGCCUGCGCCGAGAUCUACGGCGACCACUACGGCAAGAAGAACACCGAGGAGAAG |
| | | AUCUACCUGCCCCCCAUCCCCGCCGACGAGAUCCGGAACCCCGUGGUGCUGCGGGCCCUGUCCCAGGCCCGGAAGGUGAUCAA |
| | | CGGCGUGGUGCGGCGGUACGGCUCCCCCGCCCGGAUCCACAUCGAGACCGCCCGGGAGGUGGGCAAGUCCUUCAAGGACCGGA |
| | | AGGAGAUCGAGAAGCGGCAGGAGGAGAACCGGAAGGACCGGGAGAAGGCCGCCGCCAAGUUCCGGGAGUACUUCCCCAACUUC |
| | | GUGGGCGAGCCCAAGUCCAAGGACAUCCUGAAGCUGCGGCUGUACGAGCAGCAGCACGGCAAGUGCCUGUACUCCGGCAAGGA |
| | | GAUCAACCUGGGCCGGCUGAACGAGAAGGGCUACGUGGAGAUCGACCACGCCCUGCCCUUCUCCCGGACCUGGGACGACUCCU |
| | | UCAACAACAAGGUGCUGGUGCUGGGCUCCGAGAACCAGAACAAGGGCAACCAGACCCCCUACGAGUACUUCAACGGCAAGGAC |
| | | AACUCCCGGGAGUGGCAGGAGUUCAAGGCCCGGGUGGAGACCUCCCGGUUCCCCCGGUCCAAGAAGCAGCGGAUCCUGCUGCA |
| | | GAAGUUCGACGAGGACGGCUUCAAGGAGCGGAACCUGAACGACACCCGGUACGUGAACCGGUUCCUGUGCCAGUUCGUGGCCG |
| | | ACCGGAUGCGGCUGACCGGCAAGGGCAAGAAGCGGGUGUUCGCCUCCAACGGCCAGAUCACCAACCUGCUGCGGGGCUUCUGG |
| | | GGCCUGCGGAAGGUGCGGGCCGAGAACGACCGGCACCACGCCCUGGACGCCGUGGUGGUGGCCUGCUCCACCGUGGCCAUGCA |
| | | GCAGAAGAUCACCCGGUUCGUGCGGUACAAGGAGAUGAACGCCUUCGACGGCAAGACCAUCGACAAGGAGACCGGCGAGGUGC |
| | | UGCACCAGAAGACCCACUUCCCCCAGCCCUGGGAGUUCUUCGCCCAGGAGGUGAUGAUCCGGGUGUUCGGCAAGCCCGACGGC |
| | | AAGCCCGAGUUCGAGGAGGCCGACACCCCCGAGAAGCUGCGGACCCUGCUGGCCGAGAAGCUGUCCUCCCGGCCCGAGGCCGU |
| | | GCACGAGUACGUGACCCCCCUGUUCGUGUCCCGGGCCCCCAACCGGAAGAUGUCCGGCCAGGGCCACAUGGAGACCGUGAAGU |
| | | CCGCCAAGCGGCUGGACGAGGGCGUGUCCGUGCUGCGGGUGCCCCUGACCCAGCUGAAGCUGAAGGACCUGGAGAAGAUGGUG |
| | | AACCGGGAGCGGGAGCCCAAGCUGUACGAGGCCCUGAAGGCCCGGCUGGAGGCCCACAAGGACGACCCCGCCAAGGCCUUCGC |
| | | CGAGCCCUUCUACAAGUACGACAAGGCCGGCAACCGGACCCAGCAGGUGAAGGCCGUGCGGGUGGAGCAGGUGCAGAAGACCG |
| | | GCGUGUGGGUGCGGAACCACAACGGCAUCGCCGACAACGCCACCAUGGUGCGGGUGGACGUGUUCGAGAAGGGCGACAAGUAC |
| | | UACCUGGUGCCCAUCUACUCCUGGCAGGUGGCCAAGGGCAUCCUGCCCGACCGGGCCGUGGUGGCCUACGCCGACGAGGAGGA |
| | | CUGGACCGUGAUCGACGAGUCCUUCCGGUUCAAGUUCGUGCUGUACUCCAACGACCUGAUCAAGGUGCAGCUGAAGAAGGACU |
| | | CCUUCCUGGGCUACUUCUCCGGCCUGGACCGGGCCACCGGCGCCAUCUCCCUGCGGGAGCACGACCUGGAGAAGUCCAAGGGC |
| | | AAGGACGGCAUGCACCGGAUCGGCGUGAAGACCGCCCUGUCCUUCCAGAAGUACCAGAUCGACGAGAUGGGCAAGGAGAUCCG |
| | | GCCCUGCCGGCUGAAGAAGCGGCCCCCCGUGCGGUCCGAGUCCGCCACCCCCGAGUCCGUGUCCGGCUGGCGGCUGUUCAAGA |
| | | AGAUCUCCUAG |

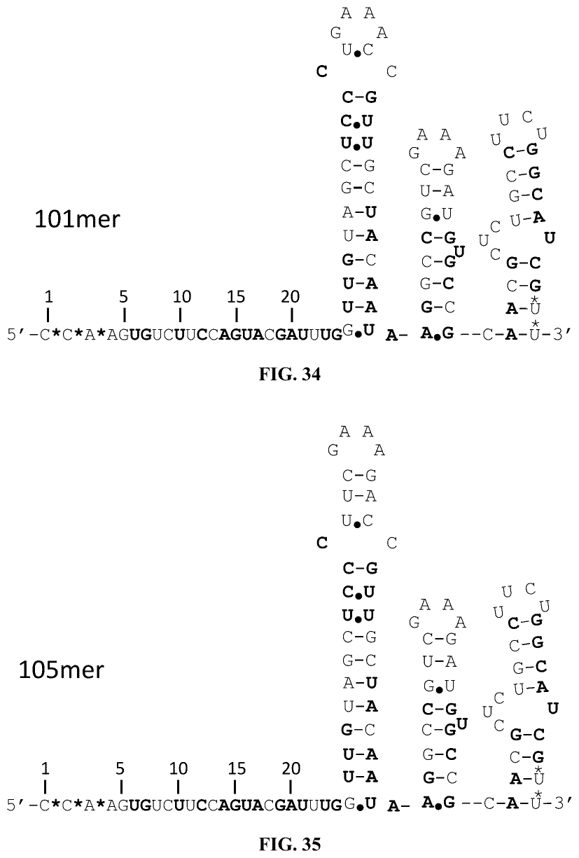

TABLE 39B
Additional Sequences

| | SEQ | |
|---|---|---|
| | ID | |
| Description | NO | sequence |
| exemplary XTEN | 58 | SGSETPGTSESATPES |
| exemplary XTEN | 59 | SGSETPGTSESA |
| exemplary XTEN | 60 | SGSETPGTSESATPEGGSGGS |
| amino acid sequence for exemplary | 61 | GGS |
| linker | | |
| amino acid sequence for exemplary | 62 | GGGGS |
| linker | | |
| amino acid sequence for exemplary | 63 | EAAAK |
| linker | | |
| amino acid sequence for exemplary | 64 | SEGSA |
| linker | | |
| amino acid sequence for exemplary | 65 | SEGSAGTST |
| linker | | |
| amino acid sequence for exemplary | 66 | GGGGSGGGGS |
| linker | | |
| amino acid sequence for exemplary | 67 | GGGGSEAAAK |
| linker | | |
| amino acid sequence for exemplary | 68 | EAAAKGGGGS |
| linker | | |
| amino acid sequence for exemplary | 69 | EAAAKEAAAK |
| linker | | |
| amino acid sequence for exemplary | 70 | SEGSAGTSTESEGSA |
| linker | | |
| amino acid sequence for exemplary | 71 | GGGGSGGGGSGGGGS |
| linker | | |
| amino acid sequence for exemplary | 72 | GGGGSGGGGSEAAAK |
| linker | | |
| amino acid sequence for exemplary | 73 | GGGGSEAAAKGGGGS |
| linker | | |
| amino acid sequence for exemplary | 74 | EAAAKGGGGSEAAAK |
| linker | | |
| amino acid sequence for exemplary | 75 | EAAAKEAAAKGGGGS |
| linker | | |
| amino acid sequence for exemplary | 76 | SEGSAGTSTESEGSAGTSTE |
| linker | | |
| amino acid sequence for exemplary | 77 | GGGGSGGGGSGGGGSEAAAK |
| linker | | |
| amino acid sequence for exemplary | 78 | GGGGSGGGGSEAAAKGGGGS |
| linker | | |
| amino acid sequence for exemplary | 79 | GGGGSEAAAKGGGGSEAAAK |
| linker | | |
| amino acid sequence for exemplary | 80 | GGGGSEAAAKEAAAKGGGGS |
| linker | | |
| amino acid sequence for exemplary | 81 | GGGGSEAAAKEAAAKEAAAK |
| linker | | |
| amino acid sequence for exemplary | 82 | EAAAKGGGGSGGGGSGGGGS |
| linker | | |
| amino acid sequence for exemplary | 83 | EAAAKGGGGSGGGGSEAAAK |
| linker | | |
| amino acid sequence for exemplary | 84 | EAAAKGGGGSEAAAKGGGGS |
| linker | | |
| amino acid sequence for exemplary | 85 | EAAAKGGGGSEAAAKEAAAK |
| linker | | |
| amino acid sequence for exemplary | 86 | EAAAKEAAAKGGGGSGGGGS |
| linker | | |
| amino acid sequence for exemplary | 87 | EAAAKEAAAKGGGGSEAAAK |
| linker | | |
| amino acid sequence for exemplary | 88 | EAAAKEAAAKEAAAKGGGGS |
| linker | | |
| amino acid sequence for exemplary | 89 | SEGSAGTSTESEGSAGTSTESEGSA |
| linker | | |
| amino acid sequence for exemplary | 90 | GGGGSGGGGSGGGGSGGGGSGGGGS |
| linker | | |
| amino acid sequence for exemplary | 91 | GGGGSGGGGSGGGGSGGGGSEAAAK |
| linker | | |
| amino acid sequence for exemplary | 92 | GGGGSGGGGSGGGGSEAAAKGGGGS |
| linker | | |
| amino acid sequence for exemplary | 93 | GGGGSGGGGSGGGGSEAAAKEAAAK |
| linker | | |
| amino acid sequence for exemplary | 94 | GGGGSGGGGSEAAAKGGGGSGGGGS |
| linker | | |
| amino acid sequence for exemplary | 95 | GGGGSGGGGSEAAAKGGGGSEAAAK |
| linker | | |
| amino acid sequence for exemplary | 96 | GGGGSGGGGSEAAAKEAAAKGGGGS |
| linker | | |
| amino acid sequence for exemplary | 97 | GGGGSGGGGSEAAAKEAAAKEAAAK |
| linker | | |
| amino acid sequence for exemplary | 98 | GGGGSEAAAKGGGGSGGGGSGGGGS |
| linker | | |
| amino acid sequence for exemplary | 99 | GGGGSEAAAKGGGGSGGGGSEAAAK |
| linker | | |
| amino acid sequence for exemplary | 100 | GGGGSEAAAKGGGGSEAAAKGGGGS |
| linker | | |
| amino acid sequence for exemplary | 101 | GGGGSEAAAKGGGGSEAAAKEAAAK |
| linker | | |
| amino acid sequence for exemplary | 102 | GGGGSEAAAKEAAAKGGGGSGGGGS |
| linker | | |
| amino acid sequence for exemplary | 103 | GGGGSEAAAKEAAAKEAAAKGGGGS |
| linker | | |
| amino acid sequence for exemplary | 104 | GGGGSEAAAKEAAAKEAAAKEAAAK |
| linker | | |
| amino acid sequence for exemplary | 105 | EAAAKGGGGSGGGGSGGGGSGGGGS |
| linker | | |
| amino acid sequence for exemplary | 106 | EAAAKGGGGSGGGGSGGGGSEAAAK |
| linker | | |
| amino acid sequence for exemplary | 107 | EAAAKGGGGSGGGGSEAAAKGGGGS |
| linker | | |
| amino acid sequence for exemplary | 108 | EAAAKGGGGSGGGGSEAAAKEAAAK |
| linker | | |
| amino acid sequence for exemplary | 109 | EAAAKGGGGSEAAAKGGGGSGGGGS |
| linker | | |
| amino acid sequence for exemplary | 110 | EAAAKGGGGSEAAAKGGGGSEAAAK |
| linker | | |
| amino acid sequence for exemplary | 111 | EAAAKGGGGSEAAAKEAAAKGGGGS |
| linker | | |
| amino acid sequence for exemplary | 112 | EAAAKGGGGSEAAAKEAAAKEAAAK |
| linker | | |
| amino acid sequence for exemplary | 113 | EAAAKEAAAKGGGGSEAAAKGGGGS |
| linker | | |
| amino acid sequence for exemplary | 114 | EAAAKEAAAKGGGGSEAAAKEAAAK |
| linker | | |
| amino acid sequence for exemplary | 115 | EAAAKEAAAKEAAAKGGGGSEAAAK |
| linker | | |
| amino acid sequence for exemplary | 116 | EAAAKEAAAKEAAAKEAAAKGGGGS |
| linker | | |
| amino acid sequence for exemplary | 117 | EAAAKEAAAKEAAAKEAAAKEAAAK |
| linker | | |
| amino acid sequence for exemplary | 118 | GTKDSTKDIPETPSKD |
| linker | | |
| amino acid sequence for exemplary | 119 | GRDVRQPEVKEEKPES |
| linker | | |
| amino acid sequence for exemplary | 120 | EGKSSGSGSESKSTAG |
| linker | | |
| amino acid sequence for exemplary | 121 | TPGSPAGSPTSTEEGT |
| linker | | |
| amino acid sequence for exemplary | 122 | GSEPATSGSETPGTST |
| linker | | |
| | 123- | |
| | 129 | |
| G000502 | 130 | mA*mC*mA*CAAAUACCAGUCCAGCGGUUUUAGAmGmCmUmAmGmAm |
| | | AmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUmGm |
| | | CmU*mU*mU*mU |
| G017564 | 131 | mG*mG*mC*CUGGCUGAUGAGGCCGCACAUGUUGUAGCUCCCU*mG* |
| | | mA*mA*mA*CCGUUGCUACAAUAAGGCCGUmC*mU*mG*mA*mA*mA |
| | | *mA*mGAUGUGCCGCAACGCUCUGCCG*mC*mU*mU*mC*mU*mG*C |
| | | GGCAUCGUUU*mA*mU*mC |
| G017565 | 132 | mG*mG*mC*CUGGCUGAUGAGGCCGCACAUGUUGUAGCUCCCU*mG* |
| | | mA*mA*mA*CCGUUGCUACAAUAAGGCCGUmC*mU*mG*mA*mA*mA |
| | | *mA*mGAUGUGCCGCAACGCUCUGCCmU*mU*mC*mUGGCAUCGUUU |
| | | *mA*mU*mC |
| G017566 | 133 | mG*mG*mC*CUGGCUGAUGAGGCCGCACAUGUUGUAGCUCCCU*mG* |
| | | mA*mA*mA*CCGUUGCUACAAUAAGGCCGUmC*mG*mA*mA*mA*mG |
| | | AUGUGCCGCAACGCUCUGCCmU*mU*mC*mUGGCAUCGUUU*mA*mU |
| | | *mC |
| G020055 | 134 | mG*mUGUGUCCCmUCUmCCCCACCCGUCCmGUUGmUmAmGmCUCCCm |
| | | UmGmAmAmAmCmCGUUmGmCUAmCAAUAAGmGmCCmGmUmCmGmAmA |
| | | mAmGmAmUGUGCmCGCAACGCUCUmGmCCmUmUmCmUGGCAUCGUUU |
| | | AmU*mU |
| G020361 | 135 | mG*mGC*CUGGCUmGAUmGAGGCCGCACAUmGUUGmUmAmGmCUCCC |
| | | UmCmGmAmAmAmGCCGUUmGmCUAmCAAU*A*AGmGmCCmGmUmCmG |
| | | mAmAmAmGmAmUGUGCmCGCAACGCUCUmGmCCmUmUmCmUGGCAUC |
| | | GUUU*AmU*mU |
| G020848 | 136 | mU*mGA*GGACCGmCCCmUGGGCCUGGGAGmGUUGmUmAmGmCUCCC |
| | | UmCmGmAmAmAmGCCGUUmGmCUAmCAAU*A*AGmGmCCmGmUmCmG |
| | | mAmAmAmGmAmUGUGCmCGCAACGCUCUmGmCCmUmUmCmUGGCAUC |
| | | GUUU*AmU*mU |
| G021256 | 137 | mC*mCAAGUGUCmUUCmCAGUACGAUUUGmGUUGmUmAmGmCUCCCm |
| | | UmGmAmAmAmCmCGUUmGmCUAmCAAU*AAGmGmCCmGmUmCmGmAm |
| | | AmAmGmAmUGUGCmCGCmAmAmCmGCUCUmGmCCmUmUmCmUGmGCm |
| | | AmUC*mG*mU*mU |
| G021320 | 138 | mC*mUUCACCAGmGAGmAAGCCGUCACACmGUUGmUmAmGmCUCCCm |
| | | UmGmAmAmAmCmCGUUmGmCUAmCAAU*AAGmGmCCmGmUmCmGmAm |
| | | AmAmGmAmUGUGCmCGCmAmAmCmGCUCUmGmCCmUmUmCmUGmGCm |
| | | AmUC*mG*mU*mU |
| G021536 | 139 | mC*mC*mA*mAmGUGmUmCUmUCmCAGUAmCGAUmUUGmGUUGmUmA |
| | | mGmCUCCCmUmGmAmAmAmCmCGUUmGmCUAmCAAU*AAGmGmCCmG |
| | | mUmCmGmAmAmAmGmAmUGUGCmCGmCAAmCGCUCUmGmCCmUmUmC |
| | | mUGGCAUCG*mU*mU |
| | 140 | Not used |
| G021844 | 141 | mC*mC*mA*mAmGUGmUmCUmUCmCAGUAmCGAUmUUGmGUUGmUmA |
| | | mGmCUCCCmUmUmC(L1)mGmAmCmCGUUmGmCUAmCAAU*AAGmGm |
| | | CCmGmUmC(L1)mGmAmUGUGCmCGmCAAmCGCUCUmGmCC(L1)GG |
| | | CAUCG*mU*mU |
| G021845 | 142 | mC*mU*mU*mCmACCmAmGGmAGmAAGCCmGUCAmCACmGUUGmUmA |
| | | mGmCUCCCmUmGmAmAmAmCmCGUUmGmCUAmCAAU*AAGmGmCCmG |
| | | mUmCmGmAmAmAmGmAmUGUGCmCGmCAAmCGCUCUmGmCCmUmUmC |
| | | mUGGCAUCG*mU*mU |
| G021846 | 143 | mC*mU*mU*mCmACCmAmGGmAGmAAGCCmGUCAmCACmGUUGmUmA |
| | | mGmCUCCCmUmUmC(L1)mGmAmCmCGUUmGmCUAmCAAU*AAGmGm |
| | | CCmGmUmC(L1)mGmAmUGUGCmCGmCAAmCGCUCUmGmCC(L1)GG |
| | | CAUCG*mU*mU |
| G023413 | 144 | mC*mC*mA*mAmGUGmUmCUmUCmCAGUAmCGAUmUUGmGUUGmUmA |
| | | mGmCUCCCmUmUmG(L1)mCmAmCmCGUUmGmCUAmCAAU*AAGmGm |
| | | CCmGmUmC(L1)mGmAmUGUGCmCGmCAAmCGCUCUmGmCC(L1)GG |
| | | CAUCGmU*mU |
| G023414 | 145 | mC*mC*mA*mAmGUGmUmCUmUCmCAGUAmCGAUmUUGmGUUGmUmA |
| | | mGmCUCCCmUmG(L1)mCmCmCGUUmGmCUAmCAAU*AAGmGmCCmG |
| | | mUmC(L1)mGmAmUGUGCmCGmCAAmCGCUCUmGmCC(L1)GGCAUC |
| | | GmU*mU |
| G023415 | 146 | mC*mC*mA*mAmGUGmUmCUmUCmCAGUAmCGAUmUUGmGUUGmUmA |
| | | mGmCUCCCmG(L1)mCmCGUUmGmCUAmCAAU*AAGmGmCCmGmUmC |
| | | (L1)mGmAmUGUGCmCGmCAAmCGCUCUmGmCC(L1)GGCAUCGmU* |
| | | mU |
| G023416 | 147 | mC*mC*mA*mAmGUGmUmCUmUCmCAGUAmCGAUmUUGmGUUGmUmA |
| | | mGmCUCCmG(L1)mCGUUmGmCUAmCAAU*AAGmGmCCmGmUmC(L1) |
| | | mGmAmUGUGCmCGmCAAmCGCUCUmGmCC(L1)GGCAUCGmU*mU |
| G023417 | 148 | mC*mC*mA*mAmGUGmUmCUmUCmCAGUAmCGAUmUUGmGUUGmUmA |
| | | mGmCUCC(L1)GUUmGmCUAmCAAU*AAGmGmCCmGmUmC(L1)mGm |
| | | AmUGUGCmCGmCAAmCGCUCUmGmCC(L1)GGCAUCGmU*mU |
| G023418 | 149 | mC*mC*mA*mAmGUGmUmCUmUCmCAGUAmCGAUmUUGmGUUGmUmA |
| | | mGmCUCCCmUmUmG(L1)mCmAmCmCGUUmGmCUAmCAAU*AAGmGm |
| | | CCmGmUmCmU(L1)mAmGmAmUGUGCmCGmCAAmCGCUCUmGmCC |
| | | (L1)GGCAUCGmU*mU |
| G023419 | 150 | mC*mC*mA*mAmGUGmUmCUmUCmCAGUAmCGAUmUUGmGUUGmUmA |
| | | mGmCUCCCmUmUmG(L1)mCmAmCmCGUUmGmCUAmCAAU*AAGmGm |
| | | CCmGmUmC(L1)mGmAmUGUGCmCGmCAAmCGCUCUmGmCCmU(L1) |
| | | mAGGCAUCGmU*mU |
| | 322- | NotUsed |
| | 350 | |
| G029377 | 351 | mC*mC*mA*mAmGUGmUmCUmUCmCAGUAmCGAUmUUGmGUUGmUmAmGmCU |
| | | CCCmUmGmAmAmAmCmCGUUmGmCUAmCAAUAAGmGmCCmGmUmCmGmAmAm |
| | | AmGmAmUGUGCmCGmCAAmCGCUCUmGmCCmUmUmCmUGGCAUCG*mU*mU |
| G029378 | 352 | mC*mC*mA*mAmGUGmUmCUmUCmCAGUAmCGAUmUUGmGUUGmUmAmGmCU |
| | | CCCmUmGmAmAmAmCmCGUUmGmCUAmCmAmAUAAGmGmCCmGmUmCmGmAm |
| | | AmAmGmAmUGUGCmCGmCAAmCGCUCUmGmCCmUmUmCmUGGCAUCG*mU*m |
| | | U |
| G029379 | 353 | mC*mC*mA*mAmGUGmUmCUmUCmCAGUAmCGAUmUUGmGUUGmUmAmGmCU |
| | | CCCmUmGmAmAmAmCmCGUUmGmCUAmCAmAUAAGmGmCCmGmUmCmGmAmA |
| | | mAmGmAmUGUGCmCGmCAAmCGCUCUmGmCCmUmUmCmUGGCAUCG*mU*mU |
| G029380 | 354 | mC*mC*mA*mAmGUGmUmCUmUCmCAGUAmCGAUmUUGmGUUGmUmAmGmCU |
| | | CCCmUmGmAmAmAmCmCGUUmGmCUAmCmAmAU*AAGmGmCCmGmUmCmGmA |
| | | mAmAmGmAmUGUGCmCGmCAAmCGCUCUmGmCCmUmUmCmUGGCAUCG*mU* |
| | | mU |
| G029381 | 355 | mC*mC*mA*mAmGUGmUmCUmUCmCAGUAmCGAUmUUGmGUUGmUmAmGmCU |
| | | CCCmUmGmAmAmAmCmCGUUmGmCUAmCAAdTAAGmGmCCmGmUmCmGmAmA |
| | | mAmGmAmUGUGCmCGmCAAmCGCUCUmGmCCmUmUmCmUGGCAUCG*mU*mU |
| G029382 | 356 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCmGCUCUmGmCCmUmUmCmUGGCAUCG*mU*m |
| | | U |
| G029383 | 357 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCGCUCmUmGmCCmUmUmCmUGGCAUCG*mU*m |
| | | U |
| G029384 | 358 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCGmCmUmCmUmGmCCmUmUmCmUGGCAUCG*m |
| | | U*mU |
| G029385 | 359 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCGmCmUmCUmGmCCmUmUmCmUGGCAUCG*mU |
| | | *mU |
| G029386 | 360 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCmGmCmUmCmUmGmCCmUmUmCmUGGCAUCG* |
| | | mU*mU |
| G029387 | 361 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCmAmAmCmGmCmUmCmUmGmCmCmUmUmCmUGGCAU |
| | | CG*mU*mU |
| G029388 | 362 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCGCUCUmGmCCmUmUmCmUGGCAUmCmG*mU* |
| | | mU |
| G029389 | 363 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCGCUCUmGmCCmUmUmCmUGGCmAmUmCmG*m |
| | | U*mU |
| G029390 | 364 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCmAmAmCmGmCmUmCmUmGmCmCmUmUmCmUGGCmA |
| | | mUmCmG*mU*mU |
| G029391 | 365 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCmAmAmCmGmCmUmCmUmGmCmCmUmUmCmUGGCAU |
| | | mCmG*mU*mU |
| G029392 | 366 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmUmCmGmAmA |
| | | mAmGmAmUGUGCmCGmCAAmCmGmCmUmCmUmGmCCmUmUmCmUGGCAUmCm |
| | | G*mU*mU |
| | 367- | Not Used |
| | 382 | |
| SV40 NLS | 383 | PKKKRKVE |
| SV40 NLS | 384 | KKKRKVE |
| bipartite NLS | 385 | KRTADGSEFESPKKKRKVE |
| c-myc like NLS | 386 | PAAKKKKLD |
| Nucleic acid sequence for SV40 NLS | 387 | CCCAAGAAGAAGAGGAAAGTC |
| Amino acid sequence for SV40 NLS | 388 | PKKKRKV |
| U6 promoter | 389 | TTTCCCATGATTCCTTCATATTTGCATATACGATACAAGGCTGTTAG |
| | | AGAGATAATTGGAATTAATTTGACTGTAAACACAAAGATATTAGTAC |
| | | AAAATACGTGACGTAGAAAGTAATAATTTCTTGGGTAGTTTGCAGTT |
| | | TTAAAATTATGTTTTAAAATGGACTATCATATGCTTACCGTAACTTG |
| | | AAAGTATTTCGATTTCTTGGCTTTATATATCTTGTGGAAAGGACG |
| CMV promoter | 390 | ATAGTAATCAATTACGGGGTCATTAGTTCATAGCCCATATATGGAGT |
| | | TCCGCGTTACATAACTTACGGTAAATGGCCCGCCTGGCTGACCGCCC |
| | | AACGACCCCCGCCCATTGACGTCAATAATGACGTATGTTCCCATAGT |
| | | AACGCCAATAGGGACTTTCCATTGACGTCAATGGGTGGAGTATTTAC |
| | | GGTAAACTGCCCACTTGGCAGTACATCAAGTGTATCATATGCCAAGT |
| | | CCGCCCCCTATTGACGTCAATGACGGTAAATGGCCCGCCTGGCATTA |
| | | TGCCCAGTACATGACCTTACGGGACTTTCCTACTTGGCAGTACATCT |
| | | ACGTATTAGTCATCGCTATTACCATGGTGATGCGGTTTTGGCAGTAC |
| | | ACCAATGGGCGTGGATAGCGGTTTGACTCACGGGGATTTCCAAGTCT |
| | | CCACCCCATTGACGTCAATGGGAGTTTGTTTTGGCACCAAAATCAAC |
| | | GGGACTTTCCAAAATGTCGTAACAACTGCGATCGCCCGCCCCGTTGA |
| | | CGCAAATGGGCGGTAGGCGTGTACGGTGGGAGGTCTATATAAGCAGA |
| | | GCTCGTTTAGTGAACCGTCAGATC |
| Exemplary 5′ UTR | 391 | ACATTTGCTTCTGACACAACTGTGTTCACTAGCAACCTCAAACAGAC |
| | | ACC |
| Exemplary 5′ UTR | 392 | CATAAACCCTGGCGCGCTCGCGGCCCGGCACTCTTCTGGTCCCCACA |
| | | GACTCAGAGAGAACCCACC |
| Exemplary 5′ UTR | 393 | AAGCTCAGAATAAACGCTCAACTTTGGCC |
| Exemplary 5′ UTR | 394 | CAGGGTCCTGTGGACAGCTCACCAGCT |
| Exemplary 5′ UTR | 395 | TCCCGCAGTCGGCGTCCAGCGGCTCTGCTTGTTCGTGTGTGTGTCGT |
| | | TGCAGGCCTTATTC |
| Exemplary 5′ UTR | 396 | CAGATCGCCTGGAGACGCCATCCACGCTGTTTTGACCTCCAT |
| Exemplary 5′ UTR | 397 | AGAAGACACCGGGACCGATCCAGCCTCCGCGGCCGGGAACGG |
| Exemplary 5′ UTR | 398 | TGCATTGGAACGCGGATTCCCCGTGCCAAGAGTGACTCACCG |
| Exemplary 3′ UTR | 399 | GCTCGCTTTCTTGCTGTCCAATTTCTATTAAAGGTTCCTTTGTTCCC |
| | | TAAGTCCAACTACTAAACTGGGGGATATTATGAAGGGCCTTGAGCAT |
| | | CTGGATTCTGCCTAATAAAAAACATTTATTTTCATTGC |
| Exemplary 3′ UTR | 400 | GCTGGAGCCTCGGTGGCCATGCTTCTTGCCCCTTGGGCCTCCCCCCA |
| | | GCCCCTCCTCCCCTTCCTGCACCCGTACCCCCGTGGTCTTTGAATAA |
| | | AGTCTGAGTGGGCGGC |
| Exemplary 3′ UTR | 401 | ACCAGCCTCAAGAACACCCGAATGGAGTCTCTAAGCTACATAATACC |
| | | AACTTACACTTTACAAAATGTTGTCCCCCAAAATGTAGCCATTCGTA |
| | | TCTGCTCCTAATAAAAAGAAAGTTTCTTCACATTCT |
| Exemplary 3′ UTR | 402 | TTGCCAGCCATCTGTTGTTTGCCCCTCCCCCGTGCCTTCCTTGACCC |
| | | TGGAAGGTGCCACTCCCACTGTCCTTTCCTAATAAAATGAGGAAATT |
| | | GCATCGCA |
| Exemplary 3′ UTR | 403 | GCTGCCTTCTGCGGGGCTTGCCTTCTGGCCATGCCCTTCTTCTCTCC |
| | | CTTGCACCTGTACCTCTTGGTCTTTGAATAAAGCCTGAGTAGGAAG |
| Exemplary 3′ UTR | 404 | CTGGTACTGCATGCACGCAATGCTAGCTGCCCCTTTCCCGTCCTGGG |
| | | TACCCCGAGTCTCCCCCGACCTCGGGTCCCAGGTATGCTCCCACCTC |
| | | CACCTGCCCCACTCACCACCTCTGCTAGTTCCAGACACCTCCCAAGC |
| | | ACGCAGCAATGCAGCTCAAAACGCTTAGCCTAGCCACACCCCCACGG |
| | | GAAACAGCAGTGATTAACCTTTAGCAATAAACGAAAGTTTAACTAAG |
| | | CTATACTAACCCCAGGGTTGGTCAATTTCGTGCCAGCCACACC |
| Exemplary 3′ UTR | 405 | CAAGCACGCAGCAATGCAGCTCAAAACGCTTAGCCTAGCCACACCCC |
| | | CACGGGAAACAGCAGTGATTAACCTTTAGCAATAAACGAAAGTTTAA |
| | | CTAAGCTATACTAACCCCAGGGTTGGTCAATTTCGTGCCAGCCACAC |
| | | CCTGGTACTGCATGCACGCAATGCTAGCTGCCCCTTTCCCGTCCTGG |
| | | GTACCCCGAGTCTCCCCCGACCTCGGGTCCCAGGTATGCTCCCACCT |
| | | CCACCTGCCCCACTCACCACCTCTGCTAGTTCCAGACACCTCC |
| Exemplary 3′ UTR | 406 | ACCAGCCTCAAGAACACCCGAATGGAGTCTCTAAGCTACATAATACC |
| | | AACTTACACTTTACAAAATGTTGTCCCCCAAAATGTAGCCATTCGTA |
| | | TCTGCTCCTAATAAAAAGAAAGTTTCTTCACATTCTACCAGCCTCAA |
| | | GAACACCCGAATGGAGTCTCTAAGCTACATAATACCAACTTACACTT |
| | | TACAAAATGTTGTCCCCCAAAATGTAGCCATTCGTATCTGCTCCTAA |
| | | TAAAAAGAAAGTTTCTTCACATTCT |
| Exemplary Kozak sequence | 407 | GCCRCCAUGG |
| Exemplary Kozak sequence | 408 | GCCGCCRCCAUGG |
| Exemplary poly-A sequence | 409 | AAAAAAAAAAAAAAAAAAAAAAAAAAAAAAGCGAAAAAAAAAAAAAA |
| | | AAAAAAAAAAAAAAAACCGAAAAAAAAAAAAAAAAAAAAAAAAAAAA |
| | | AAAAAAAAAAA |
| Exemplary NLS 1 | 410 | LAAKRSRTT |
| Exemplary NLS 2 | 411 | QAAKRSRTT |
| Exemplary NLS 3 | 412 | PAPAKRERTT |
| Exemplary NLS 4 | 413 | QAAKRPRTT |
| Exemplary NLS 5 | 414 | RAAKRPRTT |
| Exemplary NLS 6 | 415 | AAAKRSWSMAA |
| Exemplary NLS 7 | 416 | AAAKRVWSMAF |
| Exemplary NLS 8 | 417 | AAAKRSWSMAF |
| Exemplary NLS 9 | 418 | AAAKRKYFAA |
| Exemplary NLS 10 | 419 | RAAKRKAFAA |
| Exemplary NLS 11 | 420 | RAAKRKYFAV |
| Alternative SV40 NLS | 421 | PKKKRRV |
| Nucleoplasmin NLS | 422 | KRPAATKKAGQAKKKK |
| Exemplary coding sequence for SV40 | 423 | CCGAAGAAGAAGAGAAAGGTC |
| NLS | | |
| Exemplary coding sequence for NLS1 | 424 | CTGGCAGCAAAGAGAAGCAGAACAACA |
| Exemplary coding sequence for NLS2 | 425 | CAGGCAGCAAAGAGAAGCAGAACAACA |
| Exemplary coding sequence for NLS3 | 426 | CCGGCACCGGCAAAGAGAGAAAGAACAACA |
| Exemplary coding sequence for NLS4 | 427 | CAGGCAGCAAAGAGACCGAGAACAACA |
| Exemplary coding sequence for NLS5 | 428 | AGAGCAGCAAAGAGACCGAGAACAACA |
| Exemplary coding sequence for NLS6 | 429 | GCAGCAGCAAAGAGAAGCTGGAGCATGGCAGCA |
| Exemplary coding sequence for NLS7 | 430 | GCAGCAGCAAAGAGAGTCTGGAGCATGGCATTC |
| Exemplary coding sequence for NLS8 | 431 | GCAGCAGCAAAGAGAAGCTGGAGCATGGCATTC |
| Exemplary coding sequence for NLS9 | 432 | GCAGCAGCAAAGAGAAAGTACTTCGCAGCA |
| Exemplary coding sequence for NLS10 | 433 | AGAGCAGCAAAGAGAAAGGCATTCGCAGCA |
| Exemplary coding sequence for NLS11 | 434 | AGAGCAGCAAAGAGAAAGTACTTCGCAGTC |
| Exemplary coding sequence for | 435 | CCGAAGAAGAAGAGAAGAGTC |
| alternate SV40 NLS | | |
| G024103 | 436 | mC*mA*mA*mGmUCUmGmUCmUGmCCUAUmUCACmCGAmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCCGGCAU |
| | | CG*mU*mU |
| G024104 | 437 | mG*mG*mU*mGmAAUmAmGGmCAmGACAGmACUUmGUCmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024108 | 438 | mG*mA*mU*mUmAAAmCmCCmGGmCCACUmUUCAmGGAmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024109 | 439 | mC*mA*mG*mUmGACmAmAGmUCmUGUCUmGCCUmAUUmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024110 | 440 | mG*mU*mU*mGmAAGmGmCGmUUmUGCACmAUGCmAAAmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024111 | 441 | mU*mC*mC*mUmGUGmAmUGmUCmAAGCUmGGUCmGAGmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024112 | 442 | mC*mA*mG*mGmUUUmUmGAmAAmGUUUAmGGUUmCGUmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024113 | 443 | mA*mU*mC*mAmGAAmUmCCmUUmACUUUmGUGAmCACmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G024114 | 444 | mG*mA*mA*mGmUCCmAmUAmGAmCCUCAmUGUCmUAGmGUUG |
| | | mUmAmGmCUCCCmU(L1)mCmCGUUmGmCUAmCAAU*AAGmGmCC |
| | | mGmUmC(L1)mGmAmUGUGCmCGmCAAmCGCUCUmGmCC(L1)GGC |
| | | AUCG*mU*mU |
| G028844 | 445 | mU*mG*mU*mCmUGCmCmUAmUUmCACCGmAUUUmUGAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028845 | 446 | mU*mG*mC*mCmUAUmUmCAmCCmGAUUUmUGAUmUCUmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028846 | 447 | mC*mG*mA*mUmUUUmGmAUmUCmUCAAAmCAAAmUGUmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*U*mU |
| G028847 | 448 | mG*mU*mA*mAmGGAmUmUCmUGmAUGUGmUAUAmUCAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028848 | 449 | mA*mG*mA*mGmCAAmCmAGmUGmCUGUGmGCCUmGGAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028849 | 450 | mU*mG*mG*mAmGCAmAmCAmAAmUCUGAmCUUUmGCAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028850 | 451 | mA*mU*mG*mCmUGUmUmGUmUGmAAGGCmGUUUmGCAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028851 | 452 | mU*mU*mU*mUmGAAmAmGUmUUmAGGUUmCGUAmUCUmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028852 | 453 | mU*mU*mA*mCmUUUmGmUGmACmACAUUmUGUUmUGAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G028853 | 454 | mG*mA*mU*mUmAAAmCmCCmGGmCCACUmUUCAmGGAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G021469 | 455 | mA*mU*mA*mUmCCAmGmAAmCCmCUGACmCCUGmCCGmGUUGm |
| | | UmAmGmCUCCCmUmGmAmAmAmCmCGUUmGmCUAmCAAU*AAG |
| | | mGmCCmGmUmCmGmAmAmAmGmAmUGUGCmCGmCAAmCGCUC |
| | | UmGmCCmUmUmCmUGGCAUCG*mU*mU |
| G024739 | 456 | mA*mG*mG*mAmCCAmGmCCmUCmAGACAmCAAAmUACmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G024741 | 457 | mC*mU*mG*mCmCUCmGmGAmCGmGCAUCmUAGAmACUmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| G024743 | 458 | mA*mG*mG*mCmAGAmGmGAmGGmAGCAGmACGAmUGAmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGCU |
| | | CUmGmCCmUmUmCmUGGCAUCG*mU*mU |
| | 459- | Not Used |
| | 499 | |
| Wild-type NmeCas9 guide RNA | 500 | NNNNNNNNNNNNNNNNNNNNNNNNGUUGUAGCUCCCUUUCUCAUUUC |
| | | GGAAACGAAAUGAGAACCGUUGCUACAAUAAGGCCGUCUGAAAAGAU |
| | | GUGCCGCAACGCUCUGCCCCUUAAAGCUUCUGCUUUAAGGGGCAUCG |
| | | UUUA |
| Shortened/unmodified NmeCas9 guide | 501 | (N)₂₀₋₂₅ |
| RNA | | GUUGUAGCUCCCUGAAACCGUUGCUACAAUAAGGCCGUCGAAAGAUG |
| | | UGCCGCAACGCUCUGCCUUCUGGCAUCGUU |
| Shortened/unmodified NmeCas9 guide | 502 | (N)₂₀₋₂₅ |
| RNA | | GUUGUAGCUCCCUGAAACCGUUGCUACAAUAAGGCCGUCGAAAGAUG |
| | | UGCCGCAACGCUCUGCCUUCUGGCAUCGUUUAUU |
| Shortened/unmodified NmeCas9 guide | 503 | (N)₂₀₋₂₅ |
| RNA | | GUUGUAGCUCCCUGGAAACCCGUUGCUACAAUAAGGCCGUCGAAAGA |
| | | UGUGCCGCAACGCUCUGCCUUCUGGCAUCGUUUAUU |
| Mod-N77 conserved portion only | 504 | mGUUGmUmAmGmCUCCCmUmGmAmAmAmCmCGUUmGmCUAmCAAU*A |
| | | AGmGmCCmGmUmCmGmAmAmAmGmAmUGUGCmCGCmAmAmCmGCUCU |
| | | mGmCCmUmUmCmUGmGCmAmUC*mG*mU*mU |
| Mod-N78 conserved portion only | 505 | mGUUGmUmAmGmCUCCCmUmGmAmAmAmCmCGUUmGmCUAmCAAU*A |
| | | AGmGmCCmGmUmCmGmAmAmAmGmAmUGUGCmCGmCAAmCGCUCUmG |
| | | mCCmUmUmCmUGGCAUCG*mU*mU |
| Shortened/unmodified NmeCas9 guide | 506 | (N)₂₀₋₂₅ |
| RNA comprising linkers | | mGUUGUAGCUCCCUUC(L1)GACCGUUGCUACAAUAAGGCCGUC(L1) |
| | | GAUGUGCCGCAACGCUCUGCC(L1)GGCAUCGUU |
| Shortened/modified NmeCas9 guide | 507 | mN*mN*mN*mNmNNNmNmNNmNNmNNNNNmNNNNmNNNmGUUGmUmA |
| RNA comprising linkers | | mGmCUCCCmUmUmC(L1)mGmAmCmCGUUmGmCUAmCAAU*AAGmGm |
| | | CCmGmUmC(L1)mGmAmUGUGCmCGmCAAmCGCUCUmGmCC(L1)GG |
| | | CAUCG*mU*mU |
| Shortened/modified NmeCas9 guide | 508 | mN*mNNNNNNNNmNNNmNNNNNNNNNNNNmGUUGmUmAmGmCUCCCm |
| RNA | | UmGmAmAmAmCmCGUUmGmCUAmCAAU*AAGmGmCCmGmUmCmGmAm |
| | | AmAmGmAmUGUGCmCGCmAmAmCmGCUCUmGmCCmUmUmCmUGmGCm |
| | | AmUC*mG*mU*mU |
| G024739 unmodified sequence | 509 | AGGACCAGCCUCAGACACAAAUACGUUGUAGCUCCCUGAAACCGUUG |
| | | CUACAAUAAGGCCGUCGAAAGAUGUGCCGCAACGCUCUGCCUUCUGG |
| | | CAUCGUU |
| G024741 unmodified sequence | 510 | CUGCCUCGGACGGCAUCUAGAACUGUUGUAGCUCCCUGAAACCGUUG |
| | | CUACAAUAAGGCCGUCGAAAGAUGUGCCGCAACGCUCUGCCUUCUGG |
| | | CAUCGUU |
| G024743 unmodified sequence | 511 | AGGCAGAGGAGGAGCAGACGAUGAGUUGUAGCUCCCUGAAACCGUUG |
| | | CUACAAUAAGGCCGUCGAAAGAUGUGCCGCAACGCUCUGCCUUCUGG |
| | | CAUCGUU |
| G021536 Mod-N78 Nme Cas9 guide RNA | 512 | mN*mN*mN*mNmNNNmNmNNmNNmNNNNNmNNNNmNNNmGUUGmUmA |
| (101) | | mGmCUCCCmUmGmAmAmAmCmCGUUmGmCUAmCAAU*AAGmGmCCmG |
| | | mUmCmGmAmAmAmGmAmUGUGCmCGmCAAmCGCUCUmGmCCmUmUmC |
| | | mUGGCAUCG*mU*mU |
| G027492 Shortened Nme Cas9 guide | 513 | GUUGUAGCUCCCUUCGAAAGACCGUUGCUACAAUAAGGCCGUCGAAA |
| RNA (105) conserved portion only | | GAUGUGCCGCAACGCUCUGCCUUCUGGCAUCGUU |
| G027492 Shortened Cas9 guide RNA | 514 | (N)₂₀₋₂₅ |
| (105) | | GUUGUAGCUCCCUUCGAAAGACCGUUGCUACAAUAAGGCCGUCGAAA |
| | | GAUGUGCCGCAACGCUCUGCCUUCUGGCAUCGUU |
| G027492 Mod-N78 Nme Cas9 guide RNA | 515 | mGUUGmUmAmGmCUCCCmUmUmCmGmAmAmAmGmAmCmCGUUmGmCU |
| (105) conserved portion only | | AmCAAU*AAGmGmCCmGmUmCmGmAmAmAmGmAmUGUGCmCGmCAAm |
| | | CGCUCUmGmCCmUmUmCmUGGCAUCG*mU*mU |
| G027492 Mod-N78 Nme Cas9 guide RNA | 516 | mN*mN*mN*mNmNNNmNmNNmNNmNNNNNmNNNNmNNNmGUUGmUmA |
| (105) | | mGmCUCCCmUmUmCmGmAmAmAmGmAmCmCGUUmGmCUAmCAAU*AA |
| | | GmGmCCmGmUmCmGmAmAmAmGmAmUGUGCmCGmCAAmCGCUCUmGm |
| | | CCmUmUmCmUGGCAUCG*mU*mU |
| Exemplary sgRNA | 517 | mN*mNNNNNNNNmNNNmNNNNNNNNNNNNmGUUGmUmAmGmCUCCCm |
| | | UmGmAmAmAmCmCGUUmGmCUAmCAAUAAGmGmCCmGmUmCmGmAmA |
| | | mAmGmAmUGUGCmCGCmAmAmCmGCUCUmGmCCmUmUmCmUGmGCmA |
| | | mUC*mG*mU*mU |
| Exemplary sgRNA | 518 | mN*mNNNNNNNNmNNNmNNNNNNNNNNNNmGUUGmUmAmGmCUCCCm |
| | | UmGmAmAmAmCmCGUUmGmCUAmCAAU*AAGmGmCCmGmUmCmGmAm |
| | | AmAmGmAmUGUGCmCGCmAmAmCmGmCmUmCmUmGmCCmUmUmCmUG |
| | | mGCmAmUC*mG*mU*mU |
| Exemplary sgRNA | 519 | mN*mNNNNNNNNmNNNmNNNNNNNNNNNNmGUUGmUmAmGmCUCCCm |
| | | UmGmAmAmAmCmCGUUmGmCUAmCAAUAAGmGmCCmGmUmCmGmAmA |
| | | mAmGmAmUGUGCmCGCmAmAmCmGmCmUmCmUmGmCCmUmUmCmUGm |
| | | GCmAmUC*mG*mU*mU |
| Exemplary sgRNA | 520 | mN*mN*mN*mNmNNNmNmNNmNNmNNNNNmNNNNmNNNmGUUGmUmA |
| | | mGmCUCCCmUmUmCmGmAmAmAmGmAmCmCGUUmGmCUAmCAAUAAG |
| | | mGmCCmGmUmCmGmAmAmAmGmAmUGUGCmCGmCAAmCGCUCUmGmC |
| | | CmUmUmCmUGGCAUCG*mU*mU |
| Exemplary sgRNA | 521 | mN*mN*mN*mNmNNNmNmNNmNNmNNNNNmNNNNmNNNmGUUGmUmA |
| | | mGmCUCCCmUmUmCmGmAmAmAmGmAmCmCGUUmGmCUAmCAAU*AA |
| | | GmGmCCmGmUmCmGmAmAmAmGmAmUGUGCmCGmCAAmCGmCmUmCm |
| | | UmGmCCmUmUmCmUGGCAUCG*mU*mU |
| Exemplary sgRNA | 522 | mN*mN*mN*mNmNNNmNmNNmNNmNNNNNmNNNNmNNNmGUUGmUmA |
| | | mGmCUCCCmUmUmCmGmAmAmAmGmAmCmCGUUmGmCUAmCAAUAAG |
| | | mGmCCmGmUmCmGmAmAmAmGmAmUGUGCmCGmCAAmCGmCmUmCmU |
| | | mGmCCmUmUmCmUGGCAUCG*mU*mU |
| Exemplary sgRNA | 523 | (N)₂₀₋₂₅ |
| | | mGUUGmUmAmGmCUCCCmUmGmAmAmAmCmCGUUmGmCUAmCAAUAA |
| | | GmGmCCmGmUmCmGmAmAmAmGmAmUGUGCmCGCmAmAmCmGmCmUm |
| | | CmUmGmCCmUmUmCmUGmGCmAmUC*mG*mU*mU |
| Exemplary sgRNA | 524 | (N)2₀₋₂₅ |
| | | mGUUGmUmAmGmCUCCCmUmUmCmGmAmAmAmGmAmCmCGUUmGmCU |
| | | AmCAAUAAGmGmCCmGmUmCmGmAmAmAmGmAmUGUGCmCGmCAAmC |
| | | GmCmUmCmUmGmCCmUmUmCmUGGCAUCG*mU*mU |
| Exemplary sgRNA | 525 | mN*mN*mN*mNmNNNmNmNNmNNmNNNNNmNNNNmNNNmGUUG |
| | | mUmAmGmCUCCCmUmGmAmAmAmCmCGUUmGmCUAmCAAUAA |
| | | GmGmCCmGmUmCmGmAmAmAmGmAmUGUGCmCGmCAAmCGCU |
| | | CUmGmCCmUmUmCmUGGCAUCG*mU*mU |
| Exemplary sgRNA | 526 | mN*mN*mN*mNmNNNmNmNNmNNmNNNNNmNNNNmNNNmGUUG |
| | | mUmAmGmCUCCCmUmGmAmAmAmCmCGUUmGmCUAmCAAU*AA |
| | | GmGmCCmGmUmCmGmAmAmAmGmAmUGUGCmCGmCAAmCGmC |
| | | mUmCmUmGmCCmUmUmCmUGGCAUCG*mU*mU |
| Exemplary sgRNA | 527 | mN*mN*mN*mNmNNNmNmNNmNNmNNNNNmNNNNmNNNmGUUG |
| | | mUmAmGmCUCCCmUmGmAmAmAmCmCGUUmGmCUAmCAAUAA |
| | | GmGmCCmGmUmCmGmAmAmAmGmAmUGUGCmCGmCAAmCGmC |
| | | mUmCmUmGmCCmUmUmCmUGGCAUCG*mU*mU |
| Exemplary sgRNA (G032572; 101-mer | 528 | Mc*Mc*Ma*mAmGUGmUmCUmUCmCAGUAmCGAUmUUGmGUUGmUmA |
| as shown in FIG. 34) | | mGmCUCCCmUmGmAmAmAmCmCGUUmGmCUAmCAAUAAGmGmCCmGm |
| | | UmCmGmAmAmAmGmAmUGUGCmCGmCAAmCGmCmUmCmUmGmCCmUm |
| | | UmCmUGGCAUCG*Mu*Mu |
| Exemplary sgRNA (G031771; 105-mer | 529 | mC*mC*mA*mAmGUGmUmCUmUCmCAGUAmCGAUmUUGmGUUGmUmA |
| as shown in FIG. 35) | | mGmCUCCCmUmUmCmGmAmAmAmGmAmCmCGUUmGmCUAmCAAU |
| | | AAGmGmCCmGmUmCmGmAmAmAmGmAmUGUGCmCGmCAAmCGmCmUm |
| | | CmUmGmCCmUmUmCmUGGCAUCG*mU*mU |
| G020073 | 530 | mG*mUGUGUCCCmUCUmCCCCACCCGUCCmGUUGmUmAmGmCUCCCm |
| | | UmGmAmAmAmCmCGUUmGmCUAmCAAU*AAGmGmCCmGmUmCmGmAm |
| | | AmAmGmAmUGUGCmCGCAACGCUCUmGmCCmUmUmCmUGGCAUCGmU |
| | | *mU |
| G027492 | 531 | mC*mC*mA*mAmGUGmUmCUmUCmCAGUAmCGAUmUUGmGUUGmUmA |
| | | mGmCUCCCmUmUmCmGmAmAmAmGmAmCmCGUUmGmCUAmCAAU*AA |
| | | GmGmCCmGmUmCmGmAmAmAmGmAmUGUGCmCGmCAAmCGCUCUmGm |
| | | CCmUmUmCmUGGCAUCG*mU*mU |
| G027493 | 532 | mC*mC*mA*mAmGUGmUmCUmUCmCAGUAmCGAUmUUGmGUUGmUmAmGmCU |
| | | CCCmUmUmCmGmAmAmAmGmAmCmCGUUmGmCUAmCAAU*AAGmGmCCmGmU |
| | | mGmAmAmAmAmUGUGCmCGmCAAmCGCUCUmGmCCmUmUmCmUGGCAUCG*m |
| | | U*mU |
| G027494 | 533 | mC*mC*mA*mAmGUGmUmCUmUCmCAGUAmCGAUmUUGmGUUGmUmAmGmCU |
| | | CCCmUmUmCmGmAmAmAmGmAmCmCGUUmGmCUAmCAAU*AAGmGmCCmGmG |
| | | mAmAmAmUGUGCmCGmCAAmCGCUCUmGmCCmUmUmCmUGGCAUCG*mU*mU |
| G027495 | 534 | mC*mC*mA*mAmGUGmUmCUmUCmCAGUAmCGAUmUUGmGUUGmUmAmGmCU |
| | | CCCmUmUmCmGmAmAmAmGmAmCmCGUUmGmCUAmCAAU*AAGmGmCCmGmA |
| | | mAmAGUGCmCGmCAAmCGCUCUmGmCCmUmUmCmUGGCAUCG*mU*mU |
| G027496 | 535 | mC*mC*mA*mAmGUGmUmCUmUCmCAGUAmCGAUmUUGmGUUGmUmAmGmCU |
| | | CCCmUmGmAmAmAmCmCGUUmGmCUAmCAAU*AAGmGmCCmGmAmAmAGUGC |
| | | mCGmCAAmCGCUCUmGmCCmUmUmCmUGGCAUCG*mU*mU |

We claim:

1. A polynucleotide comprising an open reading frame (ORF), the ORF comprising:
a nucleotide sequence encoding a C-terminal N. meningitidis (Nine) Cas9 polypeptide at least 90% identical to any one of SEQ ID NOs: 29, 32-41, 224-226, 231-233, 238-240, 245-247, 252-254, 259-261, 266-268, 273-275, 280-282, 287-289, 294-296, 301-303, or 316-321, wherein the Nine Cas9 is an Nme2 Cas9, an Nme1 Cas9, or Nme3 Cas9; and
a nucleotide sequence encoding a first nuclear localization signal (NLS).

2. The polynucleotide of claim 1, wherein the ORF further comprises a nucleotide sequence encoding a second NLS.

3. The polynucleotide of claim 1, wherein the first and second NLS are independently selected from SEQ ID NO: 388 and 410-422.

4. The polynucleotide of any one of claims 1-3, wherein the polynucleotide further comprises a poly-A sequence or a polyadenylation signal sequence.

5. The polynucleotide of claim 4, wherein the poly-A sequence comprises non-adenine nucleotides.

6. The polynucleotide of any one of claims 4-5, wherein the poly-A sequence comprises 100-400 nucleotides.

7. The polynucleotide of any one of claims 4-6, wherein the poly-A sequence comprises a sequence of SEQ ID NO: 409.

8. The polynucleotide of any one of claims 1-7, wherein the ORF further comprises a nucleotide sequence encoding a linker sequence between the first NLS and the second NLS.

9. The polynucleotide of any one of claims 1-8, wherein the ORF further comprises a nucleotide sequence encoding a linker spacer sequence between the Nine Cas9 coding sequence and the NLS proximal to the Nine Cas9 coding sequence.

10. The polynucleotide of any one of claims 8-9, wherein the linker comprises at least 1, at least 2, at least 3, at least 4, at least 5, at least 6, at least 7, at least 8, at least 9, or at least 10 amino acids.

11. The polynucleotide of any one of claims 8-10, wherein the linker sequence comprises GGG or GGGS, optionally wherein the GGG or GGGS sequence is at the N-terminus of the spacer sequence.

12. The polynucleotide of any one of claims 8-11, wherein the linker sequence comprises a sequence of any one of SEQ ID NOs: 61-122.

13. The polynucleotide of any one of claims 1-12, wherein the ORF further comprises one or more additional heterologous functional domains.

14. The polynucleotide of any one of claims 1-13, wherein the Nine Cas9 has double stranded endonuclease activity.

15. The polynucleotide of any one of claims 1-14, wherein the Nine Cas9 has nickase activity.

16. The polynucleotide of any one of claims 1-14, wherein the Nine Cas9 comprises a dCas9 DNA binding domain.

17. The polynucleotide of any one of claims 1-16, wherein the NmeCas9 comprises an amino acid sequence with at least 91%, 92%, 93%, 94%, 95%, 96%, 97%, 98%, or 99% to any one of SEQ ID NOs: 1 and 4-13, 220, 227, 234, 241, 248, 255, 262, 269, 276, 283, 290, 297, or 310-315.

18. The polynucleotide of any one of claims 1-17 wherein the NmeCas9 comprises an amino acid sequence of SEQ ID NO: 1 and 4-13, 220, 227, 234, 241, 248, 255, 262, 269, 276, 283, 290, 297, or 310-315.

19. The polynucleotide of any one of claims 1-18, wherein the sequence encoding the NmeCas9 comprises a nucleotide sequence having at least 91%, 92%, 93%, 94%, 95%, 96%, 97%, 98%, or 99% identity to the nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

20. The polynucleotide of any one of claims 1-19, wherein the sequence encoding the NmeCas9 comprises a nucleotide sequence of any one of SEQ ID NOs: 15, 18-27, 29, 32-41, 221-226, 228-233, 235-240, 242-247, 249-254, 256-261, 263-268, 270-275, 277-282, 284-289, 291-296, 298-303, 304-309, or 316-321.

21. A polynucleotide comprising an open reading frame (ORF) encoding a polypeptide comprising:
a cytidine deaminase, which is optionally an APOBEC3A deaminase;
a nucleotide sequence encoding a C-terminal N. meningitidis (Nine) Cas9 nickase polypeptide at least 90% identical to any one of SEQ ID NOs: 1 and 4-13, 220, 227, 234, 241, 248, 255, 262, 269, 276, 283, 290, or 297, wherein the Nine Cas9 nickase is an Nme2 Cas9 nickase, an Nme1 Cas9 nickase, or an Nme3 Cas9 nickase; and
a nucleotide sequence encoding a first nuclear localization signal (NLS);
wherein the polypeptide does not comprise a uracil glycosylase inhibitor (UGI).

22. The polynucleotide of claim 21, wherein the ORF further comprises a nucleotide sequence encoding a second NLS.

23. The polynucleotide of any one of claims 21-22, wherein the deaminase is located N-terminal to an NLS in the polypeptide.

24. The polynucleotide of any one of claims 21-23, wherein the cytidine deaminase is located N-terminal to the first NLS and the second NLS in the polypeptide.

25. The polynucleotide of any one of claims 21-22, wherein the cytidine deaminase is located C-terminal to an NLS in the polypeptide.

26. The polynucleotide of any one of claims 23-25, wherein the cytidine deaminase is located C-terminal to the first NLS and the second NLS in the polypeptide.

27. The polynucleotide of any one of claims 21-26, wherein the ORF does not comprise a coding sequence for an NLS C-terminal to the ORF encoding the Nine Cas9.

28. The polynucleotide of any one of claims 21-26, wherein the ORF does not comprise a coding sequence C-terminal to the ORF encoding the Nine Cas9.

29. The polynucleotide of any one of claims 1-28, wherein the cytidine deaminase comprises an amino acid sequence with at least 87% identity to SEQ ID NOs: 151.

30. The polynucleotide of any one of claims 1-28, wherein the cytidine deaminase comprises an amino acid sequence with at least 80% identity to SEQ ID NOs: 152-216.

31. The polynucleotide of any one of claims 1-28, wherein the cytidine deaminase comprises an amino acid sequence with at least 80% identity to SEQ ID NO: 14.

32. The polynucleotide of any one of claims 1-31, the ORF comprises a nucleotide sequence at least 80% identical to SEQ ID NO: 42.

33. The polynucleotide of any one of claims 1-32, wherein the polynucleotide comprises a 5′ UTR with at least 90% identity to any one of SEQ ID NOs: 391-398.

34. The polynucleotide of any one of claims 1-33, wherein the polynucleotide comprises a 5′ UTR comprising any one of SEQ ID NOs: 391-398.

35. The polynucleotide of any one of claims 1-34, wherein the polynucleotide comprises a 3′ UTR with at least 90% identity to any one of SEQ ID NOs: 399-406.

36. The polynucleotide of any one of claims 1-35, wherein the polynucleotide comprises a 3′ UTR comprising any one of SEQ ID NOs: 399-306.

37. The polynucleotide of any one of claims 1-36, wherein the polynucleotide comprises a 5′ UTR and a 3′ UTR from the same source.

38. The polynucleotide of any one of claims 1-37, wherein the polynucleotide comprises a 5′ cap, optionally wherein the 5′ cap is Cap0, Cap1, or Cap2.

39. The polynucleotide of any one of claims 1-38, wherein at least 75%, 80%, 85%, 90%, 95%, 98%, 99%, or 100% of the codons of the ORF are minimal adenine codons or minimal uridine codons.

40. The polynucleotide of any one of claims 1-39, wherein the ORF comprises or consists of codons that increase translation of the mRNA in a mammal.

41. The polynucleotide of any one of claims 1-40, wherein the ORF comprises or consists of codons that increase translation of the mRNA in a human.

42. The polynucleotide of any one of claims 1-41, wherein the polynucleotide is an mRNA.

43. The polynucleotide of claim 42, wherein the ORF comprises a sequence having at least 90%, 95%, 98% or 100% identity to any one of SEQ ID NO: 29, 32-41, 224-226, 231-233, 238-240, 245-247, 252-254, 259-261, 266-268, 273-275, 280-282, 287-289, 294-296, 301-303, or 316-321.

44. The polynucleotide of any one of claims 42-43, wherein at least 10% of the uridine in the mRNA is substituted with a modified uridine.

45. The polynucleotide of any one of claims 42-43, wherein less than 10% of the uridine in the mRNA is substituted with a modified uridine.

46. The polynucleotide of claim 45, wherein the modified uridine is one or more of N1-methyl-pseudouridine, pseudouridine, 5-methoxyuridine, or 5-iodouridine.

47. The polynucleotide of claim 45, wherein the modified uridine is one or both of N1-methyl-pseudouridine or 5-methoxyuridine.

48. The polynucleotide of any one of claims 45-47, wherein the modified uridine is N1-methyl-pseudouridine.

49. The polynucleotide of any one of claims 45-47, wherein the modified uridine is 5-methoxyuridine.

50. The polynucleotide of any one of claims 44, and 46-49, wherein 15% to 45% of the uridine is substituted with the modified uridine.

51. The polynucleotide of claim 50, wherein at least 20% or at least 30% of the uridine is substituted with the modified uridine.

52. The polynucleotide of claim 51, wherein at least 80% or at least 90% of the uridine is substituted with the modified uridine.

53. The polynucleotide of claim 52, wherein 100% of the uridine is substituted with the modified uridine.

54. The polynucleotide of claim 42, wherein less than 10% of the nucleotides in the mRNA is substituted with a modified nucleotide.

55. A composition comprising the polynucleotide according to any one of claims 1-54, and at least one guide RNA (gRNA).

56. A composition comprising a first polynucleotide comprising a first open reading frame (ORF) encoding a polypeptide comprising a cytidine deaminase, optionally an APOBEC3A deaminase, and a NmeCas9 nickase, and a second polynucleotide comprising a second open reading frame encoding a uracil glycosylase inhibitor (UGI), wherein the second polynucleotide is different from the first polynucleotide, and optionally further comprising a guide RNA (gRNA).

57. The composition of claim 55 or 56, wherein the gRNA is a single guide RNA.

58. The composition of claim 55 or 56, wherein the gRNA is a dual guide RNA.

59. A composition comprising the polynucleotide according to any one of claims 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 500; and
(ii) nucleotide 36 is linked to nucleotide 65 by at least 2 nucleotides; or
(b) a shortened hairpin 1 region, wherein the shortened hairpin 1 lacks 2-10, optionally 2-8 nucleotides, wherein
(i) one or more of nucleotides 82-86 and 91-95 is deleted and optionally one or more of positions 82-96 is substituted relative to SEQ ID NO: 500; and
(ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
(i) one or more of nucleotides 113-121 and 126-134 is deleted and optionally one or more of nucleotides 113-134 is substituted relative to SEQ ID NO: 500; and
(ii) nucleotide 112 is linked to nucleotide 135 by at least 4 nucleotides;
wherein one or both nucleotides 144-145 are optionally deleted relative to SEQ ID NO: 500;
wherein at least 10 nucleotides are modified nucleotides.

60. A composition comprising the polynucleotide according to any one of claims 1-57, further comprising a single guide RNA, wherein the single guide RNA comprises a guide region and a conserved region, wherein the conserved region comprising one or more of:
(a) a shortened repeat/anti-repeat region, wherein the shortened repeat/anti-repeat region lacks 2-24 nucleotides, wherein
(i) one or more of nucleotides 37-64 is deleted and optionally substituted relative to SEQ ID NO: 500; and
(ii) nucleotide 36 is linked to nucleotide 65 by (i) a first internal linker that alone or in combination with nucleotides substitutes for 4 nucleotides, or (ii) at least 4 nucleotides; or
(b) a shortened hairpin 1 region, wherein the shortened hairpin 1 lacks 2-10, optionally 2-8 nucleotides, wherein
(i) one or more of nucleotides 82-95 is deleted and optionally substituted relative to SEQ ID NO: 500; and
(ii) nucleotide 81 is linked to nucleotide 96 by (i) a second internal linker that alone or in combination with nucleotides substitutes for 4 nucleotides, or (ii) at least 4 nucleotides; or
(c) a shortened hairpin 2 region, wherein the shortened hairpin 2 lacks 2-18, optionally 2-16 nucleotides, wherein
(i) one or more of nucleotides 113-134 is deleted and optionally substituted relative to SEQ ID NO: 500; and
(ii) nucleotide 112 is linked to nucleotide 135 by (i) a third internal linker that alone or in combination with nucleotides substitutes for 4 nucleotides, or (ii) at least 4 nucleotides;
wherein one or both nucleotides 144-145 are optionally deleted as compared to SEQ ID NO: 500;
wherein the gRNA comprises at least one of the first internal linker, the second internal linker, and the third internal linker.

61. A polypeptide encoded by the polynucleotide of any one of claims 1-60.

62. A vector comprising the polynucleotide of any one of claims 1-60.

63. An expression construct comprising a promoter operably linked to a sequence encoding the polynucleotide of any one of claims 1-60.

64. The expression construct of claim 63, wherein the promoter is an RNA polymerase promoter, optionally a bacterial RNA polymerase promoter.

65. The expression construct of claim 63 or 64, further comprising poly-A tail sequence or a polyadenylation signal sequence.

66. The expression construct of claim 65, wherein the poly-A tail sequence is an encoded poly-A tail sequence.

67. A plasmid comprising the expression construct of any one of claims 63-66.

68. A host cell comprising the vector of claim 62, the expression construct of any one of claims 63-66, or the plasmid of claim 67.

69. A pharmaceutical composition comprising the polynucleotide, composition, or polypeptide of any of claims 1-61 and a pharmaceutically acceptable carrier.

70. A kit comprising the polynucleotide, composition, or polypeptide of any of claims 1-61.

71. Use of the polynucleotide, composition, or polypeptide of any one of claims 1-61 for modifying a target gene in a cell.

72. Use of the polynucleotide, composition, or polypeptide of any one of claims 1-61 for the manufacture of a medicament for modifying a target gene in a cell.

73. The polynucleotide or composition of any one of claims 1-60, wherein the polynucleotide or composition is formulated as a lipid nucleic acid assembly composition, optionally a lipid nanoparticle.

74. A method of modifying a target gene comprising delivering to a cell the polynucleotide, polypeptide, or composition of any one of claims 1-61.

75. A method of modifying a target gene, comprising delivering to the cell one or more lipid nucleic acid assembly compositions, optionally lipid nanoparticles, comprising the polynucleotide according to any one of claims 1-60, and one or more guide RNAs.

76. The method of any one of claims 74-75, wherein at least one lipid nucleic acid assembly composition comprises lipid nanoparticle (LNPs), optionally wherein all lipid nucleic acid assembly compositions comprise LNPs.

77. The method of any one of claims 74-75, wherein at least one lipid nucleic acid assembly composition is a lipoplex composition.

78. The composition or method of any one of claims 75-77, wherein the lipid nucleic acid assembly composition comprises an ionizable lipid.

79. A method of producing a polynucleotide of any one of claims 1-54, comprising contacting the expression construct of claims 63-66 with an RNA polymerase and NTPs that comprise at least one modified nucleotide.

80. The method of claim 79, wherein NTPs comprise one modified nucleotide.

81. The method of claim 79 or 80 wherein the modified nucleotide comprises a modified uridine.

82. The method of claim 81, wherein at least 80% or at least 90% or 100% of the uridine positions are modified uridines.

83. The method of claim 81 or 82, wherein the modified uridine comprises or is a substituted uridine, pseudouridine, or a substituted pseudouridine, optionally N1-methyl-psuedouridine.

84. The method of any one of claims 79-83, wherein the expression construct comprises an encoded poly-A tail sequence.
